(19) United States
(12) Patent Application Publication
(10) Pub. No.: US 2024/0408234 A1
(43) Pub. Date: Dec. 12, 2024

(54) COMPOSITIONS AND METHODS FOR MULTIPLEX BASE EDITING IN HEMATOPOIETIC CELLS

(71) Applicant: VOR BIOPHARMA INC., Cambridge, MA (US)

(72) Inventors: Alejandra Falla, Cambridge, MA (US); Elizabeth Paik, Cambridge, MA (US); Dane Hazelbaker, Cambridge, MA (US); Tirtha Chakraborty, Cambridge, MA (US); John Lydeard, Cambridge, MA (US)

(73) Assignee: VOR BIOPHARMA INC., Cambridge, MA (US)

(21) Appl. No.: 18/691,720

(22) PCT Filed: Sep. 14, 2022
(86) PCT No.: PCT/US2022/043557

§ 371 Date: Mar. 13, 2024

Related U.S. Application Data
(60) Provisional application No. 63/244,219, filed on Sep. 14, 2021.
(60) Provisional application No. 63/278,375, filed on Nov. 11, 2021.
(60) Provisional application No. 63/343,408, filed on May 18, 2022.

(51) Int. Cl. A61K 48/00; A61K 39/00; C12N 5/0789; C12N 9/22; C12N 9/78; C12N 15/11

(52) U.S. Cl. CPC A61K 48/005; A61K 39/461; A61K 39/464429; C12N 5/0647; C12N 9/22; C12N 9/78; C12N 15/111; C12N 2310/20; C12Y 305/04002; C12Y 305/04005

(57) ABSTRACT

When a cancer patient is administered an anti-cancer therapy targeting a lineage specific cell-surface antigen (e.g., CD33 (Siglec-3), CLL-1, CD123, CD327 (Siglec-6), and/or CD312 (EMR2)), e.g., in the form of an immunotherapeutic agent, the therapy can deplete not only cancer cells expressing the lineage-specific cell-surface antigen, but also noncancerous cells expressing the lineage-specific cell-surface antigen in an “on-target, off tumor” effect. This disclosure provides, e.g., novel cells having a modification (e.g., insertion or deletion) in an endogenous lineage-specific cell-surface antigen (e.g., CD33 (Siglec-3), CLL-1, CD123, CD327 (Siglec-6), and/or CD312 (EMR2)) gene. The disclosure also provides compositions, e.g., gRNAs, that can be used to make such a modification.

### RELATED APPLICATIONS

This application claims priority to U.S. Provisional Application Ser. No. 63/343,408 filed on May 18, 2022, U.S. Provisional Application Ser. No. 63/278,375 filed on Nov. 11, 2021, and U.S. Provisional Application Ser. No. 63/244,219 filed on Sep. 14, 2021. The entire contents of each of these applications are incorporated herein by reference.

### BACKGROUND

When a cancer patient is administered an anti-cancer therapy targeting a lineage-specific cell-surface antigen (e.g., CD33 (Siglec-3), CLL-1, CD123, CD327 (Siglec-6), and/or CD312 (EMR2)), e.g., in the form of an immunotherapeutic agent, the therapy can deplete not only cancer cells expressing the lineage-specific cell-surface antigen, but also noncancerous cells expressing the lineage-specific cell-surface antigen in an “on-target, off-tumor” effect. Since certain noncancerous hematopoietic cells typically express CD33 (Siglec-3), CLL-1, CD123, CD327 (Siglec-6), and/or CD312 (EMR2), they can be targeted by such anti-cancer therapeutics, and the loss of the noncancerous CD33 (Siglec-3)+, CLL-1+, CD123+, CD327 (Siglec-6)+, and/or CD312 (EMR2)+cells can deplete and impair the hematopoietic system of the patient. To address this depletion, the subject can be administered rescue cells (e.g., hematopoietic stem cells (HSCs) and/or hematopoietic progenitor cells (HPCs)) comprising a modification in the CD33 (Siglec-3), CLL-1, CD123, CD327 (Siglec-6), and/or CD312 (EMR2) gene, e.g., a genetic edit that results in the rescue cells having reduced or eliminated expression of the respective gene, or a modification of an epitope of the protein encoded by the respective gene that diminishes the binding of the therapeutic agent to the protein. These CD33 (Siglec-3), CLL-1, CD123, CD327 (Siglec-6), and/or CD312 (EMR2)-modified cells can thus be resistant to the anti-cancer therapy, and can therefore repopulate the hematopoietic system during or after anti-cancer therapy. HSCs and HPCs can be modified using various gene editing technologies, including, for example, CRISPR/Cas technologies. However, conventional CRISPR/Cas technologies are associated with certain limitations, for example, off-target editing effects (OTEs), chromosomal rearrangements, and genotoxicity due to simultaneous double-strand break (DSB) induction at multiple loci. The present disclosure addresses the need for safe and effective methods to achieve gene editing, including multiplex editing, of cells for therapeutic applications.

### SUMMARY

Provided herein are treatment modalities involving multiplex modification of the endogenous CD33 (Siglec-3), CLL-1, CD123, CD327 (Siglec-6), and/or CD312 (EMR2) gene(s), and strategies, compositions, and methods for making and using the same. Aspects of this disclosure are directed to the modification of DNA, such as multiplex modification of DNA, in a cell using one or more guide RNAs (gRNAs) to direct a base editor, e.g., a nuclease-impaired or partially nuclease impaired enzyme (e.g., RNA-guided CRISPR/Cas protein) fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide, to a target location on the DNA wherein the base editor provides an editing event.

Particular aspects of this disclosure provide methods of multiplex base editing, e.g., methods of using certain gRNAs and/or gene editing enzymes (e.g., RNA-guided CRISPR/Cas protein, Base editors, etc.) provided herein to create genetically engineered cells, e.g., cells having one, two, or multiple modifications in a gene encoding a cell-surface antigen. In some embodiments, the methods of multiplex base editing provided herein can be used to create genetically engineered cells having modifications in multiple genes encoding cell-surface antigens. Accordingly, use of the methods provided herein can enable the efficient removal of one, two, or multiple cell-surface antigens from cells for therapeutic applications, such as immunotherapy.

Without wishing to be bound by theory, removal of cell-surface antigens by multiplex base editing the genome of hematopoietic stem and progenitor cells (HSPCs) in allogeneic transplants is a new and advantageous approach to enable post-transplant targeted therapies in diseases, such as acute myeloid leukemia (AML). In some embodiments, this disclosure provides methods that allow for compatible therapeutic modalities to specifically target leukemic cells while protecting the target antigen null allogenic draft. However, given that one of the known hurdles in treating AML is tumor antigen heterogeneity, modifying, e.g., removing, one surface target may not be sufficient to achieve efficacy in AML and avoid potential antigen escape. In such instances, combinatorial therapies targeting multiple antigens, e.g., multiple cell-surface antigens, may provide greater efficacy in AML treatment and help avoid potential antigen escape. Accordingly, aspects of this disclosure provide a multiplex base editing approach using cytosine base editors (CBE) to simultaneously induce gene knock-out (KO) of clinically relevant AML surface antigens in CD34+ HSPCs from healthy donors. Such methods can enable administration of combinatorial targeted therapeutics with reduced on-target, off-tumor toxicity for AML patients. Moreover, the simultaneous delivery of base editing guides according to the methods provided herein can preserve the health, expansion, and stemness of HSPCs, which can facilitate the process and manufacturing of combinatorial targeted cells for therapeutic applications. In some embodiments, the methods provided herein can achieve high base editing efficiency, robust surface protein knockout (KO), and no detection of balanced translocation of multiplex edited cells. In some embodiments, this disclosure provides methods for multiplex base editing in CD34+HSPCs of one, two, or multiple cell surface targets (e.g., cell-surface antigens), thereby offering a valuable, safe, and efficacious alternative to engineer the next generation of cell transplants to treat AML patients. Accordingly, aspects of this disclosure provide methods for multiplex base editing in human hematopoietic stem and progenitor cells (HSPCs) which enable the efficient removal of one, two, or multiple cell-surface antigens in acute myeloid leukemia (AML) immunotherapy.

In some embodiments, this disclosure provide methods of multiplex base editing to modify, e.g., remove, one, two, or multiple cell-surface antigens. Exemplary cell-surface antigens include, but are not limited to, CD1a, CD1b, CD1c, CD1d, CD1e, CD2, CD3d, CD3e, CD3g, CD4, CD5, CD6, CD7, CD8a, CD8b, CD9, CD10, CD11a, CD11b, CD11c, CD11d, CD13, CD14, CD15, CD16a, CD16b, CD17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c,CD61, CD62E, CD62L, CD62P, CD63, CD64, CD65s, CD66a, CD66b, CD66c, CD66d, CD66e, CD66f, CD67, CD68, CD69, CD70, CD71, CD72, CD73, CD74, CD75, CD75s,CD77, CD79a, CD79b, CD80, CD81, CD82, CD83, CD84, CD85a, CD85b, CD85c, CD85d, CD85e, CD85f, CD85g, CD85h, CD85i, CD85j, CD85k, CD86, CD87, CD88, CD89, CD90, CD91, CD92, CD93, CD94, CD95, CD96, CD97, CD98, CD99, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD122, CD123, CD124, CD125, CD126, CD127, CD128a, CD128b, CD129, CD130, CD131, CD132, CD133,CD134, CD135, CD136, CD137, CD138, CD139,CD140a, CD140b, CD141, CD142, CD143, CD144, CD146, CD147, CD148, CD150, CD151, CD152, CD153, CD154, CD155, CD156a, CD156b, CD156c, CD157, CD158a, CD158b1, CD158b2, CD158c,CD158d, CD158e1, CD158e2, CD158f, CD158g,CD158h, CD158i, CD158j, CD158k, CD159a, CD159c, CD160, CD161, CD162, CD163, CD164, CD165,CD166, CD167a, CD167b, CD168, CD169, CD170, CD171, CD172a, CD172b, CD172g, CD173, CD174, CD175,CD175s, CD176, CD177, CD178, CD179a, CD179b, CD180, CD181, CD182, CD183, CD184, CD185, CD186, CD191, CD192, CD193, CD194, CD195, CD196, CD197, CD198, CD199, CD200, CD201, CD202b, CD203c, CD204, CD205, CD206, CD207, CD208, CD209, CD210a, CD210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD238, CD239, CD240CE, CD240D, CD241, CD242, CD243, CD244, CD245,CD246, CD247, CD248, CD249, CD252, CD253, CD254, CD256, CD257, CD258, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300d, CD300e, CD300f, CD300g, CD301, CD302, CD303,CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD360, CD361, CD362, CD363, CD364, CD365, CD366, CD367, CD368, CD369, CD370, and CD371, or any combination thereof.

In some embodiments, this disclosure provides methods of multiplex base editing to modify, e.g., one, two, three, four, or more cell-surface antigens selected from the group consisting of CD33 (Siglec-3), CLL-1, CD123, CD327 (Siglec-6), CD312 (EMR2), and any combination thereof.

In some embodiments, the cell-surface antigen is CD33 (Siglec-3).

In some embodiments, the cell-surface antigen is CLL-1.

In some embodiments, the cell-surface antigen is CD123.

In some embodiments, the cell-surface antigen is CD327 (Siglec-6).

In some embodiments, the cell-surface antigen is CD312 (EMR2).

Particular aspects of this disclosure provide methods of multiplex base editing, e.g., methods of using certain gRNAs and/or gene editing enzymes provided herein to create genetically engineered cells, e.g., cells having multiple modifications in the endogenous CD33 (Siglec-3), CLL-1, CD123, CD327 (Siglec-6), and/or CD312 (EMR2) gene(s).

Without wishing to be bound by theory, CRISPR/Cas technologies can be associated with various limitations including, for example, off-target editing effects (OTEs), chromosomal rearrangements, and genotoxicity due to simultaneous double-strand break (DSB) induction at multiple loci. OTEs may include unintended point mutations, deletions, insertions, inversions, and translocations at or near the target sequence. Chromosomal translocations can arise when DNA ends from double-strand breaks (DSBs) on two heterologous chromosomes are improperly joined.

The methods of multiplex base editing provided herein have advantages over conventional CRISPR/Cas technologies, at least because base editing substantially reduces the frequency of DSB formation as compared to conventional CRISPR/Cas technologies such that the methods described herein can be used without significant risk of translocations.

The methods of multiplex base editing provided herein can be used to produce genetically engineered cells having a lower overall translocation rate as compared to the use of a conventional CRISPR/Cas technology. Such cells can have 0% translocations, e.g., as assessed by a translocation analysis assay, such as a RhampSeq assay. In certain embodiments, cells produced by a multiplex base editing method provided herein can have 0% translocations, or an undetectable level of translocations, and an on-target editing efficiency of at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% or more, e.g., for modification of the endogenous CD33 (Siglec-3), CLL-1, CD123, CD327 (Siglec-6), and/or CD312 (EMR2) gene(s).

In certain embodiments, the on-target editing efficiency is at least about 80%, at least about 85%, at least about 90%, at least about 95%, or at least about 99% or more, e.g., for modification of the endogenous CD33 (Siglec-3), CLL-1, CD123, CD327 (Siglec-6), and/or CD312 (EMR2) gene(s). In certain embodiments, the on-target editing efficiency is for knockout of the endogenous CD33 (Siglec-3), CLL-1, CD123, CD327 (Siglec-6), and/or CD312 (EMR2) gene(s).

In certain embodiments, multiplex base editing may comprise comboplexing by utilizing a base editor and a CRISPR nuclease without any risk of translocations, for example, a CRISPR nuclease including a Cas9 or a Cas12a nuclease. In certain embodiments, multiplex base editing may comprise simultaneous delivery of a Cytosine Base Editor (CBE) and a Cpf1 nuclease. In certain embodiments, multiplex base editing may comprise simultaneous delivery of a Cytosine Base Editor (CBE) and/or an Adenine Base Editor (ABE). In certain embodiments, multiplex base editing has no significant impact on cell viability and/or cell expansion.

In some embodiments, multiplex modification of the endogenous CD33 (Siglec-3), CLL-1, CD123, CD327 (Siglec-6), and/or CD312 (EMR2) gene(s) occurs simultaneously.

In particular, provided herein are methods for multiplex base editing, comprising: (i) providing a cell, and (ii) introducing into the cell (a) one or more guide RNAs (gRNAs) that target CD33 (Siglec-3), one or more gRNAs that target CLL-1, one or more gRNAs that target CD123, one or more gRNAs that target CD327 (Siglec-6), and/or one or more gRNAs that target CD312 (EMR2); and (b) a base editor that binds the one or more gRNAs, wherein the one or more gRNAs are configured to provide an editing event within the same or different target domains, thereby producing a genetically engineered cell.

In particular, provided herein are methods for producing a genetically engineered cell, comprising: (i) providing a cell, and (ii) introducing into the cell (a) one or more guide RNAs (gRNAs) that target CD33 (Siglec-3), one or more gRNAs that target CLL-1, one or more gRNAs that target CD123, one or more gRNAs that target CD327 (Siglec-6), and/or one or more gRNAs that target CD312 (EMR2); and (b) a base editor that binds the one or more gRNAs, wherein the one or more gRNAs are configured to provide an editing event within the same or different target domains, thereby producing a genetically engineered cell.

In particular, provided herein are methods for multiplex base editing, comprising: (i) providing a cell, and (ii) introducing into the cell (a) one or more guide RNAs (gRNAs) that target CD33 (Siglec-3), one or more gRNAs that CLL-1, one or more gRNAs that CD123, one or more gRNAs that CD327 (Siglec-6), and/or one or more gRNAs that CD312 (EMR2); and (b) a base editor that binds the one or more gRNAs, wherein the one or more gRNAs are configured to provide an editing event within different target domains, thereby producing a genetically engineered cell.

In particular, provided herein are methods for multiplex base editing, comprising: (i) providing a cell, and (ii) introducing into the cell (a) one or more guide RNAs (gRNAs) that target CD33; (b) one or more gRNAs that target CLL-1 and/or one or more gRNAs that target CD123; and (c) a base editor that binds the one or more gRNAs, wherein the one or more gRNAs are configured to provide an editing event within different target domains, thereby producing a genetically engineered cell.

In particular, provided herein are methods for producing a genetically engineered cell, comprising: (i) providing a cell, and (ii) introducing into the cell (a) one or more guide RNAs (gRNAs) that target CD33; (b) one or more gRNAs that target CLL-1 and/or one or more gRNAs that target CD123; and (c) a base editor that binds the one or more gRNAs, wherein the one or more gRNAs are configured to provide an editing event within different target domains, thereby producing a genetically engineered cell.

In particular, provided herein are methods for multiplex base editing, comprising: (i) providing a cell, and (ii) introducing into the cell (a) one or more guide RNAs (gRNAs) that target CD33 (Siglec-3); (b) one or more gRNAs that target CLL-1, one or more gRNAs that target CD123, one or more gRNAs that target CD327 (Siglec-6), and/or one or more gRNAs that target CD312 (EMR2); and (c) a base editor that binds the one or more gRNAs, wherein the one or more gRNAs are configured to provide an editing event within different target domains, thereby producing a genetically engineered cell.

In particular, provided herein are methods for producing a genetically engineered cell, comprising: (i) providing a cell, and (ii) introducing into the cell (a) one or more guide RNAs (gRNAs) that target CD33 (Siglec-3); (b) one or more gRNAs that target CLL-1, one or more gRNAs that target CD123, one or more gRNAs that target CD327 (Siglec-6), and/or one or more gRNAs that target CD312 (EMR2); and (c) a base editor that binds the one or more gRNAs, wherein the one or more gRNAs are configured to provide an editing event within the same or different target domains, thereby producing a genetically engineered cell.

In particular, provided herein are methods for triplex base editing, comprising: (i) providing a cell, and (ii) introducing into the cell (a) a plurality of gRNAs configured to provide simultaneous editing events within at least three different genomic targets; and (d) a base editor that binds the plurality of gRNAs, thereby producing a genetically engineered cell.

In particular, provided herein are methods for triplex base editing, comprising: (i) providing a cell, and (ii) introducing into the cell (a) one or more gRNAs that target CD33 (Siglec-3); (b) one or more gRNAs that target CLL1; (c) one or more gRNAs that target CD123; and (d) a base editor that binds the one or more gRNAs, wherein the one or more gRNAs are configured to provide simultaneous editing events within at least three different target domains, thereby producing a genetically engineered cell.

In particular, provided herein are methods for producing a genetically engineered cell, comprising: (i) providing a cell, and (ii) introducing into the cell (a) a plurality of gRNAs configured to provide simultaneous editing events within at least three different genomic targets; and (d) a base editor that binds the plurality of gRNAs, thereby producing a genetically engineered cell.

In particular, provided herein are methods for producing a genetically engineered cell, comprising: (i) providing a cell, and (ii) introducing into the cell (a) one or more gRNAs that target CD33 (Siglec-3); (b) one or more gRNAs that target CLL1; (c) one or more gRNAs that target CD123; and (d) a base editor that binds the one or more gRNAs, wherein the one or more gRNAs are configured to provide simultaneous editing events within at least three different target domains, thereby producing a genetically engineered cell.

In particular, provided herein are methods for quadruplex base editing, comprising: (i) providing a cell, and (ii) introducing into the cell (a) a plurality of gRNAs configured to provide simultaneous editing events within at least four different genomic targets; and (d) a base editor that binds the plurality of gRNAs, thereby producing a genetically engineered cell.

In particular, provided herein are methods for quadruplex base editing, comprising: (i) providing a cell, and (ii) introducing into the cell (a) one or more gRNAs that target CD33 (Siglec-3); (b) one or more gRNAs that target CLL1; (c) one or more gRNAs that target CD123; (d) one or more gRNAs that target CD312 (EMR2); (e) a base editor that binds the one or more gRNAs, wherein the one or more gRNAs are configured to provide simultaneous editing events within at least four different target domains, thereby producing a genetically engineered cell.

In particular, provided herein are methods for producing a genetically engineered cell, comprising: (i) providing a cell, and (ii) introducing into the cell (a) a plurality of gRNAs configured to provide simultaneous editing events within at least four different genomic targets; and (d) a base editor that binds the plurality of gRNAs, thereby producing a genetically engineered cell.

In particular, provided herein are methods for producing a genetically engineered cell, comprising: (i) providing a cell, and (ii) introducing into the cell (a) one or more gRNAs that target CD33 (Siglec-3); (b) one or more gRNAs that target CLL1; (c) one or more gRNAs that target CD123; (d) one or more gRNAs that target CD312 (EMR2);(e) a base editor that binds the one or more gRNAs, wherein the one or more gRNAs are configured to provide simultaneous editing events within at least four different target domains, thereby producing a genetically engineered cell.

In some embodiments, multiplex modification of DNA comprises cycling or repeating steps of DNA modification on a cell to create a cell having multiple modifications of DNA within the cell. In other embodiments, multiplex modification of DNA does not comprise cycling or repeating steps of DNA modification on a cell to create a cell having multiple modifications of DNA within the cell.

Some aspects of this disclosure provide, e.g., novel cells, e.g., HSCs or HPCs, having a modification (e.g., a plurality of modifications) in the endogenous CD33 (Siglec-3), CLL-1, CD123, CD327 (Siglec-6), and/or CD312 (EMR2) gene(s). Some aspects of this disclosure provide, e.g., novel cells, e.g., HSCs or HPCs, having a modification (e.g., substitution, insertion, or deletion) in the endogenous CD33 (Siglec-3), CLL-1, CD123, CD327 (Siglec-6), and/or CD312 (EMR2) gene(s). Some aspects of this disclosure provide, e.g., novel cells, e.g., HSCs or HPCs, having a modification (e.g., a stop codon or a mutated splice site) in the endogenous CD33 (Siglec-3), CLL-1, CD123, CD327 (Siglec-6), and/or CD312 (EMR2) gene(s).

Some aspects of this disclosure provide, cell populations comprising a plurality of genetically engineered hematopoietic stem or progenitor cells, for example, wherein at least a portion of the cells comprise: (i) an edited CD33 gene and an edited CLL-1 gene; (ii) an edited CD33 gene and an edited CD123 gene; or (iii) an edited CD33 gene, an edited CLL-1 gene, and an edited CD123 gene.

Some aspects of this disclosure provide, cell populations comprising a plurality of genetically engineered hematopoietic stem or progenitor cells, for example, wherein at least a portion of the cells comprise: (i) an edited CD33 (Siglec-3) gene; (ii) an edited CLL-1 gene; (iii) an edited CD123 gene; (iv) an edited CD327 (Siglec-6) gene; (v) an edited CD312 (EMR2) gene; (vi) an edited CD33 (Siglec-3) gene and an edited CLL-1 gene; (vii) an edited CD33 (Siglec-3) gene and an edited CD123 gene; (viii) an edited CD33 (Siglec-3) gene and an edited CD327 (Siglec-6) gene; (ix) an edited CD33 (Siglec-3) gene and an edited CD312 (EMR2) gene; (x) an edited CD33 (Siglec-3) gene, an edited CLL-1 gene, and an edited CD123 gene; (xi) an edited CD33 (Siglec-3) gene, an edited CLL-1 gene, an edited CD123 gene, and an edited CD327 (Siglec-6) gene; (xii) an edited CD33 (Siglec-3) gene, an edited CLL-1 gene, an edited CD123 gene, an edited CD327 (Siglec-6) gene, and an edited CD312 (EMR2) gene; or (xiii) an edited CD33 (Siglec-3) gene, an edited CLL-1 gene, an edited CD123 gene, an edited CD327 (Siglec-6) gene, and/or an edited CD312 (EMR2) gene.

Such novel cells can have 0% translocations, e.g., as assessed by a translocation analysis assay, such as a RhampSeq assay. In certain embodiments, novel cells (e.g., HSCs or HPCs) produced by a multiplex base editing method provided herein can have 0% translocations, or an undetectable level of translocations, and an on-target editing efficiency of at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% or more, e.g., for a modification (e.g., a plurality of modifications) in the endogenous CD33 (Siglec-3), CLL-1, CD123, CD327 (Siglec-6), and/or CD312 (EMR2) gene(s). In certain embodiments, the on-target editing efficiency is at least about 80%, at least about 85%, at least about 90%, at least about 95%, or at least about 99% or more, e.g., for a modification (e.g., a plurality of modifications) in the endogenous CD33 (Siglec-3), CLL-1, CD123, CD327 (Siglec-6), and/or CD312 (EMR2) gene(s).

Some aspects of this disclosure also provide compositions, e.g., gene editing enzymes, gRNAs, and combinations thereof, that can be used to make such a modification. Some aspects of this disclosure provide methods of using the compositions provided herein, e.g., methods of using certain gRNAs provided to create genetically engineered cells, e.g., cells having a modification in the endogenous CD33 (Siglec-3), CLL-1, CD123, CD327 (Siglec-6), and/or CD312 (EMR2) gene(s).

Some aspects of this disclosure provide genetically engineered cells having a modification in an endogenous cell-surface antigen gene. In some embodiments, the genetically engineered cells have one, two, or multiple modifications in a cell-surface antigen gene, but are not limited to, CD1a, CD1b, CD1c, CD1d, CD1e, CD2, CD3d, CD3e, CD3g, CD4, CD5, CD6, CD7, CD8a, CD8b, CD9, CD10, CD11a, CD11b, CD11c, CD11d, CD13, CD14, CD15, CD16a, CD16b, CD17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c,CD61, CD62E, CD62L, CD62P, CD63, CD64, CD65s, CD66a, CD66b, CD66c, CD66d, CD66e, CD66f, CD67, CD68, CD69, CD70, CD71, CD72, CD73, CD74, CD75, CD75s,CD77, CD79a, CD79b, CD80, CD81, CD82, CD83, CD84, CD85a, CD85b, CD85c, CD85d, CD85e, CD85f, CD85g, CD85h, CD85i, CD85j, CD85k, CD86, CD87, CD88, CD89, CD90, CD91, CD92, CD93, CD94, CD95, CD96, CD97, CD98, CD99, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD122, CD123, CD124, CD125, CD126, CD127, CD128a, CD128b, CD129, CD130, CD131, CD132, CD133,CD134, CD135, CD136, CD137, CD138, CD139,CD140a, CD140b, CD141, CD142, CD143, CD144, CD146, CD147, CD148, CD150, CD151, CD152, CD153, CD154, CD155, CD156a, CD156b, CD156c, CD157, CD158a, CD158b1, CD158b2, CD158c,CD158d, CD158e1, CD158e2, CD158f, CD158g,CD158h, CD158i, CD158j, CD158k, CD159a, CD159c, CD160, CD161, CD162, CD163, CD164, CD165,CD166, CD167a, CD167b, CD168, CD169, CD170, CD171, CD172a, CD172b, CD172g, CD173, CD174, CD175,CD175s, CD176, CD177, CD178, CD179a, CD179b, CD180, CD181, CD182, CD183, CD184, CD185, CD186, CD191, CD192, CD193, CD194, CD195, CD196, CD197, CD198, CD199, CD200, CD201, CD202b, CD203c, CD204, CD205, CD206, CD207, CD208, CD209, CD210a, CD210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD238, CD239, CD240CE, CD240D, CD241, CD242, CD243, CD244, CD245,CD246, CD247, CD248, CD249, CD252, CD253, CD254, CD256, CD257, CD258, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300d, CD300e, CD300f, CD300g, CD301, CD302, CD303,CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD360, CD361, CD362, CD363, CD364, CD365, CD366, CD367, CD368, CD369, CD370, CD371, or any combination thereof. In some embodiments, the genetically engineered cells have reduced expression levels of one or more gene products of a cell-surface antigen (e.g., an mRNA, a protein, or a combination thereof). In some embodiments, the genetically engineered cells lack one or more gene products of a cell-surface antigen (e.g., an mRNA, a protein, or a combination thereof). In some embodiments, the detection of one or more gene modifications and/or a decrease in expression levels as described herein may be based on one or more measurements or assays, for example, a quantitative or semi-quantitative value of expression of a single gene, for example, reflective of the signal obtained from a quantitative or semi-quantitative assay detecting the abundance of a gene product (e.g., a protein or a nucleic acid transcript encoded by the gene). Suitable assays for the detection of gene expression products are well known to those of skill in the art and include, for example, western blots, ELISA, RT-PCR (e.g., end-point RT-PCR, real-time PCR, or qPCR), protein or nucleic acid microarray, and massive parallel sequencing assays. However, any suitable assay may be used based on hybridization, specific binding (e.g., antibody binding), or any other technique. Particular aspects of this disclosure provide methods of using the compositions provided herein, e.g., methods of using certain gRNAs and/or gene editing enzymes provided to create genetically engineered cells, e.g., cells having multiple modifications in the endogenous CD33 (Siglec-3), CLL-1, CD123, CD327 (Siglec-6), and/or CD312 (EMR2) gene(s).

Some aspects of this disclosure provide methods of administering genetically engineered cells provided herein, e.g., cells having a modification in the endogenous CD33 (Siglec-3), CLL-1, CD123, CD327 (Siglec-6), and/or CD312 (EMR2) gene(s), to a subject in need thereof. In some embodiments, the subject has, or has been diagnosed with, a cancer or a premalignant condition. In some embodiments, the cancer is a hematologic malignancy. In some embodiments, the pre-malignant condition is myelodysplastic syndrome. In some embodiments, the cancer or the pre-malignant condition is characterized by expression of CD33 (Siglec-3), CLL-1, CD123, CD327 (Siglec-6), and/or CD312 (EMR2) on the surface of malignant cells in the subject.

Some aspects of this disclosure provide strategies, compositions, methods, and treatment modalities for the treatment of patients having cancer and receiving or in need of receiving an anti-cancer therapy, such as an anti-CD33 (Siglec-3), anti-CLL-1, anti-CD123, anti-CD327 (Siglec-6), and/or anti-CD312 (EMR2) therapy. In some embodiments, the subject has, or has been diagnosed with, a cancer or a premalignant condition. In some embodiments, the cancer is a hematologic malignancy. In some embodiments, the pre-malignant condition is myelodysplastic syndrome. In some embodiments, the cancer or the pre-malignant condition is characterized by expression of CD33 (Siglec-3), CLL-1, CD123, CD327 (Siglec-6), and/or CD312 (EMR2) on the surface of malignant cells in the subject.

### Enumerated Embodiments

1. A gRNA comprising a targeting domain which binds a target domain of Tables 1-19.
2. A gRNA comprising a targeting domain which binds a target domain comprising a nucleic acid sequence of any one of SEQ ID NOs: 1-2021.
3. A gRNA comprising a targeting domain capable of directing editing of a target domain of Tables 1-19.
4. A gRNA comprising a targeting domain, wherein the targeting domain comprises a nucleic acid sequence of any one of SEQ ID NOs: 1-2021.
5. The gRNA of any one of the preceding embodiments, which binds a target domain in a CD33 (Siglec-3) gene.
6. The gRNA of any one of the preceding embodiments, which binds a target domain in a CLL-1 gene.
7. The gRNA of any one of the preceding embodiments, which binds a target domain in a CD123 gene.
8. The gRNA of any one of the preceding embodiments, which binds a target domain in a CD327 (Siglec-6) gene.
9. The gRNA of any one of the preceding embodiments, which binds a target domain in a CD312 (EMR2) gene.
10. The gRNA of any one of the preceding embodiments, which binds a target domain in a CD327 (Siglec-6) gene.
11. The gRNA of any one of the preceding embodiments, wherein the targeting domain is configured to provide an editing event within the target domain under conditions suitable for the gRNA to form a complex with a gene editing enzyme, thus forming a gRNA:enzyme complex, and for the gRNA:enzyme complex to bind the target domain in a target nucleic acid molecule.
12. The gRNA of embodiment 11, wherein the gene editing enzyme comprises an endonuclease.
13. The gRNA of embodiment 12, wherein the endonuclease comprises a Cas endonuclease.
14. The gRNA of embodiment 12 or 13, wherein the endonuclease comprises a catalytically inactive Cas molecule.
15. The gRNA of any one of embodiments 12-14, wherein the endonuclease comprises a dead Cas (dCas).
16. The gRNA of embodiment 15, wherein the endonuclease comprises a dead Cas9 (dCas9).
17. The gRNA of any one of embodiments 12-14, wherein the endonuclease comprises a nickase (nCas).
18. The gRNA of embodiment 17, wherein the endonuclease comprises an nCas9.
19. The gRNA of any one of embodiments 12-18, wherein the endonuclease comprises a dCas or an nCas fused to one or more uracil glycosylase inhibitor (UGI) domains.
20. The gRNA of any one of embodiments 12-19, wherein the endonuclease comprises a dCas or an nCas fused to a base editor (BE).
21. The gRNA of any one of embodiments 12-20, wherein the endonuclease comprises a dCas or an nCas fused to an adenine base editor (ABE).
22. The gRNA of embodiment 21, wherein the ABE comprises an adenine deaminase enzyme.
23. The gRNA of any one of embodiments 12-20, wherein the endonuclease comprises a dCas or an nCas fused to a cytosine base editor (CBE).
24. The gRNA of embodiment 23, wherein the CBE comprises a cytidine deaminase enzyme.
25. The gRNA of any one of embodiments 11-24, wherein the nucleic acid molecule is comprised in the genomic DNA of a cell.
26. The gRNA of embodiment 25, wherein the cell is a mammalian cell.
27. The gRNA of embodiment 25 or 26, wherein the cell is a human cell.
28. The gRNA of embodiment 25 or 26, wherein the cell is a CD34+ cell.
29. The gRNA of embodiment 25 or 26, wherein the cell is a hematopoietic cell.
30. The gRNA of embodiment 25 or 26, wherein the cell is a hematopoietic stem cell.
31. The gRNA of embodiment 25 or 26, wherein the cell is a hematopoietic progenitor cell.
32. The gRNA of embodiment 25 or 26, wherein the cell is an immune effector cell.
33. The gRNA of embodiment 25 or 26, wherein the cell is a lymphocyte.
34. The gRNA of embodiment 25 or 26, wherein the cell is a T-lymphocyte.
35. The gRNA of embodiment 25 or 26, wherein the cell is a natural killer (NK) cell.
36. The gRNA of embodiment 25 or 26, wherein the cell is a stem cell.
37. The gRNA of embodiment 36, wherein, the stem cell is an embryonic stem cell (ESC), an induced pluripotent stem cell (iPSC), a mesenchymal stem cell, or a tissue-specific stem cell.
38. The gRNA of any one of embodiments 11-37, wherein the editing event comprises a chemical alteration to a nucleobase.
39. The gRNA of embodiment 38, wherein the editing event comprises the deamination of a cytosine.
40. The gRNA of embodiment 38, wherein the editing event comprises the deamination of an adenine.
41. The gRNA of embodiment 38, wherein the editing event comprises a nucleobase transition.
42. The gRNA of embodiment 38, wherein the editing event comprises a nucleobase transversion.
43. The gRNA of embodiment 38, wherein the editing event comprises converting a cytosine-guanine (C-G) base pair into a thymine-adenine (T-A) base pair within the target nucleic acid molecule.
44. The gRNA of embodiment 38, wherein the editing event comprises converting a thymine-adenine (T-A) base pair into a cytosine-guanine (C-G) base pair within the target nucleic acid molecule.
45. The gRNA of embodiment 38, wherein the editing event comprises introducing a premature STOP codon within the target nucleic acid molecule.
46. The gRNA of embodiment 38, wherein the editing event comprises introducing a splice site within the target nucleic acid molecule.
47. The gRNA of embodiment 38, wherein the editing event comprises disrupting a splice site within the target nucleic acid molecule.
48. The gRNA of any one of embodiments 38-47, wherein the target nucleic acid molecule comprises a chromosome or a genomic DNA molecule.
49. The gRNA of any one of embodiments 38-47, wherein the target nucleic acid molecule comprises the target domain.
50. The gRNA of embodiment 49, wherein the targeting domain of the gRNA base-pairs (in full or partial complementarity) with the sequence of the double-stranded target nucleic acid molecule that is complementary to the sequence of the target domain, which is the strand complementary to the strand that comprises a PAM sequence.
51. The gRNA of embodiment 50, wherein the targeting domain of the gRNA does not include the PAM sequence.
52. The gRNA of embodiment 50, wherein the location of the PAM may be 5′ or 3′ of the target domain sequence.
53. The gRNA of embodiment 51, wherein the position of the target nucleobases in the target domain is about 1, 2, 3, 4, 5, 6, 7, 8, 9, 10, 11, 12, 13, 14, 15, 16, 17, 18, 19, or 20 nucleobases from the PAM.
54. The gRNA of any one of embodiments 11-53, wherein the editing event reduces the activity of CD33 (Siglec-3) in a cell.
55. The gRNA of any one of embodiments 11-54, wherein the editing event reduces the expression level of a nucleic acid encoding CD33 (Siglec-3) in a cell.
56. The gRNA of any one of embodiments 11-55, wherein the editing event reduces the expression level of a CD33 (Siglec-3) protein in a cell.
57. The gRNA of any one of embodiments 11-56, wherein the editing event reduces or abolishes the expression of a full-length CD33 (Siglec-3) RNA or CD33 (Siglec-3) protein in a cell.
58. The gRNA of any one of embodiments 11-57, wherein the editing event reduces the activity of CLL-1 in a cell.
59. The gRNA of any one of embodiments 11-58, wherein the editing event reduces the expression level of a nucleic acid encoding CLL-1 in a cell.
60. The gRNA of any one of embodiments 11-59, wherein the editing event reduces the expression level of a CLL-1 protein in a cell.
61. The gRNA of any one of embodiments 11-60, wherein the editing event reduces or abolishes the expression of a full-length CLL-1 RNA or CLL-1 protein in a cell.
62. The gRNA of any one of embodiments 11-61, wherein the editing event reduces the activity of CD123 in a cell.
63. The gRNA of any one of embodiments 11-62, wherein the editing event reduces the expression level of a nucleic acid encoding CD123 in a cell
64. The gRNA of any one of embodiments 11-63, wherein the editing event reduces the expression level of a CD123 protein in a cell
65. The gRNA of any one of embodiments 11-64, wherein the editing event reduces or abolishes the expression of a full-length CD123 RNA or CD123 protein in a cell.
66. The gRNA of any one of embodiments 11-65, wherein the editing event reduces the activity of CD327 (Siglec-6) in a cell.
67. The gRNA of any one of embodiments 11-66, wherein the editing event reduces the expression level of a nucleic acid encoding CD327 (Siglec-6) in a cell.
68. The gRNA of any one of embodiments 11-67, wherein the editing event reduces the expression level of a CD327 (Siglec-6) protein in a cell.
69. The gRNA of any one of embodiments 11-68, wherein the editing event reduces or abolishes the expression of a full-length CD327 (Siglec-6) RNA or CD327 (Siglec-6) protein in a cell.
70. The gRNA of any one of embodiments 11-69, wherein the editing event reduces the activity of CD312 (EMR2) in a cell.
71. The gRNA of any one of embodiments 11-70, wherein the editing event reduces the expression level of a nucleic acid encoding CD312 (EMR2) in a cell
72. The gRNA of any one of embodiments 11-71, wherein the editing event reduces the expression level of a CD312 (EMR2) protein in a cell
73. The gRNA of any one of embodiments 11-72, wherein the editing event reduces or abolishes the expression of a full-length CD312 (EMR2) RNA or CD312 (EMR2) protein in a cell.
74. The gRNA of any one of embodiments 25-73, wherein the cell expresses a truncated version of a CD33 (Siglec-3), CLL-1, CD123, CD327 (Siglec-6), and/or CD312 (EMR2) RNA or protein.
75. The gRNA of embodiment 74, wherein the truncated version of the a CD33 (Siglec-3), CLL-1, CD123, CD327 (Siglec-6), and/or CD312 (EMR2) RNA or protein is expressed at a level equal to or greater than a level of a full-length a CD33 (Siglec-3), CLL-1, CD123, CD327 (Siglec-6), and/or CD312 (EMR2) RNA or protein in a non-edited cell.
76. The gRNA of embodiment 75, wherein a function or an activity of the truncated version of the a CD33 (Siglec-3), CLL-1, CD123, CD327 (Siglec-6), and/or CD312 (EMR2) RNA or protein is impaired or abolished.
77. The gRNA of embodiment 76, wherein the function or activity comprises binding to an antibody or a chimeric antigen receptor (CAR).
78. The gRNA of any one of the preceding embodiments, wherein the targeting domain is 16 nucleotides or more in length.
79. The gRNA of any one of the preceding embodiments, wherein the targeting domain is between about 16 to about 30 nucleotides in length.
80. The gRNA of any one of the preceding embodiments, wherein the targeting domain is 30 nucleotides in length.
81. The gRNA of any one of the preceding embodiments, wherein the targeting domain is 21 nucleotides in length.
82. The gRNA of any one of the preceding embodiments, wherein the targeting domain is 20 nucleotides in length.
83. The gRNA of any one of the preceding embodiments, wherein the targeting domain comprises a sequence of any one of SEQ ID NOs: 1-2021 or the reverse complement thereof, or a sequence having at least 90% or 95% identity thereto, or a sequence having no more than 1, 2, or 3 mutations relative thereto.
84. The gRNA of any one of the preceding embodiments, wherein the targeting domain comprises at least 16 consecutive nucleotides of any one of SEQ ID NOs: 1-2021, and/or base pairs or is complementary with at least 10 nucleotides of the target domain of any one of SEQ ID NOs: 1-2021. 85. The gRNA of any one of the preceding embodiments, which is a single guide RNA (sgRNA).
86. The gRNA of any one of the preceding embodiments, which comprises one or more chemical modifications.
87. The gRNA of any one of the preceding embodiments, which binds a base editor.
88. The gRNA of embodiment 87, wherein the base editor is a cytosine base editor (CBE).
89. The gRNA of embodiment 88, wherein the CBE is CBE1, CBE2, CBE3, or CBE4.
90. The gRNA of embodiment 88 or 89, wherein the CBE is selected from the group consisting of nCas9-2×UGI; BE4-rAPOBEC1; BE4-rAPOBEC1 K34A H122A; BE4-PpAPOBEC1; BE4-PpAPOBEC1 R33A; BE4-PpAPOBEC1 H122A; BE4-RrA3F; BE4-AmAPOBEC1; and BE4-SsAPOBEC3B.
91. The gRNA of any one of embodiments 88-90, wherein the CBE is a CBE-PpAPOBEC1 WT.
92. The gRNA of embodiment 87, wherein the base editor is an adenine base editor (ABE).
93. The gRNA of embodiment 92, wherein the ABE is ABE1, ABE2, ABE3, ABE4, ABE5, ABE6, ABE7, or ABE8.
94. The gRNA of embodiment 92 or 93, wherein the ABE is selected from the group consisting of ABE7.10-m; ABE7.10-d; ABE8.8-m; ABE8.8-d; ABE8.13-m; ABE8.13-d; ABE8.17-m; ABE8.17-d; ABE8.20-m; and ABE8.20-d.
95. The gRNA of any one of embodiments 92-94, wherein the ABE is an ABE8.
96. The gRNA of embodiment 87, wherein the base editor is a wildtype base editor.
97. A ribonucleoprotein (RNP) complex comprising a gRNA of any one of embodiments 1-96 and a base editor.
98. The gRNA of embodiment 97, wherein the base editor is a cytosine base editor (CBE).
99. The gRNA of embodiment 98, wherein the CBE is CBE1, CBE2, CBE3, or CBE4.
100. The gRNA of embodiment 98 or 99, wherein the CBE is selected from the group consisting of nCas9-2×UGI; BE4-rAPOBEC1; BE4-rAPOBEC1 K34A H122A; BE4-PpAPOBEC1; BE4-PpAPOBEC1 R33A; BE4-PpAPOBEC1 H122A; BE4-RrA3F; BE4-AmAPOBEC1; and BE4-SsAPOBEC3B.
101. The gRNA of embodiment 98, wherein the CBE is a CBE-PpAPOBEC1 WT.
102. The gRNA of embodiment 97, wherein the base editor is an adenine base editor (ABE).
103. The gRNA of embodiment 102, wherein the ABE is ABE1, ABE2, ABE3, ABE4, ABE5, ABE6, ABE7, or ABE8.
104. The gRNA of embodiment 102 or 103, wherein the ABE is selected from the group consisting of ABE7.10-m; ABE7.10-d; ABE8.8-m; ABE8.8-d; ABE8.13-m; ABE8.13-d; ABE8.17-m; ABE8.17-d; ABE8.20-m; and ABE8.20-d.
105. The gRNA of embodiment 102, wherein the ABE is an ABE8.
106. The gRNA of embodiment 97, wherein the base editor is a wildtype base editor.
107. A composition comprising a pre-formed complex comprising a base editor and a gRNA of any one of embodiments 1-96.
108. A mixture comprising an mRNA encoding a base editor and a gRNA of any one of embodiments 1-96.
109. A method for base editing, comprising:
   contacting a target domain in a double-stranded DNA molecule with a complex comprising a base editor and a guide RNA (gRNA) of any one of embodiments 1-96,
   wherein the base editor is a CBE or a ABE with a higher on-target editing efficiency as compared to a variant base editor.
110. The method of embodiment 109, wherein the base editor is a wildtype base editor.
111. The method of embodiment 110, wherein the wildtype base editor comprises BE4-PpAPOBEC.
112. The method of embodiment 109, wherein the variant base editor comprises BE4-PpAPOBEC1 R33A.
113. The method of any one of embodiments 109-112, wherein the double-stranded DNA molecule is in a cell.
114. The method of embodiment 113, which comprises contacting the cell with the gRNA and an mRNA that encodes the base editor.
115. The method of embodiment 114, wherein the mRNA that encodes the base editor is chemically modified to improve expression of the encoded base editor.
116. The method of 115, wherein the chemically modified mRNA comprises a 5-methoxyuridine modification.
117. The method of embodiment 115, wherein the chemically modified mRNA comprises a N1-methylpseudouridine modification.
118. The method of any one of embodiments 114-117, which comprises contacting the cell with a ribonucleoprotein (RNP) complex comprising the gRNA and the base editor.
119. A method for multiplex base editing, comprising:
   (i) providing a cell, and
   (ii) introducing into the cell
      (a) one or more guide RNAs (gRNAs) that target CD33 (Siglec-3), one or more gRNAs that target CLL-1, one or more gRNAs that target CD123, one or more gRNAs that target CD327 (Siglec-6), and/or one or more gRNAs that target CD312 (EMR2); and
      (b) a base editor that binds the one or more gRNAs,
   wherein the one or more gRNAs are configured to provide an editing event within the same or different target domains, thereby producing a genetically engineered cell.
120. A method of producing a genetically engineered cell, comprising:
   (i) providing a cell, and
   (ii) introducing into the cell
      (a) one or more guide RNAs (gRNAs) that target CD33 (Siglec-3), one or more gRNAs that target CLL-1, one or more gRNAs that target CD123, one or more gRNAs that target CD327 (Siglec-6), and/or one or more gRNAs that target CD312 (EMR2); and
      (b) a base editor that binds the one or more gRNAs,
   wherein the one or more gRNAs are configured to provide an editing event within the same or different target domains, thereby producing a genetically engineered cell.
121. A method for multiplex base editing, comprising:
   (i) providing a cell, and
   (ii) introducing into the cell
      (a) one or more guide RNAs (gRNAs) that target CD33;
      (b) one or more gRNAs that target CLL-1 and/or one or more gRNAs that target CD123; and
      (c) a base editor that binds the one or more gRNAs,
   wherein the one or more gRNAs are configured to provide an editing event within different target domains, thereby producing a genetically engineered cell.
122. A method of producing a genetically engineered cell, comprising:
   (i) providing a cell, and
   (ii) introducing into the cell
      (a) one or more guide RNAs (gRNAs) that target CD33;
      (b) one or more gRNAs that target CLL-1 and/or one or more gRNAs that target CD123; and
      (c) a base editor that binds the one or more gRNAs, wherein the one or more gRNAs are configured to provide an editing event within different target domains, thereby producing a genetically engineered cell.
123. A method for multiplex base editing, comprising:
   (i) providing a cell, and
   (ii) introducing into the cell
      (a) one or more guide RNAs (gRNAs) that target CD33 (Siglec-3);
      (b) one or more gRNAs that target CLL-1, one or more gRNAs that target CD123, one or more gRNAs that target CD327 (Siglec-6), and/or one or more gRNAs that target CD312 (EMR2); and
      (c) a base editor that binds the one or more gRNAs,
   wherein the one or more gRNAs are configured to provide an editing event within the same or different target domains, thereby producing a genetically engineered cell.
124. A method of producing a genetically engineered cell, comprising:
   (i) providing a cell, and
   (ii) introducing into the cell
      (a) one or more guide RNAs (gRNAs) that target CD33 (Siglec-3);
      (b) one or more gRNAs that target CLL-1, one or more gRNAs that target CD123, one or more gRNAs that target CD327 (Siglec-6), and/or one or more gRNAs that target CD312 (EMR2); and
   (c) a base editor that binds the one or more gRNAs,
wherein the one or more gRNAs are configured to provide an editing event within the same or different target domains, thereby producing a genetically engineered cell.
125. A method for triplex base editing, comprising:
   (i) providing a cell, and
   (ii) introducing into the cell
      (a) a plurality of gRNAs configured to provide simultaneous editing events within at least three different genomic targets; and
      (d) a base editor that binds the plurality of gRNAs, thereby producing a genetically engineered cell.
126. A method for triplex base editing, comprising:
   (i) providing a cell, and
   (ii) introducing into the cell
      (a) one or more gRNAs that target CD33 (Siglec-3);
      (b) one or more gRNAs that target CLL1;
      (c) one or more gRNAs that target CD123; and
   (d) a base editor that binds the one or more gRNAs,
wherein the one or more gRNAs are configured to provide simultaneous editing events within at least three different target domains, thereby producing a genetically engineered cell.
127. A method of producing a genetically engineered cell, comprising:
   (i) providing a cell, and
   (ii) introducing into the cell
      (a) a plurality of gRNAs configured to provide simultaneous editing events within at least three different genomic targets; and
      (d) a base editor that binds the plurality of gRNAs, thereby producing a genetically engineered cell.
128. A method of producing a genetically engineered cell, comprising:
   (i) providing a cell, and
   (ii) introducing into the cell
      (a) one or more gRNAs that target CD33 (Siglec-3);
      (b) one or more gRNAs that target CLL1;
      (c) one or more gRNAs that target CD123; and
      (d) a base editor that binds the one or more gRNAs,
   wherein the one or more gRNAs are configured to provide simultaneous editing events within at least three different target domains, thereby producing a genetically engineered cell.
129. A method for quadruplex base editing, comprising:
   (i) providing a cell, and
   (ii) introducing into the cell
      (a) a plurality of gRNAs configured to provide simultaneous editing events within at least four different genomic targets; and
      (d) a base editor that binds the plurality of gRNAs, thereby producing a genetically engineered cell.
130. A method for quadruplex base editing, comprising:
   (i) providing a cell, and
   (ii) introducing into the cell
      (a) one or more gRNAs that target CD33 (Siglec-3);
      (b) one or more gRNAs that target CLL1;
      (c) one or more gRNAs that target CD123;
      (d) one or more gRNAs that target CD312 (EMR2);
      (e) a base editor that binds the one or more gRNAs,
   wherein the one or more gRNAs are configured to provide simultaneous editing events within at least four different target domains, thereby producing a genetically engineered cell.
131. A method of producing a genetically engineered cell, comprising:
   (i) providing a cell, and
   (ii) introducing into the cell
      (a) a plurality of gRNAs configured to provide simultaneous editing events within at least four different genomic targets; and
      (b) a base editor that binds the plurality of gRNAs, thereby producing a genetically engineered cell.
132. A method of producing a genetically engineered cell, comprising:
   (i) providing a cell, and
   (ii) introducing into the cell
      (a) one or more gRNAs that target CD33 (Siglec-3);
      (b) one or more gRNAs that target CLL1;
      (c) one or more gRNAs that target CD123;
      (d) one or more gRNAs that target CD312 (EMR2);
      (e) a base editor that binds the one or more gRNAs,
   wherein the one or more gRNAs are configured to provide simultaneous editing events within at least four different target domains, thereby producing a genetically engineered cell.
133. The method of any one of the preceding embodiments, wherein the one or more guide RNAs (gRNAs) comprise a gRNA of any one of embodiments 1-96.
134. The method of any one of the preceding embodiments, which results in the concurrent editing of one or more target domains within the same gene and/or within different genes.
135. The method of any one of the preceding embodiments, which results in the concurrent editing of two or more target domains within the same gene and/or within different genes.
136. The method of any one of the preceding embodiments, which results in the concurrent editing of three or more target domains within the same gene and/or within different genes.
137. The method of any one of the preceding embodiments, which results in the concurrent editing of four or more target domains within the same gene and/or within different genes.
138. The method of any one of the preceding embodiments, which results in the concurrent editing of one or more target domains within a CD33 (Siglec-3) gene, a CLL-1 gene, a CD123 gene, a CD327 (Siglec-6) gene, and/or a CD312 (EMR2) gene.
139. The method of any one of the preceding embodiments, wherein the one or more gRNAs that target CD33 (Siglec-3) are designed for use with a cytosine base editor (CBE) and/or an adenine base editor (ABE)
140. The method of any one of the preceding embodiments, wherein the one or more gRNAs that target CD33 (Siglec-3) are designed for use with a CBE.
141. The method of any one of the preceding embodiments, wherein the one or more gRNAs that target CD33 (Siglec-3) are designed for use with a ABE.
142. The method of any one of the preceding embodiments, wherein the one or more gRNAs that target CLL1 are designed for use with a cytosine base editor (CBE) and/or an adenine base editor (ABE).
143. The method of any one of the preceding embodiments, wherein the one or more gRNAs that target CLL1 are designed for use with a CBE.
144. The method of any one of the preceding embodiments, wherein the one or more gRNAs that target CLL1 are designed for use with a ABE.
145. The method of any one of the preceding embodiments, wherein the one or more gRNAs that target CD123 are designed for use with a CBE and/or an ABE.
146. The method of any one of the preceding embodiments, wherein the one or more gRNAs that target CD123 are designed for use with a CBE.
147. The method of any one of the preceding embodiments, wherein the one or more gRNAs that target CD123 are designed for use with a ABE.
148. The method of any one of the preceding embodiments, wherein the one or more gRNAs that target EMR2 are designed for use with a CBE and/or an ABE.
149. The method of any one of the preceding embodiments, wherein the one or more gRNAs that target EMR2 are designed for use with a CBE.
150. The method of any one of the preceding embodiments, wherein the one or more gRNAs that target EMR2 are designed for use with a ABE.
151. The method of any one of the preceding embodiments, which comprises contacting the cell with the one or more gRNAs and an mRNA that encodes the base editor.
152. The method of any one of the preceding embodiments, which comprises contacting the cell with a ribonucleoprotein (RNP) complex comprising the one or more gRNAs and the base editor.
153. The method of any one of the preceding embodiments, which comprises contacting the cell with the gRNA and an mRNA that encodes the base editor.
154. The method of any one of the preceding embodiments, wherein the mRNA that encodes the base editor is chemically modified to improve expression of the encoded base editor.
155. The method of any one of the preceding embodiments, wherein the chemically modified mRNA comprises a 5-methoxyuridine modification.
156. The method of any one of the preceding embodiments, wherein the chemically modified mRNA comprises a N1-methylpseudouridine modification.
157. The method of any one of the preceding embodiments, wherein the RNP is introduced into the cell via electroporation.
158. The method of any one of the preceding embodiments, wherein the base editor is a wildtype base editor.
159. The method of any one of the preceding embodiments, wherein the base editor is a cytosine base editor (CBE) and/or an adenine base editor (ABE)
160. The method of any one of the preceding embodiments, wherein only a CBE is introduced into the cell.
161. The method of any one of the preceding embodiments, wherein only an ABE is introduced into the cell.
162. The method of any one of the preceding embodiments, wherein both a CBE and an ABE are introduced into the cell.
163. The method of any one of the preceding embodiments, wherein a wildtype base editor is introduced into the cell, optionally, wherein a wildtype base editor targets a cytosine-guanine (C-G) base pair or a thymine-adenine (T-A) base pair with higher on-target editing efficiency as compared to a variant base editor.
164. The method of any one of the preceding embodiments, which results in a lower translocation risk as compared to a variant base editor, optionally, wherein the method results in 0% translocations, or an undetectable level of translocations, and an on-target editing efficiency of at least about 25%, at least about 30%, at least about 35%, at least about 40%, at least about 45%, at least about 50%, at least about 55%, at least about 60%, at least about
65%, at least about 70%, at least about 75%, at least about 80%, at least about 85%, at least about 90%, at least about 95%, or at least about 99% or more for a modification in the endogenous CD33 (Siglec-3), CLL-1, CD123, CD327 (Siglec-6), and/or CD312 (EMR2) gene.
167. The method of any one of the preceding embodiments, wherein the cell comprises a hematopoietic stem cell or a progenitor cell.
168. A genetically engineered hematopoietic stem or progenitor cell, which is produced by a method of The method of any one of the preceding embodiments.
169. A cell population, comprising a plurality of the genetically engineered hematopoietic stem or progenitor cells of embodiment 168.
170. A cell population comprising a plurality of the genetically engineered hematopoietic stem or progenitor cells, wherein at least a portion of the cells comprise:
   (i) an edited CD33 (Siglec-3) gene;
   (ii) an edited CLL-1 gene;
   (iii) an edited CD123 gene;
   (iv) an edited CD327 (Siglec-6) gene;
   (v) an edited CD312 (EMR2) gene;
   (vi) an edited CD33 (Siglec-3) gene and an edited CLL-1 gene;
   (vii) an edited CD33 (Siglec-3) gene and an edited CD123 gene;
   (viii) an edited CD33 (Siglec-3) gene and an edited CD327 (Siglec-6) gene;
   (ix) an edited CD33 (Siglec-3) gene and an edited CD312 (EMR2) gene;
   (x) an edited CD33 (Siglec-3) gene, an edited CLL-1 gene, and an edited CD123 gene;
   (xi) an edited CD33 (Siglec-3) gene, an edited CLL-1 gene, an edited CD123 gene, and an edited CD327 (Siglec-6) gene;
   (xii) an edited CD33 (Siglec-3) gene, an edited CLL-1 gene, an edited CD123 gene, an edited CD327 (Siglec-6) gene, and an edited CD312 (EMR2) gene; or
   (xiii) an edited CD33 (Siglec-3) gene, an edited CLL-1 gene, an edited CD123 gene, an edited CD327 (Siglec-6) gene, and/or an edited CD312 (EMR2) gene.
180. The cell population of any one of the preceding embodiments, wherein a CD33 (Siglec-3) gene comprises a stop codon or a mutated splice site, but not a frameshift mutation which is typically introduced by CRISPR nuclease-mediated nonhomologous end joining (NHEJ).
181. The cell population of any one of the preceding embodiments, wherein a CLL-1 gene comprises a stop codon or a mutated splice site, but not a frameshift mutation which is typically introduced by CRISPR nuclease-mediated nonhomologous end joining (NHEJ).
182. The cell population of any one of the preceding embodiments, wherein a CD123 gene comprises a stop codon or a mutated splice site, but not a frameshift mutation which is typically introduced by CRISPR nuclease-mediated nonhomologous end joining (NHEJ).
183. The cell population of any one of the preceding embodiments, wherein a CD327 (Siglec-6) gene comprises a stop codon or a mutated splice site, but not a frameshift mutation which is typically introduced by CRISPR nuclease-mediated nonhomologous end joining (NHEJ).
184. The cell population of any one of the preceding embodiments, wherein a CD312 (EMR2) gene comprises a stop codon or a mutated splice site, but not a frameshift mutation which is typically introduced by CRISPR nuclease-mediated nonhomologous end joining (NHEJ).
185. The cell population of any one of the preceding embodiments, which expresses less than 30% of the CD33 (Siglec-3) expressed by a wild-type counterpart cell population.
186. The cell population of any one of the preceding embodiments, which expresses less than 30% of the CLL-1 expressed by a wild-type counterpart cell population.
187. The cell population of any one of the preceding embodiments, which expresses less than 30% of the CD123 expressed by a wild-type counterpart cell population.
188. The cell population of any one of the preceding embodiments, which expresses less than 30% of the CD327 (Siglec-6) expressed by a wild-type counterpart cell population.
189. The cell population of any one of the preceding embodiments, which expresses less than 30% of the CD312 (EMR2) expressed by a wild-type counterpart cell population.
190. The cell population of any one of the preceding embodiments, wherein at least a portion of the cells have genetic editing at a gene encoding a lineage-specific cell-surface antigen other than CD33 (Siglec-3), CLL-1, CD123, CD327 (Siglec-6), or CD312 (EMR2).
191. The cell population of embodiment 190, wherein the gene encoding a lineage-specific cell surface antigen other than CD33 (Siglec-3), CLL-1, CD123, CD327 (Siglec-6), or CD312 (EMR2) is CD19, CD30, CD5, CD6, CD7, CD34, CD38, or BCMA.
192. A method, comprising administering to a subject in need thereof a cell population of any one of the preceding embodiments, optionally wherein the subject has a hematopoietic malignancy.
193. The method of any one of the preceding embodiments, wherein the hematopoietic malignancy comprises Hodgkin lymphoma, non-Hodgkin lymphoma, leukemia, or multiple myeloma.
194. The method of any one of the preceding embodiments, wherein the leukemia comprises acute myeloid leukemia (AML), acute lymphoid leukemia, chronic myelogenous leukemia, acute lymphoblastic leukemia or chronic lymphoblastic leukemia, and chronic lymphoid leukemia.
195. The method of any one of the preceding embodiments, wherein the hematopoietic malignancy comprises acute myeloid leukemia (AML).
196. The method of any one of the preceding embodiments, which further comprises administering to the subject an effective amount of an agent that targets CD33 (Siglec-3), CLL-1, CD123, CD327 (Siglec-6), and/or CD312 (EMR2), wherein the agent comprises an antigen binding fragment that binds CD33 (Siglec-3), CLL-1, CD123, CD327 (Siglec-6), and/or CD312 (EMR2).
197. The method of any one of the preceding embodiments, wherein the agent that targets CD33 (Siglec-3), CLL-1, CD123, CD327 (Siglec-6), and/or CD312 (EMR2) is an antibody or a chimeric antigen receptor (CAR).
198. A nucleic acid encoding the gRNA of any one of embodiments 1-96.
199. A kit or composition comprising: a) a gRNA of any one of embodiments 1-96, or a nucleic acid encoding the gRNA, and b) a second gRNA, or a nucleic acid encoding the second gRNA.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1A is a schematic showing an example of precise genome editing using a cytosine base editor (CBE) and an adenine base editor (ABE).

FIG. 1B is a schematic showing exemplary base editing applications for gene silencing by the introduction of a STOP codon (top right panel), multiplex editing with reduced translocations and low off-target activity (bottom right panel), gene correction (top left panel), and epitope engineering (bottom left panel).

FIG. 1C shows an exemplary multiplex editing strategy to identify and nominate base editing guides to target therapeutic genes including, for example, CD33 and CLL-1.

FIG. 2A shows an exemplary in silico base editors guide design and prioritization, which can be used to achieve gene knockout (KO) using base editors via the introduction of a premature STOP codon or splice site disruption.

FIG. 2B shows exemplary CD33 and CLL-1 base editor candidate guides to induce protein knockout (KO).

FIG. 2C is a schematic showing exemplary base editor CD33 and CLL-1 guides binding sites.

FIG. 3A shows the selecting of exemplary CBE and ABE constructs with high on-target activity and reduced off-target activity. Adapted from Yu et al., Cytosine base editors with minimized unguided DNA and RNA off-target events and high on-target activity. Nature Communications, Volume 11, Article number 2052 (2020); and Gaudelli et al., Directed evolution of adenine base editors with increased activity and therapeutic application. Nature Biotechnology Volume 38, pages 892-900 (2020), the entire contents of each of which are incorporated herein by reference.

FIG. 3B shows exemplary base editor delivery in hematopoietic stem and progenitor cells (HSPCs) using mRNA with chemical modifications.

FIG. 3C shows an exemplary engineering protocol to edit HSPCs using base editors.

FIG. 3D shows an exemplary CD33 CBE Guide Screen demonstrating high on-target base editing in HSPCs using guides 7, 8, and 17. N1: N1-methylpseudouridine-modified mRNA encoding the base editor; 5-mO: 5-methoxyuridine-modified mRNA encoding the base editor.

FIG. 3E shows an exemplary CLL-1 CBE Guide Screen demonstrating high on-target base editing in HSPCs using guides 3 and 4.

FIG. 4A shows an exemplary CD33/CLL-1 protein knockout (KO) experimental plan.

FIG. 4B shows a study summary for assess CD33/CLL-1 protein KO using exemplary base editor guides, and comparing CBE WT and R33A variants.

FIG. 4C shows that cytosine/adenine base editing of CD33E1-Splice Site using different guide RNAsefficiently disrupts CD33 expression. Inset shows results for guide RNA 17. EP: electroporation.

FIG. 4D shows an exemplary nonsense mediated decay mechanism postulated to mediate protein KO when CD33E1 splice donor site is disrupted by CBE/ABE—CD33sg17.

FIG. 4E shows cytosine base editing of CLL-1 using various guide RNAs efficiently disrupts protein expression. Inset shows results for guide RNA 3.

FIG. 4F shows base editor technology improvements on gene KO potential. These data demonstrate that exemplary CD33 BE and guide combinations can achieve CD33 protein loss in HSPCs or more than 60% CD33, and that exemplary CLL-1 BE and guide combinations can achieve more than 60% CLL-1 protein loss in HPSCs.

FIG. 5A is a schematic showing exemplary criteria to select and prioritize base editor guides.

FIG. 5B shows base editing enables efficient CD33 protein KO using targeting iSTOP guides 7 and 8 and SpliceR guide 17—Guide Screen 2 CBE_R33A N1mod.

FIG. 5C shows CD33 CBE (and ABEsg17) Guide Screen shows high on-target base editing in HSPCs using guides 7, 8, and 17.

FIG. 5D shows cell viability and cell growth CD33 Guide Screen 2 results.

FIG. 5E shows cell viability and cell growth CLL-1 Guide Screen 2 results.

FIG. 5F shows cell viability CD33/CLL-1—Constructs comparison results.

FIG. 5G shows cell growth CD33/CLL-1—Constructs comparison results.

FIG. 5H shows CLL-1 protein KO time course.

FIG. 6A shows an exemplary CBE CD33+CLL-1 multiplex base editing and experimental plan.

FIG. 6B shows summary of study arm for dose titration multiplex base editing top CBE CD33 guides with top CLL-1 guide.

FIG. 6C shows multiplex base edited CD34+ cells show efficient cell surface CD33 and CLL-1 protein KO in HPSCs.

FIG. 6D shows multiplex base edited CD34+ cells show efficient CD33 and CLL-1 protein KO in HPSCs.

FIG. 6E shows CD33 protein KO data in HPSCs. Data for CD33g8 and CLL-1g3 are highlighted. Exemplary off-target analysis is shown on the bottom left for CD33sg8, demonstrating a desirable off-target profile.

FIG. 6F shows flow data demonstrating that ˜80% of multiplex edited cells lack CD33 and CLL-1 surface protein expression.

FIG. 7 shows data demonstrating that base editing does not impact cell differentiation of HPSCs.

FIG. 8A shows comboplexing-simultaneous delivery of Cytosine Base Editor and Cpf1 nuclease allows for single delivery and no translocation risk as BE does not make double strand break.

FIG. 8B shows viability and cell growth is not impacted when delivering simultaneously CBE and AsCpf1 in CD34 cells.

FIG. 9A is a schematic showing the experimental design for multiplex editing of CD33 and CLL-1 performed using different CD33 guide RNAs (sg7, sg8, or sg17), in combination with CLL-1 guide RNA sg3.

FIG. 9B shows myeloid in vitro differentiation data assessed by Flow Cytometry for protein knockout (KO) readout. The base editor (BE) combination of CD33g8 and CLL-1g3 showed 80% double surface protein KO.

FIG. 9C shows that balanced translocations were not detected in the multiplex base edited samples as determined by a RhampSeq assay.

FIG. 10 is a schematic showing Multiplex Base Editing using Cytosine Base Editors (CBEs) in CD34+ hematopoietic stem and progenitor cells (HSPCs).

FIG. 11A shows mapping of stop codon and splice disrupter base editing guides on the CD33 locus and Cas9 control binding sites (left panel), and on-target base editing efficiency of top three CBE gene KO inducing single guides (sg7, sg8, or sg17, labelled g7, g8, or g17) using three different CBE4 mRNA encoding constructs (right panel). The gray bar represents Cas9-induced indel frequency on the CD33 locus.

FIG. 11B shows mapping of stop codon and splice disrupter base editing guides on the CLL-1 locus and Cas9 control binding sites (left panel), and on-target base editing efficiency of top two CBE gene KO inducing single guides (sg3 or sg4, labelled g3 or g4) using three different CBE4 mRNA encoding constructs (right panel). The gray bar represents Cas9-induced indel frequency on the CLL-1 locus.

FIG. 12 shows CD33 (left panel) and CLL-1 (right panel) surface protein expression quantified in edited CD34+ HSPCs by flow cytometry 9 days post-electroporation (EP) for 3 different CD33 gRNAs (sg7, sg8, or sg17, labelled g7, g8, or g17, left panel) or two different CLL-1 gRNAs (sg3 or sg4, labelled g3 or g4). Histograms are color coded per guide and show percentage of positive population for CD33 or CLL-1 edited samples compared to control untreated cells.

FIG. 13A shows on-target editing efficiency of single and multiplex base edited cells for CD33 and CLL-1 compared to multiplex Cas9 control edited cells.

FIG. 13B shows normalized CD33 and CLL-1 surface protein expression in edited and unedited CD34+ HSPCs using flow cytometry 9 days post-EP in myeloid differentiating culture conditions.

FIG. 14 shows edited and untreated control HSPCs that were cultured in differentiating culture conditions to support growth of multiple progenitor cell lineages. Colony-forming unit (CFUs) were measured 14 days after plating and Erythroid (BFU-E), Myeloid (CFU-G/M/GM) and Mixed (CFU-GEMM) lineages were quantified.

FIG. 15A shows CD34+ HSPCs that were electroporated with CBE4 encoding mRNA or Cas9 ribonucleoprotein (RNP) complex using CD33 and CLL-1 synthetic guides and in vitro differentiated into monocytic lineage.

FIG. 15B shows on-target editing efficiency of CD33 and CLL-1 in base edited and Cas9-edited samples harvested at different time points post-EP (Day 2) and throughout monocytic differentiation. Editing efficiency was calculated and annotated using CRISPResso v2.0.30 and variant effector predictor (VEP). CD33 (top panel) and CLL-1 (bottom panel) protein expression in edited and unedited samples were measured throughout monocytic differentiation using flow cytometry. Bulk population throughout multiplex base edited cells showed a decrease in CD33 and CLL-1 expression in monocyte differentiated CD34+ HSPCs.

FIG. 16A shows frequency of On-On (CD33-CLL-1 cut site) translocation events using a multiplex rhAmpSeq approach with coverage of 217442 collapsed and aligned reads to a 223 bp junction of the expected translocation between the two different loci.

FIG. 16B shows representative metaphase spread using a directional genomic hybridization (dGH) assay in edited and unedited samples showing chromosomal paints in pink (chromosomes 1, 2, 3) used as normalizers to account for donor variability (dosimetry); yellow (chromosome 12, CLL-1 locus) and green (chromosome 19, CD33 locus).

FIG. 17A shows comboplexing-simultaneous delivery of adenine base editor (ABE) and gRNA targeting CD33 and CD123 (e.g., CD33g17 and CD123g18, respectively) allows for about 90% on-target editing efficiency in CD123.

FIG. 17B shows off-target profile of adenine base editing with CD123g18.

FIG. 17C is a schematic showing the experimental design for multiplex editing of CD33 and CD123 performed using different CD33 and CD123 guide RNAs in combination with an adenine base editor (ABE).

FIG. 18A shows on-target editing efficiency of single base edited cells for CD33 compared to Cas9 control edited cells. Base editing was performed using different CD33 guide RNAs (e.g., CD33g7, CD33g8, and CD33g17) in combination with an adenine base editor (ABE) or a cytosine base editor (CBE). N1: N1-methylpseudouridine-modified mRNA encoding the base editor; 5-mO: 5-methoxyuridine-modified mRNA encoding the base editor. The combination of adenine base editor (ABE) and CD33g17 resulted in about 95% on-target editing efficiency at 120 hours post electroporation (EP) of the CD34+ HSPCs with 9 μg of 5-methoxyuridine-modified mRNA encoding the adenine base editor (ABE).

FIG. 18B shows on-target editing efficiency of single base edited cells for CD33 performed using different CD33 guide RNAs (e.g., CD33g7, CD33g8, and CD33g17) in combination with an adenine base editor (ABE) or a cytosine base editor (CBE). N1: N1-methylpseudouridine-modified mRNA encoding the base editor; 5-mO: 5-methoxyuridine-modified mRNA encoding the base editor. The combination of adenine base editor (ABE) and CD33g17 resulted in substantially all edits creating a substitution that would disrupt splicing at 120 hours post electroporation (EP) of the CD34+ HSPCs with 9 μg of 5-methoxyuridine-modified mRNA encoding the adenine base editor (ABE).

FIG. 18C shows CD33 surface protein expression in edited and unedited CD34+ HSPCs 120 hours post electroporation (EP). The combination of adenine base editor (ABE) and CD33g17 resulted in a strong loss of CD33 surface protein expression compared to unedited (MockEP) at 120 hours post electroporation (EP) of the CD34+ HSPCs with 9 μq of 5-methoxyuridine-modified mRNA encoding the adenine base editor (ABE).

FIG. 19 is a schematic showing the experimental design for adenine base editor (ABE) multiplex editing. CD34+ cells are thawed and allowed to rest in culture for 48 hours. Then, for the ABE portion (boxed), ABE CD33g17 is paired for multiplex editing with each of the following guide RNAs: ABE CD123g17, ABE CD123g18, and ABE CD123g21. The two guide RNAs and mRNA encoding the adenine base editor (ABE) is electroporated into the cells. The cells are then cultured in Myeloid in vitro differentiation media. Flow cytometry was performed at day 2 and day 9 post-electroporation to measure surface protein expression of CD33 and CD123 in guide-edited cells samples using a cytometer, and cells for gDNA molecular analysis were collected on day 6 and day 9 post electroporation.

FIG. 20A shows comboplexing-simultaneous delivery of adenine base editor (ABE) and gRNA targeting CD33 and CD123 (e.g., CD33g17 and CD123g18, respectively) allows for about 90% on-target editing efficiency in CD123.

FIG. 20B shows percentage chimerism (right panel) and on-target editing input (right panel) post 16-week engraftment in bone marrow (BM) of no electroporation control (No EP), mock Electroporation control (Mock EP Ct1), single ABE gRNA targeting of CD33 or CD123 (e.g., using CD33g17 or CD123g18, respectively), and comboplexing-simultaneous ABE targeting of both CD33 and CD123 (e.g., using both CD33g17 and CD123g18). These data show that multiplex deletion of myeloid antigens by base editing in human hematopoietic stem and progenitor cells (HSPCs) enables potential for next generation transplant for acute myeloid leukemia (AML) treatment.

FIG. 20C shows splice site disruption frequencies induced by ABE increased consistently across the different arms of the study.

FIG. 21A is a schematic showing the experimental design for an in vivo study, Viivs 042, to assess persistence of editing and long-term reconstitution of simultaneously CBE CD33+CLL1 and ABE CD33+CD123 multiplex edited CD34+ HSPCs in NSG mice.

FIG. 21B shows the arms of the Viivs 042 study and material generation.

FIG. 22A shows is a schematic showing the experimental design for Viivs 042 input material generation workflow.

FIG. 22B is a table showing exemplary editing condition details for Viivs 042 study.

FIG. 23A shows cell viability for Viivs 042 study. These data show high viability (˜90%) between BE single and multiplex conditions.

FIG. 23B shows cell growth for Viivs 042 study. These data show similar cell counts between BE single and multiplex conditions.

FIG. 24A shows total editing efficiency for Viivs 042 study conditions.

FIG. 24B shows base editing efficiency for Viivs 042 study conditions. These data demonstrate that base editing efficiency in samples harvested 48 hour post electroporation (EP) for dosing showed expected alleles containing alleles with stop codons gain and splice sites disrupted.

FIG. 25 shows total editing efficiency for Viivs 042 study conditions. High total editing was confirmed in all samples 48 hours post EP (dosed cells) and a slight increase 144 hour post EP.

FIG. 26A shows colony-forming unit (CFU) results at 200 dilution.

FIG. 26B shows colony-forming unit (CFU) results at 400 dilution.

FIG. 27 shows percentage chimerism post 16-week engraftment in bone marrow (BM). These data demonstrate no impact in the chimerism in edited groups.

FIGS. 28A-28C shows highly efficient knockout of CD33 (FIG. 28A), CLL-1 (FIG. 28B), and CD123 (FIG. 28C) in edited groups with ABE.

FIGS. 29A-29H show no effect in lineage reconstitution in edited groups. FIG. 29A shows total lineage reconstitution in edited groups. FIGS. 29B-29H show lineage reconstitution in edited groups across different cell types, including: B-lymphocytes (FIG. 29B), T-lymphocytes (FIG. 29C), Monocytes (FIG. 29D), HSPCs (FIG. 29E), Granulocytes (FIG. 29F), cDCs (FIG. 29G), and pDCs (FIG. 29H).

FIGS. 30A-30E show high levels of CD123 KO in myeloid subpopulations across different cell types, including: Monocytes (FIG. 30A), Granulocytes (FIG. 30B), Mast/Basophils (FIG. 30C), cDCs (FIG. 30D), and pDCs (FIG. 30E).

FIGS. 31A-31E show low levels of double KO in myeloid subpopulations due to low levels of CLL1 KO across different cell types, including: Monocytes (FIG. 31A), Granulocytes (FIG. 31B), Mast/Basophils (FIG. 31C), cDCs (FIG. 31D), and pDCs (FIG. 31E).

FIG. 32 shows on-target editing analysis in Bone Marrow material across the different arms of the study. These data confirm editing persistence.

FIG. 33 shows stop codon frequencies induced by CBE slightly decreased consistently across the different arms of the study.

FIG. 34 shows splice site disruption frequencies induced by ABE increased consistently across the different arms of the study.

FIG. 35 is a schematic showing the characterization of BE Multiplex Scale-up for in vivo.

FIG. 36 is a schematic showing the various conditions assessed in the BE Multiplex Scale-up for in vivo.

FIG. 37 shows experimental conditions for the BE Multiplex Scale-up for in vivo.

FIGS. 38A-38B show cells counts and viability, respectively, for the BE Multiplex Scale-up for in vivo. Cells growth slightly reduced in the 6M cell, 2×Dose condition.

FIG. 39 shows flow gating strategy for the BE Multiplex Scale-up for in vivo.

FIG. 40A-40B shows flow cytometry data for CD33 and CLL-1, respectively.

FIG. 41 shows dual knockout of CD33 and CLL-1.

FIG. 42 shows base editing efficiency with CBE CD33sg8. These data demonstrate that 2× dose results in higher frequency of alleles that result in premature stop codon formation for CBE CD33g8.

FIG. 43 shows base editing efficiency with CBE CLL1g3. These data demonstrate that 2× dose results in higher frequency of alleles that result in premature stop codon formation for CBE CLL1g3.

FIG. 44 is a schematic showing experimental conditions for CBE and ABE editing of EMR2 and CD33.

FIG. 45 is a schematic showing CD33 and EMR2 guide screen landscape.

FIG. 46 is a schematic showing experimental plan for EMR2 guide screen and protein KO assessment.

FIGS. 47A-47B shows cell viability and cells counts, respectively, for CBE and ABE editing of EMR2 and CD33.

FIGS. 48A-48B each show reduced surface expression of EMR2. These data demonstrate that ABE EMR2 guides show strong protein KO 6 days post EP.

FIGS. 49A-49B each show that EMR2 experimental conditions resulted in varying levels of protein KO 6 days post EP.

FIGS. 50A-50B each show reduced surface expression of CD33. These data demonstrate that ABE CD33 guides show strong protein KO 6 days post EP.

FIGS. 51A-51B each show that CD33 experimental conditions resulted in varying levels of protein KO 6 days post EP.

FIGS. 52A-52B shows total editing efficiency and base editing efficiency with ABE, respectively. The ABE guide screen in HSPCs showed high editing in various sites of CD33 and EMR2 loci and low frequencies of bystander edits. All experimental conditions showed good viability (90%) and cell expansion compared to the MockEP control.

FIG. 53 shows editing efficiency of ABE CD33 gRNA.

FIG. 54 shows editing efficiency of ABE and CBE EMR2 gRNAs.

FIG. 55 is a schematic showing EMR2/CD33 Multiplex ABE Base-Editing.

FIG. 56 shows exemplary EMR2/CD33 Multiplex ABE Base-Editing conditions.

FIG. 57 shows ABE guides potential in silico off-target site.

FIGS. 58A-58B show cells counts and cell viability, respectively, for ABE editing of EMR2 and CD33.

FIGS. 59A-59B show ABE EMR2 and CD33 DNA editing frequency, respectively.

FIGS. 60A-60C show ABE EMR2 editing frequency, editing consequences, and base editing summary, respectively.

FIGS. 61A-61B show frequency of EMR2 Off-Target Editing in CD97 and consequences thereof, respectively.

FIGS. 62A-62C show ABE CD33 editing frequency, editing consequences, and base editing summary, respectively.

FIG. 63 shows EMR2 surface protein expression.

FIG. 64 shows EMR2 surface protein expression.

FIG. 65 shows CD33 surface protein expression.

FIG. 66 shows CD33 surface protein expression.

FIG. 67 is a schematic showing CBE single edits and quadruplex edits for CD33, CLL1, CD123, and EMR2.

FIG. 68 is a schematic showing experimental design to deliver CBE into CD34+ cells to target 4 loci simultaneously.

FIGS. 69A-69B show cell viability and cells counts, respectively, for CBE single edits and quadruplex edits for CD33, CLL1, CD123, and EMR2. These date demonstrate that quadruplex editing does not impact cell health.

FIG. 70 shows total editing efficiency for CBE single edits and quadruplex edits for CD33, CLL1, CD123, and EMR2. These data demonstrate that alleles multiplex deletion of myeloid antigens by base editing in human hematopoietic stem and progenitor cells (HSPCs) enables potential for next generation transplant for acute myeloid leukemia (AML) treatment.

FIG. 71 shows editing efficiency for CBE single edits and quadruplex edits for CD33, CLL1, CD123, and EMR2. These data demonstrate that alleles multiplex deletion of myeloid antigens by base editing in human hematopoietic stem and progenitor cells (HSPCs) enables potential for next generation transplant for acute myeloid leukemia (AML) treatment.

FIG. 72 is a schematic showing ABE CD33/CD123/EMR2 triple KO.

FIG. 73 shows exemplary electroporation conditions for ABE CD33/CD123/EMR2 triple KO.

FIGS. 74A-74B show DNA editing frequency on day 2 and day 5 post EP, respectively. These data demonstrate >80% editing for CD33 g16 and >90% editing for CD123 g18, EMR2 sDex13 and EMR2 sDex19 at day 5 post EP. Similar editing was observed for CD123 g18, EMR2 sDex13 and EMR2 sDex19 in single and Triplex EP condition. A slight decrease in editing for CD33 g16 in Triplex compared to single EP (˜5% decrease) was also observed. Higher editing was observed at Day 5 compared to Day 2 across all guides and conditions. No off-target editing in CD97 for EMR2 sDex13 and EMR2 sDex19 was observed.

FIGS. 75A-75B show DNA editing frequency on day 2 and day 5 post EP, respectively. These data demonstrate that the majority of editing for all guides in single and triplex conditions causes splice site disruption. EMR2 sDex13 shows ˜4% INDEL formation at Day 2 and Day 5 post-EP.

FIGS. 76A-76B are schematics showing detailed substitution percentage summary in CD33sg16 and CD123sg18 groups, respectively.

FIGS. 77A-77B are schematics showing detailed substitution percentage summary in EMR2sg13 and EMR2sg19 groups, respectively.

FIGS. 78A-78C are schematics showing flow cytometry gating strategy.

FIGS. 79A-79B show EMR2 surface protein expression and total gMFI, respectively.

FIGS. 80-81 show CD33 surface protein expression and total gMFI, respectively.

FIGS. 82A-82B show CD123 surface protein expression and total gMFI, respectively.

FIGS. 83A-83B show CD33, CD123, and EMR2 surface protein expression, and Triple KO surface expression, respectively.

FIGS. 84A-84B show CD33, CD123, and EMR2 DNA editing, and Triple KO surface protein analysis, respectively.

FIG. 85A shows CLL-1 ABE Guides with SpCas9 NGG PAM.

FIG. 85B shows CLL-1 ABE Guides with Relaxed PAM (NG).

FIG. 85C shows CLL-1 ABE Guides with Relaxed PAM (NRG).

FIG. 85D shows CLL-1 ABE Guides with Cpf1 TTTN PAM.

FIG. 85E is a schematic showing a CLL-1 gene overview.

FIG. 86 shows ABE/CBE g17 alignment to Siglec-6 predicting that g17 likely targets and disrupts both Siglec-3 and Siglec-6.

FIG. 87 shows Siglec-6 surface expression is decreased after editing with CD33g17.

### DETAILED DESCRIPTION

### Definitions

The term “binds”, as used herein with reference to a gRNA interaction with a target domain, refers to the gRNA molecule and the target domain forming a complex. The complex may comprise two strands forming a duplex structure, or three or more strands forming a multi-stranded complex. The binding may constitute a step in a more extensive process, such as the cleavage of the target domain by a Cas endonuclease. In some embodiments, the gRNA binds to the target domain with perfect complementarity, and in other embodiments, the gRNA binds to the target domain with partial complementarity, e.g., with one or more mismatches. In some embodiments, when a gRNA binds to a target domain, the full targeting domain of the gRNA base pairs with the targeting domain. In other embodiments, only a portion of the target domain and/or only a portion of the targeting domain base pairs with the other. In an embodiment, the interaction is sufficient to mediate a target domain-mediated cleavage event.

A “Cas9 molecule” as that term is used herein, refers to a molecule or polypeptide that can interact with a gRNA and, in concert with the gRNA, home or localize to a site which comprises a target domain. Cas9 molecules include naturally occurring Cas9 molecules and engineered, altered, or modified Cas9 molecules that differ, e.g., by at least one amino acid residue, from a naturally occurring Cas9 molecule.

The terms “gRNA” and “guide RNA” are used interchangeably throughout and refer to a nucleic acid that promotes the specific targeting or homing of a gRNA/Cas9 molecule complex to a target nucleic acid. A gRNA can be unimolecular (having a single RNA molecule), sometimes referred to herein as sgRNAs, or modular (comprising more than one, and typically two, separate RNA molecules). A gRNA may bind to a target domain in the genome of a host cell. The gRNA may comprise a targeting domain that may be partially or completely complementary to the target domain. The gRNA may also comprise a “scaffold sequence,” (e.g., a tracrRNA sequence), that recruits a Cas9 molecule to a target domain bound to a gRNA sequence (e.g., by the targeting domain of the gRNA sequence). The scaffold sequence may comprise at least one stem loop structure and recruits an endonuclease. Exemplary scaffold sequences can be found, for example, in Jinek, et al. Science (2012) 337(6096):816-821, Ran, et al. Nature Protocols (2013) 8:2281-2308, PCT Publication No. WO2014/093694, and PCT Publication No. WO2013/176772.

The term “mutation” is used herein to refer to a genetic change (e.g., insertion, deletion, inversion, or substitution) in a nucleic acid compared to a reference sequence, e.g., the corresponding sequence of a cell not having such a mutation or corresponding wild-type nucleic acid sequence. In some embodiments provided herein, a mutation in a gene encoding a lineage-specific cell-surface antigen (e.g., CD33 (Siglec-3), CLL-1, CD123, CD327 (Siglec-6), and/or CD312 (EMR2)) results in a loss of expression of the lineage-specific cell-surface antigen in a cell harboring the mutation. In some embodiments, a mutation to a gene detargetizes the protein produced by the gene. In some embodiments, a detargetized lineage-specific cell-surface antigen protein is not bound by, or is bound at a lower level by, an agent that targets the lineage-specific cell-surface antigen. In some embodiments, a mutation in a gene encoding a lineage-specific cell-surface antigen (e.g., CD33 (Siglec-3), CLL-1, CD123, CD327 (Siglec-6), and/or CD312 (EMR2)) results in the expression of a variant form of the lineage-specific cell-surface antigen that is not bound by an immunotherapeutic agent targeting the lineage-specific cell-surface antigen, or bound at a significantly lower level than the non-mutated lineage-specific cell-surface antigen form encoded by the gene. In some embodiments, a cell harboring a genomic mutation in the lineage-specific cell-surface antigen (e.g., CD33 (Siglec-3), CLL-1, CD123, CD327 (Siglec-6), and/or CD312 (EMR2)) gene as provided herein is not bound by, or is bound at a significantly lower level by an immunotherapeutic agent that targets the lineage-specific cell-surface antigen, e.g., an anti-CD33 antibody or chimeric antigen receptor (CAR), an anti-CLL-1 antibody or chimeric antigen receptor (CAR), an anti-CD123 antibody or chimeric antigen receptor (CAR), an anti-CD19 antibody or chimeric antigen receptor (CAR), an anti-CD30 antibody or chimeric antigen receptor (CAR), an anti-CD5 antibody or chimeric antigen receptor (CAR), an anti-CD6 antibody or chimeric antigen receptor (CAR), an anti-CD7 antibody or chimeric antigen receptor (CAR), an anti-CD34 antibody or chimeric antigen receptor (CAR), an anti-CD38 antibody or chimeric antigen receptor (CAR), and/or an anti-BCMA antibody or chimeric antigen receptor (CAR). In some embodiments, a cell harboring a genomic mutation in the lineage-specific cell-surface antigen (e.g., CD33 (Siglec-3), CLL-1, CD123, CD327 (Siglec-6), and/or CD312 (EMR2)) gene as provided herein is not bound by, or is bound at a significantly lower level by an immunotherapeutic agent that targets the lineage-specific cell-surface antigen, e.g., an antibody or a chimeric antigen receptor (CAR) that targets CD33 (Siglec-3), CLL-1, CD123, CD327 (Siglec-6), and/or CD312 (EMR2).

In some embodiments, a cell harboring a genomic mutation in a cell-surface antigen gene as provided herein is not bound by, or is bound at a significantly lower level by an immunotherapeutic agent that targets the cell-surface antigen, e.g., an antibody or chimeric antigen receptor (CAR). In some embodiments, the immunotherapeutic agent, e.g., an antibody or chimeric antigen receptor (CAR), targets a cell-surface antigen is CD1a, CD1b, CD1c, CD1d, CD1e, CD2, CD3d, CD3e, CD3g, CD4, CD5, CD6, CD7, CD8a, CD8b, CD9, CD10, CD11a, CD11b, CD11c, CD11d, CD13, CD14, CD15, CD16a, CD16b, CD17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c,CD61, CD62E, CD62L, CD62P, CD63, CD64, CD65s, CD66a, CD66b, CD66c, CD66d, CD66e, CD66f, CD67, CD68, CD69, CD70, CD71, CD72, CD73, CD74, CD75, CD75s,CD77, CD79a, CD79b, CD80, CD81, CD82, CD83, CD84, CD85a, CD85b, CD85c, CD85d, CD85e, CD85f, CD85g, CD85h, CD85i, CD85j, CD85k, CD86, CD87, CD88, CD89, CD90, CD91, CD92, CD93, CD94, CD95, CD96, CD97, CD98, CD99, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD122, CD123, CD124, CD125, CD126, CD127, CD128a, CD128b, CD129, CD130, CD131, CD132, CD133,CD134, CD135, CD136, CD137, CD138, CD139,CD140a, CD140b, CD141, CD142, CD143, CD144, CD146, CD147, CD148, CD150, CD151, CD152, CD153, CD154, CD155, CD156a, CD156b, CD156c, CD157, CD158a, CD158b1, CD158b2, CD158c,CD158d, CD158e1, CD158e2, CD158f, CD158g,CD158h, CD158i, CD158j, CD158k, CD159a, CD159c, CD160, CD161, CD162, CD163, CD164, CD165,CD166, CD167a, CD167b, CD168, CD169, CD170, CD171, CD172a, CD172b, CD172g, CD173, CD174, CD175,CD175s, CD176, CD177, CD178, CD179a, CD179b, CD180, CD181, CD182, CD183, CD184, CD185, CD186, CD191, CD192, CD193, CD194, CD195, CD196, CD197, CD198, CD199, CD200, CD201, CD202b, CD203c, CD204, CD205, CD206, CD207, CD208, CD209, CD210a, CD210b, CD212, CD213al, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD238, CD239, CD240CE, CD240D, CD241, CD242, CD243, CD244, CD245,CD246, CD247, CD248, CD249, CD252, CD253, CD254, CD256, CD257, CD258, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300d, CD300e, CD300f, CD300g, CD301, CD302, CD303,CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD360, CD361, CD362, CD363, CD364, CD365, CD366, CD367, CD368, CD369, CD370, CD371, or any combination thereof. See also examples of lineage-specific cell-surface antigens from BD Biosciences Human CD Marker Chart, https://www.bdbiosciences.com/content/dam/bdb/campaigns/reagent-education/BD_Reagents_CDMarkerHuman_Poster.pdf (incorporated by reference in it's entirety).

The “targeting domain” of the gRNA is complementary to the “target domain” on the target nucleic acid. The strand of the target nucleic acid comprising the nucleotide sequence complementary to the core domain of the gRNA is referred to herein as the “complementary strand” of the target nucleic acid. The targeting domain mediates targeting of the gRNA-bound RNA-guided nuclease to a target site. Guidance on the selection of targeting domains can be found, e.g., in Fu Y et al, Nat Biotechnol 2014 (doi: 10.1038/nbt.2808) and Sternberg SH et al., Nature 2014 (doi: 10.1038/naturel3011).

The term “base editing” refers to a genome editing technology which includes the use of a base editor, e.g., a nuclease-impaired or partially nuclease impaired gene editing enzyme (e.g., RNA-guided CRISPR/Cas protein) fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide. See, e.g., Komor et al. Nature (2016) 533: 420-424; Rees et al. Nat. Rev. Genet. (2018) 19(12): 770-788; Anzalone et al. Nat. Biotechnol. (2020) 38: 824-844.

The term “target domain”, “target site”, or “target sequence” refers to a sequence within a nucleic acid molecule (e.g., a DNA molecule) that is deaminated by a base editor as described herein. In some embodiments, the target sequence is a polynucleotide (e.g., a double-stranded DNA molecule), wherein the polynucleotide comprises a coding strand and a complementary strand. The meaning of a “coding strand” and “complementary strand” is the common meaning of the terms in the art. In some embodiments, the target sequence is a sequence in the genome of a mammal. In some embodiments, the target sequence is a sequence in the genome of a human. The term “target codon” refers to the amino acid codon that is edited by the base editor and converted to a different codon via deamination of a nucleobase. In some embodiments, the target codon is edited in the coding strand. In some embodiments, the target codon is edited in the complementary strand.

The terms “surface antigen” or “cell-surface antigen” refers to an antigen on the surface of a cell that is extracellularly accessible during at least one cell cycle or developmental stage of the cell, including antigens that are extracellularly accessible during all stages of the cell cycle. “Extracellularly accessible” in this context refers to an antigen that can be bound by an agent, such as an antibody, provided outside the cell without need for permeabilization of the cell membrane. As used herein, the term “cell-surface antigen”can comprise a protein, a peptide, a sugar, a lipid, or other moiety that is presented on the surface of a cell, such as on the surface of hematopoietic stem and progenitor cells (HSPCs).

The term “antigen” refers to the portion of a macromolecule (e.g., a polypeptide) which is specifically recognized by a component of the immune system, e.g., an antibody or antigen-binding portion thereof. As used herein, the term “antigen” encompasses any molecule that provokes an immune response. This immune response may involve either antibody production, or the activation of specific immunologically-competent cells, or both. A skilled artisan will understand that any nucleic acid, which comprises a nucleotide sequences encoding a protein or portion thereof that elicits an immune response therefore encodes an “antigen” as that term is used herein. Cell-surface antigens include, but are not limited to, a cell surface molecule such as a protein, a peptide, a sugar, a lipid, or other moiety on the cell surface.

Exemplary cell-surface antigens include, but are not limited to, CD1a, CD1b, CD1c, CD1d, CD1e, CD2, CD3d, CD3e, CD3g, CD4, CD5, CD6, CD7, CD8a, CD8b, CD9, CD10, CD11a, CD11b, CD11c, CD11d, CD13, CD14, CD15, CD16a, CD16b, CD17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c,CD61, CD62E, CD62L, CD62P, CD63, CD64, CD65s, CD66a, CD66b, CD66c, CD66d, CD66e, CD66f, CD67, CD68, CD69, CD70, CD71, CD72, CD73, CD74, CD75, CD75s,CD77, CD79a, CD79b, CD80, CD81, CD82, CD83, CD84, CD85a, CD85b, CD85c, CD85d, CD85e, CD85f, CD85g, CD85h, CD85i, CD85j, CD85k, CD86, CD87, CD88, CD89, CD90, CD91, CD92, CD93, CD94, CD95, CD96, CD97, CD98, CD99, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD122, CD123, CD124, CD125, CD126, CD127, CD128a, CD128b, CD129, CD130, CD131, CD132, CD133,CD134, CD135, CD136, CD137, CD138, CD139,CD140a, CD140b, CD141, CD142, CD143, CD144, CD146, CD147, CD148, CD150, CD151, CD152, CD153, CD154, CD155, CD156a, CD156b, CD156c, CD157, CD158a, CD158b1, CD158b2, CD158c,CD158d, CD158e1, CD158e2, CD158f, CD158g,CD158h, CD158i, CD158j, CD158k, CD159a, CD159c, CD160, CD161, CD162, CD163, CD164, CD165,CD166, CD167a, CD167b, CD168, CD169, CD170, CD171, CD172a, CD172b, CD172g, CD173, CD174, CD175,CD175s, CD176, CD177, CD178, CD179a, CD179b, CD180, CD181, CD182, CD183, CD184, CD185, CD186, CD191, CD192, CD193, CD194, CD195, CD196, CD197, CD198, CD199, CD200, CD201, CD202b, CD203c, CD204, CD205, CD206, CD207, CD208, CD209, CD210a, CD210b, CD212, CD213al, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD238, CD239, CD240CE, CD240D, CD241, CD242, CD243, CD244, CD245,CD246, CD247, CD248, CD249, CD252, CD253, CD254, CD256, CD257, CD258, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300d, CD300e, CD300f, CD300g, CD301, CD302, CD303,CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD360, CD361, CD362, CD363, CD364, CD365, CD366, CD367, CD368, CD369, CD370, CD371, or any combination thereof. See also examples of lineage-specific cell-surface antigens from BD Biosciences Human CD Marker Chart, https://www.bdbiosciences.com/content/dam/bdb/campaigns/reagent-education/BD_Reagents_CDMarkerHuman_Poster.pdf.

The term “exon” refers to a nucleic acid sequence that comprises the coding sequence of a gene. A gene typically includes more than one exon, which are separated by an intron in between.

The term “intron” refers to a nucleic acid sequence flanking the coding sequences of a gene. The term “introns” encompasses noncoding sequences located inside precursor mRNA (pre-mRNA) transcripts that are typically excised before nuclear export. Splicing of pre-mRNA requires sequence motifs in the intron and is mediated by a ribonucleoprotein complex called the spliceosome. Introns typically contain 5′ donor and 3′ acceptor splice sites, usually with GU and AG dinucleotides at the respective intron ends and a branch point located within the intron. At the 5′ end the DNA nucleotides can be GT (GU in the pre-mRNA), and at the 3′ end they can be “AG”. These nucleotides are part of the splicing sites. In some embodiments, the intron is spliced out of or removed from an RNA or mRNA sequence in which it is present. During splicing, the branch point nucleotide initiates a nucleophilic attack on the 5′ donor splice site. The free end of the upstream intron then initiates a second nucleophilic attack on the 3′ acceptor splice site, releasing the intron as an RNA lariat and covalently combining the two exons. Introns are typically removed by the major spliceosome, a large ribonucleoprotein (RNP) complex found primarily within the nucleus of eukaryotic cells. The spliceosome is assembled from small nuclear RNAs (snRNA) and numerous proteins. Base pairing of the snRNAs to the intron and to each other, plus protein-protein and protein-RNA interactions of splicing factors, position the splice sites for splicing.

The term “splice donor site” refers to a nucleic acid sequence or domain on the 5′ end of an intron. The splice donor site, in one embodiment, marks the start of the intron and/or the intron's boundary with an immediately preceding coding sequence (e.g., an exon).

The term “splice acceptor site” refers to a nucleic acid sequence or domain on the 3′ end of an intron. In some embodiments, the splice acceptor site marks the start of the intron and its boundary with the following coding sequence (e.g., an exon). In some embodiment, the splice acceptor site comprises an intron branch point. In some embodiments, the intron branch point is the point to which the 5′ end of the intron becomes joined during the process of splicing. In some embodiments, the splice acceptor sequence and the intron branch site are adjacent to each other. In some embodiments, the splice acceptor sequence and the intron branch site may be separated, e.g., the branch site may be further 5′ of the splice acceptor sequence.

The term “splicing branch point” refers to the nucleotide of an intron that participates in splicing by promoting the formation of a branched RNA lariat.

The term“splice site” refers to a sequence or domain of a nucleic acid present at either the 5′ end or the 3′ end of an intron as described herein.

The term “splice site mutation” is a genetic mutation that inserts, deletes, or changes one or more nucleotides in the specific site at which splicing takes place during the processing of precursor messenger RNA into mature messenger RNA. The splicing process itself is controlled, at least in part, by the splice donor and splice acceptor sequences which surround each exon. Mutations in these sequences may lead to, for example, retention of large segments of intronic DNA by the mRNA, or to entire exons being spliced out of the mRNA. Such changes can potentially result in production of a nonfunctional protein.

### Nucleases/Gene Editing Enzymes

In some embodiments, a cell (e.g., HSC or HPC) described herein is made using a nuclease described herein. Exemplary nucleases include CRISPR/Cas molecules (also referred to as CRISPR/Cas nucleases, Cas nuclease, e.g., Cas9), TALENs, ZFNs, and meganucleases. In some embodiments, a nuclease is used in combination with a lineage-specific cell-surface antigen (e.g., CD33 (Siglec-3), CLL-1, CD123, CD327 (Siglec-6), and/or CD312 (EMR2)) gRNA described herein (e.g., according to Tables 1-19). Some aspects of this disclosure provide compositions and methods for generating the genetically engineered cells described herein, e.g., genetically engineered cells comprising a modification in their genome that results in a loss of expression of a lineage-specific cell-surface antigen (e.g., CD33 (Siglec-3), CLL-1, CD123, CD327 (Siglec-6), and/or CD312 (EMR2)), or expression of a variant form of the lineage-specific cell-surface antigen that is not recognized by an immunotherapeutic agent targeting the lineage-specific cell-surface antigen. Such compositions and methods provided herein include, without limitation, suitable strategies and approaches for genetically engineering cells, e.g., by using nucleases, such as CRISPR/Cas nucleases, and suitable RNAs able to bind such nucleases and target them to a suitable target site within the genome of a cell to effect a genomic modification resulting in a loss of expression of the lineage-specific cell-surface antigen, or expression of a variant form of the lineage-specific cell-surface antigen that is not recognized by an immunotherapeutic agent targeting the lineage-specific cell-surface antigen (e.g., CD33 (Siglec-3), CLL-1, CD123, CD327 (Siglec-6), and/or CD312 (EMR2)).

In some embodiments, a genetically engineered cell (e.g., a genetically engineered hematopoietic cell, such as, for example, a genetically engineered hematopoietic stem or progenitor cell or a genetically engineered immune effector cell) described herein is generated via genome editing technology, which includes any technology capable of introducing targeted changes, also referred to as “edits,” into the genome of a cell using a nuclease, such as any of the nucleases described herein.

One exemplary suitable genome editing technology is “gene editing,” comprising the use of a nuclease, e.g., an RNA- RNA-guided nuclease, such as a CRISPR/Cas nuclease, to introduce targeted single- or double-stranded DNA breaks in the genome of a cell, which trigger cellular repair mechanisms, such as, for example, nonhomologous end joining (NHEJ), microhomology-mediated end joining (MMEJ, also sometimes referred to as “alternative NHEJ” or “alt-NHEJ”), or homology-directed repair (HDR) that typically result in an altered nucleic acid sequence (e.g., via nucleotide or nucleotide sequence insertion, deletion, inversion, or substitution) at or immediately proximal to the site of the nuclease cut. See, Yeh et al. Nat. Cell. Biol. (2019) 21: 1468-1478; e.g., Hsu et al. Cell (2014) 157: 1262-1278; Jasin et al. DNA Repair (2016) 44: 6-16; Sfeir et al. Trends Biochem. Sci. (2015) 40: 701-714.

Another exemplary suitable genome editing technology is “base editing,” which includes the use of a base editor, e.g., a nuclease-impaired or partially nuclease impaired enzyme (e.g., RNA-guided CRISPR/Cas protein) fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide. See, e.g., Komor et al. Nature (2016) 533: 420-424; Rees et al. Nat. Rev. Genet. (2018) 19(12): 770-788; Anzalone et al. Nat. Biotechnol. (2020) 38: 824-844.

Yet another exemplary suitable genome editing technology includes “prime editing,” which includes the introduction of new genetic information, e.g., an altered nucleotide sequence, into a specifically targeted genomic site using a catalytically impaired or partially catalytically impaired nuclease (e.g., RNA-guided nuclease, e.g., a CRISPR/Cas nuclease), fused to an engineered reverse transcriptase (RT) domain. The Cas/RT fusion is targeted to a target site within the genome by a guide RNA that also comprises a nucleic acid sequence encoding the desired edit, and that can serve as a primer for the RT. See, e.g., Anzalone et al. Nature (2019) 576 (7785): 149-157.

### Cas9 Molecules

In some embodiments, use of genome editing technology features the use of a suitable RNA-guided nuclease, which, in some embodiments, e.g., for base editing or prime editing, may be catalytically impaired, or partially catalytically impaired. Examples of suitable RNA-guided nucleases include CRISPR/Cas nucleases, such as Cas9 or other Cas nuclease, such as Cas12a/Cpf1.

In some embodiments, a lineage-specific cell-surface antigen (e.g., CD33 (Siglec-3), CLL-1, CD123, CD327 (Siglec-6), and/or CD312 (EMR2)) gRNA described herein is complexed with a Cas9 molecule. Various Cas9 molecules can be used. In some embodiments, a Cas9 molecule is selected that has the desired PAM specificity to target the gRNA/Cas9 molecule complex to the target domain in the lineage-specific cell-surface antigen (e.g., CD33 (Siglec-3), CLL-1, CD123, CD327 (Siglec-6), and/or CD312 (EMR2)). In some embodiments, genetically engineering a cell also comprises introducing one or more (e.g., 1, 2, 3 or more) Cas9 molecules into the cell.

In some embodiments, a CD33 gRNA described herein is complexed with a Cas9 molecule. Various Cas9 molecules can be used. In some embodiments, a Cas9 molecule is selected that has the desired PAM specificity to target the gRNA/Cas9 molecule complex to the target domain in CD33. In some embodiments, genetically engineering a cell also comprises introducing one or more (e.g., 1, 2, 3 or more) Cas9 molecules into the cell.

In some embodiments, a CLL-1 gRNA described herein is complexed with a Cas9 molecule. Various Cas9 molecules can be used. In some embodiments, a Cas9 molecule is selected that has the desired PAM specificity to target the gRNA/Cas9 molecule complex to the target domain in CLL-1. In some embodiments, genetically engineering a cell also comprises introducing one or more (e.g., 1, 2, 3 or more) Cas9 molecules into the cell.

In some embodiments, a CD123 gRNA described herein is complexed with a Cas9 molecule. Various Cas9 molecules can be used. In some embodiments, a Cas9 molecule is selected that has the desired PAM specificity to target the gRNA/Cas9 molecule complex to the target domain in CD123. In some embodiments, genetically engineering a cell also comprises introducing one or more (e.g., 1, 2, 3 or more) Cas9 molecules into the cell.

In some embodiments, a CD327 (Siglec-6) gRNA described herein is complexed with a Cas9 molecule. Various Cas9 molecules can be used. In some embodiments, a Cas9 molecule is selected that has the desired PAM specificity to target the gRNA/Cas9 molecule complex to the target domain in CD327 (Siglec-6). In some embodiments, genetically engineering a cell also comprises introducing one or more (e.g., 1, 2, 3 or more) Cas9 molecules into the cell.

In some embodiments, a CD312 (EMR2) gRNA described herein is complexed with a Cas9 molecule. Various Cas9 molecules can be used. In some embodiments, a Cas9 molecule is selected that has the desired PAM specificity to target the gRNA/Cas9 molecule complex to the target domain in CD312 (EMR2). In some embodiments, genetically engineering a cell also comprises introducing one or more (e.g., 1, 2, 3 or more) Cas9 molecules into the cell.

Cas9 molecules of a variety of species can be used in the methods and compositions described herein. In embodiments, the Cas9 molecule is of, or derived from, Streptococcus pyogenes (SpCas9), Staphylococcus aureus (SaCas9), or Streptococcus thermophilus (StCas9). Additional suitable Cas9 molecules include those of, or derived from, Staphylococcus aureus, Neisseria meningitidis (NmCas9), Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni (CjCas9), Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In some embodiments, catalytically impaired, or partially impaired, variants of such Cas9 nucleases may be used. Additional suitable Cas9 nucleases, and nuclease variants, will be apparent to those of skill in the art based on the present disclosure. The disclosure is not limited in this respect.

In some embodiments, the Cas9 molecule is a naturally occurring Cas9 molecule. In some embodiments, the Cas9 molecule is an engineered, altered, or modified Cas9 molecule that differs, e.g., by at least one amino acid residue, from a reference sequence, e.g., the most similar naturally occurring Cas9 molecule or a sequence of Table 50 of PCT Publication No. WO 2015/157070, which is herein incorporated by reference in its entirety. In some embodiments, the Cas9 molecule comprises Cpf1 or a fragment or variant thereof.

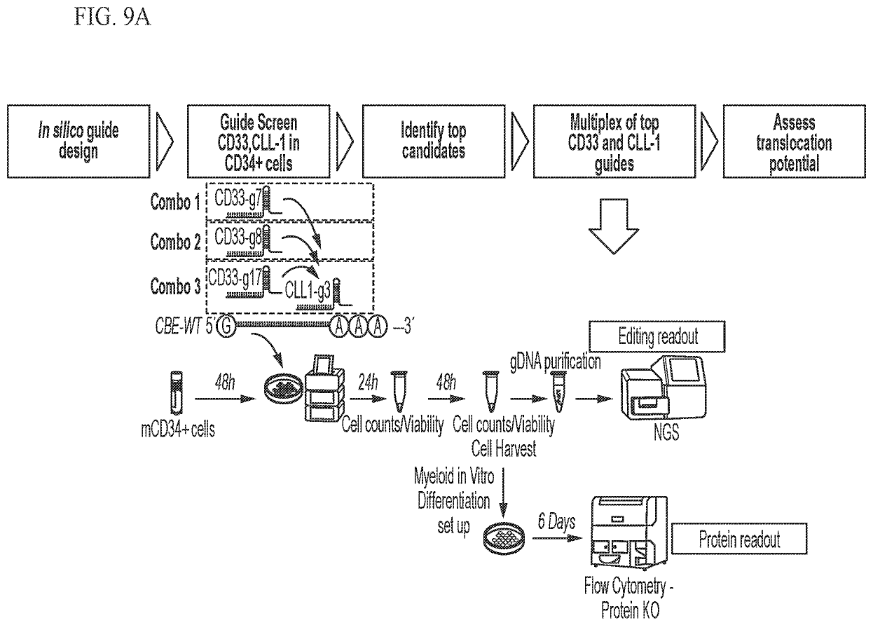

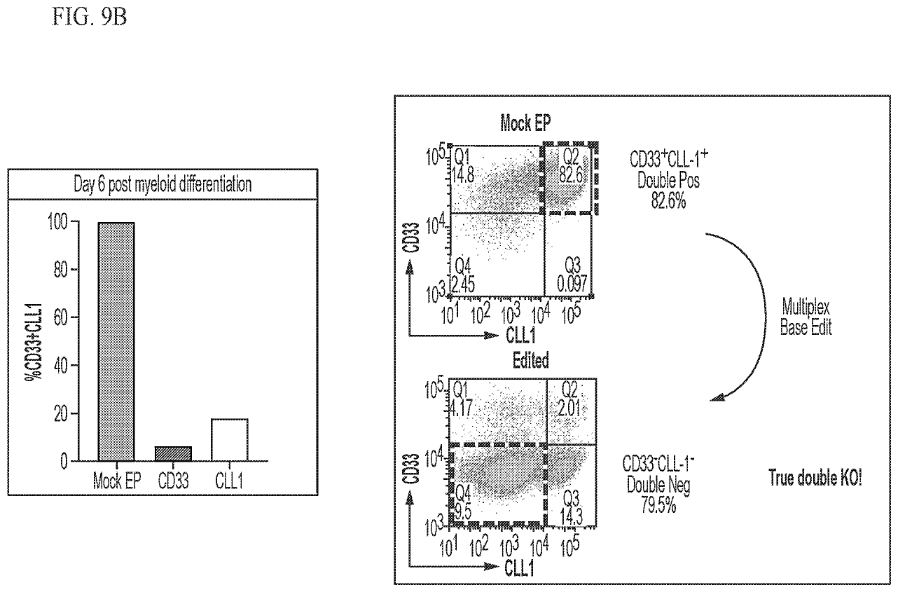

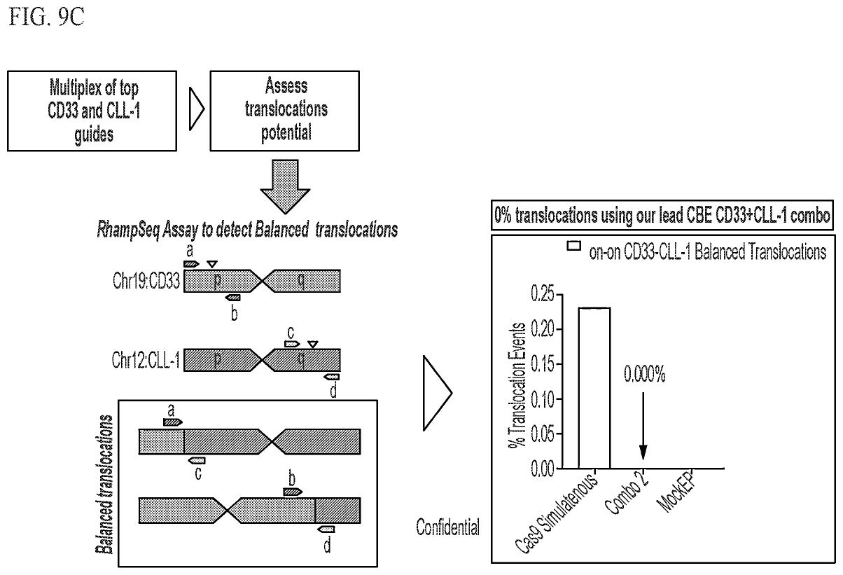

A naturally occurring Cas9 molecule typically comprises two lobes: a recognition (REC) lobe and a nuclease (NUC) lobe; each of which further comprises domains described, e.g., in PCT Publication No. WO 2015/157070, e.g., in FIGS. 9A-9B therein (which application is incorporated herein by reference in its entirety).

The REC lobe comprises the arginine-rich bridge helix (BH), the REC1 domain, and the REC2 domain. The REC lobe appears to be a Cas9-specific functional domain. The BH domain is a long alpha helix and arginine rich region and comprises amino acids 60-93 of the sequence of S. pyogenes Cas9. The REC1 domain is involved in recognition of the repeat:anti-repeat duplex, e.g., of a gRNA or a tracrRNA. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat: anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule, and comprises amino acids 1099-1368 of the sequence of S. pyogenes Cas9.

Crystal structures have been determined for naturally occurring bacterial Cas9 molecules (Jinek et al., Science, 343(6176): 1247997, 2014) and for S. pyogenes Cas9 with a guide RNA (e.g., a synthetic fusion of crRNA and tracrRNA) (Nishimasu et al., Cell, 156:935-949, 2014; and Anders et al., Nature, 2014, doi: 10.1038/naturel3579).

In some embodiments, a Cas9 molecule described herein has nuclease activity, e.g., double strand break activity in or directly proximal to a target site. In some embodiments, the Cas9 molecule has been modified to inactivate one of the catalytic residues of the endonuclease. In some embodiments, the Cas9 molecule is a nickase and produces a single stranded break. See, e.g., Dabrowska et al. Frontiers in Neuroscience (2018) 12(75). It has been shown that one or more mutations in the RuvC and HNH catalytic domains of the enzyme may improve Cas9 efficiency. See, e.g., Sarai et al. Currently Pharma. Biotechnol. (2017) 18(13). In some embodiments, the Cas9 molecule is fused to a second domain, e.g., a domain that modifies DNA or chromatin, e.g., a deaminase or demethylase domain. In some such embodiments, the Cas9 molecule is modified to eliminate its endonuclease activity.

In some embodiments, a Cas nuclease (e.g., a Cas9 molecule or a Cas/gRNA complex) described herein is administered together with a template for homology directed repair (HDR). In some embodiments, a Cas9 molecule described herein is administered without a HDR template.

In some embodiments, the Cas9 molecule is modified to enhance specificity of the enzyme (e.g., reduce off-target effects, maintain robust on-target cleavage). In some embodiments, the Cas9 molecule is an enhanced specificity Cas9 variant (e.g., eSPCas9). See, e.g., Slaymaker et al. Science (2016) 351 (6268): 84-88. In some embodiments, the Cas9 molecule is a high fidelity Cas9 variant (e.g., SpCas9-HF1). See, e.g., Kleinstiver et al. Nature (2016) 529: 490-495.

Various Cas9 molecules are known in the art and may be obtained from various sources and/or engineered/modified to modulate one or more activities or specificities of the enzymes. In some embodiments, the Cas9 molecule has been engineered/modified to recognize one or more PAM sequence. In some embodiments, the Cas9 molecule has been engineered/modified to recognize one or more PAM sequence that is different than the PAM sequence the Cas9 molecule recognizes without engineering/modification. In some embodiments, the Cas9 molecule has been engineered/modified to reduce off-target activity of the enzyme.

In some embodiments, the nucleotide sequence encoding the Cas9 molecule is modified further to alter the specificity of the endonuclease activity (e.g., reduce off-target cleavage, decrease the endonuclease activity or lifetime in cells, increase homology-directed recombination and reduce non-homologous end joining). See, e.g., Komor et al. Cell (2017) 168: 20-36. In some embodiments, the nucleotide sequence encoding the Cas9 molecule is modified to alter the PAM recognition of the endonuclease. For example, the Cas9 molecule SpCas9 recognizes PAM sequence NGG, whereas relaxed variants of the SpCas9 comprising one or more modifications of the endonuclease (e.g., VQR SpCas9, EQR SpCas9, VRER SpCas9) may recognize the PAM sequences NGA, NGAG, NGCG. PAM recognition of a modified Cas9 molecule is considered “relaxed” if the Cas9 molecule recognizes more potential PAM sequences as compared to the Cas9 molecule that has not been modified. For example, the Cas9 molecule SaCas9 recognizes PAM sequence NNGRRT, whereas a relaxed variant of the SaCas9 comprising one or more modifications (e.g., KKH SaCas9) may recognize the PAM sequence NNNRRT. In one example, the Cas9 molecule FnCas9 recognizes PAM sequence NNG, whereas a relaxed variant of the FnCas9 comprising one or more modifications of the endonuclease (e.g., RHA FnCas9) may recognize the PAM sequence YG. In one example, the Cas9 molecule is a Cpf1 endonuclease comprising substitution mutations S542R and K607R and recognize the PAM sequence TYCV. In one example, the Cas9 molecule is a Cpf1 endonuclease comprising substitution mutations S542R, K607R, and N552R and recognize the PAM sequence TATV. See, e.g., Gao et al. Nat. Biotechnol. (2017) 35(8): 789-792.

In some embodiments, more than one (e.g., 2, 3, or more) Cas9 molecules are used. In some embodiments, at least one of the Cas9 molecule is a Cas9 enzyme. In some embodiments, at least one of the Cas molecules is a Cpf1 enzyme. In some embodiments, at least one of the Cas9 molecule is derived from Streptococcus pyogenes. In some embodiments, at least one of the Cas9 molecule is derived from Streptococcus pyogenes and at least one Cas9 molecule is derived from an organism that is not Streptococcus pyogenes.

In some embodiments, the Cas9 molecule is a base editor. In some embodiments, a base editor is used to a create a genomic modification resulting in a loss of expression of a lineage-specific cell-surface antigen (e.g., CD33 (Siglec-3), CLL-1, CD123, CD327 (Siglec-6), and/or CD312 (EMR2)), or in expression of a lineage-specific cell-surface antigen variant not targeted by an immunotherapy. Base editor endonuclease generally comprises a catalytically inactive Cas9 molecule fused to a functional domain, e.g., a deaminase domain. See, e.g., Eid et al. Biochem. J. (2018) 475(11): 1955-1964; Rees et al. Nature Reviews Genetics (2018) 19:770-788. In some embodiments, the catalytically inactive Cas9 molecule is referred to as “dead Cas” or “dCas9.” In some embodiments, the catalytically inactive Cas molecule has reduced activity and is, e.g., a nickase (referred to as “nCas”). In some embodiments, the endonuclease comprises a dCas9 fused to one or more uracil glycosylase inhibitor (UGI) domains. In some embodiments, the endonuclease comprises a dCas9 fused to an adenine base editor (ABE), for example an ABE evolved from the RNA adenine deaminase TadA. In some embodiments, the endonuclease comprises a dCas9 fused to cytidine deaminase enzyme (e.g., APOBEC deaminase, pmCDA1, activation-induced cytidine deaminase (AID)). In some embodiments, the catalytically inactive Cas9 molecule has reduced activity and is nCas9. In some embodiments, the catalytically inactive Cas9 molecule (dCas9) is fused to one or more uracil glycosylase inhibitor (UGI) domains. In some embodiments, the Cas9 molecule comprises an inactive Cas9 molecule (dCas9) fused to an adenine base editor (ABE), for example an ABE evolved from the RNA adenine deaminase TadA. In some embodiments, the Cas9 molecule comprises a nCas9 fused to an adenine base editor (ABE), for example an ABE evolved from the RNA adenine deaminase TadA. In some embodiments, the Cas9 molecule comprises a dCas9 fused to cytidine deaminase enzyme (e.g., APOBEC deaminase, pmCDA1, activation-induced cytidine deaminase (AID)). In some embodiments, the Cas9 molecule comprises a nCas9 fused to cytidine deaminase enzyme (e.g., APOBEC deaminase, pmCDA1, activation-induced cytidine deaminase (AID)).

Examples of base editors include, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, ×BE3, eA3A-BE3, BE-PLUS, TAM, CRISPR-X, ABE7.9, ABE7.10, ABE7.10*, xABE, ABESa, VQR-ABE, VRER-ABE, Sa(KKH)-ABE, and CRISPR-SKIP. Additional examples of base editors can be found, for example, in US Publication No. 2018/0312825A1, US Publication No. 2018/0312828A1, and PCT Publication No. WO 2018/165629A1, which are incorporated by reference herein in their entireties.

In some embodiments, the base editor has been further modified to inhibit base excision repair at the target site and induce cellular mismatch repair. Any of the Cas9 molecules described herein may be fused to a Gam domain (bacteriophage Mu protein) to protect the Cas9 molecule from degradation and exonuclease activity. See, e.g., Eid et al. Biochem. J. (2018) 475(11): 1955-1964.

In some embodiments, the Cas9 molecule belongs to class 2 type V of Cas endonuclease. Class 2 type V Cas endonucleases can be further categorized as type V-A, type V-B, type V-C, and type V-U. See, e.g., Stella et al. Nature Structural & Molecular Biology (2017) 24: 882-892. In some embodiments, the Cas molecule is a type V-A Cas endonuclease, such as a Cpf1 (Cas12a) nuclease. In some embodiments, the Cas9 molecule is a type V-B Cas endonuclease, such as a C2c1 endonuclease. See, e.g., Shmakov et al. Mol Cell (2015) 60: 385-397. In some embodiments, the Cas molecule is MAD7™. Alternatively or in addition, the Cas9 molecule is a Cpf1 nuclease or a variant thereof. As will be appreciated by one of skill in the art, the Cpf1 nuclease may also be referred to as Cas12a. See, e.g., Strohkendl et al. Mol. Cell (2018) 71: 1-9. In some embodiments, a composition or method described herein involves, or a host cell expresses a Cpf1 nuclease derived from Provetella spp. or Francisella spp., Acidaminococcus sp. (AsCpf1), Lachnospiraceae bacterium (LpCpf1), or Eubacterium rectale. In some embodiments, the nucleotide sequence encoding the Cpf1 nuclease may be codon optimized for expression in a host cell. In some embodiments, the nucleotide sequence encoding the Cpf1 endonuclease is further modified to alter the activity of the protein.

Both naturally occurring and modified variants of CRISPR/Cas nucleases are suitable for use according to aspects of this disclosure. For example, dCas or nickase variants, Cas variants having altered PAM specificities, and Cas variants having improved nuclease activities are embraced by some embodiments of this disclosure. In some embodiments, catalytically inactive variants of Cas molecules (e.g., of Cas9 or Cas12a) are used according to the methods described herein. A catalytically inactive variant of Cpf1 (Cas12a) may be referred to dCas12a. As described herein, catalytically inactive variants of Cpf1 maybe fused to a function domain to form a base editor. See, e.g., Rees et al. Nature Reviews Genetics (2018) 19:770-788. In some embodiments, the catalytically inactive Cas9 molecule is dCas9. In some embodiments, the endonuclease comprises a dCas12a fused to one or more uracil glycosylase inhibitor (UGI) domains. In some embodiments, the Cas9 molecule comprises a dCas12a fused to an adenine base editor (ABE), for example an ABE evolved from the RNA adenine deaminase TadA. In some embodiments, the Cas molecule comprises a dCas12a fused to cytidine deaminase enzyme (e.g., APOBEC deaminase, pmCDA1, activation-induced cytidine deaminase (AID)).

Alternatively or in addition, the Cas9 molecule may be a Cas14 endonuclease or variant thereof. Cas14 endonucleases are derived from archaea and tend to be smaller in size (e.g., 400-700 amino acids). Additionally Cas14 endonucleases do not require a PAM sequence. See, e.g., Harrington et al. Science (2018).

Any of the Cas9 molecules described herein may be modulated to regulate levels of expression and/or activity of the Cas9 molecule at a desired time. For example, it may be advantageous to increase levels of expression and/or activity of the Cas9 molecule during particular phase(s) of the cell cycle. It has been demonstrated that levels of homology-directed repair are reduced during the G1 phase of the cell cycle, therefore increasing levels of expression and/or activity of the Cas9 molecule during the S phase, G2 phase, and/or M phase may increase homology-directed repair following the Cas endonuclease editing. In some embodiments, levels of expression and/or activity of the Cas9 molecule are increased during the S phase, G2 phase, and/or M phase of the cell cycle. In one example, the Cas9 molecule fused to the N-terminal region of human Geminin. See, e.g., Gutschner et al. Cell Rep. (2016) 14(6): 1555-1566. In some embodiments, levels of expression and/or activity of the Cas9 molecule are reduced during the G1 phase. In one example, the Cas9 molecule is modified such that it has reduced activity during the G1 phase. See, e.g., Lomova et al. Stem Cells (2018).

Alternatively or in addition, any of the Cas9 molecules described herein may be fused to an epigenetic modifier (e.g., a chromatin-modifying enzyme, e.g., DNA methylase, histone deacetylase). See, e.g., Kungulovski et al. Trends Genet. (2016) 32(2):101-113. Cas9 molecule fused to an epigenetic modifier may be referred to as “epieffectors” and may allow for temporal and/or transient endonuclease activity. In some embodiments, the Cas9 molecule is a dCas9 fused to a chromatin-modifying enzyme.

### Base Editors

In some embodiments, a cell or cell population described herein is produced using base editing technology. As described above, base editing includes the use of a base editor, e.g., a nuclease-impaired or partially nuclease impaired enzyme (e.g., RNA-guided CRISPR/Cas protein) fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide. See, e.g., Komor et al. Nature (2016) 533: 420-424; Rees et al. Nat. Rev. Genet. (2018) 19(12): 770-788; Anzalone et al. Nat. Biotechnol. (2020) 38: 824-844.

Base editing technology, as described herein, can be used to achieve multiplex base editing. For example, in some embodiments, a method of multiplex base editing, as described herein, may comprise: (i) providing a cell, and (ii) introducing into the cell (a) one or more guide RNAs (gRNAs) that target CD33 (Siglec-3), CLL-1, CD123, CD327 (Siglec-6), and/or CD312 (EMR2); (b) one or more gRNAs that target CD33 (Siglec-3), CLL-1, CD123, CD327 (Siglec-6), and/or CD312 (EMR2); and (c) a base editor that binds the one or more gRNAs, wherein the one or more gRNAs are configured to provide an editing event within different target domains, thereby producing a genetically engineered cell. In particular, multiplex base editing can be used to modify one or more target lineage-specific cell-surface antigen (e.g., CD33 (Siglec-3), CLL-1, CD123, CD327 (Siglec-6), and/or CD312 (EMR2)) gene. In particular, multiplex base editing can be used to modify a plurality of target lineage-specific cell-surface antigen (e.g., CD33 (Siglec-3), CLL-1, CD123, CD327 (Siglec-6), and/or CD312 (EMR2)) genes. In particular, multiplex base editing can be used without any risk of translocations. In certain embodiments, multiplex base editing may comprise comboplexing by utilizing a base editor and a CRISPR nuclease without any risk of translocations, for example, a CRISPR nuclease including a Cas9 or a Cas12a nuclease.

In some embodiments, the Cas9 molecule is a base editor. In some embodiments, a base editor is used to a create a genomic modification resulting in a loss of expression of a lineage-specific cell-surface antigen (e.g., CD33 (Siglec-3), CLL-1, CD123, CD327 (Siglec-6), and/or CD312 (EMR2)), or in expression of a lineage-specific cell-surface antigen variant not targeted by an immunotherapy. In some embodiments, a base editor is used to a create a genomic modification resulting in a loss of expression of CD33, or in expression of a CD33 variant not targeted by an immunotherapy. In some embodiments, a base editor is used to a create a genomic modification resulting in a loss of expression ofCLL-1, or in expression of a CLL-1 variant not targeted by an immunotherapy. In some embodiments, a base editor is used to a create a genomic modification resulting in a loss of expression of CD123, or in expression of a CD123 variant not targeted by an immunotherapy. In some embodiments, a base editor is used to a create a genomic modification resulting in a loss of expression of CD327 (Siglec-6), or in expression of a CD327 (Siglec-6) variant not targeted by an immunotherapy. In some embodiments, a base editor is used to a create a genomic modification resulting in a loss of expression of CD312 (EMR2), or in expression of a CD312 (EMR2) variant not targeted by an immunotherapy.

In some embodiments, a base editor is used to create an editing event (e.g., a create a genomic modification) that reduces the activity of a lineage-specific cell-surface antigen (e.g., CD33 (Siglec-3), CLL-1, CD123, CD327 (Siglec-6), and/or CD312 (EMR2)) in a cell.

In some embodiments, a base editor is used to create an editing event (e.g., a create a genomic modification) that reduces the expression level of a nucleic acid encoding a lineage-specific cell-surface antigen (e.g., CD33 (Siglec-3), CLL-1, CD123, CD327 (Siglec-6), and/or CD312 (EMR2)) in a cell.

In some embodiments, a base editor is used to create an editing event (e.g., a create a genomic modification) that abolishes the expression of a full-length lineage-specific cell-surface antigen (e.g., CD33 (Siglec-3), CLL-1, CD123, CD327 (Siglec-6), and/or CD312 (EMR2)) RNA in a cell. In some embodiments, a base editor is used to create an editing event (e.g., a create a genomic modification) that abolishes the expression of a full-length lineage-specific cell-surface antigen (e.g., CD33 (Siglec-3), CLL-1, CD123, CD327 (Siglec-6), and/or CD312 (EMR2)) protein in a cell.

In some embodiments, the cell expresses a truncated version of the lineage-specific cell-surface antigen (e.g., CD33 (Siglec-3), CLL-1, CD123, CD327 (Siglec-6), and/or CD312 (EMR2)) RNA. In some embodiments, the cell expresses a truncated version of the lineage-specific cell-surface antigen (e.g., CD33 (Siglec-3), CLL-1, CD123, CD327 (Siglec-6), and/or CD312 (EMR2)) protein.

In some embodiments, the truncated version of the lineage-specific cell-surface antigen (e.g., CD33 (Siglec-3), CLL-1, CD123, CD327 (Siglec-6), and/or CD312 (EMR2)) RNA is expressed at a level equal to or greater than a level of a full-length lineage-specific cell-surface antigen (e.g., CD33 (Siglec-3), CLL-1, CD123, CD327 (Siglec-6), and/or CD312 (EMR2)) RNA in a non-edited cell. In some embodiments, the truncated version of the lineage-specific cell-surface antigen (e.g., CD33 (Siglec-3), CLL-1, CD123, CD327 (Siglec-6), and/or CD312 (EMR2)) protein is expressed at a level equal to or greater than a level of a full-length lineage-specific cell-surface antigen (e.g., CD33 (Siglec-3), CLL-1, CD123, CD327 (Siglec-6), and/or CD312 (EMR2)) protein in a non-edited cell.

In some embodiments, wherein a function or an activity of the truncated version of the lineage-specific cell-surface antigen (e.g., CD33 (Siglec-3), CLL-1, CD123, CD327 (Siglec-6), and/or CD312 (EMR2)) RNA is impaired or abolished. In some embodiments, wherein a function or an activity of the truncated version of the lineage-specific cell-surface antigen (e.g., CD33 (Siglec-3), CLL-1, CD123, CD327 (Siglec-6), and/or CD312 (EMR2)) protein is impaired or abolished. In some embodiments, a function or an activity of the truncated version of the lineage-specific cell-surface antigen (e.g., CD33 (Siglec-3), CLL-1, CD123, CD327 (Siglec-6), and/or CD312 (EMR2)) RNA that is impaired or abolished comprises binding to an antibody or a chimeric antigen receptor (CAR).

Base editor endonuclease generally comprises a catalytically inactive Cas9 molecule fused to a functional domain, e.g., a deaminase domain. See, e.g., Eid et al. Biochem. J. (2018) 475(11): 1955-1964; Rees et al. Nature Reviews Genetics (2018) 19:770-788. In some embodiments, the catalytically inactive Cas9 molecule is referred to as “dead Cas” or “dCas9.” In some embodiments, the catalytically inactive Cas molecule has reduced activity and is, e.g., a nickase (referred to as “nCas”). In some embodiments, the endonuclease comprises a dCas9 fused to one or more uracil glycosylase inhibitor (UGI) domains. In some embodiments, the endonuclease comprises a dCas9 fused to an adenine base editor (ABE), for example an ABE evolved from the RNA adenine deaminase TadA. In some embodiments, the endonuclease comprises a dCas9 fused to cytidine deaminase enzyme (e.g., APOBEC deaminase, pmCDA1, activation-induced cytidine deaminase (AID)). In some embodiments, the catalytically inactive Cas9 molecule has reduced activity and is nCas9. In some embodiments, the catalytically inactive Cas9 molecule (dCas9) is fused to one or more uracil glycosylase inhibitor (UGI) domains. In some embodiments, the Cas9 molecule comprises an inactive Cas9 molecule (dCas9) fused to an adenine base editor (ABE), for example an ABE evolved from the RNA adenine deaminase TadA. In some embodiments, the Cas9 molecule comprises a nCas9 fused to an adenine base editor (ABE), for example an ABE evolved from the RNA adenine deaminase TadA. In some embodiments, the Cas9 molecule comprises a dCas9 fused to cytidine deaminase enzyme (e.g., APOBEC deaminase, pmCDA1, activation-induced cytidine deaminase (AID)). In some embodiments, the Cas9 molecule comprises a nCas9 fused to cytidine deaminase enzyme (e.g., APOBEC deaminase, pmCDA1, activation-induced cytidine deaminase (AID)).

In some embodiments, the base editor is a cytosine base editor (CBE). In some embodiments, the CBE is CBE1, CBE2, CBE3, or CBE4. In some embodiments, the CBE is selected from the group consisting of nCas9-2×UGI; BE4-rAPOBEC1; BE4-rAPOBEC1 K34A H122A; BE4-PpAPOBEC1; BE4-PpAPOBEC1 R33A; BE4-PpAPOBEC1 H122A; BE4-RrA3F; BE4-AmAPOBEC1; and BE4-SsAPOBEC3B.

In some embodiments, the base editor is an adenine base editor (ABE). In some embodiments, the ABE is ABE1, ABE2, ABE3, ABE4, ABE5, ABE6, ABE7, or ABE8. In some embodiments, the ABE is selected from the group consisting of ABE7.10-m; ABE7.10-d; ABE8.8-m; ABE8.8-d; ABE8.13-m; ABE8.13-d; ABE8.17-m; ABE8.17-d; ABE8.20-m; and ABE8.20-d.

In some embodiments, the base editors includes, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, xBE3, eA3A-BE3, BE-PLUS, TAM, CRISPR-X, ABE7.9, ABE7.10, ABE7.10*, xABE, ABESa, VQR-ABE, VRER-ABE, Sa(KKH)-ABE, and CRISPR-SKIP.

Additional examples of base editors can be found, for example, in US Publication No. 2018/0312825A1, US Publication No. 2018/0312828A1, PCT Publication No. WO 2018/165629A1, Yu et al. Nat Commun. (2020) 11(1):2052, and Gaudelli et al. Nat Biotechnol. (2020) 38(7):892-900. which are incorporated by reference herein in their entireties.

An exemplary abe8_20m sequence is provided below:
atgtccgaagtcgagttttcccatgagtactggatgagacacgca
ttgactctcgcaaagagggctcgagatgaacgcgaggtgcccgtg
ggggcagtactcgtgctcaacaatcgcgtaatcggcgaaggttgg
aatagggcaatcggactccacgaccccactgcacatgcggaaatc
atggcccttcgacagggagggcttgtgatgcagaattatcgactt
tatgatgcgacgctgtactcgacgtttgaaccttgcgtaatgtgc
gcgggagctatgattcactcccgcattggacgagttgtattcggt
gttcgcaacgccaagacgggtgccgcaggttcactgatggacgtg
ctgcatcatccaggcatgaaccaccgggtagaaatcacagaaggc
atattggcggacgaatgtgcggcgctgttgtgtcgtttttttcgc
atgcccaggcgggtctttaacgcccagaaaaaagcacaatcctct
actgactctggtggttcttctggtggttctagcggcagcgagact
cccgggacctcagagtccgccacacccgaaagttctggtggttct
tctggtggttctgacaagaagtacagcatcggcctggccatcggc
accaactctgtgggctgggccgtgatcaccgacgagtacaaggtg
cccagcaagaaattcaaggtgctgggcaacaccgaccggcacagc
atcaagaagaacctgatcggagccctgctgttcgacagcggcgaa
acagccgaggccacccggctgaagagaaccgccagaagaagatac
accagacggaagaaccggatctgctatctgcaagagatcttcagc
aacgagatggccaaggtggacgacagcttcttccacagactggaa
gagtccttcctggtggaagaggataagaagcacgagcggcacccc
atcttcggcaacatcgtggacgaggtggcctaccacgagaagtac
cccaccatctaccacctgagaaagaaactggtggacagcaccgac
aaggccgacctgcggctgatctatctggccctggcccacatgatc
aagttccggggccacttcctgatcgagggcgacctgaaccccgac
aacagcgacgtggacaagctgttcatccagctggtgcagacctac
aaccagctgttcgaggaaaaccccatcaacgccagcggcgtggac
gccaaggccatcctgtctgccagactgagcaagagcagacggctg
gaaaatctgatcgcccagctgcccggcgagaagaagaatggcctg
ttcggaaacctgattgccctgagcctgggcctgacccccaacttc
aagagcaacttcgacctggccgaggatgccaaactgcagctgagc
aaggacacctacgacgacgacctggacaacctgctggcccagatc
ggcgaccagtacgccgacctgtttctggccgccaagaacctgtcc
gacgccatcctgctgagcgacatcctgagagtgaacaccgagatc
accaaggcccccctgagcgcctctatgatcaagagatacgacgag
caccaccaggacctgaccctgctgaaagctctcgtgcggcagcag
ctgcctgagaagtacaaagagattttcttcgaccagagcaagaac
ggctacgccggctacattgacggcggagccagccaggaagagttc
tacaagttcatcaagcccatcctggaaaagatggacggcaccgag
gaactgctcgtgaagctgaacagagaggacctgctgcggaagcag
cggaccttcgacaacggcagcatcccccaccagatccacctggga
gagctgcacgccattctgcggcggcaggaagatttttacccattc
ctgaaggacaaccgggaaaagatcgagaagatcctgaccttccgc
atcccctactacgtgggccctctggccaggggaaacagcagattc
gcctggatgaccagaaagagcgaggaaaccatcaccccctggaac
ttcgaggaagtggtggacaagggcgcttccgcccagagcttcatc
gagcggatgaccaacttcgataagaacctgcccaacgagaaggtg
ctgcccaagcacagcctgctgtacgagtacttcaccgtgtataac
gagctgaccaaagtgaaatacgtgaccgagggaatgagaaagccc
gccttcctgagcggcgagcagaaaaaggccatcgtggacctgctg
ttcaagaccaaccggaaagtgaccgtgaagcagctgaaagaggac
tacttcaagaaaatcgagtgcttcgactccgtggaaatctccggc
gtggaagatcggttcaacgcctccctgggcacataccacgatctg
ctgaaaattatcaaggacaaggacttcctggacaatgaggaaaac
gaggacattctggaagatatcgtgctgaccctgacactgtttgag
gacagagagatgatcgaggaacggctgaaaacctatgcccacctg
ttcgacgacaaagtgatgaagcagctgaagcggcggagatacacc
ggctggggcaggctgagccggaagctgatcaacggcatccgggac
aagcagtccggcaagacaatcctggatttcctgaagtccgacggc
ttcgccaacagaaacttcatgcagctgatccacgacgacagcctg
acctttaaagaggacatccagaaagcccaggtgtccggccagggc
gatagcctgcacgagcacattgccaatctggccggcagccccgcc
attaagaagggcatcctgcagacagtgaaggtggtggacgagctc
gtgaaagtgatgggccggcacaagcccgagaacatcgtgatcgaa
atggccagagagaaccagaccacccagaagggacagaagaacagc
cgcgagagaatgaagcggatcgaagagggcatcaaagagctgggc
agccagatcctgaaagaacaccccgtggaaaacacccagctgcag
aacgagaagctgtacctgtactacctgcagaatgggcgggatatg
tacgtggaccaggaactggacatcaaccggctgtccgactacgat
gtggaccatatcgtgcctcagagctttctgaaggacgactccatc
gacaacaaggtgctgaccagaagcgacaagaaccggggcaagagc
gacaacgtgccctccgaagaggtcgtgaagaagatgaagaactac
tggcggcagctgctgaacgccaagctgattacccagagaaagttc
gacaatctgaccaaggccgagagaggcggcctgagcgaactggat
aaggccggcttcatcaagagacagctggtggaaacccggcagatc
acaaagcacgtggcacagatcctggactcccggatgaacactaag
tacgacgagaatgacaagctgatccgggaagtgaaagtgatcacc
ctgaagtccaagctggtgtccgatttccggaaggatttccagttt
tacaaagtgcgcgagatcaacaactaccaccacgcccacgacgcc
tacctgaacgccgtcgtgggaaccgccctgatcaaaaagtaccct
aagctggaaagcgagttcgtgtacggcgactacaaggtgtacgac
gtgcggaagatgatcgccaagagcgagcaggaaatcggcaaggct
accgccaagtacttcttctacagcaacatcatgaactttttcaag
accgagattaccctggccaacggcgagatccggaagcggcctctg
atcgagacaaacggcgaaaccggggagatcgtgtgggataagggc
cgggattttgccaccgtgcggaaagtgctgagcatgccccaagtg
aatatcgtgaaaaagaccgaggtgcagacaggcggcttcagcaaa
gagtctatcctgcccaagaggaacagcgataagctgatcgccaga
aagaaggactgggaccctaagaagtacggcggcttcgacagcccc
accgtggcctattctgtgctggtggtggccaaagtggaaaagggc
aagtccaagaaactgaagagtgtgaaagagctgctggggatcacc
atcatggaaagaagcagcttcgagaagaatcccatcgactttctg
gaagccaagggctacaaagaagtgaaaaaggacctgatcatcaag
ctgcctaagtactccctgttcgagctggaaaacggccggaagaga
atgctggcctctgccggcgaactgcagaagggaaacgaactggcc
ctgccctccaaatatgtgaacttcctgtacctggccagccactat
gagaagctgaagggctcccccgaggataatgagcagaaacagctg
tttgtggaacagcacaagcactacctggacgagatcatcgagcag
atcagcgagttctccaagagagtgatcctggccgacgctaatctg
gacaaagtgctgtccgcctacaacaagcaccgggataagcccatc
agagagcaggccgagaatatcatccacctgtttaccctgaccaat
ctgggagcccctgccgccttcaagtactttgacaccaccatcgac
cggaagaggtacaccagcaccaaagaggtgctggacgccaccctg
atccaccagagcatcaccggcctgtacgagacacggatcgacctg
tctcagctgggaggtgacgagggagctgataagcgcaccgccgat
ggttccgagttcgaaagccccaagaagaagaggaaagtc
An exemplary ppabobec1-orf-r33a sequence
is provided below:
atgacctctgagaagggccctagcacaggcgaccccaccctgcgg
cggagaatcgagagctgggagttcgacgtgttctacgaccctaga
gaactggccaaggaaacctgcctgctgtacgagatcaagtggggc
atgagcagaaagatctggcggagctctggcaagaacaccaccaac
cacgtggaagtgaatttcatcaagaagttcaccagcgagagaagg
ttccacagcagcatcagctgcagcatcacctggttcctgagctgg
tccccttgctgggaatgcagccaggccatcagagagttcctgagc
caacaccccggagtgacactggtgatctacgtggccagactgttc
tggcacatggaccagagaaacagacagggcctgagagatctggtc
aacagcggcgtgactatccagatcatgcgggccagcgagtactac
cactgttggcggaacttcgtgaactacccccccggcgatgaggcc
cactggcctcagtaccctcctctgtggatgatgctgtacgccctg
gaactgcactgcatcatcctgtctctgcctccatgtctgaagatc
tctagaagatggcagaaccacctggccttcttcagactgcacctg
cagaattgccactaccagaccatccccccccacatcctgctggct
acaggcctgatccacccttctgtgacctggagacttaagagcgga
ggatctagcggcggctctagcggatctgagacacctggcacaagc
gagtctgccacacctgagagtagcggcggatcttctggtggctct
gacaagaagtacagcatcggcctggccatcggcaccaactctgtg
ggctgggccgtgatcaccgacgagtacaaggtgcccagcaagaaa
ttcaaggtgctgggcaacaccgaccggcacagcatcaagaagaac
ctgatcggagccctgctgttcgacagcggcgaaacagccgaggcc
acccggctgaagagaaccgccagaagaagatacaccagacggaag
aaccggatctgctatctgcaagagatcttcagcaacgagatggcc
aaggtggacgacagcttcttccacagactggaagagtccttcctg
gtggaagaggataagaagcacgagcggcaccccatcttcggcaac
atcgtggacgaggtggcctaccacgagaagtaccccaccatctac
cacctgagaaagaaactggtggacagcaccgacaaggccgacctg
cggctgatctatctggccctggcccacatgatcaagttccggggc
cacttcctgatcgagggcgacctgaaccccgacaacagcgacgtg
gacaagctgttcatccagctggtgcagacctacaaccagctgttc
gaggaaaaccccatcaacgccagcggcgtggacgccaaggccatc
ctgtctgccagactgagcaagagcagacggctggaaaatctgatc
gcccagctgcccggcgagaagaagaatggcctgttcggaaacctg
attgccctgagcctgggcctgacccccaacttcaagagcaacttc
gacctggccgaggatgccaaactgcagctgagcaaggacacctac
gacgacgacctggacaacctgctggcccagatcggcgaccagtac
gccgacctgtttctggccgccaagaacctgtccgacgccatcctg
ctgagcgacatcctgagagtgaacaccgagatcaccaaggccccc
ctgagcgcctctatgatcaagagatacgacgagcaccaccaggac
ctgaccctgctgaaagctctcgtgcggcagcagctgcctgagaag
tacaaagagattttcttcgaccagagcaagaacggctacgccggc
tacattgacggcggagccagccaggaagagttctacaagttcatc
aagcccatcctggaaaagatggacggcaccgaggaactgctcgtg
aagctgaacagagaggacctgctgcggaagcagcggaccttcgac
aacggcagcatcccccaccagatccacctgggagagctgcacgcc
attctgcggcggcaggaagatttttacccattcctgaaggacaac
cgggaaaagatcgagaagatcctgaccttccgcatcccctactac
gtgggccctctggccaggggaaacagcagattcgcctggatgacc
agaaagagcgaggaaaccatcaccccctggaacttcgaggaagtg
gtggacaagggcgcttccgcccagagcttcatcgagcggatgacc
aacttcgataagaacctgcccaacgagaaggtgctgcccaagcac
agcctgctgtacgagtacttcaccgtgtataacgagctgaccaaa
gtgaaatacgtgaccgagggaatgagaaagcccgccttcctgagc
ggcgagcagaaaaaggccatcgtggacctgctgttcaagaccaac
cggaaagtgaccgtgaagcagctgaaagaggactacttcaagaaa
atcgagtgcttcgactccgtggaaatctccggcgtggaagatcgg
ttcaacgcctccctgggcacataccacgatctgctgaaaattatc
aaggacaaggacttcctggacaatgaggaaaacgaggacattctg
gaagatatcgtgctgaccctgacactgtttgaggacagagagatg
atcgaggaacggctgaaaacctatgcccacctgttcgacgacaaa
gtgatgaagcagctgaagcggcggagatacaccggctggggcagg
ctgagccggaagctgatcaacggcatccgggacaagcagtccggc
aagacaatcctggatttcctgaagtccgacggcttcgccaacaga
aacttcatgcagctgatccacgacgacagcctgacctttaaagag
gacatccagaaagcccaggtgtccggccagggcgatagcctgcac
gagcacattgccaatctggccggcagccccgccattaagaagggc
atcctgcagacagtgaaggtggtggacgagctcgtgaaagtgatg
ggccggcacaagcccgagaacatcgtgatcgaaatggccagagag
aaccagaccacccagaagggacagaagaacagccgcgagagaatg
aagcggatcgaagagggcatcaaagagctgggcagccagatcctg
aaagaacaccccgtggaaaacacccagctgcagaacgagaagctg
tacctgtactacctgcagaatgggcgggatatgtacgtggaccag
gaactggacatcaaccggctgtccgactacgatgtggaccatatc
gtgcctcagagctttctgaaggacgactccatcgacaacaaggtg
ctgaccagaagcgacaagaaccggggcaagagcgacaacgtgccc
tccgaagaggtcgtgaagaagatgaagaactactggcggcagctg
ctgaacgccaagctgattacccagagaaagttcgacaatctgacc
aaggccgagagaggcggcctgagcgaactggataaggccggcttc
atcaagagacagctggtggaaacccggcagatcacaaagcacgtg
gcacagatcctggactcccggatgaacactaagtacgacgagaat
gacaagctgatccgggaagtgaaagtgatcaccctgaagtccaag
ctggtgtccgatttccggaaggatttccagttttacaaagtgcgc
gagatcaacaactaccaccacgcccacgacgcctacctgaacgcc
gtcgtgggaaccgccctgatcaaaaagtaccctaagctggaaagc
gagttcgtgtacggcgactacaaggtgtacgacgtgcggaagatg
atcgccaagagcgagcaggaaatcggcaaggctaccgccaagtac
ttcttctacagcaacatcatgaactttttcaagaccgagattacc
ctggccaacggcgagatccggaagcggcctctgatcgagacaaac
ggcgaaaccggggagatcgtgtgggataagggccgggattttgcc
accgtgcggaaagtgctgagcatgccccaagtgaatatcgtgaaa
aagaccgaggtgcagacaggcggcttcagcaaagagtctatcctg
cccaagaggaacagcgataagctgatcgccagaaagaaggactgg
gaccctaagaagtacggcggcttcgacagccccaccgtggcctat
tctgtgctggtggtggccaaagtggaaaagggcaagtccaagaaa
ctgaagagtgtgaaagagctgctggggatcaccatcatggaaaga
agcagcttcgagaagaatcccatcgactttctggaagccaagggc
tacaaagaagtgaaaaaggacctgatcatcaagctgcctaagtac
tccctgttcgagctggaaaacggccggaagagaatgctggcctct
gccggcgaactgcagaagggaaacgaactggccctgccctccaaa
tatgtgaacttcctgtacctggccagccactatgagaagctgaag
ggctcccccgaggataatgagcagaaacagctgtttgtggaacag
cacaagcactacctggacgagatcatcgagcagatcagcgagttc
tccaagagagtgatcctggccgacgctaatctggacaaagtgctg
tccgcctacaacaagcaccgggataagcccatcagagagcaggcc
gagaatatcatccacctgtttaccctgaccaatctgggagcccct
gccgccttcaagtactttgacaccaccatcgaccggaagaggtac
accagcaccaaagaggtgctggacgccaccctgatccaccagagc
atcaccggcctgtacgagacacggatcgacctgtctcagctggga
ggtgactctggtggaagcggaggatctggcggcagcaccaatctg
agcgacatcatcgagaaagagacaggcaagcagctggtcatccaa
gagtccatcctgatgctgcctgaagaggtggaagaagtgatcggc
aacaagcccgagtccgacatcctggtgcacaccgcctacgatgag
agcaccgacgagaacgtgatgctgctgacctctgacgcccctgag
tacaagccttgggctctcgtgatccaggacagcaacggcgagaac
aagatcaagatgctgagcggcggctctggtggctctggcggatct
acaaacctgtccgatattattgagaaagaaaccgggaaacagctc
gtgattcaagagtctattctcatgctcccggaagaagtcgaggaa
gtcattggaaacaagcctgagagcgatattctggtccatacagcc
tacgacgagtctaccgatgagaatgtcatgctcctcaccagcgac
gctcccgagtataagccatgggcacttgtcattcaggactccaat
ggggaaaacaaaatcaaaatgctcccaaagaaaaaacgcaaggtg
An exemplary ppabobec1-orf-wt sequence is
provided below:
atgacctctgagaagggccctagcacaggcgaccccaccctgcgg
cggagaatcgagagctgggagttcgacgtgttctacgaccctaga
gaactgagaaaggaaacctgcctgctgtacgagatcaagtggggc
atgagcagaaagatctggcggagctctggcaagaacaccaccaac
cacgtggaagtgaatttcatcaagaagttcaccagcgagagaagg
ttccacagcagcatcagctgcagcatcacctggttcctgagctgg
tccccttgctgggaatgcagccaggccatcagagagttcctgagc
caacaccccggagtgacactggtgatctacgtggccagactgttc
tggcacatggaccagagaaacagacagggcctgagagatctggtc
aacagcggcgtgactatccagatcatgcgggccagcgagtactac
cactgttggcggaacttcgtgaactacccccccggcgatgaggcc
cactggcctcagtaccctcctctgtggatgatgctgtacgccctg
gaactgcactgcatcatcctgtctctgcctccatgtctgaagatc
tctagaagatggcagaaccacctggccttcttcagactgcacctg
cagaattgccactaccagaccatccccccccacatcctgctggct
acaggcctgatccacccttctgtgacctggagacttaagagcgga
ggatctagcggcggctctagcggatctgagacacctggcacaagc
gagtctgccacacctgagagtagcggcggatcttctggtggctct
gacaagaagtacagcatcggcctggccatcggcaccaactctgtg
ggctgggccgtgatcaccgacgagtacaaggtgcccagcaagaaa
ttcaaggtgctgggcaacaccgaccggcacagcatcaagaagaac
ctgatcggagccctgctgttcgacagcggcgaaacagccgaggcc
acccggctgaagagaaccgccagaagaagatacaccagacggaag
aaccggatctgctatctgcaagagatcttcagcaacgagatggcc
aaggtggacgacagcttcttccacagactggaagagtccttcctg
gtggaagaggataagaagcacgagcggcaccccatcttcggcaac
atcgtggacgaggtggcctaccacgagaagtaccccaccatctac
cacctgagaaagaaactggtggacagcaccgacaaggccgacctg
cggctgatctatctggccctggcccacatgatcaagttccggggc
cacttcctgatcgagggcgacctgaaccccgacaacagcgacgtg
gacaagctgttcatccagctggtgcagacctacaaccagctgttc
gaggaaaaccccatcaacgccagcggcgtggacgccaaggccatc
ctgtctgccagactgagcaagagcagacggctggaaaatctgatc
gcccagctgcccggcgagaagaagaatggcctgttcggaaacctg
attgccctgagcctgggcctgacccccaacttcaagagcaacttc
gacctggccgaggatgccaaactgcagctgagcaaggacacctac
gacgacgacctggacaacctgctggcccagatcggcgaccagtac
gccgacctgtttctggccgccaagaacctgtccgacgccatcctg
ctgagcgacatcctgagagtgaacaccgagatcaccaaggccccc
ctgagcgcctctatgatcaagagatacgacgagcaccaccaggac
ctgaccctgctgaaagctctcgtgcggcagcagctgcctgagaag
tacaaagagattttcttcgaccagagcaagaacggctacgccggc
tacattgacggcggagccagccaggaagagttctacaagttcatc
aagcccatcctggaaaagatggacggcaccgaggaactgctcgtg
aagctgaacagagaggacctgctgcggaagcagcggaccttcgac
aacggcagcatcccccaccagatccacctgggagagctgcacgcc
attctgcggcggcaggaagatttttacccattcctgaaggacaac
cgggaaaagatcgagaagatcctgaccttccgcatcccctactac
gtgggccctctggccaggggaaacagcagattcgcctggatgacc
agaaagagcgaggaaaccatcaccccctggaacttcgaggaagtg
gtggacaagggcgcttccgcccagagcttcatcgagcggatgacc
aacttcgataagaacctgcccaacgagaaggtgctgcccaagcac
agcctgctgtacgagtacttcaccgtgtataacgagctgaccaaa
gtgaaatacgtgaccgagggaatgagaaagcccgccttcctgagc
ggcgagcagaaaaaggccatcgtggacctgctgttcaagaccaac
cggaaagtgaccgtgaagcagctgaaagaggactacttcaagaaa
atcgagtgcttcgactccgtggaaatctccggcgtggaagatcgg
ttcaacgcctccctgggcacataccacgatctgctgaaaattatc
aaggacaaggacttcctggacaatgaggaaaacgaggacattctg
gaagatatcgtgctgaccctgacactgtttgaggacagagagatg
atcgaggaacggctgaaaacctatgcccacctgttcgacgacaaa
gtgatgaagcagctgaagcggcggagatacaccggctggggcagg
ctgagccggaagctgatcaacggcatccgggacaagcagtccggc
aagacaatcctggatttcctgaagtccgacggcttcgccaacaga
aacttcatgcagctgatccacgacgacagcctgacctttaaagag
gacatccagaaagcccaggtgtccggccagggcgatagcctgcac
gagcacattgccaatctggccggcagccccgccattaagaagggc
atcctgcagacagtgaaggtggtggacgagctcgtgaaagtgatg
ggccggcacaagcccgagaacatcgtgatcgaaatggccagagag
aaccagaccacccagaagggacagaagaacagccgcgagagaatg
aagcggatcgaagagggcatcaaagagctgggcagccagatcctg
aaagaacaccccgtggaaaacacccagctgcagaacgagaagctg
tacctgtactacctgcagaatgggcgggatatgtacgtggaccag
gaactggacatcaaccggctgtccgactacgatgtggaccatatc
gtgcctcagagctttctgaaggacgactccatcgacaacaaggtg
ctgaccagaagcgacaagaaccggggcaagagcgacaacgtgccc
tccgaagaggtcgtgaagaagatgaagaactactggcggcagctg
ctgaacgccaagctgattacccagagaaagttcgacaatctgacc
aaggccgagagaggcggcctgagcgaactggataaggccggcttc
atcaagagacagctggtggaaacccggcagatcacaaagcacgtg
gcacagatcctggactcccggatgaacactaagtacgacgagaat
gacaagctgatccgggaagtgaaagtgatcaccctgaagtccaag
ctggtgtccgatttccggaaggatttccagttttacaaagtgcgc
gagatcaacaactaccaccacgcccacgacgcctacctgaacgcc
gtcgtgggaaccgccctgatcaaaaagtaccctaagctggaaagc
gagttcgtgtacggcgactacaaggtgtacgacgtgcggaagatg
atcgccaagagcgagcaggaaatcggcaaggctaccgccaagtac
ttcttctacagcaacatcatgaactttttcaagaccgagattacc
ctggccaacggcgagatccggaagcggcctctgatcgagacaaac
ggcgaaaccggggagatcgtgtgggataagggccgggattttgcc
accgtgcggaaagtgctgagcatgccccaagtgaatatcgtgaaa
aagaccgaggtgcagacaggcggcttcagcaaagagtctatcctg
cccaagaggaacagcgataagctgatcgccagaaagaaggactgg
gaccctaagaagtacggcggcttcgacagccccaccgtggcctat
tctgtgctggtggtggccaaagtggaaaagggcaagtccaagaaa
ctgaagagtgtgaaagagctgctggggatcaccatcatggaaaga
agcagcttcgagaagaatcccatcgactttctggaagccaagggc
tacaaagaagtgaaaaaggacctgatcatcaagctgcctaagtac
tccctgttcgagctggaaaacggccggaagagaatgctggcctct
gccggcgaactgcagaagggaaacgaactggccctgccctccaaa
tatgtgaacttcctgtacctggccagccactatgagaagctgaag
ggctcccccgaggataatgagcagaaacagctgtttgtggaacag
cacaagcactacctggacgagatcatcgagcagatcagcgagttc
tccaagagagtgatcctggccgacgctaatctggacaaagtgctg
tccgcctacaacaagcaccgggataagcccatcagagagcaggcc
gagaatatcatccacctgtttaccctgaccaatctgggagcccct
gccgccttcaagtactttgacaccaccatcgaccggaagaggtac
accagcaccaaagaggtgctggacgccaccctgatccaccagagc
atcaccggcctgtacgagacacggatcgacctgtctcagctggga
ggtgactctggtggaagcggaggatctggcggcagcaccaatctg
agcgacatcatcgagaaagagacaggcaagcagctggtcatccaa
gagtccatcctgatgctgcctgaagaggtggaagaagtgatcggc
aacaagcccgagtccgacatcctggtgcacaccgcctacgatgag
agcaccgacgagaacgtgatgctgctgacctctgacgcccctgag
tacaagccttgggctctcgtgatccaggacagcaacggcgagaac
aagatcaagatgctgagcggcggctctggtggctctggcggatct
acaaacctgtccgatattattgagaaagaaaccgggaaacagctc
gtgattcaagagtctattctcatgctcccggaagaagtcgaggaa
gtcattggaaacaagcctgagagcgatattctggtccatacagcc
tacgacgagtctaccgatgagaatgtcatgctcctcaccagcgac
gctcccgagtataagccatgggcacttgtcattcaggactccaat
ggggaaaacaaaatcaaaatgctcccaaagaaaaaacgcaaggtg
An exemplary prtn_SzW8eqL7-abe8_20m sequence
is provided below:
MSEVEFSHEYWMRHALTLAKRARDEREVPVGAVLVLNNRVIGEGW
NRAIGLHDPTAHAEIMALRQGGLVMQNYRLYDATLYSTFEPCVMC
AGAMIHSRIGRVVFGVRNAKTGAAGSLMDVLHHPGMNHRVEITEG
ILADECAALLCRFFRMPRRVFNAQKKAQSSTDSGGSSGGSSGSET
PGTSESATPESSGGSSGGSDKKYSIGLAIGTNSVGWAVITDEYKV
PSKKFKVLGNTDRHSIKKNLIGALLFDSGETAEATRLKRTARRRY
TRRKNRICYLQEIFSNEMAKVDDSFFHRLEESFLVEEDKKHERHP
IFGNIVDEVAYHEKYPTIYHLRKKLVDSTDKADLRLIYLALAHMI
KFRGHFLIEGDLNPDNSDVDKLFIQLVQTYNQLFEENPINASGVD
AKAILSARLSKSRRLENLIAQLPGEKKNGLFGNLIALSLGLTPNF
KSNFDLAEDAKLQLSKDTYDDDLDNLLAQIGDQYADLFLAAKNLS
DAILLSDILRVNTEITKAPLSASMIKRYDEHHQDLTLLKALVRQQ
LPEKYKEIFFDQSKNGYAGYIDGGASQEEFYKFIKPILEKMDGTE
ELLVKLNREDLLRKQRTFDNGSIPHQIHLGELHAILRRQEDFYPF
LKDNREKIEKILTFRIPYYVGPLARGNSRFAWMTRKSEETITPWN
FEEVVDKGASAQSFIERMTNFDKNLPNEKVLPKHSLLYEYFTVYN
ELTKVKYVTEGMRKPAFLSGEQKKAIVDLLFKTNRKVTVKQLKED
YFKKIECFDSVEISGVEDRFNASLGTYHDLLKIIKDKDFLDNEEN
EDILEDIVLTLTLFEDREMIEERLKTYAHLFDDKVMKQLKRRRYT
GWGRLSRKLINGIRDKQSGKTILDFLKSDGFANRNFMQLIHDDSL
TFKEDIQKAQVSGQGDSLHEHIANLAGSPAIKKGILQTVKVVDEL
VKVMGRHKPENIVIEMARENQTTQKGQKNSRERMKRIEEGIKELG
SQILKEHPVENTQLQNEKLYLYYLQNGRDMYVDQELDINRLSDYD
VDHIVPQSFLKDDSIDNKVLTRSDKNRGKSDNVPSEEVVKKMKNY
WRQLLNAKLITQRKFDNLTKAERGGLSELDKAGFIKRQLVETRQI
TKHVAQILDSRMNTKYDENDKLIREVKVITLKSKLVSDFRKDFQF
YKVREINNYHHAHDAYLNAVVGTALIKKYPKLESEFVYGDYKVYD
VRKMIAKSEQEIGKATAKYFFYSNIMNFFKTEITLANGEIRKRPL
IETNGETGEIVWDKGRDFATVRKVLSMPQVNIVKKTEVQTGGFSK
ESILPKRNSDKLIARKKDWDPKKYGGFDSPTVAYSVLVVAKVEKG
KSKKLKSVKELLGITIMERSSFEKNPIDFLEAKGYKEVKKDLIIK
LPKYSLFELENGRKRMLASAGELQKGNELALPSKYVNFLYLASHY
EKLKGSPEDNEQKQLFVEQHKHYLDEIIEQISEFSKRVILADANL
DKVLSAYNKHRDKPIREQAENIIHLFTLTNLGAPAAFKYFDTTID
RKRYTSTKEVLDATLIHQSITGLYETRIDLSQLGGDEGADKRTAD
GSEFESPKKKRKV
An exemplary prtn_ZJVPExXY-ppabobec1-r33a-
protein sequence is provided below:
MTSEKGPSTGDPTLRRRIESWEFDVFYDPRELAKETCLLYEIKWG
MSRKIWRSSGKNTTNHVEVNFIKKFTSERRFHSSISCSITWFLSW
SPCWECSQAIREFLSQHPGVTLVIYVARLFWHMDQRNRQGLRDLV
NSGVTIQIMRASEYYHCWRNFVNYPPGDEAHWPQYPPLWMMLYAL
ELHCIILSLPPCLKISRRWQNHLAFFRLHLQNCHYQTIPPHILLA
TGLIHPSVTWRLKSGGSSGGSSGSETPGTSESATPESSGGSSGGS
DKKYSIGLAIGTNSVGWAVITDEYKVPSKKFKVLGNTDRHSIKKN
LIGALLFDSGETAEATRLKRTARRRYTRRKNRICYLQEIFSNEMA
KVDDSFFHRLEESFLVEEDKKHERHPIFGNIVDEVAYHEKYPTIY
HLRKKLVDSTDKADLRLIYLALAHMIKFRGHFLIEGDLNPDNSDV
DKLFIQLVQTYNQLFEENPINASGVDAKAILSARLSKSRRLENLI
AQLPGEKKNGLFGNLIALSLGLTPNFKSNFDLAEDAKLQLSKDTY
DDDLDNLLAQIGDQYADLFLAAKNLSDAILLSDILRVNTEITKAP
LSASMIKRYDEHHQDLTLLKALVRQQLPEKYKEIFFDQSKNGYAG
YIDGGASQEEFYKFIKPILEKMDGTEELLVKLNREDLLRKQRTFD
NGSIPHQIHLGELHAILRRQEDFYPFLKDNREKIEKILTFRIPYY
VGPLARGNSRFAWMTRKSEETITPWNFEEVVDKGASAQSFIERMT
NFDKNLPNEKVLPKHSLLYEYFTVYNELTKVKYVTEGMRKPAFLS
GEQKKAIVDLLFKTNRKVTVKQLKEDYFKKIECFDSVEISGVEDR
FNASLGTYHDLLKIIKDKDFLDNEENEDILEDIVLTLTLFEDREM
IEERLKTYAHLFDDKVMKQLKRRRYTGWGRLSRKLINGIRDKQSG
KTILDFLKSDGFANRNFMQLIHDDSLTFKEDIQKAQVSGQGDSLH
EHIANLAGSPAIKKGILQTVKVVDELVKVMGRHKPENIVIEMARE
NQTTQKGQKNSRERMKRIEEGIKELGSQILKEHPVENTQLQNEKL
YLYYLQNGRDMYVDQELDINRLSDYDVDHIVPQSFLKDDSIDNKV
LTRSDKNRGKSDNVPSEEVVKKMKNYWRQLLNAKLITQRKFDNLT
KAERGGLSELDKAGFIKRQLVETRQITKHVAQILDSRMNTKYDEN
DKLIREVKVITLKSKLVSDFRKDFQFYKVREINNYHHAHDAYLNA
VVGTALIKKYPKLESEFVYGDYKVYDVRKMIAKSEQEIGKATAKY
FFYSNIMNFFKTEITLANGEIRKRPLIETNGETGEIVWDKGRDFA
TVRKVLSMPQVNIVKKTEVQTGGFSKESILPKRNSDKLIARKKDW
DPKKYGGFDSPTVAYSVLVVAKVEKGKSKKLKSVKELLGITIMER
SSFEKNPIDFLEAKGYKEVKKDLIIKLPKYSLFELENGRKRMLAS
AGELQKGNELALPSKYVNFLYLASHYEKLKGSPEDNEQKQLFVEQ
HKHYLDEIIEQISEFSKRVILADANLDKVLSAYNKHRDKPIREQA
ENIIHLFTLTNLGAPAAFKYFDTTIDRKRYTSTKEVLDATLIHQS
ITGLYETRIDLSQLGGDSGGSGGSGGSTNLSDIIEKETGKQLVIQ
ESILMLPEEVEEVIGNKPESDILVHTAYDESTDENVMLLTSDAPE
YKPWALVIQDSNGENKIKMLSGGSGGSGGSTNLSDIIEKETGKQL
VIQESILMLPEEVEEVIGNKPESDILVHTAYDESTDENVMLLTSD
APEYKPWALVIQDSNGENKIKMLPKKKRKV
An exemplary prtn_ZyqE8AYc-ppabobec1-wt-protein
sequence is provided below:
MTSEKGPSTGDPTLRRRIESWEFDVFYDPRELRKETCLLYEIKWG
MSRKIWRSSGKNTTNHVEVNFIKKFTSERRFHSSISCSITWFLSW
SPCWECSQAIREFLSQHPGVTLVIYVARLFWHMDQRNRQGLRDLV
NSGVTIQIMRASEYYHCWRNFVNYPPGDEAHWPQYPPLWMMLYAL
ELHCIILSLPPCLKISRRWQNHLAFFRLHLQNCHYQTIPPHILLA
TGLIHPSVTWRLKSGGSSGGSSGSETPGTSESATPESSGGSSGGS
DKKYSIGLAIGTNSVGWAVITDEYKVPSKKFKVLGNTDRHSIKKN
LIGALLFDSGETAEATRLKRTARRRYTRRKNRICYLQEIFSNEMA
KVDDSFFHRLEESELVEEDKKHERHPIFGNIVDEVAYHEKYPTIY
HLRKKLVDSTDKADLRLIYLALAHMIKFRGHFLIEGDLNPDNSDV
DKLFIQLVQTYNQLFEENPINASGVDAKAILSARLSKSRRLENLI
AQLPGEKKNGLFGNLIALSLGLTPNFKSNFDLAEDAKLQLSKDTY
DDDLDNLLAQIGDQYADLFLAAKNLSDAILLSDILRVNTEITKAP
LSASMIKRYDEHHQDLTLLKALVRQQLPEKYKEIFFDQSKNGYAG
YIDGGASQEEFYKFIKPILEKMDGTEELLVKLNREDLLRKQRTFD
NGSIPHQIHLGELHAILRRQEDFYPFLKDNREKIEKILTFRIPYY
VGPLARGNSRFAWMTRKSEETITPWNFEEVVDKGASAQSFIERMT
NFDKNLPNEKVLPKHSLLYEYFTVYNELTKVKYVTEGMRKPAFLS
GEQKKAIVDLLFKTNRKVTVKQLKEDYFKKIECFDSVEISGVEDR
FNASLGTYHDLLKIIKDKDFLDNEENEDILEDIVLTLTLFEDREM
IEERLKTYAHLFDDKVMKQLKRRRYTGWGRLSRKLINGIRDKQSG
KTILDFLKSDGFANRNFMQLIHDDSLTFKEDIQKAQVSGQGDSLH
EHIANLAGSPAIKKGILQTVKVVDELVKVMGRHKPENIVIEMARE
NQTTQKGQKNSRERMKRIEEGIKELGSQILKEHPVENTQLQNEKL
YLYYLQNGRDMYVDQELDINRLSDYDVDHIVPQSFLKDDSIDNKV
LTRSDKNRGKSDNVPSEEVVKKMKNYWRQLLNAKLITQRKFDNLT
KAERGGLSELDKAGFIKRQLVETRQITKHVAQILDSRMNTKYDEN
DKLIREVKVITLKSKLVSDFRKDFQFYKVREINNYHHAHDAYLNA
VVGTALIKKYPKLESEFVYGDYKVYDVRKMIAKSEQEIGKATAKY
FFYSNIMNFFKTEITLANGEIRKRPLIETNGETGEIVWDKGRDFA
TVRKVLSMPQVNIVKKTEVQTGGFSKESILPKRNSDKLIARKKDW
DPKKYGGFDSPTVAYSVLVVAKVEKGKSKKLKSVKELLGITIMER
SSFEKNPIDFLEAKGYKEVKKDLIIKLPKYSLFELENGRKRMLAS
AGELQKGNELALPSKYVNFLYLASHYEKLKGSPEDNEQKQLFVEQ
HKHYLDEIIEQISEFSKRVILADANLDKVLSAYNKHRDKPIREQA
ENIIHLFTLTNLGAPAAFKYFDTTIDRKRYTSTKEVLDATLIHQS
ITGLYETRIDLSQLGGDSGGSGGSGGSTNLSDIIEKETGKQLVIQ
ESILMLPEEVEEVIGNKPESDILVHTAYDESTDENVMLLTSDAPE
YKPWALVIQDSNGENKIKMLSGGSGGSGGSTNLSDIIEKETGKQL
VIQESILMLPEEVEEVIGNKPESDILVHTAYDESTDENVMLLTSD
APEYKPWALVIQDSNGENKIKMLPKKKRKV

In some embodiments, the base editor has been further modified to inhibit base excision repair at the target site and induce cellular mismatch repair. Any of the Cas9 molecules described herein may be fused to a Gam domain (bacteriophage Mu protein) to protect the Cas9 molecule from degradation and exonuclease activity. See, e.g., Eid et al. Biochem. J. (2018) 475(11): 1955-1964.

In some embodiments, the Cas9 molecule belongs to class 2 type V of Cas endonuclease. Class 2 type V Cas endonucleases can be further categorized as type V-A, type V-B, type V-C, and type V-U. See, e.g., Stella et al. Nature Structural & Molecular Biology (2017) 24: 882-892. In some embodiments, the Cas molecule is a type V-A Cas endonuclease, such as a Cpf1 (Cas12a) nuclease. In some embodiments, the Cas9 molecule is a type V-B Cas endonuclease, such as a C2c1 endonuclease. See, e.g., Shmakov et al. Mol Cell (2015) 60: 385-397. In some embodiments, the Cas molecule is MAD7™. Alternatively or in addition, the Cas9 molecule is a Cpf1 nuclease or a variant thereof. As will be appreciated by one of skill in the art, the Cpf1 nuclease may also be referred to as Cas12a. See, e.g., Strohkendl et al. Mol. Cell (2018) 71: 1-9. In some embodiments, a composition or method described herein involves, or a host cell expresses a Cpf1 nuclease derived from Provetella spp. or Francisella spp., Acidaminococcus sp. (AsCpf1), Lachnospiraceae bacterium (LpCpf1), or Eubacterium rectale. In some embodiments, the nucleotide sequence encoding the Cpf1 nuclease may be codon optimized for expression in a host cell. In some embodiments, the nucleotide sequence encoding the Cpf1 endonuclease is further modified to alter the activity of the protein.

Both naturally occurring and modified variants of CRISPR/Cas nucleases are suitable for use according to aspects of this disclosure. For example, dCas or nickase variants, Cas variants having altered PAM specificities, and Cas variants having improved nuclease activities are embraced by some embodiments of this disclosure. In some embodiments, catalytically inactive variants of Cas molecules (e.g., of Cas9 or Cas12a) are used according to the methods described herein. A catalytically inactive variant of Cpf1 (Cas12a) may be referred to dCas12a. As described herein, catalytically inactive variants of Cpf1 maybe fused to a function domain to form a base editor. See, e.g., Rees et al. Nature Reviews Genetics (2018) 19:770-788. In some embodiments, the catalytically inactive Cas9 molecule is dCas9. In some embodiments, the endonuclease comprises a dCas12a fused to one or more uracil glycosylase inhibitor (UGI) domains. In some embodiments, the Cas9 molecule comprises a dCas12a fused to an adenine base editor (ABE), for example an ABE evolved from the RNA adenine deaminase TadA. In some embodiments, the Cas molecule comprises a dCas12a fused to cytidine deaminase enzyme (e.g., APOBEC deaminase, pmCDA1, activation-induced cytidine deaminase (AID)).

### Zinc Finger Nucleases

In some embodiments, a cell or cell population described herein is produced using zinc finger (ZFN) technology. In some embodiments, the ZFN recognizes a target domain described herein, e.g., a target domain in CD33 (Siglec-3), CLL-1, CD123, CD327 (Siglec-6), and/or CD312 (EMR2) described herein. In general, zinc finger mediated genomic editing involves use of a zinc finger nuclease, which typically comprises a zinc finger DNA binding domain and a nuclease domain. The zinc finger binding domain may be engineered to recognize and bind to any target domain of interest, e.g., may be designed to recognize a DNA sequence ranging from about 3 nucleotides to about 21 nucleotides in length, or from about 8 to about 19 nucleotides in length. Zinc finger binding domains typically comprise at least three zinc finger recognition regions (e.g., zinc fingers).

Restriction endonucleases (restriction enzymes) capable of sequence-specific binding to DNA (at a recognition site) and cleaving DNA at or near the site of binding are known in the art and may be used to form ZFN for use in genomic editing. For example, Type IIS restriction endonucleases cleave DNA at sites removed from the recognition site and have separable binding and cleavage domains. In one example, the DNA cleavage domain may be derived from the FokI endonuclease.

### TALENs

In some embodiments, a cell or cell population described herein is produced using TALEN technology. In some embodiments, the TALEN recognizes a target domain described herein, e.g., a target domain in CD33 (Siglec-3), CLL-1, CD123, CD327 (Siglec-6), and/or CD312 (EMR2) described herein. In general, TALENs are engineered restriction enzymes that can specifically bind and cleave a desired target DNA molecule. A TALEN typically contains a Transcriptional Activator-Like Effector (TALE) DNA-binding domain fused to a DNA cleavage domain. The DNA binding domain may contain a highly conserved 33-34 amino acid sequence with a divergent 2 amino acid RVD (repeat variable dipeptide motif) at positions 12 and 13. The RVD motif determines binding specificity to a nucleic acid sequence and can be engineered to specifically bind a desired DNA sequence. In one example, the DNA cleavage domain may be derived from the FokI endonuclease. In some embodiments, the FokI domain functions as a dimer, using two constructs with unique DNA binding domains for sites in the target genome with proper orientation and spacing.

A TALEN specific to a target gene of interest can be used inside a cell to produce a double-stranded break (DSB). A mutation can be introduced at the break site if the repair mechanisms improperly repair the break via non-homologous end joining. For example, improper repair may introduce a frame shift mutation. Alternatively, a foreign DNA molecule having a desired sequence can be introduced into the cell along with the TALEN. Depending on the sequence of the foreign DNA and chromosomal sequence, this process can be used to correct a defect or introduce a DNA fragment into a target gene of interest, or introduce such a defect into the endogenous gene, thus decreasing expression of the target gene.

Some exemplary, non-limiting embodiments of endonucleases and nuclease variants suitable for use in connection with the guide RNAs and genetic engineering methods provided herein have been described above. Additional suitable nucleases and nuclease variants will be apparent to those of skill in the art based on the present disclosure and the knowledge in the art. The disclosure is not limited in this respect.

gRNA Sequences and Configurations

gRNA configuration generally

A gRNA can comprise a number of domains. In an embodiment, a unimolecular, sgRNA, or chimeric, gRNA comprises, e.g., from 5′ to 3′:
a targeting domain (which is complementary, or partially complementary, to a target nucleic acid sequence in a target gene, e.g., in the lineage-specific cell-surface antigen (e.g., CD33 (Siglec-3), CLL-1, CD123, CD327 (Siglec-6), and/or CD312 (EMR2)) gene;
a first complementarity domain;
a linking domain;
a second complementarity domain (which is complementary to the first complementarity domain);
a proximal domain; and
optionally, a tail domain.

Each of these domains is now described in more detail.

The targeting domain may comprise a nucleotide sequence that is complementary, e.g., at least 80, 85, 90, or 95% complementary, e.g., fully complementary, to the target sequence on the target nucleic acid. The targeting domain is part of an RNA molecule and will therefore typically comprise the base uracil (U), while any DNA encoding the gRNA molecule will comprise the base thymine (T). While not wishing to be bound by theory, in an embodiment, it is believed that the complementarity of the targeting domain with the target sequence contributes to specificity of the interaction of the gRNA/Cas9 molecule complex with a target nucleic acid. It is understood that in a targeting domain and target sequence pair, the uracil bases in the targeting domain will pair with the adenine bases in the target sequence. In an embodiment, the target domain itself comprises in the 5′ to 3′ direction, an optional secondary domain, and a core domain. In an embodiment, the core domain is fully complementary with the target sequence. In an embodiment, the targeting domain is 5 to 50 nucleotides in length. The targeting domain may be between 15 and 30 nucleotides, 15-25 nucleotides, 18-22 nucleotides, or 19-21 nucleotides in length. In some embodiments, the targeting domain is 15, 16, 17, 18, 19, 20, 21, 22, 23, 24, or 25 nucleotides in length. In some embodiments, the targeting domain is between 10-30, or between 15-25, nucleotides in length. The targeting domain corresponds fully with the target domain sequence (i.e., without any mismatch nucleotides), or may comprise one or more, but typically not more than 4, mismatches. As the targeting domain is part of an RNA molecule, the gRNA, it will typically comprise ribonucleotides, while the DNA targeting domain will comprise deoxyribonucleotides.

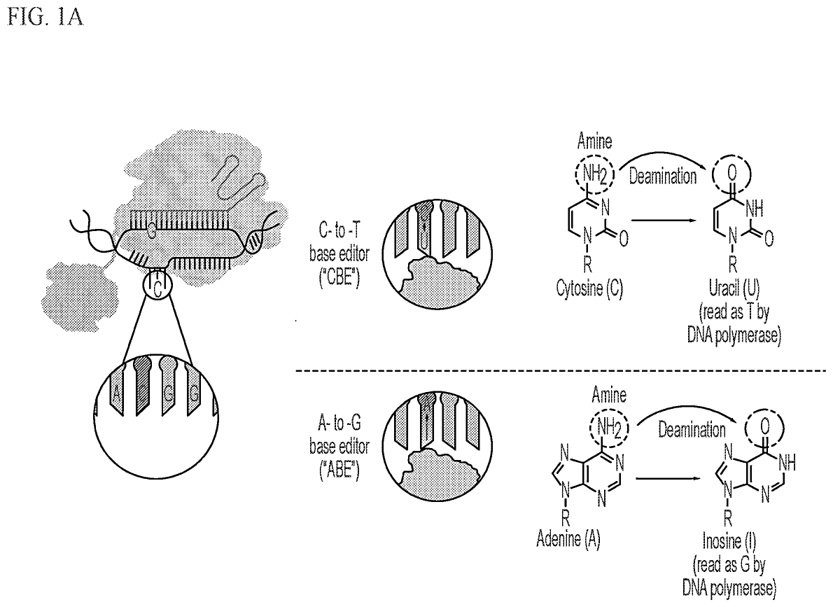

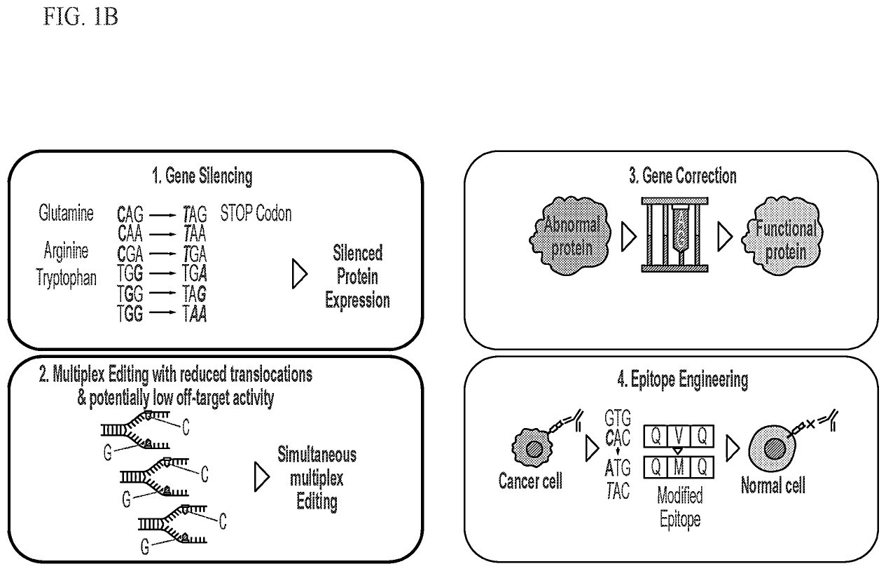

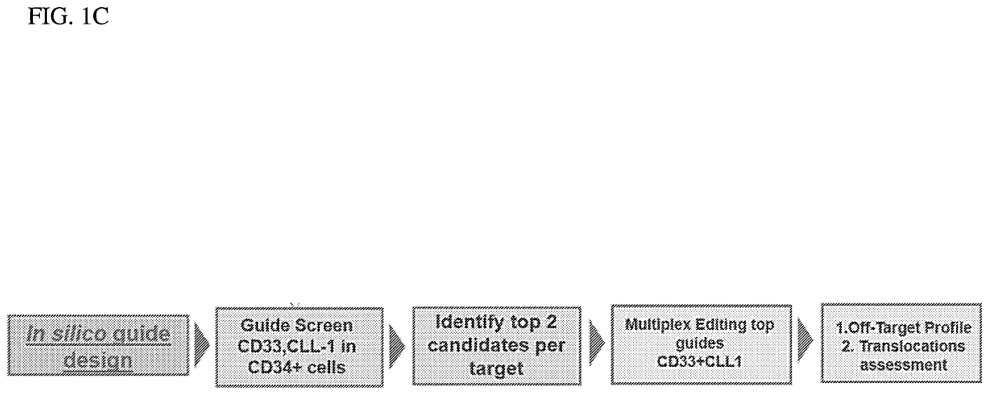

The targeting domain of the gRNA thus base-pairs (in full or partial complementarity) with the sequence of the double-stranded target site that is complementary to the sequence of the target domain, and thus with the strand complementary to the strand that comprises the PAM sequence. It will be understood that the targeting domain of the gRNA typically does not include the PAM sequence. It will further be understood that the location of the PAM may be 5′ or 3′ of the target domain sequence, depending on the nuclease employed. For example, the PAM is typically 3′ of the target domain sequences for Cas9 nucleases, and 5′ of the target domain sequence for Cas12a nucleases. For an illustration of the location of the PAM and the mechanism of gRNA binding a target site, see, e.g., FIG. 1 of Vanegas et al., Fungal Biol Biotechnol. 2019; 6: 6, which is incorporated by reference herein. For additional illustration and description of the mechanism of gRNA targeting an RNA-guided nuclease to a target site, see Fu Y et al, Nat Biotechnol 2014 (doi: 10.1038/nbt.2808) and Sternberg SH et al., Nature 2014 (doi: 10.1038/naturel3011), both incorporated herein by reference.

An exemplary illustration of a Cas9 target site, comprising a 22 nucleotide target domain, and an NGG PAM sequence, as well as of a gRNA comprising a targeting domain that fully corresponds to the target domain (and thus base-pairs with full complementarity with the DNA strand complementary to the strand comprising the target domain and PAM) is provided below:

```
      [ target domain (DNA) ][ PAM ]
5′-N-N-N-N-N-N-N-N-N-N-N-N-N-N-N-N-N-N-N-N-N-N-N-G-G-3′ (DNA)
3′-N-N-N-N-N-N-N-N-N-N-N-N-N-N-N-N-N-N-N-N-N-N-N-C-C-5′ (DNA)
      | | | | | | | | | | | | | | | | | | | | | |
5′-N-N-N-N-N-N-N-N-N-N-N-N-N-N-N-N-N-N-N-N-N-N-[gRNA scaffold]-3′ (RNA)
    [ targeting domain (RNA) ] [binding domain]
```

An exemplary illustration of a Cas12a target site, comprising a 22 nucleotide target domain, and a TTN PAM sequence, as well as of a gRNA comprising a targeting domain that fully corresponds to the target domain (and thus base-pairs with full complementarity with the DNA strand complementary to the strand comprising the target domain and PAM) is provided below:

```
                        [ PAM ][ target domain (DNA) ]
                    5′-T-T-N-N-N-N-N-N-N-N-N-N-N-N-N-N-N-N-N-N-N-N-N-N-N-3′ (DNA)
                    3′-A-A-N-N-N-N-N-N-N-N-N-N-N-N-N-N-N-N-N-N-N-N-N-N-N-5′ (DNA)
                                      | | | | | | | | | | | | | | | | | | | | | |
5′-[gRNA scaffold]-N-N-N-N-N-N-N-N-N-N-N-N-N-N-N-N-N-N-N-N-N-N-3′ (RNA)
    [binding domain] [ targeting domain (RNA) ]
```

In some embodiments, the Cas12a PAM sequence is 5′-T-T-T-V-3′.

While not wishing to be bound by theory, at least in some embodiments, it is believed that the length and complementarity of the targeting domain with the target sequence contributes to specificity of the interaction of the gRNA/Cas9 molecule complex with a target nucleic acid. In some embodiments, the targeting domain of a gRNA provided herein is 5 to 50 nucleotides in length. In some embodiments, the targeting domain is 15 to 25 nucleotides in length. In some embodiments, the targeting domain is 18 to 22 nucleotides in length. In some embodiments, the targeting domain is 19-21 nucleotides in length. In some embodiments, the targeting domain is 15 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In some embodiments, the targeting domain fully corresponds, without mismatch, to a target domain sequence provided herein, or a part thereof. In some embodiments, the targeting domain of a gRNA provided herein comprises 1 mismatch relative to a target domain sequence provided herein. In some embodiments, the targeting domain comprises 2 mismatches relative to the target domain sequence. In some embodiments, the target domain comprises 3 mismatches relative to the target domain sequence.

In some embodiments, a targeting domain comprises a core domain and a secondary targeting domain, e.g., as described in PCT Publication No. WO 2015/157070, which is incorporated by reference in its entirety. In some embodiments, the core domain comprises about 8 to about 13 nucleotides from the 3′ end of the targeting domain (e.g., the most 3′ 8 to 13 nucleotides of the targeting domain). In an embodiment, the secondary domain is positioned 5′ to the core domain. In many embodiments, the core domain has exact complementarity (corresponds fully) with the corresponding region of the target sequence, or part thereof. In other embodiments, the core domain can have 1 or more nucleotides that are not complementary (mismatched) with the corresponding nucleotide of the target domain sequence.

The first complementarity domain is complementary with the second complementarity domain, and in an embodiment, has sufficient complementarity to the second complementarity domain to form a duplexed region under at least some physiological conditions. In an embodiment, the first complementarity domain is 5 to 30 nucleotides in length. In an embodiment, the first complementarity domain comprises 3 subdomains, which, in the 5′ to 3′ direction are: a 5′ subdomain, a central subdomain, and a 3′ subdomain. In an embodiment, the 5′ subdomain is 4 to 9, e.g., 4, 5, 6, 7, 8 or 9 nucleotides in length. In an embodiment, the central subdomain is 1, 2, or 3, e.g., 1, nucleotide in length. In an embodiment, the 3′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. The first complementarity domain can share homology with, or be derived from, a naturally occurring first complementarity domain. In an embodiment, it has at least 50% homology with a S. pyogenes, S. aureus or S. thermophilus, first complementarity domain.

The sequence and placement of the above-mentioned domains are described in more detail in PCT Publication No. WO2015/157070, which is herein incorporated by reference in its entirety, including p. 88-112 therein.

A linking domain serves to link the first complementarity domain with the second complementarity domain of a unimolecular gRNA. The linking domain can link the first and second complementarity domains covalently or non-covalently. In an embodiment, the linkage is covalent. In an embodiment, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in PCT Publication No. WO2018/126176, the entire contents of which are incorporated herein by reference.

The second complementarity domain is complementary, at least in part, with the first complementarity domain, and in an embodiment, has sufficient complementarity to the second complementarity domain to form a duplexed region under at least some physiological conditions. In an embodiment, the second complementarity domain can include a sequence that lacks complementarity with the first complementarity domain, e.g., a sequence that loops out from the duplexed region. In an embodiment, the second complementarity domain is 5 to 27 nucleotides in length. In an embodiment, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24 or 25 nucleotides in length. In an embodiment, the second complementarity domain comprises 3 subdomains, which, in the 5′ to 3′ direction are: a 5′ subdomain, a central subdomain, and a 3′ subdomain. In an embodiment, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In an embodiment, the central subdomain is 1, 2, 3, 4 or 5, e.g., 3, nucleotides in length. In an embodiment, the 3′ subdomain is 4 to 9, e.g., 4, 5, 6, 7, 8 or 9 nucleotides in length. In an embodiment, the 5′ subdomain and the 3′ subdomain of the first complementarity domain, are respectively, complementary, e.g., fully complementary, with the 3′ subdomain and the 5′ subdomain of the second complementarity domain.

In an embodiment, the proximal domain is 5 to 20 nucleotides in length. In an embodiment, the proximal domain can share homology with or be derived from a naturally occurring proximal domain. In an embodiment, it has at least 50% homology with an S. pyogenes, S. aureus or S. thermophilus, proximal domain.

A broad spectrum of tail domains are suitable for use in gRNAs. In an embodiment, the tail domain is 0 (absent), 1, 2, 3, 4, 5, 6, 7, 8, 9, or 10 nucleotides in length. In some embodiments, the tail domain nucleotides are from or share homology with a sequence from the 5′ end of a naturally occurring tail domain. In some embodiments, the tail domain includes sequences that are complementary to each other and which, under at least some physiological conditions, form a duplexed region. In some embodiments, the tail domain is absent or is 1 to 50 nucleotides in length. In some embodiments, the tail domain can share homology with or be derived from a naturally occurring proximal tail domain. In some embodiments, it has at least 50% homology with an S. pyogenes, S. aureus or S. thermophilus, tail domain. In an embodiment, the tail domain includes nucleotides at the 3′ end that are related to the method of in vitro or in vivo transcription.

In some embodiments, modular gRNA comprises:
a first strand comprising, e.g., from 5′ to 3′:
   a targeting domain (which is complementary to a target nucleic acid in the lineage-specific cell-surface antigen (e.g., CD33 (Siglec-3), CLL-1, CD123, CD327 (Siglec-6), and/or CD312 (EMR2)) gene) and a first complementarity domain; and
a second strand, comprising, preferably from 5′ to 3′:
   optionally, a 5′ extension domain;
   a second complementarity domain;
   a proximal domain; and
   optionally, a tail domain.

In some embodiments, the gRNA is chemically modified. In some embodiments, any of the gRNAs provided herein comprise one or more nucleotides that are chemically modified. Chemical modifications of gRNAs have previously been described, and suitable chemical modifications include any modifications that are beneficial for gRNA function and do not measurably increase any undesired characteristics, e.g., off-target effects, of a given gRNA. Suitable chemical modifications include, for example, those that make a gRNA less susceptible to endo- or exonuclease catalytic activity, and include, without limitation, that the gRNA may comprise one or more modification chosen from phosphorothioate backbone modification, 2′-O-Me-modified sugars (e.g., at one or both of the 3′ and 5′ termini), 2′F-modified sugar, replacement of the ribose sugar with the bicyclic nucleotide-cEt, 3′thioPACE (MSP), or any combination thereof. Additional suitable gRNA modifications will be apparent to the skilled artisan based on this disclosure, and such suitable gRNA modification include, without limitation, those described, e.g., in Rahdar et al. PNAS Dec. 22, 2015 112 (51) E7110-E7117 and Hendel et al., Nat Biotechnol. 2015 Sep.; 33(9): 985-989, each of which is incorporated herein by reference in its entirety. In some embodiments, a gRNA described herein comprises one or more 2′-O-methyl-3′-phosphorothioate nucleotides, e.g., at least 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at the three terminal positions and the 5′ end and/or at the three terminal positions and the 3′ end. In some embodiments, the gRNA may comprise one or more modified nucleotides, e.g., as described in PCT Publication Nos. WO2017/214460, WO2016/089433, and WO2016/164356, which are incorporated by reference their entirety.

In some embodiments, a gRNA described herein is chemically modified. For example, the gRNA may comprise one or more 2′-O modified nucleotides, e.g., 2′-O-methyl nucleotides. In some embodiments, the gRNA comprises a 2′-O modified nucleotide, e.g., 2′-O-methyl nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g. 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g. 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, the gRNA may comprise one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous-modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, the gRNA may comprise one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O-modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g. 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, the gRNA comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, the gRNA comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

Some exemplary, non-limiting embodiments of modifications, e.g., chemical modifications, suitable for use in connection with the guide RNAs and genetic engineering methods provided herein have been described above. Additional suitable modifications, e.g., chemical modifications, will be apparent to those of skill in the art based on the present disclosure and the knowledge in the art, including, but not limited to those described in Hendel, A. et al., Nature Biotech., 2015, Vol 33, No. 9; in PCT Publication No. WO2017/214460; WO2016/089433; and in WO2016/164356; each one of which is herein incorporated by reference in its entirety.

The lineage-specific cell-surface antigen (e.g., CD33 (Siglec-3), CLL-1, CD123, CD327 (Siglec-6), and/or CD312 (EMR2)) targeting gRNAs provided herein can be delivered to a cell in any manner suitable. Various suitable methods for the delivery of CRISPR/Cas systems, e.g., comprising an ribonucleoprotein (RNP) complex including a gRNA bound to an RNA-guided nuclease, have been described, and exemplary suitable methods include, without limitation, electroporation of an RNP into a cell, electroporation of mRNA encoding a Cas nuclease and a gRNA into a cell, various protein or nucleic acid transfection methods, and delivery of encoding RNA or DNA via viral vectors, such as, for example, retroviral (e.g., lentiviral) vectors. Any suitable delivery method is embraced by this disclosure, and the disclosure is not limited in this respect.

gRNAs Targeting CD33 (Siglec-3)

The present disclosure provides a number of useful gRNAs that can target an endonuclease to human CD33. In some embodiments, the gRNA is a gRNA disclosed in any of PCT Publication Nos. WO2017/066760, WO2020/047164, WO2020/150478, and WO2020/237217, WO2019/046285, WO/2018/160768, or Borot et al. PNAS Jun. 11, 2019 116 (24) 11978-11987, each of which is incorporated herein by reference in its entirety. Table 1-3 and Table A below illustrates target domains in human endogenous CD33 that can be bound by gRNAs described herein.

TABLE 1
Exemplary target domains of human CD33 bound by various gRNAs are described herein. For each
target domain, the first sequence represents an exemplary 20-nucleotide DNA sequence
corresponding to the target domain sequence that can be targeted by a suitable gRNA, which may
comprise an equivalent RNA targeting domain sequence (comprising RNA nucleotides instead of DNA
nucleotides), and the second sequence is the reverse complement thereof.

| Identifier | gRNA Type | Target Domain Sequence | SEQ ID NO. | Reverse Compliment | SEQ ID NO. |
|---|---|---|---|---|---|
| CD33_CBEsg11 | iSTOP guide | ACCTGTCAGGTGAAGTTCGC | 1 | GCGAACTTCACCTGACAGGT | 17 |
| CD33_CBEsg7 | iSTOP guide | ACAGGCCCAGGACACAGAGC | 2 | GCTCTGTGTCCTGGGCCTGT | 18 |
| CD33_CBEsg9 | iSTOP guide | TGACAACCAGGAGAAGATCG | 3 | CGATCTTCTCCTGGTTGTCA | 19 |
| CD33_CBEsg12 | iSTOP guide | GTTCCACAGAACCCAACAAC | 4 | GTTGTTGGGTTCTGTGGAAC | 20 |
| CD33_CBEsg8 | iSTOP guide | GACAACCAGGAGAAGATCGG | 5 | CCGATCTTCTCCTGGTTGTC | 21 |
| CD33_CBEsg10 | iSTOP guide | CTGACAACCAGGAGAAGATC | 6 | GATCTTCTCCTGGTTGTCAG | 22 |
| CD33_ABEsg13 | SpliceR guide | TTCCTACCTGAGCCATCTCC | 7 | GGAGATGGCTCAGGTAGGAA | 23 |
| CD33_ABEsg14 | SpliceR guide | ACTTACAGGTGACGTTGAGC | 8 | GCTCAACGTCACCTGTAAGT | 24 |
| CD33_ABEsg15 | SpliceR guide | CCACTCACCTGCCCACAGCA | 9 | TGCTGTGGGCAGGTGAGTGG | 25 |
| CD33_ABEsg16 | SpliceR guide | ATGCTCACATGAAGAAGATG | 10 | CATCTTCTTCATGTGAGCAT | 26 |
| CD33_ABEsg17 | SpliceR guide | CACTCACCTGCCCACAGCAG | 11 | CATCTTCTTCATGTGAGCAT | 27 |

TABLE 2
Exemplary target domain sequences of human CD33 bound by various gRNAs are provided herein.
For each target domain, the first sequence represents a DNA target domain sequence and the
second sequence represents an exemplary equivalent gRNA targeting domain sequence.

| | | | SEQ | gRNA Targeting Domain | SEQ |
|---|---|---|---|---|---|
| Identifier | gRNA Type | Target Domain Sequence | ID NO. | Sequence | ID NO. |
| CD33_CBEsg11 | iSTOP guide | ACCTGTCAGGTGAAGTTCGC | 1 | ACCUGUCAGGUGAAGUUCGC | 33 |
| CD33_CBEsg7 | iSTOP guide | ACAGGCCCAGGACACAGAGC | 2 | ACAGGCCCAGGACACAGAGC | 34 |
| CD33_CBEsg9 | iSTOP guide | TGACAACCAGGAGAAGATCG | 3 | UGACAACCAGGAGAAGAUCG | 35 |
| CD33_CBEsg12 | iSTOP guide | GTTCCACAGAACCCAACAAC | 4 | GUUCCACAGAACCCAACAAC | 36 |
| CD33_CBEsg8 | iSTOP guide | GACAACCAGGAGAAGATCGG | 5 | GACAACCAGGAGAAGAUCGG | 37 |
| CD33_CBEsg10 | iSTOP guide | CTGACAACCAGGAGAAGATC | 6 | CUGACAACCAGGAGAAGAUC | 38 |
| CD33_ABEsg13 | SpliceR guide | TTCCTACCTGAGCCATCTCC | 7 | UUCCUACCUGAGCCAUCUCC | 39 |
| CD33_ABEsg14 | SpliceR guide | ACTTACAGGTGACGTTGAGC | 8 | ACUUACAGGUGACGUUGAGC | 40 |
| CD33_ABEsg15 | SpliceR guide | CCACTCACCTGCCCACAGCA | 9 | CCACUCACCUGCCCACAGCA | 41 |
| CD33_ABEsg16 | SpliceR guide | ATGCTCACATGAAGAAGATG | 10 | AUGCUCACAUGAAGAAGAUG | 42 |
| CD33_ABEsg17 | SpliceR guide | CACTCACCTGCCCACAGCAG | 11 | CACUCACCUGCCCACAGCAG | 43 |

TABLE 3
Exemplary target domain sequences of human CD33 bound by various gRNAs
are provided herein. For each target domain, a DNA target sequence in
the human CD33 genomic sequence is provided. A gRNA targeting a target
domain provided herein may comprise an equivalent RNA sequence within
its targeting domain.

| Identifier | Target Gene | Target Domain Sequence | SEQ ID NO. |
|---|---|---|---|
| CD33_CBEsg11 | CD33 | ACCTGTCAGGTGAAGTTCGC | 1 |
| CD33_CBEsg7 | CD33 | ACAGGCCCAGGACACAGAGC | 2 |
| CD33_CBEsg9 | CD33 | TGACAACCAGGAGAAGATCG | 3 |
| CD33_CBEsg12 | CD33 | GTTCCACAGAACCCAACAAC | 4 |
| CD33_CBEsg8 | CD33 | GACAACCAGGAGAAGATCGG | 5 |
| CD33_CBEsg10 | CD33 | CTGACAACCAGGAGAAGATC | 6 |
| CD33_ABEsg13 | CD33 | TTCCTACCTGAGCCATCTCC | 7 |
| CD33_ABEsg14 | CD33 | ACTTACAGGTGACGTTGAGC | 8 |
| CD33_ABEsg15 | CD33 | CCACTCACCTGCCCACAGCA | 9 |
| CD33_ABEsg16 | CD33 | ATGCTCACATGAAGAAGATG | 10 |
| CD33_ABEsg17 | CD33 | CACTCACCTGCCCACAGCAG | 11 |

TABLE 4
Exemplary human CD33 target sequences. Certain target sequences are
followed by a PAM sequence, indicated by a space in the text. Suitable
gRNAs binding the target sequences provided will typically comprise a
targeting domain comprising an RNA nucleotide sequence equivalent to the
respective target sequence (and excluding the PAM).

| gRNA target | Target sequences | SEQ ID NO: |
|---|---|---|
| hCD33 | ACCTGTCAGGTGAAGTTCGC TGG | 178 |
| hCD33 | TGGCCGGGTTCTAGAGTGCC AGG | 179 |
| hCD33 | GGCCGGGTTCTAGAGTGCCA GGG | 180 |
| hCD33 | CACCGAGGAGTGAGTAGTCC TGG | 181 |
| hCD33 | TCCAGCGAACTTCACCTGAC AGG | 182 |
| CD33 (in intron 1) | GCTGTGGGGAGAGGGGTTGT | 183 |
| CD33 (in intron 1) | CTGTGGGGAGAGGGGTTGTC | 184 |
| CD33 (in intron 1) | TGGGGAAACGAGGGTCAGCT | 185 |
| CD33 (in intron 1) | GGGCCCCTGTGGGGAAACGA | 186 |
| CD33 (in intron 1) | AGGGCCCCTGTGGGGAAACG | 187 |
| CD33 (in intron 1) | GCTGACCCTCGTTTCCCCAC | 188 |
| CD33 (in intron 1) | CTGACCCTCGTTTCCCCACA | 189 |
| CD33 (in intron 1) | TGACCCTCGTTTCCCCACAG | 190 |
| CD33 (in intron 1) | CCATAGCCAGGGCCCCTGTG | 191 |
| CD33 (in intron 2) | GCATGTGACAGGTGAGGCAC | 192 |
| CD33 (in intron 2) | TGAGGCACAGGCTTCAGAAG | 193 |
| CD33 (in intron 2) | AGGCTTCAGAAGTGGCCGCA | 194 |
| CD33 (in intron 2) | GGCTTCAGAAGTGGCCGCAA | 195 |
| CD33 (in intron 2) | GTACCCATGAACTTCCCTTG | 196 |
| CD33 (in intron 2) | GTGGCCGCAAGGGAAGTTCA | 197 |
| CD33 (in intron 2) | TGGCCGCAAGGGAAGTTCAT | 198 |
| CD33 (in intron 2) | GGAAGTTCATGGGTACTGCA | 199 |
| CD33 (in intron 2) | TTCATGGGTACTGCAGGGCA | 200 |
| CD33 (in intron 2) | CTAAACCCCTCCCAGTACCA | 201 |
| CD33 (in intron 1) | CACTCACCTGCCCACAGCAG | 202 |
| CD33 (in intron 1) | CCCTGCTGTGGGCAGGTGAG | 203 |
| CD33 (in intron 1) | TGGGCAGGTGAGTGGCTGTG | 204 |
| CD33 (in intron 1) | GGTGAGTGGCTGTGGGGAGA | 205 |
| CD33 (in intron 1) | GTGAGTGGCTGTGGGGAGAG | 206 |
| CD33 (exon 2) | ATCCATAGCCAGGGCCCCTG | 207 |
| CD33 (exon 2) | TCCATAGCCAGGGCCCCTGT | 208 |
| CD33 (exon 2) | CCATAGCCAGGGCCCCTGTG | 191 |
| CD33 (exon 2) | TCGTTTCCCCACAGGGGCCC | 209 |
| CD33 (exon 2) | TGGCTATGGATCCAAATTTC | 210 |
| CD33 (exon 2) | TGGGGAAACGAGGGTCAGCT | 185 |
| CD33 (exon 2) | GGGCCCCTGTGGGGAAACGA | 186 |
| CD33 (exon 2) | AGAAATTTGGATCCATAGCC AGG | 211 |
| CD33 (exon 3) | ATCCCTGGCACTCTAGAACC CGG | 212 |
| CD33 (exon 3) | CCTCACTAGACTTGACCCAC AGG | 213 |

TABLE 5
Sequences of target domains of human CD33, CD123,
or CLL-1 that can be bound by suitable gRNAs. The
adjacent PAM sequences are also provided. A
suitable gRNA typically comprises a targeting
domain that may comprise an RNA sequence
equivalent to the target domain sequence.

| Target | | | Target |
|---|---|---|---|
| gene | Sequence | PAM | location |
| CD33 | CCCCAGGACTACTCACTCCT | CGG | CD33 |
| | (SEQ ID NO: 64) | | exon 3 |
| CD123 | TTTCTTGAGCTGCAGCTGGG | CGG | CD123 |
| | (SEQ ID NO: 65) | | exon 5 |
| | AGTTCCCACATCCTGGTGCG | GGG | CD123 |
| | (SEQ ID NO: 66) | | exon 6 |
| CLL-1 | GGTGGCTATTGTTTGCAGTG | TGG | CLL-1 |
| | (SEQ ID NO: 74) | | exon 4 |

TABLE 15
Exemplary ABE guides

| | | | | | | gRNA |
|---|---|---|---|---|---|---|
| | Spacer Sequence | SEQ | | | SEQ | Targeting |
| ABE | (I.e., | ID | | Reverse | ID | Domain |
| Guide | Target Domain Sequence) | NO. | PAM | Compliment | NO. | Sequence |
| CD33 | ATGCTCACATGAAG | 2000 | AGG | CATCTTCTTCATGTG | 2002 | TGTCTCTGCC |
| ABE | AAGATG | | | AGCAT | | TCATCTTCTT |
| sg16 | | | | | | CATGTGAGCA |
| | | | | | | TTTTCTCTGG |
| | | | | | | GTCAGGCATG |
| | | | | | | GGCC |
| CD33 | CACTCACCTGCCCA | 2001 | GGG | CTGCTGTGGGCAGGT | 2003 | TGCTACTGCT |
| ABE | CAGCAG | | | GAGTG | | GCCCCTGCTG |
| sg17 | | | | | | TGGGCAGGTG |
| | | | | | | AGTGGCTGTG |
| | | | | | | GGGAGAGGGG |
| | | | | | | TTGTCG |

gRNAs Targeting CLL-1

The present disclosure provides a number of useful gRNAs that can target an endonuclease to human CLL-1. In some embodiments, the gRNA that can target an endonuclease to human CLL-1 is a gRNA disclosed in any of PCT Publication Nos. WO2020/047164 and WO2021/041971, each of which is incorporated herein by reference in its entirety. Table 1 below illustrates target domains in human endogenous CLL-1 that can be bound by gRNAs described herein.

TABLE 6
Exemplary target domains of human CLL-1 bound by various gRNAs are described herein.
For each target domain, the first sequence represents an exemplary 20-nucleotide DNA
sequence corresponding to the target domain sequence that can be targeted by a
suitable gRNA, which may comprise an equivalent RNA targeting domain sequence
(comprising RNA nucleotides instead of DNA nucleotides), and the second sequence is
the reverse complement thereof.

| | | | SEQ | | SEQ |
|---|---|---|---|---|---|
| | | | ID | | ID |
| Identifier | gRNA Type | Target Domain Sequence | NO. | Reverse Compliment | NO. |
| CLL-1_CBEsg3 | iSTOP guide | CAAATCCATCTCCTTGGACA | 12 | TGTCCAAGGAGATGGATTTG | 28 |
| CLL-1_CBEsg4 | iSTOP guide | TTATGCCAAATCCATCTCCT | 13 | AGGAGATGGATTTGGCATAA | 29 |
| CLL-1_CBEsg1 | iSTOP guide | ATTCCAGAACTCCAGTGAGA | 14 | TCTCACTGGAGTTCTGGAAT | 30 |
| CLL-1_CBEsg2 | iSTOP guide | CGCCATACATGAGAGGGAGC | 15 | GCTCCCTCTCATGTATGGCG | 31 |
| CLL-1_CBEsg5 | iSTOP guide | AACATGGCAGGAGAGTAAAA | 16 | TTTTACTCTCCTGCCATGTT | 32 |

TABLE 7
Exemplary target domain sequences of human CLL-1 bound by various gRNAs are provided herein.
For each target domain, the first sequence represents a DNA target domain sequenc in the
human CLL-1 genomic sequence, and the second sequence represents an exemplary equivalent
gRNA targeting domain sequence.

| | | | SEQ | gRNA Targeting Domain | SEQ |
|---|---|---|---|---|---|
| Identifier | gRNA Type | Target Domain Sequence | ID NO. | Sequence | ID NO. |
| CLL-1_CBEsg3 | iSTOP guide | CAAATCCATCTCCTTGGACA | 12 | CAAAUCCAUCUCCUUGGACA | 44 |
| CLL-1_CBEsg4 | iSTOP guide | TTATGCCAAATCCATCTCCT | 13 | UUAUGCCAAAUCCAUCUCCU | 45 |
| CLL-1_CBEsg1 | iSTOP guide | ATTCCAGAACTCCAGTGAGA | 14 | AUUCCAGAACUCCAGUGAGA | 46 |
| CLL-1_CBEsg2 | iSTOP guide | CGCCATACATGAGAGGGAGC | 15 | CGCCAUACAUGAGAGGGAGC | 47 |
| CLL-1_CBEsg5 | iSTOP guide | AACATGGCAGGAGAGTAAAA | 16 | AACAUGGCAGGAGAGUAAAA | 48 |

TABLE 16
Exemplary target domains of human CLL-1 bound by various gRNAs are described herein. For each
target domain, the first sequence represents an exemplary DNA target sequence adjacent to a
suitable PAM in the human CLL-1 genomic sequence, which may comprise an equivalent RNA targeting
domain sequence (comprising RNA nucleotides instead of DNA nucleotides), and the second
sequence is the reverse complement thereof.

| | Spacer Sequence | | | | |
|---|---|---|---|---|---|
| | (i.e., Target Domain | SEQ | | SEQ | gRNA Targeting |
| Guide | Sequence) | ID NO. | Reverse Compliment | ID NO. | Domain Sequence |
| g7 | ACTCACCTATCCACATGCAG | 1100 | CTGCATGTGGATAGGTGAGT | 1135 | ACUCACCUAUCCACAUGCAG |
| g7A | TCAAGATTTCTTTACATATTC | 1101 | GAATATGTAAAGAAATCTTGA | 1136 | UCAAGAUUUCUUUACAUAUUC |
| g8 | GACACTCACCTATCCACATG | 1102 | CATGTGGATAGGTGAGTGTC | 1137 | GACACUCACCUAUCCACAUG |
| g10 | CTCACCTATCCACATGCAGA | 1103 | TCTGCATGTGGATAGGTGAG | 1138 | CUCACCUAUCCACAUGCAGA |
| g11 | CTTCTGCATGTGGATAGGTG | 1104 | CACCTATCCACATGCAGAAG | 1139 | CUUCUGCAUGUGGAUAGGUG |
| g12 | TCTGCATGTGGATAGGTGAG | 1105 | CTCACCTATCCACATGCAGA | 1140 | UCUGCAUGUGGAUAGGUGAG |
| g13 | ACACTCACCTATCCACATGC | 1106 | GCATGTGGATAGGTGAGTGT | 1141 | ACACUCACCUAUCCACAUGC |
| g15 | AAAGGTAAGATTTTGAGTTA | 1107 | TAACTCAAAATCTTACCTTT | 1142 | AAAGGUAAGAUUUUGAGUUA |
| g22 | CACAGCACCTCCAGCTCCCTC | 1108 | GAGGGAGCTGGAGGTGCTGTG | 1143 | CACAGCACCUCCAGCUCCCUC |
| g29 | AATGTGGAGTTCACGTAACTT | 1109 | AAGTTACGTGAACTCCACATT | 1144 | AAUGUGGAGUUCACGUAACUU |
| g30 | ATGTGGAGTTCACGTAACTTT | 1110 | AAAGTTACGTGAACTCCACAT | 1145 | AUGUGGAGUUCACGUAACUUU |
| g38 | AAGGTAATCTTGTATTCTCT | 1111 | AGAGAATACAAGATTACCTT | 1146 | AAGGUAAUCUUGUAUUCUCU |
| g59 | CTGTAGGAATTTATAAAATCC | 1112 | GGATTTTATAAATTCCTACAG | 1147 | CUGUAGGAAUUUAUAAAAUCC |
| g60 | CTTACCTTTTTCCCCAAATT | 1113 | AATTTGGGGAAAAAGGTAAG | 1148 | CUUACCUUUUUCCCCAAAUU |
| g64 | AAGGTAAGATTTTGAGTTAT | 1114 | ATAACTCAAAATCTTACCTT | 1149 | AAGGUAAGAUUUUGAGUUAU |
| g69 | AGTACATACACATGCTTGCC | 1115 | GGCAAGGATGTGTATGTACT | 1150 | AGUACAUACACAUGCUUGCC |
| g69 | TCTTTCAGGGTTATAAGAAAC | 1116 | GTTTCTTATAACCCTGAAAGA | 1151 | UCUUUCAGGGUUAUAAGAAAC |
| g70 | GTGGAGTTCACGTAACTTTG | 1117 | CAAAGTTACGTGAACTCCAC | 1152 | GUGGAGUUCACGUAACUUUG |
| g104 | TTACCTTGTTCTTTGCTATA | 1118 | TATAGCAAAGAACAAGGTAA | 1153 | UUACCUUGUUCUUUGCUAUA |
| g106 | GGTAATCTTGTATTCTCTTG | 1119 | CAAGAGAATACAAGATTACC | 1154 | GGUAAUCUUGUAUUCUCUUG |
| g110 | TTCTTCCAGAGCACAAATGT | 1120 | ACATTTGTGCTCTGGAAGAA | 1155 | UUCUUCCAGAGCACAAAUGU |
| g120 | GTACATACACATGCTTGCCA | 1121 | TGGCAAGCATGTGTATGTAC | 1156 | GUACAUACACAUGCUUGCCA |
| g127 | ATGTGGAGTTCAGGTAACTT | 1122 | AAGTTACGTGAACTCCACAT | 1157 | AUGUGGAGUUCACGUAACUU |
| g128 | TGGAGTTCACGTAACTTTGA | 1123 | TCAAAGTTACGTGAACTCCA | 1158 | UGGAGUUCACGUAACUUUGA |
| g154 | TGTAGGAATTTATAAAATCC | 1124 | GGATTTTATAAATTCCTACA | 1159 | UGUAGGAAUUUAUAAAAUCC |
| g183 | TGGTAAGTGTCTATTCTTGT | 1125 | ACAAGAATAGACACTTACCA | 1160 | UGGUAAGUGUCUAUUCUUGU |
| g197 | AGGTAATCTTGTATTCTCTT | 1126 | AAGAGAATACAAGATTACCT | 1161 | AGGUAAUCUUGUAUUCUCUU |
| g202 | TCTTCCAGAGCACAAATGTA | 1127 | TACATTTGTGCTCTGGAAGA | 1162 | UCUUCCAGAGCACAAAUGUA |
| g267 | CTTTACCAATGCATTTTTGT | 1128 | ACAAAAATGCATTGGTAAAG | 1163 | CUUUACCAAUGCAUUUUUGU |
| g268 | GGGCTTTACCAATGCATTTT | 1129 | AAAATGCATTGGTAAAGCCC | 1164 | GGGCUUUACCAAUGCAUUUU |
| g278 | GTAGGAATTTATAAAATCCC | 1130 | GGGATTTTATAAATTCCTAC | 1165 | GUAGGAAUUUAUAAAAUCCC |
| g316 | CACTTACCAGGCAGAGGAGT | 1131 | ACTCCTCTGCCTGGTAAGTG | 1166 | CACUUACCAGGCAGAGGAGU |
| g321 | GCCTGGTAAGTGTCTATTCT | 1132 | AGAATAGACACTTACCAGGC | 1167 | GCCUGGUAAGUGUCUAUUCU |
| g324 | GGTAAGTGTCTATTCTTGTT | 1133 | AACAAGAATAGACACTTACC | 1168 | GGUAAGUGUCUAUUCUUGUU |
| g337 | TCTTTCAGGGTTATAAGAAA | 1134 | TTTCTTATAACCCTGAAAGA | 1169 | UCUUUCAGGGUUAUAAGAAA |

TABLE 17
Features of exemplary CLL-1 gRNA

| | Spacer | | | | | | | | | | |
|---|---|---|---|---|---|---|---|---|---|---|---|
| | Sequence | | | | | | | | | | |
| | (i.e., | | | | | | | | | | |
| | Target | | | | | | | | | | |
| | Domain | | | | | | Edit- | | | | |
| | Sequence) | | | | | | able | | | | |
| | SEQ ID | PAM_ | PAM_ | dbSNP_ | OT_ | BE | Allele | BE | | Amino | Con- |
| Guide | NO. | Seq | Type | Count | Score | Window | Count | Sequence | Codon | acids | sequence |
| g7 | 1170 | AAG | Relaxed | 0 | 773 | TCaCCT | 1 | TCgCCT | — | — | splice_region_ |
| | | | PAM | | | | | | | | variant |
| | | | (NRG) | | | | | | | | |
| g7A | 1171 | TTTG | Cpf1 | 0 | 386 | aaGaTT | 3 | agGaTT | — | — | splice_region_ |
| | | | TTTN | | | | | ggGaTT | | | variant |
| | | | PAM | | | | | ggGgTT | | | |
| g8 | 1172 | CAG | Relaxed | 0 | 441 | CaCTCa | 2 | CgCTCa | — | — | splice_region_ |
| | | | PAM | | | | | CgCTCg | | | variant |
| | | | (NRG) | | | | | | | | |
| g10 | 1173 | AG | Relaxed | 0 | 1652 | CaCCTa | 2 | CgCCTa | — | — | splice_region_ |
| | | | PAM (NG) | | | | | CgCCTg | | | variant |
| g11 | 1174 | AG | Relaxed | 0 | 998 | TCTGCa | 1 | TCTGCg | ATG/ | M→V | start_lost |
| | | | PAM (NG) | | | | | | GTG | | |
| g12 | 1175 | TG | Relaxed | 0 | 1738 | TGCaTG | 1 | TGCgTG | ATG/ | M→V | start_lost |
| | | | PAM (NG) | | | | | | GTG | | |
| g13 | 1176 | AG | Relaxed | 0 | 685 | aCTCaC | 2 | gCTCaC | — | — | splice_region_ |
| | | | PAM (NG) | | | | | gCTCgC | | | variant |
| g15 | 1177 | TGG | SpCas9 | 0 | 405 | aGGTaa | 3 | gGGTaa | AAA/ | K- | splice_region_ |
| | | | NGG PAM | | | | | gGGTga | AAG- | | variant |
| | | | | | | | | gGGTgg | | | |
| g22 | 1178 | TTTC | Cpf1 | 0 | 46 | CaGCaC | 2 | CgGCaC | — | — | splice_region_ |
| | | | TTTN | | | | | CgGCgC | | | variant |
| | | | PAM | | | | | | | | |
| g29 | 1179 | TTTT | Cpf1 | 1 | 25 | TGTGGa | 1 | TGTGGg | — | — | splice_region_ |
| | | | TTTN | | | | | | | | variant |
| | | | PAM | | | | | | | | |
| g30 | 1180 | TTTA | Cpf1 | 1 | 15 | GTGGaG | 1 | GTGGgG | — | — | splice_region_ |
| | | | TTTN | | | | | | | | variant |
| | | | PAM | | | | | | | | |
| g38 | 1181 | TGG | SpCas9 | 0 | 210 | GGTaaT | 2 | GGTgaT | — | — | splice_region_ |
| | | | NGG PAM | | | | | GGTggT | | | variant |
| g59 | 1182 | TTTC | Cpf1 | 0 | 54 | GTaGGa | 2 | GTgGGa | — | — | splice_region_ |
| | | | TTTN | | | | | GTgGGg | | | variant |
| | | | PAM | | | | | | | | |
| g60 | 1183 | TG | Relaxed | 0 | 1022 | TaCCTT | 1 | TgCCTT | — | — | splice_region_ |
| | | | PAM (NG) | | | | | | | | variant |
| g64 | 1184 | GG | Relaxed | 0 | 1043 | GGTaaG | 2 | GGTgaG | — | — | splice_region_ |
| | | | PAM (NG) | | | | | GGTggG | | | variant |
| g69 | 1185 | AAG | Relaxed | 0 | 262 | TaCaTa | 3 | TgCaTa | — | — | splice_region_ |
| | | | PAM | | | | | TgCgTa | | | variant |
| | | | (NRG) | | | | | TgCgTg | | | |
| g69 | 1186 | TTTC | Cpf1 | 0 | 70 | TTTCaG | 1 | TTTCgG | — | — | splice_region_ |
| | | | TTTN | | | | | | | | variant |
| | | | PAM | | | | | | | | |
| g70 | 1187 | AAG | Relaxed | 1 | 122 | GGaGTT | 1 | GGgGTT | — | — | splice_region_ |
| | | | PAM | | | | | | | | variant |
| | | | (NRG) | | | | | | | | |
| g104 | 1188 | TAG | Relaxed | 0 | 547 | aCCTTG | 1 | gCCTTG | — | — | splice_region_ |
| | | | PAM | | | | | | | | variant |
| | | | (NRG) | | | | | | | | |
| g106 | 1189 | GAG | Relaxed | 0 | 673 | TaaTCT | 2 | TgaTCT | — | — | splice_region_ |
| | | | PAM | | | | | TggTCT | | | variant |
| | | | (NRG) | | | | | | | | |
| g110 | 1190 | AAG | Relaxed | 0 | 551 | CTTCCa | 1 | CTTCCg | — | — | splice_region_ |
| | | | PAM | | | | | | | | variant |
| | | | (NRG) | | | | | | | | |
| g120 | 1191 | AG | Relaxed | 0 | 560 | aCaTaC | 3 | gCaTaC | — | — | splice_region_ |
| | | | PAM (NG) | | | | | gCgTaC | | | variant |
| | | | | | | | | gCgTgC | | | |
| g127 | 1192 | TG | Relaxed | 1 | 212 | GTGGaG | 1 | GTGGgG | — | — | splice_region_ |
| | | | PAM (NG) | | | | | | | | variant |
| g128 | 1193 | AG | Relaxed | 1 | 199 | GaGTTC | 1 | GgGTTC | — | — | splice_region_ |
| | | | PAM (NG) | | | | | | | | variant |
| g154 | 1194 | CAG | Relaxed | 0 | 484 | TaGGaa | 3 | TgGGaa | — | — | splice_region_ |
| | | | PAM | | | | | TgGGga | | | variant |
| | | | (NRG) | | | | | TgGGgg | | | |
| g183 | 1195 | TAG | Relaxed | 0 | 408 | GTaaGT | 2 | GTgaGT | — | — | splice_region_ |
| | | | PAM | | | | | GTggGT | | | variant |
| | | | (NRG) | | | | | | | | |
| g197 | 1196 | GG | Relaxed | 0 | 14551 | GTaaTC | 2 | GTgaTC | — | — | splice_region_ |
| | | | PAM (NG) | | | | | GTggTC | | | variant |
| g202 | 1197 | AG | Relaxed | 0 | 866 | TTCCaG | 1 | TTCCgG | — | — | splice_region_ |
| | | | PAM (NG) | | | | | | | | variant |
| g267 | 1198 | TG | Relaxed | 0 | 956 | TTaCCa | 2 | TTgCCa | — | — | splice_region_ |
| | | | PAM (NG) | | | | | TTgCCg | | | variant |
| g268 | 1199 | TG | Relaxed | 0 | 462 | GCTTTa | 1 | GCTTTg | — | — | splice_region_ |
| | | | PAM (NG) | | | | | | | | variant |
| g278 | 1200 | AG | Relaxed | 0 | 530 | aGGaaT | 3 | gGGaaT | — | — | splice_region_ |
| | | | PAM (NG) | | | | | gGGgaT | | | variant |
| | | | | | | | | gGGggT | | | |
| g316 | 1201 | TG | Relaxed | 0 | 716 | CTTaCC | 1 | CTTgCC | — | — | splice_region_ |
| | | | PAM (NG) | | | | | | | | variant |
| g321 | 1202 | TG | Relaxed | 0 | 316 | CTGGTa | 1 | CTGGTg | — | — | splice_region_ |
| | | | PAM (NG) | | | | | | | | variant |
| g324 | 1203 | AG | Relaxed | 0 | 476 | TaaGTG | 2 | TgaGTG | — | — | splice_region_ |
| | | | PAM (NG) | | | | | TggGTG | | | variant |
| g337 | 1204 | CG | Relaxed | 0 | 1219 | TTTCaG | 1 | TTTCgG | — | — | splice_region_ |
| | | | PAM (NG) | | | | | | | | variant |

TABLE 8
Exemplary target domain sequences of human CLL-1 bound by various gRNAs
are provided herein. For each target domain, a DNA target sequence in
the human CLL-1 genomic sequence is provided. A gRNA targeting a
target domain provided herein may comprise an equivalent RNA sequence
within its targeting domain.

| Identifier | Target Gene | Target Domain Sequence | SEQ ID NO. |
|---|---|---|---|
| CLL-1_CBEsg3 | CLL-1 | CAAATCCATCTCCTTGGACA | 12 |
| CLL-1_CBEsg4 | CLL-1 | TTATGCCAAATCCATCTCCT | 13 |
| CLL-1_CBEsg1 | CLL-1 | ATTCCAGAACTCCAGTGAGA | 14 |
| CLL-1_CBEsg2 | CLL-1 | CGCCATACATGAGAGGGAGC | 15 |
| CLL-1_CBEsg5 | CLL-1 | AACATGGCAGGAGAGTAAAA | 16 |

TABLE 9
Exemplary target domains of human CLL-1 bound by various gRNAs
are described herein. For each target domain, the first
sequence represents a 20-nucleotide DNA sequence corresponding
to the target domain sequence that can be targeted by a suitable
gRNA, which may comprise an equivalent RNA targeting domain
sequence (comprising RNA nucleotides instead of the DNA
nucleotides in the sequences provided below), and the second
sequence is the reverse complement thereof. Bolding indicates
that the sequence is present in the human CLL-1 cDNA sequence
shown below as SEQ ID NO: 600.

| | Target Domain Sequences |
|---|---|
| gRNA A | CCCAGAAATTGGCAAATTTG (SEQ ID NO: 301) |
| | CAAATTTGCCAATTTCTGGG (SEQ ID NO: 311) |
| gRNA B | ATTCCAGAACTCCAGTGAGA (SEQ ID NO: 302) |
| | TCTCACTGGAGTTCTGGAAT (SEQ ID NO: 312) |
| gRNA C | GAGCTATATAGCAAAGAACA (SEQ ID NO: 303) |
| | TGTTCTTTGCTATATAGCTC (SEQ ID NO: 313) |
| gRNA D | GGTGGCTATTGTTTGCAGTG (SEQ ID NO: 304) |
| | CACTGCAAACAATAGCCACC (SEQ ID NO: 314) |
| gRNA E | TTGTTTGCAGTGTGGTGGAG (SEQ ID NO: 305) |
| | CTCCACCACACTGCAAACAA (SEQ ID NO: 315) |
| gRNA F | TAGCTCACGACATAATTTGG (SEQ ID NO: 306) |
| | CCAAATTATGTCGTGAGCTA (SEQ ID NO: 316) |
| gRNA G | GGCTATTGTTTGCAGTGTGG (SEQ ID NO: 307) |
| | CCACACTGCAAACAATAGCC (SEQ ID NO: 317) |
| gRNA H | GGAGAGGTTCCTGATCTTGT (SEQ ID NO: 308) |
| | ACAAGATCAGGAACCTCTCC (SEQ ID NO: 318) |
| gRNA I | TGAATATCTCCAACAAGATC (SEQ ID NO: 309) |
| | GATCTTGTTGGAGATATTCA (SEQ ID NO: 319) |
| gRNA J | GTTGTAGAGAAATATTTCTC (SEQ ID NO: 310) |
| | GAGAAATATTTCTCTACAAC (SEQ ID NO: 320) |
| gRNA O2 | ATATAATCAACTCCTCTGCC (SEQ ID NO: 340) |
| | GGCAGAGGAGTTGATTATAT (SEQ ID NO: 341) |
| gRNA P2 | AATAGACACTTACCAGGCAG (SEQ ID NO: 342) |
| | CTGCCTGGTAAGTGTCTATT (SEQ ID NO: 343) |

TABLE 10
Exemplary target domain sequences of human CLL-1 bound by various gRNAs
are provided herein. For each target domain, the first sequence
represents a DNA target sequence adjacent to a suitable PAM in the
human CLL-1 genomic sequence, and the second sequence represents an
exemplary suitable gRNA targeting domain sequence.

| | Sequences | PAM |
|---|---|---|
| gRNA A | CCCAGAAATTGGCAAATTTG (SEQ ID NO: 301) | GGG |
| | CCCAGAAAUUGGCAAAUUUG (SEQ ID NO: 321) | |
| gRNA B | ATTCCAGAACTCCAGTGAGA (SEQ ID NO: 302) | TGG |
| | AUUCCAGAACUCCAGUGAGA (SEQ ID NO: 322) | |
| gRNA C | GAGCTATATAGCAAAGAACA (SEQ ID NO: 303) | AGG |
| | GAGCUAUAUAGCAAAGAACA (SEQ ID NO: 323) | |
| gRNA D | GGTGGCTATTGTTTGCAGTG (SEQ ID NO: 304) | TGG |
| | GGUGGCUAUUGUUUGCAGUG (SEQ ID NO: 324) | |
| gRNA E | TTGTTTGCAGTGTGGTGGAG (SEQ ID NO: 305) | AGG |
| | UUGUUUGCAGUGUGGUGGAG (SEQ ID NO: 325) | |
| gRNA F | TAGCTCACGACATAATTTGG (SEQ ID NO: 306) | TGG |
| | UAGCUCACGACAUAAUUUGG (SEQ ID NO: 326) | |
| gRNA G | GGCTATTGTTTGCAGTGTGG (SEQ ID NO: 307) | TGG |
| | GGCUAUUGUUUGCAGUGUGG (SEQ ID NO: 327) | |
| gRNA H | GGAGAGGTTCCTGATCTTGT (SEQ ID NO: 308) | TGG |
| | GGAGAGGUUCCUGAUCUUGU (SEQ ID NO: 328) | |
| gRNA I | TGAATATCTCCAACAAGATC (SEQ ID NO: 309) | AGG |
| | UGAAUAUCUCCAACAAGAUC (SEQ ID NO: 329) | |
| gRNA J | GTTGTAGAGAAATATTTCTC (SEQ ID NO: 310) | TGG |
| | GUUGUAGAGAAAUAUUUCUC (SEQ ID NO: 330) | |
| gRNA O2 | ATATAATCAACTCCTCTGCC (SEQ ID NO: 340) | TGG |
| | AUAUAAUCAACUCCUCUGCC (SEQ ID NO: 344) | |
| gRNA P2 | AATAGACACTTACCAGGCAG (SEQ ID NO: 342) | AGG |
| | AAUAGACACUUACCAGGCAG (SEQ ID NO: 345) | |

TABLE 11
Exemplary target domain sequences of human CLL-1 bound by
various gRNAs are provided herein. For each target domain, a
DNA target sequence adjacent to a suitable PAM in the human
CLL-1 genomic sequence is provided. A gRNA targeting a target
domain provided herein may comprise an equivalent RNA sequence
within its targeting domain.

| | SEQ ID NO: | Sequence | PAM |
|---|---|---|---|
| gRNA K | 400 | ATGCAGAAGTCAAATTTGAA | AGG |
| gRNA L | 401 | TCAAATTTGACTTCTGCATG | TGG |
| gRNA M | 402 | TTTGACTTCTGCATGTGGAT | AGG |
| gRNA N | 403 | GGATAGGTGAGTGTCTTTTG | TGG |
| gRNA O | 404 | TTTCCATCTCACTGGAGTTC | TGG |
| gRNA P | 405 | TGGGATTTTTTCCATCTCAC | TGG |
| gRNA Q | 406 | ATGGAAAAAATCCCAGAAAT | TGG |
| gRNA R | 407 | ATCCCAGAAATTGGCAAATT | TGG |
| gRNA S | 408 | TCCCAGAAATTGGCAAATTT | GGG |
| gRNA T | 409 | CCCCAAATTTGCCAATTTCT | GGG |
| gRNA U | 410 | TCCCCAAATTTGCCAATTTC | TGG |
| gRNA V | 411 | ATTGGCAAATTTGGGGAAAA | AGG |
| gRNA W | 412 | AAAGGTAAGATTTTGAGTTA | TGG |
| gRNA X | 413 | AGAGGGAGCTGGAGGTGCTG | TGG |
| gRNA Y | 414 | CTCCAGCTCCCTCTCATGTA | TGG |
| gRNA Z | 415 | CATACATGAGAGGGAGCTGG | AGG |
| gRNA A1 | 416 | CGCCATACATGAGAGGGAGC | TGG |
| gRNA B1 | 417 | GCTGGACGCCATACATGAGA | GGG |
| gRNA C1 | 418 | TGCTGGACGCCATACATGAG | AGG |
| gRNA D1 | 419 | AGAGTCAGAAACAAGGCTGC | TGG |
| gRNA E1 | 420 | GCACAGAAGAGTCAGAAACA | AGG |
| gRNA F1 | 421 | CTGTGCCTTCTGTTGCTCAT | TGG |
| gRNA G1 | 422 | CCTTCTGTTGCTCATTGGAT | TGG |
| gRNA H1 | 423 | CTTCTGTTGCTCATTGGATT | GGG |
| gRNA I1 | 424 | CCAATCCAATGAGCAACAGA | AGG |
| gRNA J1 | 425 | GCTCATTGGATTGGGAGTCT | TGG |
| gRNA K1 | 426 | ATATAGCTCACGACATAATT | TGG |
| gRNA L1 | 427 | AAGGTAATCTTGTATTCTCT | TGG |
| gRNA M1 | 428 | CAAGGCTTACATTTGTGCTC | TGG |
| gRNA N1 | 429 | ACAAATGTAAGCCTTGTCCA | AGG |
| gRNA O1 | 430 | GTAAGCCTTGTCCAAGGAGA | TGG |
| gRNA P1 | 431 | CTTGTCCAAGGAGATGGATT | TGG |
| gRNA Q1 | 432 | CAAATCCATCTCCTTGGACA | AGG |
| gRNA R1 | 433 | AAGGAGATGGATTTGGCATA | AGG |
| gRNA S1 | 434 | TTATGCCAAATCCATCTCCT | TGG |
| gRNA T1 | 435 | TAAGTGATGATGTCCAAACA | TGG |
| gRNA U1 | 436 | ATGTTTGGACATCATCACTT | AGG |
| gRNA V1 | 437 | TGATGATGTCCAAACATGGC | AGG |
| gRNA W1 | 438 | AACATGGCAGGAGAGTAAAA | TGG |
| gRNA X1 | 439 | TTTTACTCTCCTGCCATGTT | TGG |
| gRNA Y1 | 440 | GCTGGCATTCTGAGCAGCAC | AGG |
| gRNA Z1 | 441 | GTTGTTTATCTTCAACAGGC | TGG |
| gRNA A2 | 442 | TTTTGTTGTTTATCTTCAAC | AGG |
| gRNA B2 | 443 | GATAAACAACAAAAATGCAT | TGG |
| gRNA C2 | 444 | AAAATGCATTGGTAAAGCCC | AGG |
| gRNA D2 | 445 | AGACTATCTGTATTTCCTGT | AGG |
| gRNA E2 | 446 | GGGATTTTATAAATTCCTAC | AGG |
| gRNA F2 | 447 | AGAGTAGATCATATGACTAT | TGG |
| gRNA G2 | 448 | ATAGTCATATGATCTACTCT | GGG |
| gRNA H2 | 449 | AATAGTCATATGATCTACTC | TGG |
| gRNA I2 | 450 | TAGATCATATGACTATTGGC | TGG |
| gRNA J2 | 451 | AGATCATATGACTATTGGCT | GGG |
| gRNA K2 | 452 | CCTGAAGAAGATTCCACTCG | TGG |
| gRNA L2 | 453 | CCACGAGTGGAATCTTCTTC | AGG |
| gRNA M2 | 454 | TTCCACTCGTGGTATGAGAG | TGG |
| gRNA N2 | 455 | ATCCACTCTCATACCACGAG | TGG |
| gRNA Q2 | 456 | AACAAGAATAGACACTTACC | AGG |
| gRNA R2 | 457 | AATTCTTTACTTTCTCTTTC | AGG |
| gRNA S2 | 458 | ATTCTTTACTTTCTCTTTCA | GGG |
| gRNA T2 | 459 | GACTTAAATAACATGTATTG | TGG |
| gRNA U2 | 460 | CAATACATGTTATTTAAGTC | AGG |
| gRNA V2 | 461 | AAGAATGATATGTGAGAAGA | TGG |
| gRNA W2 | 462 | ATGGCCAATCCAGTGCAGCT | TGG |
| gRNA X2 | 463 | AGAACCAAGCTGCACTGGAT | TGG |
| gRNA Y2 | 464 | TATGTAGAACCAAGCTGCAC | TGG |
| gRNA Z2 | 465 | AGCTTGGTTCTACATATTTT | AGG |
| gRNA A3 | 466 | GCTTGGTTCTACATATTTTA | GGG |
| gRNA B3 | 467 | TGGTTCTACATATTTTAGGG | AGG |
| gRNA C3 | 468 | ACATATTTTAGGGAGGCATG | AGG |
| gRNA D3 | 469 | TATTTTGAAGATCAGAGATG | TGG |
| gRNA E3 | 470 | ATCTTCAAAATATCATGATT | TGG |
| gRNA F3 | 471 | TCTTCAAAATATCATGATTT | GGG |
| gRNA G3 | 472 | TATCATGATTTGGGTTTTCC | TGG |
| gRNA H3 | 473 | TCTCTTCTTGTTTTACTTCC | AGG |
| gRNA I3 | 474 | CTGTCCACCTCTTTGCATTA | AGG |
| gRNA J3 | 475 | CITAATGCAAAGAGGTGGAC | AGG |
| gRNA K3 | 476 | CCTCTTTGCATTAAGGTAGT | TGG |
| gRNA L3 | 477 | ACTACCTTAATGCAAAGAGG | TGG |
| gRNA M3 | 478 | CCAACTACCTTAATGCAAAG | AGG |
| gRNA N3 | 479 | TTAAGGTAGTTGGTCCACCT | TGG |
| gRNA O3 | 480 | GAAGGCTGGCATGACCAAGG | TGG |
| gRNA P3 | 481 | GAGGAAGGCTGGCATGACCA | AGG |
| gRNA Q3 | 482 | TTAATGTGTGAGAGGAAGGC | TGG |
| gRNA R3 | 483 | TTATTTAATGTGTGAGAGGA | AGG |
| gRNA S3 | 484 | AACATTATTTAATGTGTGAG | AGG |
| gRNA T3 | 485 | ATAATGTTTGTTCTCACAGA | AGG |
| gRNA U3 | 486 | TGTTTGTTCTCACAGAAGGT | AGG |
| gRNA V3 | 487 | GTTTGTTCTCACAGAAGGTA | GGG |
| gRNA W3 | 488 | ATTGTGCTTATTTTCATAAT | GGG |
| gRNA X3 | 489 | AATTGTGCTTATTTTCATAA | TGG |
| gRNA Y3 | 490 | GAGTTTTTGGAAGATGGAAA | AGG |
| gRNA Z3 | 491 | AGGTTTGAGTTTTTGGAAGA | TGG |
| gRNA A4 | 492 | CCAAAAACTCAAACCTGTGA | AGG |
| gRNA B4 | 493 | CAAAAACTCAAACCTGTGAA | GGG |
| gRNA C4 | 494 | CCTTCACAGGTTTGAGTTTT | TGG |
| gRNA D4 | 495 | TATTCTAACTTTCCCTTCAC | AGG |
| gRNA E4 | 496 | TCAGAAAATATGTAAGTCAC | TGG |
| gRNA F4 | 497 | CTGGAAGTGGGAGGATGGCT | CGG |
| gRNA G4 | 498 | GAGGTCTGGAAGTGGGAGGA | TGG |
| gRNA H4 | 499 | GAATGAGGTCTGGAAGTGGG | AGG |
| gRNA I4 | 500 | GTCGAATGAGGTCTGGAAGT | GGG |
| gRNA J4 | 501 | AGTCGAATGAGGTCTGGAAG | TGG |
| gRNA K4 | 502 | TCCAGACCTCATTCGACTTC | TGG |
| gRNA L4 | 503 | GCCAGAAGTCGAATGAGGTC | TGG |
| gRNA M4 | 504 | ATCTCGCCAGAAGTCGAATG | AGG |
| gRNA N4 | 505 | ACTCATTTAATTCGTTGGGT | CGG |
| gRNA 04 | 506 | CCAACGAATTAAATGAGTCC | TGG |
| gRNA P4 | 507 | CAGGACTCATTTAATTCGTT | GGG |
| gRNA Q4 | 508 | CCAGGACTCATTTAATTCGT | TGG |
| gRNA R4 | 509 | AATAGGAGTACATCAAAGCC | AGG |
| gRNA S4 | 510 | TGTTTATTTATGTTTACAAT | AGG |

A representative CLL-1 (NM_138337.6) cDNA sequence is provided below as SEQ ID NO: 31. Underlining, holding, or italics indicates the regions complementary to gRNA A, B, C, D, E, F, G, H, I, J, or 02 (or the reverse complement thereof). Bolding and italics are used where there is overlap between two or more such regions.

(SEQ ID NO: 600)
GGCTCATTTGCAGACATATGGGTGATTGGTACAGTAGGTTTATAAACAGAAGTTTAAACTTGTA
AGCTTAAGCTTCCGTTTATAAACAGAAGTTTAAAATTATAGGTCCTGTTTAACATTCAGCTCTG
TTAACTCACTCATCTTTTTGTGTTTTTACACTTTGTCAAGATTTCTTTACATATTCATCAATGT
CTGAAGAAGTTACTTATGCAGATCTTCAATTCCAGAACTCCAGTGAGATGGAAAAAATCCCAGA
AATTGGCAAATTTGGGGAAAAAGCACCTCCAGCTCCCTCTCATGTATGGCGTCCAGCAGCCTTG
TTTCTGACTCTTCTGTGCCTTCTGTTGCTCATTGGATTGGGAGTCTTGGCAAGCATGTTTCACG
TAACTTTGAAGATAGAAATGAAAAAAATGAACAAACTACAAAACATCAGTGAAGAGCTCCAGAG
AAATATTTCTCTACAACTGATGAGTAACATGAATATCTCCAACAAGATCAGGAACCTCTCCACC
ACACTGCAAACAATAGCCACCAAATTATGTCGTGAGCTATATAGCAAAGAACAAGAGCACAAAT
GTAAGCCTTGTCCAAGGAGATGGATTTGGCATAAGGACAGCTGTTATTTCCTAAGTGATGATGT
CCAAACATGGCAGGAGAGTAAAATGGCCTGTGCTGCTCAGAATGCCAGCCTGTTGAAGATAAAC
AACAAAAATGCATTGGAATTTATAAAATCCCAGAGTAGATCATATGACTATTGGCTGGGATTAT
CTCCTGAAGAAGATTCCACTCGTGGTATGAGAGTGGATAATATAATCAACTCCTCTGCCTGGGT
TATAAGAAACGCACCTGACTTAAATAACATGTATTGTGGATATATAAATAGACTATATGTTCAA
TATTATCACTGCACTTATAAAAAAAGAATGATATGTGAGAAGATGGCCAATCCAGTGCAGCTTG
GTTCTACATATTTTAGGGAGGCATGAGGCATCAATCAAATACATTTAAGGAGTGTAGGGGGTGG
GGGTTCTAGGCTATAGGTAAATTTAAATATTTTCTGGTTGACAATTAGTTGAGTTTGTCTGAAG
ACCTGGGATTTTATCATGCAGATGAAACATCCAGGTAGCAAGCTTCAGAGAGAATAGACTGTGA
ATGTTAATGCCAGAGAGGTATAATGAAGCATGTCCCACCTCCCACTTTCCATCATGGCCTGAAC
CCTGGAGGAAGAGGAAGTCCATTCAGATAGTTGTGGGGGGCCTTCGAATTTTCATTTTCATTTA
CGTTCTTCCCCTTCTGGCCAAGATTTGCCAGAGGCAACATCAAAAACCAGCAAATTTTAATTTT
GTCCCACAGCGTTGCTAGGGTGGCATGGCTCCCCATCTCGGGTCCATCCTATACTTCCATGGGA
CTCCCTATGGCTGAAGGCCTTATGAGTCAAAGGACTTATAGCCAATTGATTGTTCTAGGCCAGG
TAAGAATGGATATGGACATGCATTTATTACCTCTTAAAATTATTATTTTAAGTAAAAGCCAATA
AACAAAAACGAAAAGGCAA
An additional CLL-1 isoform (ENST00000355690.8) cDNA is provided as:
(SEQ ID NO: 601)
GGAAGAACAGCCTTTCAAATTTGACTTCTGCATGTGGATAGATTTCTTTACATATTCATCAATG
TCTGAAGAAGTTACTTATGCAGATCTTCAATTCCAGAACTCCAGTGAGATGGAAAAAATCCCAG
AAATTGGCAAATTTGGGGAAAAAGCACCTCCAGCTCCCTCTCATGTATGGCGTCCAGCAGCCTT
GTTTCTGACTCTTCTGTGCCTTCTGTTGCTCATTGGATTGGGAGTCTTGGCAAGCATGTTTCAC
GTAACTTTGAAGATAGAAATGAAAAAAATGAACAAACTACAAAACATCAGTGAAGAGCTCCAGA
GAAATATTTCTCTACAACTGATGAGTAACATGAATATCTCCAACAAGATCAGGAACCTCTCCAC
CACACTGCAAACAATAGCCACCAAATTATGTCGTGAGCTATATAGCAAAGAACAAGAGCACAAA
TGTAAGCCTTGTCCAAGGAGATGGATTTGGCATAAGGACAGCTGTTATTTCCTAAGTGATGATG
TCCAAACATGGCAGGAGAGTAAAATGGCCTGTGCTGCTCAGAATGCCAGCCTGTTGAAGATAAA
CAACAAAAATGCATTGGAATTTATAAAATCCCAGAGTAGATCATATGACTATTGGCTGGGATTA
TCTCCTGAAGAAGATTCCACTCGTGGTATGAGAGTGGATAATATAATCAACTCCTCTGCCTGGG
TTATAAGAAACGCACCTGACTTAAATAACATGTATTGTGGATATATAAATAGACTATATGTTCA
ATATTATCACTGCACTTATAAAAAAAGAATGATATGTGAGAAGATGGCCAATCCAGTGCAGCTT
GGTTCTACATATTTTAGGGAGGCATGAGGCATCAATCAAATACATTTAAGGAGTGTAGGGGGTG
GGGGTTCTAGGCTATAGGTAAATTTAAATATTTTCTGGTTGACAATTAGTTGAGTTTGTCTGAA
GACCTGGGATTTTATCATGCAGATGAAACATCCAGGTAGCAAGCTTCAGAGAGAATAGACTGTG
AATGTTAATGCCAGAGAGGTATAATGAAGCATGTCCCACCTCCCACTTTCCATCATGGCCTGAA
CCCTGGAGGAAGAGGAAGTCCATTCAGATAGTTGTGGGGGGCCTTCGAATTTTCATTTTCATTT
ACGTTCTTCCCCTTCTGGCCAAGATTTGCCAGAGGCAACATCAAAAACCAGCAAATTTTAATTT
TGTCCCACAGCGTTGCTAGGGTGGCATGGCTCCCCATCTCGGGTCCATCCTATACTTCCATGGG
ACTCCCTATGGCTGAAGGCCTTATGAGTCAAAGGACTTATAGCCAATTGATTGTTCTAGGCCAG
GTAAGAATGGATATGGACATGCATTTATTACCTCTTAAAATTATTATTTTAAGTAAAAGCCAAT
AAACAAAAACGAAAAGGCAA
An additional CLL-1 isoform (NM_001207010.2) cDNA is provided as:
(SEQ ID NO: 602)
CTATTTAGCATTGCTGCTGCCAGCCCCAACCACATTTCTGATTGCCTAGGAAGAACAGCCTTTC
AAATTTGACTTCTGCATGTGGATAGATTTCTTTACATATTCATCAATGTCTGAAGAAGTTACTT
ATGCAGATCTTCAATTCCAGAACTCCAGTGAGATGGAAAAAATCCCAGAAATTGGCAAATTTGG
GGAAAAAGCACCTCCAGCTCCCTCTCATGTATGGCGTCCAGCAGCCTTGTTTCTGACTCTTCTG
TGCCTTCTGTTGCTCATTGGATTGGGAGTCTTGGCAAGCATGTTTCACGTAACTTTGAAGATAG
AAATGAAAAAAATGAACAAACTACAAAACATCAGTGAAGAGCTCCAGAGAAATATTTCTCTACA
ACTGATGAGTAACATGAATATCTCCAACAAGATCAGGAACCTCTCCACCACACTGCAAACAATA
GCCACCAAATTATGTCGTGAGCTATATAGCAAAGAACAAGAGCACAAATGTAAGCCTTGTCCAA
GGAGATGGATTTGGCATAAGGACAGCTGTTATTTCCTAAGTGATGATGTCCAAACATGGCAGGA
GAGTAAAATGGCCTGTGCTGCTCAGAATGCCAGCCTGTTGAAGATAAACAACAAAAATGCATTG
GAATTTATAAAATCCCAGAGTAGATCATATGACTATTGGCTGGGATTATCTCCTGAAGAAGATT
CCACTCGTGGTATGAGAGTGGATAATATAATCAACTCCTCTGCCTGGGTTATAAGAAACGCACC
TGACTTAAATAACATGTATTGTGGATATATAAATAGACTATATGTTCAATATTATCACTGCACT
TATAAAAAAAGAATGATATGTGAGAAGATGGCCAATCCAGTGCAGCTTGGTTCTACATATTTTA
GGGAGGCATGAGGCATCAATCAAATACATTTAAGGAGTGTAGGGGGTGGGGGTTCTAGGCTATA
GGTAAATTTAAATATTTTCTGGTTGACAATTAGTTGAGTTTGTCTGAAGACCTGGGATTTTATC
ATGCAGATGAAACATCCAGGTAGCAAGCTTCAGAGAGAATAGACTGTGAATGTTAATGCCAGAG
AGGTATAATGAAGCATGTCCCACCTCCCACTTTCCATCATGGCCTGAACCCTGGAGGAAGAGGA
AGTCCATTCAGATAGTTGTGGGGGGCCTTCGAATTTTCATTTTCATTTACGTTCTTCCCCTTCT
GGCCAAGATTTGCCAGAGGCAACATCAAAAACCAGCAAATTTTAATTTTGTCCCACAGCGTTGC
TAGGGTGGCATGGCTCCCCATCTCGGGTCCATCCTATACTTCCATGGGACTCCCTATGGCTGAA
GGCCTTATGAGTCAAAGGACTTATAGCCAATTGATTGTTCTAGGCCAGGTAAGAATGGATATGG
ACATGCATTTATTACCTCTTAAAATTATTATTTTAAGTAAAAGCCAATAAACAAAAACGAAAAG
GCAA
An additional CLL-1 isoform (NM_001300730.2) cDNA is provided as:
(SEQ ID NO: 603)
GGCTCATTTGCAGACATATGGGTGATTGGTACAGTAGGTTTATAAACAGAAGTTTAAACTTGTA
AGCTTAAGCTTCCGTTTATAAACAGAAGTTTAAAATTATAGGTCCTGTTTAACATTCAGCTCTG
TTAACTCACTCATCTTTTTGTGTTTTTACACTTTGTCAAGATTTCTTTACATATTCATCAATGT
CTGAAGAAGTTACTTATGCAGATCTTCAATTCCAGAACTCCAGTGAGATGGAAAAAATCCCAGA
AATTGGCAAATTTGGGGAAAAAGCACCTCCAGCTCCCTCTCATGTATGGCGTCCAGCAGCCTTG
TTTCTGACTCTTCTGTGCCTTCTGTTGCTCATTGGATTGGGAGTCTTGGCAAGCATGTTTCACG
TAACTTTGAAGATAGAAATGAAAAAAATGAACAAACTACAAAACATCAGTGAAGAGCTCCAGAG
AAATATTTCTCTACAACTGATGAGTAACATGAATATCTCCAACAAGATCAGGAACCTCTCCACC
ACACTGCAAACAATAGCCACCAAATTATGTCGTGAGCTATATAGCAAAGAACAAGAGCACAAAT
GTAAGCCTTGTCCAAGGAGATGGATTTGGCATAAGGACAGCTGTTATTTCCTAAGTGATGATGT
CCAAACATGGCAGGAGAGTAAAATGGCCTGTGCTGCTCAGAATGCCAGCCTGTTGAAGATAAAC
AACAAAAATGCATTGGAATTTATAAAATCCCAGAGTAGATCATATGACTATTGGCTGGGATTAT
CTCCTGAAGAAGATTCCACTCGTGGTATGAGAGTGGATAATATAATCAACTCCTCTGCCTGAAA
ATATCAAACGAAGAAAGAAACCAGAGTCTCAACCTGCTGGACACTATTGGAAGTCCATCATTTA
ACACGTTTTTAGTATATACTTTTAGCAGGAGACAGCTCTGAGTCAACTGTGTTGAGGTGCCACC
ACAGCGAGTTTAGGCACTCAGATCCCTGCATACTCATCACATTGGGCCATAATGGCAAATAGAA
TTTTTTGTTTTGTTTTGTTTGTTTGCTTTTTCTTTCACATAGAAATAGTAAGTGTAGGAGTGTG
GGTCAGAAAGAAAAGGTGGCCCTACCTCTGATGGTTGGCAATGATAGGATACAATGGGAGATAA
GCTATCTACAAATGGAGTGGAGAAGGATATATATTTCAAAGGCCTAATTTGTAGTGAAAGACTA
GAGACAAAGGTAATGTGTGTGTCAGGAGAGAGTACAGATGGAATCTTGTTTTGCAAACGTAGAA
TATGTATGTGTTTGTAATTATTGCAAATGGAATGGTAATCTATAATGGAATGGAAAACATTGTA
GATATTTTCAGTTATCAAAAAGAAAACTGAAAAAGTATATAATAATTGTATGTATGATATATAT
ATGTGTGTGTGTGTGTATATATATCTTCACTTTATAACTCTGTGTTGTTTTGGGGTTTGTTTCT
GAAAGGGGGTTGTAATAAATGACATCTGTACTATGTCACCACAAATAAATCTCATTCTTAAACA
TTTAATTGATGAACTTA
An additional CLL-1 isoform (NM_201623.4) cDNA is provided as:
(SEQ ID NO: 604)
GGCTCATTTGCAGACATATGGGTGATTGGTACAGTAGGTTTATAAACAGAAGTTTAAACTTGTA
AGCTTAAGCTTCCGTTTATAAACAGAAGTTTAAAATTATAGGTCCTGTTTAACATTCAGCTCTG
TTAACTCACTCATCTTTTTGTGTTTTTACACTTTGTCAAGATTTCTTTACATATTCATCAATGT
CTGAAGAAGTTACTTATGCAGATCTTCAATTCCAGAACTCCAGTGAGATGGAAAAAATCCCAGA
AATTGGCAAATTTGGGGAAAAAGTTCACGTAACTTTGAAGATAGAAATGAAAAAAATGAACAAA
CTACAAAACATCAGTGAAGAGCTCCAGAGAAATATTTCTCTACAACTGATGAGTAACATGAATA
TCTCCAACAAGATCAGGAACCTCTCCACCACACTGCAAACAATAGCCACCAAATTATGTCGTGA
GCTATATAGCAAAGAACAAGAGCACAAATGTAAGCCTTGTCCAAGGAGATGGATTTGGCATAAG
GACAGCTGTTATTTCCTAAGTGATGATGTCCAAACATGGCAGGAGAGTAAAATGGCCTGTGCTG
CTCAGAATGCCAGCCTGTTGAAGATAAACAACAAAAATGCATTGGAATTTATAAAATCCCAGAG
TAGATCATATGACTATTGGCTGGGATTATCTCCTGAAGAAGATTCCACTCGTGGTATGAGAGTG
GATAATATAATCAACTCCTCTGCCTGGGTTATAAGAAACGCACCTGACTTAAATAACATGTATT
GTGGATATATAAATAGACTATATGTTCAATATTATCACTGCACTTATAAAAAAAGAATGATATG
TGAGAAGATGGCCAATCCAGTGCAGCTTGGTTCTACATATTTTAGGGAGGCATGAGGCATCAAT
CAAATACATTTAAGGAGTGTAGGGGGTGGGGGTTCTAGGCTATAGGTAAATTTAAATATTTTCT
GGTTGACAATTAGTTGAGTTTGTCTGAAGACCTGGGATTTTATCATGCAGATGAAACATCCAGG
TAGCAAGCTTCAGAGAGAATAGACTGTGAATGTTAATGCCAGAGAGGTATAATGAAGCATGTCC
CACCTCCCACTTTCCATCATGGCCTGAACCCTGGAGGAAGAGGAAGTCCATTCAGATAGTTGTG
GGGGGCCTTCGAATTTTCATTTTCATTTACGTTCTTCCCCTTCTGGCCAAGATTTGCCAGAGGC
AACATCAAAAACCAGCAAATTTTAATTTTGTCCCACAGCGTTGCTAGGGTGGCATGGCTCCCCA
TCTCGGGTCCATCCTATACTTCCATGGGACTCCCTATGGCTGAAGGCCTTATGAGTCAAAGGAC
TTATAGCCAATTGATTGTTCTAGGCCAGGTAAGAATGGATATGGACATGCATTTATTACCTCTT
AAAATTATTATTTTAAGTAAAAGCCAATAAACAAAAACGAAAAGGCAA

gRNAs Targeting CD123

The present disclosure provides a number of useful gRNAs that can target an endonuclease to human CD123. In some embodiments, the gRNA that can target an endonuclease to human CD33 is a gRNA disclosed in any of PCT Publication Nos. WO2020/047164 and WO2021/041977, each of which is incorporated herein by reference in its entirety. Table 1 below illustrates target domains in human endogenous CD123 that can be bound by gRNAs described herein.

TABLE 12
Exemplary target domains of human CD123 bound by various gRNAs are
described herein. For each target domain, the first sequence
represents a 20-nucleotide DNA sequence corresponding to the target
domain sequence that can be targeted by a suitable gRNA, which
may comprise an equivalent RNA targeting domain sequence (comprising
RNA nucleotides instead of DNA nucleotides), and the second sequence
is the reverse complement thereof. Bolding indicates that the
sequence is present in the human CD123 cDNA sequence shown below as
SEQ ID NO: 700.

| | Target Domain Sequences |
|---|---|
| gRNA A | GCCCTGTCTCCTGCAAACGA (SEQ ID NO: 801) |
| | TCGTTTGCAGGAGACAGGGC (SEQ ID NO: 811) |
| gRNA B | TGAGCCAAAGGAGGACCATC (SEQ ID NO: 802) |
| | GATGGTCCTCCTTTGGCTCA (SEQ ID NO: 812) |
| gRNA C | TCAGGAGCAGCGTGAGCCAA (SEQ ID NO: 803) |
| | TTGGCTCACGCTGCTCCTGA (SEQ ID NO: 813) |
| gRNA D | TCCTTCGTTTGCAGGAGACA (SEQ ID NO: 804) |
| | TGTCTCCTGCAAACGAAGGA (SEQ ID NO: 814) |
| gRNA E | ATCCACGTCATGAATCCAGC (SEQ ID NO: 805) |
| | GCTGGATTCATGACGTGGAT (SEQ ID NO: 815) |
| gRNA F | CAGGTCGTACTGGACGTCCG (SEQ ID NO: 806) |
| | CGGACGTCCAGTACGACCTG (SEQ ID NO: 816) |
| gRNA G | TTTCTTGAGCTGCAGCTGGG (SEQ ID NO: 807) |
| | CCCAGCTGCAGCTCAAGAAA (SEQ ID NO: 817) |
| gRNA H | GGTCGTACTGGACGTCCGCG (SEQ ID NO: 808) |
| | CGCGGACGTCCAGTACGACC (SEQ ID NO: 818) |
| gRNA I | AGTTCCCACATCCTGGTGCG (SEQ ID NO: 809) |
| | CGCACCAGGATGTGGGAACT (SEQ ID NO: 19) |
| gRNA J | CACTACAAAACGGATGCTCA (SEQ ID NO: 810) |
| | TGAGCATCCGTTTTGTAGTG (SEQ ID NO: 820) |
| gRNA D1 | TTCATCCTTAGGTTCGTGAT (SEQ ID NO: 840) |
| | ATCACGAACCTAAGGATGAA (SEQ ID NO: 841) |
| gRNA N3 | TTGACGCCTGCTGCGGTAAG (SEQ ID NO: 842) |
| | CTTACCGCAGCAGGCGTCAA (SEQ ID NO: 843) |
| gRNA P3 | CGAGTGTCTTCACTACAAAA (SEQ ID NO: 844) |
| | TTTTGTAGTGAAGACACTCG (SEQ ID NO: 845) |
| gRNA S3 | ATGCTCAGGGAACACGTATC (SEQ ID NO: 846) |
| | GATACGTGTTCCCTGAGCAT (SEQ ID NO: 847) |

TABLE 13
Exemplary target domain sequences of human CD123 bound by various gRNAs
are provided herein. For each target domain, the first sequence
represents a DNA target sequence adjacent to a suitable PAM in the human
CD123 genomic sequence, and the second sequence represents an
exemplary equivalent gRNA targeting domain sequence.

| | Sequences | PAM |
|---|---|---|
| gRNA A | GCCCTGTCTCCTGCAAACGA (SEQ ID NO: 801) | AGG |
| | GCCCUGUCUCCUGCAAACGA (SEQ ID NO: 821) | |
| gRNA B | TGAGCCAAAGGAGGACCATC (SEQ ID NO: 802) | GGG |
| | UGAGCCAAAGGAGGACCAUC (SEQ ID NO: 822) | |
| gRNA C | TCAGGAGCAGCGTGAGCCAA (SEQ ID NO: 803) | AGG |
| | UCAGGAGCAGCGUGAGCCAA (SEQ ID NO: 823) | |
| gRNA D | TCCTTCGTTTGCAGGAGACA (SEQ ID NO: 804) | GGG |
| | UCCUUCGUUUGCAGGAGACA (SEQ ID NO: 824) | |
| gRNA E | ATCCACGTCATGAATCCAGC (SEQ ID NO: 805) | AGG |
| | GCUGGAUUCAUGACGUGGAU (SEQ ID NO: 825) | |
| gRNA F | CAGGTCGTACTGGACGTCCG (SEQ ID NO: 806) | CGG |
| | CAGGUCGUACUGGACGUCCG (SEQ ID NO: 826) | |
| gRNA G | TTTCTTGAGCTGCAGCTGGG (SEQ ID NO: 807) | CGG |
| | UUUCUUGAGCUGCAGCUGGG (SEQ ID NO: 827) | |
| gRNA H | GGTCGTACTGGACGTCCGCG (SEQ ID NO: 808) | GGG |
| | GGUCGUACUGGACGUCCGCG (SEQ ID NO: 828) | |
| gRNA I | AGTTCCCACATCCTGGTGCG (SEQ ID NO: 809) | GGG |
| | AGUUCCCACAUCCUGGUGCG (SEQ ID NO: 829) | |
| gRNA J | CACTACAAAACGGATGCTCA (SEQ ID NO: 810) | GGG |
| | UGAGCAUCCGUUUUGUAGUG (SEQ ID NO: 830) | |
| gRNA D1 | TTCATCCTTAGGTTCGTGAT (SEQ ID NO: 840) | TGG |
| | UUCAUCCUUAGGUUCGUGAU (SEQ ID NO: 848) | |
| gRNA N3 | TTGACGCCTGCTGCGGTAAG (SEQ ID NO: 842) | CGG |
| | UUGACGCCUGCUGCGGUAAG (SEQ ID NO: 849) | |
| gRNA P3 | CGAGTGTCTTCACTACAAAA (SEQ ID NO: 844) | CGG |
| | CGAGUGUCUUCACUACAAAA (SEQ ID NO: 850) | |
| gRNA S3 | ATGCTCAGGGAACACGTATC (SEQ ID NO: 846) | GGG |
| | AUGCUCAGGGAACACGUAUC (SEQ ID NO: 851) | |

TABLE 14
Exemplary target domain sequences of human CD123
bound by various gRNAs are provided herein. For
each target domain, a DNA target sequence
adjacent to a suitable PAM in the human CD123
genomic sequence is provided. A gRNA targeting a
target domain provided herein may comprise an
equivalent RNA sequence within its targetin
domain.

| | SEQ | | |
|---|---|---|---|
| | ID NO: | Sequence | PAM |
| gRNA K | 900 | TTCCGGAGCTGCGTTCCCGA | TGG |
| gRNA L | 901 | GACCATCGGGAACGCAGCTC | CGG |
| gRNA M | 902 | CGTTCCCGATGGTCCTCCTT | TGG |
| gRNA N | 903 | GTGAGCCAAAGGAGGACCAT | CGG |
| gRNA O | 904 | GGAGCAGCGTGAGCCAAAGG | AGG |
| gRNA P | 905 | GGAGACAGGGCAGGGCGATC | AGG |
| gRNA Q | 906 | CGTTTGCAGGAGACAGGGCA | GGG |
| gRNA R | 907 | TCGTTTGCAGGAGACAGGGC | AGG |
| gRNA S | 908 | TGTCTCCTGCAAACGAAGGA | AGG |
| gRNA T | 909 | TTCCTTCGTTTGCAGGAGAC | AGG |
| gRNA U | 910 | TCTTACCTTCCTTCGTTTGC | AGG |
| gRNA V | 911 | AAACGAAGGAAGGTAAGAAC | TGG |
| gRNA W | 912 | GATCTAAAACGGTGACAGGT | TGG |
| gRNA X | 913 | TTTGGATCTAAAACGGTGAC | AGG |
| gRNA Y | 914 | TGGTGGGTTTGGATCTAAAA | CGG |
| gRNA Z | 915 | AGGTTCGTGATTGGTGGGTT | TGG |
| gRNA A1 | 916 | ACCCACCAATCACGAACCTA | AGG |
| gRNA B1 | 917 | TCCTTAGGTTCGTGATTGGT | GGG |
| gRNA C1 | 918 | ATCCTTAGGTTCGTGATTGG | TGG |
| gRNA E1 | 919 | GAACCTAAGGATGAAAGCAA | AGG |
| gRNA F1 | 920 | GAGCCTTTGCTTTCATCCTT | AGG |
| gRNA G1 | 921 | CAAAGGCTCAGCAGTTGACC | TGG |
| gRNA H1 | 922 | AAAGGCTCAGCAGTTGACCT | GGG |
| gRNA I1 | 923 | CACATTTCTGTTAAGGTCCC | AGG |
| gRNA J1 | 924 | TATCGGTCACATTTCTGTTA | AGG |
| gRNA K1 | 925 | GTCTTTAACACACTCGATAT | CGG |
| gRNA L1 | 926 | AGACGCCGACTATTCTATGC | CGG |
| gRNA M1 | 927 | ATTTACCGGCATAGAATAGT | CGG |
| gRNA N1 | 928 | CAATAGAGAGTATGATTTAC | CGG |
| gRNA O1 | 929 | CATAGTCCTATGTCTCTCTT | AGG |
| gRNA P1 | 930 | TCACTGCCTAAGAGAGACAT | AGG |
| gRNA Q1 | 931 | AACAATAGCTATTGCCAGTT | TGG |
| gRNA R1 | 932 | ATAAGGAAATTGCTCCAAAC | TGG |
| gRNA S1 | 933 | GTAGTTGGTCACTTCACATA | AGG |
| gRNA T1 | 934 | GACCAACTACACCGTCCGAG | TGG |
| gRNA U1 | 935 | GGCCACTCGGACGGTGTAGT | TGG |
| gRNA V1 | 936 | TGGTGGGTTGGCCACTCGGA | CGG |
| gRNA W1 | 937 | AGAATGGTGGGTTGGCCACT | CGG |
| gRNA X1 | 938 | CCAACCCACCATTCTCCACG | TGG |
| gRNA Y1 | 939 | CCACGTGGAGAATGGTGGGT | TGG |
| gRNA Z1 | 940 | GGATCCACGTGGAGAATGGT | GGG |
| gRNA A2 | 941 | AGGATCCACGTGGAGAATGG | TGG |
| gRNA B2 | 942 | AAGAGGATCCACGTGGAGAA | TGG |
| gRNA C2 | 943 | CTCAGGGAAGAGGATCCACG | TGG |
| gRNA D2 | 944 | TCTCACTGTTCTCAGGGAAG | AGG |
| gRNA E2 | 945 | CATTTTTCTCACTGTTCTCA | GGG |
| gRNA F2 | 946 | ACATTTTTCTCACTGTTCTC | AGG |
| gRNA G2 | 947 | TCTTTCATGTTTGTGAACCC | AGG |
| gRNA H2 | 948 | TTCATGTTTGTGAACCCAGG | TGG |
| gRNA I2 | 949 | TCATGTTTGTGAACCCAGGT | GGG |
| gRNA J2 | 950 | TGAACCCAGGTGGGAAGCCT | TGG |
| gRNA K2 | 951 | GAACCCAGGTGGGAAGCCTT | GGG |
| gRNA L2 | 952 | CCAGGTGGGAAGCCTTGGGC | AGG |
| gRNA M2 | 953 | CTGCCCAAGGCTTCCCACCT | GGG |
| gRNA N2 | 954 | CCTGCCCAAGGCTTCCCACC | TGG |
| gRNA O2 | 955 | TGGGAAGCCTTGGGCAGGTG | CGG |
| gRNA P2 | 956 | AGATTCTCCGCACCTGCCCA | AGG |
| gRNA Q2 | 957 | GTGCGGAGAATCTGACCTGC | TGG |
| gRNA R2 | 958 | GACCTGCTGGATTCATGACG | TGG |
| gRNA S2 | 959 | TGGATTTCTTGAGCTGCAGC | TGG |
| gRNA T2 | 960 | GGATTTCTTGAGCTGCAGCT | GGG |
| gRNA U2 | 961 | TTGAGCTGCAGCTGGGCGGT | AGG |
| gRNA V2 | 962 | CTGCAGCTGGGCGGTAGGCC | CGG |
| gRNA W2 | 963 | TGCAGCTGGGCGGTAGGCCC | GGG |
| gRNA X2 | 964 | GCAGCTGGGCGGTAGGCCCG | GGG |
| gRNA Y2 | 965 | CAGCTGGGCGGTAGGCCCGG | GGG |
| gRNA Z2 | 966 | GGTAGGCCCGGGGGCCCCCG | CGG |
| gRNA A3 | 967 | GGACGTCCGCGGGGGCCCCC | GGG |
| gRNA B3 | 968 | TGGACGTCCGCGGGGGCCCC | CGG |
| gRNA C3 | 969 | GTCGTACTGGACGTCCGCGG | GGG |
| gRNA D3 | 970 | AGGTCGTACTGGACGTCCGC | GGG |
| gRNA E3 | 971 | CGTTCAAGTACAGGTCGTAC | TGG |
| gRNA F3 | 972 | GTACTTGAACGTTGCCAAGT | AGG |
| gRNA G3 | 973 | ACTTGGCAACGTTCAAGTAC | AGG |
| gRNA H3 | 974 | TTGCCAAGTAGGTGTGCCCG | TGG |
| gRNA I3 | 975 | TGCCAAGTAGGTGTGCCCGT | GGG |
| gRNA J3 | 976 | TGCCCACGGGCACACCTACT | TGG |
| gRNA K3 | 977 | ACCTTACCGCTTACCGCAGC | AGG |
| gRNA L3 | 978 | GCTGCGGTAAGCGGTAAGGT | TGG |
| gRNA M3 | 979 | GCCTGCTGCGGTAAGCGGTA | AGG |
| gRNA 03 | 980 | CGTACTGTTGACGCCTGCTG | CGG |
| gRNA Q3 | 981 | TCACTACAAAACGGATGCTC | AGG |
| gRNA R3 | 982 | GATGCTCAGGGAACACGTAT | CGG |
| gRNA T3 | 983 | GACATCTCTCGACTCTCCAG | CGG |
| gRNA U3 | 984 | GTGGGAACTTTGAGAACCGC | TGG |
| gRNA V3 | 985 | TTCTCAAAGTTCCCACATCC | TGG |
| gRNA W3 | 986 | AAAGTTCCCACATCCTGGTG | CGG |
| gRNA X3 | 987 | AAGTTCCCACATCCTGGTGC | GGG |
| gRNA Y3 | 988 | CCCACATCCTGGTGCGGGGC | AGG |
| gRNA Z3 | 989 | CCTGCCCCGCACCAGGATGT | GGG |
| gRNA A4 | 990 | TCCTGCCCCGCACCAGGATG | TGG |
| gRNA B4 | 991 | CTGCGCTCCTGCCCCGCACC | AGG |
| gRNA C4 | 992 | CGGGGCAGGAGCGCAGCCTT | CGG |
| gRNA D4 | 993 | ATCTGTGCAGGGGATACCGA | AGG |
| gRNA E4 | 994 | CGACAAACTTATCTGTGCAG | GGG |
| gRNA F4 | 995 | ACGACAAACTTATCTGTGCA | GGG |
| gRNA G4 | 996 | GACGACAAACTTATCTGTGC | AGG |
| gRNA H4 | 997 | TTTGTCGTCTTTTCACAGAT | TGG |
| gRNA I4 | 998 | TCACAGATTGGTGAGTAGCC | CGG |
| gRNA J4 | 999 | CACAGATTGGTGAGTAGCCC | GGG |
| gRNA K4 | 1000 | CACTTTGCAGTCATGTTGGG | TGG |
| gRNA L4 | 1001 | TTACACTTTGCAGTCATGTT | GGG |
| gRNA M4 | 1002 | ATTACACTTTGCAGTCATGT | TGG |
| gRNA N4 | 1003 | AGACACATTCCTTTATGCAC | TGG |
| gRNA O4 | 1004 | TCTCATTTTCCAGTGCATAA | AGG |
| gRNA P4 | 1005 | CTATGAGCTTCAGATACAAA | AGG |
| gRNA Q4 | 1006 | GCAGCCTGTAATCACAGAAC | AGG |
| gRNA R4 | 1007 | CTCACCTGTTCTGTGATTAC | AGG |
| gRNA S4 | 1008 | TTTATTTTCTTTCAAACCAC | AGG |
| gRNA T4 | 1009 | GAGGTTCTGTCTCTGACCTG | TGG |
| gRNA U4 | 1010 | TCCTTCCAGCTACTCAATCC | TGG |
| gRNA V4 | 1011 | AGGATTGAGTAGCTGGAAGG | AGG |
| gRNA W4 | 1012 | TCCAGGATTGAGTAGCTGGA | AGG |
| gRNA X4 | 1013 | ACGTTCCAGGATTGAGTAGC | TGG |
| gRNA Y4 | 1014 | ATTTGTACTGTGTACGTTCC | AGG |
| gRNA Z4 | 1015 | ACACAGTACAAATAAGAGCC | CGG |
| gRNA A5 | 1016 | CACAGTACAAATAAGAGCCC | GGG |
| gRNA B5 | 1017 | GAATTCATACACTCTTTCCC | GGG |
| gRNA C5 | 1018 | AGAATTCATACACTCTTTCC | CGG |
| gRNA D5 | 1019 | TGTATGAATTCTTGAGCGCC | TGG |
| gRNA E5 | 1020 | TGGAGCACCCCCCAGCGCTT | CGG |
| gRNA F5 | 1021 | GAAGCGCTGGGGGGTGCTCC | AGG |
| gRNA G5 | 1022 | CCCCCCAGCGCTTCGGTGAG | TGG |
| gRNA H5 | 1023 | CCCCCAGCGCTTCGGTGAGT | GGG |
| gRNA I5 | 1024 | CCACTCACCGAAGCGCTGGG | GGG |
| gRNA J5 | 1025 | CCCACTCACCGAAGCGCTGG | GGG |
| gRNA K5 | 1026 | GCCCACTCACCGAAGCGCTG | GGG |
| gRNA L5 | 1027 | AGCCCACTCACCGAAGCGCT | GGG |
| gRNA M5 | 1028 | CAGCCCACTCACCGAAGCGC | TGG |
| gRNA N5 | 1029 | GCTTCGGTGAGTGGGCTGTG | CGG |
| gRNA O5 | 1030 | CTTCGGTGAGTGGGCTGTGC | GGG |
| gRNA P5 | 1031 | TTCGGTGAGTGGGCTGTGCG | GGG |
| gRNA Q5 | 1032 | TCTAGGGGTAAAGGGTGAGA | GGG |
| gRNA R5 | 1033 | CTCTAGGGGTAAAGGGTGAG | AGG |
| gRNA S5 | 1034 | TTTACCCCTAGAGTGCGACC | AGG |
| gRNA T5 | 1035 | GGTCGCACTCTAGGGGTAAA | GGG |
| gRNA U5 | 1036 | TGGTCGCACTCTAGGGGTAA | AGG |
| gRNA V5 | 1037 | ACCCCTAGAGTGCGACCAGG | AGG |
| gRNA W5 | 1038 | CCTAGAGTGCGACCAGGAGG | AGG |
| gRNA X5 | 1039 | CTAGAGTGCGACCAGGAGGA | GGG |
| gRNA Y5 | 1040 | TCCTCCTGGTCGCACTCTAG | GGG |
| gRNA Z5 | 1041 | CTCCTCCTGGTCGCACTCTA | GGG |
| gRNA A6 | 1042 | CCTCCTCCTGGTCGCACTCT | AGG |
| gRNA B6 | 1043 | GTGTGTTTGCGCCCTCCTCC | TGG |
| gRNA C6 | 1044 | AGGGCGCAAACACACGTGCC | TGG |
| gRNA D6 | 1045 | GCGCAAACACACGTGCCTGG | CGG |
| gRNA E6 | 1046 | GATCAGCAGCGACGTCCGCC | AGG |
| gRNA F6 | 1047 | GACGTCGCTGCTGATCGCGC | TGG |
| gRNA G6 | 1048 | ACGTCGCTGCTGATCGCGCT | GGG |
| gRNA H6 | 1049 | CGTCGCTGCTGATCGCGCTG | GGG |
| gRNA I6 | 1050 | GATCGCGCTGGGGACGCTGC | TGG |
| gRNA J6 | 1051 | GCTGGGGACGCTGCTGGCCC | TGG |
| gRNA K6 | 1052 | GATCACGAAGACACAGACCA | GGG |
| gRNA L6 | 1053 | AGATCACGAAGACACAGACC | AGG |
| gRNA M6 | 1054 | GTGTCTTCGTGATCTGCAGA | AGG |
| gRNA N6 | 1055 | CTGCAGAAGGTGAGCCCTCG | AGG |
| gRNA O6 | 1056 | TGCAGAAGGTGAGCCCTCGA | GGG |
| gRNA P6 | 1057 | GGCCATTTCTCTTTCCTCCG | AGG |
| gRNA Q6 | 1058 | TACCTCGGAGGAAAGAGAAA | TGG |
| gRNA R6 | 1059 | TCTCTTTCCTCCGAGGTATC | TGG |
| gRNA S6 | 1060 | TGCATCACCAGATACCTCGG | AGG |
| gRNA T6 | 1061 | CTCTGCATCACCAGATACCT | CGG |
| gRNA U6 | 1062 | TCTTTCATGTGAGGGATGCG | GGG |
| gRNA V6 | 1063 | GTCTTTCATGTGAGGGATGC | GGG |
| gRNA W6 | 1064 | GGTCTTTCATGTGAGGGATG | CGG |
| gRNA X6 | 1065 | CCTCACATGAAAGACCCCAT | CGG |
| gRNA Y6 | 1066 | CGATGGGGTCTTTCATGTGA | GGG |
| gRNA Z6 | 1067 | CCGATGGGGTCTTTCATGTG | AGG |
| gRNA A7 | 1068 | TTTGGAAGCTGTCACCGATG | GGG |
| gRNA B7 | 1069 | TTTTGGAAGCTGTCACCGAT | GGG |
| gRNA C7 | 1070 | GTTTTGGAAGCTGTCACCGA | TGG |
| gRNA D7 | 1071 | CAGCTTCCAAAACGACAAGC | TGG |
| gRNA E7 | 1072 | AACATACCAGCTTGTCGTTT | TGG |
| gRNA F7 | 1073 | CTGCCTCCTCTCGTCTCTGC | AGG |
| gRNA G7 | 1074 | CCTCCTCTCGTCTCTGCAGG | TGG |
| gRNA H7 | 1075 | CCACCTGCAGAGACGAGAGG | AGG |
| gRNA I7 | 1076 | TCTCGTCTCTGCAGGTGGTC | TGG |
| gRNA J7 | 1077 | CTCGTCTCTGCAGGTGGTCT | GGG |
| gRNA K7 | 1078 | AGACCACCTGCAGAGACGAG | AGG |
| gRNA L7 | 1079 | GTCTCTGCAGGTGGTCTGGG | AGG |
| gRNA M7 | 1080 | TCTGCAGGTGGTCTGGGAGG | CGG |
| gRNA N7 | 1081 | CTGCAGGTGGTCTGGGAGGC | GGG |
| gRNA O7 | 1082 | GTCTGGGAGGCGGGCAAAGC | CGG |
| gRNA P7 | 1083 | GGAGGCGGGCAAAGCCGGCC | TGG |
| gRNA Q7 | 1084 | GGCGGGCAAAGCCGGCCTGG | AGG |
| gRNA R7 | 1085 | AGCCGGCCTGGAGGAGTGTC | TGG |
| gRNA S7 | 1086 | CACCAGACACTCCTCCAGGC | CGG |
| gRNA T7 | 1087 | CAGTCACCAGACACTCCTCC | AGG |
| gRNA U7 | 1088 | GTGTCTGGTGACTGAAGTAC | AGG |
| gRNA V7 | 1089 | TCGTGCAGAAAACTTGAGAC | TGG |
| gRNA W7 | 1090 | CGTGCAGAAAACTTGAGACT | GGG |
| gRNA X7 | 1091 | GTGCAGAAAACTTGAGACTG | GGG |
| gRNA Y7 | 1092 | AAAACTTGAGACTGGGGTTC | AGG |
| gRNA Z7 | 1093 | AAACTTGAGACTGGGGTTCA | GGG |
| gRNA A8 | 1094 | AGACTGGGGTTCAGGGCTTG | TGG |
| gRNA B8 | 1095 | GACCTGCTGGATTCATGACG | TGG |
| gRNA C8 | 1096 | GTCGTACTGGACGTCCGCGG | GGG |
| gRNA D8 | 1097 | GGTCGTACTGGACGTCCGCG | GGG |

A representative CD123 (NM_001267713.1) cDNA sequence is provided below as SEQ ID NO: 31. Underlining or holding indicates the regions complementary to gRNA A, B, C, D, E, F, G, H, I, J, P3, or S3 (or the reverse complement thereof). Bolding is used where there is overlap between two such regions.

(SEQ ID NO: 700)
GTCAGGTTCATGGTTACGAAGCTGCTGACCCCAGGATCCCAGCCCGTGGGAGAGAAGGGGGTCT
CTGACAGCCCCCACCCCTCCCCACTGCCAGATCCTTATTGGGTCTGAGTTTCAGGGGTGGGGCC
CCAGCTGGAGGTTATAAAACAGCTCAATCGGGGAGTACAACCTTCGGTTTCTCTTCGGGGAAAG
CTGCTTTCAGCGCACACGGGAAGATATCAGAAACATCCTAGGATCAGGACACCCCAGATCTTCT
CAACTGGAACCACGAAGGCTGTTTCTTCCACACAGTACTTTGATCTCCATTTAAGCAGGCACCT
CTGTCCTGCGTTCCGGAGCTGCGTTCCCGATGGTCCTCCTTTGGCTCACGCTGCTCCTGATCGC
CCTGCCCTGTCTCCTGCAAACGAAGGAAGGTGGGAAGCCTTGGGCAGGTGCGGAGAATCTGACC
TGCTGGATTCATGACGTGGATTTCTTGAGCTGCAGCTGGGCGGTAGGCCCGGGGGCCCCCGCGG
ACGTCCAGTACGACCTGTACTTGAACGTTGCCAACAGGCGTCAACAGTACGAGTGTCTTCACTA
CAAAACGGATGCTCAGGGAACACGTATCGGGTGTCGTTTCGATGACATCTCTCGACTCTCCAGC
GGTTCTCAAAGTTCCCACATCCTGGTGCGGGGCAGGAGCGCAGCCTTCGGTATCCCCTGCACAG
ATAAGTTTGTCGTCTTTTCACAGATTGAGATATTAACTCCACCCAACATGACTGCAAAGTGTAA
TAAGACACATTCCTTTATGCACTGGAAAATGAGAAGTCATTTCAATCGCAAATTTCGCTATGAG
CTTCAGATACAAAAGAGAATGCAGCCTGTAATCACAGAACAGGTCAGAGACAGAACCTCCTTCC
AGCTACTCAATCCTGGAACGTACACAGTACAAATAAGAGCCCGGGAAAGAGTGTATGAATTCTT
GAGCGCCTGGAGCACCCCCCAGCGCTTCGAGTGCGACCAGGAGGAGGGCGCAAACACACGTGCC
TGGCGGACGTCGCTGCTGATCGCGCTGGGGACGCTGCTGGCCCTGGTCTGTGTCTTCGTGATCT
GCAGAAGGTATCTGGTGATGCAGAGACTCTTTCCCCGCATCCCTCACATGAAAGACCCCATCGG
TGACAGCTTCCAAAACGACAAGCTGGTGGTCTGGGAGGCGGGCAAAGCCGGCCTGGAGGAGTGT
CTGGTGACTGAAGTACAGGTCGTGCAGAAAACTTGAGACTGGGGTTCAGGGCTTGTGGGGGTCT
GCCTCAATCTCCCTGGCCGGGCCAGGCGCCTGCACAGACTGGCTGCTGGACCTGCGCACGCAGC
CCAGGAATGGACATTCCTAACGGGTGGTGGGCATGGGAGATGCCTGTGTAATTTCGTCCGAAGC
TGCCAGGAAGAAGAACAGAACTTTGTGTGTTTATTTCATGATAAAGTGATTTTTTTTTTTTTAA
CCCAAAA
An additional CD123 isoform (NM_002183.4) cDNA is provided as:
(SEQ ID NO: 701)
CTTCGGTTTCTCTTCGGGGAAAGCTGCTTTCAGCGCACACGGGAAGATATCAGAAACATCCTAG
GATCAGGACACCCCAGATCTTCTCAACTGGAACCACGAAGGCTGTTTCTTCCACACAGTACTTT
GATCTCCATTTAAGCAGGCACCTCTGTCCTGCGTTCCGGAGCTGCGTTCCCGATGGTCCTCCTT
TGGCTCACGCTGCTCCTGATCGCCCTGCCCTGTCTCCTGCAAACGAAGGAAGATCCAAACCCAC
CAATCACGAACCTAAGGATGAAAGCAAAGGCTCAGCAGTTGACCTGGGACCTTAACAGAAATGT
GACCGATATCGAGTGTGTTAAAGACGCCGACTATTCTATGCCGGCAGTGAACAATAGCTATTGC
CAGTTTGGAGCAATTTCCTTATGTGAAGTGACCAACTACACCGTCCGAGTGGCCAACCCACCAT
TCTCCACGTGGATCCTCTTCCCTGAGAACAGTGGGAAGCCTTGGGCAGGTGCGGAGAATCTGAC
CTGCTGGATTCATGACGTGGATTTCTTGAGCTGCAGCTGGGCGGTAGGCCCGGGGGCCCCCGCG
GACGTCCAGTACGACCTGTACTTGAACGTTGCCAACAGGCGTCAACAGTACGAGTGTCTTCACT
ACAAAACGGATGCTCAGGGAACACGTATCGGGTGTCGTTTCGATGACATCTCTCGACTCTCCAG
CGGTTCTCAAAGTTCCCACATCCTGGTGCGGGGCAGGAGCGCAGCCTTCGGTATCCCCTGCACA
GATAAGTTTGTCGTCTTTTCACAGATTGAGATATTAACTCCACCCAACATGACTGCAAAGTGTA
ATAAGACACATTCCTTTATGCACTGGAAAATGAGAAGTCATTTCAATCGCAAATTTCGCTATGA
GCTTCAGATACAAAAGAGAATGCAGCCTGTAATCACAGAACAGGTCAGAGACAGAACCTCCTTC
CAGCTACTCAATCCTGGAACGTACACAGTACAAATAAGAGCCCGGGAAAGAGTGTATGAATTCT
TGAGCGCCTGGAGCACCCCCCAGCGCTTCGAGTGCGACCAGGAGGAGGGCGCAAACACACGTGC
CTGGCGGACGTCGCTGCTGATCGCGCTGGGGACGCTGCTGGCCCTGGTCTGTGTCTTCGTGATC
TGCAGAAGGTATCTGGTGATGCAGAGACTCTTTCCCCGCATCCCTCACATGAAAGACCCCATCG
GTGACAGCTTCCAAAACGACAAGCTGGTGGTCTGGGAGGCGGGCAAAGCCGGCCTGGAGGAGTG
TCTGGTGACTGAAGTACAGGTCGTGCAGAAAACTTGAGACTGGGGTTCAGGGCTTGTGGGGGTC
TGCCTCAATCTCCCTGGCCGGGCCAGGCGCCTGCACAGACTGGCTGCTGGACCTGCGCACGCAG
CCCAGGAATGGACATTCCTAACGGGTGGTGGGCATGGGAGATGCCTGTGTAATTTCGTCCGAAG
CTGCCAGGAAGAAGAACAGAACTTTGTGTGTTTATTTCATGATAAAGTGATTTTTTTTTTTTTA
ACCCA

Underlining indicates the regions complementary to gRNA D1 (or the reverse complement thereof).
gRNAs Targeting CD327 (Siglec-6)

The present disclosure provides a number of useful gRNAs that can target an endonuclease to human CD327, also known as Siglec-6. In some embodiments, a target domain sequence that can be targeted by a suitable gRNA, which may comprise an equivalent RNA targeting domain sequence (comprising RNA nucleotides instead of DNA nucleotides), includes about 16 to about 30 nucleotides of a human CD327 isoform, e.g., having the nucleic acid and amino acid sequence of CD327 (ENSG00000105492) isoform ENST00000425629.8; ENST00000346477.7; ENST00000359982.8; ENST00000343300.8; ENST00000436458.5; ENST00000391797.3; ENST00000474054.1; ENST00000496422.5; or ENST00000489837.1.

gRNAs Targeting CD312 (EMR2)

The present disclosure provides a number of useful gRNAs that can target an endonuclease to human EMR2, also known as CD312. Table 18 below illustrates target domains in human endogenous EMR2 that can be bound by gRNAs described herein. In some embodiments, a target domain sequence that can be targeted by a suitable gRNA, which may comprise an equivalent RNA targeting domain sequence (comprising RNA nucleotides instead of DNA nucleotides), includes about 16 to about 30 nucleotides of a human CD312 (EMR2) isoform, e.g., having the nucleic acid and amino acid sequence of full length EMR2 (ENSG00000127507) isoform ENST00000315576.8 (Table 19).

TABLE 18

| | Spacer Sequence | SEQ | | | |
|---|---|---|---|---|---|
| ABE | (i.e., Target | ID | | Reverse | SEQ ID |
| Guide | Domain Sequence) | NO. | PAM | Compliment | NO. |
| EMR2 ABE | CCTCTACCTGTGCAGAGCTT | 2005 | CGG | AAGCTCTGCACAGGTAGAGG | 2012 |
| sg25 | | | | | |
| EMR2 ABE | CTCTACCTGTGCAGAGCTTC | 2006 | GGG | GAAGCTCTGCACAGGTAGAG | 2013 |
| sg26 | | | | | |
| EMR2 ABE | CCTTACCTGGATGGTGTTAT | 2007 | TGG | ATAACACCATCCAGGTAAGG | 2014 |
| sg27 | | | | | |
| EMR2 ABE | TGTACCGTCACAAGTCTCCA | 2008 | TGG | TGGAGACTTGTGACGGTACA | 2015 |
| sg29 | | | | | |
| EMR2 ABE | AGCACTCACACGGTGGGAGA | 2009 | AGG | TCTCCCACCGTGTGAGTGCT | 2016 |
| sDex 13 | | | | | |
| EMR2 ABE | ATCTTACCTTGTGTTCCGGA | 2010 | GGG | TCCGGAACACAAGGTAAGAT | 2017 |
| sDex 18 | | | | | |
| EMR2 ABE | GTGGTACCTGCTGGCTGAGG | 2011 | AGG | CCTCAGCCAGCAGGTACCAC | 2018 |
| sDex 19 | | | | | |

TABLE 19
Exemplary amino acid and nucleic acid sequences of full length EMR2 (ENSG00000127507) isoform
used is transcript: ENST00000315576.8

| Identifier | |
|---|---|
| (SEQ ID NO) | Sequence |
| EMR2 amino | MGGRVFLVFLAFCVWLTLPGAETQDSRGCARWCPQDSSCVNATACRCNPGFSSFSEIITTPMETCDDINECATLSKVSCGKFS |
| acid | DCWNTEGSYDCVCSPGYEPVSGAKTFKNESENTCQDVDECQQNPRLCKSYGTCVNTLGSYTCQCLPGFKLKPEDPKLCTDVNE |
| sequence | CTSGQNPCHSSTHCLNNVGSYQCRCRPGWQPIPGSPNGPNNTVCEDVDECSSGQHQCDSSTVCFNTVGSYSCRCRPGWKPRHG |
| (SEQ ID NO: | IPNNQKDTVCEDMTFSTWTPPPGVHSQTLSRFFDKVQDLGRDYKPGLANNTIQSILQALDELLEAPGDLETLPRLQQHCVASH |
| 2019) | LLDGLEDVLRGLSKNLSNGLLNFSYPAGTELSLEVQKQVDRSVTLRQNQAVMQLDWNQAQKSGDPGPSVVGLVSIPGMGKLLA |
| | EAPLVLEPEKQMLLHETHQGLLQDGSPILLSDVISAFLSNNDTQNLSSPVTFTFSHRSVIPRQKVLCVFWEHGQNGCGHWATT |
| | GCSTIGTRDTSTICRCTHLSSFAVLMAHYDVQEEDPVLTVITYMGLSVSLLCLLLAALTFLICKAIQNTSTSLHLQLSLCLFL |
| | AHLLFLVAIDQTGHKVLCSIIAGTLHYLYLATLTWMLLEALYLFLTARNLTVVNYSSINRFMKKLMFPVGYGVPAVTVAISAA |
| | SRPHLYGTPSRCWLQPEKGFIWGFLGPVCAIFSVNLVLFLVTLWILKNRLSSLNSEVSTLRNTRMLAFKATAQLFILGCTWCL |
| | GILQVGPAARVMAYLFTIINSLQGVFIFLVYCLLSQQVREQYGKWSKGIRKLKTESEMHTLSSSAKADTSKPSTVN |
| EMR2 cDNA | AGAAAGACCAGAAAGATCCGGGGGGTCTGCTCTGTGCGGTGAAGCTTCTCTTCTTGGCACCTGCCTGGCATCGGAAGAGGGCC |
| sequence | CCTTCTCCCTCCCTGGGCTTTTATGTGGACACTGTAATGCCTCAGTTTTCTTTCTTTCTTTTTGTTTTTGACACAGGGTCTCA |
| (SEQ ID NO: | CTCTATCACCCAAGCTGGAGTGCAGTGGCACCATCTTAGCTCACCGCAGCCTCAAACTCCCCAGCTCGGGTGATTCTTCCTGC |
| 2020) | CTCAGCCTCCTGAGTAGTAGCAGCTGGGACCACAGACGTCTGCCACCAAGCCCAGCTGCACCGCCAGTTCCGGGGAGGGCCCT |
| | GGGCCAGCGGCTGTCCGCCCCCCCTCCTTTATAAAGTCCTGGCCTCGGGACAGCCCGCACAGCTGCCCAGCCTGCGGAGACGG |
| | GACAGCCCTGTCCCACTCACTCTTTCCCCTGCTGCTCCTGCCGGCAGCTCAGCTGGAACCATGGGAGGCCGCGTCTTTCTCGT |
| | CTTTCTCGCATTCTGTGTCTGGCTGACTCTGCCGGGAGCTGAAACCCAGGACTCCAGGGGCTGTGCCCGGTGGTGCCCTCAGG |
| | ACTCCTCGTGTGTCAATGCCACCGCCTGTCGCTGCAATCCAGGGTTCAGCTCTTTTTCTGAGATCATCACCACCCCCATGGAG |
| | ACTTGTGACGACATCAACGAGTGTGCAACACTGTCGAAAGTGTCATGCGGAAAATTCTCGGACTGCTGGAACACAGAGGGGAG |
| | CTACGACTGCGTGTGCAGCCCAGGATATGAGCCTGTTTCTGGGGCAAAAACATTCAAGAATGAGAGCGAGAACACGTGTCAAG |
| | ATGTGGACGAATGTCAGCAGAACCCAAGGCTCTGTAAAAGCTACGGCACCTGCGTCAACACCCTCGGCAGCTACACGTGCCAG |
| | TGCCTGCCTGGCTTCAAGCTCAAACCTGAGGACCCGAAGCTCTGCACAGATGTGAATGAATGCACCTCCGGACAAAACCCATG |
| | CCACAGCTCCACCCACTGCCTCAACAACGTGGGCAGCTATCAGTGCCGCTGCCGCCCGGGCTGGCAACCGATTCCGGGGTCCC |
| | CCAATGGCCCAAACAATACCGTCTGTGAAGATGTGGACGAGTGCAGCTCCGGGCAGCATCAGTGTGACAGCTCCACCGTCTGC |
| | TTCAACACCGTGGGTTCATACAGCTGCCGCTGCCGCCCAGGCTGGAAGCCCAGACACGGAATCCCGAATAACCAAAAGGACAC |
| | TGTCTGTGAAGATATGACTTTCTCCACCTGGACCCCGCCCCCTGGAGTCCACAGCCAGACGCTTTCCCGATTCTTCGACAAAG |
| | TCCAGGACCTGGGCAGAGACTACAAGCCAGGCTTGGCCAATAACACCATCCAGAGCATCTTACAGGCGCTGGATGAGCTGCTG |
| | GAGGCCCCTGGGGACCTGGAGACCCTGCCCCGCTTACAGCAGCACTGTGTGGCCAGTCACCTGCTGGATGGCCTAGAGGATGT |
| | CCTCAGAGGCCTGAGCAAGAACCTTTCCAATGGGCTGTTGAACTTCAGTTATCCTGCAGGCACAGAATTGTCCCTGGAGGTGC |
| | AGAAGCAAGTAGACAGGAGTGTCACCTTGAGACAGAATCAGGCAGTGATGCAGCTCGACTGGAATCAGGCACAGAAATCTGGT |
| | GACCCAGGCCCTTCTGTGGTGGGCCTTGTCTCCATTCCAGGGATGGGCAAGTTGCTGGCTGAGGCCCCTCTGGTCCTGGAACC |
| | TGAGAAGCAGATGCTTCTGCATGAGACACACCAGGGCTTGCTGCAGGACGGCTCCCCCATCCTGCTCTCAGATGTGATCTCTG |
| | CCTTTCTGAGCAACAACGACACCCAAAACCTCAGCTCCCCAGTTACCTTCACCTTCTCCCACCGTTCAGTGATCCCGAGACAG |
| | AAGGTGCTCTGTGTCTTCTGGGAGCATGGCCAGAATGGATGTGGTCACTGGGCCACCACAGGCTGCAGCACAATAGGCACCAG |
| | AGACACCAGCACCATCTGCCGTTGCACCCACCTGAGCAGCTTTGCCGTCCTCATGGCCCACTACGATGTGCAGGAGGAGGATC |
| | CCGTGCTGACTGTCATCACCTACATGGGGCTGAGCGTCTCTCTGCTGTGCCTCCTCCTGGCGGCCCTCACTTTTCTCCTGTGT |
| | AAAGCCATCCAGAACACCAGCACCTCACTGCATCTGCAGCTCTCGCTCTGCCTCTTCCTGGCCCACCTCCTCTTCCTCGTGGC |
| | AATTGATCAAACCGGACACAAGGTGCTGTGCTCCATCATCGCCGGTACCTTGCACTATCTCTACCTGGCCACCTTGACCTGGA |
| | TGCTGCTGGAGGCCCTGTACCTCTTCCTCACTGCACGGAACCTGACGGTGGTCAACTACTCAAGCATCAACAGATTCATGAAG |
| | AAGCTCATGTTCCCTGTGGGCTACGGAGTCCCAGCTGTGACAGTGGCCATTTCTGCAGCCTCCAGGCCTCACCTTTATGGAAC |
| | ACCTTCCCGCTGCTGGCTCCAACCAGAAAAGGGATTTATATGGGGCTTCCTTGGACCTGTCTGCGCCATCTTCTCTGTGAATT |
| | TAGTTCTCTTTCTGGTGACTCTCTGGATTTTGAAAAACAGACTCTCCTCCCTCAATAGTGAAGTGTCCACCCTCCGGAACACA |
| | AGGATGCTGGCATTTAAAGCGACAGCTCAGCTGTTCATCCTGGGCTGCACGTGGTGTCTGGGCATCTTGCAGGTGGGTCCGGC |
| | TGCCCGGGTCATGGCCTACCTCTTCACCATCATCAACAGCCTGCAGGGTGTCTTCATCTTCCTGGTGTACTGCCTCCTCAGCC |
| | AGCAGGTCCGGGAGCAATATGGGAAATGGTCCAAAGGGATCAGGAAATTGAAAACTGAGTCTGAGATGCACACACTCTCCAGC |
| | AGTGCTAAGGCTGACACCTCCAAACCCAGCACGGTTAACTAGAAAAATCTTCTGAATAAGATCTTCCCTCTTTGCCCGTGGAA |
| | AATCTGAACAATCTTTGAGCCATCTAGAGGGGAAAGAAAAGACTTTGTTCTGTGTGTTTCAAGAAATTCACCATGTCAGCAAT |
| | ATGAAGGATGTTATGGAAGGCGTGCTAGGCATTCAATTCCTGCAGAAACCGGAAATCTTCCATGCCCTGCAATGTGCTCATCA |
| | AACTCTCAGCATATGGACGGCCAGCTGTGGCCCATATCTTGGTCACTCTGAAGCACAATATTTATGAAGCTATAGAACGTTAA |
| | GACCTCTTTCACAGCCTCTCCTTCCTACAAAGACTCCTCCAAATCITAAAATGAAGCAGGAAAACGAGCCTAAGAGGACTTTC |
| | ATACCGACAACATCTGAAAGGACTAGAATGTTCACACCACGATCTGGATTTCTTAATTTTTTGTTTTTGTTTTTGTTGTTCTC |
| | TAGTTCTACGGGTTTGATTATTTAGTCATGTGAAAAATATTGATTACTCACACATAGATCAAGAGAGACACGGCTCCTGCCTT |
| | CATGGAGCTTTTAGGGGAAAATGAAGTGGCTCTTGCAGCTAGAGTTGACTCAGAAGCCGAAATTCCTAGAAATCAGGTTTCTA |
| | CTGCTAGGCAATTGAAGTATAAACTATTTTATAAACACTGTCTTCTTTCGTCTTCACACCAACATGCAGAAAAGTTTCTAATC |
| | TCAGATCGGGGATGTGCAACAAATTCCATTTCAAAGGAATGACCTGCAAAACTCCTAAATATTCCAAGCAAATGCCCTTAACC |
| | CTGTCTGTTATCTGCTTTCCTTGAACAGAAATTCTACATGACCATAAAACCTCGAAGATGGGTATGGCACAGTTCATGCCCTG |
| | TAATCCTAGCACTTTGGGAGGGTGAGGCAGGAGGATGGCTCAAGCCCAGGAGTTTGAGACCAGTGTGGGCAACAGAGTGAGAA |
| | CCATCTCTACCCAAAAAAAAAATTAAAAATTAGCCAAGCATGGTGATGATATAGGAGTTAAGGAGAAATCATTTAGGCAAATA |
| | GCAAGGGTAGGAAGTCCTCAGTAAGGTTTTCCATTTAATGAAAAGCAGCCCCCAAAATCATTTTCTTTTCTAACAAAGAACAG |
| | CCTGTAAAATCGAGCTGCAGACATAGACAAGCAAGCTGGAAGCTTCCACGGGTGAATGCCGGCAGCTGTGCCAATAGGAAAAA |
| | GCTACCTAGACTAGGCATGTCCAAAATGGCGGCTCCAAGTTCCCTTCTCTTTGCCAGCCATGTGTACAGTAAAAAGCAGGCAA |
| | CATAGTGTCAGCCAAAGCTCATTTGCATAATAAGATTAGGGTGGGGTGGCCAGCTCACATAGGGGTAGGCCCTAGGTAAATCA |
| | GACACCGCCTTCTCAAGCCTGTCTATAAAATCTGGTACACTATGACGAGGGTCAGATTTCCCATTCAGACGCCCCTCTCCCAT |
| | GCAAGAGAAAGAGCTGTTCTCCTTTCTCTTTCTTTTGCCTATTAAACCTCTGCTCCTGGCCAGGCACAGTGGCTCACGCCTAT |
| | AATCCCAGCACTTTGGGAGGCTGAGGTGGTCAGATCACCTAAGGTCAGGAGTTCAAGACCAGCCTGGTCAACATGGTGAAATC |
| | TTGTCTCTAGTAAAAATACAAAAATATATGAAATCTCACATAGATGATAATATTAAGTTCCAAAAGCAACTCAACCTGGTAGA |
| | TTCTAATTTTTTTTGAGGCAGGGTCTTGCTTTGTCACCCATGCTGGAGTACAATGGCACAAACACTGCTCACTGCAGCCTCGA |
| | CCTCCCAAGGCCTAAGCAATCCTCCTGCCTCAGTCCCCCTCCAGGTATTTGAAACTACAGGTGTGTACCACCACACCGGGCTA |
| | ATTTTTGTATTTTTTGTAGAGACGTGGGTCTCACTATGCTGCCCAGGCTCAGGTCTTAATCTCCTGAGCTCAGGCAATCCGCA |
| | GGCCTCAGCCTCCCTAAGTGCGGGGATTACAGGCTTGAGCCACTGCACCTAGCCTCTATTTGTTTTACAAAAGAGAAATTGAG |
| | ATCCTGAATGTTAAGTGACTTGCCTGAGGCCATCCCACTAACAGGAGCCAGGGTTAGGATTCAAACCCCATCCAACTGGTCCC |
| | AGAGCTGGAGCTTCTTGCACTGCCCTACACTACCTACCATCTCCATCCTCTGGGCACCTTTTTATAAGAACCAAAACATTACA |
| | GAGCATTGCTTTGTCAACTCAGCTGGGAACATTTCCCAGTGCAACTCACATTTTTCACTGCTCTGTGCCTGTCCGTATAAGCT |
| | CAATGAGTATTGATTTAGGGGCTTTGGAGAACTTTGAATGCTACCCCCCAAGTAACCATTGTTGGCAACCTGGTACCTCTACT |
| | TTTAGCCATTTCTCCTTCTCTATAAATAGTGCAGAAGTAACCCACTTGGTAACAGGCATCCTTGCCAAGCCTCCACCACTAGG |
| | TCAGTGTAAGAATTAAAGAAAGAGGAAAGAAACACAAAAAGTGGCTTGATGGTTAAGACAGGTTTATTTTAGAGAAAACACAC |
| | CTGAGAGGGGCTGCTGGCTGAATTAGGTTAGAGTCTTTTCTACAGACTAAGAGTGTTTAAGGATTTAGGGTGGGAGAGTTTCT |
| | TAGAGGCTTGGACTGCTTCTGTGTTTTTTTTGTTGTGCTTATATGGGAGGGAGAGTGGTGTGTTTGCTTTTATACATTTTTCT |
| | GCAGCTGTAGGCATACCCCCCAAGTCTGCTTTTAGCTTCCCTATTTTAGTGCACCTGGAGGGAAAGGAATGTGCTTATTAAGG |
| | CCCACTGTTTTACTGGGGCCCATTGTATGAGGGTGAAGTTTGGCAGTTACCCAAGAGACTTTTCCTCCACCTTCCTCTGTGCC |
| | CGAGCTGTTTTATCTGCATTTTACTGTCTGCTTTTTTTGGCTGCTTATAGTTTTTAAAAAAGTAATTTCCTTAAATCCAGAAG |
| | GCTAAAAATGAAGCTGAAACTTAAAGTGGCGGTGTTTGTCCAAAATAACGGGGCTCCTGCTCTGCCAGTCAGTACCCTCAAGT |
| | CACTCCTGATCCTCAACCTCCATGCCTAAGGCTGGTTCAAGAGACCACATAATATCTGCCTTTTATTACATACATGATGGGTG |
| | CATGGGATTCTGCGTGCCCTTTGCTTGATATAGACTGCTAAGGTGAGATGGGGAATATCAGAGTCAGCTGCTGCTTGAGGAAG |
| | CAGAACACACAGCTGGAGGCTTGGAACATGTGGGTCCCTATGAGTGTAGAGCCCATATCCCCATAGAGTCTACCTAGAGCAGG |
| | GGTCGCCAAATGTTTTCTTAAAGAGCCTGATAGTGTATATGTTAGGCTTTGTGAGCCAGGTATTTACAGCAACTCAATTCTAC |
| | CACTGTGGTATGAAAACAGCTATAGACAATCATAAATGAATGATCATGGCTATGTTTTAATAAAACTTTACAGACACTGAACT |
| | TGAACTTCCATTGTGATATGAAAACAGCTATAGACAATCATAAATGAATGATCATGGCTATGTTTTAATAAAACTTTATGGAC |
| | ACTGAGCTTGAACTTCATATATCAGTCATGTGACACAAAATATCATTCTTCTTTTTATTTGTTTTCACCCATTTGAAAAATGT |
| | AAAAACTATTCTTAGCTGTACAGAAACAGATGGTGGGTCAGATTTGACTCACTGCCCATAGTTTCCAGACCATGATGTTCAGG |
| | TTCATGGAAGCACTTCACTCTACACTAATTTATTCATCCATTCATCACATATGTGATGAGCACATACAGACACACCTCAGAGA |
| | TATTGCAGGCTCCACTCCAGATCACCACAATAATGTGAATATTGCAATAAAGCAAGTCATACACATTT |
| EMR2 | AGAAAGACCAGAAAGATCCGGGGGGTCTGCTCTGTGCGGTGAAGCTTCTCTTCTTGGCACCTGCCTGGCATCGGAAGAGGGCC |
| genomic DNA | CCTTCTCCCTCCCTGGGCTTTTATGTGGACACTGTAATGCCTCAGTTTTCTTTCTTTCTTTTTGTTTTTGACACAGGGTCTCA |
| sequence | CTCTATCACCCAAGCTGGAGTGCAGTGGCACCATCTTAGCTCACCGCAGCCTCAAACTCCCCAGCTCGGGTGATTCTTCCTGC |
| (SEQ ID NO: | CTCAGCCTCCTGAGTAGTAGCAGCTGGGACCACAGACGTCTGCCACCAAGCCCAGgtaattttttttctttttgaagaaacag |
| 2021) | agctttgccatgttgcccaggctggtctcaaactcctgagctcaagcgatcctcctgcctcggcctcccagagtgctgggatc |
| | acaggtgtggagaaataggaacgcttttacactgttggtgggaatgtaaattagttcaaccattgtggaagatagtgtggtga |
| | tttctcaaggatccagaactagaaataccatttgactcagcgatcccattgctgggtatatacccaaaggattataaatcatg |
| | ctactataaagacatatgcacatgtatgtttattgcggtactattcacaatagcaaagacctggaatcaacccaaatgtccat |
| | caatgatagactggattaagaaagtgtggcacatatacaccagggaatactatgcagctataaaaaaggatgagttcatgtcc |
| | tttgcagggacacagatgaagctgaaaaccatcattctcagcaaactaacacagaaacagaaaaccaaacaccacatgttctc |
| | actcataggtgggaactgagcaatgagaacacctggacacagggcgggaaacatcacacactggggcctgtcggggggtgggg |
| | gcctgggggagggattgcattaggagaaatacctaatgtaaatgacgagttgataggtgcagcaagccaacatggcacatgta |
| | tgcctatgtatcaaacctgctcgttgtgcacatgtaccctagaacttaaagtctaaaaaatttaattaattaattaattaatt |
| | aatttaaaaagaaagaaagaaaccgagcttcgccatgttgcccaggctggtctcaaactcctgagctcaagcgatcctcctgc |
| | ctcgccctcccagagtgctgggatcacaggcgtgagcctctgtacctggcctcagtttcctcatctggaaaacagcaggggaa |
| | gatactgccagctgggacgcaggctctgccaactctggcatgccaaagctctgggcacagggtgagccctaccaaagtgggag |
| | cccagagctatttctgacaaggcccctgcgtgcctgcccagggaagagccaagtggccccaggcgagaccgccccgcccggct |
| | gccacaccttcccctgcacagcagccagcctggaacacacagaggccagagggttggtcagagccacatggtggaaactccag |
| | cagagggatttttaaagcaggttgcacttcattcttgctgagcgagtacacgtgtgtgtgtgtgtgtgtgtgtgtgtgtgtgt |
| | gtgtgtgcagcgcccctggttctgtgtttttattgtgctttcctgctggcttccagCTGCACCGCCAGTTCCGGGGAGGGCCC |
| | TGGGCCAGCGGCTGTCCGCCCCCCCTCCTTTATAAAGTCCTGGCCTCGGGACAGCCCGCACAGCTGCCCAGCCTGCGGAGACG |
| | GGACAGCCCTGTCCCACTCACTCTTTCCCCTGCTGCTCCTGCCGGCAGCTCAGCTGGAACCATGGGAGGCCGCGTCTTTCTCG |
| | TCTTTCTCGgtaagtactttggggccccgctgggggtggtagcgaggaagctccagcgggaccccttggatgcgtctgagaaa |
| | cgggaggcgccgctggtgtctgtggggccggcgatggagaaagccacatggtcaagcaggcactcacgggcacacggcgagag |
| | gggctggtggtgcttgggcacccccacaccccggcaggaactccagggtgctctcggagcctgctaccctgcccggagggatc |
| | ctcaagctttctactggagccagtttggcatctgctccataagaggggaaactgaggcacgggggaaggaaaggggcaaaaag |
| | tgagtgctaggaccccagagccactgctcttaatatagcgcatagaaagacatgggcggggggcggggaaaatagaggccagg |
| | acttctccactgcagggtacccccagcttctcccttctttctctctctctctctgtctttctccttctgtctctctttcagtc |
| | tctctatctccctaatctccgttttctctctacctctgcctctttgtctctctctctggtgaacaacaaggagatccctaggt |
| | ccccatctctaaagcccctggtgacctgaggagggtcccccaggacatgccttggaaaggggaacttcctgagtgctgggaac |
| | cgcagcgccacatgcaccccaacaggcggccacagcgttttcctgtgggaggggcgcccggggtgaaatatcaccactgggtt |
| | tcaggatgggccgccagggctggcggcggttgtggtgtgtgtgcacctgtgtccctgaggaagcatgcggttccagggcatct |
| | tagagcctgggggatccagcgccatcatttttttttttttttttctgaggcggagccttgctctgtcgcccaggctggagtgc |
| | agtggcacgatctcagcttgctgcaacctccgcctcctgggttcaagcgattctcctgcctcaatctcccaagtagttgggta |
| | acaggagtctgccaccacgcccagctaatgttttgtatttttagtagagacggggtttcaccatattgggcaggctggtctcg |
| | aactcccgacctcagatgatctgcccaccttggcctcccaaagtgctgggattacagtcccgagccaccgtgcctgaccatgc |
| | cttcatttttatctaaagaaatgaagatggcagtaaatgctcaggcacaccagacagcccccaagtcagaagagcggcagctg |
| | gagctgagacccccaccaggctcatggccctttcctactcctcagttccttaaaccccacccccaagccaagctagggaggct |
| | gaggcaggaggatcgcttgaggccaggagttcaagatcagcctgggcaacagagcaagactctgtctctaaaataacttttta |
| | aattatttttaatttttttggccaggcgtgatggctcatgcctgtaatcccagcactttgagaggccaaggcgggcagatcac |
| | ctgaggttaggagttcaagaccagcctggccaacatggtgaaaccccatctctacttaagaaatacaaaaattagctgggcat |
| | ggtgggtcatgcctgtagtcccagccactcaagaggctgaggcaggagaaccgcttgaacccaggaggtggagattgcagtga |
| | gccaagattgcaccactgcattccagcctgggcaacaaagggagactccatctcaaaaaaaaataaataataaaaataaataa |
| | atcattttaaaatttttgagctgggagccgtggctcacacctgtaatcctagcactttgggaggccaaggccggcagatcacc |
| | tgaggtcaggagttcgagaccagcctggccaatatggtgaaaccccatctcactaaaaatacaaaaaattagccaggcaccat |
| | ggtgggtgcctgtagtcccagctatttgggaggttgaggcaggagaattgcttgaacccaggaggtagagattgcaattagcc |
| | gagatcgcaccattgcacttcagcctgggcgacagagcaagaccctgtctcaaaaagaaagaaagaaaatttaaaatatggcg |
| | gtactcacctgtggtcccagctactcgggagggtgaggcagggggatcgcttgagcctaggaattggaggctgcagtgagcta |
| | taatcgcatcactgcaccccagcctgggtgacccagagagaccctgtgtcaaacaaaacaaaacaaaacccacccccatgagg |
| | acagggtgacagactctctggctttgagcatccgagtgggacgtgacatctgctcactctgtttgaacgatgggcaggatctg |
| | agcctgagaggggtcagcatcctcctcccacaaagtgcatcacccttacgcctcctttcccaccctgggatcccctctgaccc |
| | cctttcctttctctgttgcagCATTCTGTGTCTGGCTGACTCTGCCGGGAGCTGAAACCCAGGACTCCAGGGgtgagtctgct |
| | gggaagcagaaagcacagtccacagccagagcctggggagggtcctggacccccgcccagcccccttcagcccagggaaagag |
| | agggctcgtgcacgggaaactcagcgctctgccccatctcccccagtgccccctttttgtgtattcccttacccctcaccttc |
| | tgaccgtgctccctgctcttgcagGCTGTGCCCGGTGGTGCCCTCAGGACTCCTCGTGTGTCAATGCCACCGCCTGTCGCTGC |
| | AATCCAGGGTTCAGCTCTTTTTCTGAGATCATCACCACCCCCATGGAGACTTGTGACGgtacagaggcttgagggcagcgcag |
| | gggacatctgcgattatgaggcatagcccagtgccagtgggggacagaggttgttgtgaggggccacagccttaccttccaga |
| | ctatcatcatggccagagaaaagagaaagagggcaggtgtggtggctcccagcactttgggaggcagaggcgggtggatcatt |
| | tgaggccaggaattcaagaccagcctgggcaacatggtgagaccctgtctctacaaaaaatacacaaaatagccgcgcatggt |
| | ggcacacatctggagtcccagctactcagaagactgaggcgggaggattgtttgagcccaggagtttgaggttgcagtgagct |
| | atgattgcaccattgcactctagcctgggcaacagagtgagatcctgtctcaggaaaaagaaagaaacaaagaaagaggccag |
| | gtgtggtggctcatgcctgtaatcccagcactttgggagggattgtctgggcaacaggagcaagaccccatctcaaaaaaaaa |
| | aaaaaaagacagaaagagagaaggagagaaagaaaagagaagaaaagaaaaagagagagagggagggagggaaagaaaaagga |
| | aaggagggagggagggagggaggaaagaaggaaggaaggaagggaggaaaaaggaaaaaatatgaagggggagaagtaagaag |
| | tgaataggcatggcttcctggagagagagaagctgcgtgctcaggaatctggagtctgtgcctcagtttaccgttaagactgg |
| | gaagggggtacattctggccataggtttttttttctcccttatttttcttttttctttttttttttgtttttttttttttttt |
| | ttgagatggagtctcactctgtcgctcaggctgaggagtgcagtggcgcgatctcggctcaccgcaacctccacctcccaggt |
| | tcaagcgattcttctgcctcagcctcccgagtagctgggattacaggcatgcaccaccatgcccggctagtttttgtattttt |
| | agtagagatggggttttgccctgttggcaggctggtctcgaactcctgacctcaagtgatctgccggcctcggcctcccaaag |
| | tgctgggattacaggtatgagccactgcacccggctgccatggggtttaaatttctacaaaaagactggggatggggatatgg |
| | cgcttcctgtccatcccccacctcagcactgtccactgtgtccaaatctaggatgccagccagagaactgagatcaagtgtgg |
| | tcttcaggaatggcctgctccagctgcagcatagcctgtgcagtgcaacttgagatcctttccaaaagatactaagcagcagg |
| | ccccatgttggggagtcaggacgggggtctctgccctgacggaactcacatctttgggagatgacctcctgtcctgttgtgtt |
| | ccagACATCAACGAGTGTGCAACACTGTCGAAAGTGTCATGCGGAAAATTCTCGGACTGCTGGAACACAGAGGGGAGCTACGA |
| | CTGCGTGTGCAGCCCAGGATATGAGCCTGTTTCTGGGGCAAAAACATTCAAGAATGAGAGCGAGAACACGTGTCAAGgtaaga |
| | accaccccacatcctccatcaccactgtccatgaggtttggggtcaccagagctgtttctgcagcatccagggagcaggtacc |
| | caagtataggttcagttcctggagtctgagatgagacaggtgtgcatgtacctgtttcgccagcccagagaggcagcgtggtg |
| | tgaaggccaagggaaaactctgggaccagctacctgggcccaaatcctggctttgccacctgttacctaaatgatattaccaa |
| | catcactactgctcttagagccttggtttacccatctataaaatgaggatgataataatggcctactcctatgccttctagga |
| | ggaccaaggaaagaataggtgcttctaacagagcccagcacacagcaggtgctacaggcatcttcattctttaaacgttatta |
| | tgcactgtaatcccagcactttgggaggccgaggcggatggatcacggggtcaggagatcgagactatcctggccaacatggt |
| | gaaaccctgtctctactaaaagtacgaaaattagccgggtgcagtggtgggtgcctgtaatcccagctactcaggaggctgag |
| | gcaggagaatcacttgaaccagggagtcggaggtttcagtgagcggagatcgtgccactgcactccagcctggtgacagtgca |
| | agactccatctaaaataaataaataaataaataaataaataaataaataagcaatggatgccttttaggagttgttcagacaa |
| | gctgggctcctgtggggccacactcctggctggcactcagaagaaatctatttcgacacagcctgcctcatcccttgccatgg |
| | tcttattccacactgcagctccagcccatgagaagccaggcccccatgcatcactctgctttgccttctttccagtgggaact |
| | agttgggtgcgggagggagcttcagacatctgcaaggccagatgggaaccagtagcaggtggacagagaagggagattaacac |
| | agagtacacagagtgggaagggaaggtgagcaaaggcaggaaatgaagaagctgaggctgagccaagggtcccaggggacatt |
| | aagggggttcagcagacctgccctgctgtaaagagaaaagtggcagtgctcctgttcccagccctgtccctcctgttctccct |
| | gcagtcagtagagcctccctggtgggaggaaagtgaggacagagcagaggtgtacgttctgagagcacgcagctgcatagagc |
| | ccagcaggggggctggttcacaggggtcccctgtgaacagatgctacattgcatgcacatatcttgaccaaaggacagagcct |
| | tcctgtgaattagaaaaaggactctccgggccaggcacagtggctcacgcctgtaatcccagcactttgggaggctgaggcgg |
| | gtggatcacctgaggtcaggagttcaagaccagcctgacaaacatagtgaaaccccatctctactaaaaaatacaaaagttag |
| | ccaggcatagtggtgagcacctgtaatcccagctactcaggaggctgaggcaggagaatcacctgaacccagaggcagaggtt |
| | gcagtgagccaggatctcaatattgcactccagcctgggtgacagagcaagactttgtctcaaaaaaaaaaaaaaaaaaaaaa |
| | aaaaaagaaaagaaagaaacaaaaagaaaaagagaaacagaaaagcctctcctctccaggtagacacagccccatttggggaa |
| | tggagaggttttcagcctctgctcaccgccttggctgggcatccatatgtagtcagcaacctacacaactgtacgtggtggtc |
| | atgggtccttgtcagtgagagcctctgttttagtctatgttgtgttgctataaaggaatacatgaggctggatcatgtataaa |
| | gaaaagaagctgatttggctcatggttctgcaggccatgtaagacacatggctccagcatctgcttctggtgagggccttagg |
| | gagcttccactcttggcagacgggaaaggggaagctggcatcccatggcaagaggaaggaagcaagagagaagggaggagata |
| | ccaggctttttccaacaagcagttttcatgggaactaacaaggcgagaactcactcatgacccaaggacagcaccgagatgtt |
| | catgagggatctgcactcaggacccaaacacctcccacgaggctccatctccaacactggggatcacatttcaacgtgagatt |
| | tggaggggacaaatatccaaactgtatcaccgcatgcacaggtggtgtttgtaattattgaggaggtgtgttgccatgttaga |
| | aacaggccagcactggccgggcgcagtggctcatgcctgtaatcctagcactttgggaggccgaggtgggcggatcacaaggt |
| | caggagatcgagaccatcctggctaacatggtgaaaccccgtctctactacaaatacaaaaaaattagctgggcgtggtggtg |
| | ggtgcctgtagtcccagctaatcagagactgaggcaggagaatggcatgaacctgggaggcggagcttgcagtaagccgagat |
| | ggcaccactgcactccagcctggacgacagagcgagactccgtctccaaaaaaaagaaaagaaaaggagagaagagaagagag |
| | aaaagaaaagaggccaacactgagagagggggcttagttggtggataggcagagacacagatgagcaggaatgaagtgggaga |
| | gggctctcatggaccccatccgcacagaacagcctgctagttcggagcaggacctgcctcttgaagcctcagctcagggaagg |
| | gctccgtggggccctgtctgcatccccctcacccctcagcattcttgttttttttgtctacctggtgtcacaggcctcatagg |
| | caggagacctggggccactccctcagctccttgccccacttactccaggcatcacagggccgtcccaaacacctggtcacctg |
| | tcgctatccaaacctcaaggttcagaaccctccacagagggtcacttccaaaagctcagaacatccccctgcccatctggcat |
| | cttatttgtttgttttgttttgttttattagtaaagatggggggggggtgtctcaccatgttgtccaggctggtctcgaactc |
| | ctggcctcaagtgatccgcccgtctcagcctcccaaagtgctgggattgcaggcgtgagccaccatgtccgacctggtatctt |
| | atttgtacaaaacaaaaagggggctggcactcaggatattcgatgagctcactgcgtggtgcattccagcgcagtatccattc |
| | tctctgctatcatcagcgactgccaggatgagacagtctccacacagctctgctcacctggaactctctctgatcccccagct |
| | catcatggacccaggcaccctgtccagaaatgaaggaggagggagatttgaaggcagggccactgtccccagtcgaacctgcc |
| | actggccaacaaccccacctggagaagatgcctcgtggagccctgacctctttttctgccactcagtctctgtccctttcact |
| | gagcgagactgccctgacccttcctgatagttttggtccacctgagtgcactggaggatgttgtggctaacggtgggcacagt |
| | gacatggactccccatacttggagagttgagtgaatggaagaggcactagtgtggacccaatggcaagagaggttaacatcag |
| | ggccattgctccaagcctgatcctctcccaggaggtcaacccagatcccatgggagtggaaccattcagaaataggtctgtgt |
| | ccctggtggaatgccaagagatgagatgacagatacacctccatctttgcccatcagcatctgtgcccctctccagccactaa |
| | accttcacccagggccagtctttctcagctatggattctcactacatcctgccatttccaagacaccttcctgaggatcaaaa |
| | aaaggcaccttcacagccctggggcaacccagctgtatgtcggtccccactgtccaccctggcctgggacccactgtccaggc |
| | agccctggcctatgcagcaccagactcatgctcccctggggactggctcaggggccagtcttggtccatgtgcctggagctgg |
| | gctctcagagctagtgtggatgggagagcaccatcctgctagggccaccgggattctgataactcagccagtgagcagctgtc |
| | ctggggaaatcaaggccaccagaagcagatgctggagccatgcatcttacacagcctgcagaaccatgagccaaataaacttc |
| | ttttcttgcaacacaaaacggactaaaccagagggttctcactgagaaggacccatgaccaccatgtacagttgcataggctg |
| | ctgactgcatatggatgcccagccaagagggtgagcagaagctgaaaacctgcctactctccattcctcaaatatgactgtga |
| | ctacttgaagaggagatgcctttttccaattcacaggaaggctctgtcctttggcccaggactgccactgggaaccaagacag |
| | gtcctacctgggcaagcagcatgccccctgcacttggtcccaagaaaatcccaccctgacctttgtcctgaatcctcaggctg |
| | ggacatccctgccccagctgggagctgcccagggaggcaggggagatggggaggactcagatgcttccagccatatctgaaca |
| | acagaaggcctgggccaccatgctcaccatgggacacctctgtgtggattagaaaagggaggagatggccaggcatggtggct |
| | catgcctgcaatcccagcactttgggaggccgaggtgggctgatcacgaggtcaggagatcaagaccatcctggctaacacgg |
| | tgaaaccccgtctaatactaaaaatacaaaaaattagccgggcatggtggcgggcgcctgtagtcccagctattcgggaggct |
| | gaggcaggagaatggcgtgaacccgggaggcggaggttgcagtgagttgagatcaggcaattgcactccagcctgggtgacag |
| | agcaagactccgtctcaaaaaaaaaaaaaagaaagaaagaaaagaaaagggagcagacagtggtgccttccagatgcttccgt |
| | gccaggaaacatggcggaccctcagccccgaaagctgctcacagctacgggcattcttcaccctctccttcctcttgcagATG |
| | TGGACGAATGTCAGCAGAACCCAAGGCTCTGTAAAAGCTACGGCACCTGCGTCAACACCCTCGGCAGCTACACGTGCCAGTGC |
| | CTGCCTGGCTTCAAGCTCAAACCTGAGGACCCGAAGCTCTGCACAGgtagaggccccaggaagacgctgtgaggctggacggg |
| | agctggggatggagctgagtcaggtcctccaaagcagccgaggaggagggagaagatccgcaggttcccacaaggtcaaggac |
| | ctgctaagcccctgcctaaggattcacctcccaggaaggactcgccacacggcagggaggcggcagggccttcagggcttgga |
| | gtgcccttgtgggcccccagacctcaccccttcctcatctgtcacgtgcgagtggaagaaggtgtcacttccagttctaagaa |
| | ggggaacctccaccatagtgagggagggagcaggagagagattttagtcagaaagtccagctcccatgactcagtttcccttt |
| | tggtttcacctcctataaactggggaccatggtccctgctgtgcccatctggtaggaagaccatcataagatgctttgaaggt |
| | gaagttgaaggtcaccaagtggtggcacgtgcaataatggtcttcagtctgaggcaatgaaaagataggggagtgggtgcagt |
| | gagtggagggcaggcccaggctggtcagggacagggtttgaccctctggctttgtcctcagATGTGAATGAATGCACCTCCGG |
| | ACAAAACCCATGCCACAGCTCCACCCACTGCCTCAACAACGTGGGCAGCTATCAGTGCCGCTGCCGCCCGGGCTGGCAACCGA |
| | TTCCGGGGTCCCCCAATGGCCCAAACAATACCGTCTGTGAAGgtcgagagctcagatcccacgttcccagagacccacaaaca |
| | tctgatcacatgttcaacggcgcccacacaaaccaagcagaatgagcgctggaggcgcccgactgtgtcaggcgttcattctt |
| | ctgaggctagatgagaaaagagcaagggtcctgcggaaggagctggggtactgaggggggaggctcagggggaccccaggcaa |
| | cagctgatgactcactgggaggaaggcgtttcaccatattcataacctgctcatctgcacggggcccacctgctgtgcccagg |
| | cctctccacgcttccataacccagcgtccacctctccaaggggggcactaatgccgggaggaacgagctgggggcacagacag |
| | gagacaggaccctctccaggctgggacaggacctgacccccttcttcctgtcctcagATGTGGACGAGIGCAGCTCCGGGCAG |
| | CATCAGTGTGACAGCTCCACCGTCTGCTTCAACACCGTGGGTTCATACAGCTGCCGCTGCCGCCCAGGCTGGAAGCCCAGACA |
| | CGGAATCCCGAATAACCAAAAGGACACTGTCTGTGAAGgtatgacctggccctagaagctccccacccccagcacacacactg |
| | acacgctcccgcctaatgagccgcttgtcttgttccctacagATATGACTTTCTCCACCTGGACCCCGCCCCCTGGAGTCCAC |
| | AGCCAGgtgagtggcccccacagggacgaggcggcgggaactccatccacacagcactgcatccgtctccttgttctaaactt |
| | cccacccgccgtccaggctctctgacccccacatctcctctctctgcagACGCTTTCCCGATTCTTCGACAAAGTCCAGGACC |
| | TGGGCAGAGACTACAAGCCAGGCTTGGCCAATAACACCATCCAGgtaaggacaggacccagggcaagggggcgaggcaggaag |
| | gtggctgcatcccacagaggcctggggcagtttgggtctgggaggggacaggacccaatgcagtgggtgctggtctgactccc |
| | agcatctttcagggctggtggaagctaatggacacccaagtgcacttaatatctttcctcttgctttccctggactttgggtt |
| | taggtcagcattgatattgcacagaagtgttggcctctgaggggccataaccagagtcaaggatcacctagggaatcccatcc |
| | aatgtcattttttttttagacggagtctcactctgtcgccagtccagagtttagtggcccgatctcagctcactgcagtctct |
| | gcctcccgggttcaagcgattctcctccctcagccccctgagtagctaggattacaggcacacgccaccacgcccggctaatt |
| | tttgtatttttagtagagacggggtttcaccatgttggccaggatggtctcgatctcctgaccttgtgacccaccctcatcgg |
| | cctcccaaagtgctggaattagaggcgtgagccaccgcgcccagccgccaatgccatcttcatcccccagatagacagtctct |
| | aggatctgttccctggggctgagcggttggagtcttcatgcgggccctctggcccatggctcactaggtctgtgtccacatcc |
| | ctccagAGCATCTTACAGGCGCTGGATGAGCTGCTGGAGGCCCCTGGGGACCTGGAGACCCTGCCCCGCTTACAGCAGCACTG |
| | TGTGGCCAGTCACCTGCTGGATGGCCTAGAGGATGTCCTCAGAGGCCTGAGCAAGAACCTTTCCAATGGGCTGTTGAACTTCA |
| | GTTATCCTGCAGGCACAGgtaggtccctgggtctgccccagactccagctctgcatgttttctgtctccttcctttcccagtc |
| | ccaccagagccaagtgaccacacctgtatatcagtgttactctcatcgacaaactaaaataagagattaaaaagaaatacata |
| | ccagtctggacaagatggcaatactcaatctctacaaaaaaaaaaaaatcttttaaattagacagatgtgatggcacaactgt |
| | ggccccagctagttgggaggctgaggtgagaggatcgcttgagcccaggaggttgagtctgcagtgaactatgattgcatcac |
| | tgtactccagcctggttcacagggcaagaccccatctcaaaaaagagaagacgaagaggaggaggaggaagaagaagaagaag |
| | agaaggaggaggaggtgaagaggaggaggaggataaagaggaagaaaaaggaggaagagggcaggagggggaggacagaagga |
| | ggaggacaggaggagaggaggggaggaggaagaggaggaaagaggatgggaggaggaggacaagaggaagaggacgggaggag |
| | gaggacaggatggggaggagaggaggaggagagaaggaggaggagaggaggaagaggaggaatggagaaagagaggaggagga |
| | gaaaaggaggaggagaggaggaggaggagaggagaaggaggaggaatggaggaggagaggaggcagaggaagataggaggagg |
| | agaaggagaagaggaggagaagagaaggaggagaggaggaggagacgagaaagagcggaggagaggaggagaggaggaggagc |
| | acaggaggaggaggagaggagtagaggaagaaggaagaatagaaagaaggaaaggaaaggaaaaagaaatgcataaataacat |
| | gcattttcattgtaatatgaattatcagcagtaaataattatgatgaagaatttaaaataacaaaatagaaatcacttcaaaa |
| | gagaacaatttatttaatttggaatactataaattaatatcatttgatctctaagtatgatttctttcttgtttgtttttgtt |
| | tgttttgttttgttttgttttgtttttttgagacagagtctcactctgtcgcccaggctggagagcagtgacgtgatctcggc |
| | tcactgcaagctccgcctcctgggttgacagcattctcctgtctcagcctcccgagtagctgggactacaggtgcccgccacc |
| | acgcccagctaattttttgtatttttagtagagacggggttgcaccgtgttagccaggatggtctctcgatctcctgaccttg |
| | tgcataatccacttgcctcggcttcccaaagtgctgggattacaggcgtgagccaccacgcccggcctgcataataattttta |
| | aagcatgaaaatgcatatagcattaattttattaagataaaattcacataacatcaagttcaccactttaaagtgtctgtttc |
| | acgtggggcgtgcctgtaatgccaggcgctcacacctgtaatcctagcactttgggaggctgaaatgggtggatcacttgagg |
| | tcaggagttcaaatccagcctggccaacatagtgaaaccccatctctactgaaaatacaaaaattagacaggcgtggtggtgc |
| | acacctgtaatcctagctactcaagaggctgaggcaggagaatcacttgagcttgggaggcagaggttgcagtgagccaagtt |
| | tgcaccactgcactccagactacgtgagacagagtgccactccatctcaaacaaaaaaattaaaattaaaaataaaaataaga |
| | aaaaattttaaaagcgtgtgtttcggtagaatctagtatattcagaaagttatacaaccgtcacctctatttctaaaatgctt |
| | gcatcagcccaaaagtgaacactgcacatggtaagcaaccactcctcattcctcccttcctccaacccatggcagccgctaat |
| | ctgctttctgtttccatggattttcctaatcaggatatctcacataaatggaatcatacaatatgtgaccatctagtctagct |
| | cctttcacctagcataatggttttgaggttcatgcatgccatagcatttatcaatatttcattctttttcatggctgaatact |
| | attctattgtatgaatacatcactttttttttttttatccatccatccattgatggacatttgggttgtttccaccttttggc |
| | tgttgacaaaaagagtcaaactctgtaaaatatttgaagagatttattgtgagctaaatatgaatgtatgactgaccatggcc |
| | cgtgacacagccctcaggagcccctgagaacatattcccaagtgaagtggtgttgttttctggggtaatacctgaggttcatt |
| | gcctcatgccaagaaaattaaggacacagacacacaccaggagtgagtttatgagtggaggtttaataggcaaaagaaagaga |
| | aaggagaacaactctctctcttgtgagagaaaggaatgcccaaatgggacttcccacccatgccagagggcaccggattttat |
| | agacaggcttgaggaggcagtgtctaatttacatagggcccaaagattggttggaacaggtgtgacgtttacataaggtgcag |
| | ggaagctggctgccccaccctaatcttattatgcaaatggagtcttcgccagctccttactgtatacgtggttggcaaagaga |
| | tgggaagatggagccgccattttcaacatgcctagtcccaggtggccttttcctattggcacagctgccagcattcacctgtg |
| | caggcttccagcttgcttgcctatgtctgcagctcgattttacaggctgctctttgttagaaaaaaaaaaaatgatatagggg |
| | ctgattttcattaaaaggaaagacttacccaggacttccttacccacactctctgcctaaataattcctttttaacttctata |
| | tcacaaggtagtcgtggtacagcttgttgtgattgttgttgttgttgtcttctgttggagacagagtcttgctctgtcaacca |
| | ggctggagtgcagtggtgcgatcctggctcactgtagactccacctcccgggttcaagcgattctcctgcctcagcctcccaa |
| | gtagctgggactacaggcgcccgccaccacgcccggctaattattttgtgtgttttcagtaaagacagggtttcgccatgtta |
| | accaggatggtcttgatgtcctgacctcgtgatccgcctgcctcggcctcccaaagtgctgggattacaggcgtgagacacca |
| | cgcctgcccccgcaacttggttttatacattttagggggacatgagacatcaatcaaatatgtttaagctatacattggttca |
| | gtccagaaatgcaggacaatttgaagtggaggtgtggggcgggtgcttccagattataggtagatttaagcttttctgattgg |
| | cagttggttgaaggagttattatccatagaaaagaatgtctggaatacgataaggggctgtaaaggctgaagttgtatcatcc |
| | agatgaactcttcaggtatcaggcttcagagagaatagatggtaaatgttccttattaagcttcaggtctgtgttatgttaaa |
| | tgctggtcagcttttcctcaactccaaaagggaggagggcatgacacatgtcagaccccccacttcccaccatagcctgaact |
| | agtctttccagttaaatttagagtgccctggccaaaaaggaagtccattcagatggttttgaggggggcgggagaggacttcg |
| | aattttttttttttgtttagatggctggtgtgaatagtgctcttatgaatattagtggacacattttgggttgaataactgtc |
| | ttcagttctttggggtatatactaaggagtggaattgctgggtgatatggtaattctatgttcagcttttggaggaaccacca |
| | aactttttccaaagcagctgcatcattctaattccccaccagaaacatacacgggttatcctctccacaccttcagcaaaact |
| | tgttacttccgttttcttttgttctgtttttaattgcttttattgatacataaatcttacatatttatgggtacatgtgatac |
| | tttgttgcttgcctagaatgtgtaacgatcaagtcagggtatctcaggtatccaccactttgagtacttatcatttgtatgtg |
| | ttgggaacaattgaagtcctcttttgtaactactttgaaatacatcatacagtcttgttaattatagtcactctgctctgctg |
| | tcaaacaatagaacttgggccgggcacggtggctcacgcctgtaatcccagcactttgggaggccgaggtgggaggatcacga |
| | ggtcaggagatcgacaccatcctggctaacagggtgaaaccccatctctactaaaaatacaaaaaattagccaggcgtggtgg |
| | cgggtgcctgtagtcccagctactcgggagactgaggcaggagaatggcgtgaacctgggaggcgaagcttgcagtgagccaa |
| | gatggtgccactgcactccagcctgggtgacagagtgagactccgtctcaaaaaaaaaaaaaaaaaaaaatctgcttaataag |
| | cggagaggtggctggcttggtggctcacacccgtaatcctagcactttgggaggtcaaggcaggcagatcacctgacatcagg |
| | agtttgagcccagcctggccaatatggtgaaaccctggctattaaaaatacaaaaattagctgggcttagtgacacgtgcctg |
| | taatcccagctacctgggaagctgaggcaggagattcgctggaacccaggaggcagaggctgcagtgagctgagatcgtgcca |
| | ctgcactccagcctgggtgacagagtgagactccgtctaaaaaaaaaaaatatatatatatatatgtataacttatacttttt |
| | gtctaactgtatgtctctacctgttaactcacctctcttcatcccagtgatacaggagtgaaaaagaaattatttaggcagtt |
| | agcaagggtcagagagtcctcagtaaggtttcccttttaataaaaaccagcccccaaatcatttcttttctaataacgagcag |
| | cctgaaaattcgagctgcagacatagaaaagcaagctggaagcttgcacgggtgaatgccggcagctgtgccaataggaaaag |
| | gttacctgggggcccgacatgttcaacgtgggggctccctcttcctttatctttgtcaaccacgtgtacagtacagaagcagg |
| | caacgtggagccagccaggtagagaatccatttgcataataaaagattaggctggggcagccagtttcttcatgctgtatgta |
| | aatggctcacctgttcagaccaatctttaagccttatgtaaatcggacaccgcctacccaagctcatctataaaacttcgtgc |
| | atttcaccacgcaaccggaagacccacttgggagcccctgtctctctgcaggagagggagttattctctttttcctttttttt |
| | tttttgagacggagtcgtgctgtgtcccgcagactgcagtgcaggggcgcgatctcggctcactgcaagctccgcctcccggg |
| | gtcacgccattctcctgcctcagcctccggagtagctgggactacaggcgcccaccaccatgcccagctaatttttttgtatt |
| | tttagtagagacggggtttcaccgtgttatccaggttggtctcgatctcctgacctcgtgatcctcccgcctcagcctcccaa |
| | agtgctgggattacaggcgtgagccactgcgcccagccagagctattctttttttctttcgcctattaaatctccactcttaa |
| | actaacttcttatgtgtccacatcctcaatttccctggtgtgaaacagtgaaacttgggtatttacaccacttcactagtgag |
| | atgatacctcattgtggttttgatttgcccttccctgataactaatgatgtttaacatcttttcatgtgtttgctcaccattt |
| | gcatgtcatttttggagaaatgtctattcaaatcctttgcctatttttaattgtgctgtttgtcttttggttgtccagttgta |
| | agcattctttaaacattctgatagtagatcatctgagatgaatgatttgcaaaatattttctcccgttctgtagattgtcttt |
| | tcattttcttgtcttttcactttaatgaatagaagttttaaatcttgaagacatttattttatctaatttttcttttgttgca |
| | tgtgcaattggtgtcatatttaagaattcattgacagggggtggtggctcacgcctgtaatcccagcactttggaaggccaat |
| | gtgggcggatcacctgaggtcaggagtttgagaccagcctggccaacaaggcgaaaccctgtccttactaaaaataaaaataa |
| | aaataattaaccagatgtgttggcacatgactgtaatcctagctactcaggaggctgaggcaggagaattgcttgaacctggg |
| | acacggaggttgcagtgagctgagattgtgccactacactccagcctggatgacagagcaagactctgtcccaaaaataaaaa |
| | taaaaaaagaattcattaccgcatccaagaccaggaagatttacccctgtttcctcctaagtgttctagttttagcttctaag |
| | cttaggtcactggcttattttgagttaatttttgtttgttgtgtgagggtagggttaaaacttccttattttcatgttactat |
| | ccagtaatcccagcactatttgttgaagagattattctttcccccattgtatagtcttggcaccccttgttgagaatcaactg |
| | accataagatggatgggtttattcctggactctcaattctattctgttgtgtccttatgccagtattactgtcagtttgtagt |
| | aagttttggaatcaggtagtgtgaattctccaaccttcttgttcttcttcaagattgtttttgggtatttggattccttacaa |
| | caccatatgaattttaagatgaacttttcttattctgcaaagtaataataggctaaaatattgatagggattgcattgagtct |
| | ctagaccactttgggggagtattgttatcttaacaatagaaagtcttccaatccatgaacataggctgtctttccatttagag |
| | agggccttttatttctttcaaacacttttgtaggccagaagcagtggctcacgcccaatattgtcagaggccaccaaggtggg |
| | agaattgcttgagcccaggagttcaagcccagccttggaaatatactgaaacatgatctccacaaaaaatttaaaaattagct |
| | ggctgtggtagtgagcacctgtagtgccagctacttgggaggctgaggtggaaggattggttgagcccaggagactgcagtga |
| | gctatgatcatgccactgcactctagcctgaccaacaggtgagaatttgtctcaaaaaaaaaaaaatactcaaaaaactttcc |
| | tataagtcttgagtctccttcgtaaaatgtattcctaacatttttgctgaaaatttttgcatctatgtccataagaaatattg |
| | gtctataattttattttcttgtgatgtcttcatctggttttgttatcagagcaatattagactcataaaattacttaaatagt |
| | gtttcctcctcttccatatttttaaaaaggtaaaagattcatgttcattctttcttaagctattggtagaactcaccagtgaa |
| | gtcatctggtctgggctgttagttttgagaggttttgattactgatttaatctgttataggtctgttcagatcttctatttct |
| | tcttgagttgatttcagtggtttgtgtgtcactaggaatgtgtccatttcatctaggttacctactttgtagatatacagttg |
| | ctcacactattctcttccgatattttgtaacttcagactatatatgtatatagtatatatattaagtcatagtatatcaacca |
| | ctgcaggcacgattcctaacctaaccccccttatgtaattcccacaactcatagAATTGTCCCTGGAGGTGCAGAAGCAAGTA |
| | GACAGGAGTGTCACCTTGAGACAGAATCAGGCAGTGATGCAGCTCGACTGGAATCAGGCACAGAAATCTGGTGACCCAGgtaa |
| | tggctgagatggggaggtggtcagtgaagcttcatggtgggaaaagatgcccaagaatgtgaaaaataagaaatttaaagatt |
| | aaagggaagtgtgggatgtttggagtggggctgtaagaggagagtccagctgaggaggaacggaccttgttggggaccctgag |
| | atattaaccgtggccttgtcctgcaggatgtgatacacatagaactaaagattgaaggaaagtgtgggaggggtgaagccaaa |
| | ggaaggtagcccatctgagtagaaagttctgtgcagggcagaggaactctggtgtggacccagagattcaacctactccaaga |
| | ttcaaccttggcctcgccctgcagGCCCTTCTGTGGTGGGCCTTGTCTCCATTCCAGGGATGGGCAAGTTGCTGGCTGAGGCC |
| | CCTCTGGTCCTGGAACCTGAGAAGCAGATGCTTCTGCATGAGACACACCAGGGCTTGCTGCAGGACGGCTCCCCCATCCTGCT |
| | CTCAGATGTGATCTCTGCCTTTCTGAGCAACAACGACACCCAAAACCTCAGCTCCCCAGTTACCTTCACCTTCTCCCACCGTg |
| | tgagtgctggtggagttggttggtgggtgaatagtctgagtccgggcatagccttgctgctcagctcagccctggggctcagg |
| | ggtctctgttatgggtacattatctccccaggaaagtcagtccttcccaagccggctttgggtaagcatttctgagcatccga |
| | cccaccagctcacacctgtgttcttttttttattgagacagagtctcgctgtgtcagccaggctggagtgcagtggcacgatc |
| | tcagctcactgcagtctccgcctcccaggttcgagcgattctcctgcctcagcctccctaatagctgggactacaggtgtgtg |
| | ctaccacgcccagctaatttttgtattttttttttttttttttttttttagtagagatggggtttcaccatgttggccaggct |
| | ggtctcaaactcctgacctcaagtgatccacctgcttcagcctcccaaagtgctgggattacaggcgtgagccaccatgccca |
| | gcccacacctgtgttctgttcctgcagTCAGTGATCCCGAGACAGAAGGTGCTCTGTGTCTTCTGGGAGCATGGCCAGAATGG |
| | ATGTGGTCACTGGGCCACCACAGGCTGCAGCACAATAGGCACCAGAGACACCAGCACCATCTGCCGTTGCACCCACCTGAGCA |
| | GCTTTGCCGTCCTCATGGCCCACTACGATGTGCAGgtgagacccttaggaggggatgcactctgcatttattgccgtgtaaca |
| | aatccccagagacgtagcagccttttaaaaaaatatatgcattatctcacagctctgggtcagcaagctggcatagcaagatg |
| | gcttttctgctcagggtcttacaagactgaaatcatcatgtcaccccacccccagggctgccatttcatctgagacttgaggt |
| | ccttctccaagctccctggttgctggcagaattcagtttcttgtgattgtaggactgaagtctcactttcttagctgtcagga |
| | aggcatcacattcagttcctagaggccactcatatcccttctcacatgaccctctggcaaacttctagcattcctatctggag |
| | gacaaggaaaaaaatttcctcacgcagaatccctcttaagctttgtattagtcgaggttctctagagggacagaactaatgga |
| | atagatagatagatagatagatagatagatagatagatagatagatagataatttctgcctgcttatattctagccacgctgg |
| | cagctgattagatggtgcccacccagattaagagtggatctgcctttcccaacccactgactcaaatatatacatatatagag |
| | atatatagagagagatctatatatatagatagatatagatatatataaaggggagtttattaggtattaactcacatgatcac |
| | aaggtcccacgataggccgtctgcaggctgaggagcaaggagagccagtccgagtcccagaactgaagaacttggagtctgat |
| | gttcaagggcaggaagcatccagcacaggagaaagatgtaggctgggaggctaggccagtctctcttttcacatttttctgcc |
| | tgcttatattctagccacgctggcagctgattagacagtgcccacccagattaagagtggatctgcctttcccagtccactga |
| | ctcaaatgttaatttcctttggcaacaccctgatagacacacccaggatcaatactttgtatccttcaatccaatcaagttga |
| | cacccagtattaaccgtcacaagttttgaatctgattctccaggaagagcccagagcttataaagactcatctgattaggtct |
| | ggcccacccagggtaaactctgccttttttttttttttagacagggtctctgtcacccaggctggagtgcagtggcacagtca |
| | tagctcactgcagccttgaccttctgggttcaagggatcctcctgcctcagcctcctgagtagctgggactacaggcatgcac |
| | caccatgcccggctaatttttgtatttttactagagatggggtttcaccaagttggccaggctggtctcgaactccttacctc |
| | aagtaatccacccgcctcggcctcccaaagtgctggtattacaggcaggaaccaccacacctcgcccacctgactttcttatc |
| | tcatcacttagagtagatccaagggacatcatcatcatatcctgcgggagaaagggccaaaccacccttttgttttccatgac |
| | aggaacatcttatcaatatcctcccaggcagcaagccataccgcccagcccctcccgcccagacctgtaattaccccagcctg |
| | taagcggcagtgggttctggcacgaagctagctcccccctccacaagtctcctgctggacataaacctgcattgctgtagagc |
| | tgccaactctctgtctttctttaactctcactttcccttcaaaacctaacgggcccacacctggaatctctgtgctttgggag |
| | gctgaaacgggaggatcacttgagcccaggaattcgagaccagcctgtgcaacacagaaagaccctgtctctacaaaaataaa |
| | aaaaaaattaggacgggtgtggtggctcacgactgtaatcccagcactttgggaggccgaggcgggtggatcatgaggtcagg |
| | agttcaagaccagcctggccaacatggtgtgaccctgtctctactaaaagtataaaaattagcctggcatggaggcacgtgcc |
| | tgtaatcccaactacttgggaggctgagacacgagaatcgcttgaaccgaggagacagaggctgcagtgagcagagatcgcac |
| | cactgcactccagcctgggcgacagagcaagactgtcaaaaaaaaaaattagcagggcatggtgccatacatgactataatgc |
| | acacccgttgtcacctacatgactacgcctgtattcctagccacccatgaggctcgggtgacaccttcgcaaattaaattaaa |
| | ttggtgacatcttcacaaatttacctggctccctcattcttgaatgtgctatgccaaagtaaaaattttaaatggtggtgggg |
| | tcattaccccagagcaggaaagcatcactgtgaactcttcttttccgggtcccagGAGGAGGATCCCGTGCTGACTGTCATCA |
| | CCTACATGGGGCTGAGCGTCTCTCTGCTGTGCCTCCTCCTGGCGGCCCTCACTTTTCTCCTGTGTAAAGCCATCCAGAACACC |
| | AGCACCTCACTGCATCTGCAGCTCTCGCTCTGCCTCTTCCTGGCCCACCTCCTCTTCCTCGTGGCAATTGATCAAACCGGACA |
| | CAAGgtattgacagcggtgttccagagggctcccttccttgacgcaggacacgctggctcatggaatgatgcggcatttgatt |
| | ccttaatataatagcccggctgggcgtggtggctcatgcctgtaatcccagcactttgggagactgaggtgggcggatcacct |
| | caggtcgggagtttgagaccagcctgaccaacatggagaaaccccgtctctactaaaaatacaaaattagccaggcatggtgg |
| | cgcatacctgtaatcccagttactcaggaggctgaggcaagagaatctcttgaacccaggaggtggaggttgtggtaagccga |
| | gatcgcaccattgcatgccagcctgggcaacaagagtgaaattccgtctcaaaaaaaaaaaaaaaaaaatatatatatatata |
| | tatatgtgtgtgtgtgtgtgtgtgtatacgtatatatacacgtatatatatataggcccttgagtcctggggaatattagaag |
| | agaacaccaccaatttcccattgcctgactcaactacacagcaaatattctctataatgccatcaaatatctcagaattctcc |
| | ccaacaacatggtacaggagaagccatgggccacactcaaatctgatcgccgctctgggcgtcttttacttctccgccagGTG |
| | CTGTGCTCCATCATCGCCGGTACCTTGCACTATCTCTACCTGGCCACCTTGACCTGGATGCTGCTGGAGGCCCTGTACCTCTT |
| | CCTCACTGCACGGAACCTGACGGTGGTCAACTACTCAAGCATCAACAGATTCATGAAGAAGCTCATGTTCCCTGTGGGCTACG |
| | GAGTCCCAGCTGTGACAGTGGCCATTTCTGCAGCCTCCAGGCCTCACCTTTATGGAACACCTTCCCGgttagtgcaaattctc |
| | acaatccttatcttctccggcataaccatggccattgctagaacctagatagcatttttatgacacctgcttttaattagatc |
| | agtagggattttaggctctattggttggattacactgcagctccttattcttagctattgagtttatttcaattacatttcaa |
| | gctaggcattgtagaagagggttttcggaaattttctgaatgaaataaagggcaagcctttggaaagcacgaataggacaaag |
| | acactatgtcataatccctagggcatctttctgctagaagattataataattaaggagggtcaggctcagtggctcgcacctg |
| | gaatccctgtgctttgggaggctgacacaggaggatcacttgagcccaggatcttgagaccagcctgggcaacacagcaagac |
| | cccttctttacaaaatttaaaaaattagcagggcctactggcacacacctgtagtcctagctactcaggaggctgaggtggga |
| | ggatcgcttgagcccaggagttcgaagttgcagtgagctatgattgcaccactgcactccagcctgagcaagagagaaagacc |
| | ttgtctctaaaaaataataataatagttagggataatgcatttaaatctgtctcactgaaatagtaagaatgaaatcaaagtg |
| | ctcaagacagcattttttaaaaatctaacctactttatctaaatcttttaggatactccaatttttcatttgaaaaagtagac |
| | ctgatttgaataactaaaataattgatattgtcatagttaagatttatgagatgcttactttgtgccaggtacttttctagct |
| | agtttgtttgtttgtttgtttgccaattgcactggttgaaacctccaggacactgttgactagaacagcagacaattttgtct |
| | tgctcctgtacttagggagaaagcattgactctgtcctcattaatgtatgatgttagctgaggatttttcatagatacccttt |
| | ttcaggttaaagaaattcttttctattcctcatgtgttcggtattttgatgaagaaagagtgtttgattttgtcaaatgcttt |
| | tccgaagcatataatttttaattatatggggtaaaaattatatgatcacataattagataatcatataattatataattatat |
| | tagattattttatatgtaatttttatataaatatatatctatgtaatttatatattaatattatataactataaataattata |
| | taacatagctataaataattatgtaactatgaataattacataacatagctataaataattatgtaactatgaataattacat |
| | aacatagctataaataattatgtaactatgaataattacataacatagctataaataattatgtaactatgaataattacata |
| | acatagctataaataattatgtaactatgaataattacataacatagctataaataattatgtaactatgaataattacataa |
| | catagctataaataattatgtaactatgaataattacataacatagctataaataattatgtaactatgaatacataacatag |
| | ctataaataattatgtaactatgaataattacataacacagctataaataattatgtaactatgaataattacataacatagc |
| | tataaataattatgtaactatgaataattacataacatagctataaataattatgtaactataaataattacataacatagct |
| | ataaataattatataactataaataattacataacatagctataaataattatgtaactataaataattatataacatagcta |
| | taaataattatataactataaataattacataacataactataaataattatataaccataaataattatattacataactat |
| | aaataattatattacataactaaataattatattacataactaattatattacataactataaataattatattacataacta |
| | taaataattatataactataaataattatattatataactataaataatatgatcacattaaattgtataatcatatattatc |
| | atatcattataattatattatatatgtatatatcatatataattacatataattatataagcataattatatgcttccttaat |
| | tctttattttattaatacacatattaatagaataaaggattaaaattatatgatcacattataattatatggttatataatta |
| | tatggttatatatgattaattatttacctttgagtcctggggaatattatatggttatatatggctaattatatggttatata |
| | attataatgtgatcttataattatatgattacataattatataactataatgtgatcatataactttaatcctttattctatt |
| | aatatgtgtattacattgatggatgattagatgttaaactagccttacattcctgggataattaccacctggtcagggtatat |
| | aatccttttaatatgttgcaggacttggtcggctagtatttttttgaggatttttgcattcattttcataagggattttggtc |
| | tgcagttttcttttcttgtgatgtctatttctgggtttggtgtcagagtaacactggcctcgtagaagatttggaaagtgctt |
| | cctctttttccgtttttggttttgtgaactattaacactaatcctccaaaagttaaccatagagctaccatatgacccaggta |
| | tatacccaaaacaaatgaaaacgatgtccacaaaaaaacttgtacatgatgcagccataaagaataaggagatcggccgggtg |
| | cggtggctcacacctggaatcccagcactttgggaggccgaggcgagcagatcacaaggtcaagagattgagaccatcctggc |
| | caacatggtgaaaccccgtctctactaaaaatacaaaagttagctgggcgtggtcgtgggtgcctgtaatcccagctacttgg |
| | gaggctgaggcacaagaatcgcttgaacctgggaggtggagtttgcagtgagccaagatggctccattgcacttcagcctggc |
| | cacagagcgagactccatctcaaaaaaataaaataacaagatcatgtcctttgcagcaatatggatggagctggaggccatta |
| | ttctaagtgaattaatgcaggaacagaaaatcaaataccgcatgttctcacttataagtaggagctaaacattgggtacccat |
| | ggacacaaacaaggaaacttcagacaccgaagcctgcttgagggtggagagtgggaggagggcgaggataaataaacgaccta |
| | tagagtactgtgctgatgaccagggtgacgaaataatctgtacaccaaaccgccgcaacatgcattttacctacataacaaac |
| | ctacaaaatgtacctctgaacctaaaataaacgtttagaaaaatgttgtacatgaatgctcatagaagcagtatttagaatag |
| | tcaaaaattggaaacaatgcctatcactgatgaatgaataaacaaaacgtaatatatccacaatgaaatgttattcagccata |
| | aaaaggaacgttaagagaccattcagtcattatttatttatttagaaacactctgtcacccaggctggagtgcagtggtgcca |
| | tcttggctcactgcaacctctgctctctgcttcctgggttcaagcaattctcatgcctcagcctcccgagtagctgagattac |
| | aggcatgcgccaccacacgcagctcattttttgtgtgtttttagtagagaaaggatttcaccacactgggcaagctggtctca |
| | aactcctgatatcaaatgatccacccgcctcggctcccaaagtgctgggattacaggtgtgaaccactgcacctggcctcatt |
| | cagccattttaaaatgctgcaatataggtcttgctctgttgcccaggctggagtgcagtagtgcaatcacagctcactgcagt |
| | ctcgacctcctggactcaagcgatcctcccacctcagtctcccaagtaactgggaatacaggtgtgagccaccacacccggct |
| | atttttttattttttgtagaaatggggtcttgctatgttgctcaggctggtctcaagctcttggtctcaagcaatcctcccac |
| | ctcggcctcccaaagtcctggtgtgagccactgtgcctggcacaaccttccatttttgtttcattgtttgaatgtgtctgatt |
| | gtgtgtatcttccttgagttgaaattttggggatctaagaccgtgttcattttctccaccactgtatcttcagagccaaccac |
| | agaccctatcactaaagaaacacttaatattgttgacgtaggaaggaagtgaggaagtggtgaggagggatggaatagattta |
| | cataactgttccatatctccaaaaactgatgcattttttttaatctctcttttttcagCTGCTGGCTCCAACCAGAAAAGGGA |
| | TTTATATGGGGCTTCCTTGGACCTGTCTGCGCCATCTTCTCTgtgagtgacatcatctgagcatcctcgctgccccgctgagg |
| | gtcattagaaaaacaagataataaagatggtgacaagagcaggaatgtcccctgggttgtcatgggttgggttaggtttccta |
| | agaccactcttgaatcttcaaactcaacactgttactcaatatttaagaaaaagacttacgtgtataattcatattagacagg |
| | ctaggatgaagagataatgggcttcaggcccggtgtggtggctcacgcctgtaatcccagcactttgggaggccgaggcaggc |
| | agatcacttgaggtcaggagttcaagaccagcctggccaacatggtgaaaccctgtctctactaaaaatacaaaaattagcca |
| | ggcgtggtggtgggtgcctgtactcgggaggctgagacaggagaatcgctcgaacccgggaggcagaggtggcagtgagctga |
| | gattgcaccgccacactccagcctgggcaacagagcaagaccctgtctgaaaaaaaaaaaaaaagagataacaggtttcaaaa |
| | tctattcaacaaatcttctgtggttttctgaagGTGAATTTAGTTCTCTTTCTGGTGACTCTCTGGATTTTGAAAAACAGACT |
| | CTCCTCCCTCAATAGTGAAGTGTCCACCCTCCGGAACACAAGgtaagatggagaagggggtgataaccacacagacatgtatg |
| | gttcccaagagccaagccatgtgcctcagcagccactgaggtcccttctcagggtcttttgaagggacccagaaaaaacagac |
| | gcagccatccttctatggcagccatcacagattcagtggtatcctgttgagaccccatgcaacatgttttgtccatgtctaat |
| | ctcagtttgtgaatcccagagcaacaggaagaatcttcattgactggtaaatggtatatatacaaaataggatacgattcgat |
| | cacaaaacagaatgaaaccatgtcatttgcagcaacatggatgaaactggaggtcactctcttaagtgaaataagccaggcac |
| | aaaaagacaaataccacaggttgtcacttacatagagaggagccgaagaagctgatcttgtggaggcagaaagtagaatgata |
| | aaagttagccgggcatggtggcaggcacctgtagtcccagctactcgggaggctgaggcaggagaaccgcttgaacccaggag |
| | gcggaggttgcggtgagccgagatcacaccactgcactccagcctgggcaatagagtgagactctgcctcaaaaactaaaaat |
| | aaaaataaagtagaatgatagattccagagtctggaaagggtatgtgagtgggcaaggcgatgaagagaggttggttaatggg |
| | tacaacatacagtcagatagaaggaataagttataatattggatggcagagtagggtgactctagttcacaatatgttgtata |
| | tttcaaaatagctagaggggagaatttgaagtgttcccaacacatacaaatggtagacacgcgaagtgacggataccccaaat |
| | actctgacttaatcatgacacactctatgcatgttaacaaaatatcacatatgtccccacaaagatgtacaaacattacgaat |
| | caataaactaaaaggttaaaaacttctttggacttttgtcagcagtcctataacaaaagcacaaagaaggccgggcatggtgg |
| | ctcatgcctgtaatcccagcactttgggaagctgaggggcgggggcagatcacaaggtcaggagttcgagaccagcctggcca |
| | aagagaccagcctgaccaacgtggtgaaaccccgtctctactaaaaatacaaaaattagctgggcgtggtggcgggtgcctgt |
| | aatcccagctactccagaggctgaggcaggagaattgtttgaacctgagaggcagaggttgcagtgagccgagattgcgccac |
| | tgcactccagcctaggtgacagagcaagactccaactcaaaaaaaaaagtgcaaagaacacgtacattttcttgcctgttccc |
| | acagcccagtctgcaccttcataccgtaccctcagttcaggctcctcatcacttctcatgcaatcctccctctgggaaaatcc |
| | taatcaacattaaatcctttacgggggccaggcatggtggctcacgcctgtaatcctaacactttgggaggccgaggtaggcg |
| | gatcacttgaggtcatgagttcaagaccagcctggccaacatggtgaaaccccatctctactaaaaatacaaaaattagccag |
| | gcatggtggtgggcgtctataatcccagctactcaggaggctgaggtgggagagtcacttcacctgggaggcagaggttgcag |
| | tgagccaagatcacgccactgcactccagcctgggtgacagagggagattccatctcaaaaaaataaataaataaaaataaaa |
| | ataattcctctacagggtttgagcagtttgttgttccaggcacccttgtgcaaaattaatgaactttcttctagattcctatc |
| | attatcctttccaaattttgtgtgtcatgcctggttgtattggtgatttgaagagactgtgtagttcatgaacacagaaatct |
| | ctttgcatattcaaaaataatcctttttttttttttgagacagagtctcgctctgtcactcccaggctggagtgcagtggctc |
| | tatctcgactcactgcaacctccgcctcctgagttcaagcaattctactgccccagcctcctgagtagctgggattacaggcg |
| | cccccccgaccacacctggctaatttttgtatttttttagtagagaccaggtttcaccatgttggtcgggctggtctcgaact |
| | cctgacctcaggtgatctgcctgccttggccccccaaagtgctgggattacaggcatgagccaccgtggccaaccccaggtgg |
| | catttctactgtgggctatagttgacagtaaatatctaagctgaccacagcccagtctgagaaatcatctacaggggagggca |
| | ggggactccatgggcacagatgaaatagccttgattctctttctctgggtgcatcaccagcctcctttcttactcaatccagG |
| | ATGCTGGCATTTAAAGCGACAGCTCAGCTGTTCATCCTGGGCTGCACGTGGTGTCTGGGCATCTTGCAGGTGGGTCCGGCTGC |
| | CCGGGTCATGGCCTACCTCTTCACCATCATCAACAGCCTGCAGGGTGTCTTCATCTTCCTGGTGTACTGCCTCCTCAGCCAGC |
| | AGgtaccactgcccagctcccacccaggactcttcctgtcctcactgctctctgtgagctgacccagtacacactttgcctct |
| | gcagGTCCGGGAGCAATATGGGAAATGGTCCAAAGGGATCAGGAAATTGAAAACTGAGTCTGAGATGCACACACTCTCCAGCA |
| | GTGCTAAGGCTGACACCTCCAAACCCAGCACGgtaagatcacgcattgctccagagcacttcactaaccgacccacctgagga |
| | gcatgtgcctatcacacaaggaaacctgggaatacagcaggcaatgccctagaaaggctcgcatctgagtacgccttgactca |
| | ttaaccattagcaatgatctcagtttaaatgtttttttttaatcagtcatagcctgtcatcccagcatcactgtcatcccagc |
| | atttgggaggcctaggcaagaggatcacctgaggccaggagtgcaagatgaccctgggcaacatagcaagatcccatctctac |
| | aaaaaataaaaataaaaattagccagacatggtggcatacacctgtagtcccagctactcctcgggaggctgaggtgggacaa |
| | tcacttgagcccagtaggttgaggctgtagtgagccatgatcatgccactgcactccagcccaggcaacagagtgagacccta |
| | tcccaaaaattaaaaaaaaaaaaaaaatccttggctcttactccttagggaactgtgcttagtttgactctcacacacaaaca |
| | ctcctattggacacctactatgtgccatgcatggttaactaaatgagcaggaattgatctgaagcagtgatgcacattctacc |
| | tagaagcatttaccctatccctttcctctacatacaagtctatctcacagtcacttggcaccaccacccaataaggcaagcat |
| | gggaggaaatgctgtacatatttatgaattaaacagaaataagtgttaactgggatttaggctttcttaacaagtctgcctct |
| | accaactaccacttagtagtgggtagtgggcagtgcctctactcagcaccctctacccactgcaaaactcagaaagaaaagca |
| | gtaatggggccagcgatggtggctcatgcctgtaatcccagcactttaggaggctgaggcaggacaactgcttgaggccaaga |
| | gttcaagactagcctgagcaacccagtgacacctcgtccctataaagcatttaaaaattagctgagggtggttgtgtgcatct |
| | gtagtcccagcaactcaggaagctgaggtgagaggatcacttgagcccaggagtttgaggctgcagtgagctatgattgtgcc |
| | actgcatttcagcctggatgacagcaagacctcttctctagaaaataatacaaataacaaaattaaataaataaagttttaaa |
| | aacacactgggcaacctgggaatcagcgggtgatgccctaagagggttcacatttgagcgactccttgactcattaaccatta |
| | gcaatgatctcagtttaaatgcttttttataaatcagccaggaacagtggttcaagcttgtaatgccagcactctgggaggcc |
| | aaagcaggaagatcacttgagcccgggacaacttggggatcacttgaagccatattatcctatgcctcaatggcatgggataa |
| | ttcatgacattgatgactaataaccaacatttattaatgtagacatacaaagttctgttgtatttttctcacctcttcttcca |
| | tcttctccagattatatgtgtaattctaaccccatttcactgatgagcaaagtaagactacttggatttgtgtcaaatgtaca |
| | gagtctcggccaggcacagtgactcatgcctgtaatcccagcactttgggaggccgaggcaggtggatcacaaagtcaggaga |
| | tcgagaccatcctggctaacacggtgaaaccccatctctactaaaaatacaaaaaaaaaaaaaaaatagccaggcgtggtggt |
| | gggcgactgtagtcccagctactcgggaggctgtggcaggagaatggtgtgaacccaggaggcagagctggcagtgagccgag |
| | atcgcgccaatgcactccagcctgggcaacagaacgagactctgtctcaaaaaaaaaaaaaaaaaaaaaagaaatgtacagag |
| | tctcttcagccagtgagatttaggcctcttgagtcttgcttcacatttaaagagcccattcagggccaggcaccgtggctcat |
| | acctataatcttagcacttcaggaggccaaggcaagcaaatcacctgaggccaggagttcgagaccagcctggccaacatggc |
| | aaaactccatctttactaaaaatacataaattagccaggcatggtggtgcacacctataatcccagctactcaggaggctgag |
| | gcacaagaattgcttgagccggggaggtaaaagttgtggtgagccaagattgccccatgccactgtactccaccctgggcaac |
| | acagcaatactctgccaaaaaaaaaaaaaaaaaaaacagcctattcagcctgggcaatgtaacgagaccccatctctataaaa |
| | aagttttaaaaattcgctgggcgtggtggcacatgcctgtagtcccagctacttgggaggcgagaggatcgcttgagcccagg |
| | agttggagaccaacctgtgcaacatggcaaaatcccatctctacacaaaatataaaaactagctgggcatggtggtgcatgcc |
| | tataatcccagctgcttgggaggctgaggcaggaggatcacttgagcccaggagttcaaggccacggtgagctacgattatgc |
| | cactgcactacagcctgggtgacagagcaagaccccttgtgtaaagtaaatacataaataataaattttttaaagtcttgaaa |
| | agaagcccaccatgctgccaggcactttttccacatttgcatttctctcattacctttttccttttttatttttctttttttt |
| | ttttttttgagacggagtttcgctcttattacccaggctggagtacaatggtgctatctcagctcaccgcaacctccgcctcc |
| | tgggttaaagcgattctcctgcctccacctcccaagtagctgggattacaggcatgtgccaccaagcctggctaattttgtat |
| | ttttactagagacgggatttctccatgttggtcaggttggtctcaaattcccgacctcaggtgatctgcccgcctcagcctcc |
| | caaagtactaggattacaagcgtgagccaccacacctggcctcattacctttttcatccaacctgcacaatccctgtttccaa |
| | cacaggatcttctatttccttttctgttctcactaaatgccctcaccacctatgtcacacacacacacacacacaaatccaag |
| | tcatcatgatgacacattgtaaactccatgctctcaatcatacaaccaaacatgaccatccaccttccacattcccatattat |
| | tgactgtccatcctctttttttttttttagacagagtctcactcgctctgtcacccaggctggagtgcagtgacaccatctgg |
| | gctcactgcaaactctgcctccagggttcaagtgattctcgtgcctcagcctcccgagtagctaggattacaggcacccgcca |
| | ccacgcctggctaatttttgtatttttagtagagacagggtttcaccatgttggccaggctagtcttgaactcctggcctcaa |
| | gccatctgcccgccttggcctcccaaagtgctgggattacagacgtgagccactgcgcccagtccatccctgtctttgactct |
| | cctacagccttcatactttataaagaccttgatgattacattgggttcaccaagacaatccagggcaattttcccatctcaag |
| | atccttttcataaccgtatctacaaagtctctttctccatgtcaggtagcatttacaggttctggggattagggatgtgaacc |
| | tctttggggggtcattattctgtctctcatacccatatagtttgggtttacctattgtagaatcatcaaaacctgcctcatat |
| | tgtagatatttgcataaatatttatcaccctttcaccccacacagccactagaaatgatatatacacattgaagaaaggaaac |
| | atgtacttttgtgtagggaattagagttaggtcatcagatagccaacaaaggcttctgaaatttaaggtgtgtgatcagatat |
| | ttctaccagctactgacaaaggaccaagaaaatccattgcattacttaatgtcattttatcttctactgtttcctaaaacttg |
| | ttatctctggtgtttgaaaatgtatgtgccttggccggacacgctggctcacacctgtaatcccaacactttgggaggccaag |
| | gcgggcagagcacttgaggtcaggagttcgagaccagcctggccaacatggcaagaccccacctctactaaaaacacaaaaac |
| | tagccaggcatggtggtggatgcttgtaattccagctacttgggaggctgaggcacaagaatcactcgaacccaggaggcaga |
| | ggttgcagtaaagccaagatcgagccactgcactccagcctgggtgacagagtgagactatgtctcaaaaaaaaaaaaaaaaa |
| | gaaaagaaaagaaaagaaaatgtgtgtcttgctcttattctgcagtctctgccatggctttctgcagtcttgtttcataaaaa |
| | caaaatgtcttgtgatcactactgaataatctcctgaagcacaatatttattaatctcttgagcacatattaattatgcatta |
| | aatactaactatataagcaagtgaagctgaccatctcgaaccatagaaattacactgagtttcctatgaacctaggaataaat |
| | aactcatattggagatggaaaagatatggaaaaaaggaattcaggctgggcgcagtggctcacgcttgtaatcccagcacttt |
| | gggaggccgaggcaggcagatcactttagctcaggacttcaagaccagcctgggtaacatggcaaaattccgtttctacaaaa |
| | agtataaaaattagccaggcatggtggtgcatgcctgtagtcccagctaccgggaggctaagattggagtattgcttgagccc |
| | aggaggtcaagtatccagtgagctatgatcccatcactgcactccagcctgggcaacaaagcaagacccctgtctcaggggaa |
| | aaaaaaggaattcagtgctttttcctttaaatcttatgtggttaacaatagataacacttctaacatttcattaatttcccac |
| | ctggcttcttttatgactccacatttaaagcaacattacacaaaccctggagctccgaggtcaagacatacatttggaaacag |
| | atagatctttttaaatgttttttaattgatccataatagacgtacatagtttctaggtacatgtgatcatttgctacactcat |
| | ataagcaaatcagggtaactaagatatctatcaccttaaatacttatcttttctttgtactaataacattcaaattattctct |
| | tctagctagtttgaaatgtatggtagattaatgttaactataatcaccctacttcagatacaacttttttttttttttttgag |
| | agggagttttactctgtcgcccagactggagtacagtggcaccatcttggctcactgtgacccccacctcccagattcaagcg |
| | attctcctgcctcagcctcccgagtagctgggactacaggcgcctgccaccaagcccagctaattgattattattattactac |
| | tgtattctagtagagacagggtttcaccatgttggccaggctggtctcaaactcctgacctcaagtgatctgcccgcctcggc |
| | ctcccaaagtgctgggattacaggtgtgagtcaccacacctggcccagatagatactttactgcaacacatttacttcctctt |
| | aactttggtaacttggggtttaggaggcatacaaagatggggcttctgagaaagaacaggatggagcaaaatgaaagctgata |
| | tggccttttgatgttgccctgacaaatacatatggtaagaaaaccttatgattaaagacttcttggccaggcatggtggctca |
| | cacctagaattccagcatttttgggaggttgagatgggaggatcacttgagcccagaagttcaacagcagcttgggctataca |
| | gtgagaccccatctctacaaaaaattaaaattaaaaaatgagacaggagtggtggcacatgcctgtagttgcagctactcggg |
| | aggctgaggcaggaggatcacttgaacctgggagttggaggttgcagtgagcagagatggtgccactgcactccagactgggt |
| | gacagagcaaggccctgtctctaaaaagaaaaaaaaaaaaaagacttcttaaatttcttttttttaataaattttattgtgta |
| | tacacacaattgaggtttaacccatgaagttatgggatacatatagataataaaatggttatctatatgtgaagcaaattaat |
| | atatctatcatctcacatagttacttatttttgtggcacaagcagctataatctacttatttaactaatttcttatttctttt |
| | tcaaaatggaaattataggttttctttctatagtcatatatatttctaaatatatatatatttctaaatatatagatattaaa |
| | tatatatatttctaaatatatagatattaaatatctatatatttctaaatatatagatattaaatatctatatatttctaaat |
| | atctatatatttctaaatatctatacttctaaatatctatatttctaaatatctatatatttctaaatatctatatttctaaa |
| | tatctatatatttctaaatatctatatatttctaaatatctatatatttctaaatatatatatttagaaatatctatgtattt |
| | ctaaatatctatatatttctaaatatctatatacttctaaatatatctatgtttctaaatatatttagaaatatatatatatt |
| | tcaggccagctcagtggctcacacctgtaatcccagcactttgggaggctgaggcaggcagatcacctgaggtcaggagttca |
| | aggccagcctggcctacgtggtgaaaccctgtctctactaaaaatacaaaattagccgggtgtggtggcacatgcctctaatc |
| | ctggctactctggaggctgagacaggagaattgcttgaacctgggaggcgggggttacagtgagtcgaggctgcgccattgca |
| | ctccagcccaggcaacaagagtgaaactctgtctcaacaacaaaatacatacatatatatatatttcagGTTAACTAGAAAAA |
| | TCTTCTGAATAAGATCTTCCCTCTTTGCCCGTGGAAAATCTGAACAATCTTTGAGCCATCTAGAGGGGAAAGAAAAGACTTTG |
| | TTCTGTGTGTTTCAAGAAATTCACCATGTCAGCAATATGAAGGATGTTATGGAAGGCGTGCTAGGCATTCAATTCCTGCAGAA |
| | ACCGGAAATCTTCCATGCCCTGCAATGTGCTCATCAAACTCTCAGCATATGGACGGCCAGCTGTGGCCCATATCTTGGTCACT |
| | CTGAAGCACAATATTTATGAAGCTATAGAACGTTAAGACCTCTTTCACAGCCTCTCCTTCCTACAAAGACTCCTCCAAATCTT |
| | AAAATGAAGCAGGAAAACGAGCCTAAGAGGACTTTCATACCGACAACATCTGAAAGGACTAGAATGTTCACACCACGATCTGG |
| | ATTTCTTAATTTTTTGTTTTTGTTTTTGTTGTTCTCTAGTTCTACGGGTTTGATTATTTAGTCATGTGAAAAATATTGATTAC |
| | TCACACATAGATCAAGAGAGACACGGCTCCTGCCTTCATGGAGCTTTTAGGGGAAAATGAAGTGGCTCTTGCAGCTAGAGTTG |
| | ACTCAGAAGCCGAAATTCCTAGAAATCAGGTTTCTACTGCTAGGCAATTGAAGTATAAACTATTTTATAAACACTGTCTTCTT |
| | TCGTCTTCACACCAACATGCAGAAAAGTTTCTAATCTCAGATCGGGGATGTGCAACAAATTCCATTTCAAAGGAATGACCTGC |
| | AAAACTCCTAAATATTCCAAGCAAATGCCCTTAACCCTGTCTGTTATCTGCTTTCCTTGAACAGAAATTCTACATGACCATAA |
| | AACCTCGAAGATGGGTATGGCACAGTTCATGCCCTGTAATCCTAGCACTTTGGGAGGGTGAGGCAGGAGGATGGCTCAAGCCC |
| | AGGAGTTTGAGACCAGTGTGGGCAACAGAGTGAGAACCATCTCTACCCAAAAAAAAAATTAAAAATTAGCCAAGCATGGTGAT |
| | GATATAGGAGTTAAGGAGAAATCATTTAGGCAAATAGCAAGGGTAGGAAGTCCTCAGTAAGGTTTTCCATTTAATGAAAAGCA |
| | GCCCCCAAAATCATTTTCTTTTCTAACAAAGAACAGCCTGTAAAATCGAGCTGCAGACATAGACAAGCAAGCTGGAAGCTTCC |
| | ACGGGTGAATGCCGGCAGCTGTGCCAATAGGAAAAAGCTACCTAGACTAGGCATGTCCAAAATGGCGGCTCCAAGTTCCCTTC |
| | TCTTTGCCAGCCATGTGTACAGTAAAAAGCAGGCAACATAGTGTCAGCCAAAGCTCATTTGCATAATAAGATTAGGGTGGGGT |
| | GGCCAGCTCACATAGGGGTAGGCCCTAGGTAAATCAGACACCGCCTTCTCAAGCCTGTCTATAAAATCTGGTACACTATGACG |
| | AGGGTCAGATTTCCCATTCAGACGCCCCTCTCCCATGCAAGAGAAAGAGCTGTTCTCCTTTCTCTTTCTTTTGCCTATTAAAC |
| | CTCTGCTCCTGGCCAGGCACAGTGGCTCACGCCTATAATCCCAGCACTTTGGGAGGCTGAGGTGGTCAGATCACCTAAGGTCA |
| | GGAGTTCAAGACCAGCCTGGTCAACATGGTGAAATCTTGTCTCTAGTAAAAATACAAAAATATATGAAATCTCACATAGATGA |
| | TAATATTAAGTTCCAAAAGCAACTCAACCTGGTAGATTCTAATTTTTTTTGAGGCAGGGTCTTGCTTTGTCACCCATGCTGGA |
| | GTACAATGGCACAAACACTGCTCACTGCAGCCTCGACCTCCCAAGGCCTAAGCAATCCTCCTGCCTCAGTCCCCCTCCAGGTA |
| | TTTGAAACTACAGGTGTGTACCACCACACCGGGCTAATTTTTGTATTTTTTGTAGAGACGTGGGTCTCACTATGCTGCCCAGG |
| | CTCAGGTCTTAATCTCCTGAGCTCAGGCAATCCGCAGGCCTCAGCCTCCCTAAGTGCGGGGATTACAGGCTTGAGCCACTGCA |
| | CCTAGCCTCTATTTGTTTTACAAAAGAGAAATTGAGATCCTGAATGTTAAGTGACTTGCCTGAGGCCATCCCACTAACAGGAG |
| | CCAGGGTTAGGATTCAAACCCCATCCAACTGGTCCCAGAGCTGGAGCTTCTTGCACTGCCCTACACTACCTACCATCTCCATC |
| | CTCTGGGCACCTTTTTATAAGAACCAAAACATTACAGAGCATTGCTTTGTCAACTCAGCTGGGAACATTTCCCAGTGCAACTC |
| | ACATTTTTCACTGCTCTGTGCCTGTCCGTATAAGCTCAATGAGTATTGATTTAGGGGCTTTGGAGAACTTTGAATGCTACCCC |
| | CCAAGTAACCATTGTTGGCAACCTGGTACCTCTACTTTTAGCCATTTCTCCTTCTCTATAAATAGTGCAGAAGTAACCCACTT |
| | GGTAACAGGCATCCTTGCCAAGCCTCCACCACTAGGTCAGTGTAAGAATTAAAGAAAGAGGAAAGAAACACAAAAAGTGGCTT |
| | GATGGTTAAGACAGGTTTATTTTAGAGAAAACACACCTGAGAGGGGCTGCTGGCTGAATTAGGTTAGAGTCTTTTCTACAGAC |
| | TAAGAGTGTTTAAGGATTTAGGGTGGGAGAGTTTCTTAGAGGCTTGGACTGCTTCTGTGTTTTTTTTGTTGTGCTTATATGGG |
| | AGGGAGAGTGGTGTGTTTGCTTTTATACATTTTTCTGCAGCTGTAGGCATACCCCCCAAGTCTGCTTTTAGCTTCCCTATTTT |
| | AGTGCACCTGGAGGGAAAGGAATGTGCTTATTAAGGCCCACTGTTTTACTGGGGCCCATTGTATGAGGGTGAAGTTTGGCAGT |
| | TACCCAAGAGACTTTTCCTCCACCTTCCTCTGTGCCCGAGCTGTTTTATCTGCATTTTACTGTCTGCTTTTTTTGGCTGCTTA |
| | TAGTTTTTAAAAAAGTAATTTCCTTAAATCCAGAAGGCTAAAAATGAAGCTGAAACTTAAAGTGGCGGTGTTTGTCCAAAATA |
| | ACGGGGCTCCTGCTCTGCCAGTCAGTACCCTCAAGTCACTCCTGATCCTCAACCTCCATGCCTAAGGCTGGTTCAAGAGACCA |
| | CATAATATCTGCCTTTTATTACATACATGATGGGTGCATGGGATTCTGCGTGCCCTTTGCTTGATATAGACTGCTAAGGTGAG |
| | ATGGGGAATATCAGAGTCAGCTGCTGCTTGAGGAAGCAGAACACACAGCTGGAGGCTTGGAACATGTGGGTCCCTATGAGTGT |
| | AGAGCCCATATCCCCATAGAGTCTACCTAGAGCAGGGGTCGCCAAATGTTTTCTTAAAGAGCCTGATAGTGTATATGTTAGGC |
| | TTTGTGAGCCAGGTATTTACAGCAACTCAATTCTACCACTGTGGTATGAAAACAGCTATAGACAATCATAAATGAATGATCAT |
| | GGCTATGTTTTAATAAAACTTTACAGACACTGAACTTGAACTTCCATTGTGATATGAAAACAGCTATAGACAATCATAAATGA |
| | ATGATCATGGCTATGTTTTAATAAAACTTTATGGACACTGAGCTTGAACTTCATATATCAGTCATGTGACACAAAATATCATT |
| | CTTCTTTTTATTTGTTTTCACCCATTTGAAAAATGTAAAAACTATTCTTAGCTGTACAGAAACAGATGGTGGGTCAGATTTGA |
| | CTCACTGCCCATAGTTTCCAGACCATGATGTTCAGGTTCATGGAAGCACTTCACTCTACACTAATTTATTCATCCATTCATCA |
| | CATATGTGATGAGCACATACAGACACACCTCAGAGATATTGCAGGCTCCACTCCAGATCACCACAATAATGTGAATATTGCAA |
| | TAAAGCAAGTCATACACATTT |

Dual and Multiple gRNA Compositions and Uses Thereof

In some embodiments, a gRNA described herein (e.g., a gRNA of Tables 1-19) can be used in combination with one or more gRNA, e.g., for directing nucleases to one or more sites in a genome. In some embodiments, multiple gRNA described herein (e.g., two or more gRNA of Tables 1-19) can be used in combination, e.g., for directing nucleases to multiple sites in a genome. In some embodiments, a gRNA described herein (e.g., a gRNA of Tables 1-19) can be used in combination with a second gRNA, e.g., for directing nucleases to two sites in a genome. In some embodiments, a gRNA described herein (e.g., a gRNA of Tables 1-19) can be used in combination with a third gRNA, e.g., for directing nucleases to three sites in a genome. In some embodiments, a gRNA described herein (e.g., a gRNA of Tables 1-19) can be used in combination with a fourth gRNA, e.g., for directing nucleases to four sites in a genome. In some embodiments, a gRNA described herein (e.g., a gRNA of Tables 1-19) can be used in combination with a fifth gRNA, e.g., for directing nucleases to five sites in a genome. In some embodiments, a gRNA described herein (e.g., a gRNA of Tables 1-19) can be used in combination with a sixth gRNA, e.g., for directing nucleases to six sites in a genome. In some embodiments, a gRNA described herein (e.g., a gRNA of Tables 1-19) can be used in combination with a seventh gRNA, e.g., for directing nucleases to seven sites in a genome. In some embodiments, a gRNA described herein (e.g., a gRNA of Tables 1-19) can be used in combination with an eighth gRNA, e.g., for directing nucleases to eight sites in a genome. In some embodiments, a gRNA described herein (e.g., a gRNA of Tables 1-19) can be used in combination with a ninth gRNA, e.g., for directing nucleases to nine sites in a genome. In some embodiments, a gRNA described herein (e.g., a gRNA of Tables 1-19) can be used in combination with a tenth gRNA, e.g., for directing nucleases to ten sites in a genome. In some embodiments, a gRNA described herein (e.g., a gRNA of Tables 1-19) can be used in combination with more than tenth gRNA, e.g., for directing nucleases to more than ten sites in a genome.

For instance, in some embodiments it is desired to produce a hematopoietic cell that is deficient for a first lineage-specific cell-surface antigen (e.g., a lineage-specific cell-surface antigen, e.g., CD33, CLL-1, CD123, CD19, CD30, CD5, CD6, CD7, CD34, CD38, or BCMA) and a second lineage-specific cell-surface antigen (e.g., a lineage-specific cell-surface antigen, e.g., CD33, CLL-1, CD123, CD19, CD30, CD5, CD6, CD7, CD34, CD38, or BCMA), e.g., so that the cell can be resistant to two agents: an agent targeting the first lineage-specific cell-surface antigen and an agent targeting the second lineage-specific cell-surface antigen. In some embodiments, it is desirable to contact a cell with two or more different gRNAs that target different regions of a lineage-specific cell-surface antigen (e.g., a lineage-specific cell-surface antigen, e.g., CD33, CLL-1, CD123, CD19, CD30, CD5, CD6, CD7, CD34, CD38, or BCMA), in order to make two or more cuts and create a deletion between the two cut sites.

Accordingly, the disclosure provides various combinations of gRNAs and related CRISPR systems, as well as cells created by genome editing methods using such combinations of gRNAs and related CRISPR systems. In some embodiments, the first lineage-specific cell-surface antigen gRNA binds a different nuclease than the second gRNA. For example, in some embodiments, the first lineage-specific cell-surface antigen gRNA may bind Cas9 and the second gRNA may bind Cas12a, or vice versa.

Accordingly, the disclosure provides various combinations of gRNAs and related base editing systems, as well as cells created by genome editing methods using such combinations of gRNAs and related base editing systems.

In some embodiments, two or more (e.g., 3, 4, or more) gRNAs described herein are admixed. In some embodiments, each gRNA is in a separate container. In some embodiments, a kit described herein (e.g., a kit comprising one or more gRNAs according to Tables 1-19) also comprises a Cas9 molecule, or a nucleic acid encoding the Cas9 molecule.

In some embodiments, it is desirable to contact a cell with two or more different gRNAs that target different sites of CD33 (Siglec-3), CLL-1, CD123, CD327 (Siglec-6), and/or CD312 (EMR2), e.g., in order to make multiple chemical alteration to a nucleobase(s). In some embodiments, the first and second gRNAs are gRNAs according to Tables 1-19, or variants thereof.

In some embodiments, it is desirable to contact a cell with two or more different gRNAs that target different sites of CD33, e.g., in order to make multiple chemical alteration to a nucleobase(s). In some embodiments, the first and second gRNAs are gRNAs according to Tables 1-19, or variants thereof.

In some embodiments, it is desirable to contact a cell with two or more different gRNAs that target different sites of CLL-1, e.g., in order to make multiple chemical alteration to a nucleobase(s). In some embodiments, the first and second gRNAs are gRNAs according to Tables 1-19, or variants thereof.

In some embodiments, it is desirable to contact a cell with two or more different gRNAs that target different sites of CD123, e.g., in order to make multiple chemical alteration to a nucleobase(s). In some embodiments, the first and second gRNAs are gRNAs according to Tables 1-19, or variants thereof.

In some embodiments, the first gRNA is a CD33 gRNA described herein (e.g., a gRNA of Tables 1-5 or a variant thereof) and the second gRNA targets a lineage-specific cell-surface antigen chosen from: BCMA, CD19, CD20, CD30, ROR1, B7H6, B7H3, CD23, CD33, CD38, C-type lectin like molecule-1 (CLL-1), CS1, IL-5, L1-CAM, PSCA, PSMA, CD138, CD133, CD70, CD7, CD13, NKG2D, NKG2D ligand, CLEC12A, CD11, CD123, CD56, CD30, CD34, CD14, CD66b, CD41, CD61, CD62, CD235a, CD146, CD326, LMP2, CD22, CD52, CD10, CD3/TCR, CD79/BCR, and CD26. In certain embodiments, the second gRNA is a CLL-1 or CD123 gRNA.

In some embodiments, the first gRNA is a CD33 gRNA described herein (e.g., a gRNA according to Tables 1-5 or a variant thereof) and the second gRNA targets a lineage-specific cell-surface antigen associated with a specific type of cancer, such as, without limitation, CD20, CD22 (Non-Hodgkin's lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, biliary adenocarcinomas and ductal adenocarcinomas of the pancreas) as well as prostate specific membrane antigen.

In some embodiments, the first gRNA is a CD33 gRNA described herein (e.g., a gRNA according to Tables 1-5 or a variant thereof) and the second gRNA targets a lineage-specific cell-surface antigen chosen from: CD7, CD13, CD19, CD22, CD20, CD25, CD32, CD38, CD44, CD45, CD47, CD56, 96, CD117, CD123, CD135, CD174, CLL-1, folate receptor β, ILIRAP, MUC1, NKG2D/NKG2DL, TIM-3, or WT1.

In some embodiments, the first gRNA is a CD33 gRNA described herein (e.g., a gRNA according to Tables 1-5 or a variant thereof) and the second gRNA targets a lineage-specific cell-surface antigen chosen from: CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD851, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD14, CDwl45, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDwl98, CDwl99, CD200, CD201, CD202b, CD203c, CD204, CD205, CD206, CD207, CD208, CD209, CD210a, CDw210b, CD212, CD213al, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD272, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362 or CD363.

In some embodiments, the first gRNA is a CD33 gRNA described herein (e.g., a gRNA according to Tables 1-5 or a variant thereof) and the second gRNA targets a cell-surface antigen, e.g., chosen from: CD1a, CD1b, CD1c, CD1d, CD1e, CD2, CD3d, CD3e, CD3g, CD4, CD5, CD6, CD7, CD8a, CD8b, CD9, CD10, CD11a, CD11b, CD11c, CD11d, CD13, CD14, CD15, CD16a, CD16b, CD17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c,CD61, CD62E, CD62L, CD62P, CD63, CD64, CD65s, CD66a, CD66b, CD66c, CD66d, CD66e, CD66f, CD67, CD68, CD69, CD70, CD71, CD72, CD73, CD74, CD75, CD75s,CD77, CD79a, CD79b, CD80, CD81, CD82, CD83, CD84, CD85a, CD85b, CD85c, CD85d, CD85e, CD85f, CD85g, CD85h, CD85i, CD85j, CD85k, CD86, CD87, CD88, CD89, CD90, CD91, CD92, CD93, CD94, CD95, CD96, CD97, CD98, CD99, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD122, CD123, CD124, CD125, CD126, CD127, CD128a, CD128b, CD129, CD130, CD131, CD132, CD133,CD134, CD135, CD136, CD137, CD138, CD139,CD140a, CD140b, CD141, CD142, CD143, CD144, CD146, CD147, CD148, CD150, CD151, CD152, CD153, CD154, CD155, CD156a, CD156b, CD156c, CD157, CD158a, CD158b1, CD158b2, CD158c,CD158d, CD158e1, CD158e2, CD158f, CD158g,CD158h, CD158i, CD158j, CD158k, CD159a, CD159c, CD160, CD161, CD162, CD163, CD164, CD165,CD166, CD167a, CD167b, CD168, CD169, CD170, CD171, CD172a, CD172b, CD172g, CD173, CD174, CD175,CD175s, CD176, CD177, CD178, CD179a, CD179b, CD180, CD181, CD182, CD183, CD184, CD185, CD186, CD191, CD192, CD193, CD194, CD195, CD196, CD197, CD198, CD199, CD200, CD201, CD202b, CD203c, CD204, CD205, CD206, CD207, CD208, CD209, CD210a, CD210b, CD212, CD213al, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD238, CD239, CD240CE, CD240D, CD241, CD242, CD243, CD244, CD245,CD246, CD247, CD248, CD249, CD252, CD253, CD254, CD256, CD257, CD258, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300d, CD300e, CD300f, CD300g, CD301, CD302, CD303,CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD360, CD361, CD362, CD363, CD364, CD365, CD366, CD367, CD368, CD369, CD370, and CD371.

In some embodiments, the second gRNA is a gRNA disclosed in any of PCT Publication Nos. WO2017/066760, WO2019/046285, WO/2018/160768, or in Borot et al. PNAS (2019) 116 (24):11978-11987, each of which is incorporated herein by reference in its entirety.

In some embodiments, the first gRNA is a CD33 gRNA described herein (e.g., a gRNA according to Tables 1-5 or a variant thereof) and the second gRNA targets a lineage-specific cell-surface antigen chosen from: CD19; CD123; CD22; CD30; CD171; CS-1 (also referred to as CD2 subset 1, CRACC, SLAMF7, CD319, and 19A24); C-type lectin-like molecule-1 (CLECL1); epidermal growth factor receptor variant III (EGFRvIII); ganglioside G2 (CD2); ganglioside GD3 (aNeu5Ac(2-8)aNeu5Ac(2-3)bDGalp(1-4)bDGlep(1-1)Cer); TNF receptor family member B cell maturation (BCMA), Tn antigen ((Tn Ag) or (GalNAc.alpha.-Ser/Thr)); prostate-specific membrane antigen (PSMA); Receptor tyrosine kinase-like orphan receptor 1 (ROR1); Fms-Like tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor I receptor (IGF-I receptor), carbonic anhydrase IX (CAIX), Proteasome (Prosome, Macropain) Subunit, Beta Type 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDG1cp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex; locus K 9 (LY6K); Olfactory receptor 51E2 (OR51E2); TCR Gamma Alternate Reading Frame Protein (TARP); Wilms tumor protein (WTi); Cancer/testis antigen 1 (NY-ESO-1); Cancer/testis antigen 2 (LAGE-1a); Melanoma-associated antigen 1 (MAGE-A1), ETS translocation-variant gene 6, located on chromosome 12p (ETV6-AML); sperm protein 17 (SPA17); X Antigen Family, member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-1AP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxy esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2), lymphocyte antigen 75 (LY75); Glypican-3 (GPC3); Fc receptor-like 5 (FCRL5); and immunoglobulin lambda-like polypeptide 1 (IGLL1).

In some embodiments, the first gRNA is a CD33 gRNA described herein (e.g., a gRNA according to Tables 1-5 or a variant thereof) and the second gRNA targets a lineage-specific cell-surface antigen chosen from: CD11a, CD18, CD19, CD20, CD31, CD33, CD34, CD44, CD45, CD47, CD51, CD58, CD59, CD63, CD97, CD99, CD100, CD102, CD123, CD127, CD133, CD135, CD157, CD172b, CD217, CD300a, CD305, CD317, CD321, and CLL-1.

In some embodiments, the first gRNA is a CD33 gRNA described herein (e.g., a gRNA according to Tables 1-5 or a variant thereof) and the second gRNA targets a lineage-specific cell-surface antigen chosen from: CD123, CLL-1, CD38, CD135 (FLT3), CD56 (NCAM1), CD117 (c-KIT), FRβ (FOLR2), CD47, CD82, TNFRSF1B (CD120B), CD191, CD96, PTPRJ (CD148), CD70, LILRB2 (CD85D), CD25 (IL2Ralpha), CD44, CD96, NKG2D Ligand, CD45, CD7, CD15, CD19, CD20, CD22, CD37, and CD82.

In some embodiments, the first gRNA is a CD33 gRNA described herein (e.g., a gRNA according to Tables 1-5 or a variant thereof) and the second gRNA targets a lineage-specific cell-surface antigen chosen from: CD7, CD11a, CD15, CD18, CD19, CD20, CD22, CD25, CD31, CD34, CD37, CD38, CD44, CD45, CD47, CD51, CD56, CD58, CD59, CD63, CD70, CD82, CD85D, CD96, CD97, CD99, CD100, CD102, CD117, CD120B, CD123, CD127, CD133, CD135, CD148, CD157, CD172b, CD191, CD217, CD300a, CD305, CD317, CD321, CLL-1, FRβ (FOLR2), or NKG2D Ligand.

In some embodiments, the first gRNA is a CD33 gRNA described herein (e.g., a gRNA according to Tables 1-5 or a variant thereof) and the second gRNA targets CLL-1.

In some embodiments, the first gRNA is a CD33 gRNA described herein (e.g., a gRNA according to Tables 1-5 or a variant thereof) and the second gRNA targets CD123.

In some embodiments, the first gRNA is a CD33 gRNA described herein (e.g., a gRNA according to Tables 1-5 or a variant thereof) and the second gRNA comprises a sequence from Tables 1-19.

In some embodiments, the first gRNA is a CD33 gRNA comprising a targeting domain, wherein the targeting domain comprises a sequence of Tables 1-5, and the second gRNA comprises a targeting domain corresponding to a sequence of Tables 1-19.

In some embodiments, the first gRNA is a CD33 gRNA described herein (e.g., a gRNA according to Tables 1-s or a variant thereof) and the third, fourth, fifth, sixth, seventh, eight, nineth, tenth or more gRNA comprises a sequence from Tables 1-19.

In some embodiments, the first gRNA is a CD33 gRNA comprising a targeting domain, wherein the targeting domain comprises a sequence of Table 1-5, and the third, fourth, fifth, sixth, seventh, eight, nineth, tenth or more gRNA comprises a targeting domain corresponding to a sequence of Tables 1-19.

In some embodiments, the second gRNA is a gRNA disclosed in any of WO2017/066760, WO2019/046285, WO/2018/160768, or Borot et al. PNAS Jun. 11, 2019 116 (24) 11978-11987, each of which is incorporated herein by reference in its entirety.

### Cells Comprising Two or More Chemical Alteration to a Nucleobase

In some embodiments, an engineered cell described herein comprises two or more (e.g., 2, 3, 4, 5, 6, 7, 8, 9, 10, or more) chemical alterations to a nucleobase. In some embodiments, an engineered cell described herein comprises two or more mutations (e.g., 2, 3, 4, 5, 6, 7, 8, 9, 10, or more). In some embodiments, an engineered cell described herein comprises two or more (e.g., 2, 3, 4, 5, 6, 7, 8, 9, 10, or more) chemical alterations to a nucleobase in CD33 (Siglec-3), CLL-1, CD123, CD327 (Siglec-6), and/or CD312 (EMR2). In some embodiments, an engineered cell described herein comprises two or more (e.g., 2, 3, 4, 5, 6, 7, 8, 9, 10, or more) chemical alterations to a nucleobase, the first chemical alteration to a nucleobase being in CD33 and the second chemical alteration to a nucleobase being in a second lineage-specific cell-surface antigen. Such a cell can, in some embodiments, be resistant to two agents: an anti-CD33 agent and an agent targeting the second lineage-specific cell-surface antigen. In some embodiments, such a cell can be produced using two or more gRNAs described herein, e.g., a gRNA of Tables 1-19 and a second gRNA. In some embodiments, such a cell can be produced using two or more gRNAs described herein, e.g., a gRNA of Tables 1-19 and a second gRNA. In some embodiments, such a cell can be produced using two or more gRNAs described herein, e.g., a gRNA of Tables 1-19 and a second gRNA. In some embodiments, the cell can be produced using, e.g., a ZFN or TALEN. The disclosure also provides populations comprising cells described herein.

In some embodiments, the second chemical alteration to a nucleobase is at a gene encoding a lineage-specific cell-surface antigen, e.g., one listed in the preceding section. In some embodiments, the second mutation is at a site listed in Tables 1-19.

Typically, a mutation effected by the methods and compositions provided herein, e.g., a mutation in a target gene, such as, for example, CD33 (Siglec-3), CLL-1, CD123, CD327 (Siglec-6), and/or CD312 (EMR2) and/or any other target gene mentioned in this disclosure, results in a loss of function of a gene product encoded by the target gene, e.g., in the case of a mutation in the CD33 (Siglec-3), CLL-1, CD123, CD327 (Siglec-6), and/or CD312 (EMR2) gene, in a loss of function of a CD33 (Siglec-3), CLL-1, CD123, CD327 (Siglec-6), and/or CD312 (EMR2) protein. In some embodiments, the loss of function is a reduction in the level of expression of the gene product, e.g., reduction to a lower level of expression, or a complete abolishment of expression of the gene product. In some embodiments, the mutation results in the expression of a non-functional variant of the gene product. For example, in the case of the mutation generating a premature stop codon in the encoding sequence, a truncated gene product, or, in the case of the mutation generating a nonsense or missense mutation, a gene product characterized by an altered amino acid sequence, which renders the gene product non-functional. In some embodiments, the function of a gene product is binding or recognition of a binding partner. In some embodiments, the reduction in expression of the gene product, e.g., of CD33 (Siglec-3), CLL-1, CD123, CD327 (Siglec-6), and/or CD312 (EMR2), of the second lineage-specific cell-surface antigen, or both, is to less than or equal to 50%, less than or equal to 40%, less than or equal to 30%, less than or equal to 20%, less than or equal to 10%, less than or equal to 5%, less than or equal to 2%, or less than or equal to 1% of the level in a wild-type or non-engineered counterpart cell.

In some embodiments, at least 40%, at least 50%, at least 60%, at least 70%, at least 75%, at least 80%, at least 85%, at least 90%, or at least 95% of copies of CD33 (Siglec-3), CLL-1, CD123, CD327 (Siglec-6), and/or CD312 (EMR2) in the population of cells generated by the methods and/or using the compositions provided herein have a mutation. In some embodiments, at least at least 40%, at least 50%, at least 60%, at least 70%, at least 75%, at least 80%, at least 85%, at least 90%, or at least 95% of copies of the second lineage-specific cell-surface antigen in the population of cells have a mutation. In some embodiments, at least 40%, at least 50%, at least 60%, at least 70%, at least 75%, at least 80%, at least 85%, at least 90%, or at least 95% of copies of CD33 (Siglec-3), CLL-1, CD123, CD327 (Siglec-6), and/or CD312 (EMR2) and of the second lineage-specific cell-surface antigen in the population of cells have a mutation. In some embodiments, the population comprises one or more wild-type cells. In some embodiments, the population comprises one or more cells that comprise one wild-type copy of CD33 (Siglec-3), CLL-1, CD123, CD327 (Siglec-6), and/or CD312 (EMR2). In some embodiments, the population comprises one or more cells that comprise one wild-type copy of the second lineage-specific cell-surface antigen.

### Cells

Some aspects of this disclosure provide genetically engineered cells comprising a modification in their genome that results in a loss of expression of CD33 (Siglec-3), CLL-1, CD123, CD327 (Siglec-6), and/or CD312 (EMR2), or expression of a variant form of CD33 (Siglec-3), CLL-1, CD123, CD327 (Siglec-6), and/or CD312 (EMR2) that is not recognized by an immunotherapeutic agent targeting CD33 (Siglec-3), CLL-1, CD123, CD327 (Siglec-6), and/or CD312 (EMR2). Such modifications can be introduced via a base editing event. Such a base editing event may include, without limitation, a chemical alteration to a nucleobase. In particular embodiments, the editing event may comprise the deamination of a cytosine. In some embodiments, the editing event may comprise the deamination of an adenine. In particular embodiments, the editing event may comprise a nucleobase transition. In particular embodiments, the editing event may comprise a nucleobase transversion. In particular embodiments, the editing event may comprise converting a cytosine-guanine (C-G) base pair into a thymine-adenine (T-A) base pair within the target nucleic acid molecule. In particular embodiments, the editing event may comprise converting a thymine-adenine (T-A) base pair into a cytosine-guanine (C-G) base pair within the target nucleic acid molecule. In particular embodiments, the editing event may comprise introducing a premature STOP codon within a target nucleic acid molecule. In particular embodiments, the editing event may comprise introducing a splice site within a target nucleic acid molecule. In particular embodiments, the editing event may comprise disrupting a splice site within a target nucleic acid molecule. Accordingly, in some aspects of this disclosure provide genetically engineered cells comprising a plurality of modifications in their genome that results in a loss of expression of CD33 (Siglec-3), CLL-1, CD123, CD327 (Siglec-6), and/or CD312 (EMR2), or expression of a variant form of CD33 (Siglec-3), CLL-1, CD123, CD327 (Siglec-6), and/or CD312 (EMR2) that is not recognized by an immunotherapeutic agent targeting CD33 (Siglec-3), CLL-1, CD123, CD327 (Siglec-6), and/or CD312 (EMR2).

For example, some aspects of this disclosure provide, e.g., novel cells having a modification (e.g., a stop codon or a mutated splice site) in the endogenous CD33 (Siglec-3), CLL-1, CD123, CD327 (Siglec-6), and/or CD312 (EMR2) gene(s). In particular, provided herein are cell populations comprising a plurality of genetically engineered hematopoietic stem or progenitor cells, wherein at least a portion of the cells comprise: (i) an edited CD33 (Siglec-3) gene; (ii) an edited CLL-1 gene; (iii) an edited CD123 gene; (iv) an edited CD327 (Siglec-6) gene; (v) an edited CD312 (EMR2) gene; (vi) an edited CD33 (Siglec-3) gene and an edited CLL-1 gene; (vii) an edited CD33 (Siglec-3) gene and an edited CD123 gene; (viii) an edited CD33 (Siglec-3) gene and an edited CD327 (Siglec-6) gene; (ix) an edited CD33 (Siglec-3) gene and an edited CD312 (EMR2) gene; (x) an edited CD33 (Siglec-3) gene, an edited CLL-1 gene, and an edited CD123 gene; (xi) an edited CD33 (Siglec-3) gene, an edited CLL-1 gene, an edited CD123 gene, and an edited CD327 (Siglec-6) gene; (xii) an edited CD33 (Siglec-3) gene, an edited CLL-1 gene, an edited CD123 gene, an edited CD327 (Siglec-6) gene, and an edited CD312 (EMR2) gene; or (xiii) an edited CD33 (Siglec-3) gene, an edited CLL-1 gene, an edited CD123 gene, an edited CD327 (Siglec-6) gene, and/or an edited CD312 (EMR2) gene. In some embodiments, a cell (e.g., an HSC or HPC) having a modification of CD33 (Siglec-3), CLL-1, CD123, CD327 (Siglec-6), and/or CD312 (EMR2) is made using a nuclease and/or a gRNA described herein. In some embodiments, a cell (e.g., an HSC or HPC) having a modification of CD33 (Siglec-3), CLL-1, CD123, CD327 (Siglec-6), and/or CD312 (EMR2) and a modification of a second lineage-specific cell-surface antigen is made using a nuclease and/or a gRNA described herein. In some embodiments, the modification in the genome of the cell is a mutation in a genomic sequence encoding CD33 (Siglec-3), CLL-1, CD123, CD327 (Siglec-6), and/or CD312 (EMR2). In some embodiments, the modification is effected via genome editing, e.g., using a Cas nuclease and a gRNA targeting a CD33 (Siglec-3), CLL-1, CD123, CD327 (Siglec-6), and/or CD312 (EMR2) target site provided herein or comprising a targeting domain sequence provided herein. It is understood that the cell can be made by contacting the cell itself with the nuclease and/or a gRNA, or the cell can be the daughter cell of a cell that was contacted with the nuclease and/or a gRNA. In some embodiments, a cell described herein (e.g., an HSC) is capable of reconstituting the hematopoietic system of a subject. In some embodiments, a cell described herein (e.g., an HSC) is capable of one or more of (e.g., all of): engrafting in a human subject, producing myeloid lineage cell, and producing and lymphoid lineage cells.

While the compositions, methods, strategies, and treatment modalities provided herein may be applied to any cell or cell type, some exemplary cells and cell types that are particularly suitable for genomic modification in the CD33 (Siglec-3), CLL-1, CD123, CD327 (Siglec-6), and/or CD312 (EMR2) gene according to aspects of this disclosure are described in more detail herein. The skilled artisan will understand, however, that the provision of such examples is for the purpose of illustrating some specific embodiments, and additional suitable cells and cell types will be apparent to the skilled artisan based on the present disclosure, which is not limited in this respect.

In some embodiments, a cell described herein is a human cell having a mutation in an exon of CD33 (Siglec-3), CLL-1, CD123, CD327 (Siglec-6), and/or CD312 (EMR2).

In some embodiments, a cell described herein is a human cell having a mutation in an intron of CD33 (Siglec-3), CLL-1, CD123, CD327 (Siglec-6), and/or CD312 (EMR2).

In some embodiments, a cell described herein is a human cell having a mutation in exon 1, exon 2, exon 3, exon 4, exon 5, exon 6, and/or exon 7 of CD33.

In some embodiments, a cell described herein is a human cell having a mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, and/or exon 21 of CD312 (EMR2).

In some embodiments, a cell described herein is a human cell having a mutation in exon 2 of CLL-1 and/or CD123. In some embodiments, a cell described herein is a human cell having a mutation in exon 4 of CLL-1 and/or 5 of CD123. In some embodiments, a cell described herein is a human cell having a mutation in exon 6 of CD123.

In some embodiments, a population of cells described herein comprises hematopoietic stem cells (HSCs), hematopoietic progenitor cells (HPCs), or both (HSPCs). In some embodiments, the cells are CD34+. In some embodiments, the cell is a hematopoietic cell. In some embodiments, the cell is a hematopoietic stem cell. In some embodiments, the cell is a hematopoietic progenitor cell. In some embodiments, the cell is an immune effector cell. In some embodiments, the cell is a lymphocyte. In some embodiments, the cell is a T-lymphocyte. In some embodiments, the cell is a NK cell. In some embodiments, the cell is a stem cell. In some embodiments, the stem cell is an embryonic stem cell (ESC), an induced pluripotent stem cell (iPSC), a mesenchymal stem cell, or a tissue-specific stem cell.

In some embodiments, the cell comprises only one genetic modification. In some embodiments, the cell is only genetically modified at the CD33 (Siglec-3), CLL-1, CD123, CD327 (Siglec-6), and/or CD312 (EMR2) locus. In some embodiments, the cell is genetically modified at a second locus. In some embodiments, the cell does not comprise a transgenic protein, e.g., does not comprise a CAR.

Some aspects of this disclosure provide genetically engineered hematopoietic cells comprising a modification in their genome that results in a loss of expression of CD33 (Siglec-3), CLL-1, CD123, CD327 (Siglec-6), and/or CD312 (EMR2), or expression of a variant form of CD33 (Siglec-3), CLL-1, CD123, CD327 (Siglec-6), and/or CD312 (EMR2) that is not recognized by an immunotherapeutic agent targeting CD33 (Siglec-3), CLL-1, CD123, CD327 (Siglec-6), and/or CD312 (EMR2). In some embodiments, a modified cell described herein comprises substantially no CD33 (Siglec-3), CLL-1, CD123, CD327 (Siglec-6), and/or CD312 (EMR2) protein. In some embodiments, a modified cell described herein comprises substantially no wild-type CD33 (Siglec-3), CLL-1, CD123, CD327 (Siglec-6), and/or CD312 (EMR2) protein, but comprises mutant CD33 (Siglec-3), CLL-1, CD123, CD327 (Siglec-6), and/or CD312 (EMR2) protein. In some embodiments, the mutant CD33 (Siglec-3), CLL-1, CD123, CD327 (Siglec-6), and/or CD312 (EMR2) protein is not bound by an agent that targets CD33 (Siglec-3), CLL-1, CD123, CD327 (Siglec-6), and/or CD312 (EMR2) for therapeutic purposes.

In some embodiments, the genetically engineered cells comprising a modification in their genome results in reduced cell surface expression of CD33 (Siglec-3), CLL-1, CD123, CD327 (Siglec-6), and/or CD312 (EMR2) and/or reduced binding by an immunotherapeutic agent targeting CD33 (Siglec-3), CLL-1, CD123, CD327 (Siglec-6), and/or CD312 (EMR2), e.g., as compared to a hematopoietic cell (e.g., HSC) of the same cell type but not comprising a genomic modification.

In some embodiments, the cells are hematopoietic cells, e.g., hematopoietic stem cells, hematopoietic progenitor cell (HPC), hematopoietic stem or progenitor cell. Hematopoietic stem cells (HSCs) are cells characterized by pluripotency, self-renewal properties, and/or the ability to generate and/or reconstitute all lineages of the hematopoietic system, including both myeloid and lymphoid progenitor cells that further give rise to myeloid cells (e.g., monocytes, macrophages, neutrophils, basophils, dendritic cells, erythrocytes, platelets, etc) and lymphoid cells (e.g., T cells, B cells, NK cells), respectively. HSCs are characterized by the expression of one or more cell surface markers, e.g., CD34 (e.g., CD34+), which can be used for the identification and/or isolation of HSCs, and absence of cell surface markers associated with commitment to a cell lineage. In some embodiments, a genetically engineered cell (e.g., genetically engineered HSC) described herein does not express one or more cell-surface markers typically associated with HSC identification or isolation, expresses a reduced amount of the cell-surface markers, or expresses a variant cell-surface marker not recognized by an immunotherapeutic agent targeting the cell-surface marker, but nevertheless is capable of self-renewal and can generate and/or reconstitute all lineages of the hematopoietic system.

In some embodiments, a population of cells described herein comprises a plurality of hematopoietic stem cells; in some embodiments, a population of cells described herein comprises a plurality of hematopoietic progenitor cells; and in some embodiments, a population of cells described herein comprises a plurality of hematopoietic stem cells and a plurality of hematopoietic progenitor cells.

In some embodiments, a genetically engineered cell provided herein comprises two or more genomic modifications, e.g., one or more genomic modifications in addition to a genomic modification that results in a loss of expression of CD33 (Siglec-3), CLL-1, CD123, CD327 (Siglec-6), and/or CD312 (EMR2), or expression of a variant form of CD33 (Siglec-3), CLL-1, CD123, CD327 (Siglec-6), and/or CD312 (EMR2) that is not recognized by an immunotherapeutic agent targeting CD33 (Siglec-3), CLL-1, CD123, CD327 (Siglec-6), and/or CD312 (EMR2).

In some embodiments, a genetically engineered cell provided herein comprises a genomic modification that results in a loss of expression of CD33 (Siglec-3), CLL-1, CD123, CD327 (Siglec-6), and/or CD312 (EMR2), or expression of a variant form of CD33 (Siglec-3), CLL-1, CD123, CD327 (Siglec-6), and/or CD312 (EMR2) that is not recognized by an immunotherapeutic agent targeting CD33 (Siglec-3), CLL-1, CD123, CD327 (Siglec-6), and/or CD312 (EMR2), and further comprises an expression construct that encodes a chimeric antigen receptor, e.g., in the form of an expression construct encoding the CAR integrated in the genome of the cell. In some embodiments, the CAR comprises a binding domain, e.g., an antibody fragment, that binds CD33 (Siglec-3), CLL-1, CD123, CD327 (Siglec-6), and/or CD312 (EMR2).

Some aspects of this disclosure provide genetically engineered immune effector cells comprising a modification in their genome that results in a loss of expression of CD33 (Siglec-3), CLL-1, CD123, CD327 (Siglec-6), and/or CD312 (EMR2), or expression of a variant form of CD33 (Siglec-3), CLL-1, CD123, CD327 (Siglec-6), and/or CD312 (EMR2) that is not recognized by an immunotherapeutic agent targeting CD33 (Siglec-3), CLL-1, CD123, CD327 (Siglec-6), and/or CD312 (EMR2) In some embodiments, the immune effector cell is a lymphocyte. In some embodiments, the immune effector cell is a T-lymphocyte. In some embodiments, the T-lymphocyte is an alpha/beta T-lymphocyte. In some embodiments, the T-lymphocyte is a gamma/delta T-lymphocyte. In some embodiments, the immune effector cell is a natural killer T (NKT) cell. In some embodiments, the immune effector cell is a natural killer (NK) cell. In some embodiments, the immune effector cell does not express an endogenous transgene, e.g., a transgenic protein. In some embodiments, the immune effector cell expresses a chimeric antigen receptor (CAR). In some embodiments, the immune effector cell expresses a CAR targeting CD33 (Siglec-3), CLL-1, CD123, CD327 (Siglec-6), and/or CD312 (EMR2). In some embodiments, the immune effector cell does not express a CAR targeting CD33 (Siglec-3), CLL-1, CD123, CD327 (Siglec-6), and/or CD312 (EMR2).

In some embodiments, a genetically engineered cell provided herein expresses substantially no CD33 (Siglec-3), CLL-1, CD123, CD327 (Siglec-6), and/or CD312 (EMR2) protein, e.g., expresses no CD33 (Siglec-3), CLL-1, CD123, CD327 (Siglec-6), and/or CD312 (EMR2) protein that can be measured by a suitable method, such as an immunostaining method.

In some embodiments, a genetically engineered cell provided herein expresses substantially no wild-type CD33 (Siglec-3), CLL-1, CD123, CD327 (Siglec-6), and/or CD312 (EMR2) protein, but expresses a mutant CD33 (Siglec-3), CLL-1, CD123, CD327 (Siglec-6), and/or CD312 (EMR2) protein variant, e.g., a variant not recognized by an immunotherapeutic agent targeting CD33 (Siglec-3), CLL-1, CD123, CD327 (Siglec-6), and/or CD312 (EMR2), e.g., a CAR-T cell therapeutic, or an anti-CD33 (Siglec-3), CLL-1, CD123, CD327 (Siglec-6), and/or CD312 (EMR2) antibody, antibody fragment, or antibody-drug conjugate (ADC).

In some embodiments, the HSCs are obtained from a subject, such as a human subject. Methods of obtaining HSCs are described, e.g., in PCT/US2016/057339, which is herein incorporated by reference in its entirety. In some embodiments, the HSCs are peripheral blood HSCs. In some embodiments, the mammalian subject is a non-human primate, a rodent (e.g., mouse or rat), a bovine, a porcine, an equine, or a domestic animal. In some embodiments, the HSCs are obtained from a human subject, such as a human subject having a hematopoietic malignancy. In some embodiments, the HSCs are obtained from a healthy donor. In some embodiments, the HSCs are obtained from the subject to whom the immune cells expressing the chimeric receptors will be subsequently administered. HSCs that are administered to the same subject from which the cells were obtained are referred to as autologous cells, whereas HSCs that are obtained from a subject who is not the subject to whom the cells will be administered are referred to as allogeneic cells.

In some embodiments, a population of genetically engineered cells is a heterogeneous population of cells, e.g., heterogeneous population of genetically engineered cells containing different CD33 (Siglec-3), CLL-1, CD123, CD327 (Siglec-6), and/or CD312 (EMR2) mutations. In some embodiments, at least 40%, at least 50%, at least 60%, at least 70%, at least 75%, at least 80%, at least 85%, at least 90%, or at least 95% of copies of CD33 (Siglec-3), CLL-1, CD123, CD327 (Siglec-6), and/or CD312 (EMR2) in the population of genetically engineered cells have a mutation. In some embodiments, at least 40%, at least 50%, at least 60%, at least 70%, at least 75%, at least 80%, at least 85%, at least 90%, or at least 95% of copies of CD33 (Siglec-3), CLL-1, CD123, CD327 (Siglec-6), and/or CD312 (EMR2) in the population of genetically engineered cells have a mutation effected by a genomic editing approach described herein, e.g., by a CRISPR/Cas system using a gRNA provided herein. By way of example, a population can comprise a plurality of different CD33 (Siglec-3), CLL-1, CD123, CD327 (Siglec-6), and/or CD312 (EMR2) mutations and each mutation of the plurality contributes to the percent of copies of CD33 (Siglec-3), CLL-1, CD123, CD327 (Siglec-6), and/or CD312 (EMR2) in the population of cells that have a mutation.

In some embodiments, the expression of CD33 (Siglec-3), CLL-1, CD123, CD327 (Siglec-6), and/or CD312 (EMR2) on the genetically engineered hematopoietic cell is compared to the expression of CD33 (Siglec-3), CLL-1, CD123, CD327 (Siglec-6), and/or CD312 (EMR2) on a naturally occurring hematopoietic cell (e.g., a wild-type counterpart). In some embodiments, the genetic engineering results in a reduction in the expression level of CD33 (Siglec-3), CLL-1, CD123, CD327 (Siglec-6), and/or CD312 (EMR2) by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of CD33 (Siglec-3), CLL-1, CD123, CD327 (Siglec-6), and/or CD312 (EMR2) on a naturally occurring hematopoietic cell (e.g., a wild-type counterpart). For example, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of CD33 (Siglec-3), CLL-1, CD123, CD327 (Siglec-6), and/or CD312 (EMR2) as compared to a naturally occurring hematopoietic cell (e.g., a wild-type counterpart).

In some embodiments, the genetic engineering results in a reduction in the expression level of wild-type CD33 (Siglec-3), CLL-1, CD123, CD327 (Siglec-6), and/or CD312 (EMR2) by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of the level of wild-type CD33 (Siglec-3), CLL-1, CD123, CD327 (Siglec-6), and/or CD312 (EMR2) on a naturally occurring hematopoietic cell (e.g., a wild-type counterpart). That is, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of CD33 (Siglec-3), CLL-1, CD123, CD327 (Siglec-6), and/or CD312 (EMR2) as compared to a naturally occurring hematopoietic cell (e.g., a wild-type counterpart).

In some embodiments, the genetic engineering results in a reduction in the expression level of wild-type lineage-specific cell-surface antigen (e.g., CD33 (Siglec-3), CLL-1, CD123, CD327 (Siglec-6), and/or CD312 (EMR2)) by at least 50%, at least 60%, at least 70%, at least 80%, at least 90%, at least 91%, at least 92%, at least 93%, at least 94%, at least 95%, at least 96%, at least 97%, at least 98%, or at least 99% as compared to a suitable control (e.g., a cell or plurality of cells). In some embodiments, the suitable control comprises the level of the wild-type lineage-specific cell-surface antigen measured or expected in a plurality of non-engineered cells from the same subject. In some embodiments, the suitable control comprises the level of the wild-type lineage-specific cell-surface antigen measured or expected in a plurality of cells from a healthy subject. In some embodiments, the suitable control comprises the level of the wild-type lineage-specific cell-surface antigen measured or expected in a population of cells from a pool of healthy individuals (e.g., 10, 20, 50, or 100 individuals). In some embodiments, the suitable control comprises the level of the wild-type lineage-specific cell-surface antigen measured or expected in a subject in need of a treatment described herein, e.g., an anti-CD33 (Siglec-3), CLL-1, CD123, CD327 (Siglec-6), and/or CD312 (EMR2) therapy, e.g., wherein the subject has a cancer, wherein cells of the cancer express CD33 (Siglec-3), CLL-1, CD123, CD327 (Siglec-6), and/or CD312 (EMR2).

In some embodiments, a method of genetically engineering cells described herein comprises a step of providing a wild-type cell, e.g., a wild-type hematopoietic stem or progenitor cell. In some embodiments, the wild-type cell is an un-edited cell comprising (e.g., expressing) two functional copies of a gene encoding CD33 (Siglec-3), CLL-1, CD123, CD327 (Siglec-6), and/or CD312 (EMR2).

In some embodiments, the cell used in the method is a naturally occurring cell or a non-engineered cell.

In some embodiments, the wild-type cell expresses CD33 (Siglec-3), CLL-1, CD123, CD327 (Siglec-6), and/or CD312 (EMR2), or gives rise to a more differentiated cell that expresses CD33 (Siglec-3), CLL-1, CD123, CD327 (Siglec-6), and/or CD312 (EMR2) at a level comparable to (or within 90%-110%, 80%-120%, 70%-130%, 60-140%, or 50%-150% of) a cell line expressing CD33 (Siglec-3), CLL-1, CD123, CD327 (Siglec-6), and/or CD312 (EMR2).

In some embodiments, the wild-type cell binds an antibody that binds CD33 (Siglec-3), CLL-1, CD123, CD327 (Siglec-6), and/or CD312 (EMR2) (e.g., an anti-CD33 (Siglec-3), CLL-1, CD123, CD327 (Siglec-6), and/or CD312 (EMR2) antibody), or gives rise to a more differentiated cell that binds such an antibody at a level comparable to (or within 90%-110%, 80%-120%, 70%-130%, 60-140%, or 50%-150% of) binding of the antibody to a cell line expressing CD33 (Siglec-3), CLL-1, CD123, CD327 (Siglec-6), and/or CD312 (EMR2). Antibody binding may be measured, for example, by flow cytometry or immunohistochemistry.

### Methods of Making, Treatment, and Administration

The present disclosure provides, among other things, compositions and methods for multiplex base editing and producing a genetically engineered cell. Multiplex engineering is a strategy and method where multiple genetic targets are engineered within the same cells in the same manufacturing process. Multiplex engineering could allow removal or modification of two or more distinct genes, thus allowing for targeted therapies directed at two or more separate targets to be used in combination or in sequence, which could be particularly valuable to prevent escape mechanisms involving tumor cells down-regulating target expression.

In particular, the present disclosure provides methods for multiplex base editing, which involves converting a specific DNA base into another at a targeted genomic locus. As such, base editing does not require a cut, lowering the risk of translocation errors. The method provided herein can be used to efficiently knock out expression of multiple genomic targets, such as cell surface targets, from hematopoietic stem cells (HSCs) and/or hematopoietic stem and progenitor cells (HSPCs), for example, using a single base editing step. In particular, the present disclosure provides methods for multiplex base editing to genetically modify hematopoietic stem cells (HSCs) and/or hematopoietic stem and progenitor cells (HSPCs) to remove surface targets and then provide these cells as hematopoietic stem cell transplants (HSCTs) to patients. Once these cells engraft into bone marrow, the patient's healthy cells may protected from the negative on-target off-tumor effects of a targeted immunotherapy, for example, because they no longer express the surface target, leaving only the cancerous cells exposed. Accordingly, in some embodiments, the present disclosure provides, among other things, compositions and methods for targeted therapies to selectively destroy cancerous cells while sparing healthy cells. As a result, the engineered cells described herein may be designed to limit the on-target toxicities associated with these targeted therapies, thereby enhancing their utility, and broadening their applicability. In certain embodiments, the genetically engineered cells may be administered in combination with a targeted therapeutic, such as a chimeric antigen receptor (“CAR”)-T therapy, bispecific antibodies, and antibody-drug conjugates, designed to target cell surface proteins.

Without wishing to be bound by theory, a multiplex approach may provide advantages in at least two areas. Firstly, in the context of cancer, target expression can vary in tumor cells from the same patient, a phenomenon known as tumor heterogeneity. Applying therapies such as a multi-specific CAR-T may reduce that concern. Secondly, it is theoretically possible for tumor cells to downregulate expression of a target to avoid being killed, a phenomenon known as tumor escape. Again, pursuing multiple targets simultaneously may reduce the effectiveness of the tumor escape mechanism.

In some embodiments, an effective number of CD33 (Siglec-3), CLL-1, CD123, CD327 (Siglec-6), and/or CD312 (EMR2)-modified cells described herein is administered to a subject in combination with an anti-CD33 (Siglec-3), CLL-1, CD123, CD327 (Siglec-6), and/or CD312 (EMR2) therapy, e.g., an anti-CD33 (Siglec-3), CLL-1, CD123, CD327 (Siglec-6), and/or CD312 (EMR2) cancer therapy.

In some embodiments, an effective number of cells comprising a modified CD33 and a modified second lineage-specific cell-surface antigen are administered in combination with an anti-CD33 therapy, e.g., an anti-CD33 cancer therapy. In some embodiments, the anti-CD33 therapy comprises an antibody, a bispecific T cell engager, an ADC, or an immune cell expressing a CAR.

In some embodiments, an effective number of cells comprising a modified CLL-1 and a modified second lineage-specific cell-surface antigen are administered in combination with an anti-CLL-1 therapy, e.g., an anti-CLL-1 cancer therapy. In some embodiments, the anti-CLL-1 therapy comprises an antibody, a bispecific T cell engager, an ADC, or an immune cell expressing a CAR.

In some embodiments, an effective number of cells comprising a modified CD123 and a modified second lineage-specific cell-surface antigen are administered in combination with an anti-CD123 therapy, e.g., an anti-CD123 cancer therapy. In some embodiments, the anti-CD123 therapy comprises an antibody, a bispecific T cell engager, an ADC, or an immune cell expressing a CAR.

In some embodiments, an effective number of cells comprising a modified CD327 (Siglec-6) and a modified second lineage-specific cell-surface antigen are administered in combination with an anti-CD327 (Siglec-6) therapy, e.g., an anti-CD327 (Siglec-6) cancer therapy. In some embodiments, the anti-CD327 (Siglec-6) therapy comprises an antibody, a bispecific T cell engager, an ADC, or an immune cell expressing a CAR.

In some embodiments, an effective number of cells comprising a modified CD312 (EMR2) and a modified second lineage-specific cell-surface antigen are administered in combination with an anti-CD312 (EMR2) therapy, e.g., an anti-CD312 (EMR2) cancer therapy. In some embodiments, the anti-CD312 (EMR2) therapy comprises an antibody, a bispecific T cell engager, an ADC, or an immune cell expressing a CAR.

In some embodiments, the number of genetically engineered cells provided herein that are administered to a subject in need thereof, is within the range of 10⁶-10¹¹. However, amounts below or above this exemplary range are also within the scope of the present disclosure. For example, in some embodiments, the number of genetically engineered cells provided herein, e.g., HSCs, HPCs, or immune effector cells that are administered to a subject in need thereof is about 10⁶, about 10⁷, about 10⁸, about 10⁹, about 10¹⁰, or about 10”. In some embodiments, the number of genetically engineered cells provided herein that are administered to a subject in need thereof, is within the range of 10⁶-10⁹, within the range of 10⁶-10⁸, within the range of 10⁷-10⁹, within the range of about 10⁷-10¹⁰, within the range of 10⁸-10¹⁰, or within the range of 10⁹-10¹¹.

It is understood that when agents (e.g., CD33 (Siglec-3), CLL-1, CD123, CD327 (Siglec-6), and/or CD312 (EMR2)-modified cells and an anti-CD33 (Siglec-3), CLL-1, CD123, CD327 (Siglec-6), and/or CD312 (EMR2) therapy) are administered in combination, the agent may be administered at the same time or at different times in temporal proximity. Furthermore, the agents may be admixed or in separate volumes. For example, in some embodiments, administration in combination includes administration in the same course of treatment, e.g., in the course of treating a cancer with an anti-CD33 therapy, the subject may be administered an effective number of CD33-modified cells concurrently or sequentially, e.g., before, during, or after the treatment, with the anti-CD33 therapy. In some embodiments, administration in combination includes administration in the same course of treatment, e.g., in the course of treating a cancer with an anti-CLL-1 therapy, the subject may be administered an effective number of CLL-1-modified cells concurrently or sequentially, e.g., before, during, or after the treatment, with the anti-CLL-1 therapy. In some embodiments, administration in combination includes administration in the same course of treatment, e.g., in the course of treating a cancer with an anti-CD123 therapy, the subject may be administered an effective number of CD123-modified cells concurrently or sequentially, e.g., before, during, or after the treatment, with the anti-CD123 therapy. In some embodiments, administration in combination includes administration in the same course of treatment, e.g., in the course of treating a cancer with an anti-CD327 (Siglec-6) therapy, the subject may be administered an effective number of CD327 (Siglec-6)-modified cells concurrently or sequentially, e.g., before, during, or after the treatment, with the anti-CD327 (Siglec-6) therapy. In some embodiments, administration in combination includes administration in the same course of treatment, e.g., in the course of treating a cancer with an anti-CD312 (EMR2) therapy, the subject may be administered an effective number of CD312 (EMR2)-modified cells concurrently or sequentially, e.g., before, during, or after the treatment, with the anti-CD312 (EMR2) therapy.

In some embodiments, the agent that targets a CD33 (Siglec-3), CLL-1, CD123, CD327 (Siglec-6), and/or CD312 (EMR2) as described herein is an immune cell that expresses a chimeric receptor, which comprises an antigen-binding fragment (e.g., a single-chain antibody) capable of binding to CD33 (Siglec-3), CLL-1, CD123, CD327 (Siglec-6), and/or CD312 (EMR2). The immune cell may be, e.g., a T cell (e.g., a CD4+ or CD8+ T cell) or an NK cell.

A Chimeric Antigen Receptor (CAR) can comprise a recombinant polypeptide comprising at least an extracellular antigen binding domain, a transmembrane domain and a cytoplasmic signaling domain comprising a functional signaling domain, e.g., one derived from a stimulatory molecule. In one some embodiments, the cytoplasmic signaling domain further comprises one or more functional signaling domains derived from at least one costimulatory molecule, such as 4-1BB (i.e., CD137), CD27 and/or CD28 or fragments of those molecules. The extracellular antigen binding domain of the CAR may comprise a CD33 (Siglec-3), CLL-1, CD123, CD327 (Siglec-6), and/or CD312 (EMR2)-binding antibody fragment. The antibody fragment can comprise one or more CDRs, the variable region (or portions thereof), the constant region (or portions thereof), or combinations of any of the foregoing.

Exemplary CD33 CAR constructs are found, e.g., in PCT Publication No. WO2019/178382, incorporated herein by reference in its entirety.

Exemplary CLL-1 CAR constructs are found, e.g., in PCT Application No. PCT/CN2014/082602, and U.S. Publication No. 20160051651A1 incorporated herein by reference in its entirety.

Amino acid and nucleic acid sequences of an exemplary heavy chain variable region and light chain variable region of an anti-human CLL-1 antibody are provided below. The CDR sequences are shown in boldface in the amino acid sequences.

Amino acid sequence of anti-CLL-1 Heavy Chain Variable Region (SEQ ID NO: 3032) DIQLQESGPGLVKPSQSLSLTCSVTGYSITSAYYWNWIRQFPGNKLEWMGYISYDGRN NYNPSLKNRISITRDTSKNQFFLKLNSVTTEDTATYYCAKEGDYDVGNYYAMDYWGQ GTSVTVSS
Amino acid sequence of anti-CLL-1 Light Chain Variable Region (SEQ ID NO: 3033) ENVLTQSPAIMSASPGEKVTMTCRASSNVISSYVHWYQQRSGASPKLWIYSTSNLASGV PARFSGSGSGTSYSLTISSVEAEDAATYYCQQYSGYPLTFGAGTKLEL

Additional anti-CLL-1 sequences are found, e.g., in U.S. Pat. No. 8,536,310, which is incorporated herein by reference in its entirety.

The anti-CLL-1 antibody binding fragment for use in constructing the agent that targets CLL-1 as described herein may comprise the same heavy chain and/or light chain CDR regions as those in SEQ ID NO:3032 and SEQ ID NO:3033. Such antibodies may comprise amino acid residue variations in one or more of the framework regions. In some instances, the anti-CLL-1 antibody fragment may comprise a heavy chain variable region that shares at least 70% sequence identity (e.g., 75%, 80%, 85%, 90%, 95%, or higher) with SEQ ID NO:3032 and/or may comprise a light chain variable region that shares at least 70% sequence identity (e.g., 75%, 80%, 85%, 90%, 95%, or higher) with SEQ ID NO:3033.

Amino acid and nucleic acid sequences of an exemplary heavy chain variable region and light chain variable region of an anti-human CD123 antibody are provided below. The CDR sequences are shown in boldface in the amino acid sequences.

Amino acid sequence of anti-CD123 Heavy
Chain Variable Region
(SEQ ID NO: 2032)
MADYKDIVMTQSHKFMSTSVGDRVNITCKASQNVDSAVAWYQQKPGQSPK
ALIYSASYRYSGVPDRFTGRGSGTDFTLTISSVQAEDLAVYYCQQYYSTP
WTFGGGTKLEIKR
Amino acid sequence of anti-CD123 Light
Chain Variable Region
(SEQ ID NO: 2033)
EVKLVESGGGLVQPGGSLSLSCAASGFTFTDYYMSWVRQPPGKALEWLAL
IRSKADGYTTEYSASVKGRFTLSRDDSQSILYLQMNALRPEDSATYYCAR
DAAYYSYYSPEGAMDYWGQGTSVTVSS
Additional anti-CD123 sequences are found, e.g.,
in PCT Publication No. WO2015/140268A1,
incorporated herein by reference in its entirety.

The anti-CD123 antibody binding fragment for use in constructing the agent that targets CD123 as described herein may comprise the same heavy chain and/or light chain CDR regions as those in SEQ ID NO:2032 and SEQ ID NO:2033. Such antibodies may comprise amino acid residue variations in one or more of the framework regions. In some instances, the anti-CD123 antibody fragment may comprise a heavy chain variable region that shares at least 70% sequence identity (e.g., 75%, 80%, 85%, 90%, 95%, or higher) with SEQ ID NO:2032 and/or may comprise a light chain variable region that shares at least 70% sequence identity (e.g., 75%, 80%, 85%, 90%, 95%, or higher) with SEQ ID NO:2033.

Exemplary chimeric receptor component sequences are provided in Table 3 below.

TABLE 3
Exemplary components of a chimeric receptor

| Chimeric receptor component | Amino acid sequence |
|---|---|
| Antigen-binding fragment | Light chain- GSTSSGS |
| | GKPGSGEGSTKG (SEQ ID |
| | NO: 34)-Heavy chain |
| 4-1BB costimulatory domain | KRGRKKLLYIFKQPFMRPVQ |
| | TTQEEDGCSCRFPEEEEGGC |
| | E (SEQ ID NO: 295) |
| CD28 costimulatory domain | IEVMYPPPYLDNEKSNGTII |
| | HVKGKHLCPSPLFPGPSKPF |
| | WVLVVVGGVLACYSLLVTVA |
| | FIIFWVRSKRSRLLHSDYMN |
| | MTPRRPGPTRKHYQPYAPPR |
| | DFAAYRS (SEQ ID NO: |
| | 35) |
| ICOS costimulatory domain | LSIFDPPPFKVTLTGGYLHI |
| (boldface), ICOS trans- | YESQLCCQLKFWLPIGCAAF |
| membrane domain (italics) | VVVCILGCILICWLTKKKYS |
| and a portion of the extra- | SSVHDPNGEYMFMRAVNTAK |
| cellular domain of ICOS | KSRLTDVTL (SEQ ID |
| (underlined) | NO: 36) |
| ICOS costimulatory domain | CWLTKKKYSSSVHDPNGEYM |
| | FMRAVNTAKKSRLTDVTL |
| | (SEQ ID NO: 37) |
| CD28/ICOS chimera (the ICOS | IEVMYPPPYLDNEKSNGTII |
| portion shown in underline) | HVKGKHLCPSPLFPGPSKPF |
| including the hinge domain | WVLVVVGGVLACYSLLVTVA |
| (italics) and transmembrane | FIIFWVRSKRSRLLHSDYMF |
| domain (bold) from CD28 | MRAVNTAKKSRLTDVTL |
| | (SEQ ID NO: 38) |
| CD8α transmembrane domain | TTTPAPRPPTPAPTIASQPL |
| (italics) and a portion of | SLRPEACRPAAGGAVHTRGL |
| the extracellular domain of | DFACDIYIWAPLAGTCGVLL |
| CD8α (underlined) | LSLVITLYC (SEQ ID |
| | NO: 296) |
| CD3ζ cytoplasmic signaling | RVKFSRSADAPAYQQGQNQL |
| domain | YNELNLGRREEYDVLDKRRG |
| | RDPEMGGKPQRRKNPQEGLY |
| | NELQKDKMAEAYSEIGMKGE |
| | RRRGKGHDGLYQGLSTATKD |
| | TYDALHMQALPPR (SEQ |
| | ID NO: 39) |

In some embodiments, the CAR comprises a 4-1BB costimulatory domain (e.g., as shown in Table 3), a CD8a transmembrane domain and a portion of the extracellular domain of CD8a (e.g., as shown in Table 3), and a CD3ζ cytoplasmic signaling domain (e.g., as shown in Table 3).

A typical number of cells, e.g., immune cells or hematopoietic cells, administered to a mammal (e.g., a human) can be, for example, in the range of one million to 100 billion cells; however, amounts below or above this exemplary range are also within the scope of the present disclosure.

In some embodiments, the agent that targets CD33 (Siglec-3), CLL-1, CD123, CD327 (Siglec-6), and/or CD312 (EMR2) is an antibody-drug conjugate (ADC). The ADC may be a molecule comprising an antibody or antigen-binding fragment thereof conjugated to a toxin or drug molecule. Binding of the antibody or fragment thereof to the corresponding antigen allows for delivery of the toxin or drug molecule to a cell that presents the antigen on its cell surface (e.g., target cell), thereby resulting in death of the target cell.

Suitable antibodies and antibody fragments binding to CLL-1 will be apparent to those of ordinary skill in the art. In some embodiments, the antigen-binding fragment of the antibody-drug conjugate has the same heavy chain CDRs as the heavy chain variable region provided by SEQ ID NO: 3032 and the same light chain CDRs as the light chain variable region provided by SEQ ID NO: 3033. In some embodiments, the antigen-bind fragment of the antibody-drug conjugate has the heavy chain variable region provided by SEQ ID NO: 3032 and the same light chain variable region provided by SEQ ID NO: 3033.

Suitable antibodies and antibody fragments binding to CD123 will be apparent to those of ordinary skill in the art. In some embodiments, the antigen-bind fragment of the antibody-drug conjugate has the same heavy chain CDRs as the heavy chain variable region provided by SEQ ID NO: 2032 and the same light chain CDRs as the light chain variable region provided by SEQ ID NO: 2033. In some embodiments, the antigen-bind fragment of the antibody-drug conjugate has the heavy chain variable region provided by SEQ ID NO:2032 and the same light chain variable region provided by SEQ ID NO: 2033.

Toxins or drugs compatible for use in antibody-drug conjugates known in the art and will be evident to one of ordinary skill in the art. See, e.g., Peters et al. Biosci. Rep.(2015) 35(4): e00225; Beck et al. Nature Reviews Drug Discovery (2017) 16:315-337; Marin-Acevedo et al. J. Hematol. Oncol. (2018)11: 8; Elgundi et al. Advanced Drug Delivery Reviews (2017) 122: 2-19.

In some embodiments, the antibody-drug conjugate may further comprise a linker (e.g., a peptide linker, such as a cleavable linker) attaching the antibody and drug molecule.

Examples of antibody-drug conjugates include, without limitation, brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIV1A, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab veodtin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BIIB015, MLN2704, AMG 172, AMG 595, LOP 628, vadastuximab talirine/SGN-CD123A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-107, MEDI4276, DSTA4637S/RG7861. In one example, the antibody-drug conjugate is gemtuzumab ozogamicin.

In some embodiments, binding of the antibody-drug conjugate to the epitope of the cell-surface lineage-specific protein induces internalization of the antibody-drug conjugate, and the drug (or toxin) may be released intracellularly. In some embodiments, binding of the antibody-drug conjugate to the epitope of a cell-surface lineage-specific protein induces internalization of the toxin or drug, which allows the toxin or drug to kill the cells expressing the lineage-specific protein (target cells). In some embodiments, binding of the antibody-drug conjugate to the epitope of a cell-surface lineage-specific protein induces internalization of the toxin or drug, which may regulate the activity of the cell expressing the lineage-specific protein (target cells). The type of toxin or drug used in the antibody-drug conjugates described herein is not limited to any specific type.

CD33 (Siglec-3), CLL-1, CD123, CD327 (Siglec-6), and/or CD312 (EMR2) Associated Diseases and/or Disorders

The present disclosure provides, among other things, compositions and methods for treating a disease associated with expression of CD33 (Siglec-3), CLL-1, CD123, CD327 (Siglec-6), and/or CD312 (EMR2) or a condition associated with cells expressing CD33 (Siglec-3), CLL-1, CD123, CD327 (Siglec-6), and/or CD312 (EMR2). In some embodiments, the disease associated with expression of CD33 (Siglec-3), CLL-1, CD123, CD327 (Siglec-6), and/or CD312 (EMR2) or a condition associated with cells expressing CD33 (Siglec-3), CLL-1, CD123, CD327 (Siglec-6), and/or CD312 (EMR2) comprises, e.g., a proliferative disease such as a cancer or malignancy (e.g., a hematopoietic malignancy), or a precancerous condition such as a myelodysplasia, a myelodysplastic syndrome or a preleukemia. In some embodiments, the disease associated with expression of CD33 (Siglec-3), CLL-1, CD123, CD327 (Siglec-6), and/or CD312 (EMR2) or a condition associated with cells expressing CD33 (Siglec-3), CLL-1, CD123, CD327 (Siglec-6), and/or CD312 (EMR2) comprises, e.g., myeloproliferative neoplasms (MPN). In some embodiments, the present disclosure provides, among other things, compositions and methods for use as or in combination with a conditioning target or for the treatment of various immune disorders, e.g., based on expression profile.

In some embodiments, the hematopoietic malignancy or a hematological disorder is associated with CD33 (Siglec-3), CLL-1, CD123, CD327 (Siglec-6), and/or CD312 (EMR2) expression. A hematopoietic malignancy has been described as a malignant abnormality involving hematopoietic cells (e.g., blood cells, including progenitor and stem cells). Examples of hematopoietic malignancies include, without limitation, Hodgkin's lymphoma, non-Hodgkin's lymphoma, leukemia, or multiple myeloma. Exemplary leukemias include, without limitation, acute myeloid leukemia, acute lymphoid leukemia, chronic myelogenous leukemia, acute lymphoblastic leukemia or chronic lymphoblastic leukemia, and chronic lymphoid leukemia.

In some embodiments, cells involved in the hematopoietic malignancy are resistant to conventional or standard therapeutics used to treat the malignancy. For example, the cells (e.g., cancer cells) may be resistant to a chemotherapeutic agent and/or CAR T cells used to treat the malignancy.

In some embodiments, the leukemia is acute myeloid leukemia (AML). AML is characterized as a heterogeneous, clonal, neoplastic disease that originates from transformed cells that have progressively acquired critical genetic changes that disrupt key differentiation and growth-regulatory pathways. (Dohner et al., NEJM, (2015) 373:1136). Without wishing to be bound by theory, it is believed in some embodiments, that CD33 (Siglec-3), CLL-1, CD123, CD327 (Siglec-6), and/or CD312 (EMR2) is expressed on myeloid leukemia cells as well as on normal myeloid and monocytic precursors and is an attractive target for AML therapy.

In some cases, a subject may initially respond to a therapy (e.g., for a hematopoietic malignancy) and subsequently experience relapse. Any of the methods or populations of genetically engineered hematopoietic cells described herein may be used to reduce or prevent relapse of a hematopoietic malignancy. Alternatively or in addition, any of the methods described herein may involve administering any of the populations of genetically engineered hematopoietic cells described herein and an immunotherapeutic agent (e.g., cytotoxic agent) that targets cells associated with the hematopoietic malignancy and further administering one or more additional immunotherapeutic agents when the hematopoietic malignancy relapses. In some embodiments, the subject has or is susceptible to relapse of a hematopoietic malignancy (e.g., AML) following administration of one or more previous therapies. In some embodiments, the methods described herein reduce the subject's risk of relapse or the severity of relapse.

In some embodiments, the hematopoietic malignancy or hematological disorder associated with CD33 (Siglec-3), CLL-1, CD123, CD327 (Siglec-6), and/or CD312 (EMR2) is a precancerous condition such as a myelodysplasia, a myelodysplastic syndrome or a preleukemia. Myelodysplastic syndromes (MDS) are hematological medical conditions characterized by disorderly and ineffective hematopoiesis, or blood production. Thus, the number and quality of blood-forming cells decline irreversibly. Some patients with MDS can develop severe anemia, while others are asymptomatic. The classification scheme for MDS is known in the art, with criteria designating the ratio or frequency of particular blood cell types, e.g., myeloblasts, monocytes, and red cell precursors. MDS includes refractory anemia, refractory anemia with ring sideroblasts, refractory anemia with excess blasts, refractory anemia with excess blasts in transformation, chronic myelomonocytic leukemia (CML). In some embodiments, MDS can progress to an acute myeloid leukemia (AML).

### EXAMPLES

### Example 1: Evaluation of CD33/CLL-1 Multiplex Editing Using Base Editors

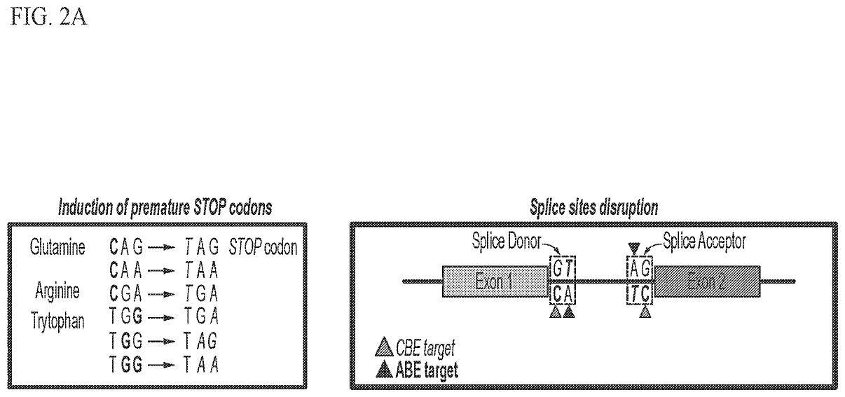

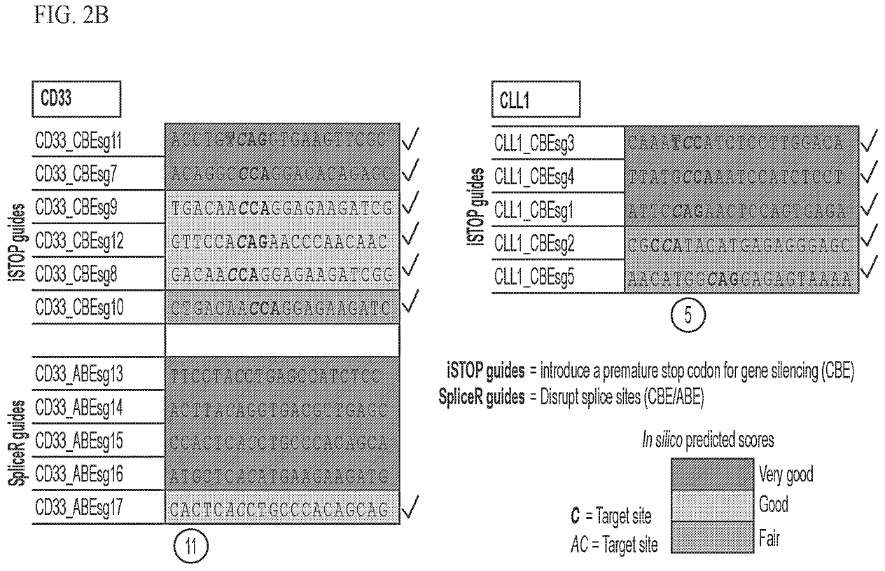

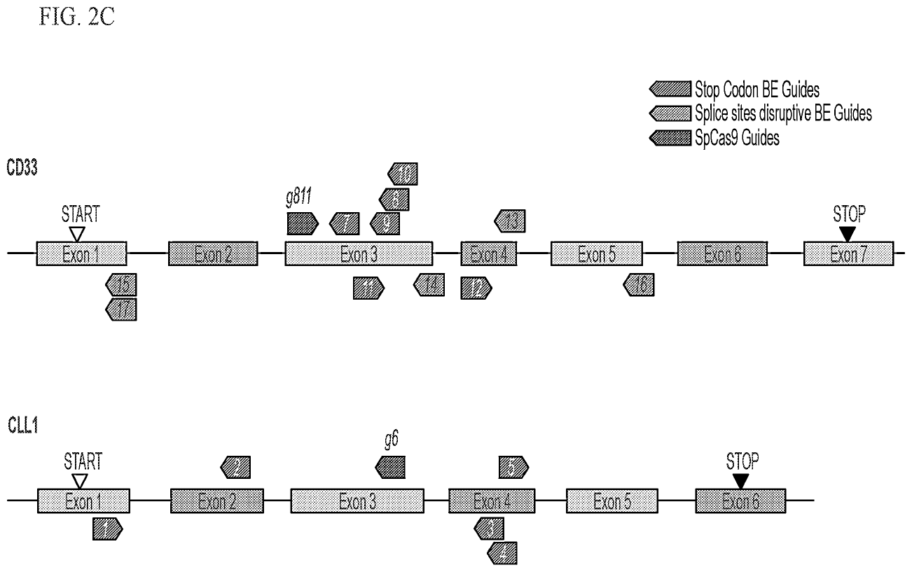

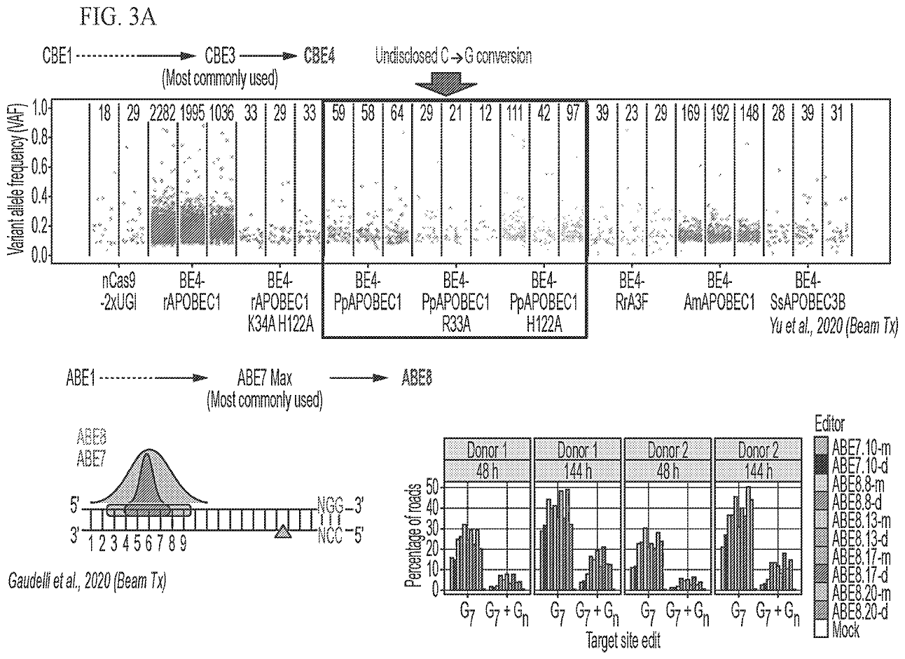

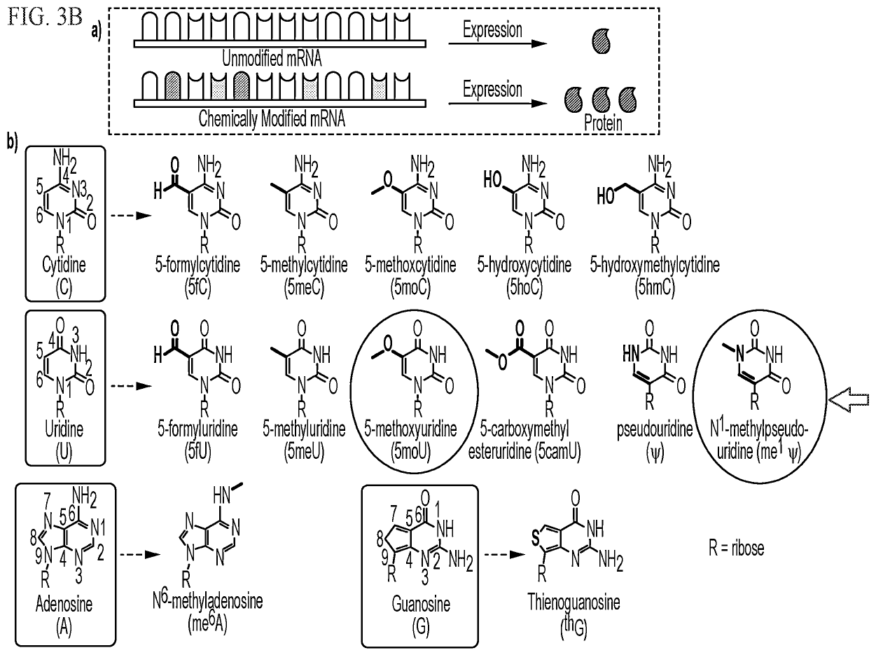

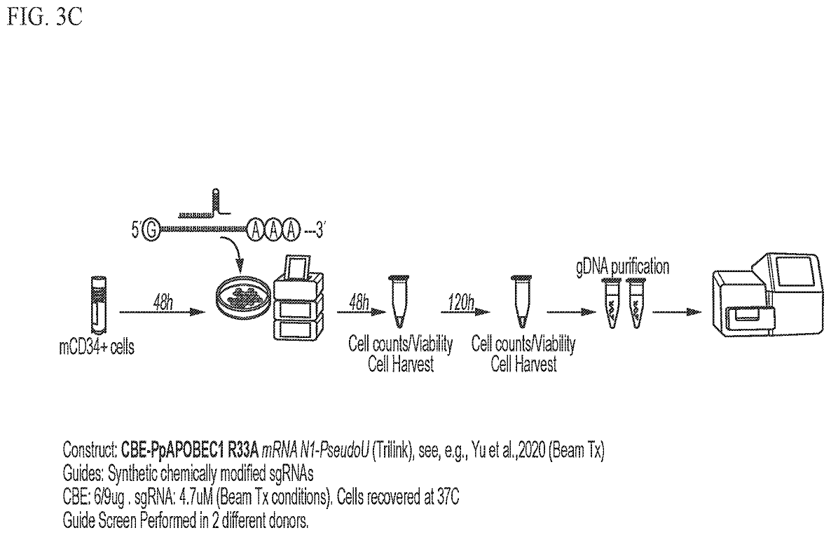

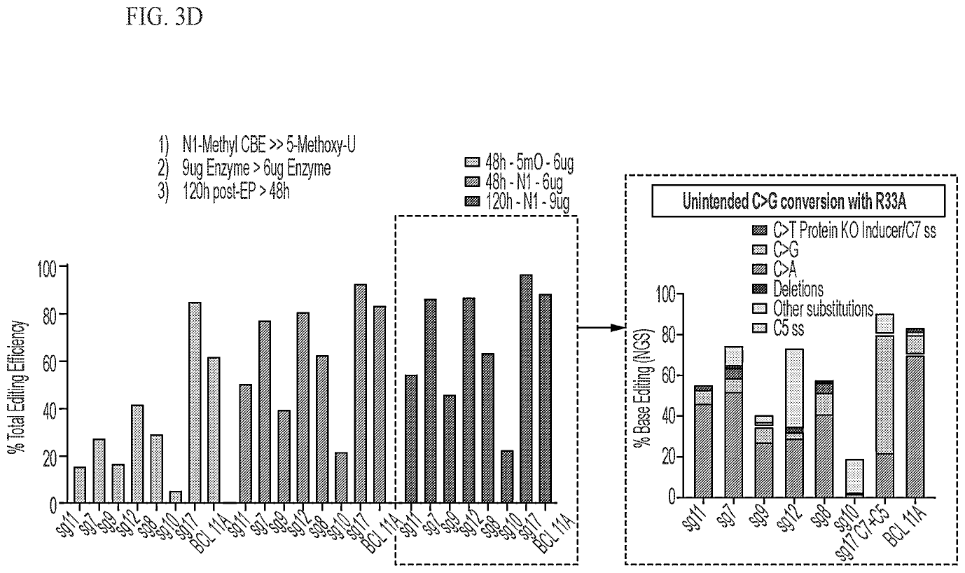

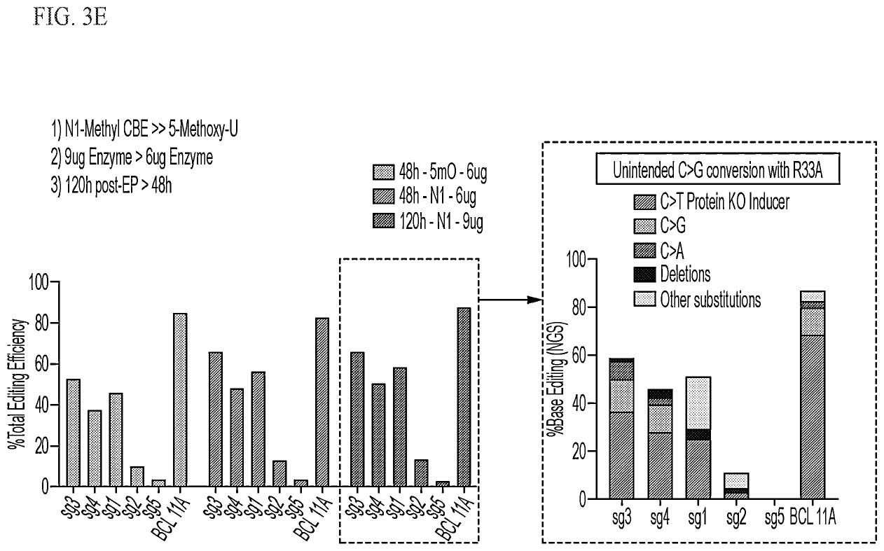

A base editing strategy was devised to evaluate base editor and guide RNA combinations for efficient single- and multiplex editing of CD33 and CLL-1. See FIGS. 1A-1C. Both cytosine base editors (CBEs) and adenine base editors (ABEs) were tested, together with guide RNAs targeting either CD33 or CLL-1. See FIGS. 2A-2C and 3A-3E. Base editor guide design and prioritization is outlined in FIGS. 2A-2C and 3A-3E, and the various guides designed for use with the various base editors (BEs) are described therein, which can be used to achieve gene knockout (KO) using base editors via the introduction of a premature STOP codon or splice site disruption. Different combinations of the guides described in FIG. 2B and Tables 1, 2, and 6-8, and the BEs disclosed in FIG. 3 were evaluated in more detail.

Guide RNA was electroporated into target cells, e.g., mobilized human CD34+ hematopoietic cells, together with mRNA encoding the respective base editor. Encoding mRNAs were chemically modified to improve expression of the encoded base editor, e.g., using 5-methoxyuridine or N1-methylpseudouridine modifications (FIG. 3B). CD34+ cells were obtained from two different donors, guide RNAs and BE-encoding mRNAs were electroporated into the cells, and cells were analyzed at different time points after electroporation, e.g., at 48 hrs and at 120 hrs. Analyses included cell viability, cell counts, and target protein expression (e.g., CD33, and CLL-1). Genomic DNA (gDNA) was obtained from edited cell populations to analyze genomic editing, e.g., via DNA sequencing.

FIG. 3D shows that high on-target CD33 base editing in HSPCs using guides 7, 8, and 17 in combination with a CBE comprising an R33A substitution. Observed editing efficiencies were higher when N1-methylpseudouridine-modified mRNA was administered to the cells as compared to the delivery of 5-methoxyuridine-modified mRNA, and the delivery of 9 micrograms of mRNA as compared to 6 micrograms of mRNA resulted in improved editing efficiencies as well. Editing efficiencies were observed to improve over time, and observed editing efficiencies were higher at 120 hrs as compared to 48 hrs after electroporation. The Sequence analysis revealed a subset of cells that comprised an unintended C-to-G conversion.

FIG. 3E shows that high on-target CLL-1 base editing in HSPCs using guides 3, 4, and 1 in combination with a CBE comprising an R33A substitution. Observed editing efficiencies were higher when N1-methylpseudouridine-modified mRNA was administered to the cells as compared to the delivery of 5-methoxyuridine-modified mRNA, and the delivery of 9 micrograms of mRNA as compared to 6 micrograms of mRNA resulted in improved editing efficiencies as well. Editing efficiencies were observed to improve over time, and observed editing efficiencies were higher at 120 hrs as compared to 48 hrs after electroporation. The Sequence analysis revealed a subset of cells that comprised an unintended C-to-G conversion.

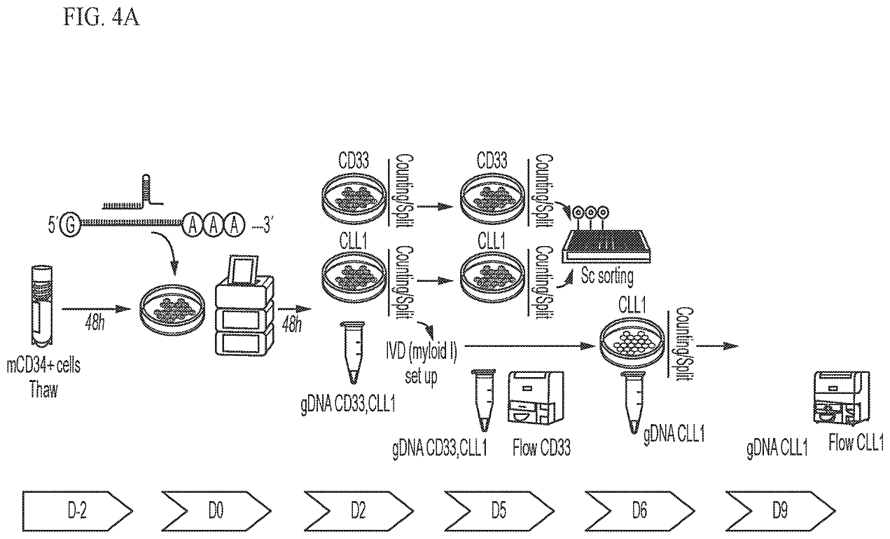

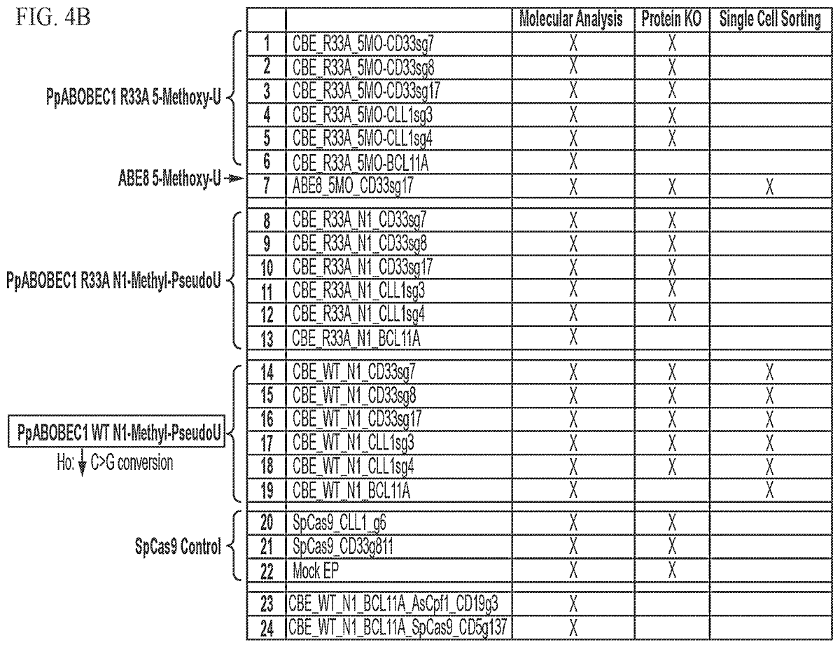

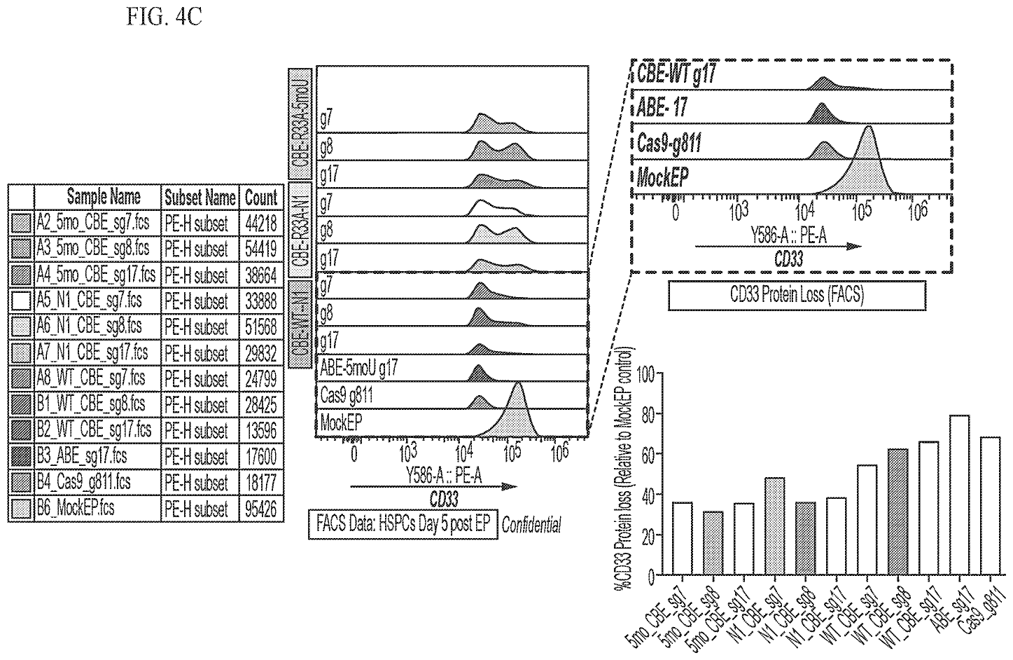

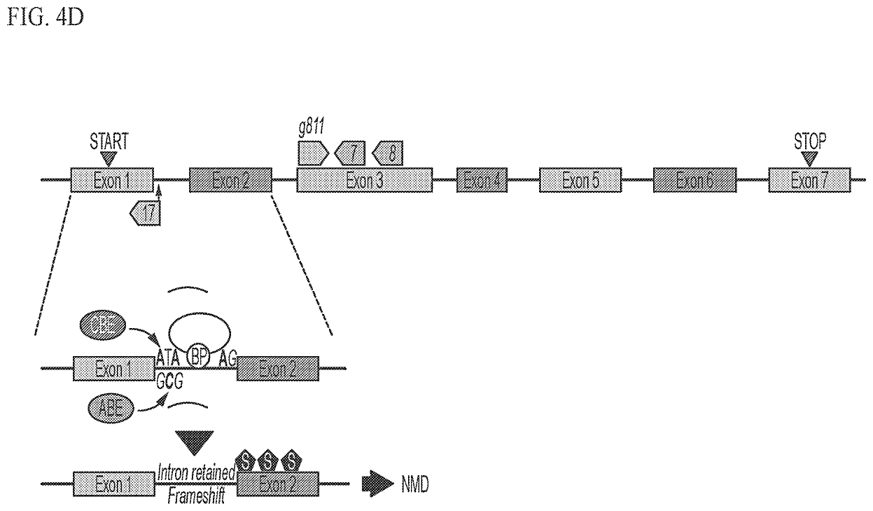

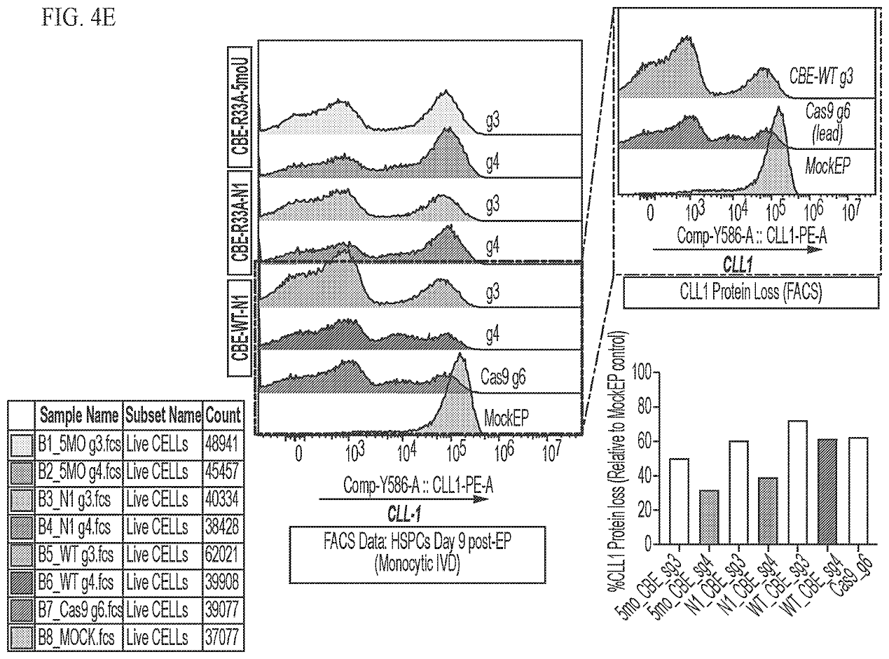

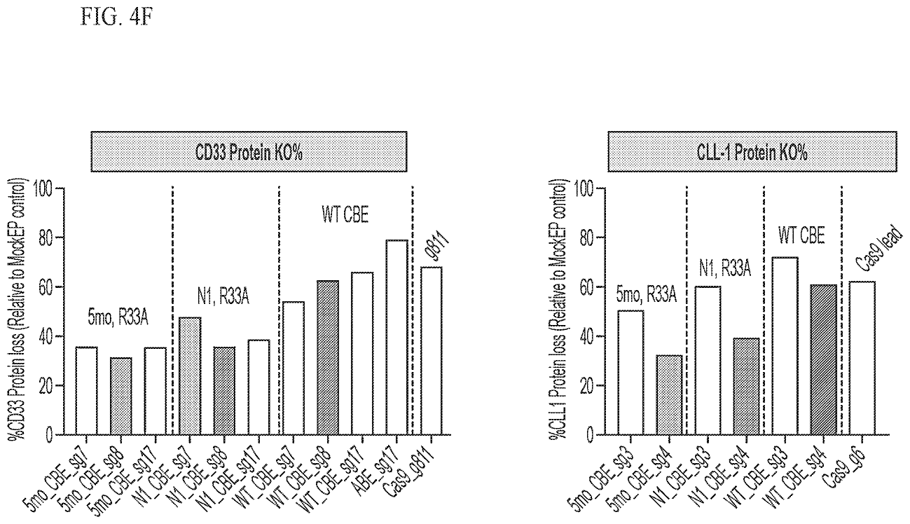

Different CBEs, e.g., “WT” and “R33A” variants, were evaluated for improved base editing efficiencies and lack of unintended base conversions. In addition, ABE editing strategies were also evaluated. FIG. 4 discloses the various combinations that were evaluated. FIG. 4A illustrates the experimental design. FIG. 4B shows the specific BE and guide combinations tested. FIG. 4C shows that cytosine/adenine base editing of CD33E1-Splice Site using different guide RNAs efficiently disrupts CD33 expression. Inset shows results for guide RNA 17. EP: electroporation. Without wishing to be bound by theory, it is contemplated that some of the edits disclosed herein result in efficient CD33 knockout via a nonsense-mediated decay mechanism, e.g., when the CD33E1 splice donor site is disrupted by CBE/ABE in combination with guide sg17. See FIG. 4D. FIG. 4E shows that cytosine base editing of CLL-1 using various guide RNAs efficiently disrupts protein expression. Inset shows results for guide RNA 3.

Base editing efficiencies, measured as the percentage of cells displaying target protein (CD33 or CLL-1) knockout, of different CBEs in combination with different guides are summarized in FIG. 4F. These data demonstrate that exemplary CD33 BE and guide combinations can achieve CD33 protein loss in HSPCs or more than 60%, and that exemplary CLL-1 BE and guide combinations can achieve more than 60% CLL-1 protein loss in HPSCs.

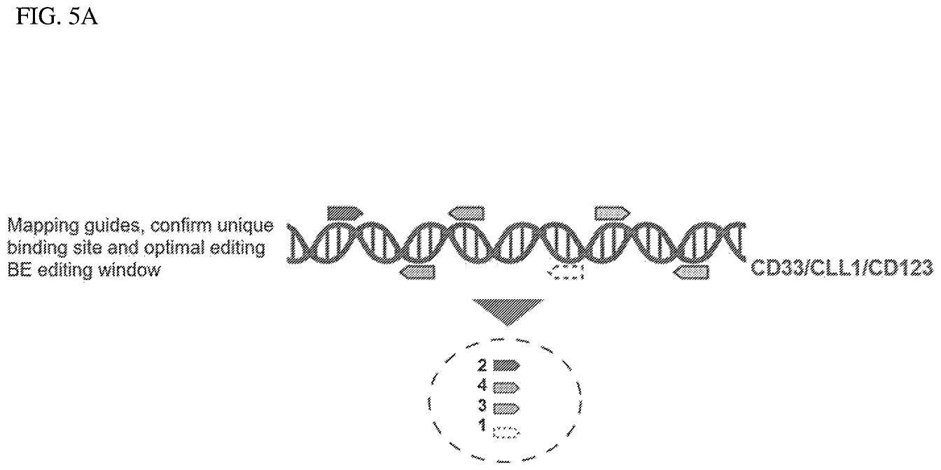

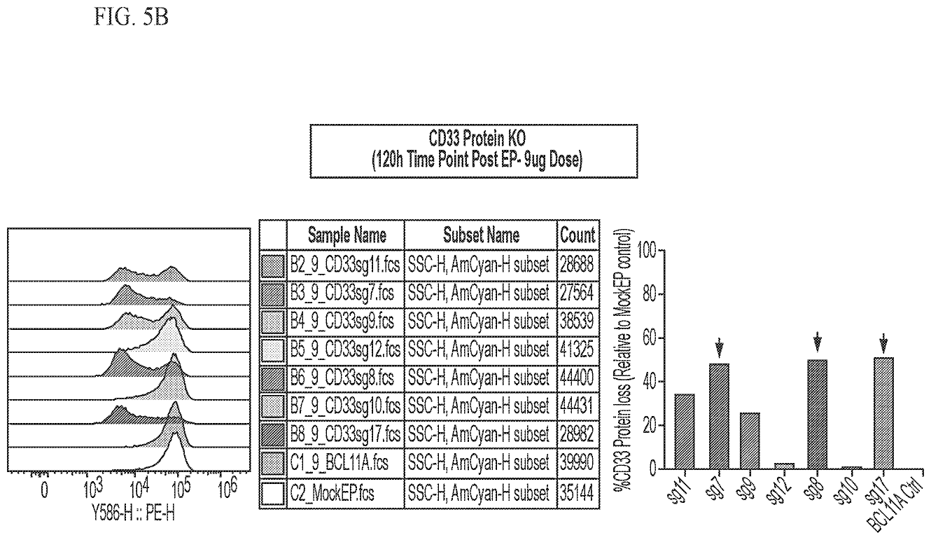

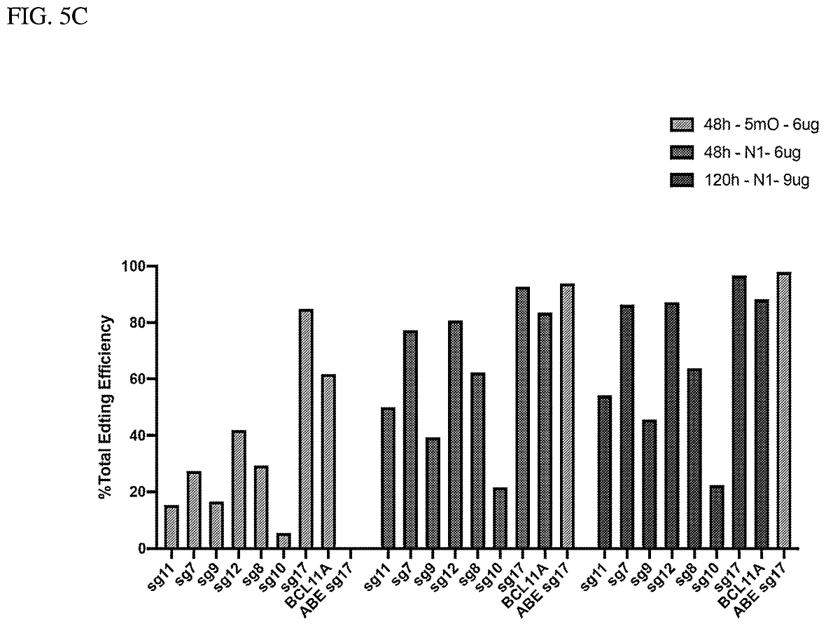

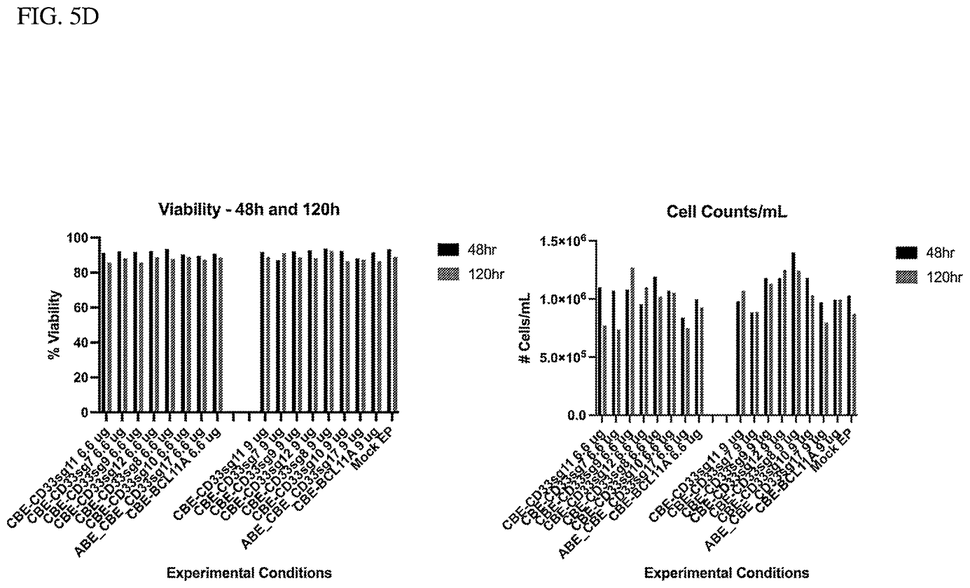

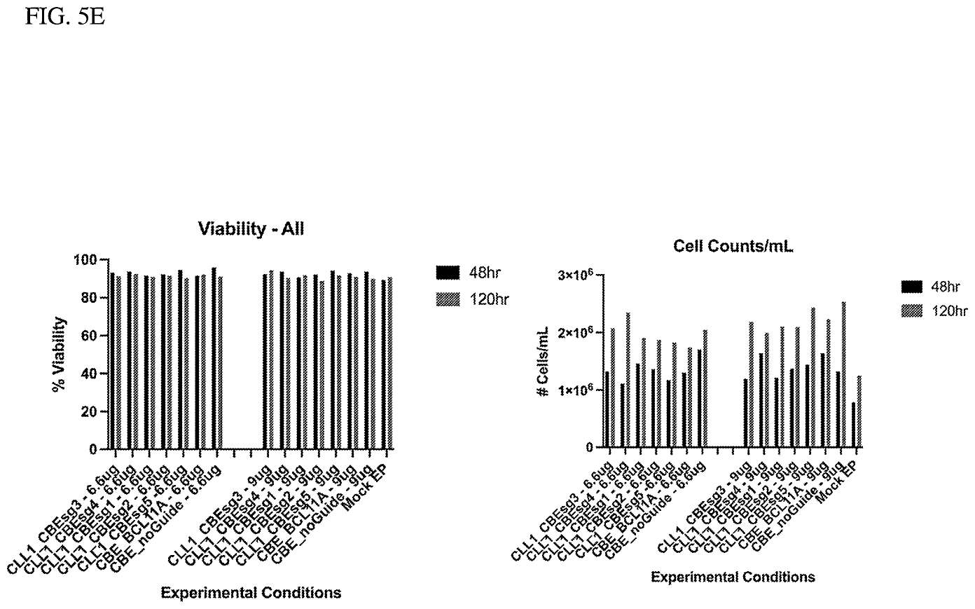

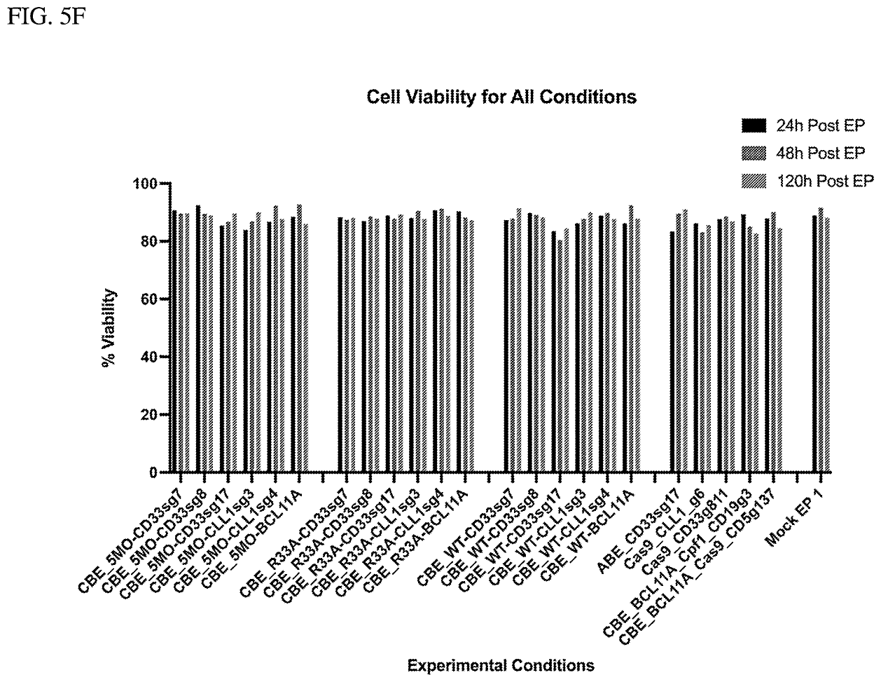

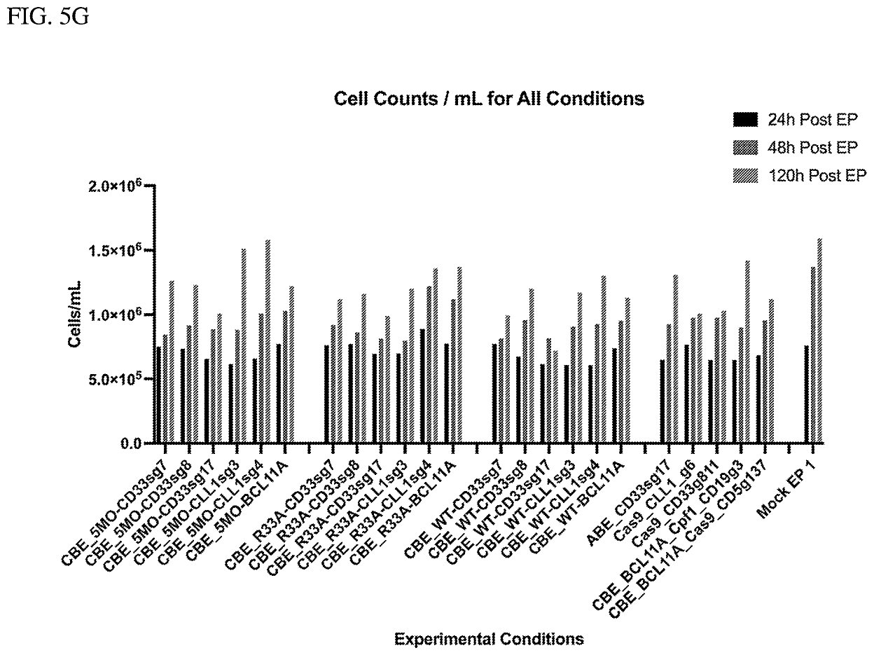

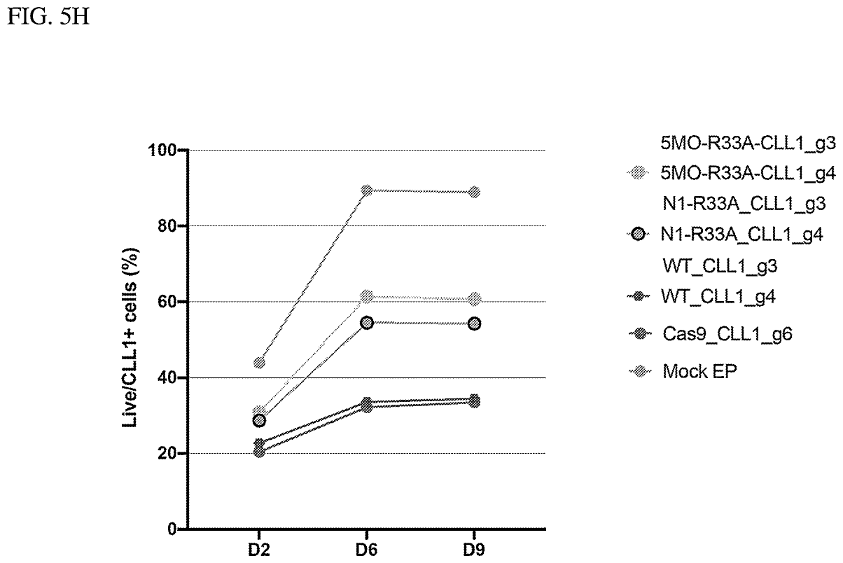

Further characterizations of gene editing efficiencies of various BE and guide RNA combinations were performed. See FIG. 5 for a summary of the results.

### Example 2: Multiplex Base Editing of CD33 and CLL-1

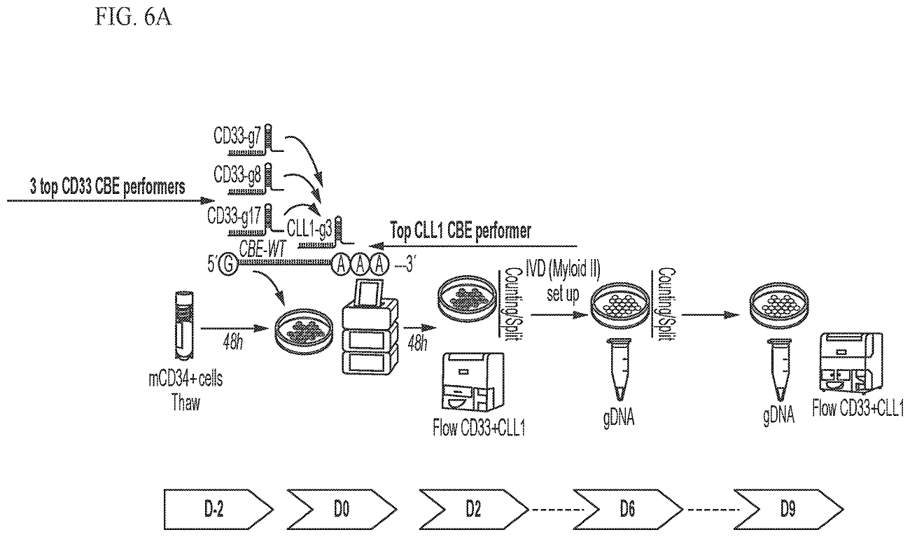

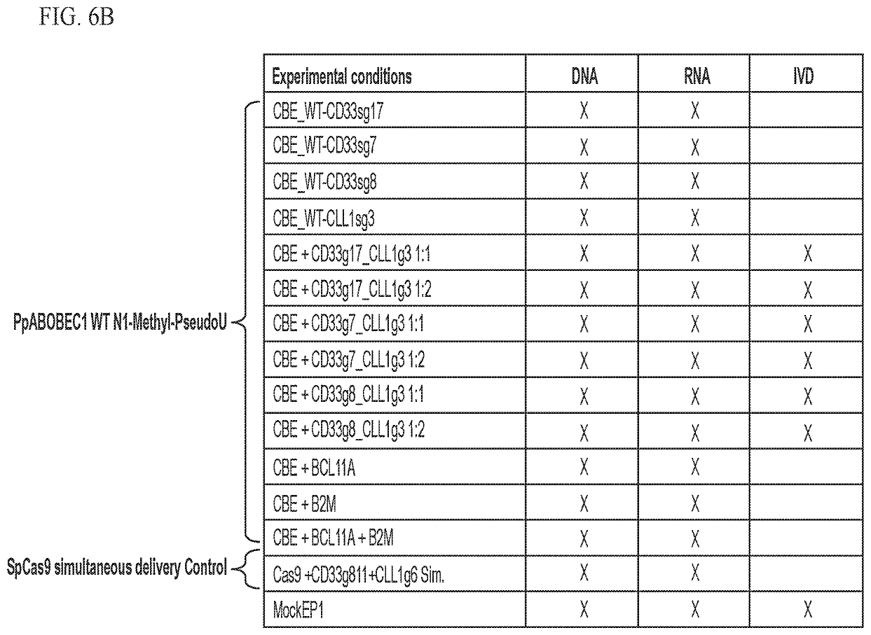

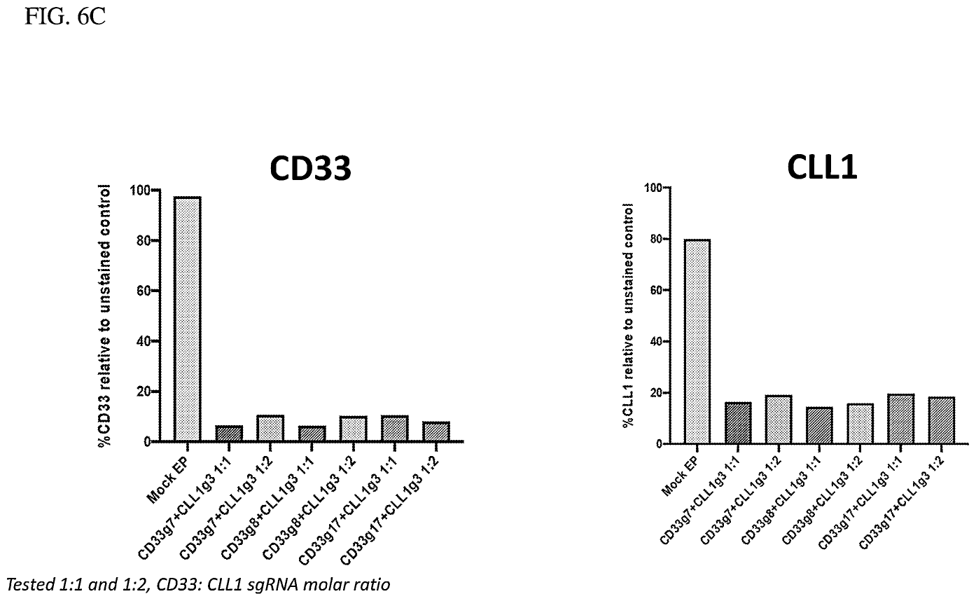

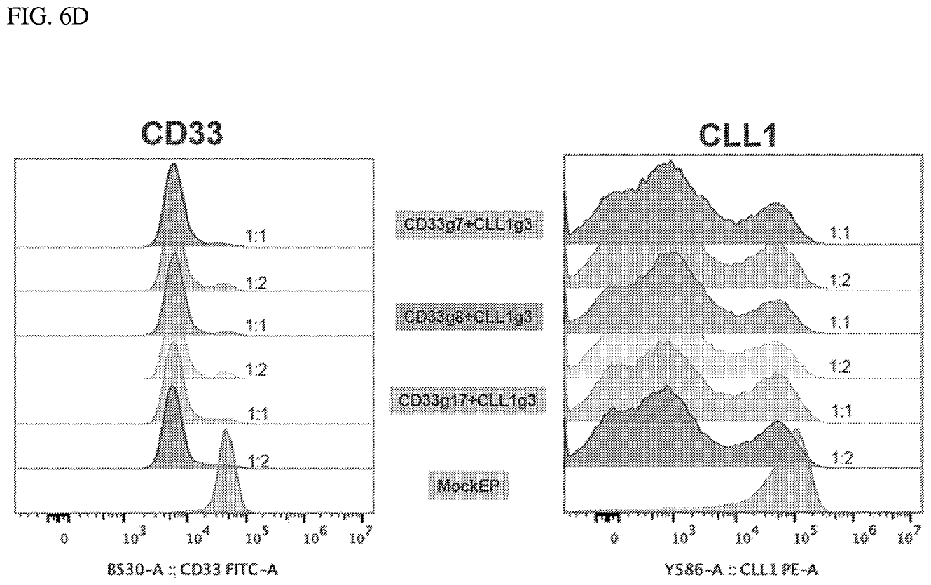

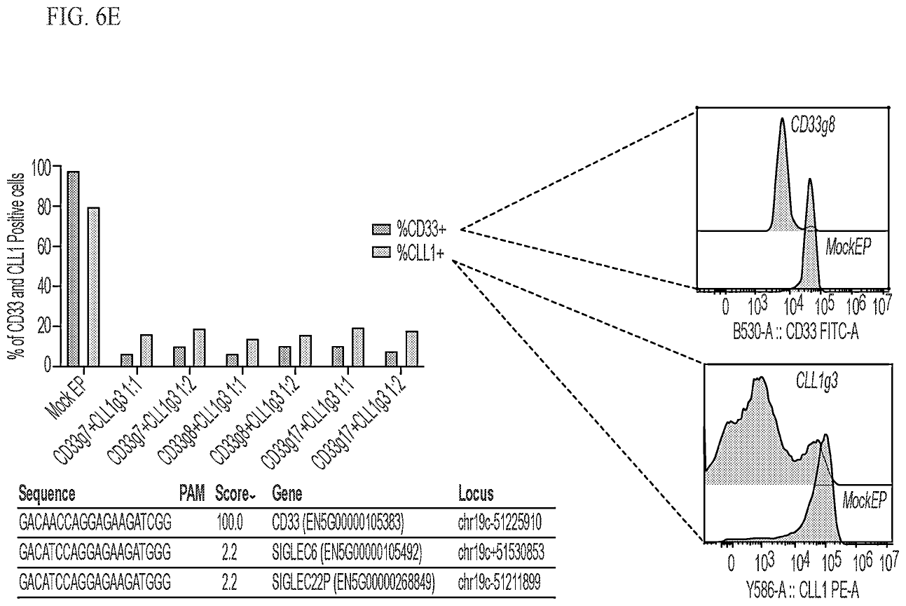

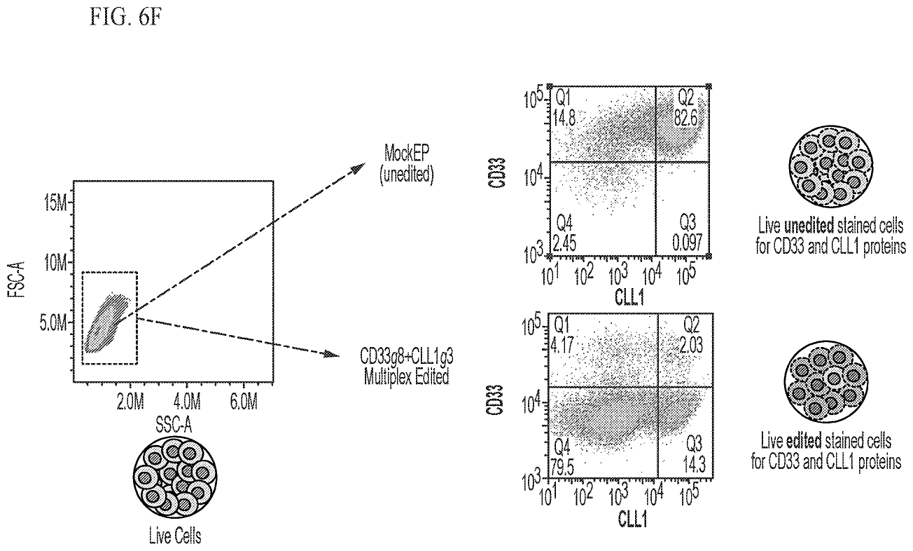

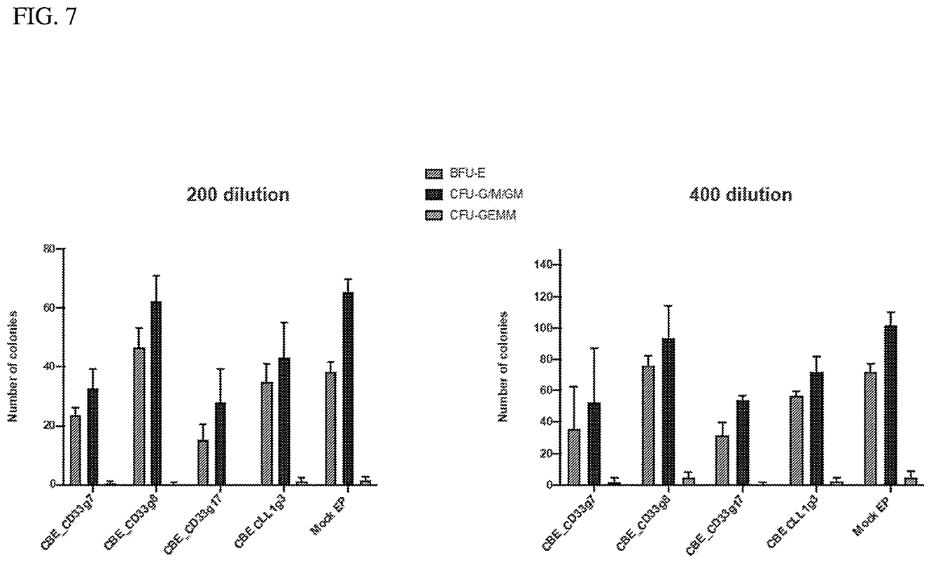

Multiplex editing of CD33 and CLL-1 was performed using different CD33 guide RNAs (sg7, sg8, or sg17), in combination with CLL-1 guide RNA sg3. FIG. 6A illustrates the experimental design. FIG. 6B shows the arms of this study, which included a dose titration multiplex base editing using the three top CBE CD33 guides in combination with the top CLL-1 guide. FIG. 6C demonstrates efficient knockout of both CD33 and CLL-1 in mobilized human CD34+ HPSCs. CD33 expression was present in less than 10% of cells in the edited HPSC cell populations, and CLL-1 expression was present in less than 20% of cells in the edited HPSC cell populations. FIG. 6D shows FACS data of the edited cell populations using various guide combinations. FIG. 6E shows CD33 and CLL-1 protein KO data in HPSCs for different ratios of guides used in the experiments. FACS data for CD33g8 and CLL-1g3 are shown on the right. Exemplary off-target analysis is shown on the bottom left for CD33sg8, demonstrating a desirable off-target profile. FACS analysis demonstrated that ˜80% of multiplex edited cells lack CD33 and CLL-1 surface protein expression (FIG. 6F). Multiplex-edited cells (CD33KO/CLL-1KO) were subjected to a colony-forming assay and data are shown in FIG. 7, demonstrating that the base edited cells were not impacted in their colony-forming and differentiation potential.

| guide RNAs | Sequence |
|---|---|
| CD33_CBEsg7 | 5′acaGGCCCAGGACACAGAGCGUUUUAGAGCUAG |
| | AAAUAGCAAGUUAAAAUAAGGCUAGUCCGUUAUCA |
| | ACUUGAAAAAGUGGCACCGAGUCGGUGCuuuU3′ |
| CD33_ABEsg17 | 5′cacUCACCUGCCCACAGCAGGUUUUAGAGCUAG |
| | AAAUAGCAAGUUAAAAUAAGGCUAGUCCGUUAUCA |
| | ACUUGAAAAAGUGGCACCGAGUCGGUGCuuuU3′ |
| CD33_CBEsg8 | 5′gacAACCAGGAGAAGAUCGGGUUUUAGAGCUAG |
| | AAAUAGCAAGUUAAAAUAAGGCUAGUCCGUUAUCA |
| | ACUUGAAAAAGUGGCACCGAGUCGGUGCuuU3′ |
| CLL-1_CBEsg3 | 5′caaAUCCAUCUCCUUGGACAGUUUUAGAGCUAG |
| | AAAUAGCAAGUUAAAAUAAGGCUAGUCCGUUAUCA |
| | ACUUGAAAAAGUGGCACCGAGUCGGUGCuuuU3′ |

### Example 3: Comboplexing—Simultaneous Delivery Cytosine Base Editor and AsCpf1

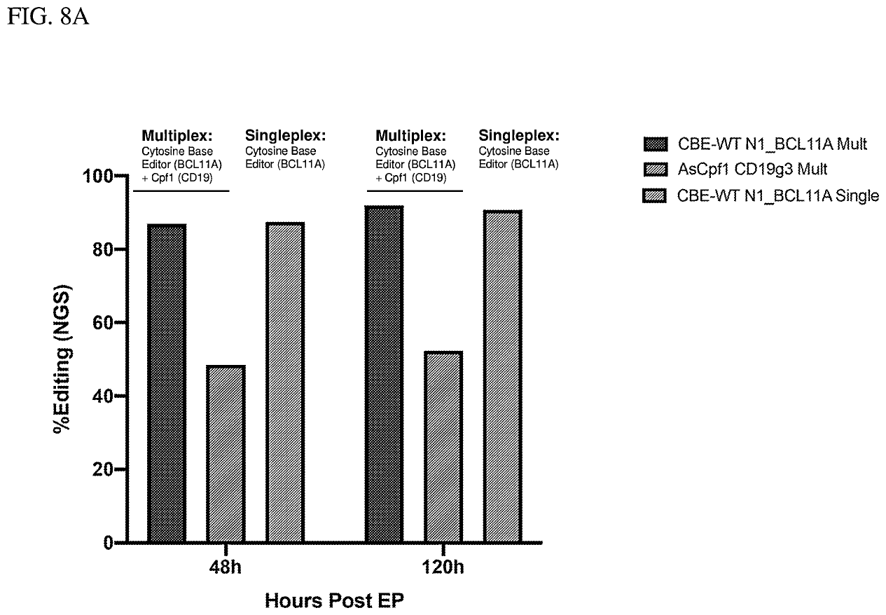

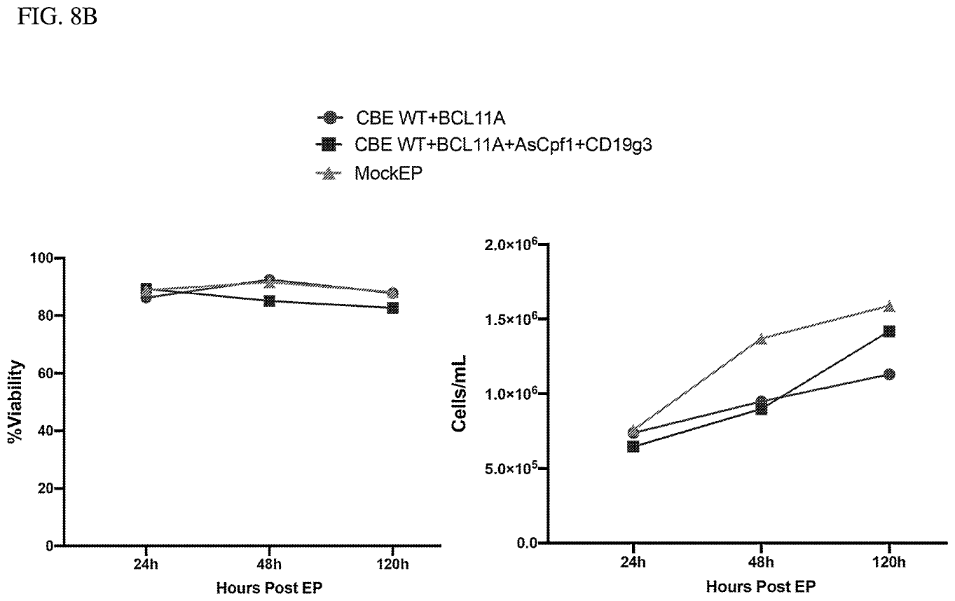

As shown in FIG. 8A, comboplexing uses simultaneous delivery of Cytosine Base Editor (CBE) and Cpf1 nuclease to allow for single delivery and no translocation risk as the base editor does not make double strand break. These data demonstrate >50% editing at both loci when the Base Editor and Cpf1 are delivered together.

As shown in FIG. 8B, viability and cell growth is not impacted when delivering simultaneously CBE and AsCpf1 in CD34 cells

### Example 4: Simultaneous Multiplex Base Editing Engineering Protocol in HSCs Using Exemplary CD33 and CLL-1 Cytosine Base Editor (CBE) Guides

Multiplex editing of CD33 and CLL-1 was performed using different CD33 guide RNAs (sg7, sg8, or sg17), in combination with CLL-1 guide RNA sg3. FIG. 9A illustrates the experimental design. After in silico guide design, at day 1, mCD34+ cells were thawed for culture. After 48-hours, the CD33 and CLL-1 guides were introduced into the mCD34+ cells for screening. After 24-hours, cell counts and cell viability were assessed. After 48-hours, cell counts and cell viability was again assessed, and the cells were harvested. gDNA was purified and the editing readout obtained via NGS. Additionally, a myeloid in vitro differentiation was set up, and after 6 days the cells were assessed by Flow Cytometry for protein knockout (KO) readout. FIG. 9B shows day 6 post myeloid in vitro differentiation data. The base editor (BE) combination of CD33g8 and CLL-1g3 showed 80% double surface protein KO. FIG. 9C shows that balanced translocations were not detected in the multiplex base edited samples as determined by a RhampSeq assay. These data demonstrate the feasibility and success achievable with simultaneous multiplex base editing. In particular, this experiment achieved approximately 80% CD33/CLL-1 double KO cells and 0% translocations, i.e., the RhampSeq assay could not detect any translocations. Notably, there was no impact on cell viability or cell expansion.

### Example 5: Multiplex Base Editing in Human Hematopoietic Stem and Progenitor Cells (HSPCs) Enables Efficient Removal of Multiple Surface Antigens in Acute Myeloid Leukemia (AML) Immunotherapy

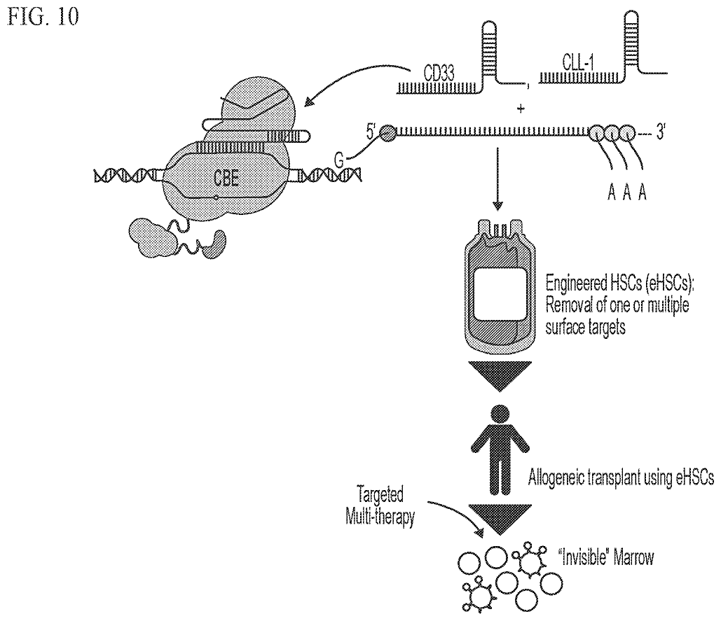

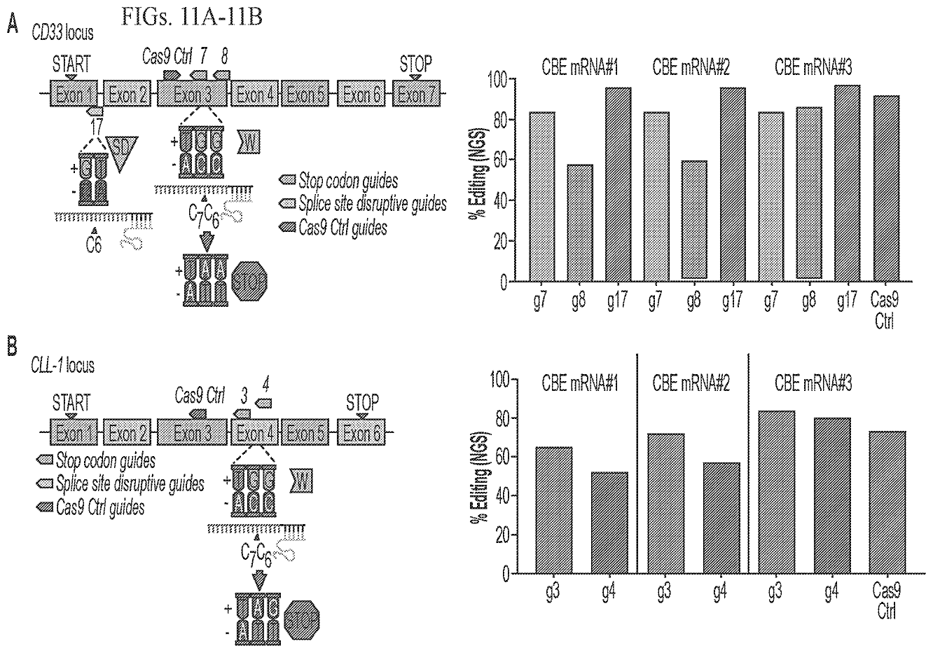

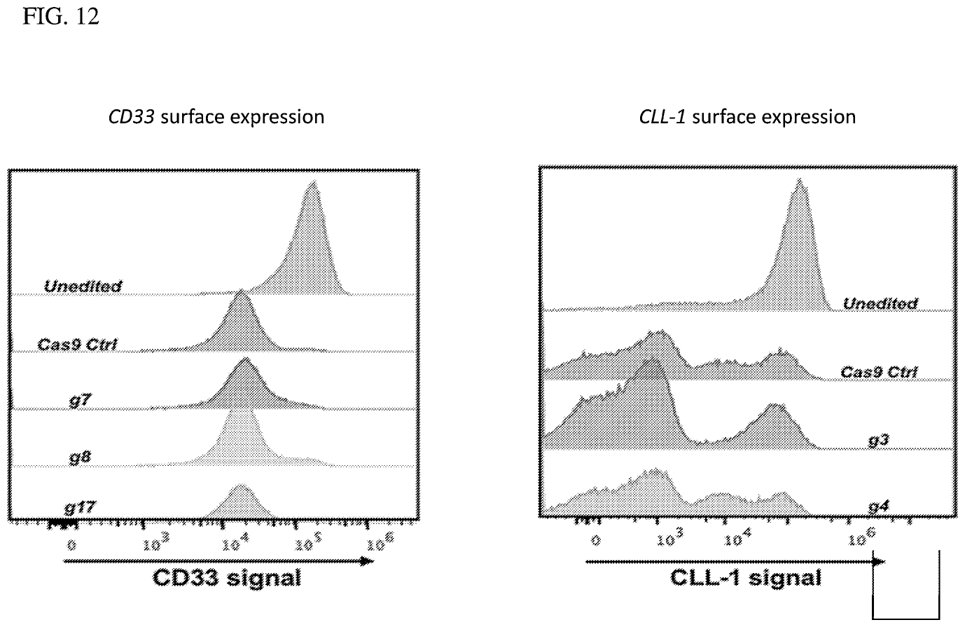

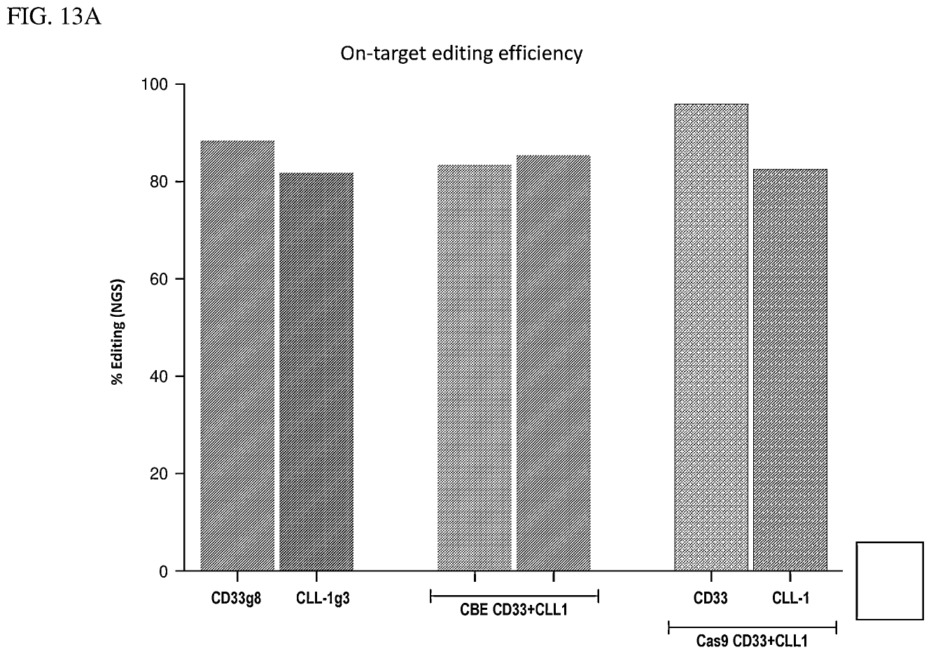

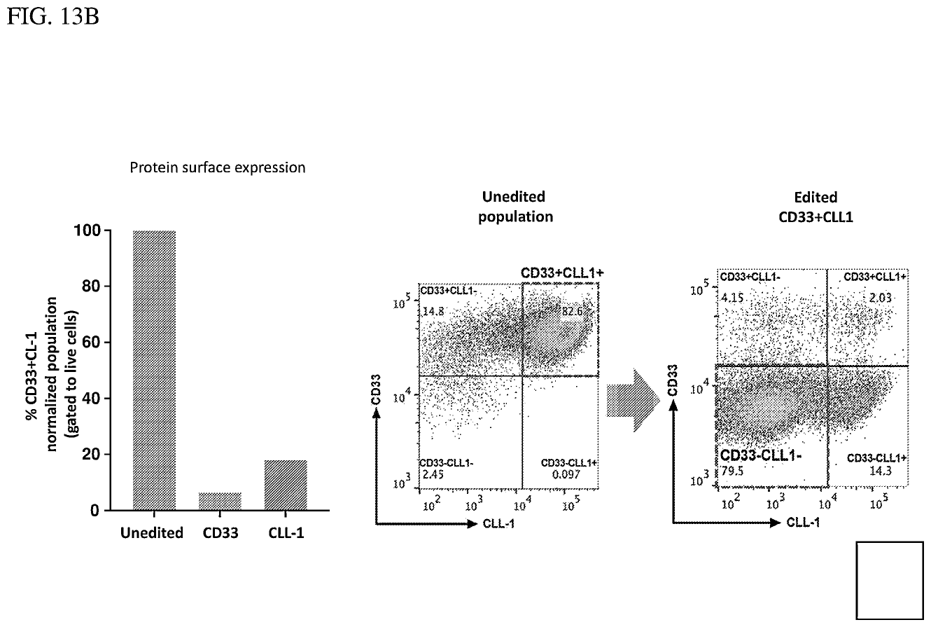

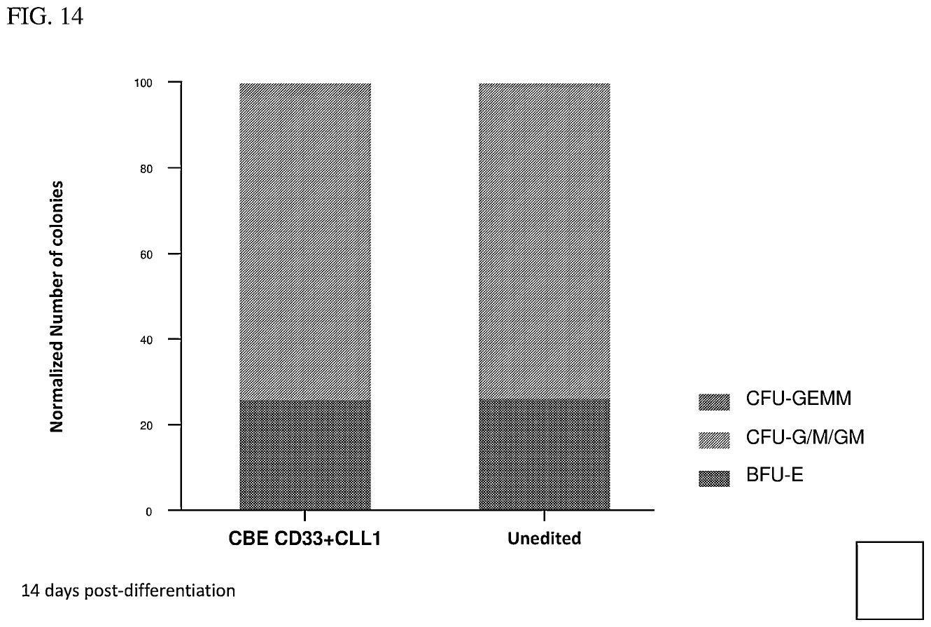

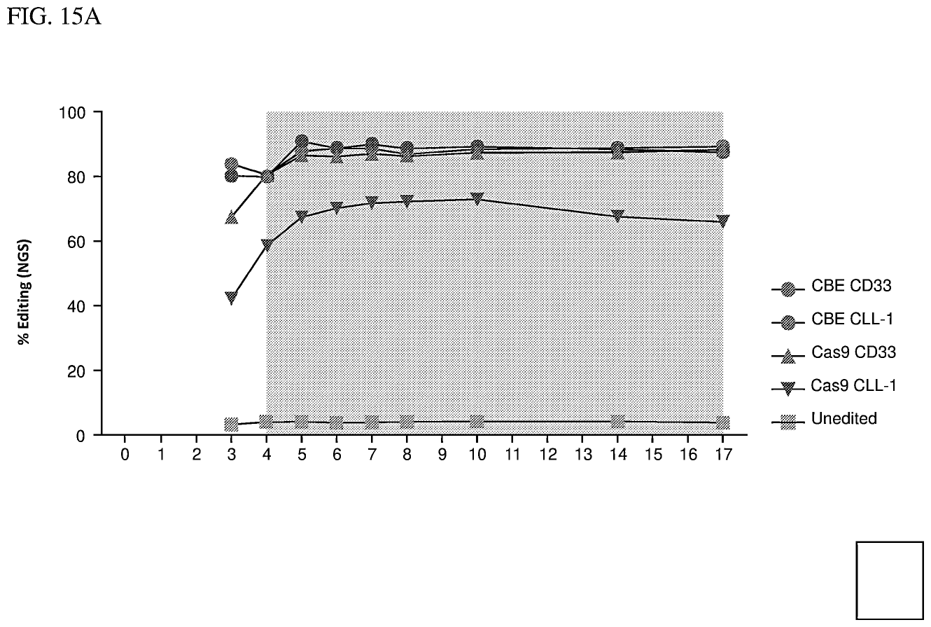

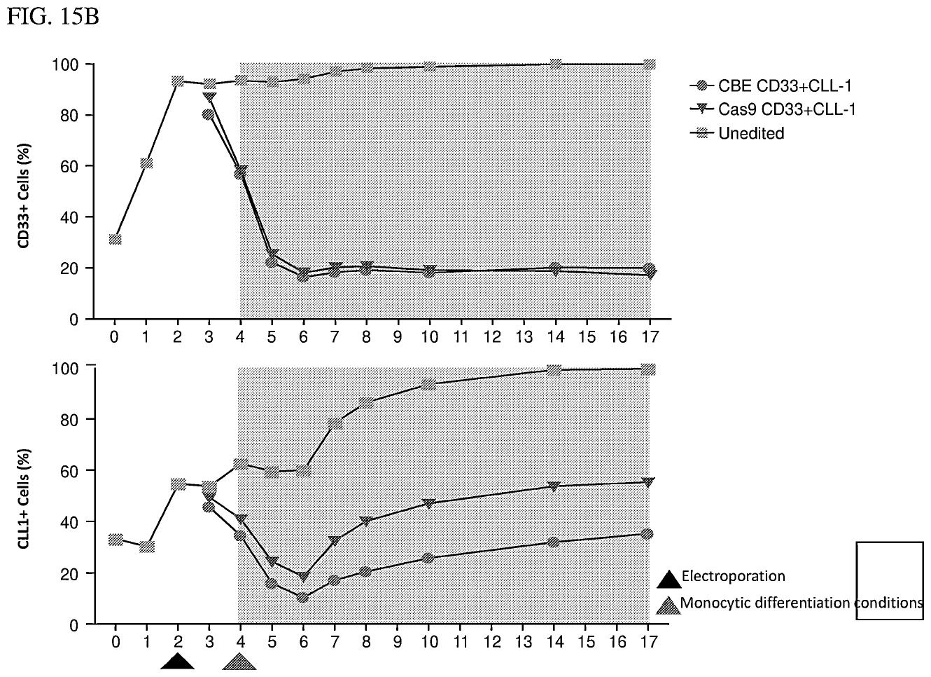

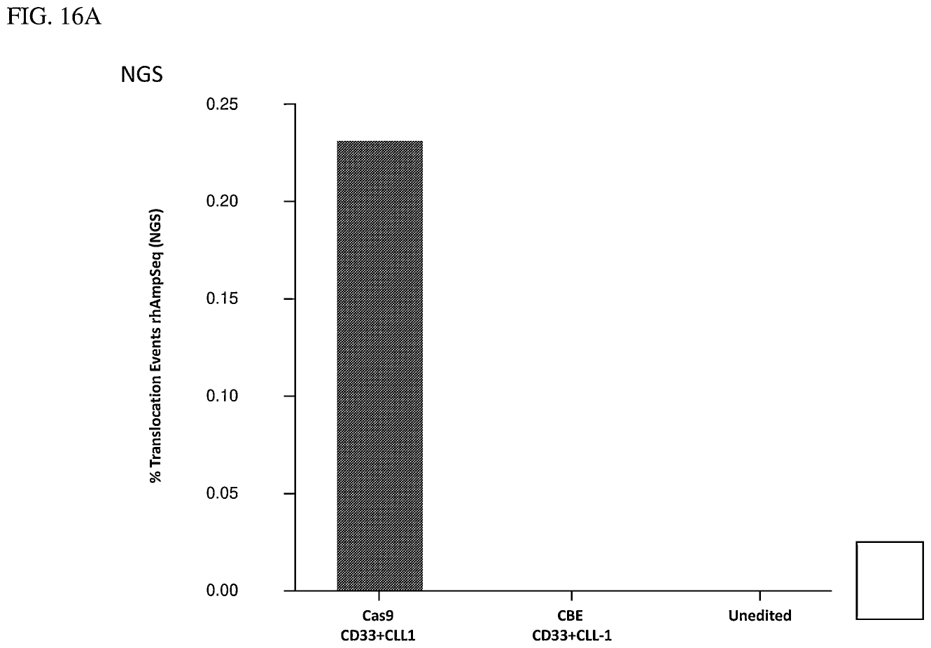

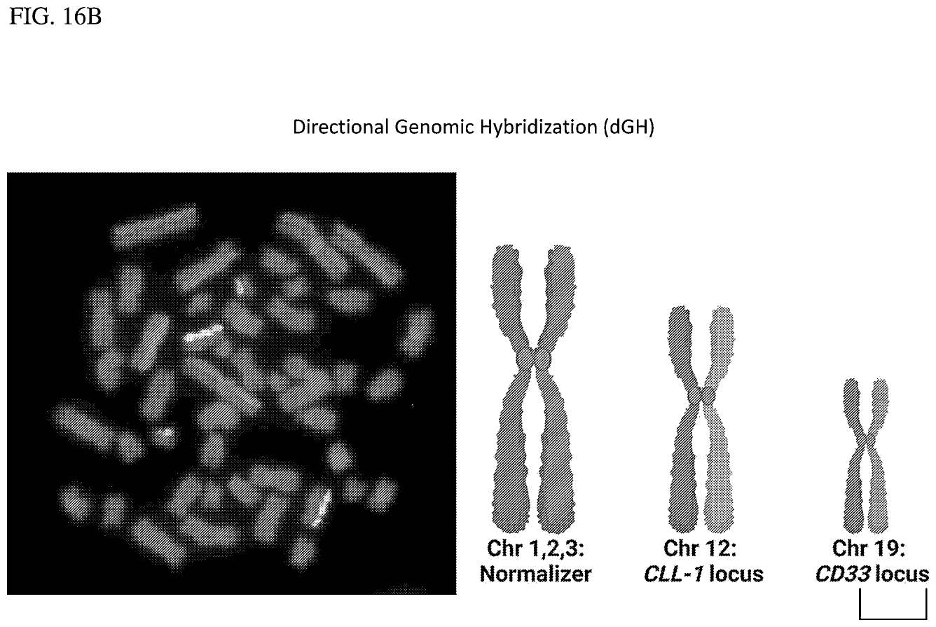

Multiplex base editing of CD34+ hematopoietic stem and progenitor cells (HSPCs) from healthy donors was performed using different CD33 and CLL-1 guide RNAs. FIG. 10 illustrates the experimental design. A CBE4 base editing guide screen of CD33 and CLL-1 was performed, and generated high efficient editing in CD34+ HSPCs. FIG. 11A shows on-target base editing efficiency of three CBE gene knockout (KO) inducing single guides (sg7, sg8, or sg17) using three different CBE4 mRNA encoding constructs, compared to Cas9-induced indel frequency on the CD33 locus. FIG. 11B shows on-target base editing efficiency of two CBE gene knockout (KO) inducing single guides (sg3, sg4) using three different CBE4 mRNA encoding constructs, compared to Cas9-induced indel frequency on the CLL-1 locus. FIG. 12 shows that the efficient base editing of CD33 and CLL-1 abrogates CD33 and CLL-1 protein surface expression. FIGS. 13A-13B illustrates that multiplex base editing of CD33 and CLL-1 loci shows efficient on-target editing and dual CD33 and CLL-1 protein surface expression knock-out. The right panel of FIG. 13B showed 80% true double KO population for edited CD33+CLL-1 (i.e., CD33-CLL-1-cells). FIG. 14 shows that the multilineage potential of double edited CD34+ HSPCs was maintained after multiplex base editing. FIGS. 15A-15B illustrates that myeloid in vitro differentiation showed editing persistence in monocytes and protein KO expression of CD33 and CLL-1 multiplexed edited cells. FIGS. 16A-16B show that translocations were not detected in CD33+CLL-1 multiplex base edited samples.

These data demonstrate the feasibility and success achievable with simultaneous multiplex base editing. In particular, these data show that simultaneous delivery of base editing guides can preserve health, expansion, and stemness of HSPCs which could facilitate the process and manufacturing of cells for therapeutic applications, such as for the treatment of AML. Additionally, this multiplex base editing experiment achieved a high base editing efficiency, robust surface protein KO, and no detection of balanced translocation of the multiplex base edited cells. Accordingly, these data demonstrate that multiplex base editing in CD34+ HSPCs of one, two, or multiple surface targets offers a valuable, safe, and efficacious alternative to engineer the next generation of transplants to treat AML patients.

### Example 6: Evaluation of CD33/CD123 Multiplex Editing Using Adenine Base Editors ABEs)

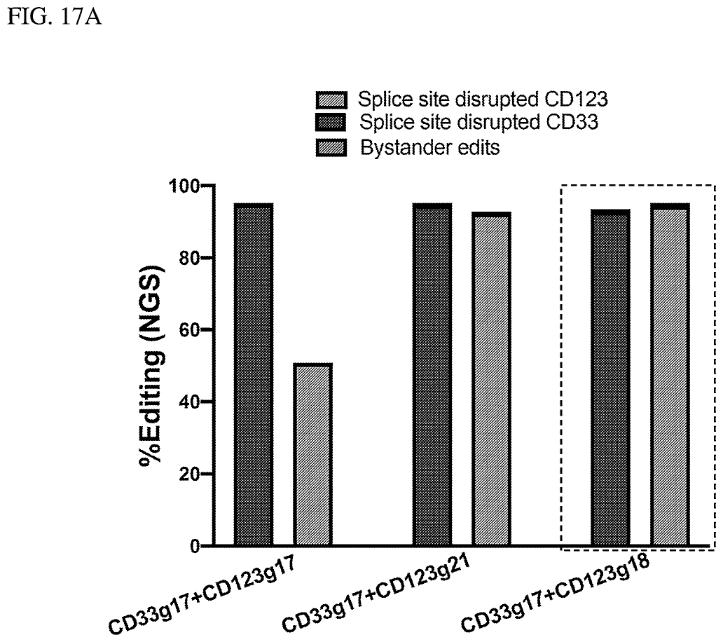

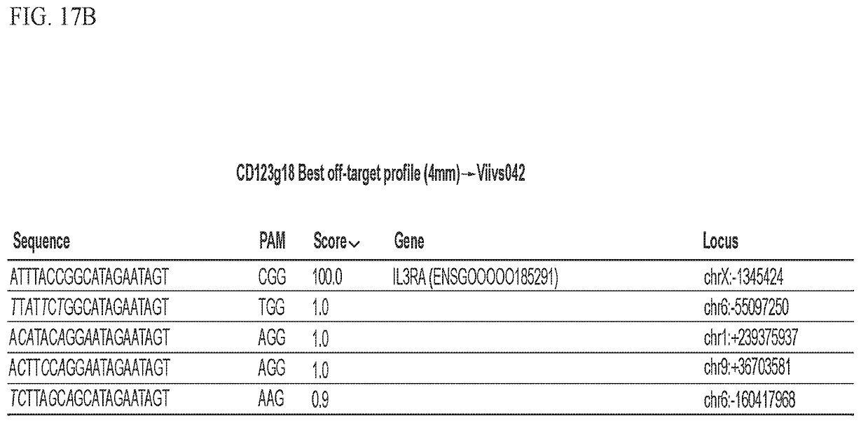

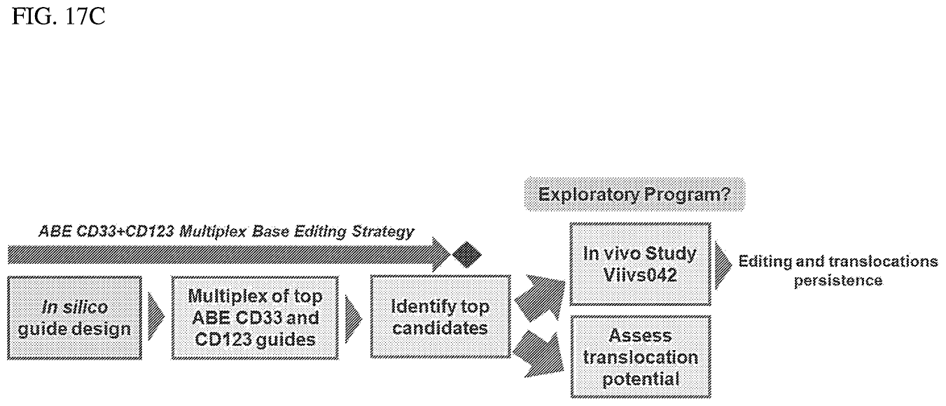

CD34+ hematopoietic stem and progenitor cells (HSPCs) from one healthy donor was thawed and cultured in maintenance media (SFEM+Flt3, SCF, TPO). Two days post-thaw, CBE or ABE editing was performed. For each condition, 8e5 cells were electroporated with 9 μg of CBE or ABE mRNA and 4.7 μM of guide-RNA. N1-methylpseudouridine-modified ABE8.20m mRNA was used. Additional control conditions were included. Guide-control samples received a non-targeting guide-RNA with ABE enzyme and underwent electroporation. Mock electroporation sample did not receive any enzyme or guide-RNA, but underwent electroporation. All conditions were then cultured in maintenance media (SFEM+Flt3, SCF, TPO) for five days post-electroporation. Cells for gDNA were harvested five days post-electroporation and next-generation sequencing (NGS) was performed to measure DNA editing of CBE and ABE guides. Flow cytometry was performed at five days post-electroporation to measure surface protein expression of CD33 in guide-edited and control samples. FIG. 17A shows comboplexing-simultaneous delivery of adenine base editor (ABE) and gRNA targeting CD33 and CD123 (e.g., CD33g17 and CD123g18, respectively) allows for about 90% on-target editing efficiency in CD123. In this experiment using ABE, there were no detectable bystander edits, only intended on-target splice site disruption for all targets graphed (FIG. 17A). As used herein, “bystander edits” refers to editing that occurs within the editing window (i.e., guide protospacer) at a nucleotide other than the targeted ‘A’ for ABE.

FIG. 17B shows off-target profile of adenine base editing with CD123g18. The CD123g18 off-target profile outlines potential targeted sites using in silico predicted homology to all sites in the human genome. This off-target prediction pipeline takes into consideration mismatches and gaps in the guide sequence and then gives a relative off-target score (e.g., the lower the score, the less potential activity). Those sites are mapped and recorded in the “Locus” column of the table. Out of the five listed sites, the first is the on-target site (score 100) and the following four are very low (score 1), thus displaying a more favorable off-target prediction for CD123g18.

FIG. 17C shows a schematic of the experimental design for multiplex editing of CD33 and CD123 performed using different CD33 and CD123 guide RNAs in combination with an adenine base editor (ABE).

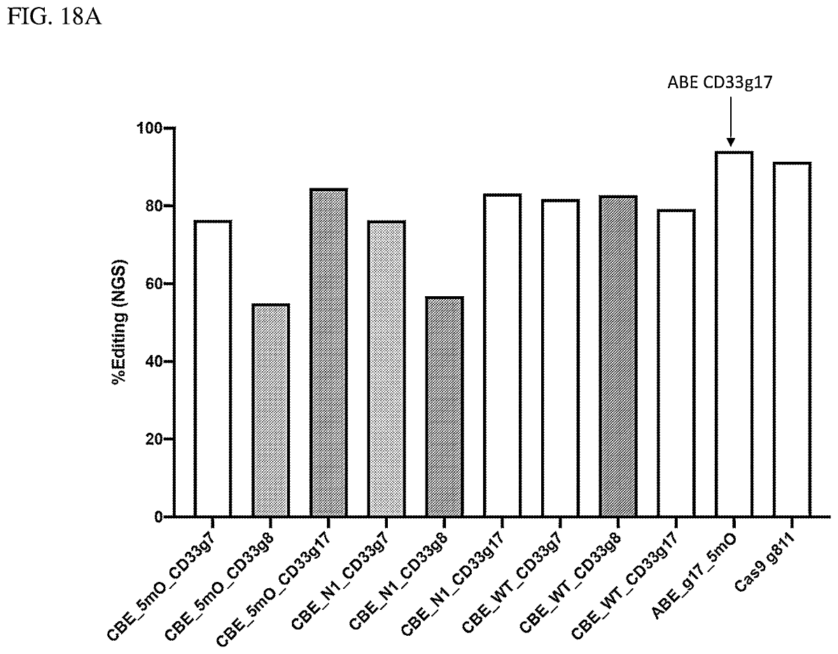

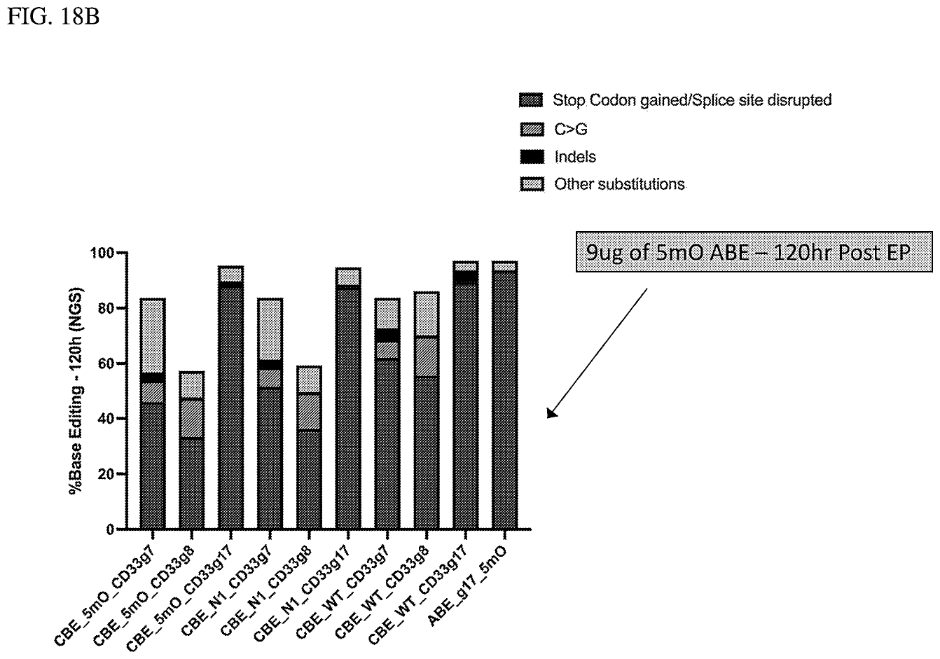

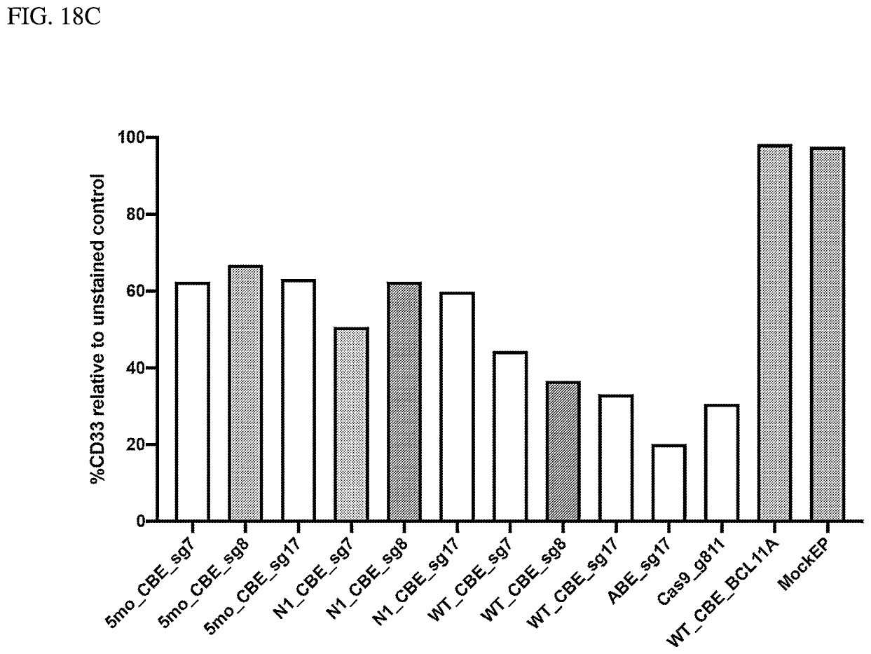

FIGS. 18A-18C show data from the cytosine base editor (CBE) screening efforts. Here each CD33 guide (e.g., CD33g7, CD33g8, and CD33g17) target was edited, as a single, with different modified CBE (5-methoxyuridine (5-mO), N1-methylpseudouridine (N1), or wild-type (WT)) to evaluate the CD33 guide/CBE pair in terms of on-target editing and protein knockout, as compared to ABE editing and protein knockout.

FIG. 18A shows on-target editing efficiency of single base edited cells for CD33 compared to Cas9 control edited cells. Base editing was performed using different CD33 guide RNAs (e.g., CD33g7, CD33g8, and CD33g17) in combination with an adenine base editor (ABE) or a cytosine base editor (CBE). These data demonstrate that the combination of adenine base editor (ABE) and CD33g17 resulted in about 90% on-target editing efficiency at 120 hours post electroporation of the CD34+ HSPCs with 9 μq of 5-methoxyuridine-modified mRNA encoding the ABE.

FIG. 18B shows on-target editing efficiency of single base edited cells for CD33 performed using different CD33 guide RNAs (e.g., CD33g7, CD33g8, and CD33g17) in combination with an ABE or a CBE. These data demonstrate that the combination of ABE and CD33g17 resulted in substantially all edits creating a substitution that would disrupt splicing at 120 hours post electroporation of the CD34+ HSPCs with 5-methoxyuridine-modified mRNA encoding the ABE.

FIG. 18C shows CD33 surface protein expression in edited and unedited CD34+ HSPCs 120 hours post-EP. These data demonstrate that the combination of ABE and CD33g17 resulted in a strong loss of CD33 surface protein expression compared to unedited (MockEP) at 120 hours post electroporation of the CD34+ HSPCs with 9 μq of 5-methoxyuridine-modified mRNA encoding the ABE.

### Example 7: ABE CD33g17 Included as Arm in First BE Multiplex Experiment

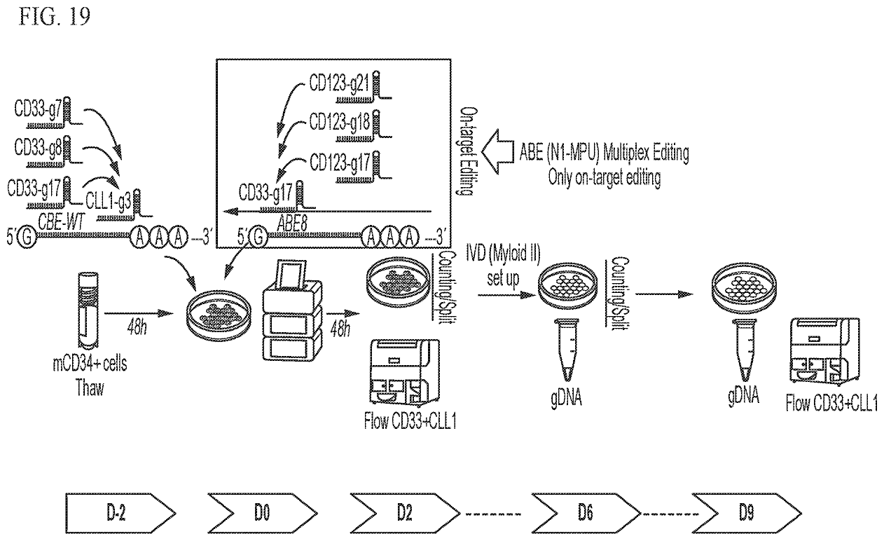

CD34+ hematopoietic stem and progenitor cells (HSPCs) from one healthy donor was thawed and cultured in maintenance media (SFEM+Flt3, SCF, TPO). Two days post-thaw, ABE editing was performed. For each ABE multiplex editing condition, 8e5 cells were electroporated with 9 μg of N1-Methylpseudouridine ABE8.20m mRNA and 4.7 μM of ABE CD33g17 guide-RNA in combination with different ABE CD123 guides (e.g., sg17, sg18, and sg21) to determine if N1-Methylpseudouridine ABE8.20m can silence CD33 and CD123 simultaneously in CD34+ cells. FIG. 19 illustrates the experimental design. This experiment demonstrates, for the first time, use of the ABE mRNA with the N1-methylpseudouridine chemical modification to increase stability of mRNA in the cell.

This experiment also included a Cas9 CD33g811/CLL1g6 multiplex editing condition to compare with the base editing multiplex editing of CD33 and CLL1.

Cells for gDNA were harvested five days post-electroporation and next-generation sequencing (NGS) was performed to measure DNA editing of ABE guides using the amplicon sequencing standard protocol. Flow cytometry was performed at five days post-electroporation to measure surface protein expression of CD33 and CD123 in guide-edited and control samples using a cytometer.

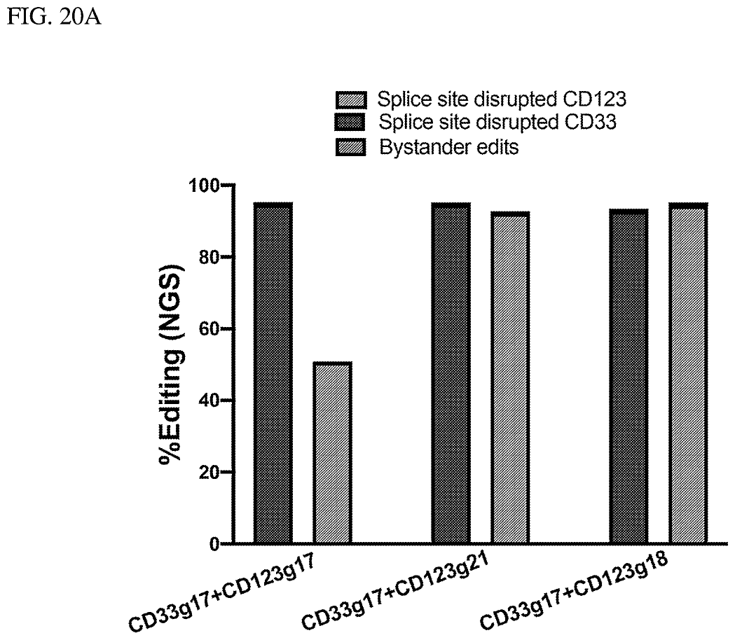

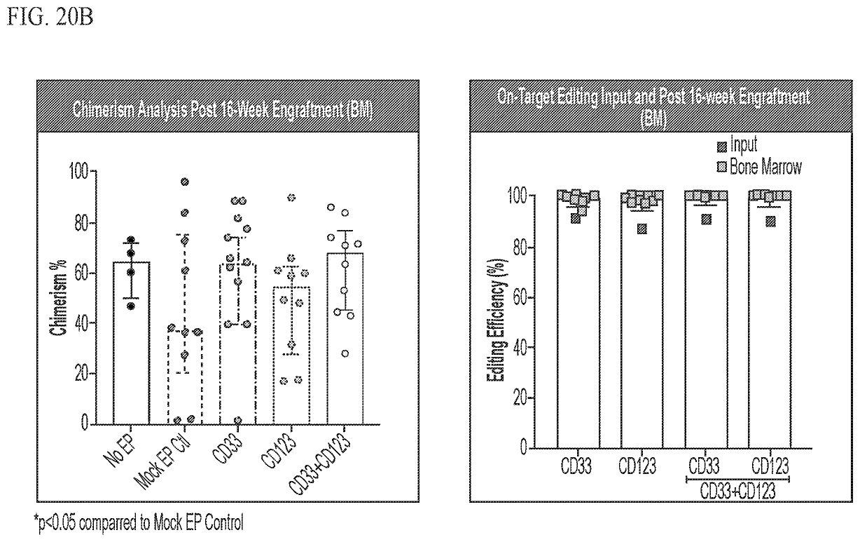

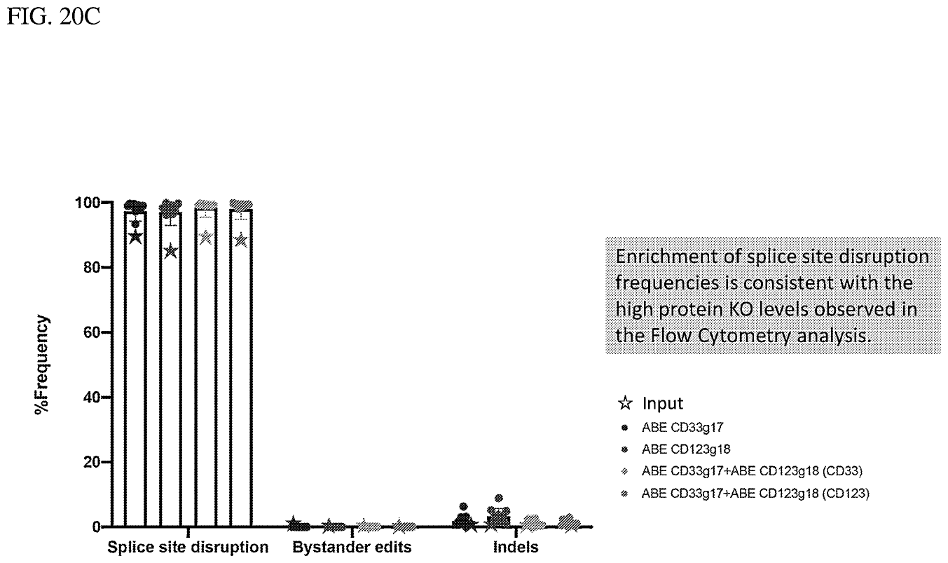

As shown in FIGS. 17A and 20A, comboplexing-simultaneous delivery of adenine base editor (ABE) and gRNA targeting CD33 and CD123 (e.g., CD33g17 and CD123g18, respectively) allows for about 90% on-target editing efficiency in CD123. FIG. 20B shows that multiplex deletion of myeloid antigens by base editing in human hematopoietic stem and progenitor cells (HSPCs) enables potential for next generation transplant for acute myeloid leukemia (AML) treatment. FIG. 20C shows that splice site disruption frequencies induced by ABE increased consistently across the different arms of the study.

### Example 8: Viivs042: Multiplex Base Editing In vivo Study

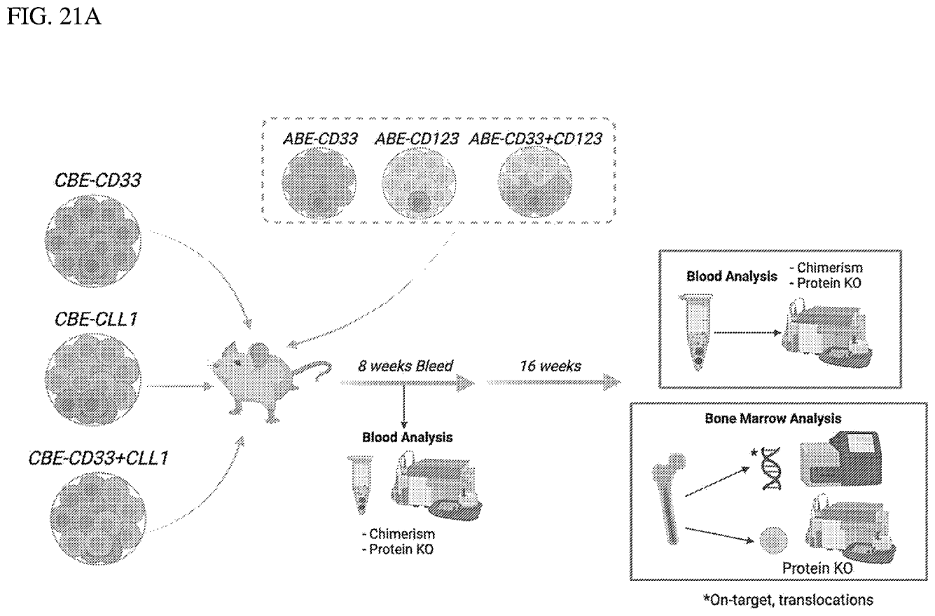

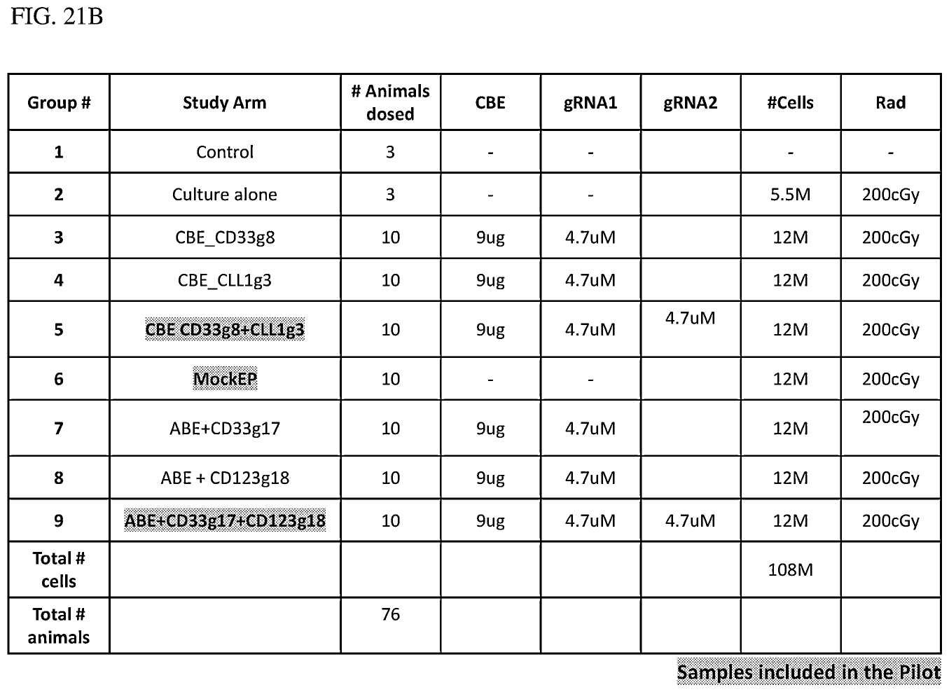

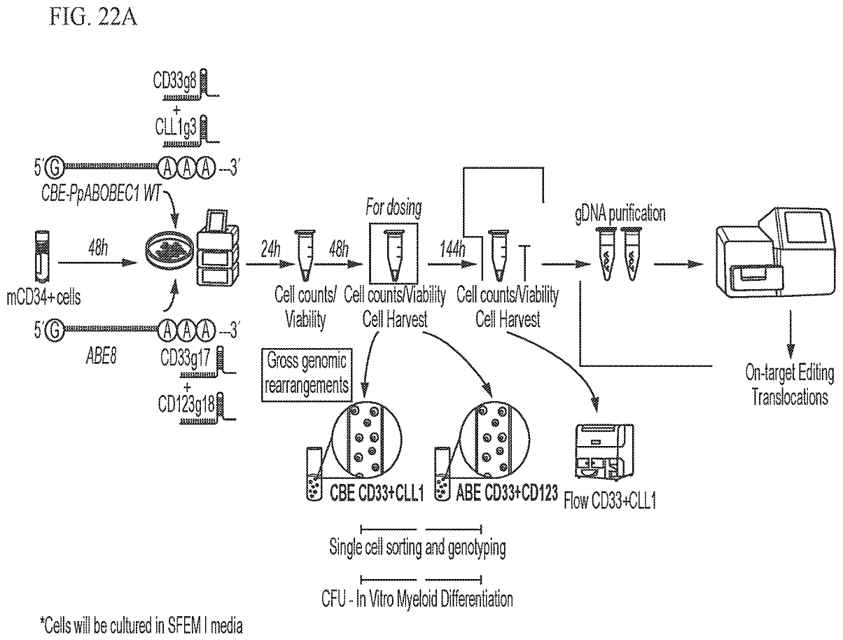

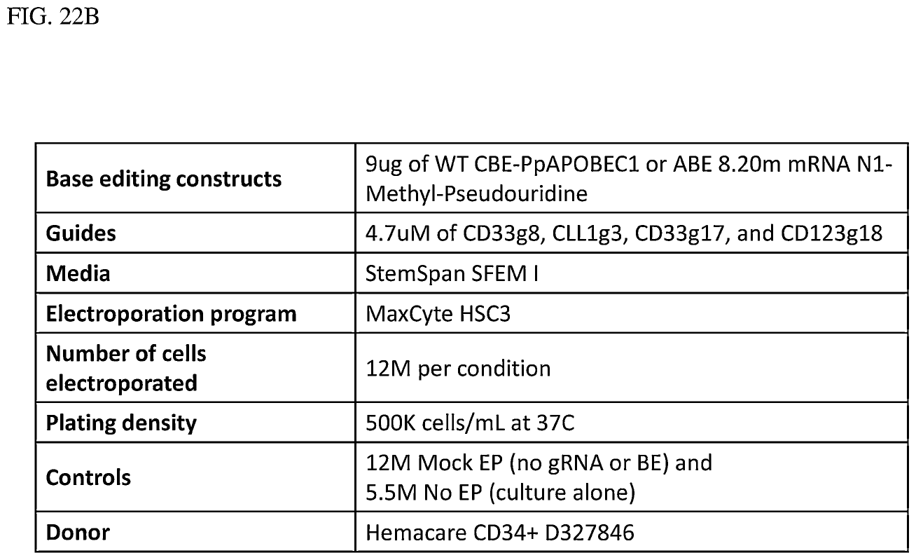

FIGS. 21A-21B and 22A-22B illustrates the experimental design and conditions to assess persistence of editing and long-term reconstitution of simultaneously CBE CD33+CLL1 and ABE CD33+CD123 multiplex edited CD34+ HSPCs in NSG mice.

Cells are thawed following the HSPC thaw protocol, and then allowed to rest in culture for 48 hours. Then, for the ABE portion, ABE CD33g17 is paired for multiplex editing with each of the ABE CD123g18. The 2 guides and ABE cargo is electroporated into the cells using the Maxcyte electroporation system. Cells are then cultured for 48 hours before harvesting for mouse dosing. A portion of the cells remained in culture to evaluate flow for protein knockout 144 hours post electroporation. Cells for gDNA for molecular analysis were collected 48 and 144 hours post electroporation.

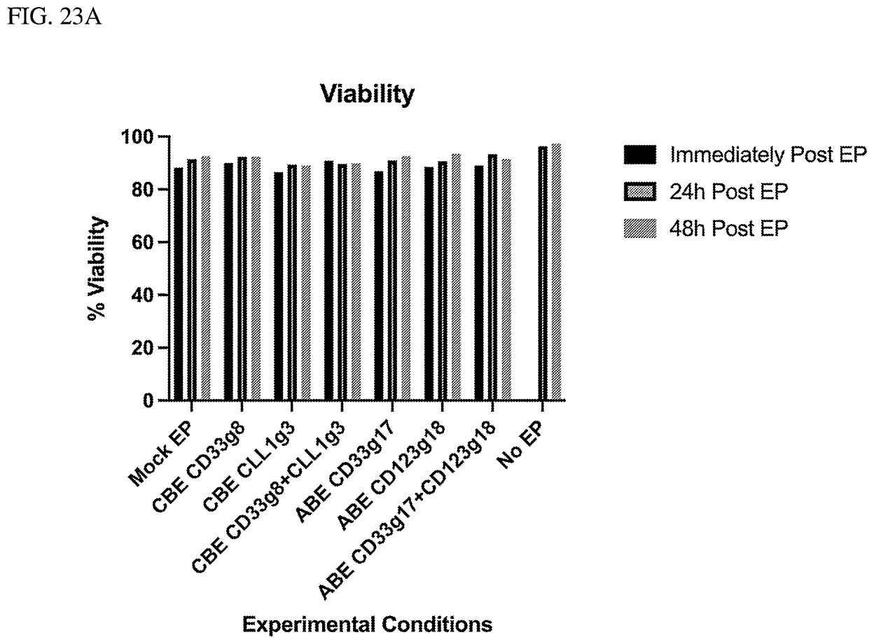

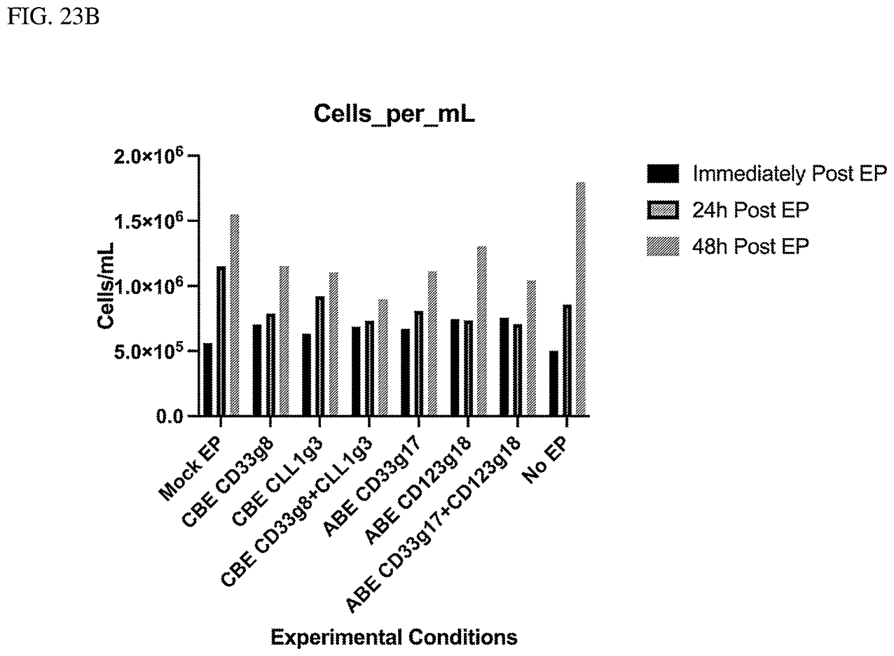

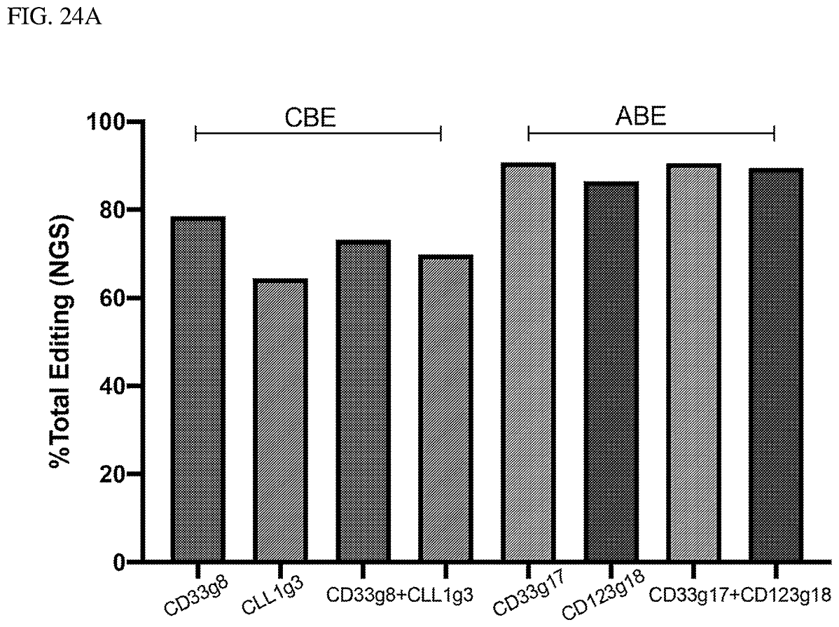

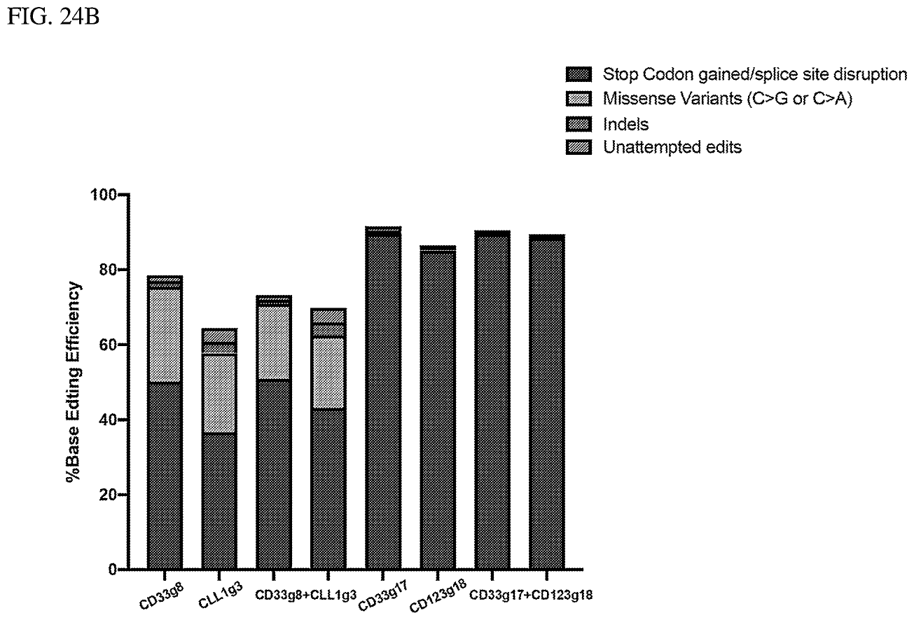

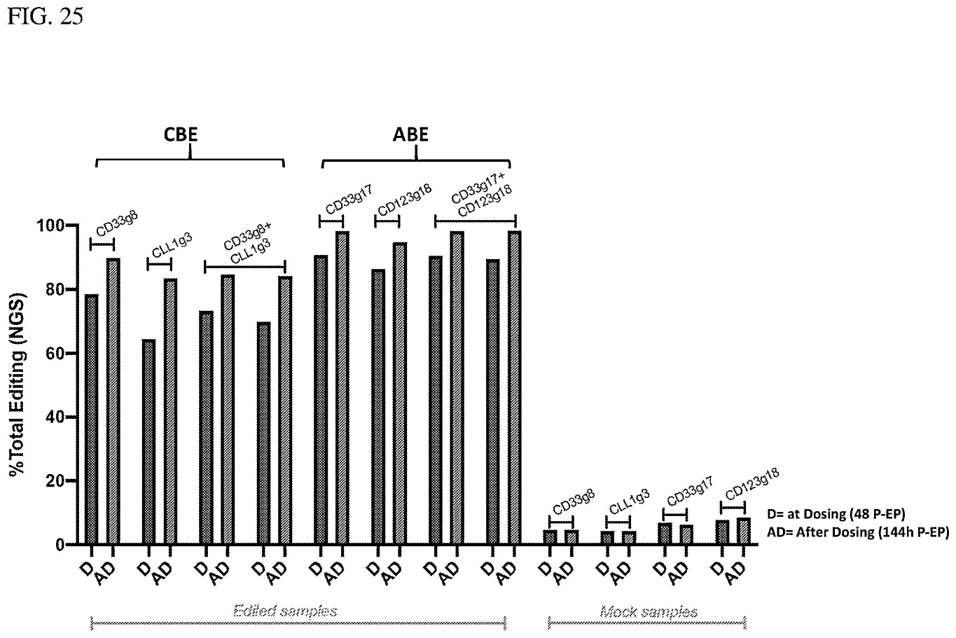

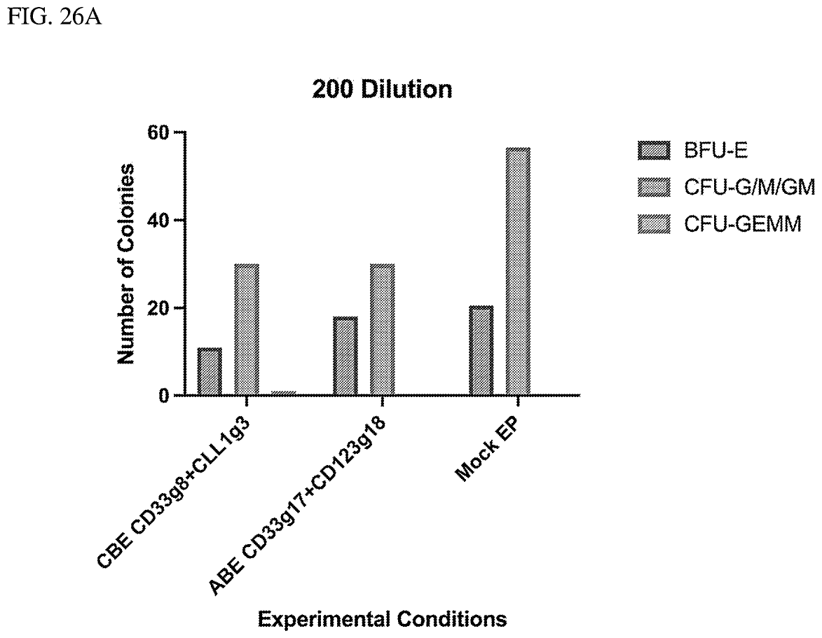

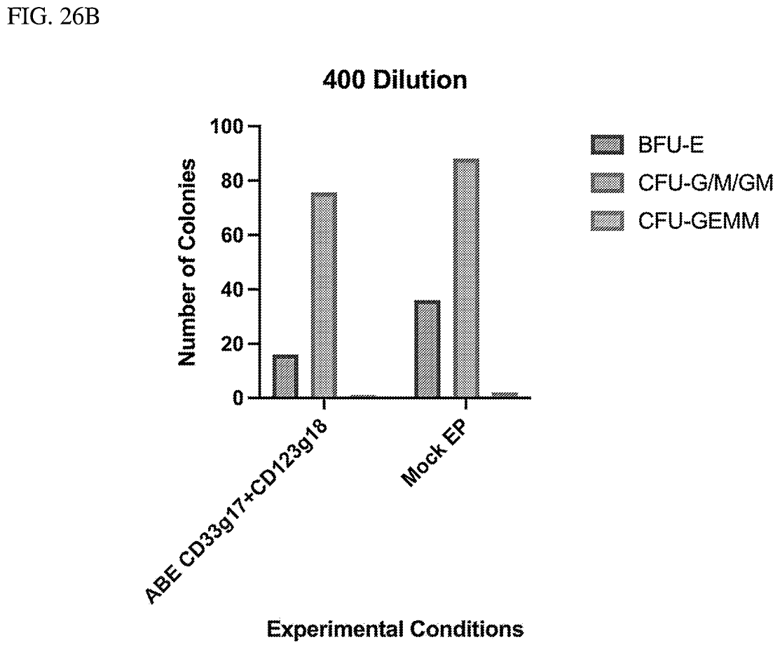

As shown in FIGS. 23A-23B, high cell viability of approximately 90% cell and similar cell counts were achieved in both the BE single and multiplex conditions. As shown in FIGS. 24A-24B, base editing efficiency in samples harvested 48 hour post electroporation (EP) for dosing showed expected alleles containing alleles with stop codons gain and splice sites disrupted. High total editing was also confirmed in all samples 48 hours post EP (dosed cells) and a slight increase 144 hour post EP (FIG. 25). FIGS. 26A-26B shows colony-forming unit (CFU) results at 200 dilution and at a 400 dilution, respectively.

### Example 9: 16 Weeks BM Data

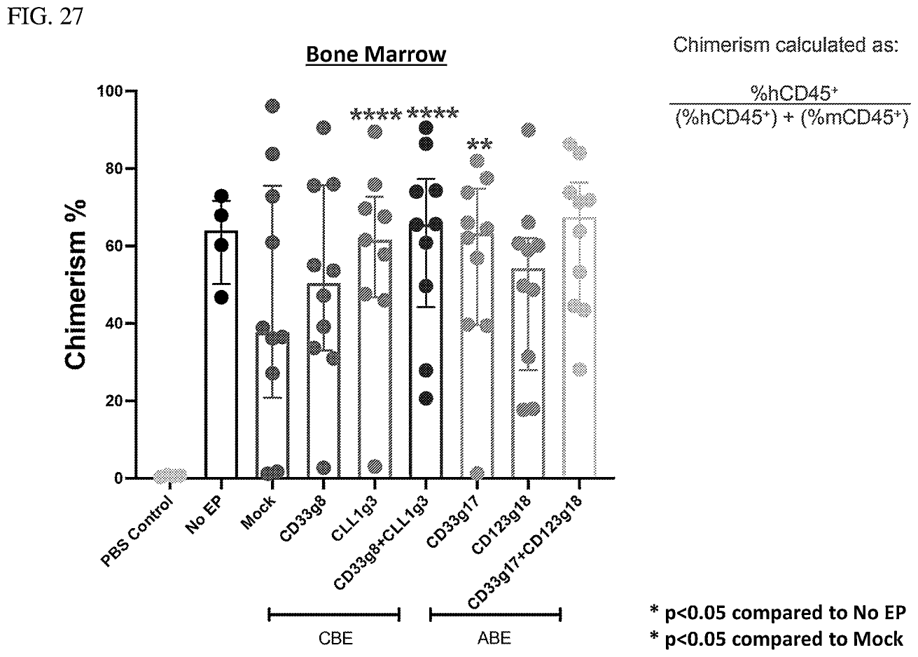

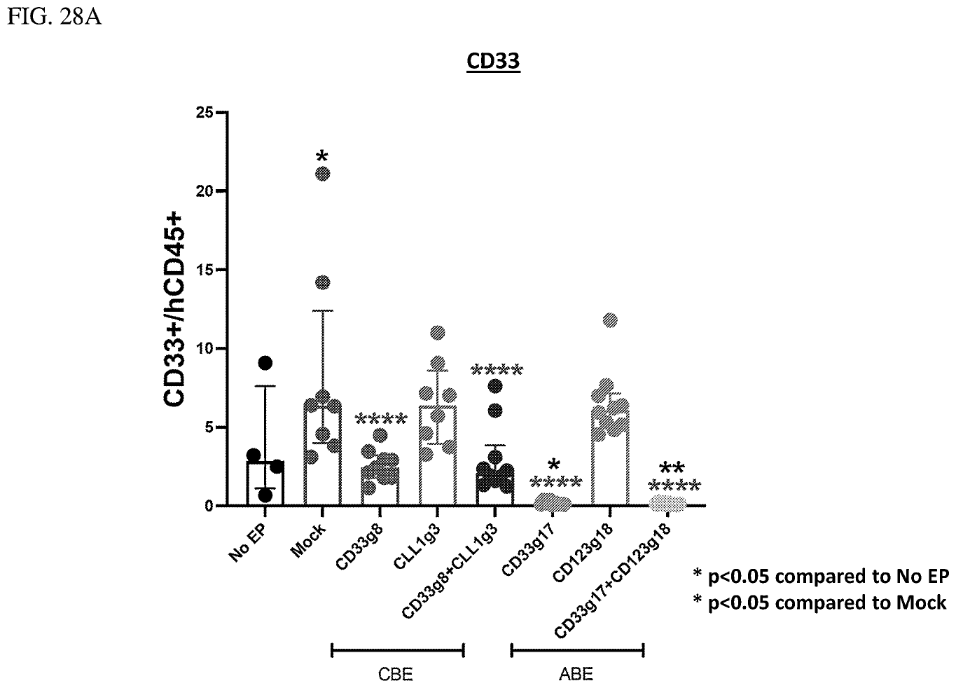

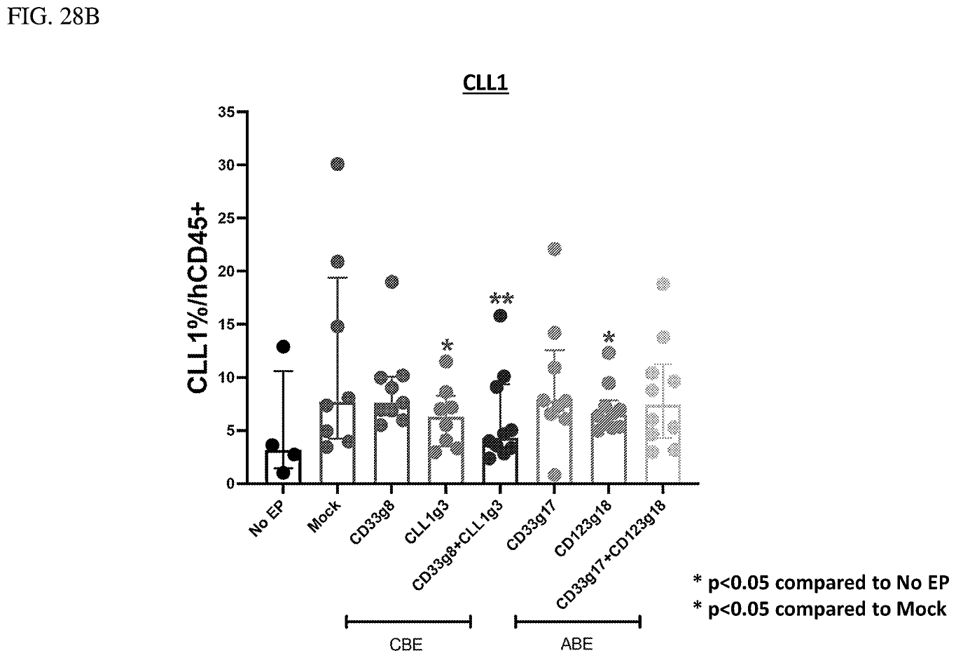

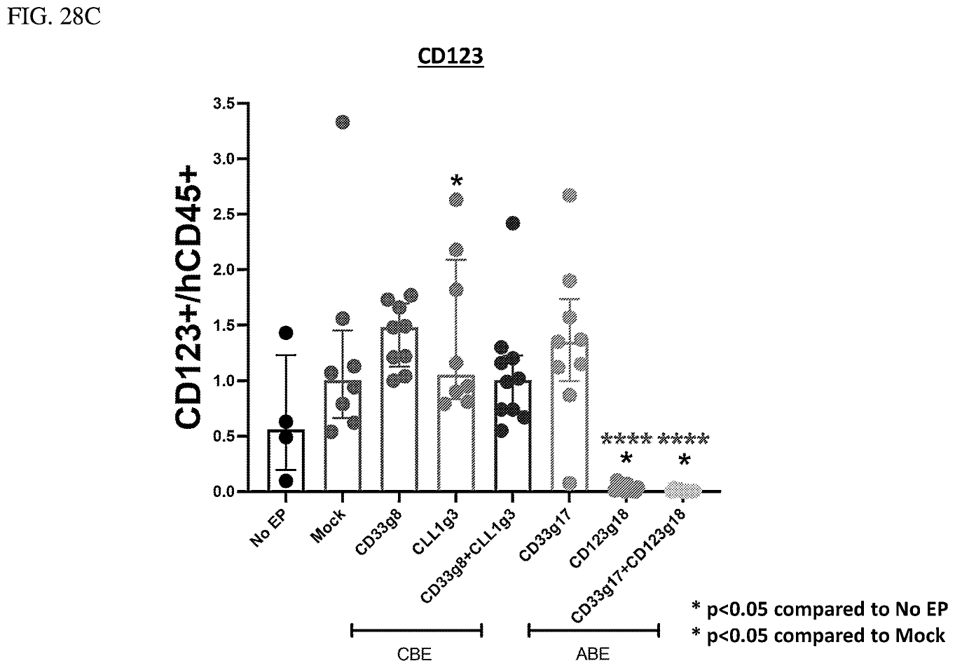

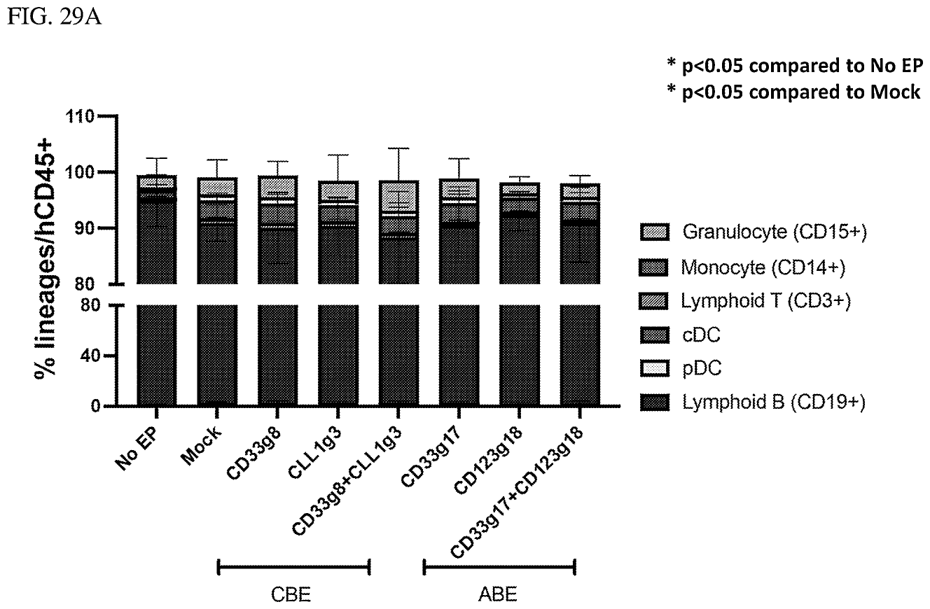

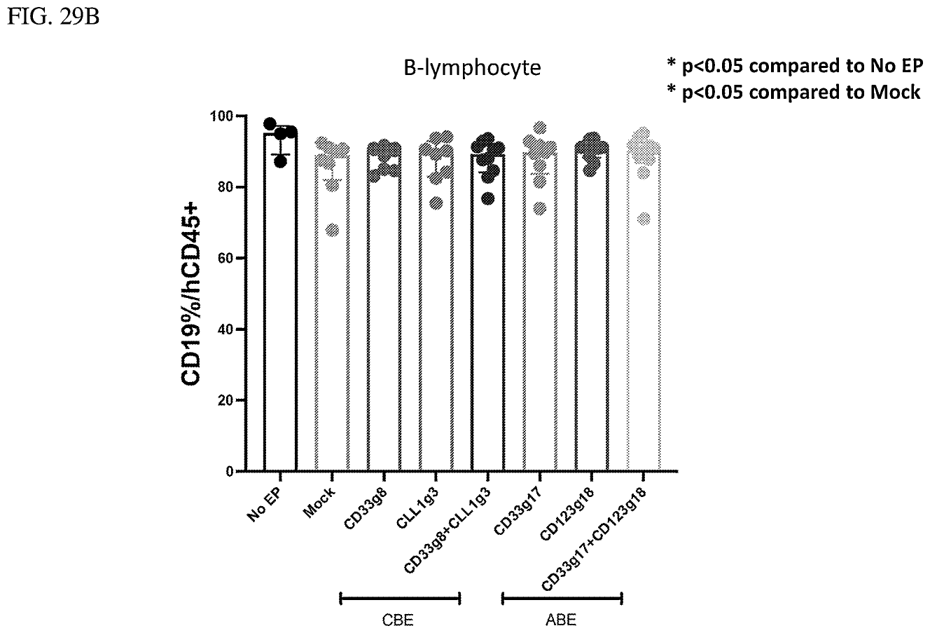

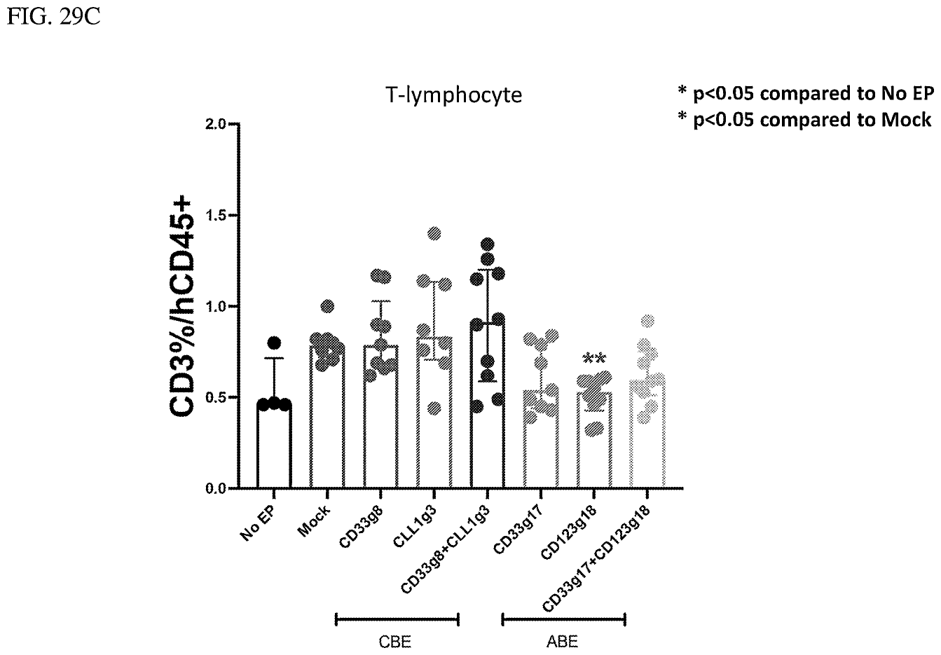

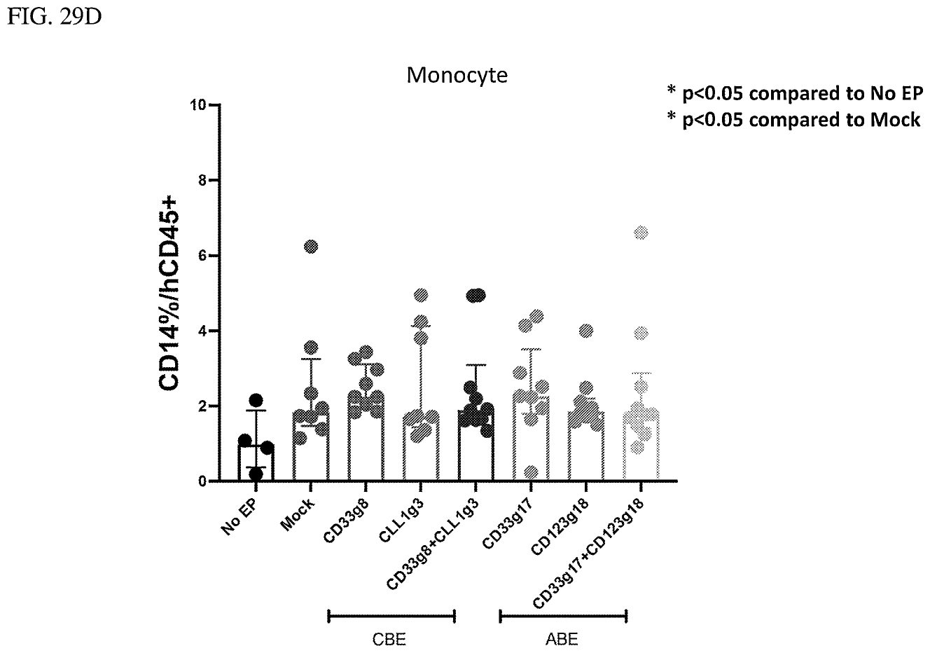

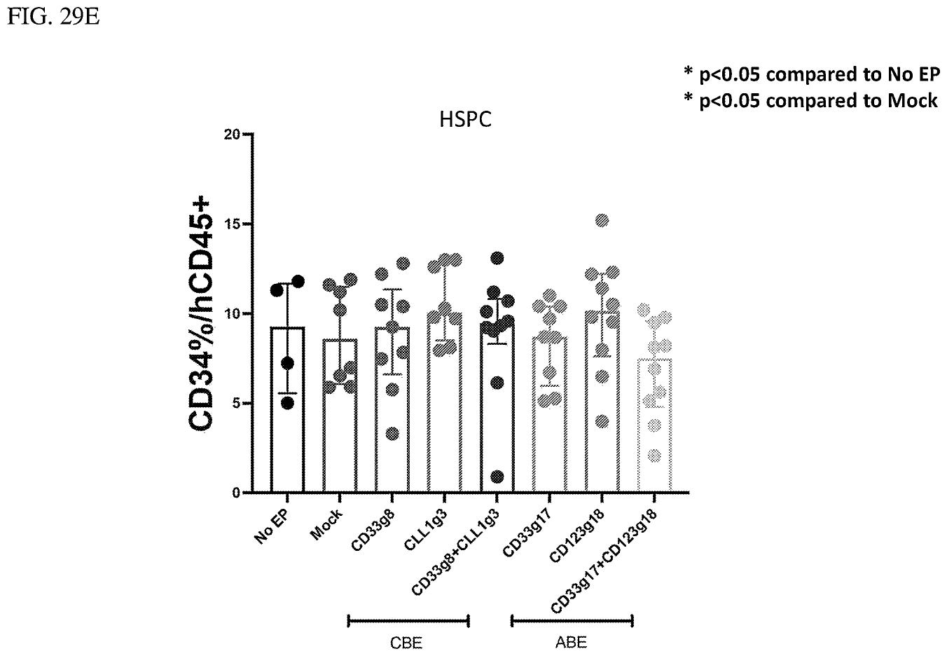

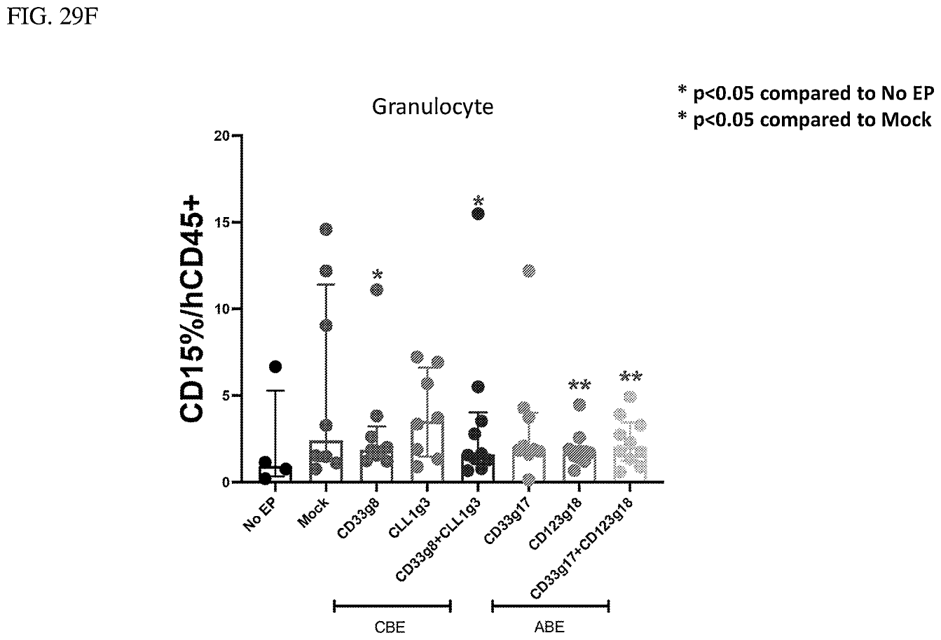

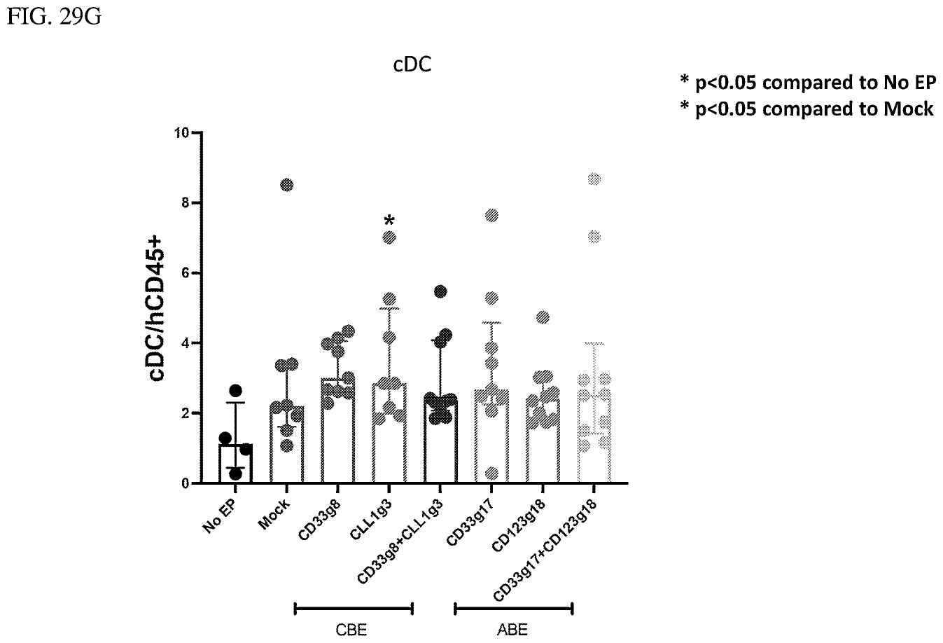

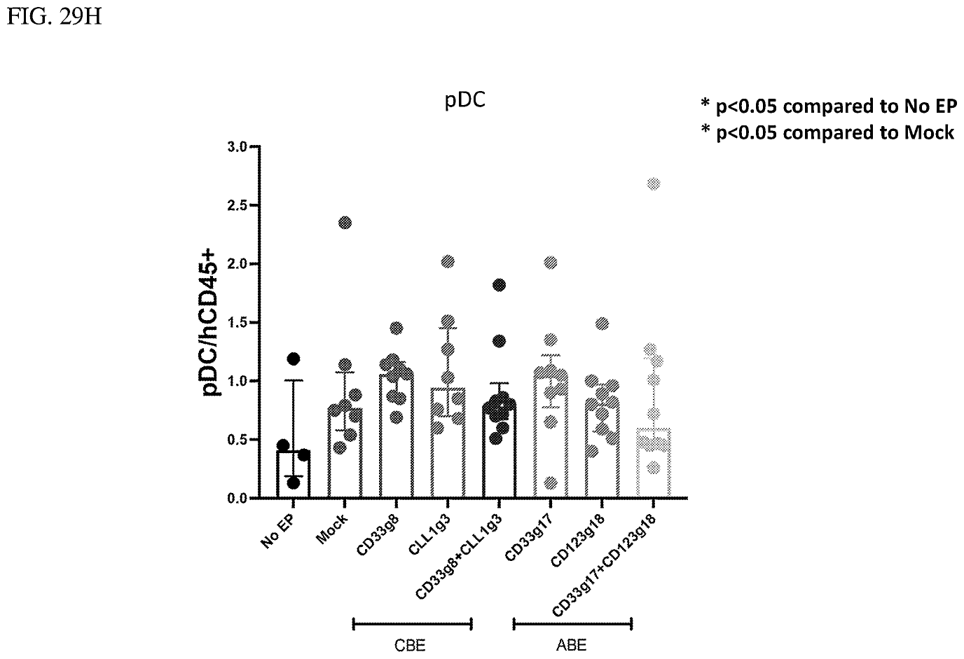

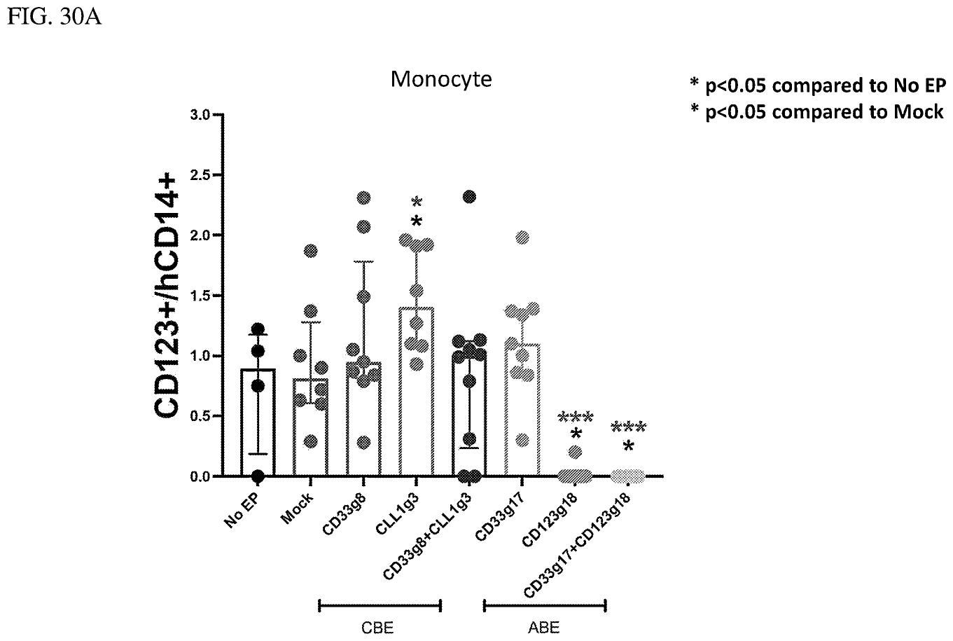

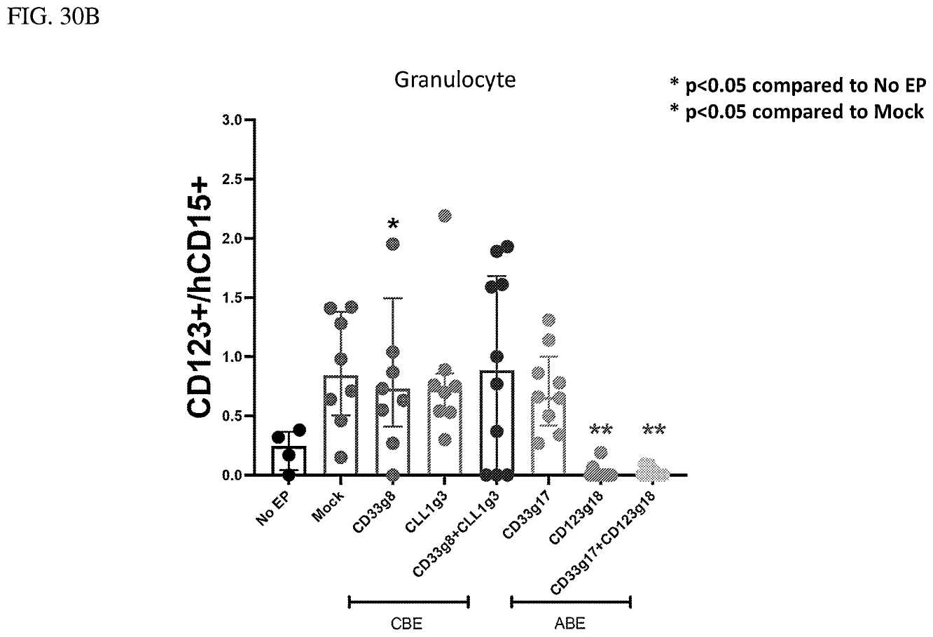

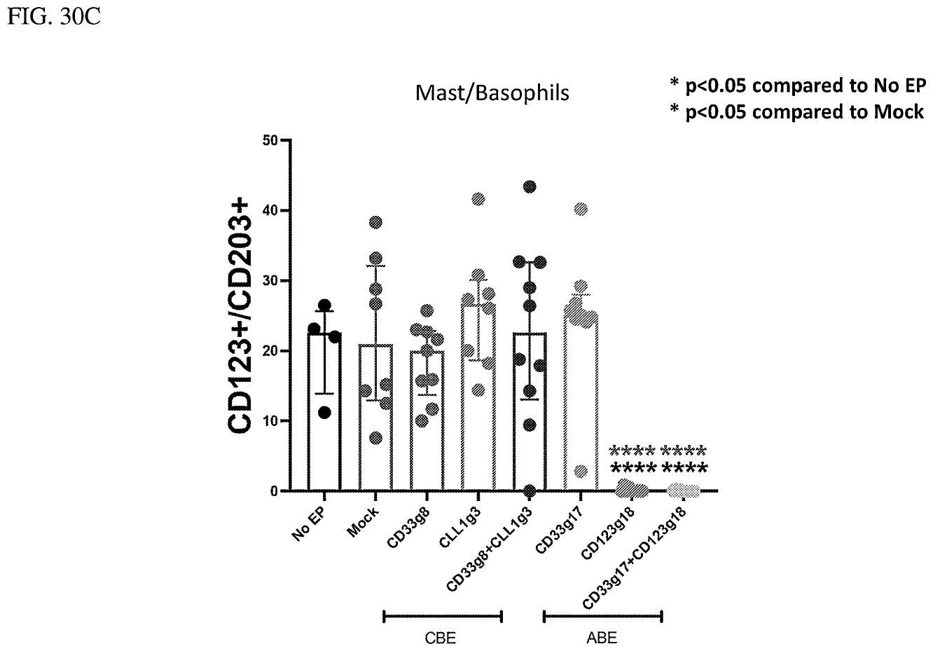

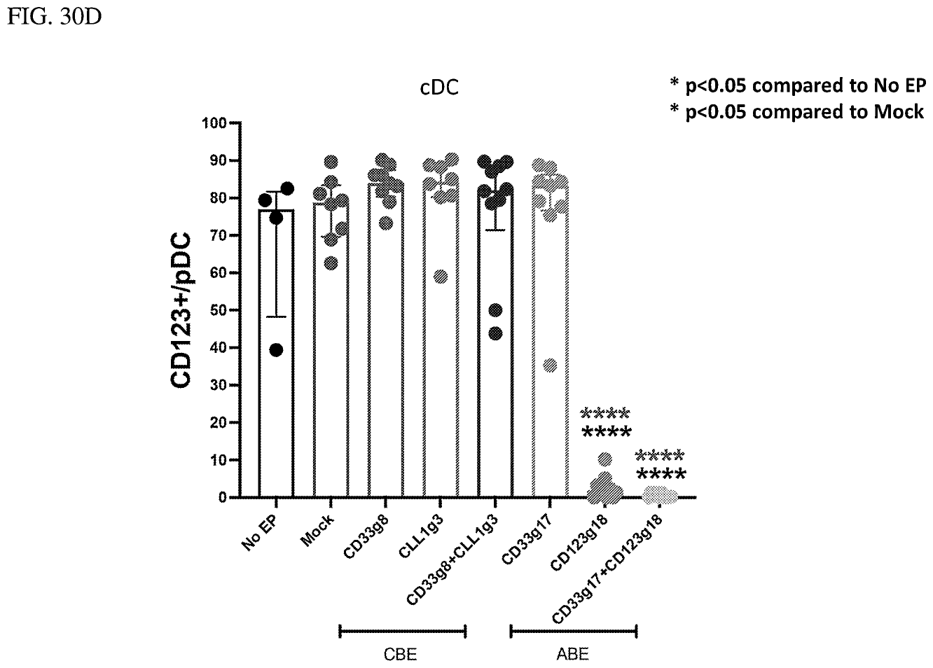

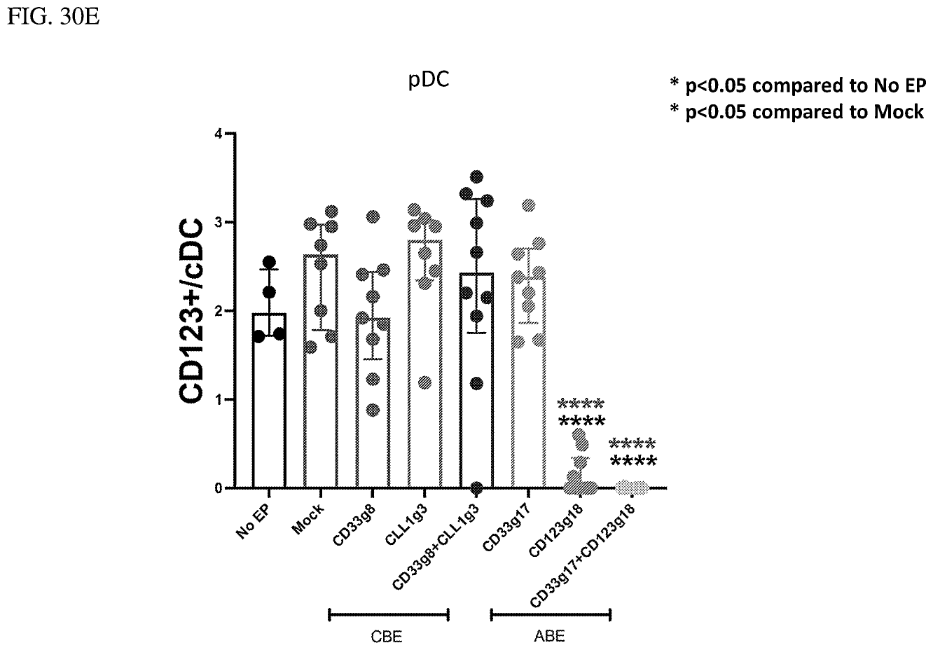

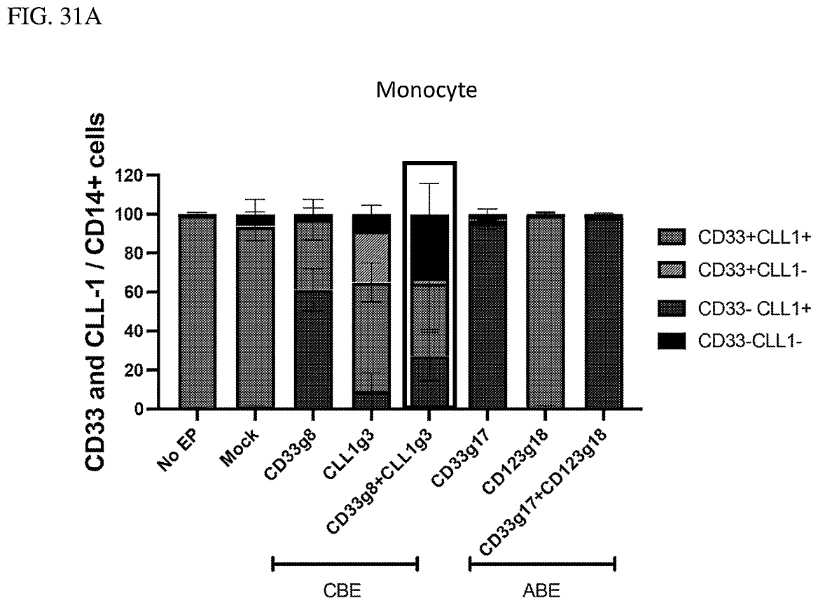

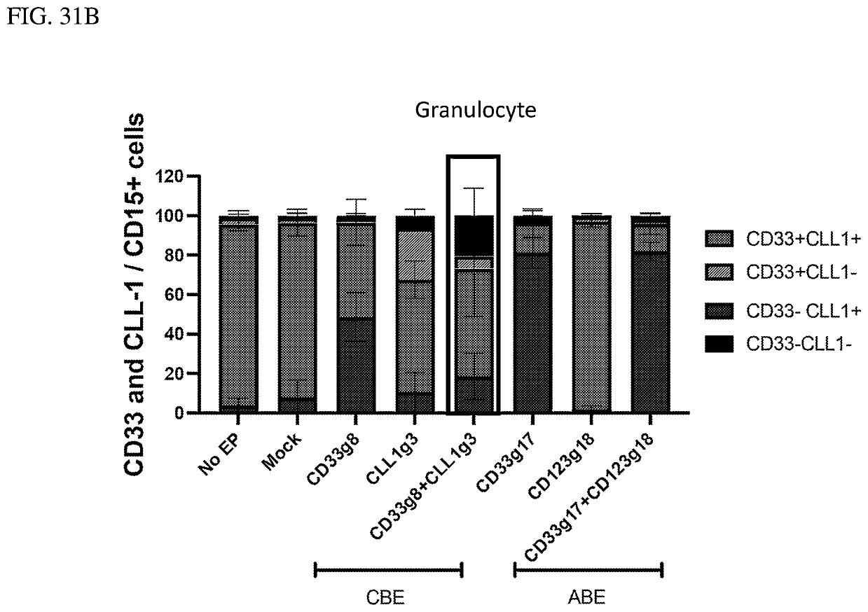

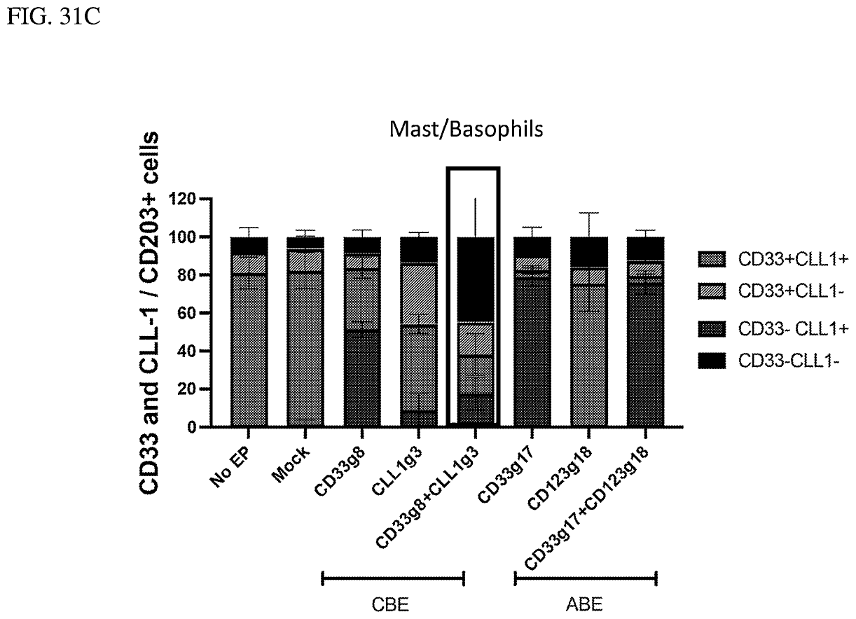

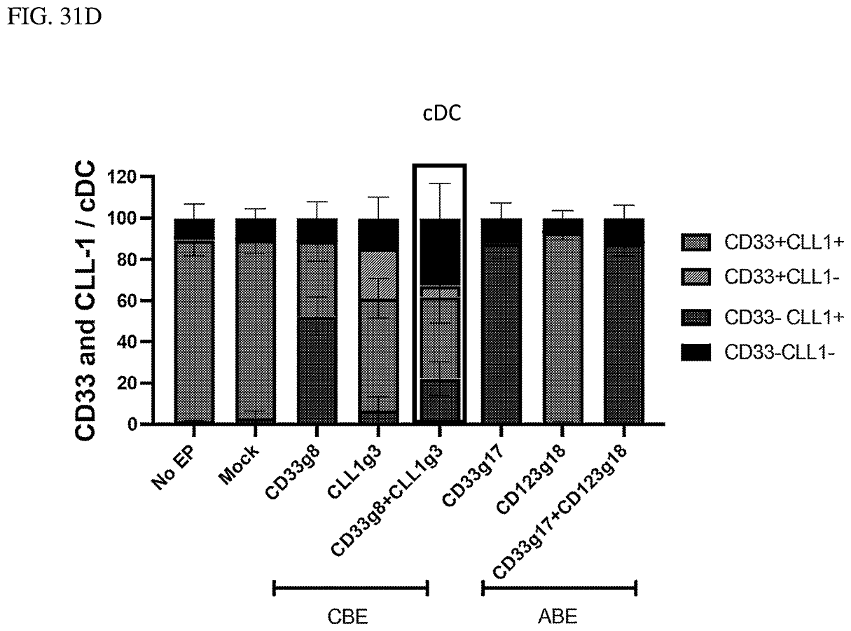

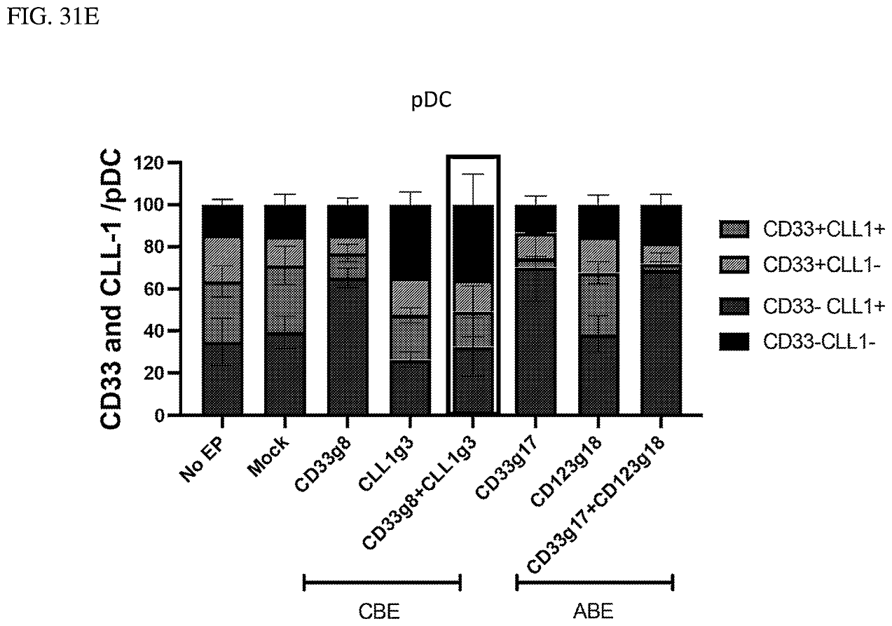

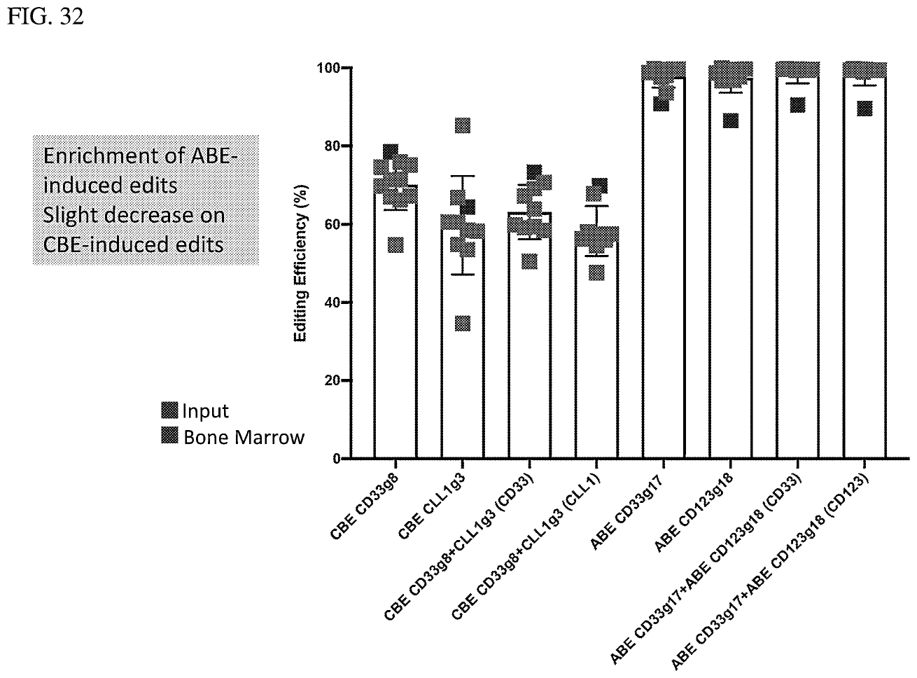

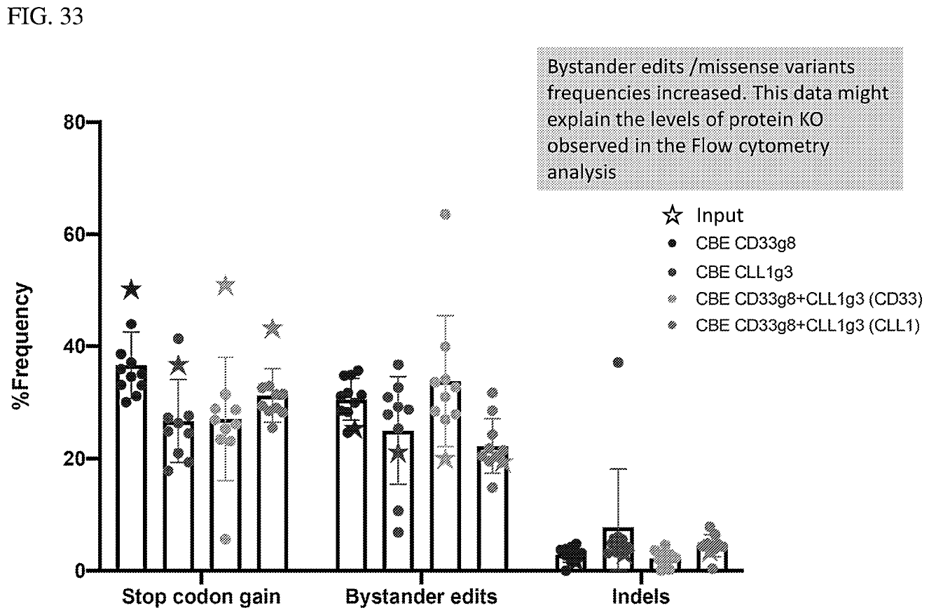

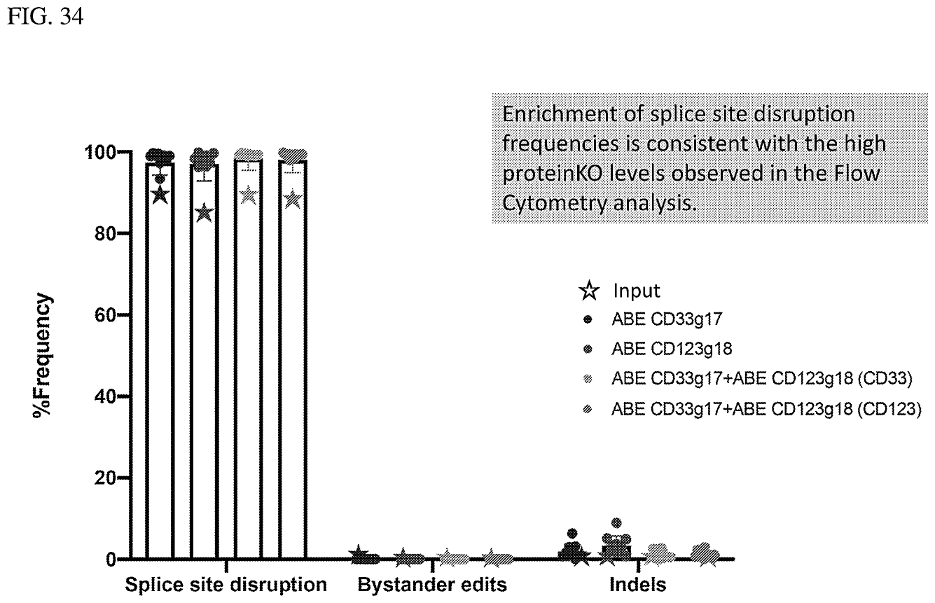

16 week bone marrow was harvested and cells were stained for specific surface protein to evaluate chimerism, surface protein knockout, lineage reconstitution of stem and progenitor cells, CD123 knockout is CD34+ subpopulations, and CLL1 knockout in subpopulations for the CBE portion of this experiment. As shown in FIG. 27, no impact was observed on in the chimerism of the edited groups post 16-week engraftment in bone marrow (BM). Post 16-week engraftment in bone marrow (BM), highly efficient knockout of CD33 (FIG. 28A), CLL-1 (FIG. 28B), and CD123 (FIG. 28C) was observed in edited groups with ABE. No effect was observed in lineage reconstitution in edited groups (FIGS. 29A-29H). FIG. 29A shows total lineage reconstitution in edited groups. FIGS. 29B-29H show lineage reconstitution in edited groups across different cell types, including: B-lymphocytes (FIG. 29B), T-lymphocytes (FIG. 29C), Monocytes (FIG. 29D), HSPCs (FIG. 29E), Granulocytes (FIG. 29F), cDCs (FIG. 29G), and pDCs (FIG. 29H). Post 16-week engraftment in bone marrow (BM), high levels of CD123 KO in myeloid subpopulations across different cell types, including: Monocytes (FIG. 30A), Granulocytes (FIG. 30B), Mast/Basophils (FIG. 30C), cDCs (FIG. 30D), and pDCs (FIG. 30E) was observed. However, low levels of double KO in myeloid subpopulations were observed due to low levels of CLL1 KO across different cell types, including: Monocytes (FIG. 31A), Granulocytes (FIG. 31B), Mast/Basophils (FIG. 31C), cDCs (FIG. 31D), and pDCs (FIG. 31E). As shown in FIG. 32, on-target editing analysis in Bone Marrow material across the different arms of the study confirm editing persistence. Additionally, stop codon frequencies induced by CBE slightly decreased consistently across the different arms of the study (FIG. 33), while splice site disruption frequencies induced by ABE increased consistently across the different arms of the study (FIG. 34).

### Example 10: CBE CD33/CLL-1 Scale-up Optimization

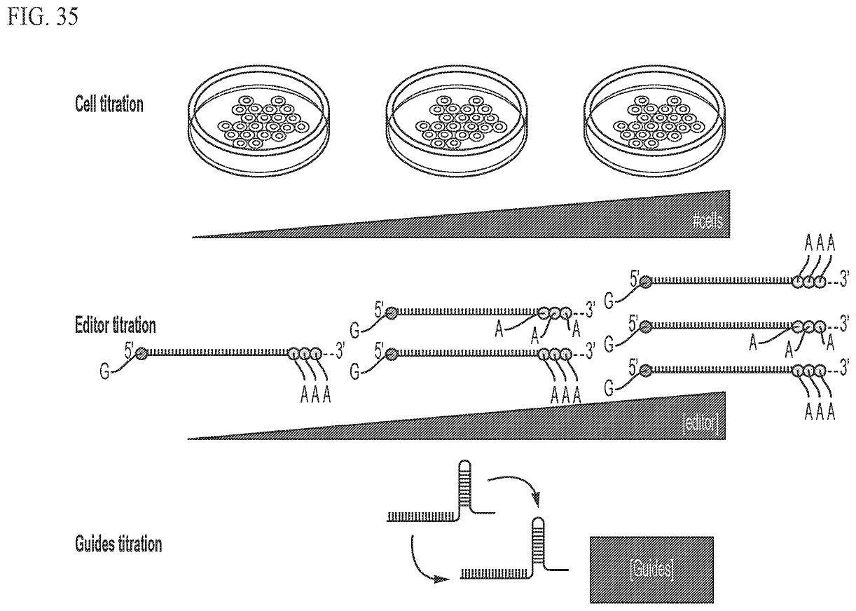

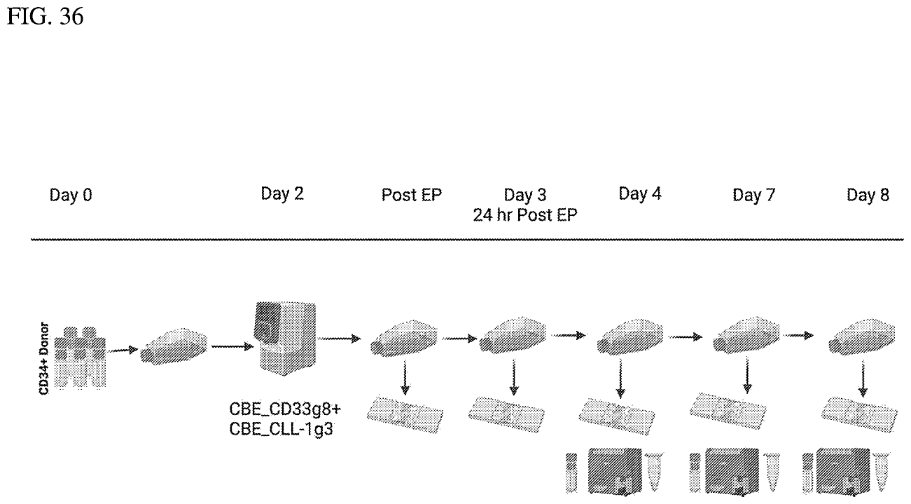

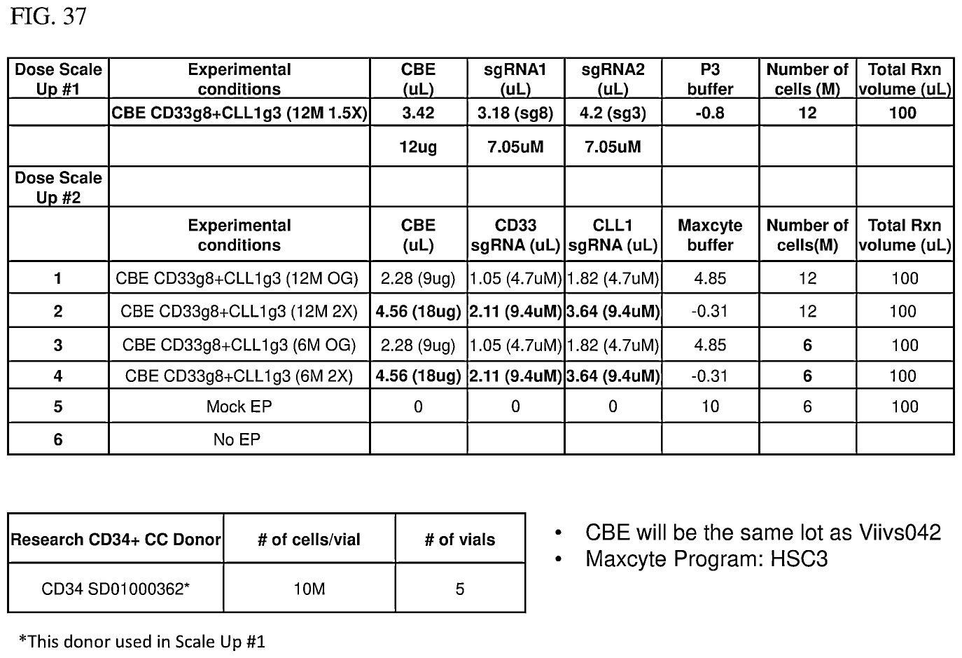

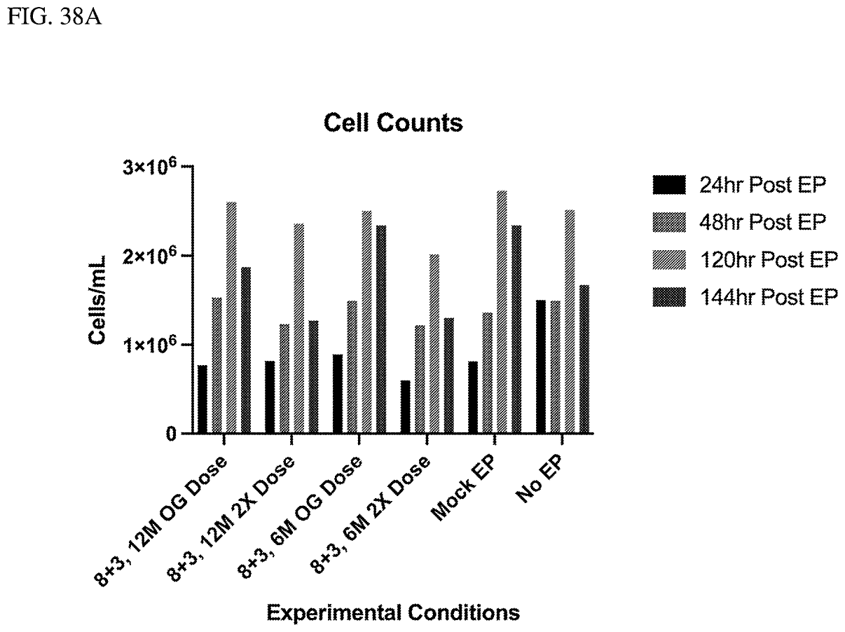

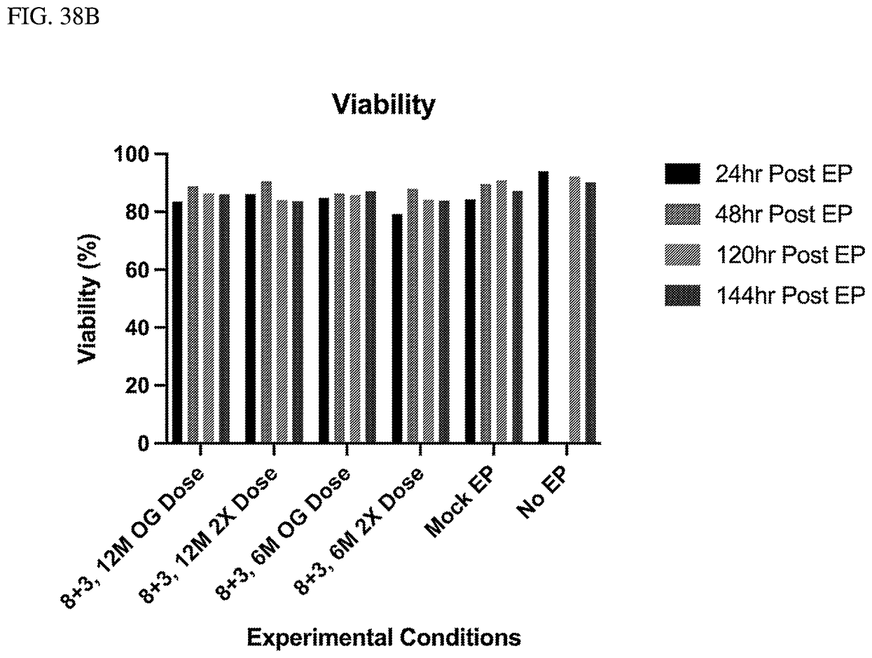

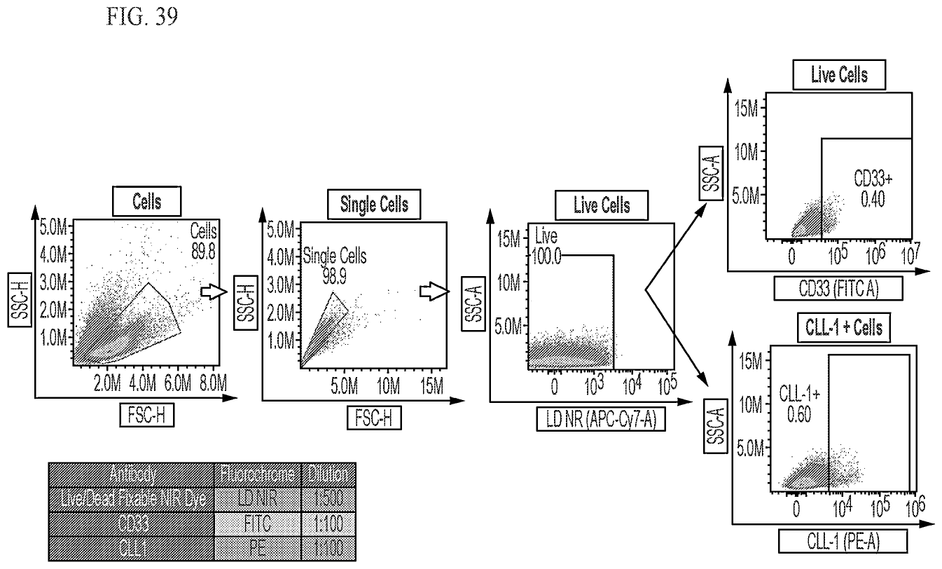

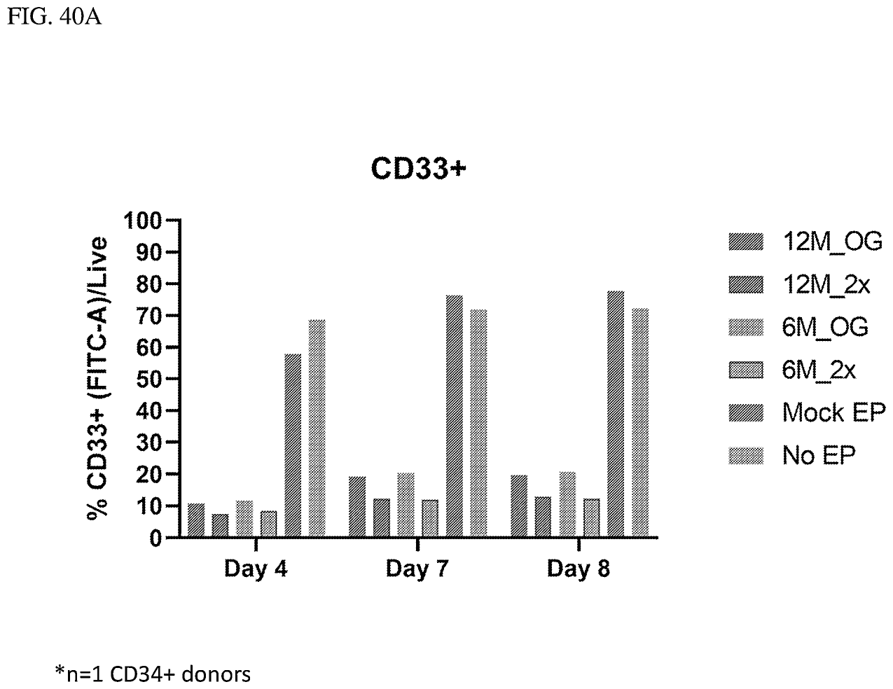

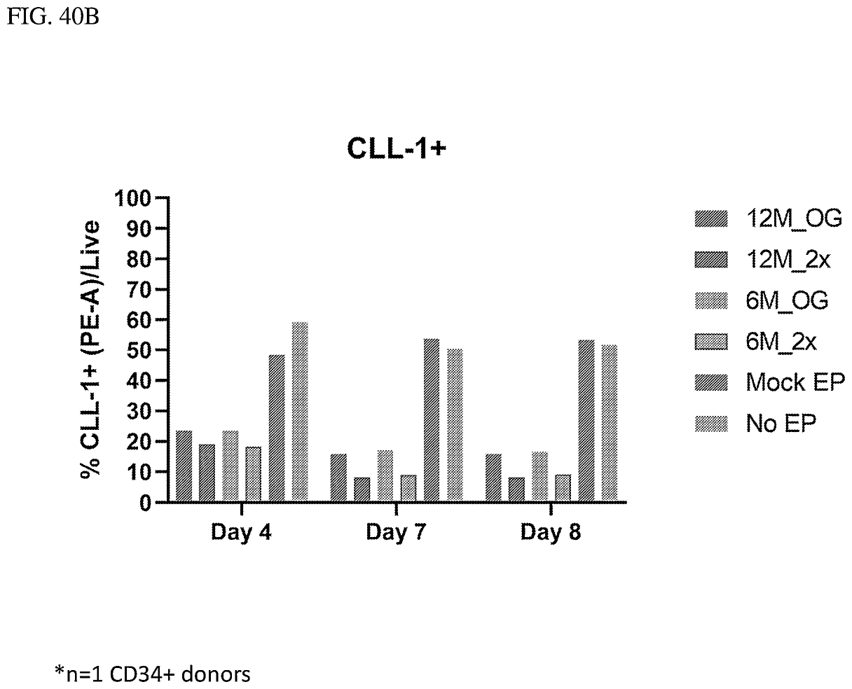

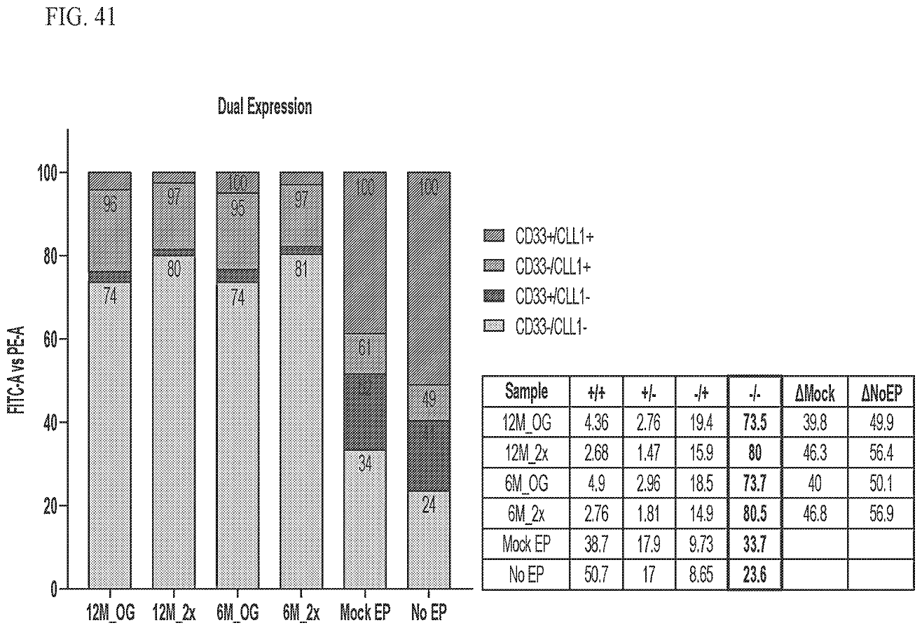

Multiplex edit CD34+ cells using CBEs with CD33g8+CLL1g3 at 2× the dose used with Viivs042 (the initial base editing in vivo study) to see if editing efficiency is increased. This experimental also included electroporation of 6M cells with the original Viivs042 dose and with 2× that dose, rather than the standard 12M cells, to determine if varying cell number affects editing efficiency. FIGS. 35-37 illustrates the experimental design and conditions assessed. As shown in FIGS. 38A-38B, shows cell counts and viability, respectively. Cells growth was slightly reduced in the 6M cell, 2× Dose condition. FIGS. 39 an 40A-40B shows flow gating strategy and results for CD33 and CLL-1. FIG. 41 shows dual knockout of CD33 and CLL-1.

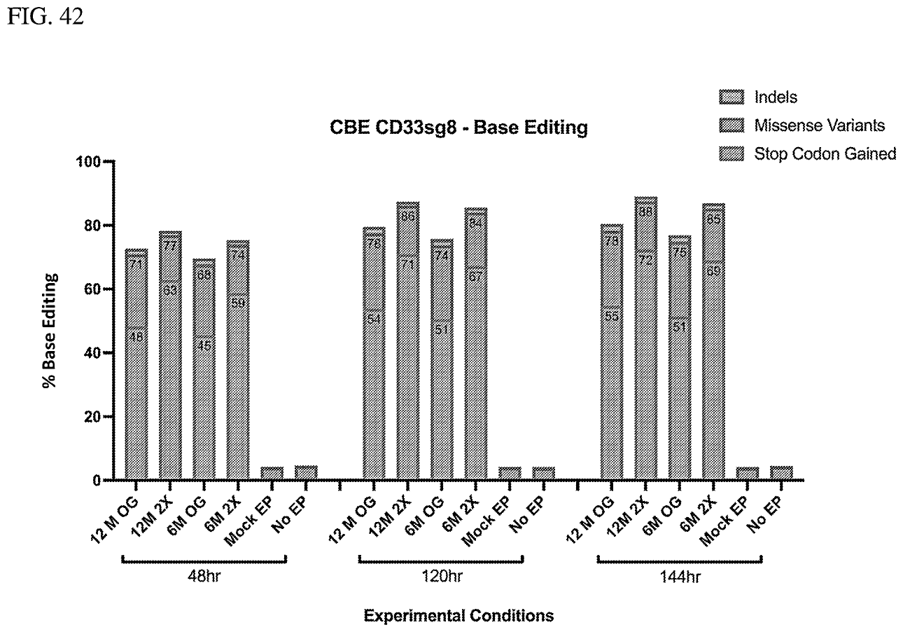

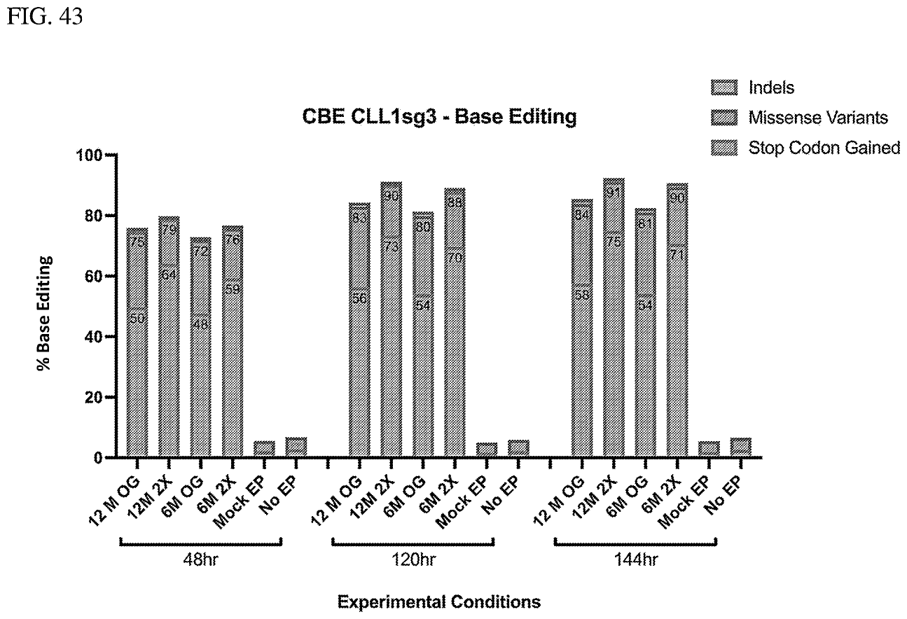

FIG. 42 shows that 2× dose results in higher frequency of alleles that result in premature stop codon formation for CBE CD33g8, while FIG. 43 shows that 2× dose results in higher frequency of alleles that result in premature stop codon formation for CBE CLL1g3.

### Example 11: CBE and ABE CD33/EMR2

One CD34+ donor was thawed and cultured in maintenance media (SFEM+Flt3, SCF, TPO). Two days post-thaw, ABE or CBE editing was performed. For each condition, 1e6 cells were electroporated with 9 μg of either ABE (N1-MPU ABE8.20m mRNA) or CBE (WT PpABOBEC1 mRNA) mRNA and 4.7 μM of guide-RNA. Additional control conditions were included. Guide-control samples received a non-targeting guide-RNA with Cas9, ABE or CBE enzyme, and underwent electroporation. Mock electroporation sample did not receive any enzyme or guide-RNA, but underwent electroporation. Two lead Cas9 guide conditions for knock-out comparison received EMR2 guide-329 and CD33 guide-811 and were electroporated with 15 μg SpCas9 enzyme and 15 μg Cas9 guide-RNA. All conditions were then cultured in maintenance media (SFEM+Flt3, SCF, TPO) for six days post-electroporation. Cell counts and viability were measured using the Nexcelom Cellometer and AOPI stain (1:2 dilution) at one, two and six days post-electroporation. Cells for gDNA were harvested at two and six days post-electroporation and rhAmpSeq (NGS) was performed to measure DNA editing of ABE, CBE or Cas9 guides. Flow cytometry was performed at six days post-electroporation to measure surface protein expression of CD33 and EMR2 in guide-edited and control samples using a cytometer.

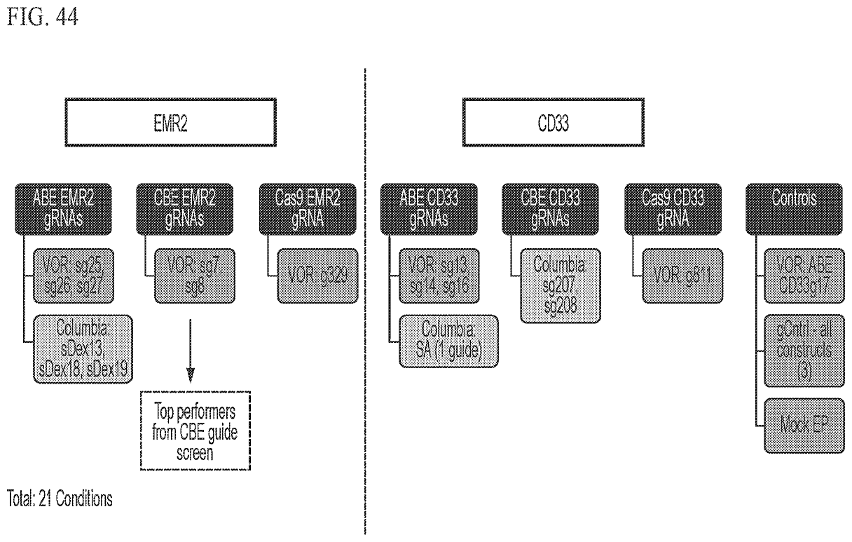

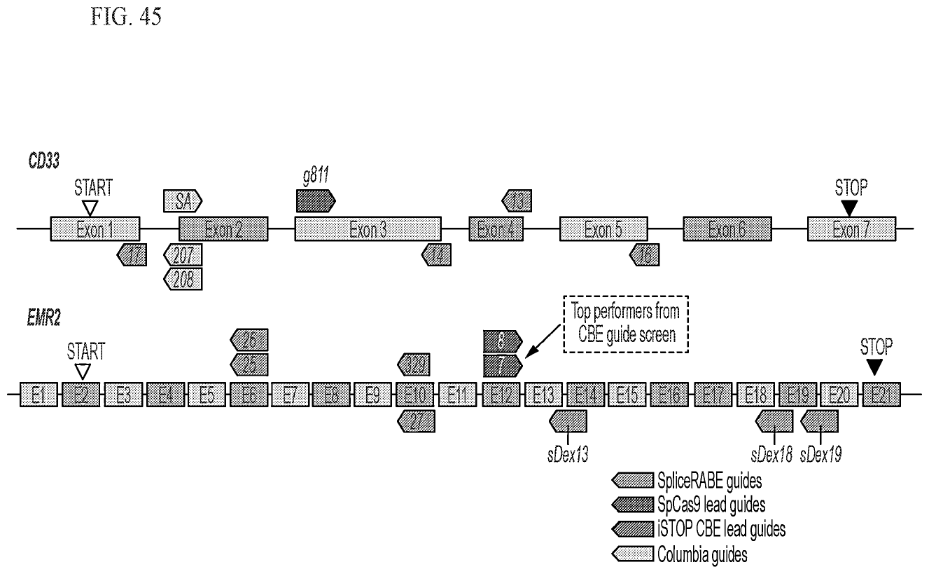

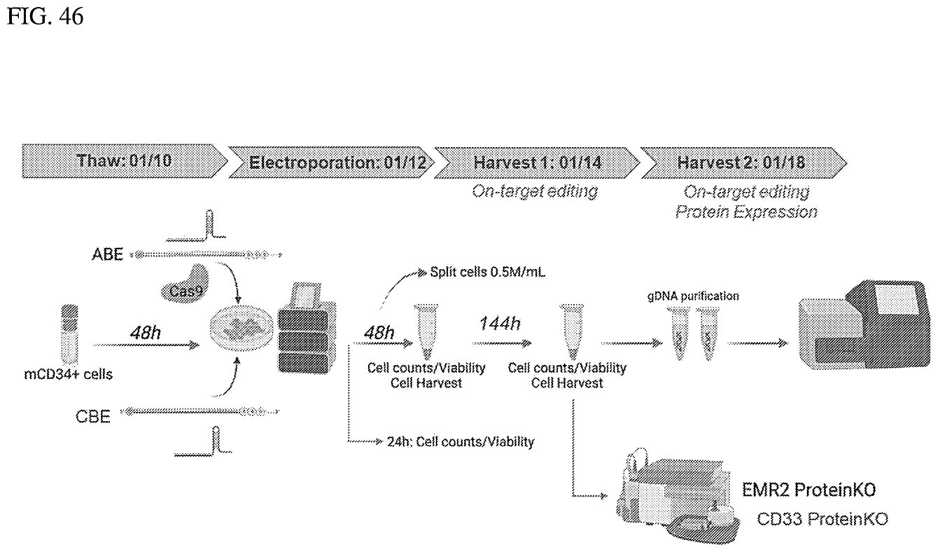

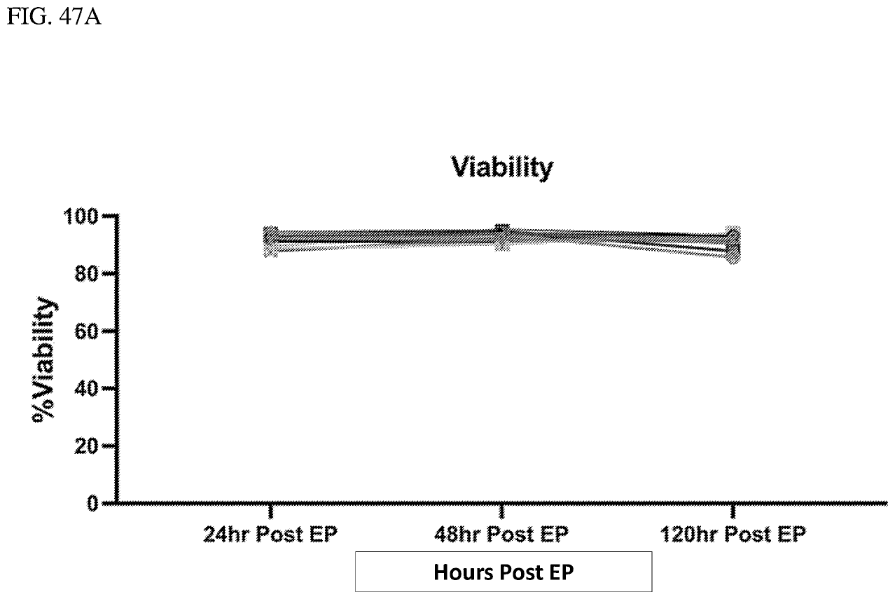

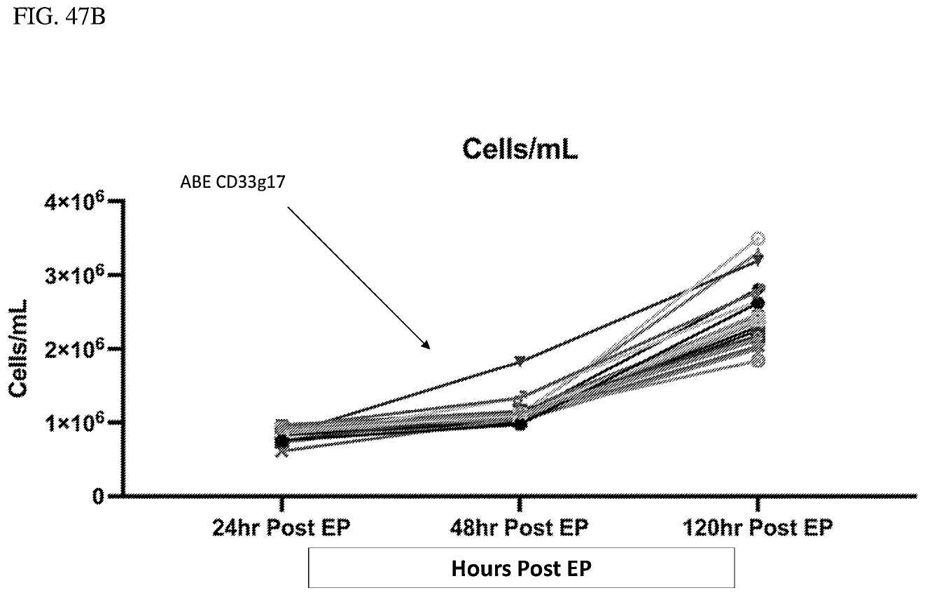

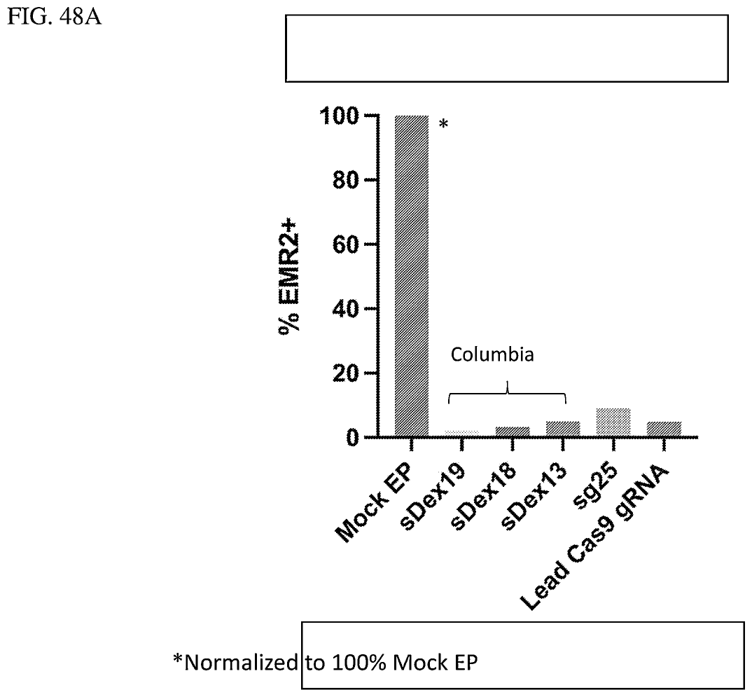

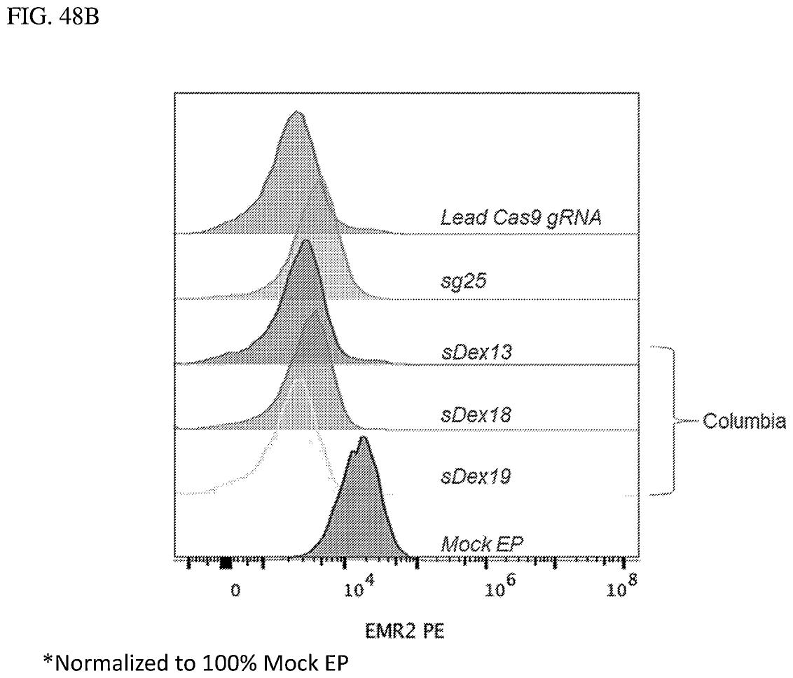

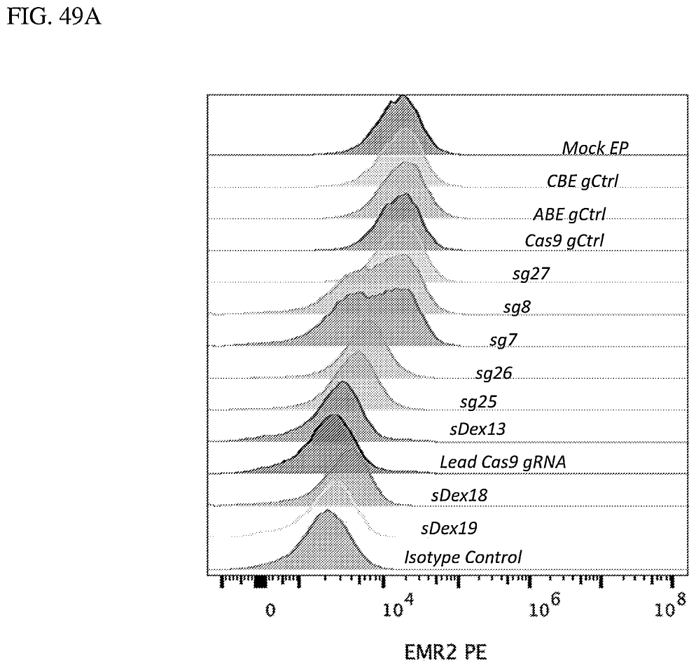

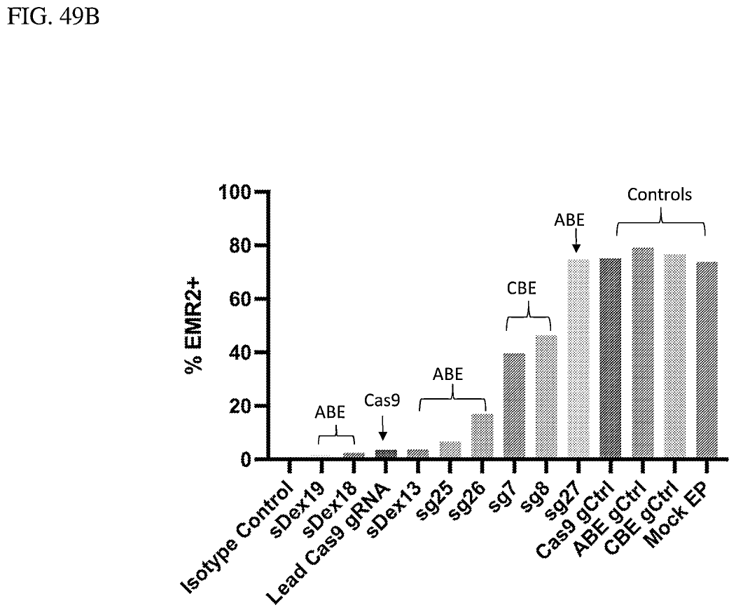

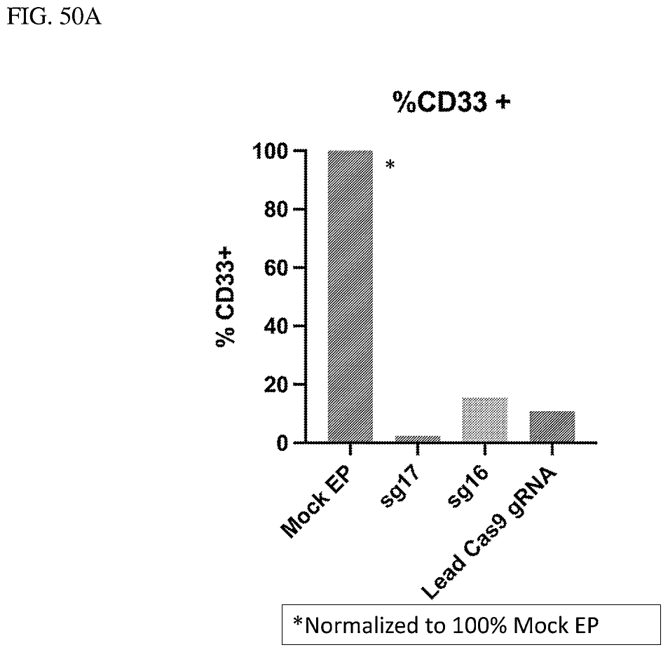

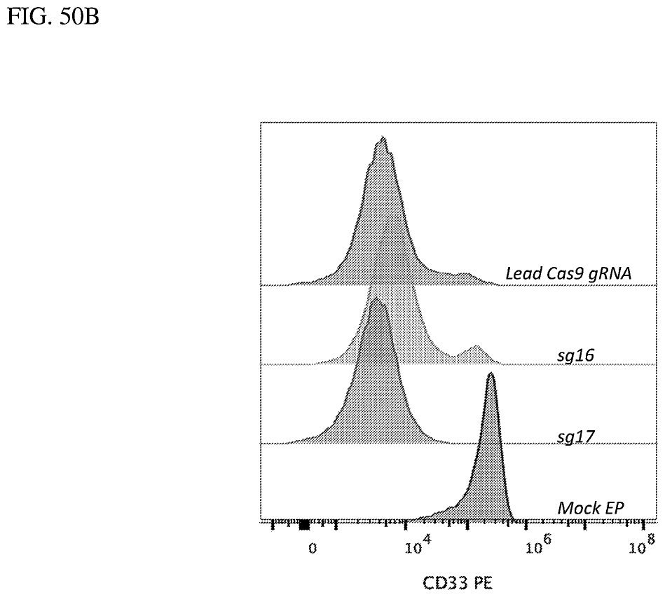

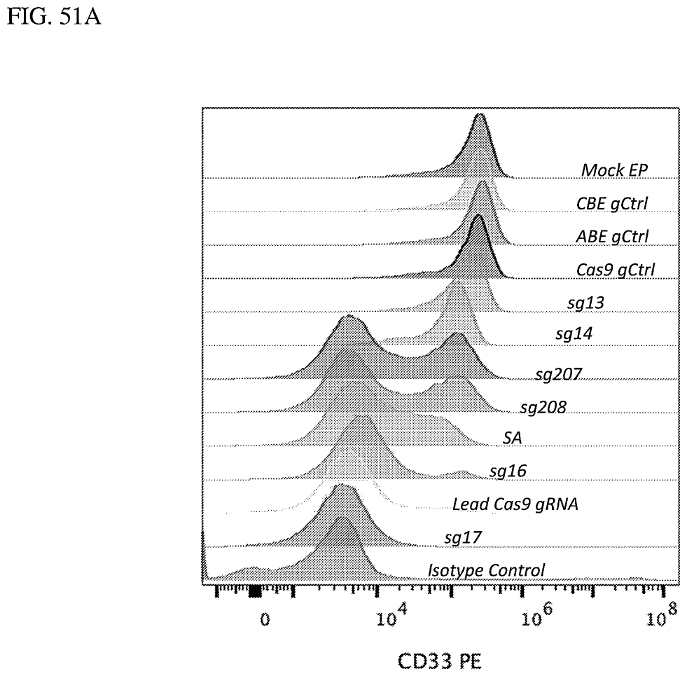

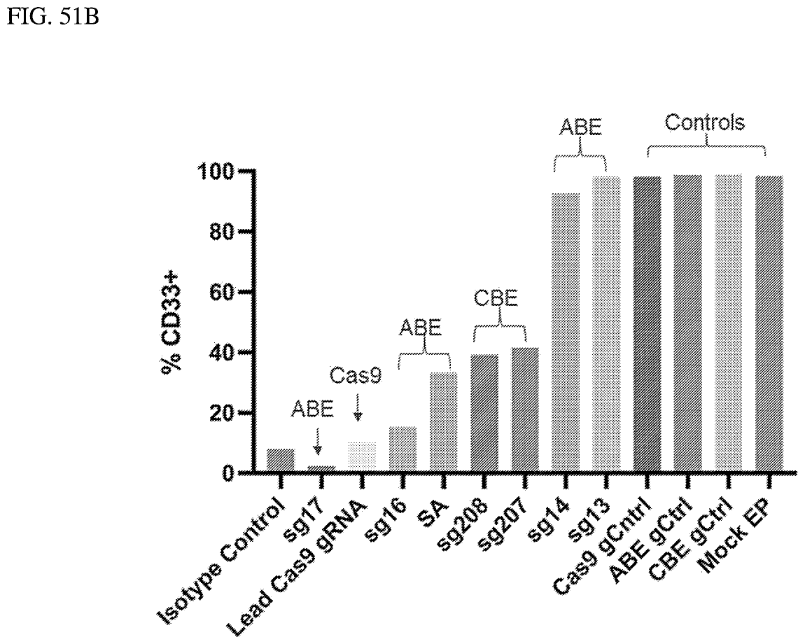

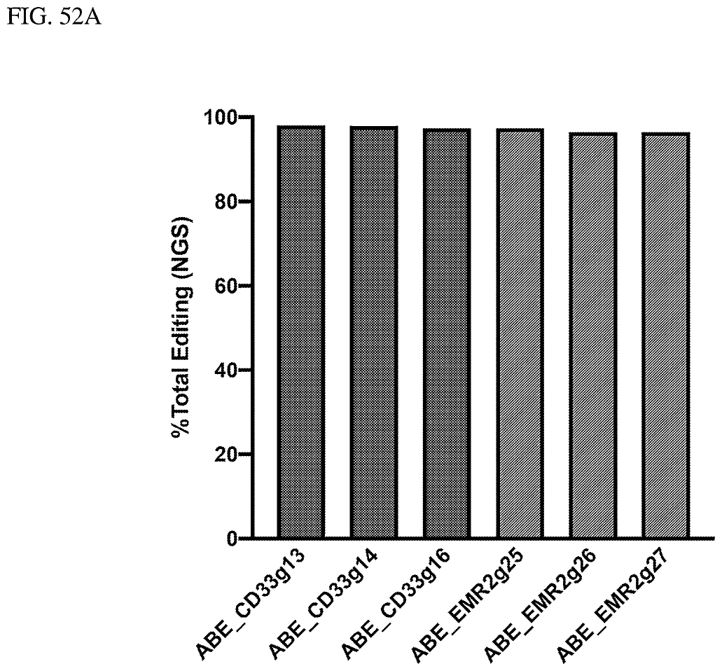

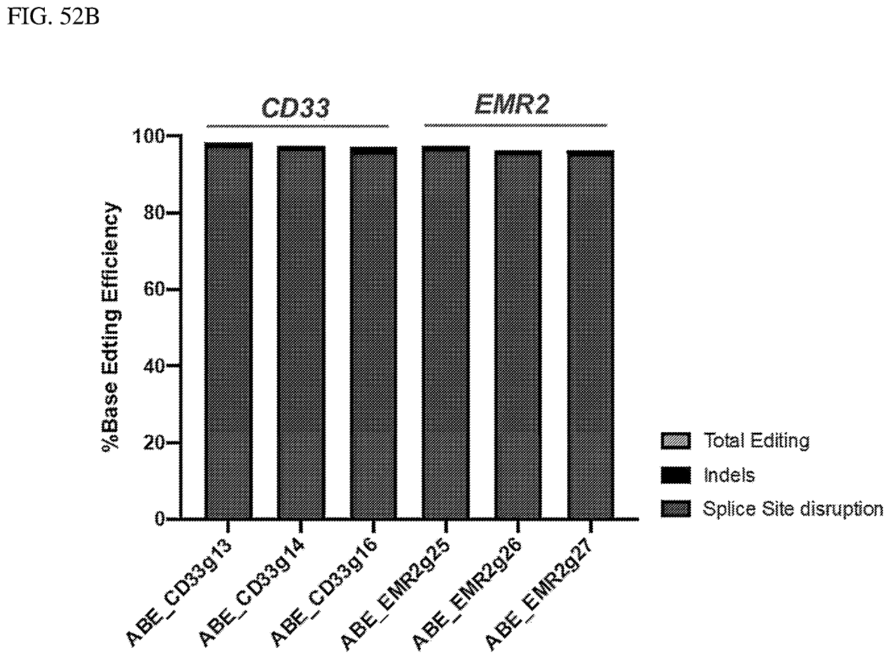

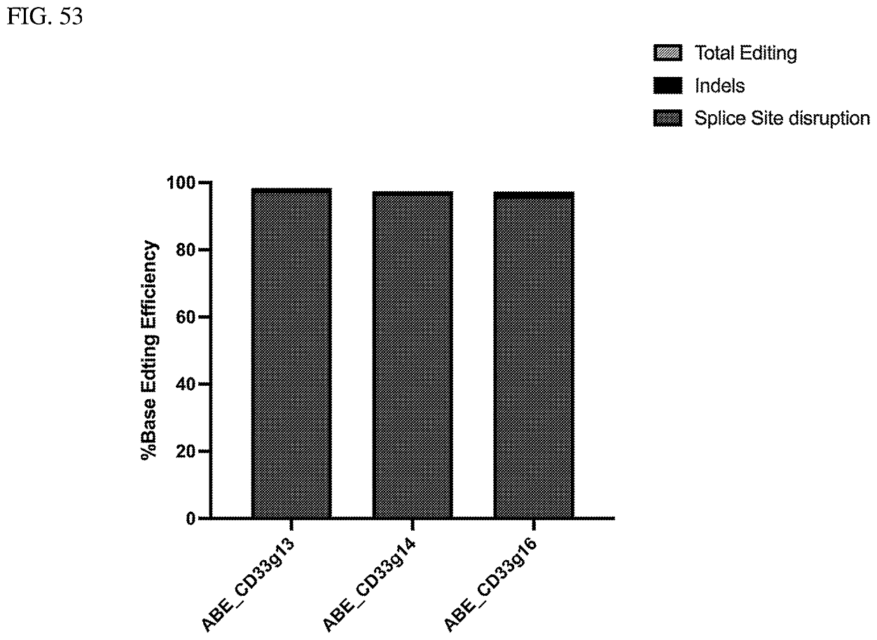

FIGS. 44 and 46 illustrates the experimental design and conditions. The CD33 and EMR2 guide screen landscape is shown in FIG. 45. FIGS. 47A-47B shows cell viability and cells counts, respectively, for CBE and ABE editing of EMR2 and CD33. FIGS. 48A-48B each show reduced surface expression of EMR2. These data demonstrate that ABE EMR2 guides show strong protein KO 6 days post EP. FIGS. 49A-49B each show that EMR2 experimental conditions resulted in varying levels of protein KO 6 days post EP. FIGS. 50A-50B each show reduced surface expression of CD33. These data demonstrate that ABE CD33 guides show strong protein KO 6 days post EP. FIGS. 51A-51B each show that CD33 experimental conditions resulted in varying levels of protein KO 6 days post EP. FIGS. 52A-52B shows total editing efficiency and base editing efficiency with ABE, respectively. The ABE guide screen in HSPCs showed high editing in various sites of CD33 and EMR2 loci and low frequencies of bystander edits. All experimental conditions showed good viability (90%) and cell expansion compared to the MockEP control. FIG. 53 shows editing efficiency of ABE CD33 gRNA.

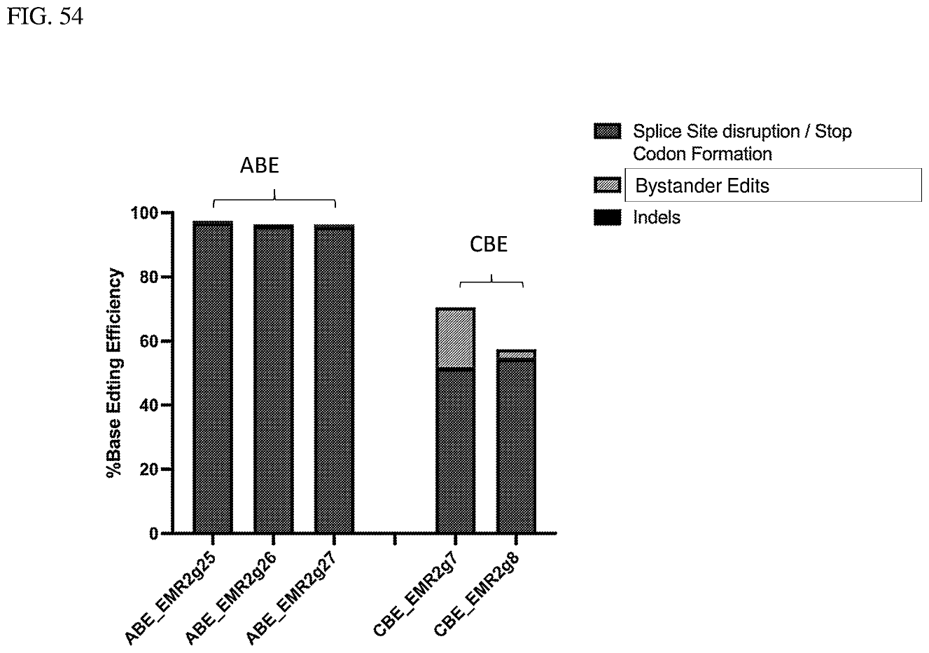

FIG. 54 shows editing efficiency of ABE and CBE EMR2 gRNAs.

### Example 12: EMR2/CD33 Multiplex ABE Base-Editing

One CD34+ donor was thawed and cultured in maintenance media (SFEM+Flt3, SCF, TPO). Two days post-thaw, ABE editing was performed. For each condition, 8e5 cells were electroporated with 9ug of ABE (N1-MPU ABE8.20m mRNA) mRNA and 4.7 uM of guide-RNA. For multiplex conditions that received an EMR2 ABE guide-RNA and CD33 ABE guide-RNA, 8e5 cells were electroporated with 9ug of ABE mRNA and 4.7 uM of both guides Additional control conditions were included. Guide-control samples received a non-targeting guide-RNA with ABE enzyme and underwent electroporation. Mock electroporation sample did not receive any enzyme or guide-RNA, but underwent electroporation. No electroporation condition did not undergo electroporation and underwent culture only. All conditions were then cultured in maintenance media (SFEM+Flt3, SCF, TPO) for five days post-electroporation. Cell counts and viability were measured using the Nexcelom Cellometer and AOPI stain (1:2 dilution) at one, two and five days post-electroporation. Cells for gDNA were harvested at two and five days post-electroporation and rhAmpSeq (NGS) was performed to measure DNA editing of ABE guides. Flow cytometry was performed at two days and five days post-electroporation to measure surface protein expression of CD33 and EMR2 in guide-edited and control samples using a cytometer. RNA pellets were also taken to be processed for transcript expression at two days and six days post-electroporation.

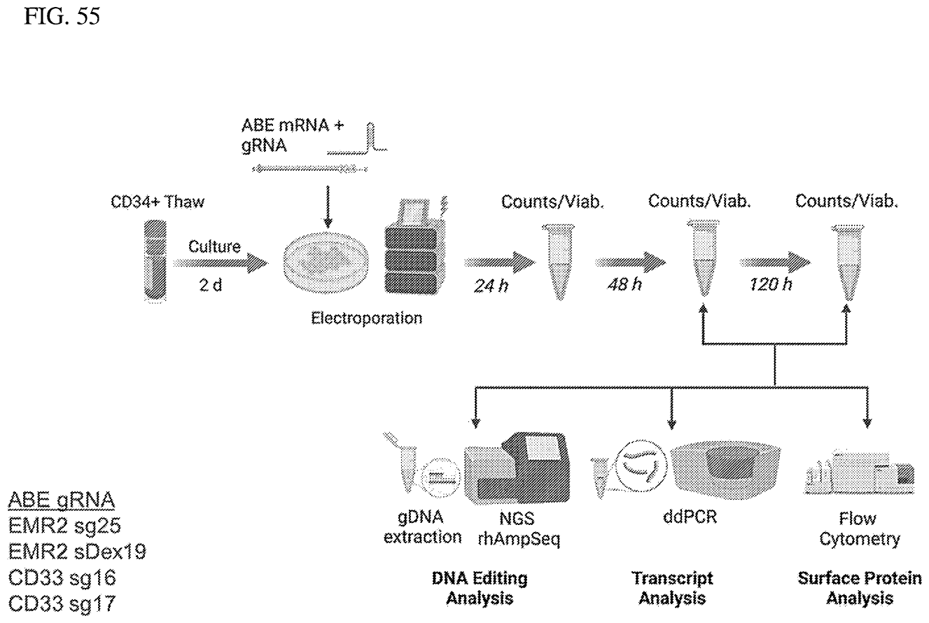

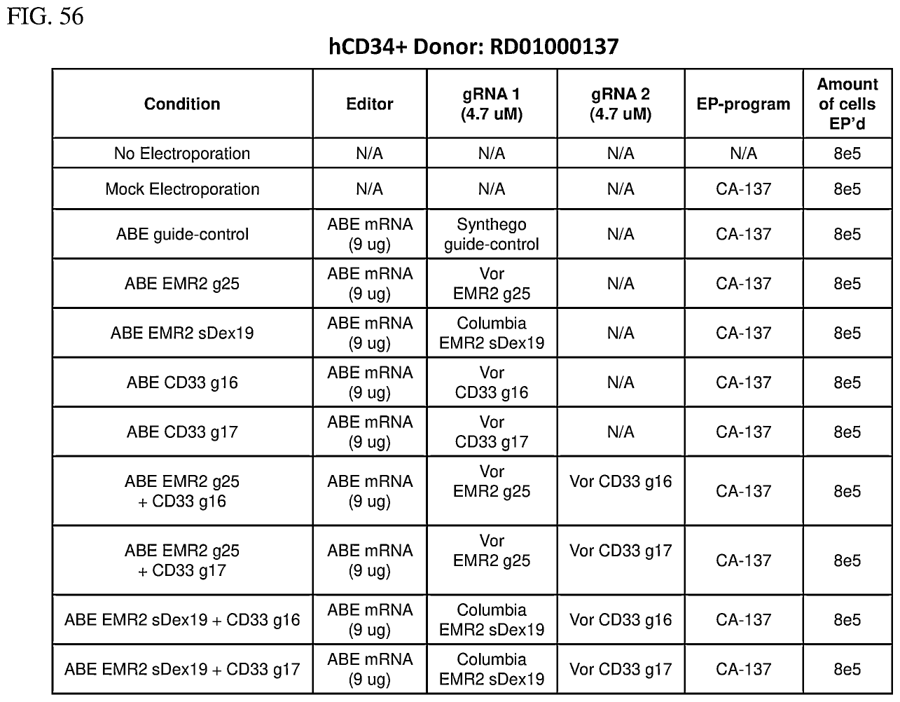

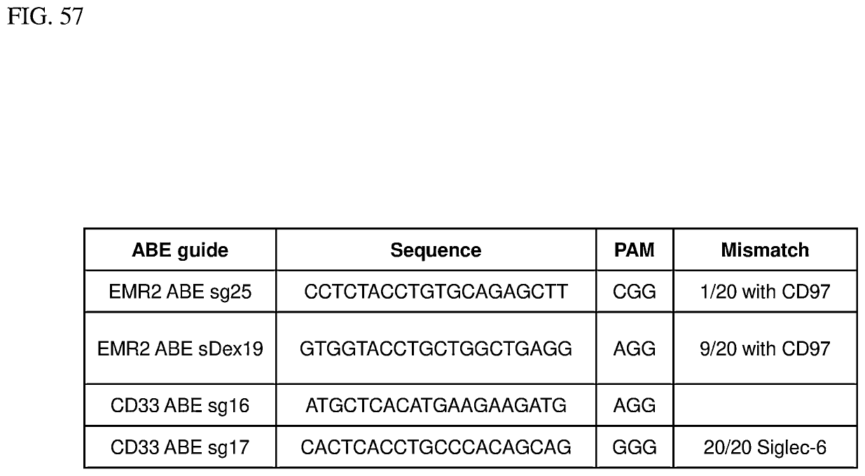

FIGS. 55-56 illustrates the experimental design and conditions. FIG. 57 shows ABE guides potential in silico off-target site.

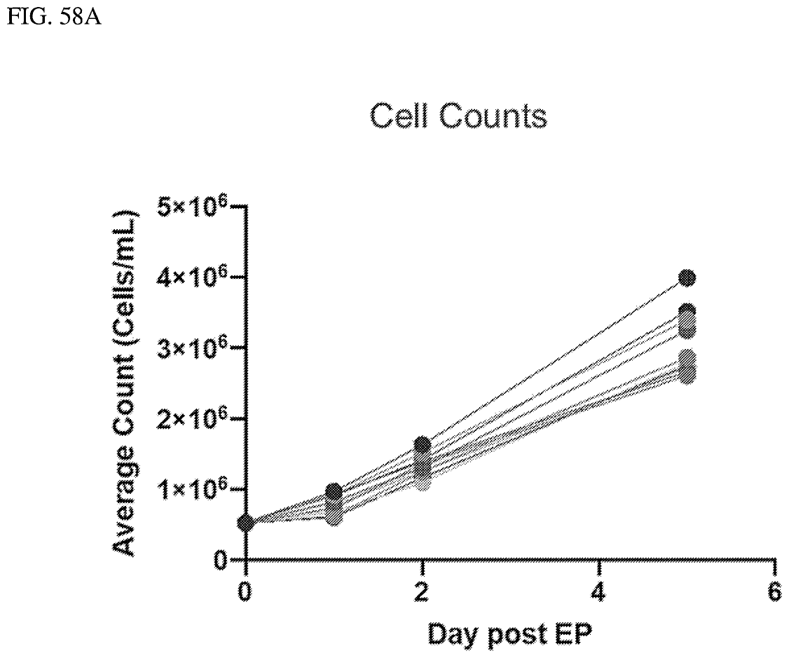

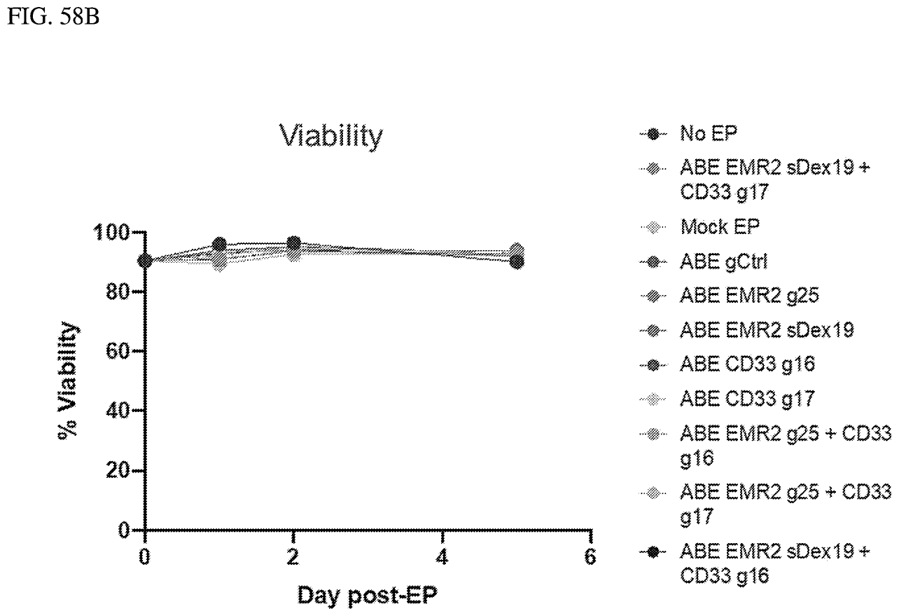

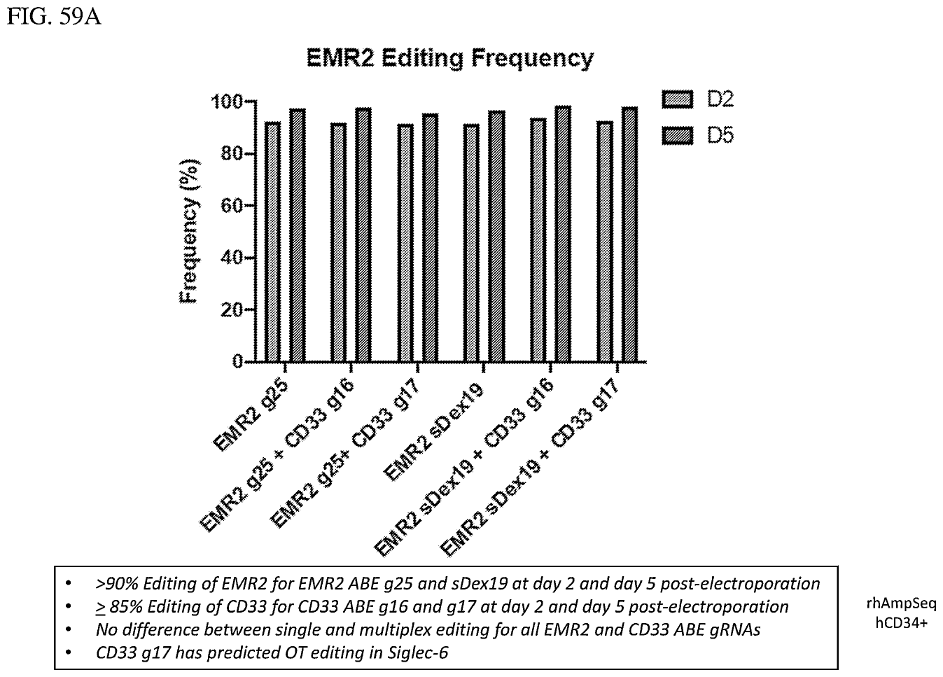

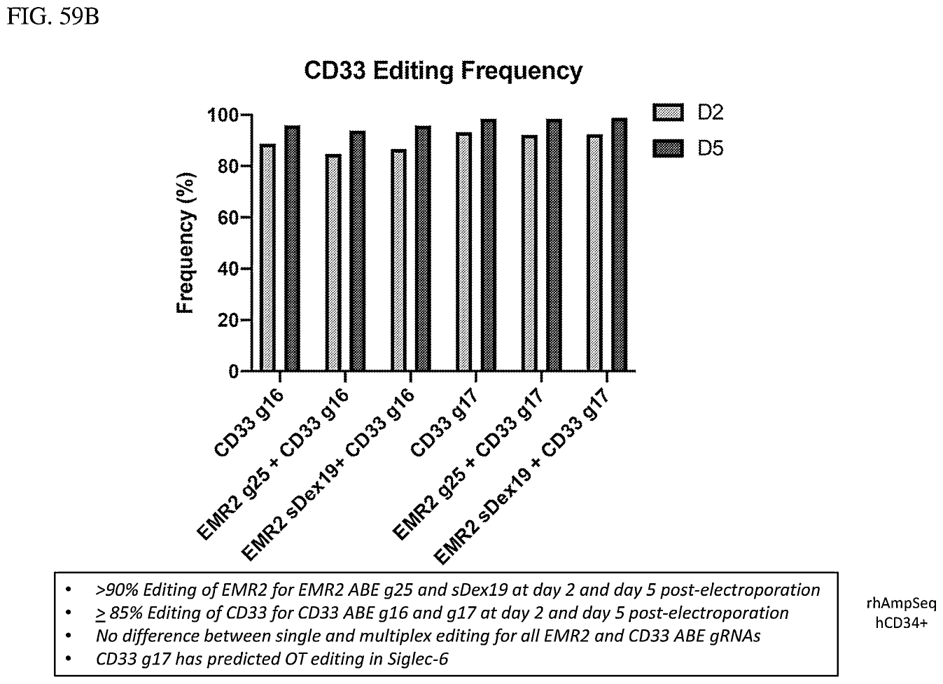

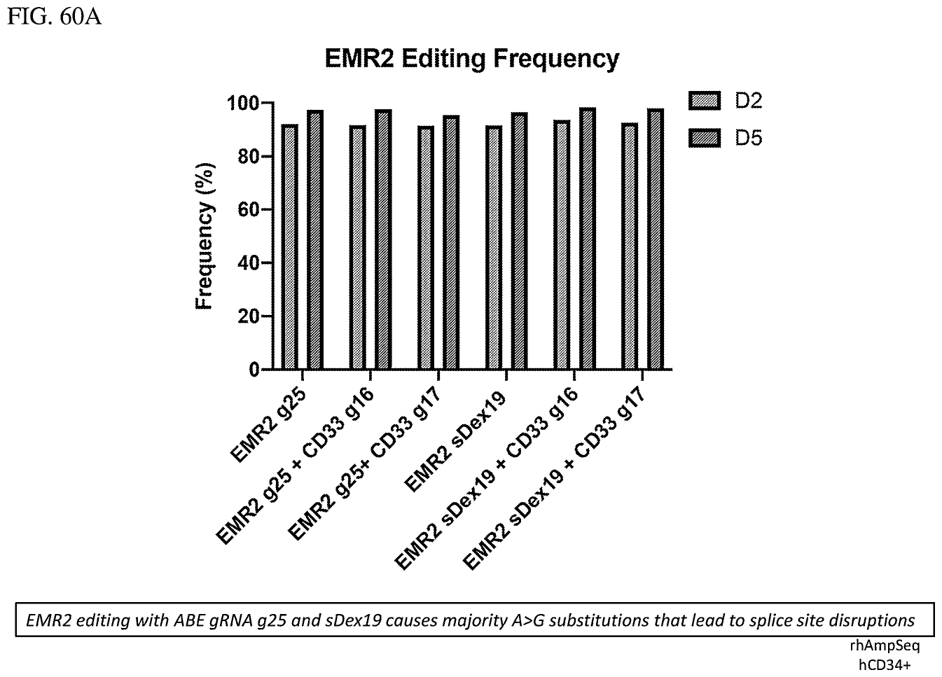

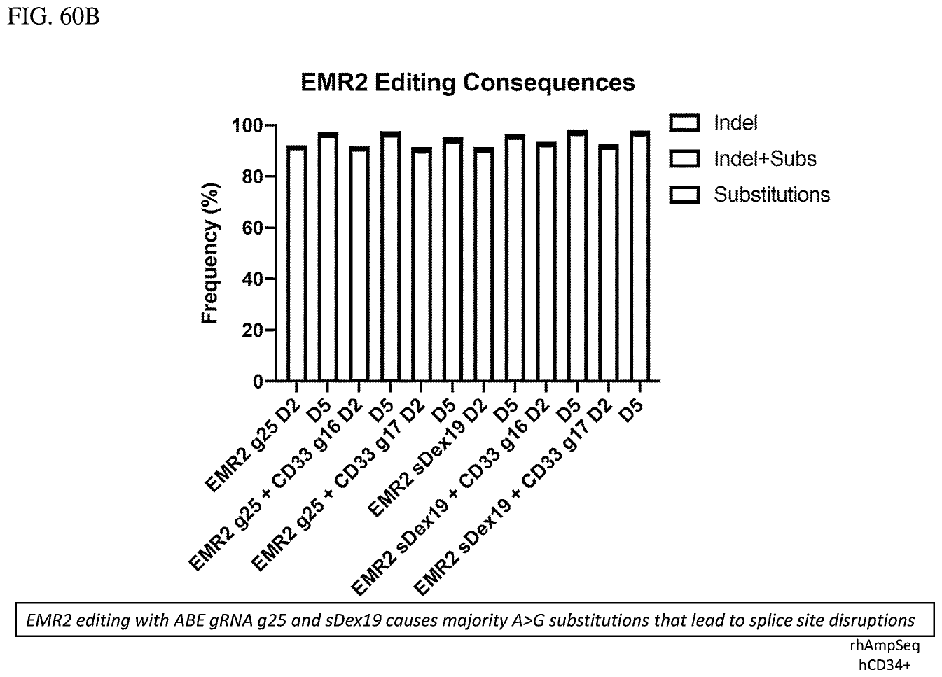

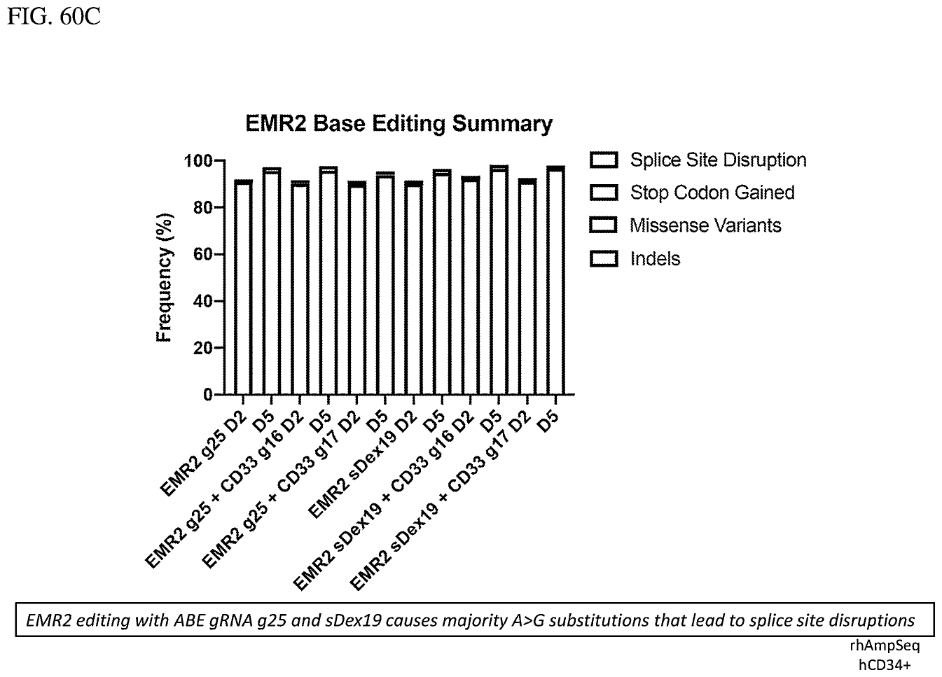

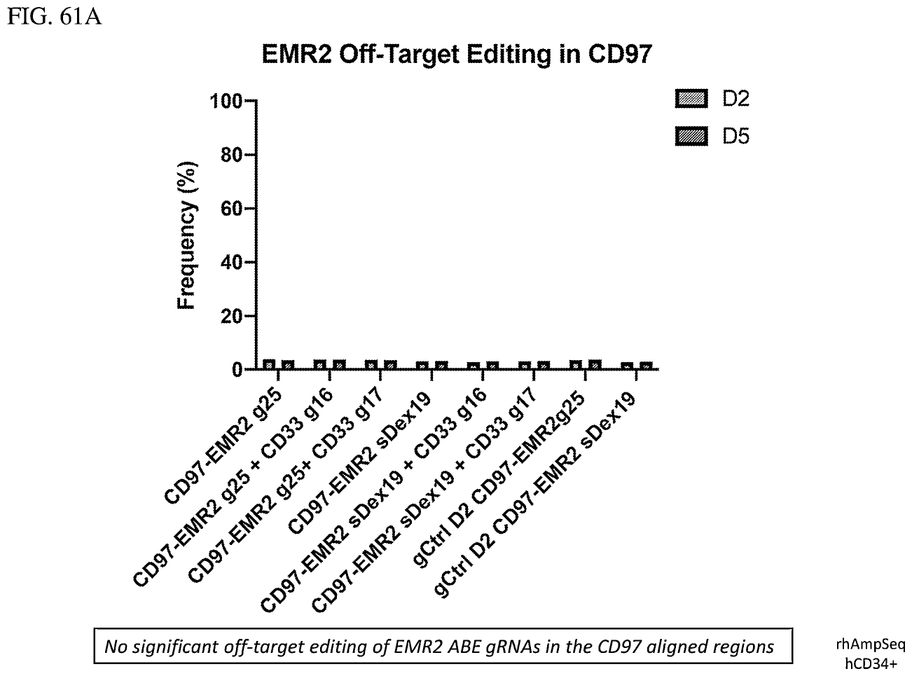

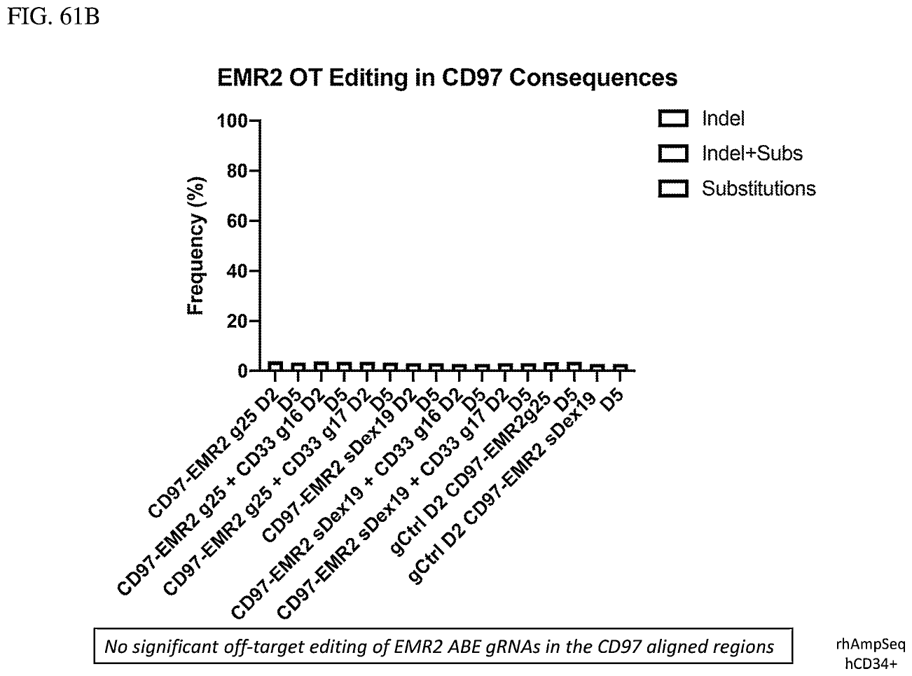

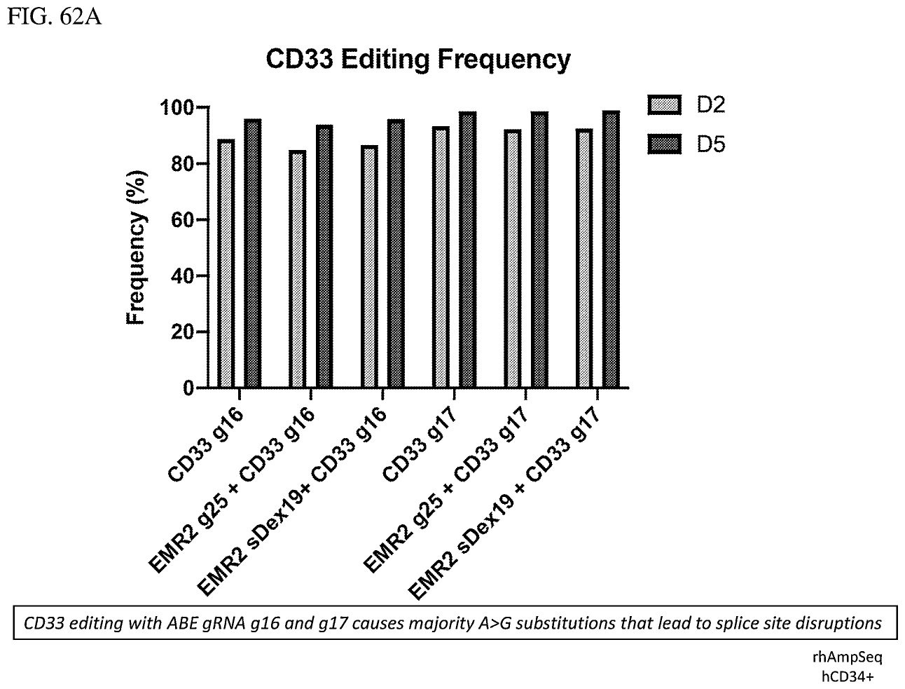

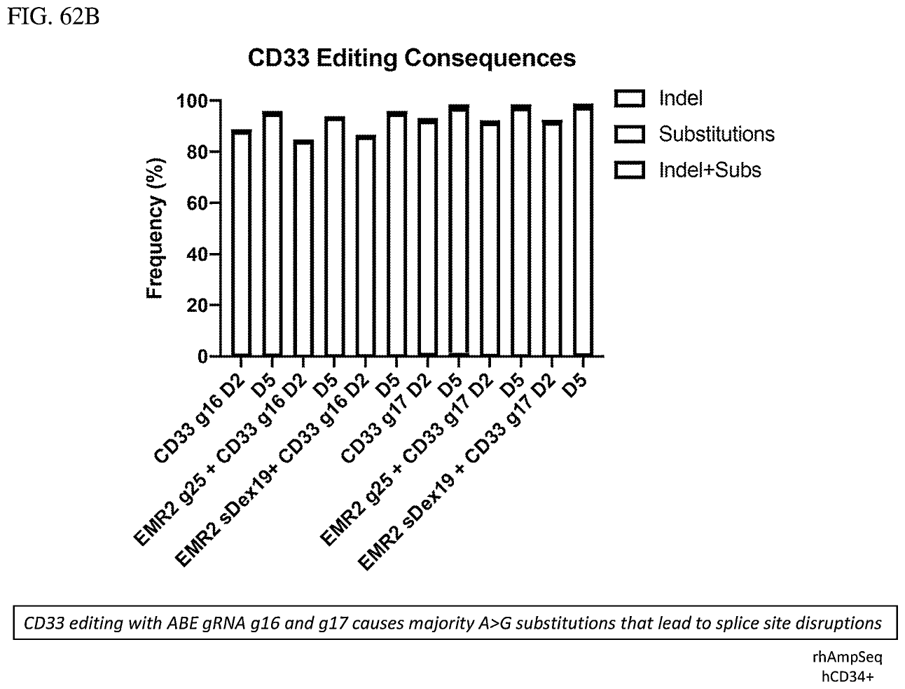

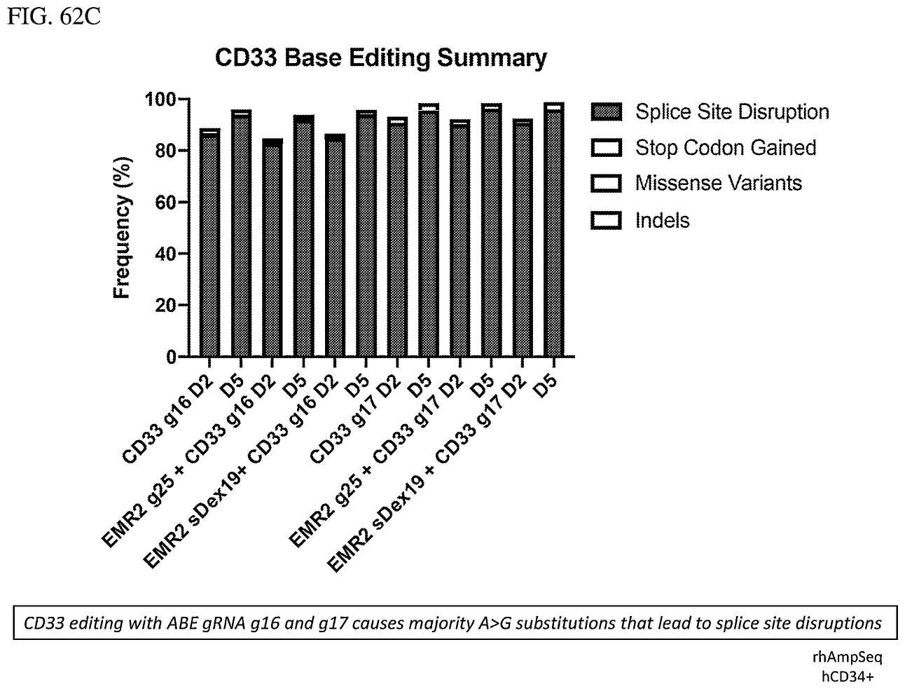

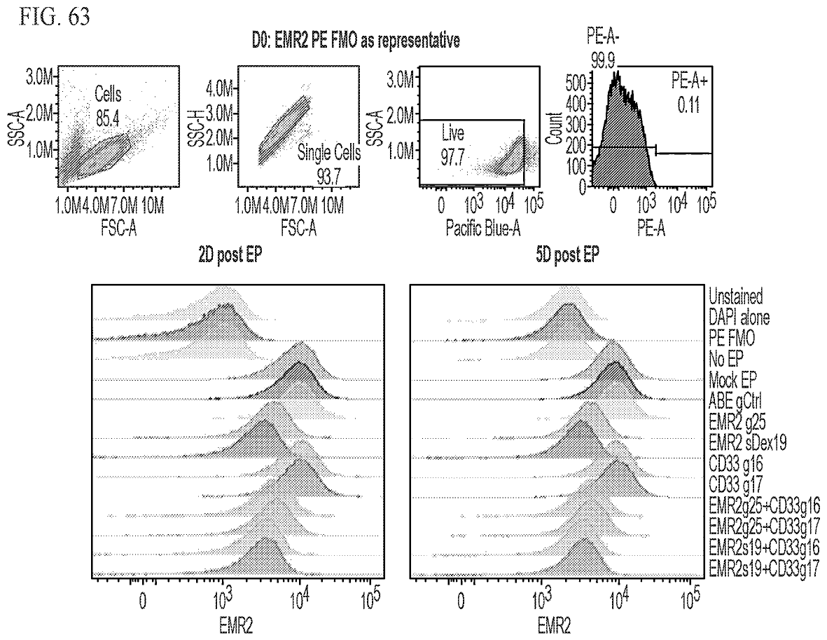

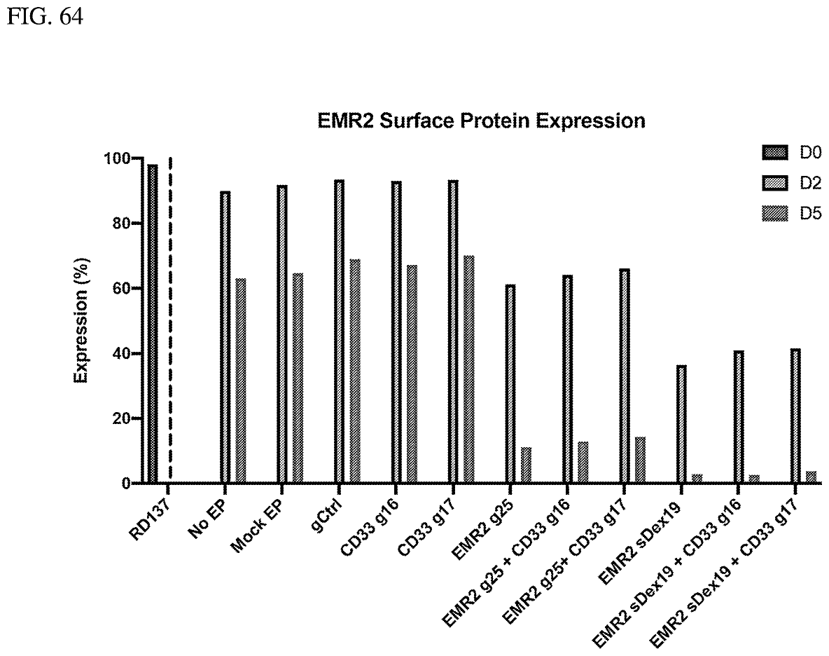

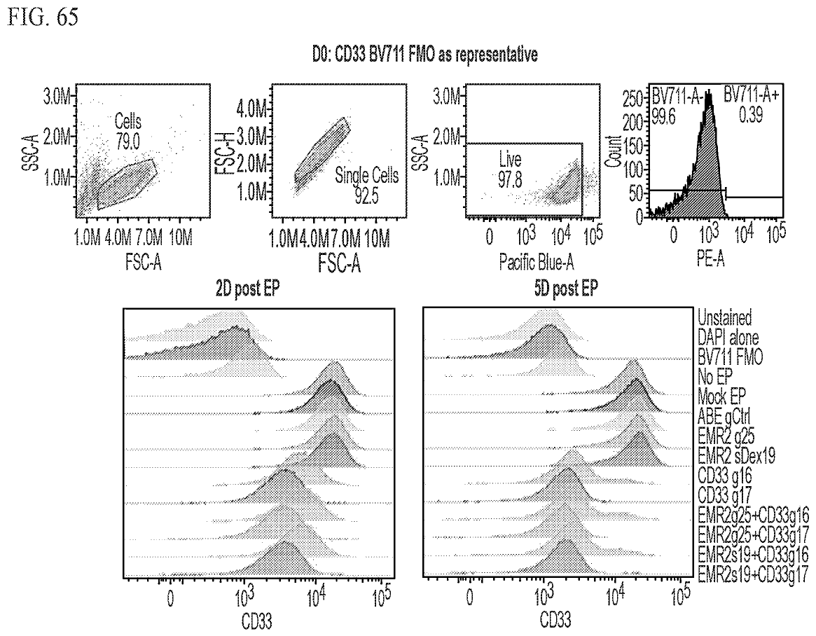

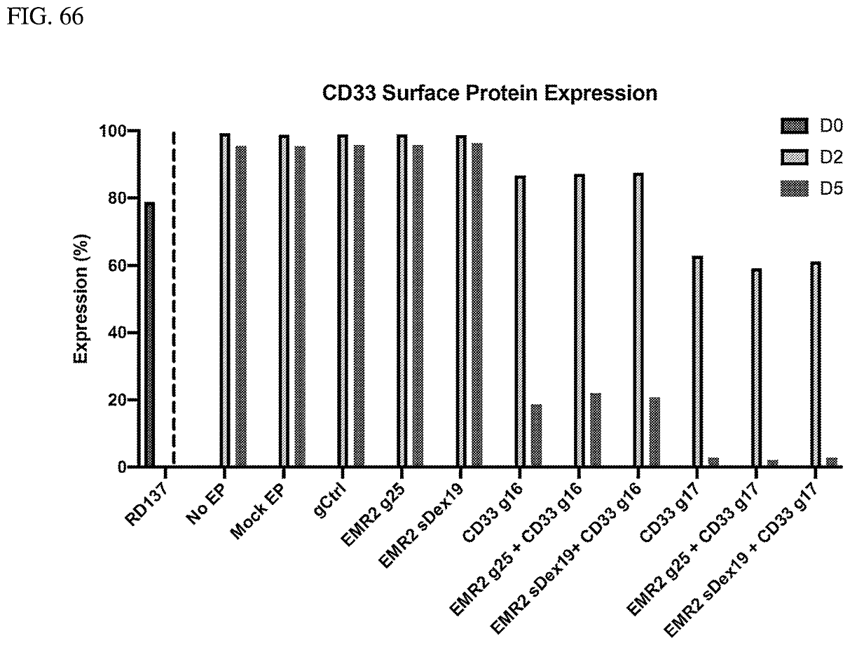

FIGS. 58A-58B show cells counts and cell viability, respectively, for ABE editing of EMR2 and CD33. FIGS. 59A-59B show ABE EMR2 and CD33 DNA editing frequency, respectively. FIGS. 60A-60C show ABE EMR2 editing frequency, editing consequences, and base editing summary, respectively. FIGS. 61A-61B show frequency of EMR2 Off-Target Editing in CD97 and consequences thereof, respectively. FIGS. 62A-62C show ABE CD33 editing frequency, editing consequences, and base editing summary, respectively. FIGS. 63-64 show EMR2 surface protein expression, while FIGS. 65-66 show CD33 surface protein expression.

### Example 13: CBE Quadraplex Base Editing of CD33, CLL1, CD123, and EMR2

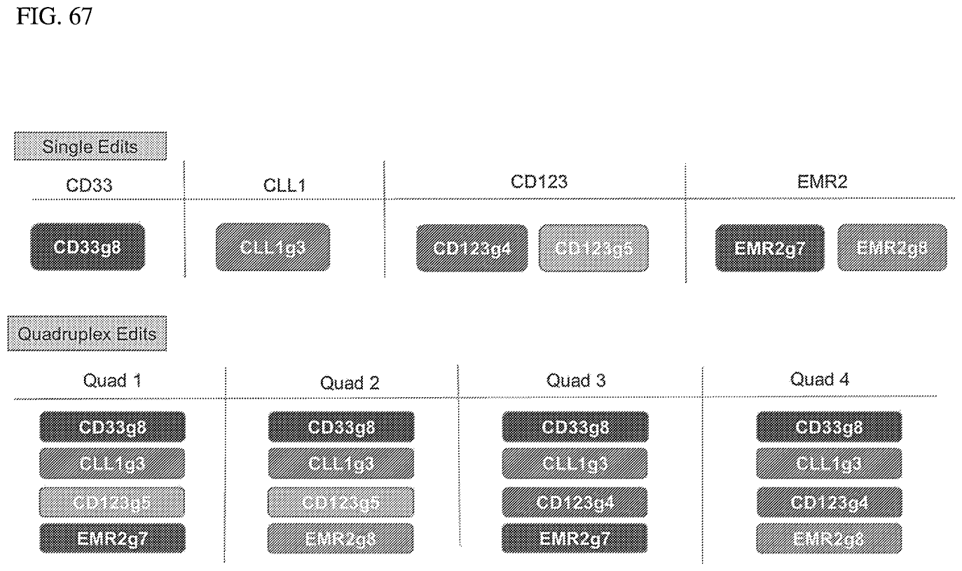

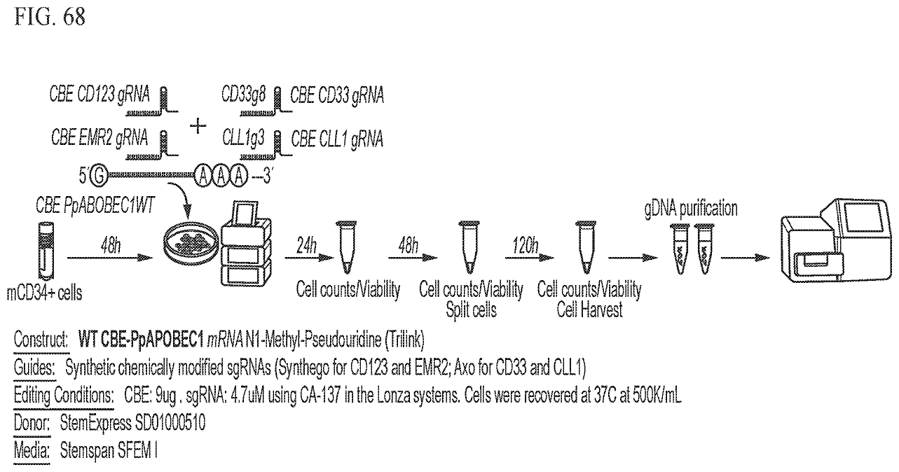

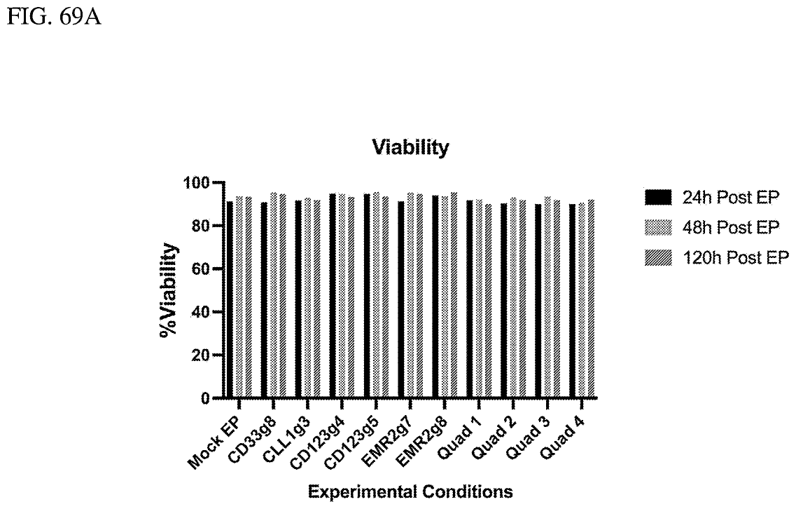

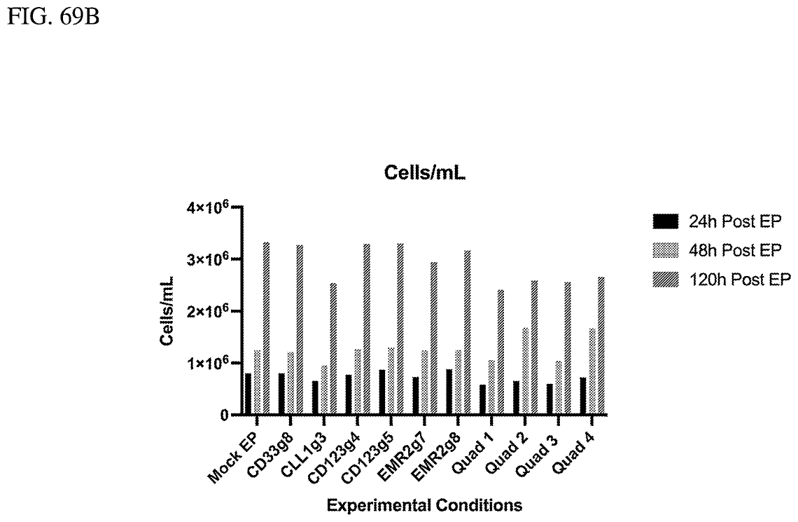

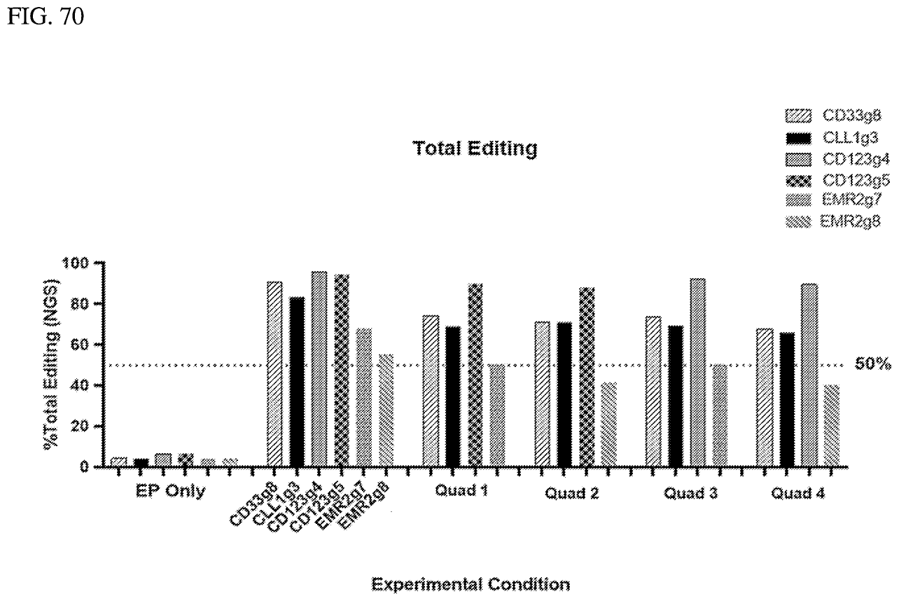

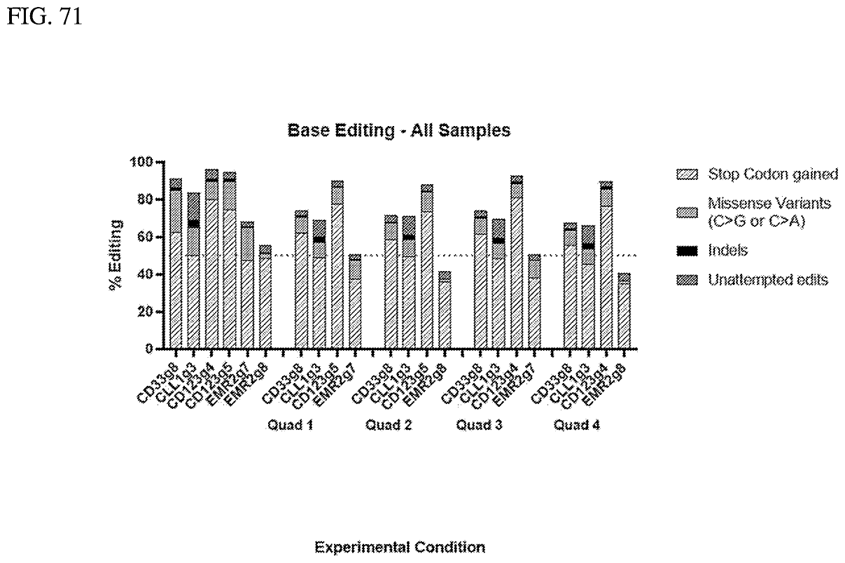

Quadraplex (i.e., simultaneous multiplex editing of four genomic targets) editing of CD34+ cells was performed using CBE and guide RNAs targeting four different genes: CD33, CLL1, CD123, and EMR2. FIG. 67 illustrates the editing conditions (i.e., guide RNA(s) used) for the single edit controls and the quadraplex edits Quad 1, Quad 2, Quad 3, Quad 4, for gene targets CD33, CLL1, CD123, and EMR2. FIG. 68 illustrates the experimental design for the quadraplex experiment. Briefly, mobilized CD34+(mCD34+) cells obtained from a single healthy donor (Donor No. SD01000510) were electroporated with a wildtype (WT) Cytosine Base Editor (CBE) mRNA construct (WT CBE-PpAPOBEC1 mRNA N1-Methyl-Pseudouridine and four guide RNAs targeting CD33, CLL1, CD123, and EMR2 genes simultaneously. Single target electroporation's with each of the CD33, CLL1, CD123, and EMR2 guide RNAs were also performed as controls. The WT CBE construct contained N1-Methyl-Pseudouridine chemical modification to improve stability of the mRNA in the cell. At day 1, mCD34+ cells were thawed for culture. After 48-hours, the CD33, CLL1, CD123, and EMR2 guide RNAs were introduced into the mCD34+ cells. After 24-hours, cell counts and cell viability were assessed. After 48-hours, cell counts and cell viability was again assessed, and the cells were harvested. gDNA was purified and the editing readout obtained via NGS. In each case 9 micrograms of CBE was complexed with 4.7 uM sgRNA. Pellets of the cells 120 hours post electroporation (EP) were prepared for sequencing the editing readout obtained via next-generation sequencing (NGS). Computational analysis of the sequencing results was used to determine knockout (KO) of the CD33, CLL1, CD123, and EMR2 protein expression. FIGS. 69A-69B shows cell viability and cell expansion (cells/mL) 24 h, 48h and 120h post electroporation (EP), demonstrating that Quadruplex Editing did not impact cell health with no significant effect on cell viability or cell expansion. FIG. 70 shows the levels of total editing as measured via NGS for the single edit controls and the quadraplex edits Quad 1, Quad 2, Quad 3, Quad 4, for gene targets CD33, CLL1, CD123, and EMR2, or EP only controls) Overall, on-target editing was achieved for each target in each of the quadraplex conditions. The on-target editing of the single guides were comparable to those in each of the quadraplex conditions, just slightly reduced. FIG. 71 shows the percentage editing for various edits: stop codon gained, missense variants (C>G or C>A), indels or unattempted edits for the single edit controls and the quadraplex edits Quad 1, Quad 2, Quad 3, Quad 4, for gene targets CD33, CLL1, CD123, and EMR2. The base editing distribution (among stop codon gain, missense variants, indels and unattempted edits) were comparable as a single target as they were in the corresponding quadraplex conditions. These data demonstrate the feasibility and success achievable with simultaneous quadraplex base editing. These experiments achieved approximately 50% or greater CD33/CLL1/CD123, and EMR2 quad edited cells, except for CBE EMR2g8 in quadraplex conditions (˜40%). Notably, there was no impact on cell viability or cell expansion.

### Example 14: ABE CD33/CD123/EMR2 Triple KO

One CD34+ donor was thawed and cultured in maintenance media (SFEM+Flt3, SCF, TPO). Two days post-thaw, ABE editing was performed. For each condition, 1e6 cells were electroporated with 9ug of ABE (N1-MPU ABE8.20m mRNA) mRNA and 4.7 uM of guide-RNA. For triplex conditions that received an EMR2 ABE guide-RNA and CD33 ABE guide-RNA and CD123 ABE guide-RNA, 1e6 cells were electroporated with 9 μg of ABE mRNA and 4.7 uM of all three guides Additional control conditions were included. Guide-control samples received a non-targeting guide-RNA with ABE enzyme and underwent electroporation. Mock electroporation sample did not receive any enzyme or guide-RNA, but underwent electroporation. No electroporation condition did not undergo electroporation and underwent culture only. All conditions were then cultured in maintenance media (SFEM+Flt3, SCF, TPO) for five days post-electroporation. Cell counts and viability were measured using a Cellometer and AOPI stain (1:2 dilution) at one, two and five days post-electroporation. Cells for gDNA were harvested at two and five days post-electroporation and rhAmpSeq (NGS) was performed to measure DNA editing of ABE guides. Flow cytometry was performed at two days and five days post-electroporation to measure surface protein expression of CD33, CD123 and EMR2 in guide-edited and control samples and to assess triple knock-out efficiency using a cytometer. RNA pellets were also taken to be processed for transcript expression at two days and six days post-electroporation.

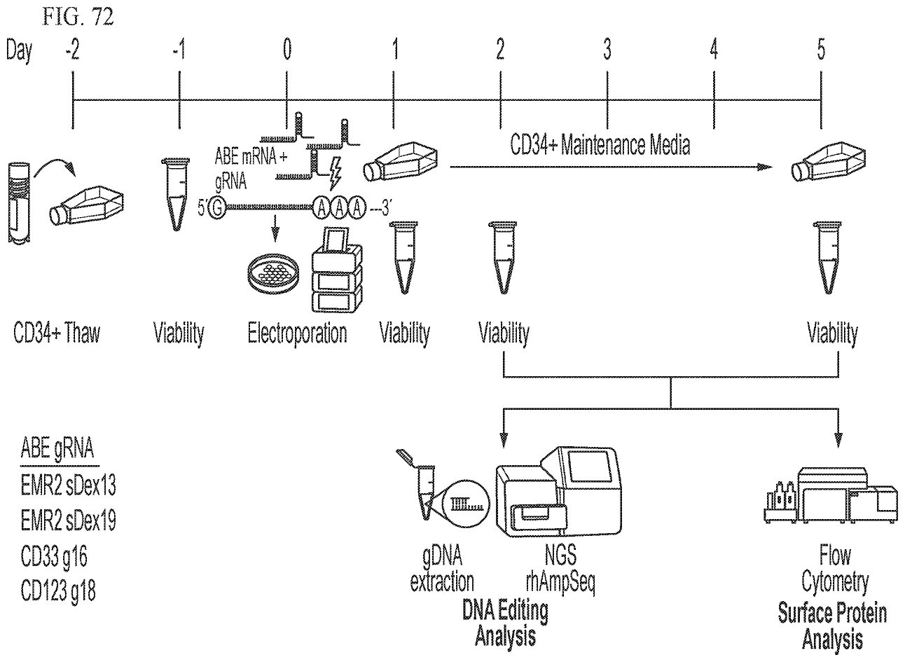

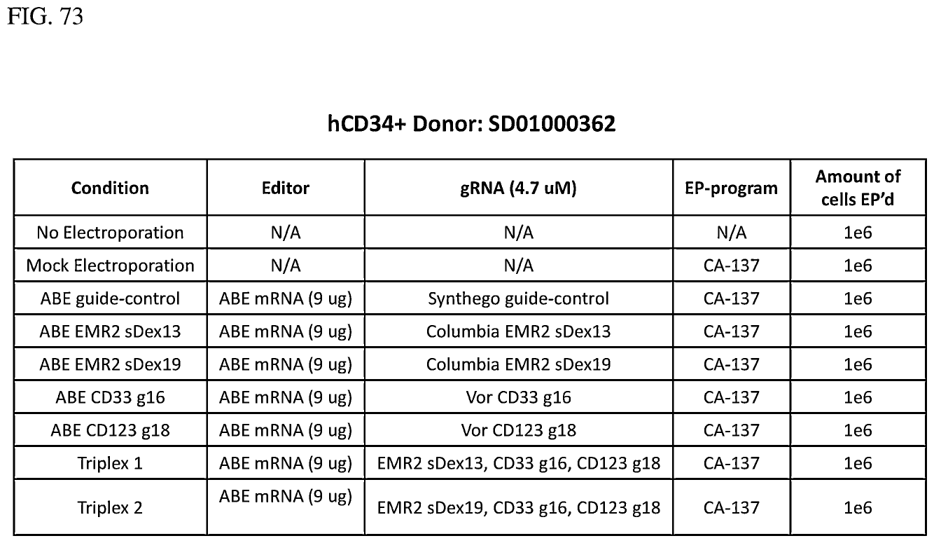

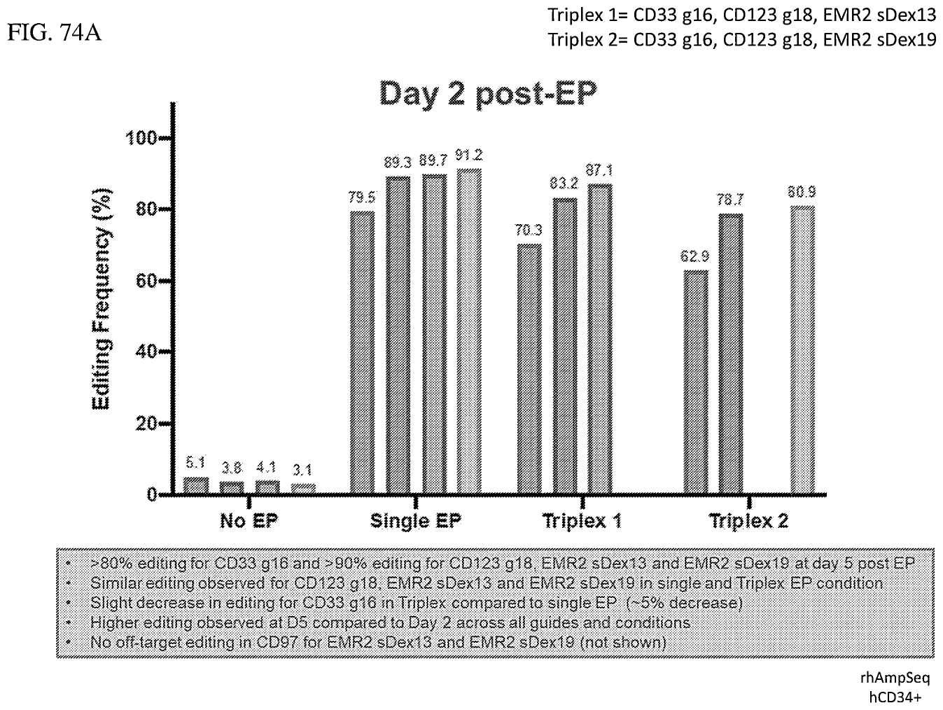

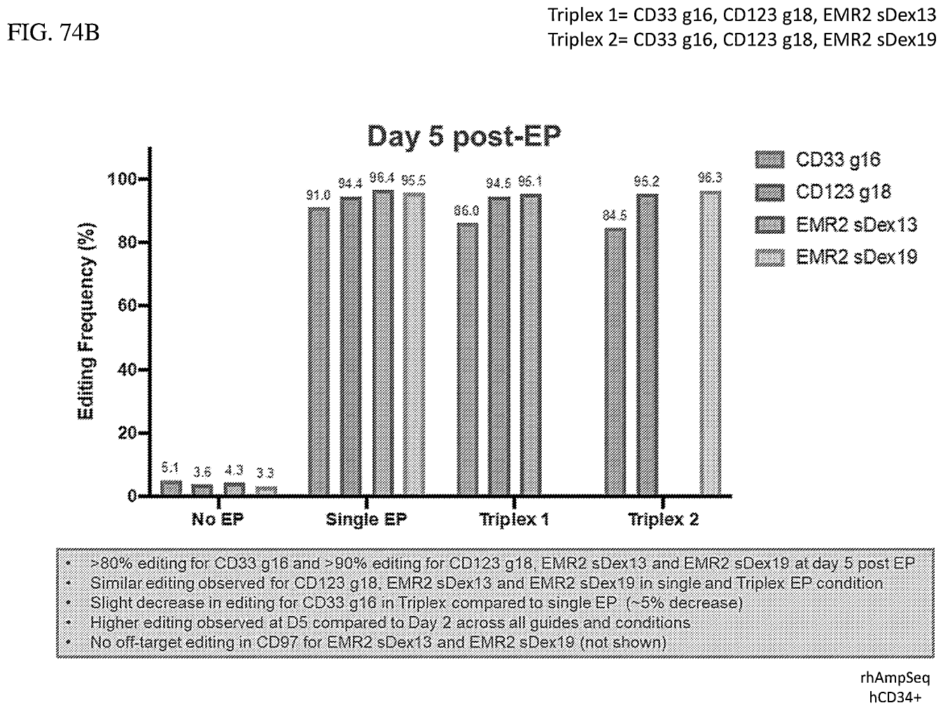

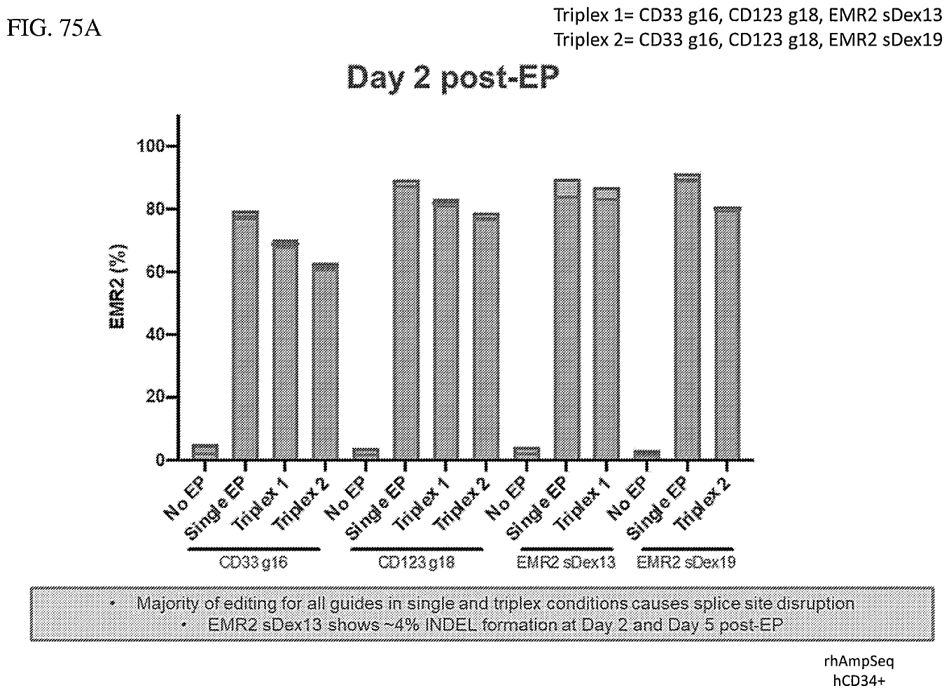

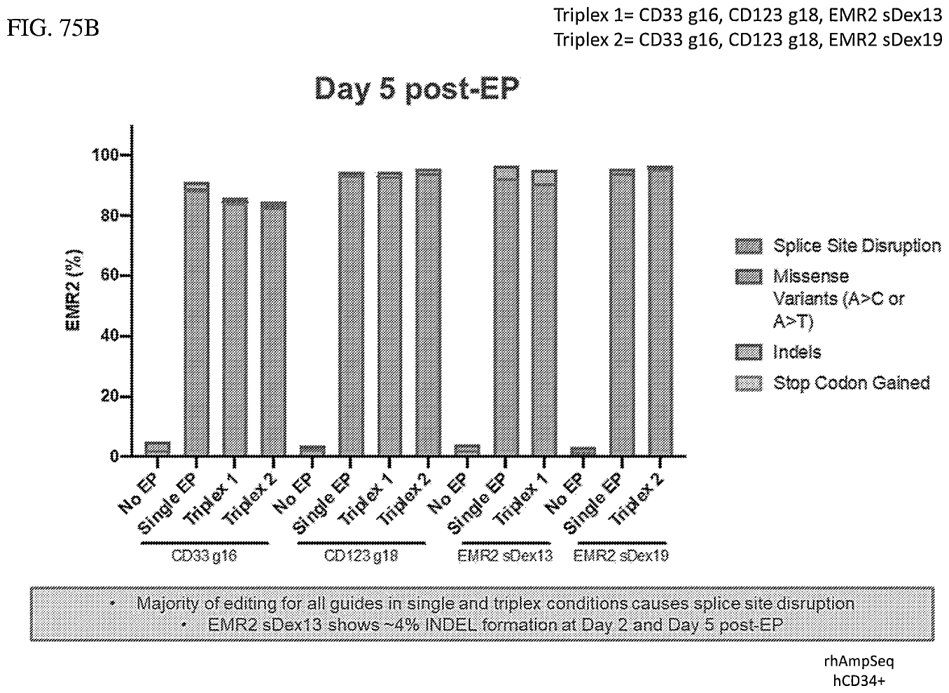

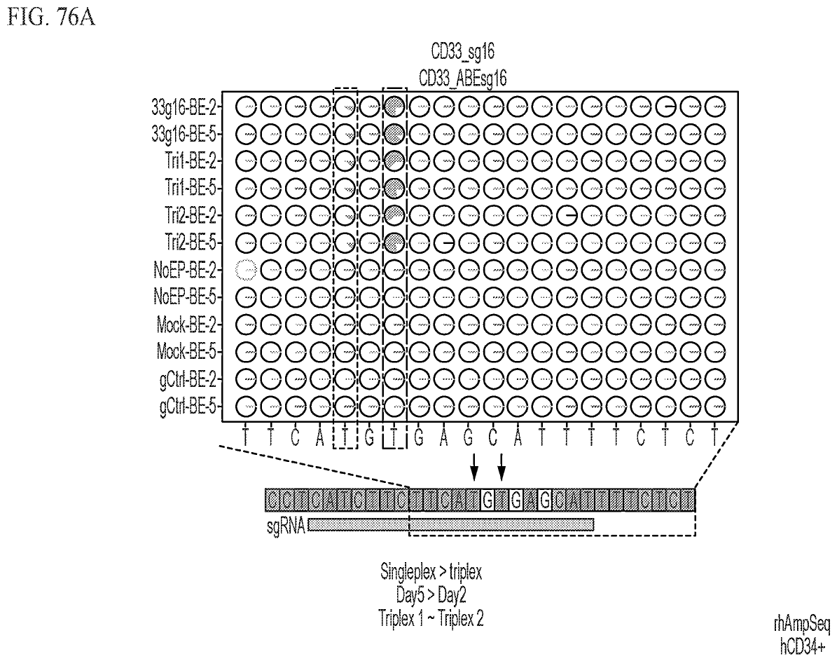

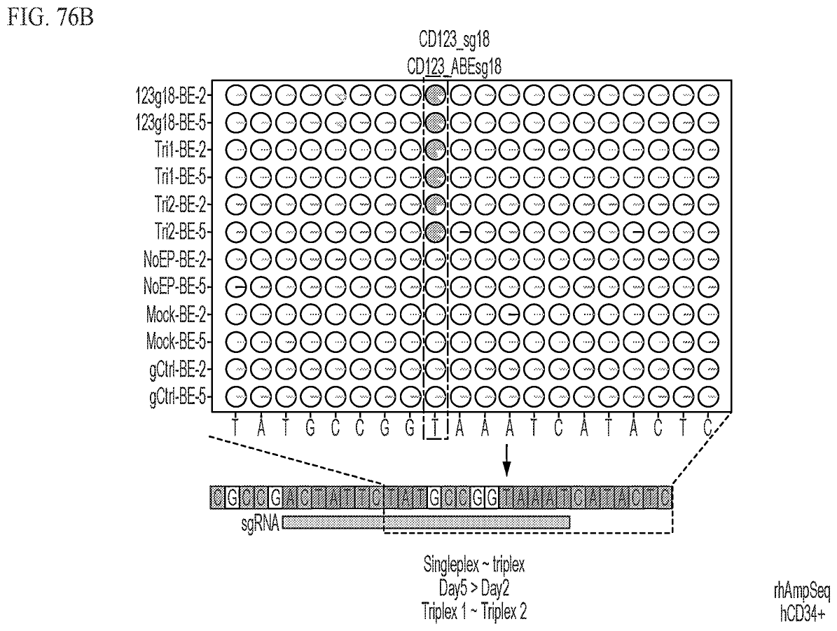

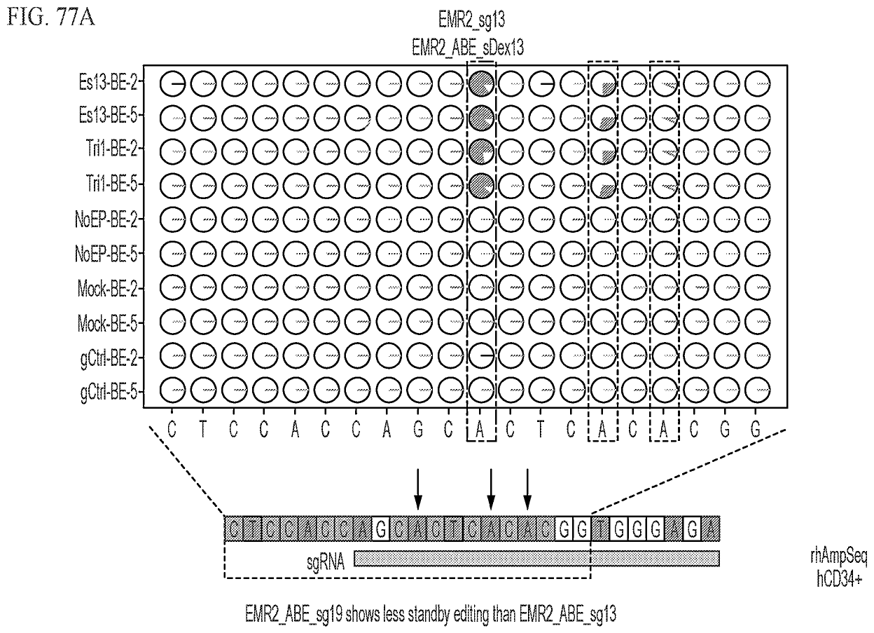

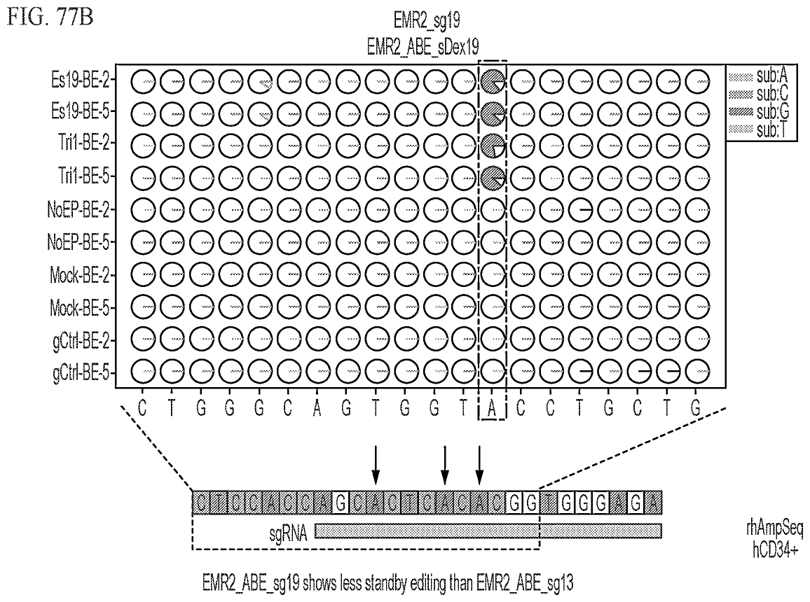

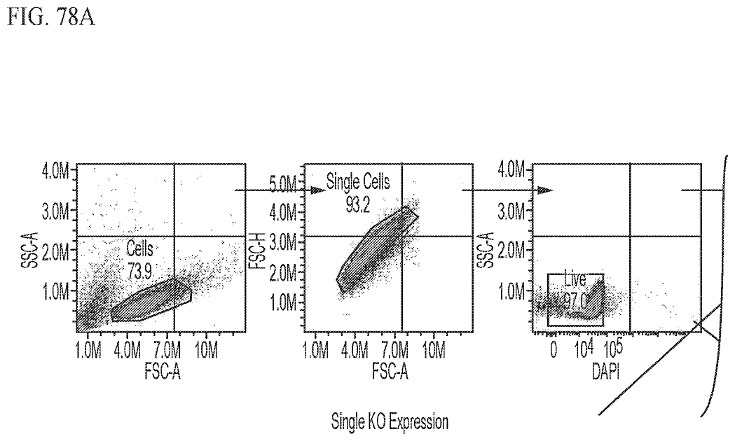

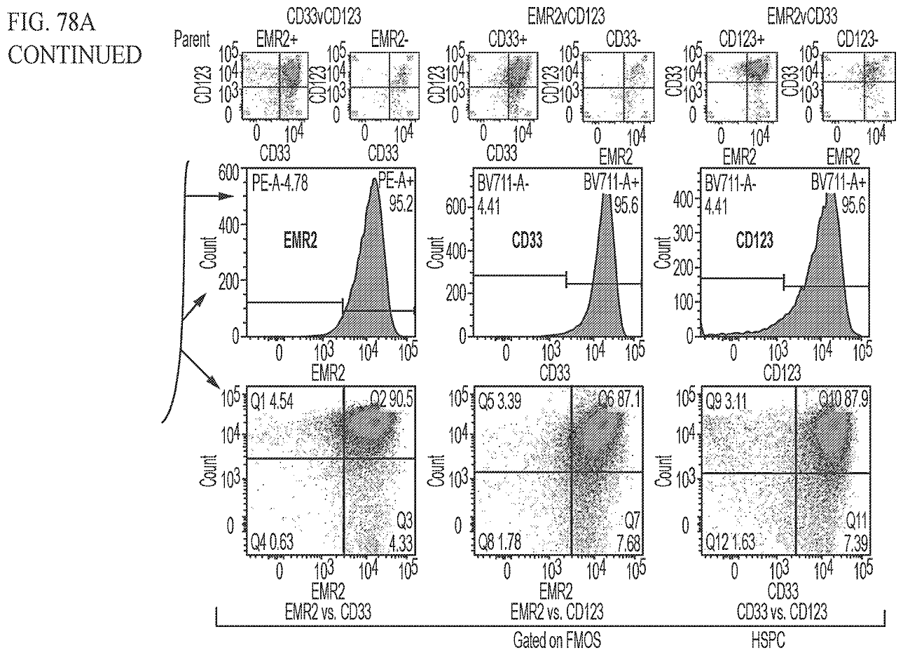

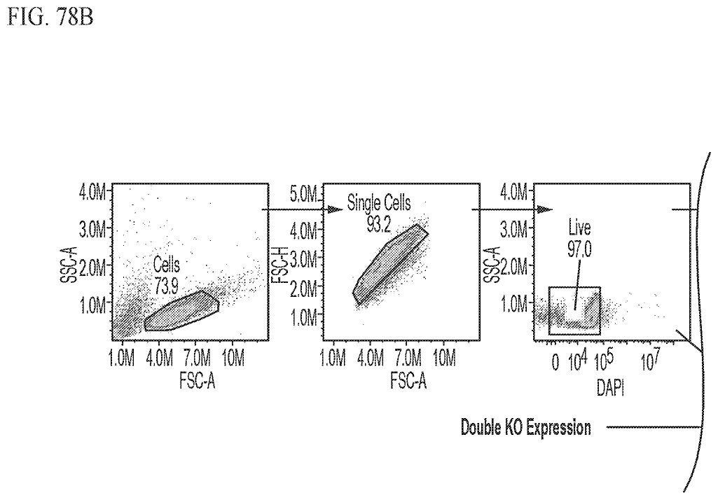

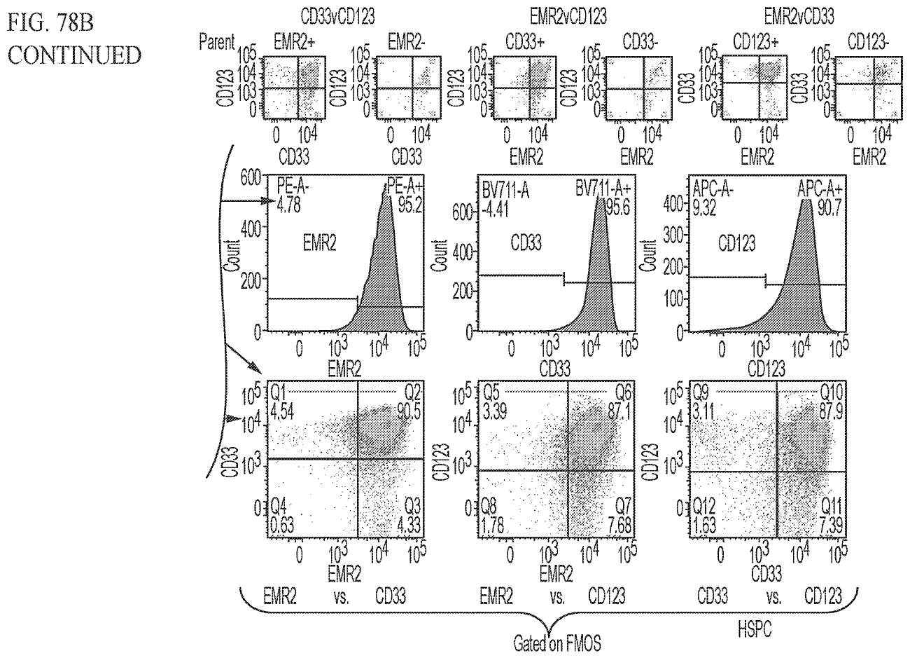

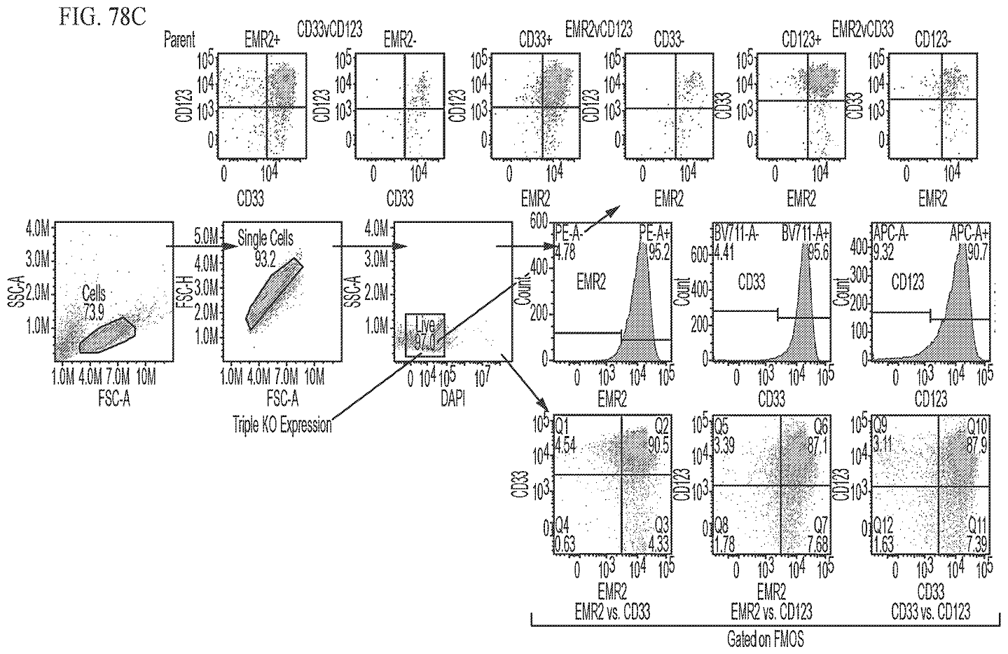

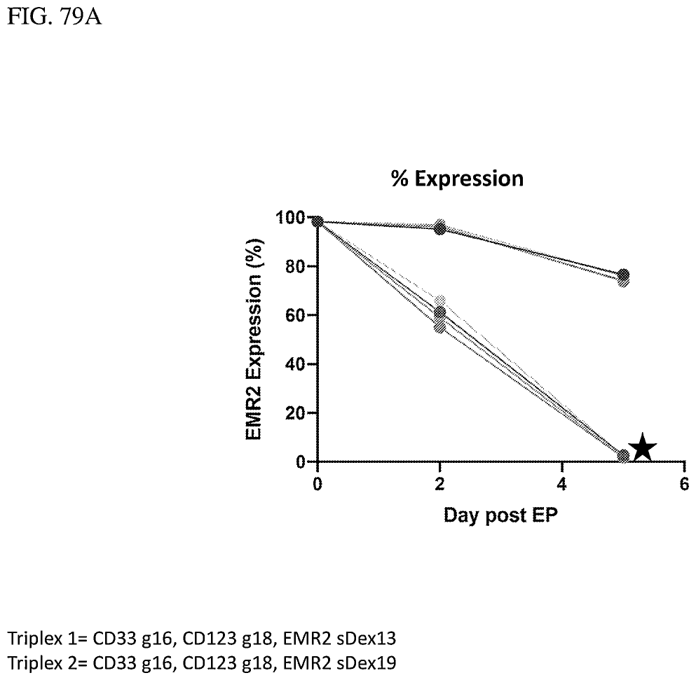

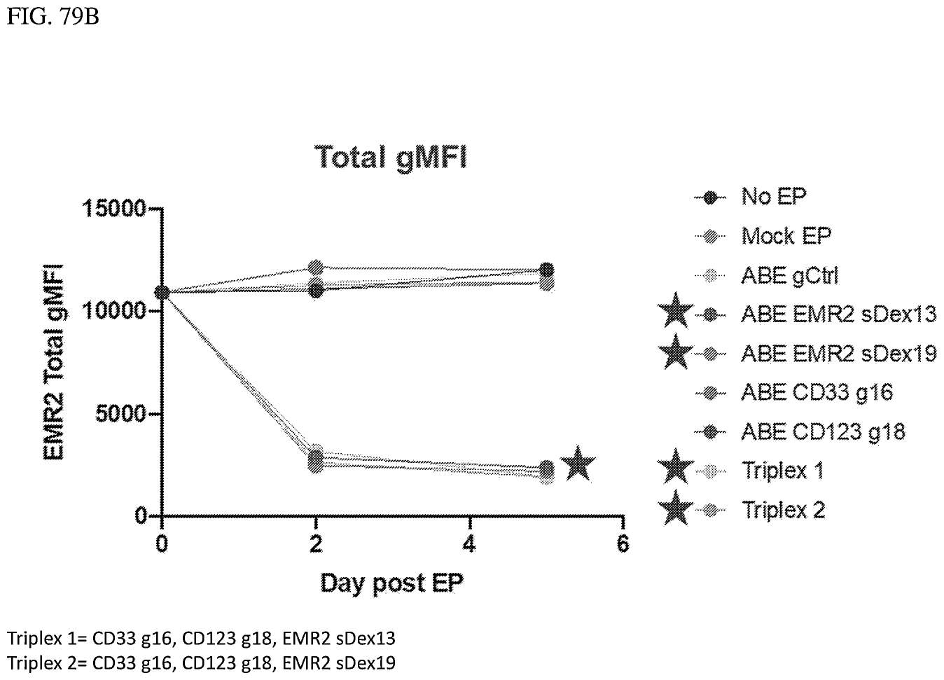

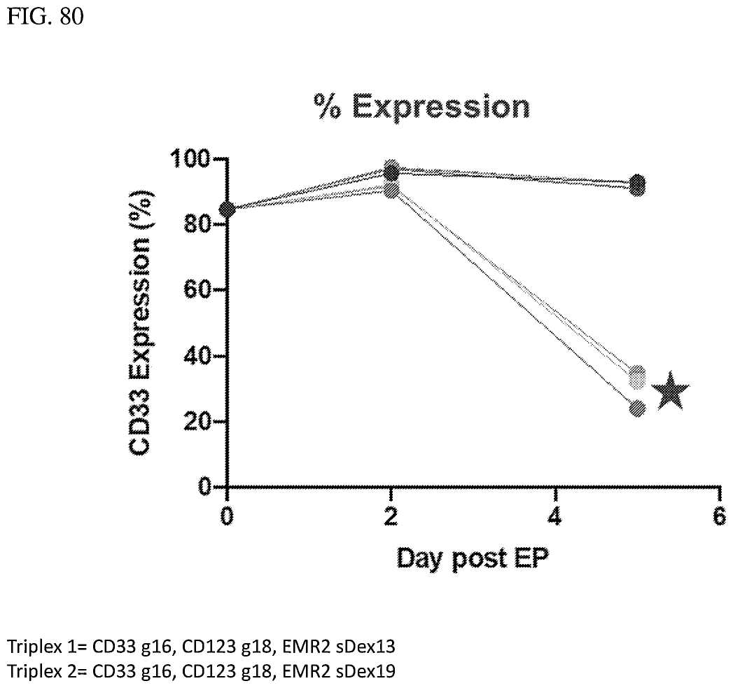

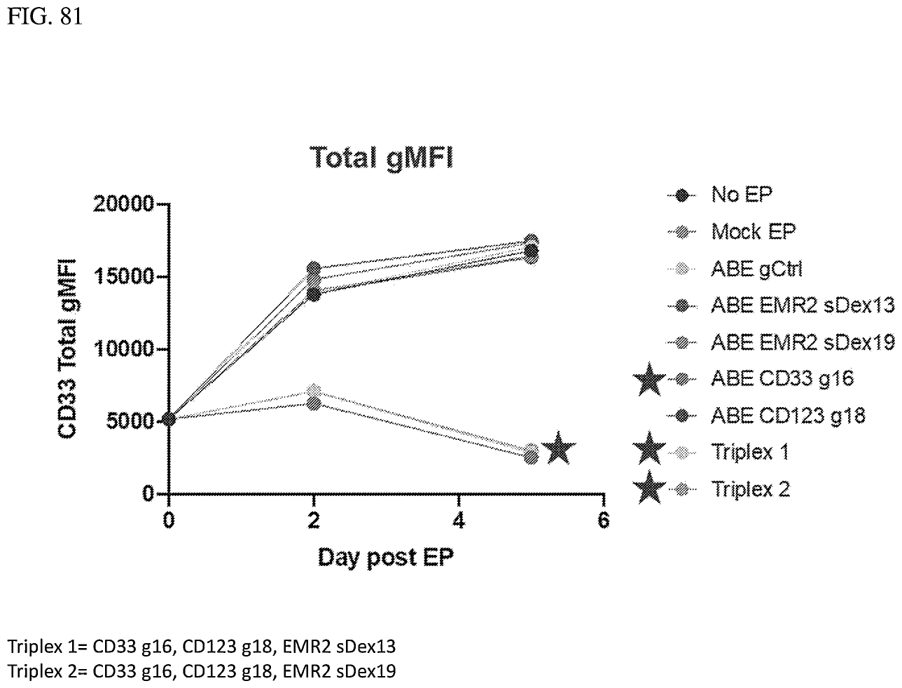

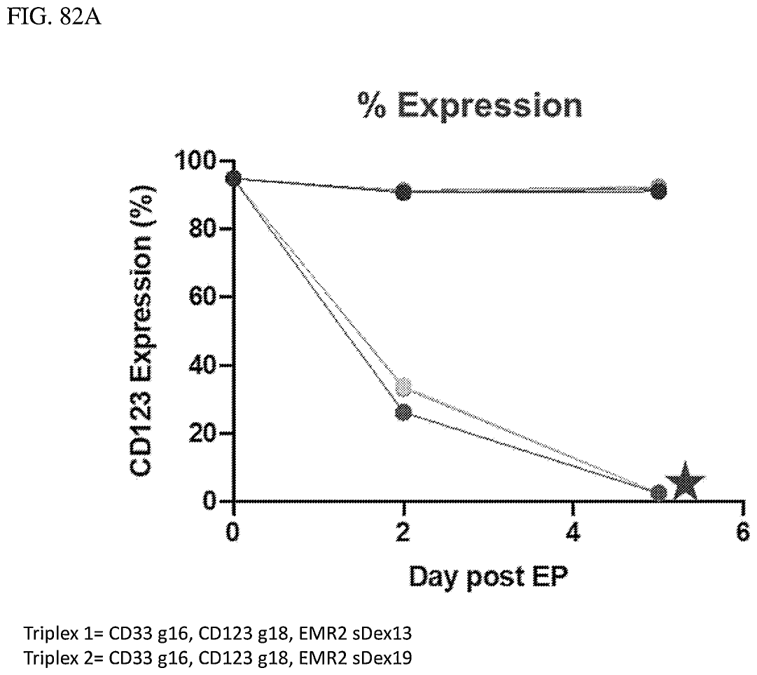

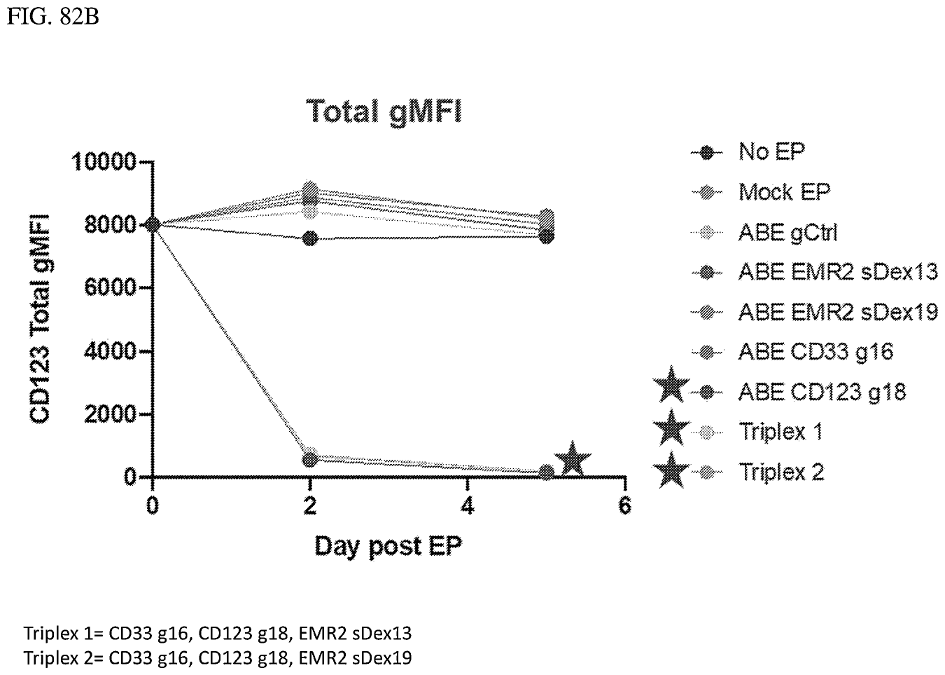

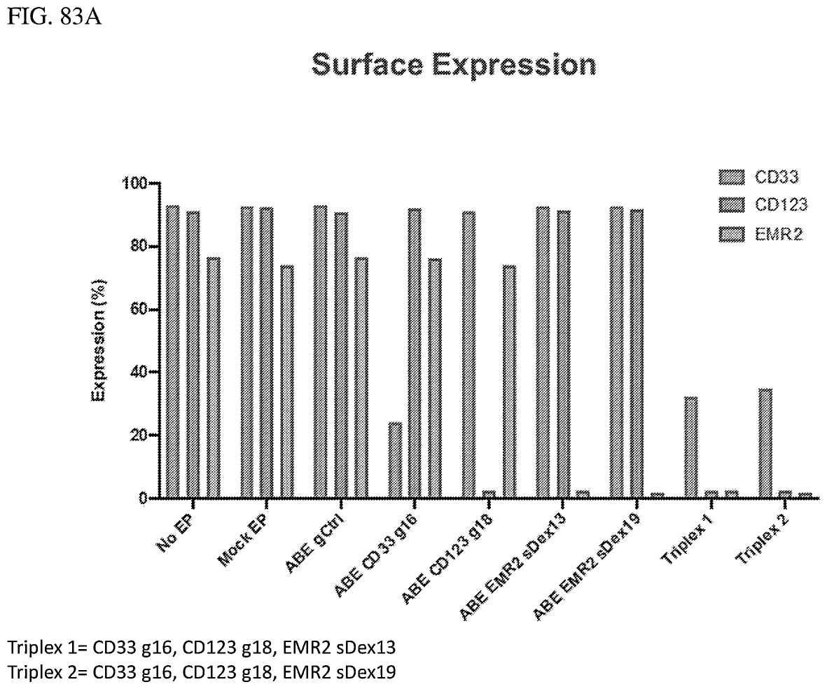

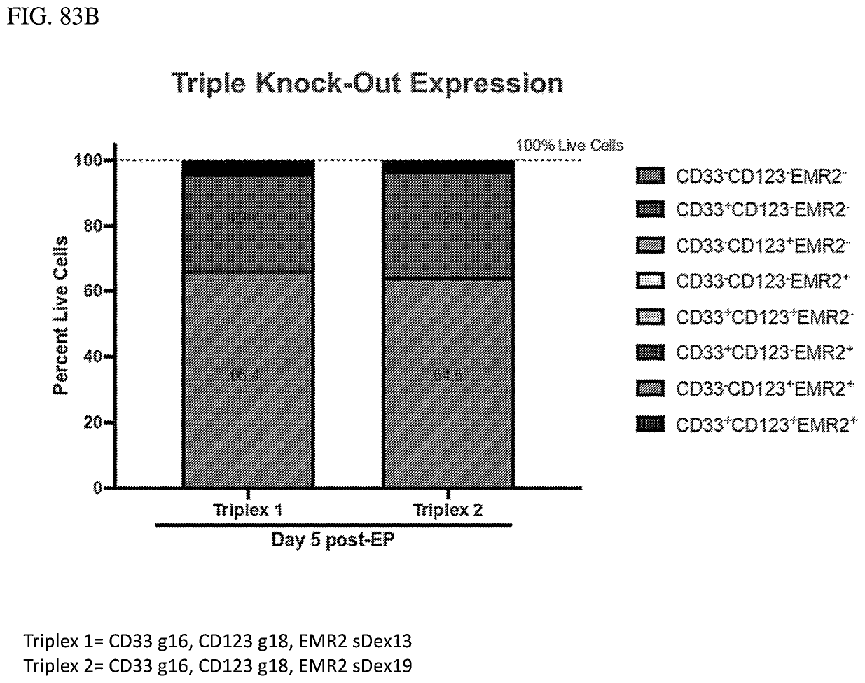

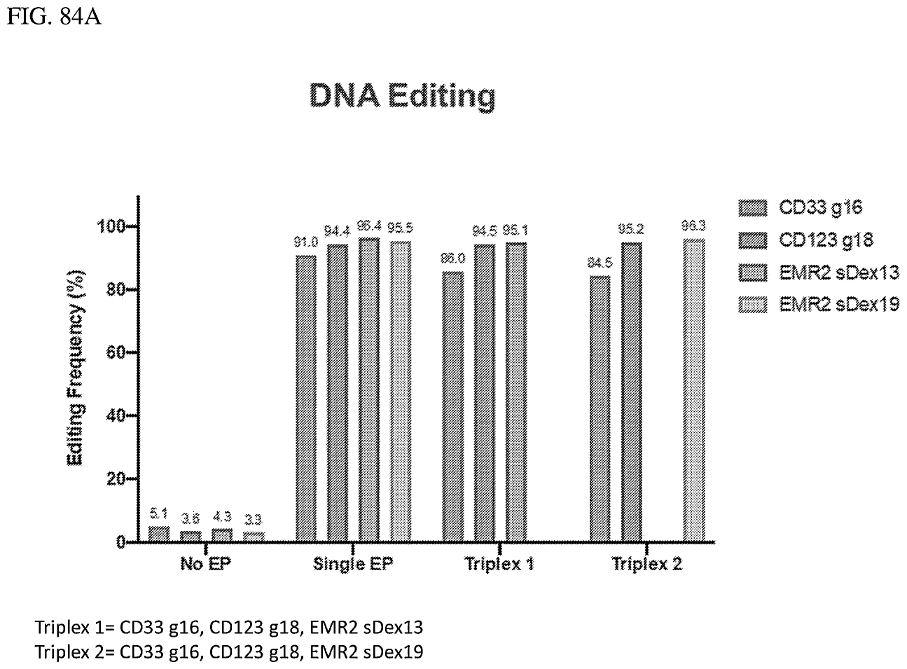

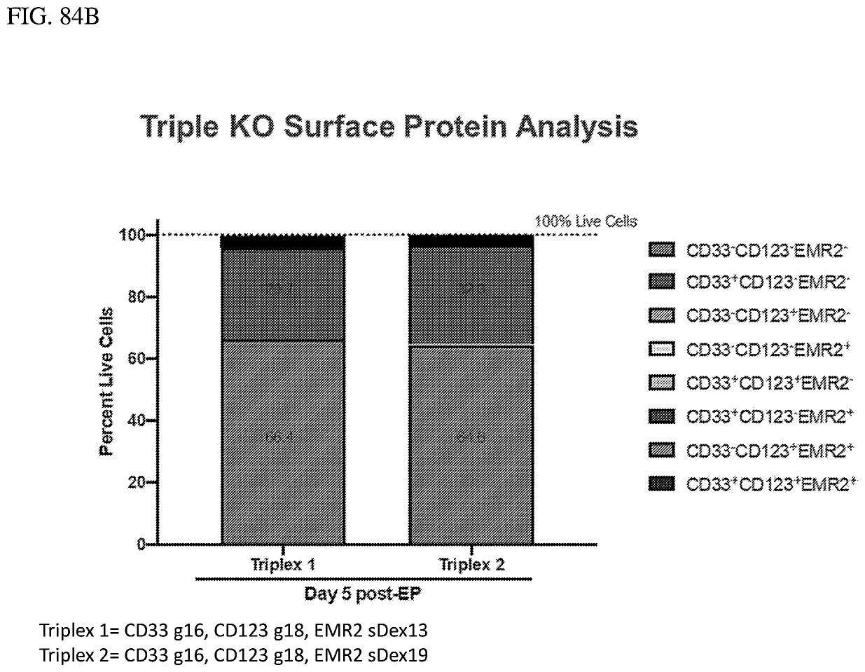

FIGS. 72-73 illustrates the experimental design and conditions. FIGS. 74A-74B show DNA editing frequency on day 2 and day 5 post EP, respectively. These data demonstrate>80% editing for CD33 g16 and >90% editing for CD123 g18, EMR2 sDex13 and EMR2 sDex19 at day 5 post EP. Similar editing was observed for CD123 g18, EMR2 sDex13 and EMR2 sDex19 in single and Triplex EP condition. A slight decrease in editing for CD33 g16 in Triplex compared to single EP (˜5% decrease) was also observed. Higher editing was observed at Day 5 compared to Day 2 across all guides and conditions. No off-target editing in CD97 for EMR2 sDex13 and EMR2 sDex19 was observed. FIGS. 75A-75B show DNA editing frequency on day 2 and day 5 post EP, respectively. These data demonstrate that the majority of editing for all guides in single and triplex conditions causes splice site disruption. EMR2 sDex13 shows ˜4% INDEL formation at Day 2 and Day 5 post-EP. FIGS. 76A-76B are schematics showing detailed substitution percentage summary in CD33sg16 and CD123sg18 groups, respectively. FIGS. 77A-77B are schematics showing detailed substitution percentage summary in EMR2sg13 and EMR2sg19 groups, respectively. FIGS. 78A-78C are schematics showing flow cytometry gating strategy. FIGS. 79A-79B show EMR2 surface protein expression and total gMFI, respectively. FIGS. 80-81 show CD33 surface protein expression and total gMFI, respectively. FIGS. 82A-82B show CD123 surface protein expression and total gMFI, respectively. FIGS. 83A-83B show CD33, CD123, and EMR2 surface protein expression, and Triple KO surface expression, respectively. FIGS. 84A-84B show CD33, CD123, and EMR2 DNA editing, and Triple KO surface protein analysis, respectively.

### Example 15: Evaluation of CLL-1 ABE Guides

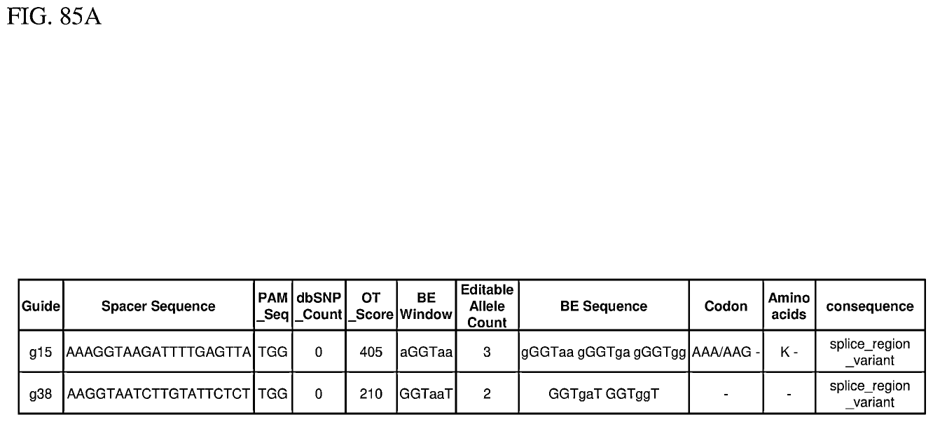

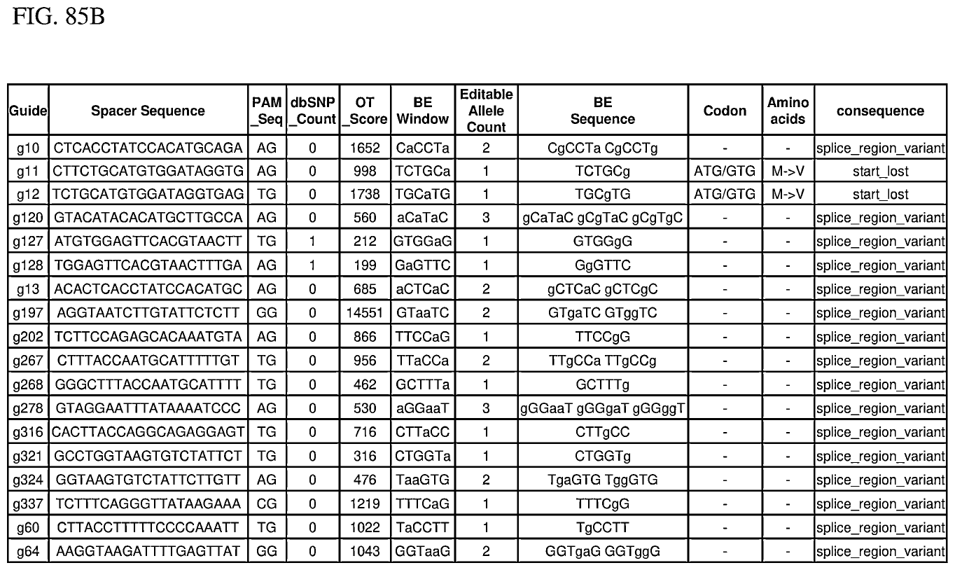

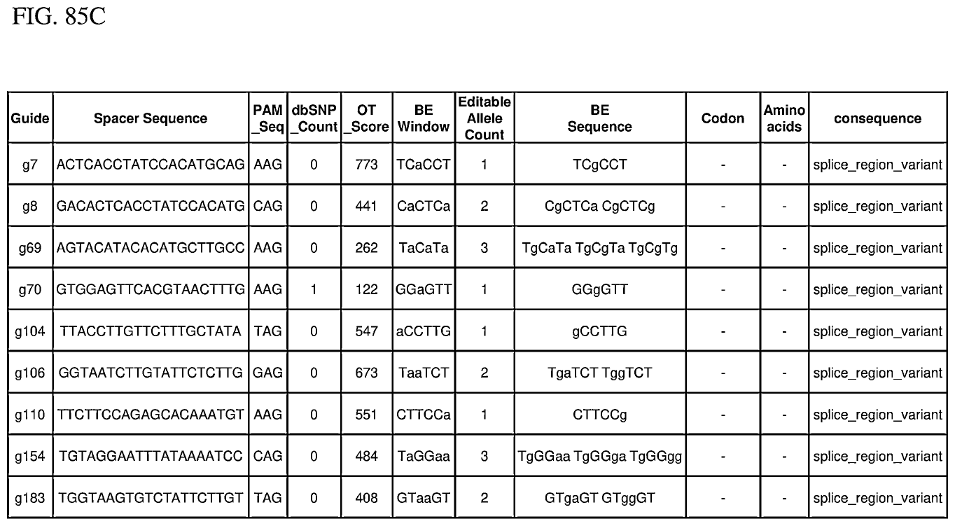

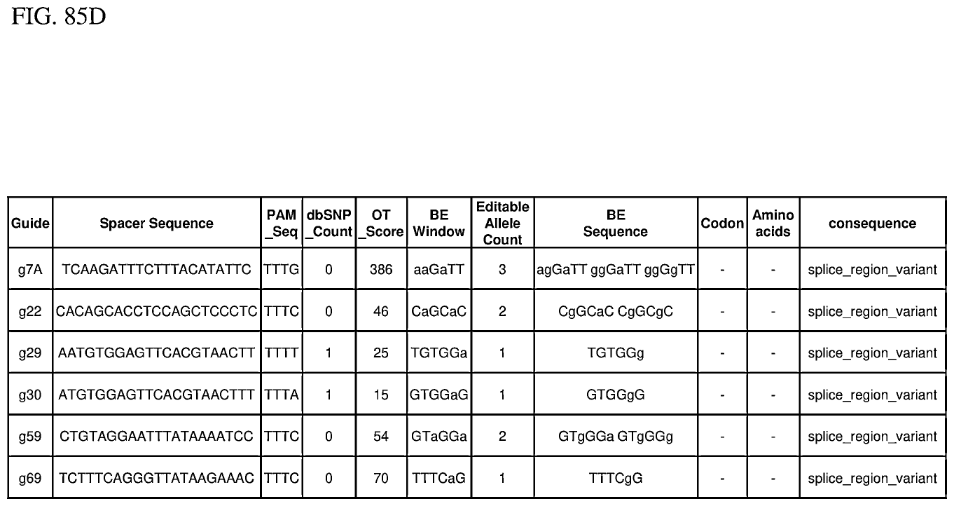

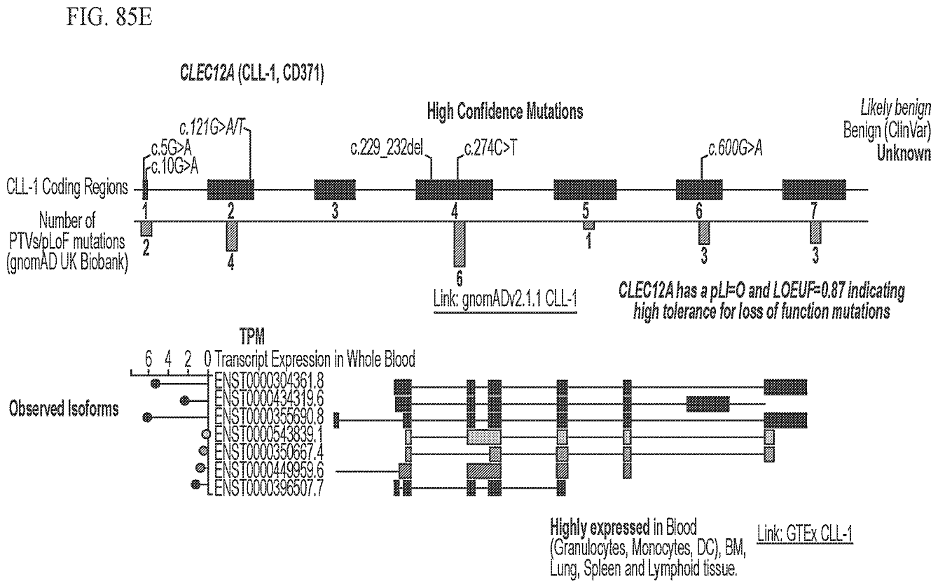

As shown in FIG. 85A, CLL-1 ABE Guides with SpCas9 NGG PAM were generated.

As shown in FIG. 85B, CLL-1 ABE Guides with Relaxed PAM (NG) were generated.

As shown in FIG. 85C, CLL-1 ABE Guides with Relaxed PAM (NRG) were generated.

As shown in FIG. 85D, CLL-1 ABE Guides with Cpf1 TTTN PAM were generated.

An overview of the CLL-1 gene is provided in FIG. 85E.

Both g15 and g38, below, disrupt the rare GTAA splice donor site to possibly cause CLL-1 Knockout.

| | | | | | | Editable | | | | |
|---|---|---|---|---|---|---|---|---|---|---|
| | Spacer | PAM_ | dbSNP_ | OT_ | BE | Allele | BE | | Amino | |
| Guide | Sequence | Seq | Count | Score | Window | Count | Sequence | Codon | acids | Consequence |
| g15 | AAAGGTAA | TGG | 0 | 405 | aGGTaa | 3 | gGGTaa | AAA/AAG | K - | Disrupts the |
| | GATTTTGA | | | | | | gGGTga | - | | rare GTAA |
| | GTTA | | | | | | gGGTgg | | | splice donor |
| | | | | | | | | | | site to |
| | | | | | | | | | | possibly |
| | | | | | | | | | | cause CLL-1 |
| | | | | | | | | | | Knockout |
| g38 | AAGGTAAT | TGG | 0 | 210 | GGTaaT | 2 | GGTgaT | — | — | Disrupts the |
| | CTTGTATT | | | | | | GGTggT | | | rare GTAA |
| | CTCT | | | | | | | | | splice donor |
| | | | | | | | | | | site to |
| | | | | | | | | | | possibly |
| | | | | | | | | | | cause CLL-1 |
| | | | | | | | | | | Knockout |

### Example 16: Evaluation of ABE CD33 g17 for Targeting Both CD33 and Siglec6

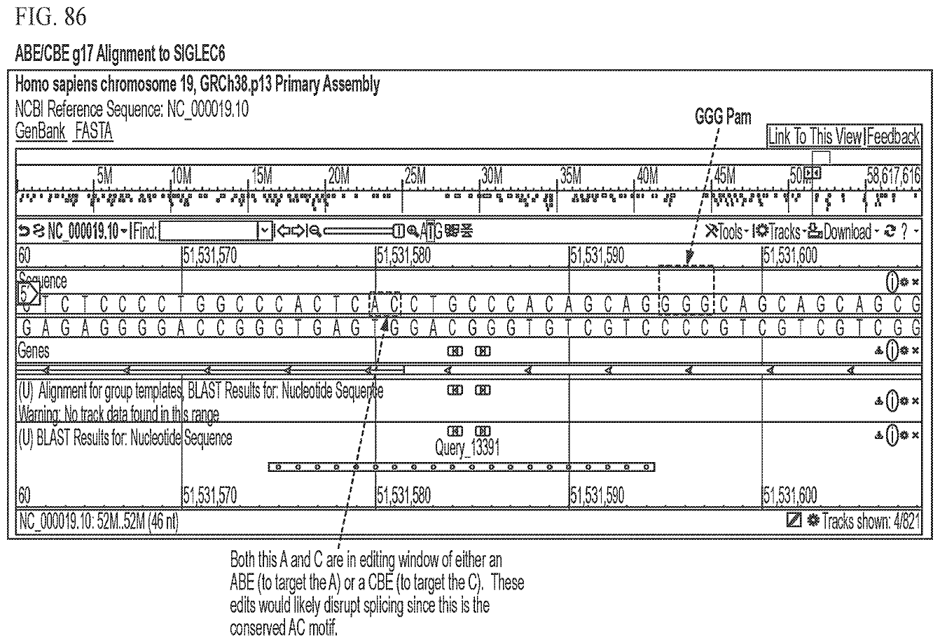

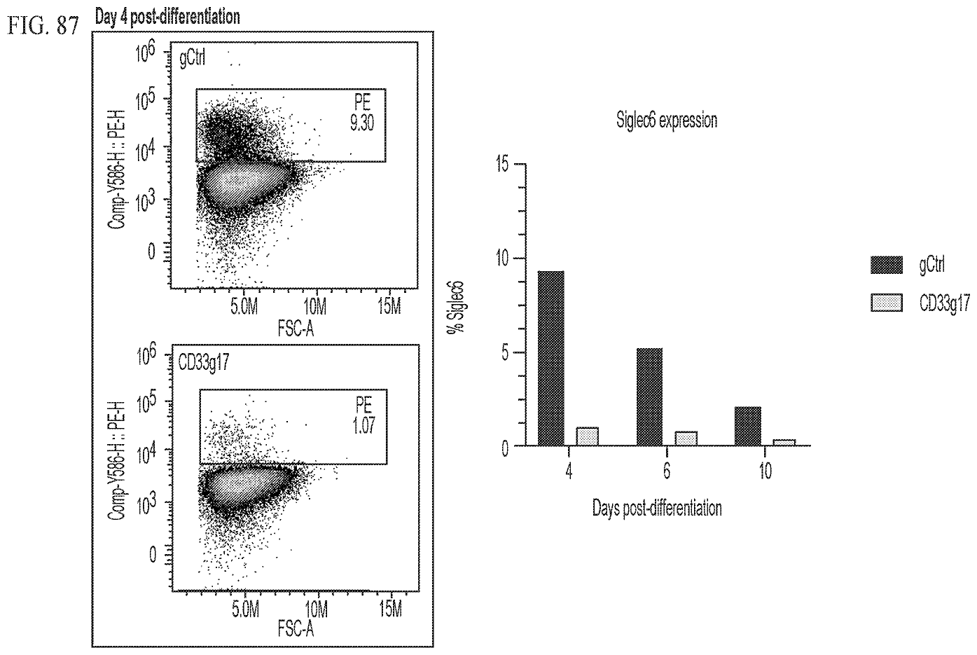

An analysis was performed on the impact of multiplex base editing on Siglec-6 from using a single guide RNA, i.e., CD33g17, in combination with a cytosine base editor (CBE) and/or an adenine base editor (ABE). Alignment of the ABE and CBE target sequences of CD33 g17 shows that CD33 g17 likely disrupts splicing in both CD33 (also known as “Siglec-3”) and Siglec-6. Specifically, as shown in FIG. 86, the CD33g17 guide would likely disrupt splicing in Siglec-6, because the ABE target sequence in Siglec-6 includes a GGG PAM similar to the GGG PAM present within the CBE target sequence in CD33. Further, the AC motif at the splice acceptor site is within the editing window of either an ABE or a CBE (e.g., the A is at position 6 from the 5′ end of the guide and the C is at position 7). The BE editing window is approximately between 4-8 bp from the 5′ end of the guide. Accordingly, conversion of the A to a G, or the C to a T, was predicted to result in disruption of splicing. Indeed, as shown in FIG. 87, Siglec-6 surface expression was decreased after editing with CD33g17 in combination with an adenine base editor (ABE). CD33 knockout was also observed.

### Multiplex CBE In Vitro Differentiation (IVD) Time Course Experiment.

Experiment Description: Identify the kinetics of multiplex base editing CD34+ cells by assessing protein KO, on-target editing, and transcript levels of CD33 and CLL1 over time throughout in vitro differentiation.

To identify the kinetics of multiplex base editing CD34+ cells protein knockout (KO), on-target editing, and transcript levels of CD33 and CLL1 were assessed over time throughout in vitro differentiation.

Briefly, CD34+ cells were edited with cytosine base editors (CBEs) targeting CD33 and CLL1 using gRNAs for these targets (CD33g8 and CLL1g3, and CD33g17 and CLL1g3). For comparison, cells were edited with Cas9 and the top gRNAs for CD33 and CLL1. These cells were subjected to in vitro differentiation for 2 weeks. At various time points throughout the experiment, cells will be collected to measure protein KO with flow cytometry, on-target editing, and transcript levels of CD33 and CLL1.

The experiment indicates whether multiplex editing CD33 and CLL1 using CBEs affects differentiation into monocytes or granulocytes. It will also helps identify the timepoints throughout differentiation for observing changes in protein expression, transcript levels, and on-target editing. Overall, these assays are used to determine of the kinetics of base editing in CD34+ cells.

### Siglec6 Antibody Testing.

Experiment Description: In vitro differentiated (IVD) HSCs edited with CD33g17 were evaluated for surface expression of Siglec-6. Optimal antibody clone selection and staining conditions for anti-Siglec-6 were evaluated with cell lines. These conditions were utilized to collect surface Siglec-6 levels on IVD differentiated eHSCs via flow cytometry. Flow data was analyzed with FlowJo software and the data plotted with Prism GraphPad.

Testing several antibodies and determine which had the best expression profile in Siglec6 positive and negative cell lines.

### Equivalents and Scope

Those skilled in the art will recognize, or be able to ascertain using no more than routine experimentation, many equivalents of the exemplary embodiments described herein. The scope of the present disclosure is not intended to be limited to the above description.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, it is to be understood that every possible individual element or subgroup of the elements is also disclosed, and that any element or subgroup of elements can be removed from the group. It is also noted that the term “comprising” is intended to be open and permits the inclusion of additional elements, features, or steps. It should be understood that, in general, where an embodiment, is referred to as comprising particular elements, features, or steps, embodiments, that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Aug. 28, 2019. When one gene or protein references a plurality of sequence accession numbers, all of the sequence variants are encompassed.

In addition, it is to be understood that any particular embodiment of the present disclosure may be explicitly excluded from any one or more of the claims. Where ranges are given, any value within the range may explicitly be excluded from any one or more of the claims. For purposes of brevity, all of the embodiments in which one or more elements, features, purposes, or aspects is excluded are not set forth explicitly herein.

1. A gRNA comprising a targeting domain which binds a target domain of Tables 1-19.

2. A gRNA comprising a targeting domain which binds a target domain comprising a nucleic acid sequence of any one of SEQ ID NOs: 1-2021.

3. A gRNA comprising a targeting domain capable of directing editing of a target domain of Tables 1-19.

4. A gRNA comprising a targeting domain, wherein the targeting domain comprises a nucleic acid sequence of any one of SEQ ID NOs: 1-2021.

5. The gRNA of any one of the preceding claims, which binds a target domain in a CD33 (Siglec-3) gene.

6. The gRNA of any one of the preceding claims, which binds a target domain in a CLL-1 gene.

7. The gRNA of any one of the preceding claims, which binds a target domain in a CD123 gene.

8. The gRNA of any one of the preceding claims, which binds a target domain in a CD327 (Siglec-6) gene.

9. The gRNA of any one of the preceding claims, which binds a target domain in a CD312 (EMR2) gene.

10. The gRNA of any one of the preceding claims, which binds a target domain in a CD327 (Siglec-6) gene.

11. The gRNA of any one of the preceding claims, wherein the targeting domain is configured to provide an editing event within the target domain under conditions suitable for the gRNA to form a complex with a gene editing enzyme, thus forming a gRNA:enzyme complex, and for the gRNA:enzyme complex to bind the target domain in a target nucleic acid molecule.

12. The gRNA of claim 11, wherein the gene editing enzyme comprises an endonuclease.

13. The gRNA of claim 12, wherein the endonuclease comprises a Cas endonuclease.

14. The gRNA of claim 12 or 13, wherein the endonuclease comprises a catalytically inactive Cas molecule.

15. The gRNA of any one of claims 12-14, wherein the endonuclease comprises a dead Cas (dCas).

16. The gRNA of claim 15, wherein the endonuclease comprises a dead Cas9 (dCas9).

17. The gRNA of any one of claims 12-14, wherein the endonuclease comprises a nickase (nCas).

18. The gRNA of claim 17, wherein the endonuclease comprises an nCas9.

19. The gRNA of any one of claims 12-18, wherein the endonuclease comprises a dCas or an nCas fused to one or more uracil glycosylase inhibitor (UGI) domains.

20. The gRNA of any one of claims 12-19, wherein the endonuclease comprises a dCas or an nCas fused to a base editor (BE).

21. The gRNA of any one of claims 12-20, wherein the endonuclease comprises a dCas or an nCas fused to an adenine base editor (ABE).

22. The gRNA of claim 21, wherein the ABE comprises an adenine deaminase enzyme.

23. The gRNA of any one of claims 12-20, wherein the endonuclease comprises a dCas or an nCas fused to a cytosine base editor (CBE).

24. The gRNA of claim 23, wherein the CBE comprises a cytidine deaminase enzyme.

25. The gRNA of any one of claims 11-24, wherein the nucleic acid molecule is comprised in the genomic DNA of a cell.

26. The gRNA of claim 25, wherein the cell is a mammalian cell.

27. The gRNA of claim 25 or 26, wherein the cell is a human cell.

28. The gRNA of claim 25 or 26, wherein the cell is a CD34+ cell.

29. The gRNA of claim 25 or 26, wherein the cell is a hematopoietic cell.

30. The gRNA of claim 25 or 26, wherein the cell is a hematopoietic stem cell.

31. The gRNA of claim 25 or 26, wherein the cell is a hematopoietic progenitor cell.

32. The gRNA of claim 25 or 26, wherein the cell is an immune effector cell.

33. The gRNA of claim 25 or 26, wherein the cell is a lymphocyte.

34. The gRNA of claim 25 or 26, wherein the cell is a T-lymphocyte.

35. The gRNA of claim 25 or 26, wherein the cell is a natural killer (NK) cell.

36. The gRNA of claim 25 or 26, wherein the cell is a stem cell.

37. The gRNA of claim 36, wherein, the stem cell is an embryonic stem cell (ESC), an induced pluripotent stem cell (iPSC), a mesenchymal stem cell, or a tissue-specific stem cell.

38. The gRNA of any one of claims 11-37, wherein the editing event comprises a chemical alteration to a nucleobase.

39. The gRNA of claim 38, wherein the editing event comprises the deamination of a cytosine.

40. The gRNA of claim 38, wherein the editing event comprises the deamination of an adenine.

41. The gRNA of claim 38, wherein the editing event comprises a nucleobase transition.

42. The gRNA of claim 38, wherein the editing event comprises a nucleobase transversion.

43. The gRNA of claim 38, wherein the editing event comprises converting a cytosine-guanine (C-G) base pair into a thymine-adenine (T-A) base pair within the target nucleic acid molecule.

44. The gRNA of claim 38, wherein the editing event comprises converting a thymine-adenine (T-A) base pair into a cytosine-guanine (C-G) base pair within the target nucleic acid molecule.

45. The gRNA of claim 38, wherein the editing event comprises introducing a premature STOP codon within the target nucleic acid molecule.

46. The gRNA of claim 38, wherein the editing event comprises introducing a splice site within the target nucleic acid molecule.

47. The gRNA of claim 38, wherein the editing event comprises disrupting a splice site within the target nucleic acid molecule.

48. The gRNA of any one of claims 38-47, wherein the target nucleic acid molecule comprises a chromosome or a genomic DNA molecule.

49. The gRNA of any one of claims 38-47, wherein the target nucleic acid molecule comprises the target domain.

50. The gRNA of claim 49, wherein the targeting domain of the gRNA base-pairs (in full or partial complementarity) with the sequence of the double-stranded target nucleic acid molecule that is complementary to the sequence of the target domain, which is the strand complementary to the strand that comprises a PAM sequence.

51. The gRNA of claim 50, wherein the targeting domain of the gRNA does not include the PAM sequence.

52. The gRNA of claim 50, wherein the location of the PAM may be 5′ or 3′ of the target domain sequence.

53. The gRNA of claim 51, wherein the position of the target nucleobases in the target domain is about 1, 2, 3, 4, 5, 6, 7, 8, 9, 10, 11, 12, 13, 14, 15, 16, 17, 18, 19, or 20 nucleobases from the PAM.

54. The gRNA of any one of claims 11-53, wherein the editing event reduces the activity of CD33 (Siglec-3) in a cell.

55. The gRNA of any one of claims 11-54, wherein the editing event reduces the expression level of a nucleic acid encoding CD33 (Siglec-3) in a cell.

56. The gRNA of any one of claims 11-55, wherein the editing event reduces the expression level of a CD33 (Siglec-3) protein in a cell.

57. The gRNA of any one of claims 11-56, wherein the editing event reduces or abolishes the expression of a full-length CD33 (Siglec-3) RNA or CD33 (Siglec-3) protein in a cell.

58. The gRNA of any one of claims 11-57, wherein the editing event reduces the activity of CLL-1 in a cell.

59. The gRNA of any one of claims 11-58, wherein the editing event reduces the expression level of a nucleic acid encoding CLL-1 in a cell.

60. The gRNA of any one of claims 11-59, wherein the editing event reduces the expression level of a CLL-1 protein in a cell.

61. The gRNA of any one of claims 11-60, wherein the editing event reduces or abolishes the expression of a full-length CLL-1 RNA or CLL-1 protein in a cell.

62. The gRNA of any one of claims 11-61, wherein the editing event reduces the activity of CD123 in a cell.

63. The gRNA of any one of claims 11-62, wherein the editing event reduces the expression level of a nucleic acid encoding CD123 in a cell

64. The gRNA of any one of claims 11-63, wherein the editing event reduces the expression level of a CD123 protein in a cell

65. The gRNA of any one of claims 11-64, wherein the editing event reduces or abolishes the expression of a full-length CD123 RNA or CD123 protein in a cell.

66. The gRNA of any one of claims 11-65, wherein the editing event reduces the activity of CD327 (Siglec-6) in a cell.

67. The gRNA of any one of claims 11-66, wherein the editing event reduces the expression level of a nucleic acid encoding CD327 (Siglec-6) in a cell.

68. The gRNA of any one of claims 11-67, wherein the editing event reduces the expression level of a CD327 (Siglec-6) protein in a cell.

69. The gRNA of any one of claims 11-68, wherein the editing event reduces or abolishes the expression of a full-length CD327 (Siglec-6) RNA or CD327 (Siglec-6) protein in a cell.

70. The gRNA of any one of claims 11-69, wherein the editing event reduces the activity of CD312 (EMR2) in a cell.

71. The gRNA of any one of claims 11-70, wherein the editing event reduces the expression level of a nucleic acid encoding CD312 (EMR2) in a cell

72. The gRNA of any one of claims 11-71, wherein the editing event reduces the expression level of a CD312 (EMR2) protein in a cell

73. The gRNA of any one of claims 11-72, wherein the editing event reduces or abolishes the expression of a full-length CD312 (EMR2) RNA or CD312 (EMR2) protein in a cell.

74. The gRNA of any one of claims 25-73, wherein the cell expresses a truncated version of a CD33 (Siglec-3), CLL-1, CD123, CD327 (Siglec-6), and/or CD312 (EMR2) RNA or protein.

75. The gRNA of claim 74, wherein the truncated version of the a CD33 (Siglec-3), CLL-1, CD123, CD327 (Siglec-6), and/or CD312 (EMR2) RNA or protein is expressed at a level equal to or greater than a level of a full-length a CD33 (Siglec-3), CLL-1, CD123, CD327 (Siglec-6), and/or CD312 (EMR2) RNA or protein in a non-edited cell.

76. The gRNA of claim 75, wherein a function or an activity of the truncated version of the a CD33 (Siglec-3), CLL-1, CD123, CD327 (Siglec-6), and/or CD312 (EMR2) RNA or protein is impaired or abolished.

77. The gRNA of claim 76, wherein the function or activity comprises binding to an antibody or a chimeric antigen receptor (CAR).

78. The gRNA of any one of the preceding claims, wherein the targeting domain is 16 nucleotides or more in length.

79. The gRNA of any one of the preceding claims, wherein the targeting domain is between about 16 to about 30 nucleotides in length.

80. The gRNA of any one of the preceding claims, wherein the targeting domain is 30 nucleotides in length.

81. The gRNA of any one of the preceding claims, wherein the targeting domain is 21 nucleotides in length.

82. The gRNA of any one of the preceding claims, wherein the targeting domain is 20 nucleotides in length.

83. The gRNA of any one of the preceding claims, wherein the targeting domain comprises a sequence of any one of SEQ ID NOs: 1-2021 or the reverse complement thereof, or a sequence having at least 90% or 95% identity thereto, or a sequence having no more than 1, 2, or 3 mutations relative thereto.

84. The gRNA of any one of the preceding claims, wherein the targeting domain comprises at least 16 consecutive nucleotides of any one of SEQ ID NOs: 1-2021, and/or base pairs or is complementary with at least 10 nucleotides of the target domain of any one of SEQ ID NOs: 1-2021.

85. The gRNA of any one of the preceding claims, which is a single guide RNA (sgRNA).

86. The gRNA of any one of the preceding claims, which comprises one or more chemical modifications.

87. The gRNA of any one of the preceding claims, which binds a base editor.

88. The gRNA of claim 87, wherein the base editor is a cytosine base editor (CBE).

89. The gRNA of claim 88, wherein the CBE is CBE1, CBE2, CBE3, or CBE4.

90. The gRNA of claim 88 or 89, wherein the CBE is selected from the group consisting of nCas9-2×UGI; BE4-rAPOBEC1; BE4-rAPOBEC1 K34A H122A; BE4-PpAPOBEC1; BE4-PpAPOBEC1 R33A; BE4-PpAPOBEC1 H122A; BE4-RrA3F; BE4-AmAPOBEC1; and BE4-SsAPOBEC3B.

91. The gRNA of any one of claims 88-90, wherein the CBE is a CBE-PpAPOBEC1 WT.

92. The gRNA of claim 87, wherein the base editor is an adenine base editor (ABE).

93. The gRNA of claim 92, wherein the ABE is ABE1, ABE2, ABE3, ABE4, ABE5, ABE6, ABE7, or ABE8.

94. The gRNA of claim 92 or 93, wherein the ABE is selected from the group consisting of ABE7.10-m; ABE7.10-d; ABE8.8-m; ABE8.8-d; ABE8.13-m; ABE8.13-d; ABE8.17-m; ABE8.17-d; ABE8.20-m; and ABE8.20-d.

95. The gRNA of any one of claims 92-94, wherein the ABE is an ABE8.

96. The gRNA of claim 87, wherein the base editor is a wildtype base editor.

97. A ribonucleoprotein (RNP) complex comprising a gRNA of any one of claims 1-96 and a base editor.

98. The gRNA of claim 97, wherein the base editor is a cytosine base editor (CBE).

99. The gRNA of claim 98, wherein the CBE is CBE1, CBE2, CBE3, or CBE4.

100. The gRNA of claim 98 or 99, wherein the CBE is selected from the group consisting of nCas9-2×UGI; BE4-rAPOBEC1; BE4-rAPOBEC1 K34A H122A; BE4-PpAPOBEC1; BE4-PpAPOBEC1 R33A; BE4-PpAPOBEC1 H122A; BE4-RrA3F; BE4-AmAPOBEC1; and BE4-SsAPOBEC3B.

101. The gRNA of claim 98, wherein the CBE is a CBE-PpAPOBEC1 WT.

102. The gRNA of claim 97, wherein the base editor is an adenine base editor (ABE).

103. The gRNA of claim 102, wherein the ABE is ABE1, ABE2, ABE3, ABE4, ABE5, ABE6, ABE7, or ABE8.

104. The gRNA of claim 102 or 103, wherein the ABE is selected from the group consisting of ABE7.10-m; ABE7.10-d; ABE8.8-m; ABE8.8-d; ABE8.13-m; ABE8.13-d; ABE8.17-m; ABE8.17-d; ABE8.20-m; and ABE8.20-d.

105. The gRNA of claim 102, wherein the ABE is an ABE8.

106. The gRNA of claim 97, wherein the base editor is a wildtype base editor.

107. A composition comprising a pre-formed complex comprising a base editor and a gRNA of any one of claims 1-96.

108. A mixture comprising an mRNA encoding a base editor and a gRNA of any one of claims 1-96.

109. A method for base editing, comprising:
contacting a target domain in a double-stranded DNA molecule with a complex comprising a base editor and a guide RNA (gRNA) of any one of claims 1-96,
wherein the base editor is a CBE or a ABE with a higher on-target editing efficiency as compared to a variant base editor.

110. The method of claim 109, wherein the base editor is a wildtype base editor.

111. The method of claim 110, wherein the wildtype base editor comprises BE4-PpAPOBEC.

112. The method of claim 109, wherein the variant base editor comprises BE4-PpAPOBEC1R33A.

113. The method of any one of claims 109-112, wherein the double-stranded DNA molecule is in a cell.

114. The method of claim 113, which comprises contacting the cell with the gRNA and an mRNA that encodes the base editor.

115. The method of claim 114, wherein the mRNA that encodes the base editor is chemically modified to improve expression of the encoded base editor.

116. The method of 115, wherein the chemically modified mRNA comprises a 5-methoxyuridine modification.

117. The method of claim 115, wherein the chemically modified mRNA comprises a N1-methylpseudouridine modification.

118. The method of any one of claims 114-117, which comprises contacting the cell with a ribonucleoprotein (RNP) complex comprising the gRNA and the base editor.

119. A method for multiplex base editing, comprising:
(i) providing a cell, and
(ii) introducing into the cell
(a) one or more guide RNAs (gRNAs) that target CD33 (Siglec-3), one or more gRNAs that target CLL-1, one or more gRNAs that target CD123, one or more gRNAs that target CD327 (Siglec-6), and/or one or more gRNAs that target CD312 (EMR2); and
(b) a base editor that binds the one or more gRNAs,
wherein the one or more gRNAs are configured to provide an editing event within the same or different target domains, thereby producing a genetically engineered cell.

120. A method of producing a genetically engineered cell, comprising:
(i) providing a cell, and
(ii) introducing into the cell
(a) one or more guide RNAs (gRNAs) that target CD33 (Siglec-3), one or more gRNAs that target CLL-1, one or more gRNAs that target CD123, one or more gRNAs that target CD327 (Siglec-6), and/or one or more gRNAs that target CD312 (EMR2); and
(b) a base editor that binds the one or more gRNAs,
wherein the one or more gRNAs are configured to provide an editing event within the same or different target domains, thereby producing a genetically engineered cell.

121. A method for multiplex base editing, comprising:
(i) providing a cell, and
(ii) introducing into the cell
(a) one or more guide RNAs (gRNAs) that target CD33;
(b) one or more gRNAs that target CLL-1 and/or one or more gRNAs that target CD123; and
(c) a base editor that binds the one or more gRNAs,
wherein the one or more gRNAs are configured to provide an editing event within different target domains, thereby producing a genetically engineered cell.

122. A method of producing a genetically engineered cell, comprising:
(i) providing a cell, and
(ii) introducing into the cell
(a) one or more guide RNAs (gRNAs) that target CD33;
(b) one or more gRNAs that target CLL-1 and/or one or more gRNAs that target CD123; and
(c) a base editor that binds the one or more gRNAs,
wherein the one or more gRNAs are configured to provide an editing event within different target domains, thereby producing a genetically engineered cell.

123. A method for multiplex base editing, comprising:
(i) providing a cell, and
(ii) introducing into the cell
(a) one or more guide RNAs (gRNAs) that target CD33 (Siglec-3);
(b) one or more gRNAs that target CLL-1, one or more gRNAs that target CD123, one or more gRNAs that target CD327 (Siglec-6), and/or one or more gRNAs that target CD312 (EMR2); and
(c) a base editor that binds the one or more gRNAs,
wherein the one or more gRNAs are configured to provide an editing event within the same or different target domains, thereby producing a genetically engineered cell.

124. A method of producing a genetically engineered cell, comprising:
(i) providing a cell, and
(ii) introducing into the cell
(a) one or more guide RNAs (gRNAs) that target CD33 (Siglec-3);
(b) one or more gRNAs that target CLL-1, one or more gRNAs that target CD123, one or more gRNAs that target CD327 (Siglec-6), and/or one or more gRNAs that target CD312 (EMR2); and
(c) a base editor that binds the one or more gRNAs,
wherein the one or more gRNAs are configured to provide an editing event within the same or different target domains, thereby producing a genetically engineered cell.

125. A method for triplex base editing, comprising:
(i) providing a cell, and
(ii) introducing into the cell
(a) a plurality of gRNAs configured to provide simultaneous editing events within at least three different genomic targets; and
(d) a base editor that binds the plurality of gRNAs, thereby producing a genetically engineered cell.

126. A method for triplex base editing, comprising:
(i) providing a cell, and
(ii) introducing into the cell
(a) one or more gRNAs that target CD33 (Siglec-3);
(b) one or more gRNAs that target CLL1;
(c) one or more gRNAs that target CD123; and
(d) a base editor that binds the one or more gRNAs,
wherein the one or more gRNAs are configured to provide simultaneous editing events within at least three different target domains, thereby producing a genetically engineered cell.

127. A method of producing a genetically engineered cell, comprising:
(i) providing a cell, and
(ii) introducing into the cell
(a) a plurality of gRNAs configured to provide simultaneous editing events within at least three different genomic targets; and
(d) a base editor that binds the plurality of gRNAs, thereby producing a genetically engineered cell.

128. A method of producing a genetically engineered cell, comprising:
(i) providing a cell, and
(ii) introducing into the cell
(a) one or more gRNAs that target CD33 (Siglec-3);
(b) one or more gRNAs that target CLL1;
(c) one or more gRNAs that target CD123; and
(d) a base editor that binds the one or more gRNAs,
wherein the one or more gRNAs are configured to provide simultaneous editing events within at least three different target domains, thereby producing a genetically engineered cell.

129. A method for quadruplex base editing, comprising:
(i) providing a cell, and
(ii) introducing into the cell
(a) a plurality of gRNAs configured to provide simultaneous editing events within at least four different genomic targets; and
(d) a base editor that binds the plurality of gRNAs, thereby producing a genetically engineered cell.

130. A method for quadruplex base editing, comprising:
(i) providing a cell, and
(ii) introducing into the cell
(a) one or more gRNAs that target CD33 (Siglec-3);
(b) one or more gRNAs that target CLL1;
(c) one or more gRNAs that target CD123;
(d) one or more gRNAs that target CD312 (EMR2);
(e) a base editor that binds the one or more gRNAs,
wherein the one or more gRNAs are configured to provide simultaneous editing events within at least four different target domains, thereby producing a genetically engineered cell.

131. A method of producing a genetically engineered cell, comprising:
(i) providing a cell, and
(ii) introducing into the cell
(a) a plurality of gRNAs configured to provide simultaneous editing events within at least four different genomic targets; and
(b) a base editor that binds the plurality of gRNAs, thereby producing a genetically engineered cell.

132. A method of producing a genetically engineered cell, comprising:
(i) providing a cell, and
(ii) introducing into the cell
(a) one or more gRNAs that target CD33 (Siglec-3);
(b) one or more gRNAs that target CLL1;
(c) one or more gRNAs that target CD123;
(d) one or more gRNAs that target CD312 (EMR2);
(e) a base editor that binds the one or more gRNAs,
wherein the one or more gRNAs are configured to provide simultaneous editing events within at least four different target domains, thereby producing a genetically engineered cell.

133. The method of any one of the preceding claims, wherein the one or more guide RNAs (gRNAs) comprise a gRNA of any one of claims 1-96.

134. The method of any one of the preceding claims, which results in the concurrent editing of one or more target domains within the same gene and/or within different genes.

135. The method of any one of the preceding claims, which results in the concurrent editing of two or more target domains within the same gene and/or within different genes.

136. The method of any one of the preceding claims, which results in the concurrent editing of three or more target domains within the same gene and/or within different genes.

137. The method of any one of the preceding claims, which results in the concurrent editing of four or more target domains within the same gene and/or within different genes.

138. The method of any one of the preceding claims, which results in the concurrent editing of one or more target domains within a CD33 (Siglec-3) gene, a CLL-1 gene, a CD123 gene, a CD327 (Siglec-6) gene, and/or a CD312 (EMR2) gene.

139. The method of any one of the preceding claims, wherein the one or more gRNAs that target CD33 (Siglec-3) are designed for use with a cytosine base editor (CBE) and/or an adenine base editor (ABE)

140. The method of any one of the preceding claims, wherein the one or more gRNAs that target CD33 (Siglec-3) are designed for use with a CBE.

141. The method of any one of the preceding claims, wherein the one or more gRNAs that target CD33 (Siglec-3) are designed for use with a ABE.

142. The method of any one of the preceding claims, wherein the one or more gRNAs that target CLL1 are designed for use with a cytosine base editor (CBE) and/or an adenine base editor (ABE).

143. The method of any one of the preceding claims, wherein the one or more gRNAs that target CLL1 are designed for use with a CBE.

144. The method of any one of the preceding claims, wherein the one or more gRNAs that target CLL1 are designed for use with a ABE.

145. The method of any one of the preceding claims, wherein the one or more gRNAs that target CD123 are designed for use with a CBE and/or an ABE.

146. The method of any one of the preceding claims, wherein the one or more gRNAs that target CD123 are designed for use with a CBE.

147. The method of any one of the preceding claims, wherein the one or more gRNAs that target CD123 are designed for use with a ABE.

148. The method of any one of the preceding claims, wherein the one or more gRNAs that target EMR2 are designed for use with a CBE and/or an ABE.

149. The method of any one of the preceding claims, wherein the one or more gRNAs that target EMR2 are designed for use with a CBE.

150. The method of any one of the preceding claims, wherein the one or more gRNAs that target EMR2 are designed for use with a ABE.

151. The method of any one of the preceding claims, which comprises contacting the cell with the one or more gRNAs and an mRNA that encodes the base editor.

152. The method of any one of the preceding claims, which comprises contacting the cell with a ribonucleoprotein (RNP) complex comprising the one or more gRNAs and the base editor.

153. The method of any one of the preceding claims, which comprises contacting the cell with the gRNA and an mRNA that encodes the base editor.

154. The method of any one of the preceding claims, wherein the mRNA that encodes the base editor is chemically modified to improve expression of the encoded base editor.

155. The method of any one of the preceding claims, wherein the chemically modified mRNA comprises a 5-methoxyuridine modification.

156. The method of any one of the preceding claims, wherein the chemically modified mRNA comprises a N1-methylpseudouridine modification.

157. The method of any one of the preceding claims, wherein the RNP is introduced into the cell via electroporation.

158. The method of any one of the preceding claims, wherein the base editor is a wildtype base editor.

159. The method of any one of the preceding claims, wherein the base editor is a cytosine base editor (CBE) and/or an adenine base editor (ABE)

160. The method of any one of the preceding claims, wherein only a CBE is introduced into the cell.

161. The method of any one of the preceding claims, wherein only an ABE is introduced into the cell.

162. The method of any one of the preceding claims, wherein both a CBE and an ABE are introduced into the cell.

163. The method of any one of the preceding claims, wherein a wildtype base editor is introduced into the cell, optionally, wherein a wildtype base editor targets a cytosine-guanine (C-G) base pair or a thymine-adenine (T-A) base pair with higher on-target editing efficiency as compared to a variant base editor.

164. The method of any one of the preceding claims, which results in a lower translocation risk as compared to a variant base editor, optionally, wherein the method results in 0% translocations, or an undetectable level of translocations, and an on-target editing efficiency of at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% or more for a modification in the endogenous CD33 (Siglec-3), CLL-1, CD123, CD327 (Siglec-6), and/or CD312 (EMR2) gene.

167. The method of any one of the preceding claims, wherein the cell comprises a hematopoietic stem cell or a progenitor cell.

168. A genetically engineered hematopoietic stem or progenitor cell, which is produced by a method of The method of any one of the preceding claims.

169. A cell population, comprising a plurality of the genetically engineered hematopoietic stem or progenitor cells of claim 168.

170. A cell population comprising a plurality of the genetically engineered hematopoietic stem or progenitor cells, wherein at least a portion of the cells comprise:
(i) an edited CD33 (Siglec-3) gene;
(ii) an edited CLL-1 gene;
(iii) an edited CD123 gene;
(iv) an edited CD327 (Siglec-6) gene;
(v) an edited CD312 (EMR2) gene;
(vi) an edited CD33 (Siglec-3) gene and an edited CLL-1 gene;
(vii) an edited CD33 (Siglec-3) gene and an edited CD123 gene;
(viii) an edited CD33 (Siglec-3) gene and an edited CD327 (Siglec-6) gene;
(ix) an edited CD33 (Siglec-3) gene and an edited CD312 (EMR2) gene;
(x) an edited CD33 (Siglec-3) gene, an edited CLL-1 gene, and an edited CD123 gene;
(xi) an edited CD33 (Siglec-3) gene, an edited CLL-1 gene, an edited CD123 gene, and an edited CD327 (Siglec-6) gene;
(xii) an edited CD33 (Siglec-3) gene, an edited CLL-1 gene, an edited CD123 gene, an edited CD327 (Siglec-6) gene, and an edited CD312 (EMR2) gene; or
(xiii) an edited CD33 (Siglec-3) gene, an edited CLL-1 gene, an edited CD123 gene, an edited CD327 (Siglec-6) gene, and/or an edited CD312 (EMR2) gene.

180. The cell population of any one of the preceding claims, wherein a CD33 (Siglec-3) gene comprises a stop codon or a mutated splice site, but not a frameshift mutation which is typically introduced by CRISPR nuclease-mediated nonhomologous end joining (NHEJ).

181. The cell population of any one of the preceding claims, wherein a CLL-1 gene comprises a stop codon or a mutated splice site, but not a frameshift mutation which is typically introduced by CRISPR nuclease-mediated nonhomologous end joining (NHEJ).

182. The cell population of any one of the preceding claims, wherein a CD123 gene comprises a stop codon or a mutated splice site, but not a frameshift mutation which is typically introduced by CRISPR nuclease-mediated nonhomologous end joining (NHEJ).

183. The cell population of any one of the preceding claims, wherein a CD327 (Siglec-6) gene comprises a stop codon or a mutated splice site, but not a frameshift mutation which is typically introduced by CRISPR nuclease-mediated nonhomologous end joining (NHEJ).

184. The cell population of any one of the preceding claims, wherein a CD312 (EMR2) gene comprises a stop codon or a mutated splice site, but not a frameshift mutation which is typically introduced by CRISPR nuclease-mediated nonhomologous end joining (NHEJ).

185. The cell population of any one of the preceding claims, which expresses less than 30% of the CD33 (Siglec-3) expressed by a wild-type counterpart cell population.

186. The cell population of any one of the preceding claims, which expresses less than 30% of the CLL-1 expressed by a wild-type counterpart cell population.

187. The cell population of any one of the preceding claims, which expresses less than 30% of the CD123 expressed by a wild-type counterpart cell population.

188. The cell population of any one of the preceding claims, which expresses less than 30% of the CD327 (Siglec-6) expressed by a wild-type counterpart cell population.

189. The cell population of any one of the preceding claims, which expresses less than 30% of the CD312 (EMR2) expressed by a wild-type counterpart cell population.

190. The cell population of any one of the preceding claims, wherein at least a portion of the cells have genetic editing at a gene encoding a lineage-specific cell-surface antigen other than CD33 (Siglec-3), CLL-1, CD123, CD327 (Siglec-6), or CD312 (EMR2).

191. The cell population of claim 190, wherein the gene encoding a lineage-specific cell surface antigen other than CD33 (Siglec-3), CLL-1, CD123, CD327 (Siglec-6), or CD312 (EMR2) is CD19, CD30, CD5, CD6, CD7, CD34, CD38, or BCMA.

192. A method, comprising administering to a subject in need thereof a cell population of any one of the preceding claims, optionally wherein the subject has a hematopoietic malignancy.

193. The method of any one of the preceding claims, wherein the hematopoietic malignancy comprises Hodgkin lymphoma, non-Hodgkin lymphoma, leukemia, or multiple myeloma.

194. The method of any one of the preceding claims, wherein the leukemia comprises acute myeloid leukemia (AML), acute lymphoid leukemia, chronic myelogenous leukemia, acute lymphoblastic leukemia or chronic lymphoblastic leukemia, and chronic lymphoid leukemia.

195. The method of any one of the preceding claims, wherein the hematopoietic malignancy comprises acute myeloid leukemia (AML).

196. The method of any one of the preceding claims, which further comprises administering to the subject an effective amount of an agent that targets CD33 (Siglec-3), CLL-1, CD123, CD327 (Siglec-6), and/or CD312 (EMR2), wherein the agent comprises an antigen binding fragment that binds CD33 (Siglec-3), CLL-1, CD123, CD327 (Siglec-6), and/or CD312 (EMR2).

197. The method of any one of the preceding claims, wherein the agent that targets CD33 (Siglec-3), CLL-1, CD123, CD327 (Siglec-6), and/or CD312 (EMR2) is an antibody or a chimeric antigen receptor (CAR).

198. A nucleic acid encoding the gRNA of any one of claims 1-96.

199. A kit or composition comprising: a) a gRNA of any one of claims 1-96, or a nucleic acid encoding the gRNA, and b) a second gRNA, or a nucleic acid encoding the second gRNA.


SEQUENCE LISTING (3033 sequences)

SEQ ID NO: 1 - DNA, 20 bp, synthetic construct
  misc_feature 1..20: note = Synthetic
  source 1..20: mol_type = other DNA
acctgtcaggtgaagttcgc

SEQ ID NO: 2 - DNA, 20 bp, synthetic construct
  misc_feature 1..20: note = Synthetic
  source 1..20: mol_type = other DNA
acaggcccaggacacagagc

SEQ ID NO: 3 - DNA, 20 bp, synthetic construct
  misc_feature 1..20: note = Synthetic
  source 1..20: mol_type = other DNA
tgacaaccaggagaagatcg

SEQ ID NO: 4 - DNA, 20 bp, synthetic construct
  misc_feature 1..20: note = Synthetic
  source 1..20: mol_type = other DNA
gttccacagaacccaacaac

SEQ ID NO: 5 - DNA, 20 bp, synthetic construct
  misc_feature 1..20: note = Synthetic
  source 1..20: mol_type = other DNA
gacaaccaggagaagatcgg

SEQ ID NO: 6 - DNA, 20 bp, synthetic construct
  misc_feature 1..20: note = Synthetic
  source 1..20: mol_type = other DNA
ctgacaaccaggagaagatc

SEQ ID NO: 7 - DNA, 20 bp, synthetic construct
  misc_feature 1..20: note = Synthetic
  source 1..20: mol_type = other DNA
ttcctacctgagccatctcc

SEQ ID NO: 8 - DNA, 20 bp, synthetic construct
  misc_feature 1..20: note = Synthetic
  source 1..20: mol_type = other DNA
acttacaggtgacgttgagc

SEQ ID NO: 9 - DNA, 20 bp, synthetic construct
  misc_feature 1..20: note = Synthetic
  source 1..20: mol_type = other DNA
ccactcacctgcccacagca

SEQ ID NO: 10 - DNA, 20 bp, synthetic construct
  misc_feature 1..20: note = Synthetic
  source 1..20: mol_type = other DNA
atgctcacatgaagaagatg

SEQ ID NO: 11 - DNA, 20 bp, synthetic construct
  misc_feature 1..20: note = Synthetic
  source 1..20: mol_type = other DNA
cactcacctgcccacagcag

SEQ ID NO: 12 - DNA, 20 bp, synthetic construct
  misc_feature 1..20: note = Synthetic
  source 1..20: mol_type = other DNA
caaatccatctccttggaca

SEQ ID NO: 13 - DNA, 20 bp, synthetic construct
  misc_feature 1..20: note = Synthetic
  source 1..20: mol_type = other DNA
ttatgccaaatccatctcct

SEQ ID NO: 14 - DNA, 20 bp, synthetic construct
  misc_feature 1..20: note = Synthetic
  source 1..20: mol_type = other DNA
attccagaactccagtgaga

SEQ ID NO: 15 - DNA, 20 bp, synthetic construct
  misc_feature 1..20: note = Synthetic
  source 1..20: mol_type = other DNA
cgccatacatgagagggagc

SEQ ID NO: 16 - DNA, 20 bp, synthetic construct
  misc_feature 1..20: note = Synthetic
  source 1..20: mol_type = other DNA
aacatggcaggagagtaaaa

SEQ ID NO: 17 - DNA, 20 bp, synthetic construct
  misc_feature 1..20: note = Synthetic
  source 1..20: mol_type = other DNA
gcgaacttcacctgacaggt

SEQ ID NO: 18 - DNA, 20 bp, synthetic construct
  misc_feature 1..20: note = Synthetic
  source 1..20: mol_type = other DNA
gctctgtgtcctgggcctgt

SEQ ID NO: 19 - DNA, 20 bp, synthetic construct
  misc_feature 1..20: note = Synthetic
  source 1..20: mol_type = other DNA
cgatcttctcctggttgtca

SEQ ID NO: 20 - DNA, 20 bp, synthetic construct
  misc_feature 1..20: note = Synthetic
  source 1..20: mol_type = other DNA
gttgttgggttctgtggaac

SEQ ID NO: 21 - DNA, 20 bp, synthetic construct
  misc_feature 1..20: note = Synthetic
  source 1..20: mol_type = other DNA
ccgatcttctcctggttgtc

SEQ ID NO: 22 - DNA, 20 bp, synthetic construct
  misc_feature 1..20: note = Synthetic
  source 1..20: mol_type = other DNA
gatcttctcctggttgtcag

SEQ ID NO: 23 - DNA, 20 bp, synthetic construct
  misc_feature 1..20: note = Synthetic
  source 1..20: mol_type = other DNA
ggagatggctcaggtaggaa

SEQ ID NO: 24 - DNA, 20 bp, synthetic construct
  misc_feature 1..20: note = Synthetic
  source 1..20: mol_type = other DNA
gctcaacgtcacctgtaagt

SEQ ID NO: 25 - DNA, 20 bp, synthetic construct
  misc_feature 1..20: note = Synthetic
  source 1..20: mol_type = other DNA
tgctgtgggcaggtgagtgg

SEQ ID NO: 26 - DNA, 20 bp, synthetic construct
  misc_feature 1..20: note = Synthetic
  source 1..20: mol_type = other DNA
catcttcttcatgtgagcat

SEQ ID NO: 27 - DNA, 20 bp, synthetic construct
  misc_feature 1..20: note = Synthetic
  source 1..20: mol_type = other DNA
catcttcttcatgtgagcat

SEQ ID NO: 28 - DNA, 20 bp, synthetic construct
  misc_feature 1..20: note = Synthetic
  source 1..20: mol_type = other DNA
tgtccaaggagatggatttg

SEQ ID NO: 29 - DNA, 20 bp, synthetic construct
  misc_feature 1..20: note = Synthetic
  source 1..20: mol_type = other DNA
aggagatggatttggcataa

SEQ ID NO: 30 - DNA, 20 bp, synthetic construct
  misc_feature 1..20: note = Synthetic
  source 1..20: mol_type = other DNA
tctcactggagttctggaat

SEQ ID NO: 31 - DNA, 20 bp, synthetic construct
  misc_feature 1..20: note = Synthetic
  source 1..20: mol_type = other DNA
gctccctctcatgtatggcg

SEQ ID NO: 32 - DNA, 20 bp, synthetic construct
  misc_feature 1..20: note = Synthetic
  source 1..20: mol_type = other DNA
ttttactctcctgccatgtt

SEQ ID NO: 33 - RNA, 20 nt, synthetic construct
  misc_feature 1..20: note = Synthetic
  source 1..20: mol_type = other RNA
acctgtcaggtgaagttcgc

SEQ ID NO: 34 - protein, 19 aa, synthetic construct
  REGION 1..19: note = Synthetic
GSTSSGSGKPGSGEGSTKG

SEQ ID NO: 35 - protein, 107 aa, synthetic construct
  REGION 1..107: note = Synthetic
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 36 - protein, 89 aa, synthetic construct
  REGION 1..89: note = Synthetic
LSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 37 - protein, 38 aa, synthetic construct
  REGION 1..38: note = Synthetic
CWLTKKKYSSSVHDPNGEYMFMRAVNTAKKSRLTDVTL

SEQ ID NO: 38 - protein, 97 aa, synthetic construct
  REGION 1..97: note = Synthetic
IEVMYPPPYLDNEKSNGTIIHVKGKHLCPSPLFPGPSKPFWVLVVVGGVLACYSLLVTVAFIIFWVRSKRSRLLHSDYMFMRAVNTAKKSRLTDVTL

SEQ ID NO: 39 - protein, 113 aa, synthetic construct
  REGION 1..113: note = Synthetic
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 40 - RNA, 20 nt, synthetic construct
  misc_feature 1..20: note = Synthetic
  source 1..20: mol_type = other RNA
acttacaggtgacgttgagc

SEQ ID NO: 41 - RNA, 20 nt, synthetic construct
  misc_feature 1..20: note = Synthetic
  source 1..20: mol_type = other RNA
ccactcacctgcccacagca

SEQ ID NO: 42 - RNA, 20 nt, synthetic construct
  misc_feature 1..20: note = Synthetic
  source 1..20: mol_type = other RNA
atgctcacatgaagaagatg

SEQ ID NO: 43 - RNA, 20 nt, synthetic construct
  misc_feature 1..20: note = Synthetic
  source 1..20: mol_type = other RNA
cactcacctgcccacagcag

SEQ ID NO: 44 - RNA, 20 nt, synthetic construct
  misc_feature 1..20: note = Synthetic
  source 1..20: mol_type = other RNA
caaatccatctccttggaca

SEQ ID NO: 45 - RNA, 20 nt, synthetic construct
  misc_feature 1..20: note = Synthetic
  source 1..20: mol_type = other RNA
ttatgccaaatccatctcct

SEQ ID NO: 46 - RNA, 20 nt, synthetic construct
  misc_feature 1..20: note = Synthetic
  source 1..20: mol_type = other RNA
attccagaactccagtgaga

SEQ ID NO: 47 - RNA, 20 nt, synthetic construct
  misc_feature 1..20: note = Synthetic
  source 1..20: mol_type = other RNA
cgccatacatgagagggagc

SEQ ID NO: 48 - RNA, 20 nt, synthetic construct
  misc_feature 1..20: note = Synthetic
  source 1..20: mol_type = other RNA
aacatggcaggagagtaaaa

SEQ ID NO: 49-63 - skipped (15 entries)

SEQ ID NO: 64 - DNA, 20 bp, synthetic construct
  misc_feature 1..20: note = Synthetic
  source 1..20: mol_type = other DNA
ccccaggactactcactcct

SEQ ID NO: 65 - DNA, 20 bp, synthetic construct
  misc_feature 1..20: note = Synthetic
  source 1..20: mol_type = other DNA
tttcttgagctgcagctggg

SEQ ID NO: 66 - DNA, 20 bp, synthetic construct
  misc_feature 1..20: note = Synthetic
  source 1..20: mol_type = other DNA
agttcccacatcctggtgcg

SEQ ID NO: 67-73 - skipped (7 entries)

SEQ ID NO: 74 - DNA, 20 bp, synthetic construct
  misc_feature 1..20: note = Synthetic
  source 1..20: mol_type = other DNA
ggtggctattgtttgcagtg

SEQ ID NO: 75-177 - skipped (103 entries)

SEQ ID NO: 178 - DNA, 23 bp, synthetic construct
  misc_feature 1..23: note = Synthetic
  source 1..23: mol_type = other DNA
acctgtcaggtgaagttcgctgg

SEQ ID NO: 179 - DNA, 23 bp, synthetic construct
  misc_feature 1..23: note = Synthetic
  source 1..23: mol_type = other DNA
tggccgggttctagagtgccagg

SEQ ID NO: 180 - DNA, 23 bp, synthetic construct
  misc_feature 1..23: note = Synthetic
  source 1..23: mol_type = other DNA
ggccgggttctagagtgccaggg

SEQ ID NO: 181 - DNA, 23 bp, synthetic construct
  misc_feature 1..23: note = Synthetic
  source 1..23: mol_type = other DNA
caccgaggagtgagtagtcctgg

SEQ ID NO: 182 - DNA, 23 bp, synthetic construct
  misc_feature 1..23: note = Synthetic
  source 1..23: mol_type = other DNA
tccagcgaacttcacctgacagg

SEQ ID NO: 183 - DNA, 20 bp, synthetic construct
  misc_feature 1..20: note = Synthetic
  source 1..20: mol_type = other DNA
gctgtggggagaggggttgt

SEQ ID NO: 184 - DNA, 20 bp, synthetic construct
  misc_feature 1..20: note = Synthetic
  source 1..20: mol_type = other DNA
ctgtggggagaggggttgtc

SEQ ID NO: 185 - DNA, 20 bp, synthetic construct
  misc_feature 1..20: note = Synthetic
  source 1..20: mol_type = other DNA
tggggaaacgagggtcagct

SEQ ID NO: 186 - DNA, 20 bp, synthetic construct
  misc_feature 1..20: note = Synthetic
  source 1..20: mol_type = other DNA
gggcccctgtggggaaacga

SEQ ID NO: 187 - DNA, 20 bp, synthetic construct
  misc_feature 1..20: note = Synthetic
  source 1..20: mol_type = other DNA
agggcccctgtggggaaacg

SEQ ID NO: 188 - DNA, 20 bp, synthetic construct
  misc_feature 1..20: note = Synthetic
  source 1..20: mol_type = other DNA
gctgaccctcgtttccccac

SEQ ID NO: 189 - DNA, 20 bp, synthetic construct
  misc_feature 1..20: note = Synthetic
  source 1..20: mol_type = other DNA
ctgaccctcgtttccccaca

SEQ ID NO: 190 - DNA, 20 bp, synthetic construct
  misc_feature 1..20: note = Synthetic
  source 1..20: mol_type = other DNA
tgaccctcgtttccccacag

SEQ ID NO: 191 - DNA, 20 bp, synthetic construct
  misc_feature 1..20: note = Synthetic
  source 1..20: mol_type = other DNA
ccatagccagggcccctgtg

SEQ ID NO: 192 - DNA, 20 bp, synthetic construct
  misc_feature 1..20: note = Synthetic
  source 1..20: mol_type = other DNA
gcatgtgacaggtgaggcac

SEQ ID NO: 193 - DNA, 20 bp, synthetic construct
  misc_feature 1..20: note = Synthetic
  source 1..20: mol_type = other DNA
tgaggcacaggcttcagaag

SEQ ID NO: 194 - DNA, 20 bp, synthetic construct
  misc_feature 1..20: note = Synthetic
  source 1..20: mol_type = other DNA
aggcttcagaagtggccgca

SEQ ID NO: 195 - DNA, 20 bp, synthetic construct
  misc_feature 1..20: note = Synthetic
  source 1..20: mol_type = other DNA
ggcttcagaagtggccgcaa

SEQ ID NO: 196 - DNA, 20 bp, synthetic construct
  misc_feature 1..20: note = Synthetic
  source 1..20: mol_type = other DNA
gtacccatgaacttcccttg

SEQ ID NO: 197 - DNA, 20 bp, synthetic construct
  misc_feature 1..20: note = Synthetic
  source 1..20: mol_type = other DNA
gtggccgcaagggaagttca

SEQ ID NO: 198 - DNA, 20 bp, synthetic construct
  misc_feature 1..20: note = Synthetic
  source 1..20: mol_type = other DNA
tggccgcaagggaagttcat

SEQ ID NO: 199 - DNA, 20 bp, synthetic construct
  misc_feature 1..20: note = Synthetic
  source 1..20: mol_type = other DNA
ggaagttcatgggtactgca

SEQ ID NO: 200 - DNA, 20 bp, synthetic construct
  misc_feature 1..20: note = Synthetic
  source 1..20: mol_type = other DNA
ttcatgggtactgcagggca

SEQ ID NO: 201 - DNA, 20 bp, synthetic construct
  misc_feature 1..20: note = Synthetic
  source 1..20: mol_type = other DNA
ctaaacccctcccagtacca

SEQ ID NO: 202 - DNA, 20 bp, synthetic construct
  misc_feature 1..20: note = Synthetic
  source 1..20: mol_type = other DNA
cactcacctgcccacagcag

SEQ ID NO: 203 - DNA, 20 bp, synthetic construct
  misc_feature 1..20: note = Synthetic
  source 1..20: mol_type = other DNA
ccctgctgtgggcaggtgag

SEQ ID NO: 204 - DNA, 20 bp, synthetic construct
  misc_feature 1..20: note = Synthetic
  source 1..20: mol_type = other DNA
tgggcaggtgagtggctgtg

SEQ ID NO: 205 - DNA, 20 bp, synthetic construct
  misc_feature 1..20: note = Synthetic
  source 1..20: mol_type = other DNA
ggtgagtggctgtggggaga

SEQ ID NO: 206 - DNA, 20 bp, synthetic construct
  misc_feature 1..20: note = Synthetic
  source 1..20: mol_type = other DNA
gtgagtggctgtggggagag

SEQ ID NO: 207 - DNA, 20 bp, synthetic construct
  misc_feature 1..20: note = Synthetic
  source 1..20: mol_type = other DNA
atccatagccagggcccctg

SEQ ID NO: 208 - DNA, 20 bp, synthetic construct
  misc_feature 1..20: note = Synthetic
  source 1..20: mol_type = other DNA
tccatagccagggcccctgt

SEQ ID NO: 209 - DNA, 20 bp, synthetic construct
  misc_feature 1..20: note = Synthetic
  source 1..20: mol_type = other DNA
tcgtttccccacaggggccc

SEQ ID NO: 210 - DNA, 20 bp, synthetic construct
  misc_feature 1..20: note = Synthetic
  source 1..20: mol_type = other DNA
tggctatggatccaaatttc

SEQ ID NO: 211 - DNA, 23 bp, synthetic construct
  misc_feature 1..23: note = Synthetic
  source 1..23: mol_type = other DNA
agaaatttggatccatagccagg

SEQ ID NO: 212 - DNA, 23 bp, synthetic construct
  misc_feature 1..23: note = Synthetic
  source 1..23: mol_type = other DNA
atccctggcactctagaacccgg

SEQ ID NO: 213 - DNA, 23 bp, synthetic construct
  misc_feature 1..23: note = Synthetic
  source 1..23: mol_type = other DNA
cctcactagacttgacccacagg

SEQ ID NO: 214-294 - skipped (81 entries)

SEQ ID NO: 295 - protein, 41 aa, synthetic construct
  REGION 1..41: note = Synthetic
KRGRKKLLYIFKQPFMRPVQTTQEEDGCSCRFPEEEEGGCE

SEQ ID NO: 296 - protein, 69 aa, synthetic construct
  REGION 1..69: note = Synthetic
TTTPAPRPPTPAPTIASQPLSLRPEACRPAAGGAVHTRGLDFACDIYIWAPLAGTCGVLLLSLVITLYC

SEQ ID NO: 297-300 - skipped (4 entries)

SEQ ID NO: 301 - DNA, 20 bp, synthetic construct
  misc_feature 1..20: note = Synthetic
  source 1..20: mol_type = other DNA
cccagaaattggcaaatttg

SEQ ID NO: 302 - DNA, 20 bp, synthetic construct
  misc_feature 1..20: note = Synthetic
  source 1..20: mol_type = other DNA
attccagaactccagtgaga

SEQ ID NO: 303 - DNA, 20 bp, synthetic construct
  misc_feature 1..20: note = Synthetic
  source 1..20: mol_type = other DNA
gagctatatagcaaagaaca

SEQ ID NO: 304 - DNA, 20 bp, synthetic construct
  misc_feature 1..20: note = Synthetic
  source 1..20: mol_type = other DNA
ggtggctattgtttgcagtg

SEQ ID NO: 305 - DNA, 20 bp, synthetic construct
  misc_feature 1..20: note = Synthetic
  source 1..20: mol_type = other DNA
ttgtttgcagtgtggtggag

SEQ ID NO: 306 - DNA, 20 bp, synthetic construct
  misc_feature 1..20: note = Synthetic
  source 1..20: mol_type = other DNA
tagctcacgacataatttgg

SEQ ID NO: 307 - DNA, 20 bp, synthetic construct
  misc_feature 1..20: note = Synthetic
  source 1..20: mol_type = other DNA
ggctattgtttgcagtgtgg

SEQ ID NO: 308 - DNA, 20 bp, synthetic construct
  misc_feature 1..20: note = Synthetic
  source 1..20: mol_type = other DNA
ggagaggttcctgatcttgt

SEQ ID NO: 309 - DNA, 20 bp, synthetic construct
  misc_feature 1..20: note = Synthetic
  source 1..20: mol_type = other DNA
tgaatatctccaacaagatc

SEQ ID NO: 310 - DNA, 20 bp, synthetic construct
  misc_feature 1..20: note = Synthetic
  source 1..20: mol_type = other DNA
gttgtagagaaatatttctc

SEQ ID NO: 311 - DNA, 20 bp, synthetic construct
  misc_feature 1..20: note = Synthetic
  source 1..20: mol_type = other DNA
caaatttgccaatttctggg

SEQ ID NO: 312 - DNA, 20 bp, synthetic construct
  misc_feature 1..20: note = Synthetic
  source 1..20: mol_type = other DNA
tctcactggagttctggaat

SEQ ID NO: 313 - DNA, 20 bp, synthetic construct
  misc_feature 1..20: note = Synthetic
  source 1..20: mol_type = other DNA
tgttctttgctatatagctc

SEQ ID NO: 314 - DNA, 20 bp, synthetic construct
  misc_feature 1..20: note = Synthetic
  source 1..20: mol_type = other DNA
cactgcaaacaatagccacc

SEQ ID NO: 315 - DNA, 20 bp, synthetic construct
  misc_feature 1..20: note = Synthetic
  source 1..20: mol_type = other DNA
ctccaccacactgcaaacaa

SEQ ID NO: 316 - DNA, 20 bp, synthetic construct
  misc_feature 1..20: note = Synthetic
  source 1..20: mol_type = other DNA
ccaaattatgtcgtgagcta

SEQ ID NO: 317 - DNA, 20 bp, synthetic construct
  misc_feature 1..20: note = Synthetic
  source 1..20: mol_type = other DNA
ccacactgcaaacaatagcc

SEQ ID NO: 318 - DNA, 20 bp, synthetic construct
  misc_feature 1..20: note = Synthetic
  source 1..20: mol_type = other DNA
acaagatcaggaacctctcc

SEQ ID NO: 319 - DNA, 20 bp, synthetic construct
  misc_feature 1..20: note = Synthetic
  source 1..20: mol_type = other DNA
gatcttgttggagatattca

SEQ ID NO: 320 - DNA, 20 bp, synthetic construct
  misc_feature 1..20: note = Synthetic
  source 1..20: mol_type = other DNA
gagaaatatttctctacaac

SEQ ID NO: 321 - RNA, 20 nt, synthetic construct
  misc_feature 1..20: note = Synthetic
  source 1..20: mol_type = other RNA
cccagaaattggcaaatttg

SEQ ID NO: 322 - RNA, 20 nt, synthetic construct
  misc_feature 1..20: note = Synthetic
  source 1..20: mol_type = other RNA
attccagaactccagtgaga

SEQ ID NO: 323 - RNA, 20 nt, synthetic construct
  misc_feature 1..20: note = Synthetic
  source 1..20: mol_type = other RNA
gagctatatagcaaagaaca

SEQ ID NO: 324 - RNA, 20 nt, synthetic construct
  misc_feature 1..20: note = Synthetic
  source 1..20: mol_type = other RNA
ggtggctattgtttgcagtg

SEQ ID NO: 325 - RNA, 20 nt, synthetic construct
  misc_feature 1..20: note = Synthetic
  source 1..20: mol_type = other RNA
ttgtttgcagtgtggtggag

SEQ ID NO: 326 - RNA, 20 nt, synthetic construct
  misc_feature 1..20: note = Synthetic
  source 1..20: mol_type = other RNA
tagctcacgacataatttgg

SEQ ID NO: 327 - RNA, 20 nt, synthetic construct
  misc_feature 1..20: note = Synthetic
  source 1..20: mol_type = other RNA
ggctattgtttgcagtgtgg

SEQ ID NO: 328 - RNA, 20 nt, synthetic construct
  misc_feature 1..20: note = Synthetic
  source 1..20: mol_type = other RNA
ggagaggttcctgatcttgt

SEQ ID NO: 329 - RNA, 20 nt, synthetic construct
  misc_feature 1..20: note = Synthetic
  source 1..20: mol_type = other RNA
tgaatatctccaacaagatc

SEQ ID NO: 330 - RNA, 20 nt, synthetic construct
  misc_feature 1..20: note = Synthetic
  source 1..20: mol_type = other RNA
gttgtagagaaatatttctc

SEQ ID NO: 331-339 - skipped (9 entries)

SEQ ID NO: 340 - DNA, 20 bp, synthetic construct
  misc_feature 1..20: note = Synthetic
  source 1..20: mol_type = other DNA
atataatcaactcctctgcc

SEQ ID NO: 341 - DNA, 20 bp, synthetic construct
  misc_feature 1..20: note = Synthetic
  source 1..20: mol_type = other DNA
ggcagaggagttgattatat

SEQ ID NO: 342 - DNA, 20 bp, synthetic construct
  misc_feature 1..20: note = Synthetic
  source 1..20: mol_type = other DNA
aatagacacttaccaggcag

SEQ ID NO: 343 - DNA, 20 bp, synthetic construct
  misc_feature 1..20: note = Synthetic
  source 1..20: mol_type = other DNA
ctgcctggtaagtgtctatt

SEQ ID NO: 344 - RNA, 20 nt, synthetic construct
  misc_feature 1..20: note = Synthetic
  source 1..20: mol_type = other RNA
atataatcaactcctctgcc

SEQ ID NO: 345 - RNA, 20 nt, synthetic construct
  misc_feature 1..20: note = Synthetic
  source 1..20: mol_type = other RNA
aatagacacttaccaggcag

SEQ ID NO: 346 - DNA, 4764 bp, synthetic construct
  misc_feature 1..4764: note = Synthetic
  source 1..4764: mol_type = other DNA
atgtccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtactcgacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgactctggtggttcttctggtggttctagcggcagcgagactcccgggacctcagagtccgccacacccgaaagttctggtggttcttctggtggttctgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacgagggagctgataagcgcaccgccgatggttccgagttcgaaagccccaagaagaagaggaaagtc

SEQ ID NO: 347 - DNA, 5490 bp, synthetic construct
  misc_feature 1..5490: note = Synthetic
  source 1..5490: mol_type = other DNA
atgacctctgagaagggccctagcacaggcgaccccaccctgcggcggagaatcgagagctgggagttcgacgtgttctacgaccctagagaactggccaaggaaacctgcctgctgtacgagatcaagtggggcatgagcagaaagatctggcggagctctggcaagaacaccaccaaccacgtggaagtgaatttcatcaagaagttcaccagcgagagaaggttccacagcagcatcagctgcagcatcacctggttcctgagctggtccccttgctgggaatgcagccaggccatcagagagttcctgagccaacaccccggagtgacactggtgatctacgtggccagactgttctggcacatggaccagagaaacagacagggcctgagagatctggtcaacagcggcgtgactatccagatcatgcgggccagcgagtactaccactgttggcggaacttcgtgaactacccccccggcgatgaggcccactggcctcagtaccctcctctgtggatgatgctgtacgccctggaactgcactgcatcatcctgtctctgcctccatgtctgaagatctctagaagatggcagaaccacctggccttcttcagactgcacctgcagaattgccactaccagaccatccccccccacatcctgctggctacaggcctgatccacccttctgtgacctggagacttaagagcggaggatctagcggcggctctagcggatctgagacacctggcacaagcgagtctgccacacctgagagtagcggcggatcttctggtggctctgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggtggaagcggaggatctggcggcagcaccaatctgagcgacatcatcgagaaagagacaggcaagcagctggtcatccaagagtccatcctgatgctgcctgaagaggtggaagaagtgatcggcaacaagcccgagtccgacatcctggtgcacaccgcctacgatgagagcaccgacgagaacgtgatgctgctgacctctgacgcccctgagtacaagccttgggctctcgtgatccaggacagcaacggcgagaacaagatcaagatgctgagcggcggctctggtggctctggcggatctacaaacctgtccgatattattgagaaagaaaccgggaaacagctcgtgattcaagagtctattctcatgctcccggaagaagtcgaggaagtcattggaaacaagcctgagagcgatattctggtccatacagcctacgacgagtctaccgatgagaatgtcatgctcctcaccagcgacgctcccgagtataagccatgggcacttgtcattcaggactccaatggggaaaacaaaatcaaaatgctcccaaagaaaaaacgcaaggtg

SEQ ID NO: 348 - DNA, 5490 bp, synthetic construct
  misc_feature 1..5490: note = Synthetic
  source 1..5490: mol_type = other DNA
atgacctctgagaagggccctagcacaggcgaccccaccctgcggcggagaatcgagagctgggagttcgacgtgttctacgaccctagagaactgagaaaggaaacctgcctgctgtacgagatcaagtggggcatgagcagaaagatctggcggagctctggcaagaacaccaccaaccacgtggaagtgaatttcatcaagaagttcaccagcgagagaaggttccacagcagcatcagctgcagcatcacctggttcctgagctggtccccttgctgggaatgcagccaggccatcagagagttcctgagccaacaccccggagtgacactggtgatctacgtggccagactgttctggcacatggaccagagaaacagacagggcctgagagatctggtcaacagcggcgtgactatccagatcatgcgggccagcgagtactaccactgttggcggaacttcgtgaactacccccccggcgatgaggcccactggcctcagtaccctcctctgtggatgatgctgtacgccctggaactgcactgcatcatcctgtctctgcctccatgtctgaagatctctagaagatggcagaaccacctggccttcttcagactgcacctgcagaattgccactaccagaccatccccccccacatcctgctggctacaggcctgatccacccttctgtgacctggagacttaagagcggaggatctagcggcggctctagcggatctgagacacctggcacaagcgagtctgccacacctgagagtagcggcggatcttctggtggctctgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggtggaagcggaggatctggcggcagcaccaatctgagcgacatcatcgagaaagagacaggcaagcagctggtcatccaagagtccatcctgatgctgcctgaagaggtggaagaagtgatcggcaacaagcccgagtccgacatcctggtgcacaccgcctacgatgagagcaccgacgagaacgtgatgctgctgacctctgacgcccctgagtacaagccttgggctctcgtgatccaggacagcaacggcgagaacaagatcaagatgctgagcggcggctctggtggctctggcggatctacaaacctgtccgatattattgagaaagaaaccgggaaacagctcgtgattcaagagtctattctcatgctcccggaagaagtcgaggaagtcattggaaacaagcctgagagcgatattctggtccatacagcctacgacgagtctaccgatgagaatgtcatgctcctcaccagcgacgctcccgagtataagccatgggcacttgtcattcaggactccaatggggaaaacaaaatcaaaatgctcccaaagaaaaaacgcaaggtg

SEQ ID NO: 349 - protein, 1588 aa, synthetic construct
  REGION 1..1588: note = Synthetic
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 350 - protein, 1830 aa, synthetic construct
  REGION 1..1830: note = Synthetic
MTSEKGPSTGDPTLRRRIESWEFDVFYDPRELAKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWRLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLPKKKRKV

SEQ ID NO: 351 - protein, 1830 aa, synthetic construct
  REGION 1..1830: note = Synthetic
MTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWRLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLPKKKRKV

SEQ ID NO: 352 - RNA, 20 nt, synthetic construct
  misc_feature 1..20: note = Synthetic
  source 1..20: mol_type = other RNA
acaggcccaggacacagagc

SEQ ID NO: 353 - RNA, 20 nt, synthetic construct
  misc_feature 1..20: note = Synthetic
  source 1..20: mol_type = other RNA
tgacaaccaggagaagatcg

SEQ ID NO: 354 - RNA, 20 nt, synthetic construct
  misc_feature 1..20: note = Synthetic
  source 1..20: mol_type = other RNA
gttccacagaacccaacaac

SEQ ID NO: 355 - RNA, 20 nt, synthetic construct
  misc_feature 1..20: note = Synthetic
  source 1..20: mol_type = other RNA
gacaaccaggagaagatcgg

SEQ ID NO: 356 - RNA, 20 nt, synthetic construct
  misc_feature 1..20: note = Synthetic
  source 1..20: mol_type = other RNA
ctgacaaccaggagaagatc

SEQ ID NO: 357 - RNA, 20 nt, synthetic construct
  misc_feature 1..20: note = Synthetic
  source 1..20: mol_type = other RNA
ttcctacctgagccatctcc

SEQ ID NO: 358-399 - skipped (42 entries)

SEQ ID NO: 400 - DNA, 20 bp, synthetic construct
  misc_feature 1..20: note = Synthetic
  source 1..20: mol_type = other DNA
atgcagaagtcaaatttgaa

SEQ ID NO: 401 - DNA, 20 bp, synthetic construct
  misc_feature 1..20: note = Synthetic
  source 1..20: mol_type = other DNA
tcaaatttgacttctgcatg

SEQ ID NO: 402 - DNA, 20 bp, synthetic construct
  misc_feature 1..20: note = Synthetic
  source 1..20: mol_type = other DNA
tttgacttctgcatgtggat

SEQ ID NO: 403 - DNA, 20 bp, synthetic construct
  misc_feature 1..20: note = Synthetic
  source 1..20: mol_type = other DNA
ggataggtgagtgtcttttg

SEQ ID NO: 404 - DNA, 20 bp, synthetic construct
  misc_feature 1..20: note = Synthetic
  source 1..20: mol_type = other DNA
tttccatctcactggagttc

SEQ ID NO: 405 - DNA, 20 bp, synthetic construct
  misc_feature 1..20: note = Synthetic
  source 1..20: mol_type = other DNA
tgggattttttccatctcac

SEQ ID NO: 406 - DNA, 20 bp, synthetic construct
  misc_feature 1..20: note = Synthetic
  source 1..20: mol_type = other DNA
atggaaaaaatcccagaaat

SEQ ID NO: 407 - DNA, 20 bp, synthetic construct
  misc_feature 1..20: note = Synthetic
  source 1..20: mol_type = other DNA
atcccagaaattggcaaatt

SEQ ID NO: 408 - DNA, 20 bp, synthetic construct
  misc_feature 1..20: note = Synthetic
  source 1..20: mol_type = other DNA
tcccagaaattggcaaattt

SEQ ID NO: 409 - DNA, 20 bp, synthetic construct
  misc_feature 1..20: note = Synthetic
  source 1..20: mol_type = other DNA
ccccaaatttgccaatttct

SEQ ID NO: 410 - DNA, 20 bp, synthetic construct
  misc_feature 1..20: note = Synthetic
  source 1..20: mol_type = other DNA
tccccaaatttgccaatttc

SEQ ID NO: 411 - DNA, 20 bp, synthetic construct
  misc_feature 1..20: note = Synthetic
  source 1..20: mol_type = other DNA
attggcaaatttggggaaaa

SEQ ID NO: 412 - DNA, 20 bp, synthetic construct
  misc_feature 1..20: note = Synthetic
  source 1..20: mol_type = other DNA
aaaggtaagattttgagtta

SEQ ID NO: 413 - DNA, 20 bp, synthetic construct
  misc_feature 1..20: note = Synthetic
  source 1..20: mol_type = other DNA
agagggagctggaggtgctg

SEQ ID NO: 414 - DNA, 20 bp, synthetic construct
  misc_feature 1..20: note = Synthetic
  source 1..20: mol_type = other DNA
ctccagctccctctcatgta

SEQ ID NO: 415 - DNA, 20 bp, synthetic construct
  misc_feature 1..20: note = Synthetic
  source 1..20: mol_type = other DNA
catacatgagagggagctgg

SEQ ID NO: 416 - DNA, 20 bp, synthetic construct
  misc_feature 1..20: note = Synthetic
  source 1..20: mol_type = other DNA
cgccatacatgagagggagc

SEQ ID NO: 417 - DNA, 20 bp, synthetic construct
  misc_feature 1..20: note = Synthetic
  source 1..20: mol_type = other DNA
gctggacgccatacatgaga

SEQ ID NO: 418 - DNA, 20 bp, synthetic construct
  misc_feature 1..20: note = Synthetic
  source 1..20: mol_type = other DNA
tgctggacgccatacatgag

SEQ ID NO: 419 - DNA, 20 bp, synthetic construct
  misc_feature 1..20: note = Synthetic
  source 1..20: mol_type = other DNA
agagtcagaaacaaggctgc

SEQ ID NO: 420 - DNA, 20 bp, synthetic construct
  misc_feature 1..20: note = Synthetic
  source 1..20: mol_type = other DNA
gcacagaagagtcagaaaca

SEQ ID NO: 421 - DNA, 20 bp, synthetic construct
  misc_feature 1..20: note = Synthetic
  source 1..20: mol_type = other DNA
ctgtgccttctgttgctcat

SEQ ID NO: 422 - DNA, 20 bp, synthetic construct
  misc_feature 1..20: note = Synthetic
  source 1..20: mol_type = other DNA
ccttctgttgctcattggat

SEQ ID NO: 423 - DNA, 20 bp, synthetic construct
  misc_feature 1..20: note = Synthetic
  source 1..20: mol_type = other DNA
cttctgttgctcattggatt

SEQ ID NO: 424 - DNA, 20 bp, synthetic construct
  misc_feature 1..20: note = Synthetic
  source 1..20: mol_type = other DNA
ccaatccaatgagcaacaga

SEQ ID NO: 425 - DNA, 20 bp, synthetic construct
  misc_feature 1..20: note = Synthetic
  source 1..20: mol_type = other DNA
gctcattggattgggagtct

SEQ ID NO: 426 - DNA, 20 bp, synthetic construct
  misc_feature 1..20: note = Synthetic
  source 1..20: mol_type = other DNA
atatagctcacgacataatt

SEQ ID NO: 427 - DNA, 20 bp, synthetic construct
  misc_feature 1..20: note = Synthetic
  source 1..20: mol_type = other DNA
aaggtaatcttgtattctct

SEQ ID NO: 428 - DNA, 20 bp, synthetic construct
  misc_feature 1..20: note = Synthetic
  source 1..20: mol_type = other DNA
caaggcttacatttgtgctc

SEQ ID NO: 429 - DNA, 20 bp, synthetic construct
  misc_feature 1..20: note = Synthetic
  source 1..20: mol_type = other DNA
acaaatgtaagccttgtcca

SEQ ID NO: 430 - DNA, 20 bp, synthetic construct
  misc_feature 1..20: note = Synthetic
  source 1..20: mol_type = other DNA
gtaagccttgtccaaggaga

SEQ ID NO: 431 - DNA, 20 bp, synthetic construct
  misc_feature 1..20: note = Synthetic
  source 1..20: mol_type = other DNA
cttgtccaaggagatggatt

SEQ ID NO: 432 - DNA, 20 bp, synthetic construct
  misc_feature 1..20: note = Synthetic
  source 1..20: mol_type = other DNA
caaatccatctccttggaca

SEQ ID NO: 433 - DNA, 20 bp, synthetic construct
  misc_feature 1..20: note = Synthetic
  source 1..20: mol_type = other DNA
aaggagatggatttggcata

SEQ ID NO: 434 - DNA, 20 bp, synthetic construct
  misc_feature 1..20: note = Synthetic
  source 1..20: mol_type = other DNA
ttatgccaaatccatctcct

SEQ ID NO: 435 - DNA, 20 bp, synthetic construct
  misc_feature 1..20: note = Synthetic
  source 1..20: mol_type = other DNA
taagtgatgatgtccaaaca

SEQ ID NO: 436 - DNA, 20 bp, synthetic construct
  misc_feature 1..20: note = Synthetic
  source 1..20: mol_type = other DNA
atgtttggacatcatcactt

SEQ ID NO: 437 - DNA, 20 bp, synthetic construct
  misc_feature 1..20: note = Synthetic
  source 1..20: mol_type = other DNA
tgatgatgtccaaacatggc

SEQ ID NO: 438 - DNA, 20 bp, synthetic construct
  misc_feature 1..20: note = Synthetic
  source 1..20: mol_type = other DNA
aacatggcaggagagtaaaa

SEQ ID NO: 439 - DNA, 20 bp, synthetic construct
  misc_feature 1..20: note = Synthetic
  source 1..20: mol_type = other DNA
ttttactctcctgccatgtt

SEQ ID NO: 440 - DNA, 20 bp, synthetic construct
  misc_feature 1..20: note = Synthetic
  source 1..20: mol_type = other DNA
gctggcattctgagcagcac

SEQ ID NO: 441 - DNA, 20 bp, synthetic construct
  misc_feature 1..20: note = Synthetic
  source 1..20: mol_type = other DNA
gttgtttatcttcaacaggc

SEQ ID NO: 442 - DNA, 20 bp, synthetic construct
  misc_feature 1..20: note = Synthetic
  source 1..20: mol_type = other DNA
ttttgttgtttatcttcaac

SEQ ID NO: 443 - DNA, 20 bp, synthetic construct
  misc_feature 1..20: note = Synthetic
  source 1..20: mol_type = other DNA
gataaacaacaaaaatgcat

SEQ ID NO: 444 - DNA, 20 bp, synthetic construct
  misc_feature 1..20: note = Synthetic
  source 1..20: mol_type = other DNA
aaaatgcattggtaaagccc

SEQ ID NO: 445 - DNA, 20 bp, synthetic construct
  misc_feature 1..20: note = Synthetic
  source 1..20: mol_type = other DNA
agactatctgtatttcctgt

SEQ ID NO: 446 - DNA, 20 bp, synthetic construct
  misc_feature 1..20: note = Synthetic
  source 1..20: mol_type = other DNA
gggattttataaattcctac

SEQ ID NO: 447 - DNA, 20 bp, synthetic construct
  misc_feature 1..20: note = Synthetic
  source 1..20: mol_type = other DNA
agagtagatcatatgactat

SEQ ID NO: 448 - DNA, 20 bp, synthetic construct
  misc_feature 1..20: note = Synthetic
  source 1..20: mol_type = other DNA
atagtcatatgatctactct

SEQ ID NO: 449 - DNA, 20 bp, synthetic construct
  misc_feature 1..20: note = Synthetic
  source 1..20: mol_type = other DNA
aatagtcatatgatctactc

SEQ ID NO: 450 - DNA, 20 bp, synthetic construct
  misc_feature 1..20: note = Synthetic
  source 1..20: mol_type = other DNA
tagatcatatgactattggc

SEQ ID NO: 451 - DNA, 20 bp, synthetic construct
  misc_feature 1..20: note = Synthetic
  source 1..20: mol_type = other DNA
agatcatatgactattggct

SEQ ID NO: 452 - DNA, 20 bp, synthetic construct
  misc_feature 1..20: note = Synthetic
  source 1..20: mol_type = other DNA
cctgaagaagattccactcg

SEQ ID NO: 453 - DNA, 20 bp, synthetic construct
  misc_feature 1..20: note = Synthetic
  source 1..20: mol_type = other DNA
ccacgagtggaatcttcttc

SEQ ID NO: 454 - DNA, 20 bp, synthetic construct
  misc_feature 1..20: note = Synthetic
  source 1..20: mol_type = other DNA
ttccactcgtggtatgagag

SEQ ID NO: 455 - DNA, 20 bp, synthetic construct
  misc_feature 1..20: note = Synthetic
  source 1..20: mol_type = other DNA
atccactctcataccacgag

SEQ ID NO: 456 - DNA, 20 bp, synthetic construct
  misc_feature 1..20: note = Synthetic
  source 1..20: mol_type = other DNA
aacaagaatagacacttacc

SEQ ID NO: 457 - DNA, 20 bp, synthetic construct
  misc_feature 1..20: note = Synthetic
  source 1..20: mol_type = other DNA
aattctttactttctctttc

SEQ ID NO: 458 - DNA, 20 bp, synthetic construct
  misc_feature 1..20: note = Synthetic
  source 1..20: mol_type = other DNA
attctttactttctctttca

SEQ ID NO: 459 - DNA, 20 bp, synthetic construct
  misc_feature 1..20: note = Synthetic
  source 1..20: mol_type = other DNA
gacttaaataacatgtattg

SEQ ID NO: 460 - DNA, 20 bp, synthetic construct
  misc_feature 1..20: note = Synthetic
  source 1..20: mol_type = other DNA
caatacatgttatttaagtc

SEQ ID NO: 461 - DNA, 20 bp, synthetic construct
  misc_feature 1..20: note = Synthetic
  source 1..20: mol_type = other DNA
aagaatgatatgtgagaaga

SEQ ID NO: 462 - DNA, 20 bp, synthetic construct
  misc_feature 1..20: note = Synthetic
  source 1..20: mol_type = other DNA
atggccaatccagtgcagct

SEQ ID NO: 463 - DNA, 20 bp, synthetic construct
  misc_feature 1..20: note = Synthetic
  source 1..20: mol_type = other DNA
agaaccaagctgcactggat

SEQ ID NO: 464 - DNA, 20 bp, synthetic construct
  misc_feature 1..20: note = Synthetic
  source 1..20: mol_type = other DNA
tatgtagaaccaagctgcac

SEQ ID NO: 465 - DNA, 20 bp, synthetic construct
  misc_feature 1..20: note = Synthetic
  source 1..20: mol_type = other DNA
agcttggttctacatatttt

SEQ ID NO: 466 - DNA, 20 bp, synthetic construct
  misc_feature 1..20: note = Synthetic
  source 1..20: mol_type = other DNA
gcttggttctacatatttta

SEQ ID NO: 467 - DNA, 20 bp, synthetic construct
  misc_feature 1..20: note = Synthetic
  source 1..20: mol_type = other DNA
tggttctacatattttaggg

SEQ ID NO: 468 - DNA, 20 bp, synthetic construct
  misc_feature 1..20: note = Synthetic
  source 1..20: mol_type = other DNA
acatattttagggaggcatg

SEQ ID NO: 469 - DNA, 20 bp, synthetic construct
  misc_feature 1..20: note = Synthetic
  source 1..20: mol_type = other DNA
tattttgaagatcagagatg

SEQ ID NO: 470 - DNA, 20 bp, synthetic construct
  misc_feature 1..20: note = Synthetic
  source 1..20: mol_type = other DNA
atcttcaaaatatcatgatt

SEQ ID NO: 471 - DNA, 20 bp, synthetic construct
  misc_feature 1..20: note = Synthetic
  source 1..20: mol_type = other DNA
tcttcaaaatatcatgattt

SEQ ID NO: 472 - DNA, 20 bp, synthetic construct
  misc_feature 1..20: note = Synthetic
  source 1..20: mol_type = other DNA
tatcatgatttgggttttcc

SEQ ID NO: 473 - DNA, 20 bp, synthetic construct
  misc_feature 1..20: note = Synthetic
  source 1..20: mol_type = other DNA
tctcttcttgttttacttcc

SEQ ID NO: 474 - DNA, 20 bp, synthetic construct
  misc_feature 1..20: note = Synthetic
  source 1..20: mol_type = other DNA
ctgtccacctctttgcatta

SEQ ID NO: 475 - DNA, 20 bp, synthetic construct
  misc_feature 1..20: note = Synthetic
  source 1..20: mol_type = other DNA
cttaatgcaaagaggtggac

SEQ ID NO: 476 - DNA, 20 bp, synthetic construct
  misc_feature 1..20: note = Synthetic
  source 1..20: mol_type = other DNA
cctctttgcattaaggtagt

SEQ ID NO: 477 - DNA, 20 bp, synthetic construct
  misc_feature 1..20: note = Synthetic
  source 1..20: mol_type = other DNA
actaccttaatgcaaagagg

SEQ ID NO: 478 - DNA, 20 bp, synthetic construct
  misc_feature 1..20: note = Synthetic
  source 1..20: mol_type = other DNA
ccaactaccttaatgcaaag

SEQ ID NO: 479 - DNA, 20 bp, synthetic construct
  misc_feature 1..20: note = Synthetic
  source 1..20: mol_type = other DNA
ttaaggtagttggtccacct

SEQ ID NO: 480 - DNA, 20 bp, synthetic construct
  misc_feature 1..20: note = Synthetic
  source 1..20: mol_type = other DNA
gaaggctggcatgaccaagg

SEQ ID NO: 481 - DNA, 20 bp, synthetic construct
  misc_feature 1..20: note = Synthetic
  source 1..20: mol_type = other DNA
gaggaaggctggcatgacca

SEQ ID NO: 482 - DNA, 20 bp, synthetic construct
  misc_feature 1..20: note = Synthetic
  source 1..20: mol_type = other DNA
ttaatgtgtgagaggaaggc

SEQ ID NO: 483 - DNA, 20 bp, synthetic construct
  misc_feature 1..20: note = Synthetic
  source 1..20: mol_type = other DNA
ttatttaatgtgtgagagga

SEQ ID NO: 484 - DNA, 20 bp, synthetic construct
  misc_feature 1..20: note = Synthetic
  source 1..20: mol_type = other DNA
aacattatttaatgtgtgag

SEQ ID NO: 485 - DNA, 20 bp, synthetic construct
  misc_feature 1..20: note = Synthetic
  source 1..20: mol_type = other DNA
ataatgtttgttctcacaga

SEQ ID NO: 486 - DNA, 20 bp, synthetic construct
  misc_feature 1..20: note = Synthetic
  source 1..20: mol_type = other DNA
tgtttgttctcacagaaggt

SEQ ID NO: 487 - DNA, 20 bp, synthetic construct
  misc_feature 1..20: note = Synthetic
  source 1..20: mol_type = other DNA
gtttgttctcacagaaggta

SEQ ID NO: 488 - DNA, 20 bp, synthetic construct
  misc_feature 1..20: note = Synthetic
  source 1..20: mol_type = other DNA
attgtgcttattttcataat

SEQ ID NO: 489 - DNA, 20 bp, synthetic construct
  misc_feature 1..20: note = Synthetic
  source 1..20: mol_type = other DNA
aattgtgcttattttcataa

SEQ ID NO: 490 - DNA, 20 bp, synthetic construct
  misc_feature 1..20: note = Synthetic
  source 1..20: mol_type = other DNA
gagtttttggaagatggaaa

SEQ ID NO: 491 - DNA, 20 bp, synthetic construct
  misc_feature 1..20: note = Synthetic
  source 1..20: mol_type = other DNA
aggtttgagtttttggaaga

SEQ ID NO: 492 - DNA, 20 bp, synthetic construct
  misc_feature 1..20: note = Synthetic
  source 1..20: mol_type = other DNA
ccaaaaactcaaacctgtga

SEQ ID NO: 493 - DNA, 20 bp, synthetic construct
  misc_feature 1..20: note = Synthetic
  source 1..20: mol_type = other DNA
caaaaactcaaacctgtgaa

SEQ ID NO: 494 - DNA, 20 bp, synthetic construct
  misc_feature 1..20: note = Synthetic
  source 1..20: mol_type = other DNA
ccttcacaggtttgagtttt

SEQ ID NO: 495 - DNA, 20 bp, synthetic construct
  misc_feature 1..20: note = Synthetic
  source 1..20: mol_type = other DNA
tattctaactttcccttcac

SEQ ID NO: 496 - DNA, 20 bp, synthetic construct
  misc_feature 1..20: note = Synthetic
  source 1..20: mol_type = other DNA
tcagaaaatatgtaagtcac

SEQ ID NO: 497 - DNA, 20 bp, synthetic construct
  misc_feature 1..20: note = Synthetic
  source 1..20: mol_type = other DNA
ctggaagtgggaggatggct

SEQ ID NO: 498 - DNA, 20 bp, synthetic construct
  misc_feature 1..20: note = Synthetic
  source 1..20: mol_type = other DNA
gaggtctggaagtgggagga

SEQ ID NO: 499 - DNA, 20 bp, synthetic construct
  misc_feature 1..20: note = Synthetic
  source 1..20: mol_type = other DNA
gaatgaggtctggaagtggg

SEQ ID NO: 500 - DNA, 20 bp, synthetic construct
  misc_feature 1..20: note = Synthetic
  source 1..20: mol_type = other DNA
gtcgaatgaggtctggaagt

SEQ ID NO: 501 - DNA, 20 bp, synthetic construct
  misc_feature 1..20: note = Synthetic
  source 1..20: mol_type = other DNA
agtcgaatgaggtctggaag

SEQ ID NO: 502 - DNA, 20 bp, synthetic construct
  misc_feature 1..20: note = Synthetic
  source 1..20: mol_type = other DNA
tccagacctcattcgacttc

SEQ ID NO: 503 - DNA, 20 bp, synthetic construct
  misc_feature 1..20: note = Synthetic
  source 1..20: mol_type = other DNA
gccagaagtcgaatgaggtc

SEQ ID NO: 504 - DNA, 20 bp, synthetic construct
  misc_feature 1..20: note = Synthetic
  source 1..20: mol_type = other DNA
atctcgccagaagtcgaatg

SEQ ID NO: 505 - DNA, 20 bp, synthetic construct
  misc_feature 1..20: note = Synthetic
  source 1..20: mol_type = other DNA
actcatttaattcgttgggt

SEQ ID NO: 506 - DNA, 20 bp, synthetic construct
  misc_feature 1..20: note = Synthetic
  source 1..20: mol_type = other DNA
ccaacgaattaaatgagtcc

SEQ ID NO: 507 - DNA, 20 bp, synthetic construct
  misc_feature 1..20: note = Synthetic
  source 1..20: mol_type = other DNA
caggactcatttaattcgtt

SEQ ID NO: 508 - DNA, 20 bp, synthetic construct
  misc_feature 1..20: note = Synthetic
  source 1..20: mol_type = other DNA
ccaggactcatttaattcgt

SEQ ID NO: 509 - DNA, 20 bp, synthetic construct
  misc_feature 1..20: note = Synthetic
  source 1..20: mol_type = other DNA
aataggagtacatcaaagcc

SEQ ID NO: 510 - DNA, 20 bp, synthetic construct
  misc_feature 1..20: note = Synthetic
  source 1..20: mol_type = other DNA
tgtttatttatgtttacaat

SEQ ID NO: 511-599 - skipped (89 entries)

SEQ ID NO: 600 - DNA, 1555 bp, synthetic construct
  misc_feature 1..1555: note = Synthetic
  source 1..1555: mol_type = other DNA
ggctcatttgcagacatatgggtgattggtacagtaggtttataaacagaagtttaaacttgtaagcttaagcttccgtttataaacagaagtttaaaattataggtcctgtttaacattcagctctgttaactcactcatctttttgtgtttttacactttgtcaagatttctttacatattcatcaatgtctgaagaagttacttatgcagatcttcaattccagaactccagtgagatggaaaaaatcccagaaattggcaaatttggggaaaaagcacctccagctccctctcatgtatggcgtccagcagccttgtttctgactcttctgtgccttctgttgctcattggattgggagtcttggcaagcatgtttcacgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggaatttataaaatcccagagtagatcatatgactattggctgggattatctcctgaagaagattccactcgtggtatgagagtggataatataatcaactcctctgcctgggttataagaaacgcacctgacttaaataacatgtattgtggatatataaatagactatatgttcaatattatcactgcacttataaaaaaagaatgatatgtgagaagatggccaatccagtgcagcttggttctacatattttagggaggcatgaggcatcaatcaaatacatttaaggagtgtagggggtgggggttctaggctataggtaaatttaaatattttctggttgacaattagttgagtttgtctgaagacctgggattttatcatgcagatgaaacatccaggtagcaagcttcagagagaatagactgtgaatgttaatgccagagaggtataatgaagcatgtcccacctcccactttccatcatggcctgaaccctggaggaagaggaagtccattcagatagttgtggggggccttcgaattttcattttcatttacgttcttccccttctggccaagatttgccagaggcaacatcaaaaaccagcaaattttaattttgtcccacagcgttgctagggtggcatggctccccatctcgggtccatcctatacttccatgggactccctatggctgaaggccttatgagtcaaaggacttatagccaattgattgttctaggccaggtaagaatggatatggacatgcatttattacctcttaaaattattattttaagtaaaagccaataaacaaaaacgaaaaggcaa

SEQ ID NO: 601 - DNA, 1428 bp, synthetic construct
  misc_feature 1..1428: note = Synthetic
  source 1..1428: mol_type = other DNA
ggaagaacagcctttcaaatttgacttctgcatgtggatagatttctttacatattcatcaatgtctgaagaagttacttatgcagatcttcaattccagaactccagtgagatggaaaaaatcccagaaattggcaaatttggggaaaaagcacctccagctccctctcatgtatggcgtccagcagccttgtttctgactcttctgtgccttctgttgctcattggattgggagtcttggcaagcatgtttcacgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggaatttataaaatcccagagtagatcatatgactattggctgggattatctcctgaagaagattccactcgtggtatgagagtggataatataatcaactcctctgcctgggttataagaaacgcacctgacttaaataacatgtattgtggatatataaatagactatatgttcaatattatcactgcacttataaaaaaagaatgatatgtgagaagatggccaatccagtgcagcttggttctacatattttagggaggcatgaggcatcaatcaaatacatttaaggagtgtagggggtgggggttctaggctataggtaaatttaaatattttctggttgacaattagttgagtttgtctgaagacctgggattttatcatgcagatgaaacatccaggtagcaagcttcagagagaatagactgtgaatgttaatgccagagaggtataatgaagcatgtcccacctcccactttccatcatggcctgaaccctggaggaagaggaagtccattcagatagttgtggggggccttcgaattttcattttcatttacgttcttccccttctggccaagatttgccagaggcaacatcaaaaaccagcaaattttaattttgtcccacagcgttgctagggtggcatggctccccatctcgggtccatcctatacttccatgggactccctatggctgaaggccttatgagtcaaaggacttatagccaattgattgttctaggccaggtaagaatggatatggacatgcatttattacctcttaaaattattattttaagtaaaagccaataaacaaaaacgaaaaggcaa

SEQ ID NO: 602 - DNA, 1476 bp, synthetic construct
  misc_feature 1..1476: note = Synthetic
  source 1..1476: mol_type = other DNA
ctatttagcattgctgctgccagccccaaccacatttctgattgcctaggaagaacagcctttcaaatttgacttctgcatgtggatagatttctttacatattcatcaatgtctgaagaagttacttatgcagatcttcaattccagaactccagtgagatggaaaaaatcccagaaattggcaaatttggggaaaaagcacctccagctccctctcatgtatggcgtccagcagccttgtttctgactcttctgtgccttctgttgctcattggattgggagtcttggcaagcatgtttcacgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggaatttataaaatcccagagtagatcatatgactattggctgggattatctcctgaagaagattccactcgtggtatgagagtggataatataatcaactcctctgcctgggttataagaaacgcacctgacttaaataacatgtattgtggatatataaatagactatatgttcaatattatcactgcacttataaaaaaagaatgatatgtgagaagatggccaatccagtgcagcttggttctacatattttagggaggcatgaggcatcaatcaaatacatttaaggagtgtagggggtgggggttctaggctataggtaaatttaaatattttctggttgacaattagttgagtttgtctgaagacctgggattttatcatgcagatgaaacatccaggtagcaagcttcagagagaatagactgtgaatgttaatgccagagaggtataatgaagcatgtcccacctcccactttccatcatggcctgaaccctggaggaagaggaagtccattcagatagttgtggggggccttcgaattttcattttcatttacgttcttccccttctggccaagatttgccagaggcaacatcaaaaaccagcaaattttaattttgtcccacagcgttgctagggtggcatggctccccatctcgggtccatcctatacttccatgggactccctatggctgaaggccttatgagtcaaaggacttatagccaattgattgttctaggccaggtaagaatggatatggacatgcatttattacctcttaaaattattattttaagtaaaagccaataaacaaaaacgaaaaggcaa

SEQ ID NO: 603 - DNA, 1553 bp, synthetic construct
  misc_feature 1..1553: note = Synthetic
  source 1..1553: mol_type = other DNA
ggctcatttgcagacatatgggtgattggtacagtaggtttataaacagaagtttaaacttgtaagcttaagcttccgtttataaacagaagtttaaaattataggtcctgtttaacattcagctctgttaactcactcatctttttgtgtttttacactttgtcaagatttctttacatattcatcaatgtctgaagaagttacttatgcagatcttcaattccagaactccagtgagatggaaaaaatcccagaaattggcaaatttggggaaaaagcacctccagctccctctcatgtatggcgtccagcagccttgtttctgactcttctgtgccttctgttgctcattggattgggagtcttggcaagcatgtttcacgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggaatttataaaatcccagagtagatcatatgactattggctgggattatctcctgaagaagattccactcgtggtatgagagtggataatataatcaactcctctgcctgaaaatatcaaacgaagaaagaaaccagagtctcaacctgctggacactattggaagtccatcatttaacacgtttttagtatatacttttagcaggagacagctctgagtcaactgtgttgaggtgccaccacagcgagtttaggcactcagatccctgcatactcatcacattgggccataatggcaaatagaattttttgttttgttttgtttgtttgctttttctttcacatagaaatagtaagtgtaggagtgtgggtcagaaagaaaaggtggccctacctctgatggttggcaatgataggatacaatgggagataagctatctacaaatggagtggagaaggatatatatttcaaaggcctaatttgtagtgaaagactagagacaaaggtaatgtgtgtgtcaggagagagtacagatggaatcttgttttgcaaacgtagaatatgtatgtgtttgtaattattgcaaatggaatggtaatctataatggaatggaaaacattgtagatattttcagttatcaaaaagaaaactgaaaaagtatataataattgtatgtatgatatatatatgtgtgtgtgtgtgtatatatatcttcactttataactctgtgttgttttggggtttgtttctgaaagggggttgtaataaatgacatctgtactatgtcaccacaaataaatctcattcttaaacatttaattgatgaactta

SEQ ID NO: 604 - DNA, 1456 bp, synthetic construct
  misc_feature 1..1456: note = Synthetic
  source 1..1456: mol_type = other DNA
ggctcatttgcagacatatgggtgattggtacagtaggtttataaacagaagtttaaacttgtaagcttaagcttccgtttataaacagaagtttaaaattataggtcctgtttaacattcagctctgttaactcactcatctttttgtgtttttacactttgtcaagatttctttacatattcatcaatgtctgaagaagttacttatgcagatcttcaattccagaactccagtgagatggaaaaaatcccagaaattggcaaatttggggaaaaagttcacgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggaatttataaaatcccagagtagatcatatgactattggctgggattatctcctgaagaagattccactcgtggtatgagagtggataatataatcaactcctctgcctgggttataagaaacgcacctgacttaaataacatgtattgtggatatataaatagactatatgttcaatattatcactgcacttataaaaaaagaatgatatgtgagaagatggccaatccagtgcagcttggttctacatattttagggaggcatgaggcatcaatcaaatacatttaaggagtgtagggggtgggggttctaggctataggtaaatttaaatattttctggttgacaattagttgagtttgtctgaagacctgggattttatcatgcagatgaaacatccaggtagcaagcttcagagagaatagactgtgaatgttaatgccagagaggtataatgaagcatgtcccacctcccactttccatcatggcctgaaccctggaggaagaggaagtccattcagatagttgtggggggccttcgaattttcattttcatttacgttcttccccttctggccaagatttgccagaggcaacatcaaaaaccagcaaattttaattttgtcccacagcgttgctagggtggcatggctccccatctcgggtccatcctatacttccatgggactccctatggctgaaggccttatgagtcaaaggacttatagccaattgattgttctaggccaggtaagaatggatatggacatgcatttattacctcttaaaattattattttaagtaaaagccaataaacaaaaacgaaaaggcaa

SEQ ID NO: 605-699 - skipped (95 entries)

SEQ ID NO: 700 - DNA, 1479 bp, synthetic construct
  misc_feature 1..1479: note = Synthetic
  source 1..1479: mol_type = other DNA
gtcaggttcatggttacgaagctgctgaccccaggatcccagcccgtgggagagaagggggtctctgacagcccccacccctccccactgccagatccttattgggtctgagtttcaggggtggggccccagctggaggttataaaacagctcaatcggggagtacaaccttcggtttctcttcggggaaagctgctttcagcgcacacgggaagatatcagaaacatcctaggatcaggacaccccagatcttctcaactggaaccacgaaggctgtttcttccacacagtactttgatctccatttaagcaggcacctctgtcctgcgttccggagctgcgttcccgatggtcctcctttggctcacgctgctcctgatcgccctgccctgtctcctgcaaacgaaggaaggtgggaagccttgggcaggtgcggagaatctgacctgctggattcatgacgtggatttcttgagctgcagctgggcggtaggcccgggggcccccgcggacgtccagtacgacctgtacttgaacgttgccaacaggcgtcaacagtacgagtgtcttcactacaaaacggatgctcagggaacacgtatcgggtgtcgtttcgatgacatctctcgactctccagcggttctcaaagttcccacatcctggtgcggggcaggagcgcagccttcggtatcccctgcacagataagtttgtcgtcttttcacagattgagatattaactccacccaacatgactgcaaagtgtaataagacacattcctttatgcactggaaaatgagaagtcatttcaatcgcaaatttcgctatgagcttcagatacaaaagagaatgcagcctgtaatcacagaacaggtcagagacagaacctccttccagctactcaatcctggaacgtacacagtacaaataagagcccgggaaagagtgtatgaattcttgagcgcctggagcaccccccagcgcttcgagtgcgaccaggaggagggcgcaaacacacgtgcctggcggacgtcgctgctgatcgcgctggggacgctgctggccctggtctgtgtcttcgtgatctgcagaaggtatctggtgatgcagagactctttccccgcatccctcacatgaaagaccccatcggtgacagcttccaaaacgacaagctggtggtctgggaggcgggcaaagccggcctggaggagtgtctggtgactgaagtacaggtcgtgcagaaaacttgagactggggttcagggcttgtgggggtctgcctcaatctccctggccgggccaggcgcctgcacagactggctgctggacctgcgcacgcagcccaggaatggacattcctaacgggtggtgggcatgggagatgcctgtgtaatttcgtccgaagctgccaggaagaagaacagaactttgtgtgtttatttcatgataaagtgatttttttttttttaacccaaaa

SEQ ID NO: 701 - DNA, 1541 bp, synthetic construct
  misc_feature 1..1541: note = Synthetic
  source 1..1541: mol_type = other DNA
cttcggtttctcttcggggaaagctgctttcagcgcacacgggaagatatcagaaacatcctaggatcaggacaccccagatcttctcaactggaaccacgaaggctgtttcttccacacagtactttgatctccatttaagcaggcacctctgtcctgcgttccggagctgcgttcccgatggtcctcctttggctcacgctgctcctgatcgccctgccctgtctcctgcaaacgaaggaagatccaaacccaccaatcacgaacctaaggatgaaagcaaaggctcagcagttgacctgggaccttaacagaaatgtgaccgatatcgagtgtgttaaagacgccgactattctatgccggcagtgaacaatagctattgccagtttggagcaatttccttatgtgaagtgaccaactacaccgtccgagtggccaacccaccattctccacgtggatcctcttccctgagaacagtgggaagccttgggcaggtgcggagaatctgacctgctggattcatgacgtggatttcttgagctgcagctgggcggtaggcccgggggcccccgcggacgtccagtacgacctgtacttgaacgttgccaacaggcgtcaacagtacgagtgtcttcactacaaaacggatgctcagggaacacgtatcgggtgtcgtttcgatgacatctctcgactctccagcggttctcaaagttcccacatcctggtgcggggcaggagcgcagccttcggtatcccctgcacagataagtttgtcgtcttttcacagattgagatattaactccacccaacatgactgcaaagtgtaataagacacattcctttatgcactggaaaatgagaagtcatttcaatcgcaaatttcgctatgagcttcagatacaaaagagaatgcagcctgtaatcacagaacaggtcagagacagaacctccttccagctactcaatcctggaacgtacacagtacaaataagagcccgggaaagagtgtatgaattcttgagcgcctggagcaccccccagcgcttcgagtgcgaccaggaggagggcgcaaacacacgtgcctggcggacgtcgctgctgatcgcgctggggacgctgctggccctggtctgtgtcttcgtgatctgcagaaggtatctggtgatgcagagactctttccccgcatccctcacatgaaagaccccatcggtgacagcttccaaaacgacaagctggtggtctgggaggcgggcaaagccggcctggaggagtgtctggtgactgaagtacaggtcgtgcagaaaacttgagactggggttcagggcttgtgggggtctgcctcaatctccctggccgggccaggcgcctgcacagactggctgctggacctgcgcacgcagcccaggaatggacattcctaacgggtggtgggcatgggagatgcctgtgtaatttcgtccgaagctgccaggaagaagaacagaactttgtgtgtttatttcatgataaagtgatttttttttttttaaccca

SEQ ID NO: 702-800 - skipped (99 entries)

SEQ ID NO: 801 - DNA, 20 bp, synthetic construct
  misc_feature 1..20: note = Synthetic
  source 1..20: mol_type = other DNA
gccctgtctcctgcaaacga

SEQ ID NO: 802 - DNA, 20 bp, synthetic construct
  misc_feature 1..20: note = Synthetic
  source 1..20: mol_type = other DNA
tgagccaaaggaggaccatc

SEQ ID NO: 803 - DNA, 20 bp, synthetic construct
  misc_feature 1..20: note = Synthetic
  source 1..20: mol_type = other DNA
tcaggagcagcgtgagccaa

SEQ ID NO: 804 - DNA, 20 bp, synthetic construct
  misc_feature 1..20: note = Synthetic
  source 1..20: mol_type = other DNA
tccttcgtttgcaggagaca

SEQ ID NO: 805 - DNA, 20 bp, synthetic construct
  misc_feature 1..20: note = Synthetic
  source 1..20: mol_type = other DNA
atccacgtcatgaatccagc

SEQ ID NO: 806 - DNA, 20 bp, synthetic construct
  misc_feature 1..20: note = Synthetic
  source 1..20: mol_type = other DNA
caggtcgtactggacgtccg

SEQ ID NO: 807 - DNA, 20 bp, synthetic construct
  misc_feature 1..20: note = Synthetic
  source 1..20: mol_type = other DNA
tttcttgagctgcagctggg

SEQ ID NO: 808 - DNA, 20 bp, synthetic construct
  misc_feature 1..20: note = Synthetic
  source 1..20: mol_type = other DNA
ggtcgtactggacgtccgcg

SEQ ID NO: 809 - DNA, 20 bp, synthetic construct
  misc_feature 1..20: note = Synthetic
  source 1..20: mol_type = other DNA
agttcccacatcctggtgcg

SEQ ID NO: 810 - DNA, 20 bp, synthetic construct
  misc_feature 1..20: note = Synthetic
  source 1..20: mol_type = other DNA
cactacaaaacggatgctca

SEQ ID NO: 811 - DNA, 20 bp, synthetic construct
  misc_feature 1..20: note = Synthetic
  source 1..20: mol_type = other DNA
tcgtttgcaggagacagggc

SEQ ID NO: 812 - DNA, 20 bp, synthetic construct
  misc_feature 1..20: note = Synthetic
  source 1..20: mol_type = other DNA
gatggtcctcctttggctca

SEQ ID NO: 813 - DNA, 20 bp, synthetic construct
  misc_feature 1..20: note = Synthetic
  source 1..20: mol_type = other DNA
ttggctcacgctgctcctga

SEQ ID NO: 814 - DNA, 20 bp, synthetic construct
  misc_feature 1..20: note = Synthetic
  source 1..20: mol_type = other DNA
tgtctcctgcaaacgaagga

SEQ ID NO: 815 - DNA, 20 bp, synthetic construct
  misc_feature 1..20: note = Synthetic
  source 1..20: mol_type = other DNA
gctggattcatgacgtggat

SEQ ID NO: 816 - DNA, 20 bp, synthetic construct
  misc_feature 1..20: note = Synthetic
  source 1..20: mol_type = other DNA
cggacgtccagtacgacctg

SEQ ID NO: 817 - DNA, 20 bp, synthetic construct
  misc_feature 1..20: note = Synthetic
  source 1..20: mol_type = other DNA
cccagctgcagctcaagaaa

SEQ ID NO: 818 - DNA, 20 bp, synthetic construct
  misc_feature 1..20: note = Synthetic
  source 1..20: mol_type = other DNA
cgcggacgtccagtacgacc

SEQ ID NO: 819 - DNA, 20 bp, synthetic construct
  misc_feature 1..20: note = Synthetic
  source 1..20: mol_type = other DNA
cgcaccaggatgtgggaact

SEQ ID NO: 820 - DNA, 20 bp, synthetic construct
  misc_feature 1..20: note = Synthetic
  source 1..20: mol_type = other DNA
tgagcatccgttttgtagtg

SEQ ID NO: 821 - RNA, 20 nt, synthetic construct
  misc_feature 1..20: note = Synthetic
  source 1..20: mol_type = other RNA
gccctgtctcctgcaaacga

SEQ ID NO: 822 - RNA, 20 nt, synthetic construct
  misc_feature 1..20: note = Synthetic
  source 1..20: mol_type = other RNA
tgagccaaaggaggaccatc

SEQ ID NO: 823 - RNA, 20 nt, synthetic construct
  misc_feature 1..20: note = Synthetic
  source 1..20: mol_type = other RNA
tcaggagcagcgtgagccaa

SEQ ID NO: 824 - RNA, 20 nt, synthetic construct
  misc_feature 1..20: note = Synthetic
  source 1..20: mol_type = other RNA
tccttcgtttgcaggagaca

SEQ ID NO: 825 - RNA, 20 nt, synthetic construct
  misc_feature 1..20: note = Synthetic
  source 1..20: mol_type = other RNA
gctggattcatgacgtggat

SEQ ID NO: 826 - RNA, 20 nt, synthetic construct
  misc_feature 1..20: note = Synthetic
  source 1..20: mol_type = other RNA
caggtcgtactggacgtccg

SEQ ID NO: 827 - RNA, 20 nt, synthetic construct
  misc_feature 1..20: note = Synthetic
  source 1..20: mol_type = other RNA
tttcttgagctgcagctggg

SEQ ID NO: 828 - RNA, 20 nt, synthetic construct
  misc_feature 1..20: note = Synthetic
  source 1..20: mol_type = other RNA
ggtcgtactggacgtccgcg

SEQ ID NO: 829 - RNA, 20 nt, synthetic construct
  misc_feature 1..20: note = Synthetic
  source 1..20: mol_type = other RNA
agttcccacatcctggtgcg

SEQ ID NO: 830 - RNA, 20 nt, synthetic construct
  misc_feature 1..20: note = Synthetic
  source 1..20: mol_type = other RNA
tgagcatccgttttgtagtg

SEQ ID NO: 831-839 - skipped (9 entries)

SEQ ID NO: 840 - DNA, 20 bp, synthetic construct
  misc_feature 1..20: note = Synthetic
  source 1..20: mol_type = other DNA
ttcatccttaggttcgtgat

SEQ ID NO: 841 - DNA, 20 bp, synthetic construct
  misc_feature 1..20: note = Synthetic
  source 1..20: mol_type = other DNA
atcacgaacctaaggatgaa

SEQ ID NO: 842 - DNA, 20 bp, synthetic construct
  misc_feature 1..20: note = Synthetic
  source 1..20: mol_type = other DNA
ttgacgcctgctgcggtaag

SEQ ID NO: 843 - DNA, 20 bp, synthetic construct
  misc_feature 1..20: note = Synthetic
  source 1..20: mol_type = other DNA
cttaccgcagcaggcgtcaa

SEQ ID NO: 844 - DNA, 20 bp, synthetic construct
  misc_feature 1..20: note = Synthetic
  source 1..20: mol_type = other DNA
cgagtgtcttcactacaaaa

SEQ ID NO: 845 - DNA, 20 bp, synthetic construct
  misc_feature 1..20: note = Synthetic
  source 1..20: mol_type = other DNA
ttttgtagtgaagacactcg

SEQ ID NO: 846 - DNA, 20 bp, synthetic construct
  misc_feature 1..20: note = Synthetic
  source 1..20: mol_type = other DNA
atgctcagggaacacgtatc

SEQ ID NO: 847 - DNA, 20 bp, synthetic construct
  misc_feature 1..20: note = Synthetic
  source 1..20: mol_type = other DNA
gatacgtgttccctgagcat

SEQ ID NO: 848 - RNA, 20 nt, synthetic construct
  misc_feature 1..20: note = Synthetic
  source 1..20: mol_type = other RNA
ttcatccttaggttcgtgat

SEQ ID NO: 849 - RNA, 20 nt, synthetic construct
  misc_feature 1..20: note = Synthetic
  source 1..20: mol_type = other RNA
ttgacgcctgctgcggtaag

SEQ ID NO: 850 - RNA, 20 nt, synthetic construct
  misc_feature 1..20: note = Synthetic
  source 1..20: mol_type = other RNA
cgagtgtcttcactacaaaa

SEQ ID NO: 851 - RNA, 20 nt, synthetic construct
  misc_feature 1..20: note = Synthetic
  source 1..20: mol_type = other RNA
atgctcagggaacacgtatc

SEQ ID NO: 852-899 - skipped (48 entries)

SEQ ID NO: 900 - DNA, 20 bp, synthetic construct
  misc_feature 1..20: note = Synthetic
  source 1..20: mol_type = other DNA
ttccggagctgcgttcccga

SEQ ID NO: 901 - DNA, 20 bp, synthetic construct
  misc_feature 1..20: note = Synthetic
  source 1..20: mol_type = other DNA
gaccatcgggaacgcagctc

SEQ ID NO: 902 - DNA, 20 bp, synthetic construct
  misc_feature 1..20: note = Synthetic
  source 1..20: mol_type = other DNA
cgttcccgatggtcctcctt

SEQ ID NO: 903 - DNA, 20 bp, synthetic construct
  misc_feature 1..20: note = Synthetic
  source 1..20: mol_type = other DNA
gtgagccaaaggaggaccat

SEQ ID NO: 904 - DNA, 20 bp, synthetic construct
  misc_feature 1..20: note = Synthetic
  source 1..20: mol_type = other DNA
ggagcagcgtgagccaaagg

SEQ ID NO: 905 - DNA, 20 bp, synthetic construct
  misc_feature 1..20: note = Synthetic
  source 1..20: mol_type = other DNA
ggagacagggcagggcgatc

SEQ ID NO: 906 - DNA, 20 bp, synthetic construct
  misc_feature 1..20: note = Synthetic
  source 1..20: mol_type = other DNA
cgtttgcaggagacagggca

SEQ ID NO: 907 - DNA, 20 bp, synthetic construct
  misc_feature 1..20: note = Synthetic
  source 1..20: mol_type = other DNA
tcgtttgcaggagacagggc

SEQ ID NO: 908 - DNA, 20 bp, synthetic construct
  misc_feature 1..20: note = Synthetic
  source 1..20: mol_type = other DNA
tgtctcctgcaaacgaagga

SEQ ID NO: 909 - DNA, 20 bp, synthetic construct
  misc_feature 1..20: note = Synthetic
  source 1..20: mol_type = other DNA
ttccttcgtttgcaggagac

SEQ ID NO: 910 - DNA, 20 bp, synthetic construct
  misc_feature 1..20: note = Synthetic
  source 1..20: mol_type = other DNA
tcttaccttccttcgtttgc

SEQ ID NO: 911 - DNA, 20 bp, synthetic construct
  misc_feature 1..20: note = Synthetic
  source 1..20: mol_type = other DNA
aaacgaaggaaggtaagaac

SEQ ID NO: 912 - DNA, 20 bp, synthetic construct
  misc_feature 1..20: note = Synthetic
  source 1..20: mol_type = other DNA
gatctaaaacggtgacaggt

SEQ ID NO: 913 - DNA, 20 bp, synthetic construct
  misc_feature 1..20: note = Synthetic
  source 1..20: mol_type = other DNA
tttggatctaaaacggtgac

SEQ ID NO: 914 - DNA, 20 bp, synthetic construct
  misc_feature 1..20: note = Synthetic
  source 1..20: mol_type = other DNA
tggtgggtttggatctaaaa

SEQ ID NO: 915 - DNA, 20 bp, synthetic construct
  misc_feature 1..20: note = Synthetic
  source 1..20: mol_type = other DNA
aggttcgtgattggtgggtt

SEQ ID NO: 916 - DNA, 20 bp, synthetic construct
  misc_feature 1..20: note = Synthetic
  source 1..20: mol_type = other DNA
acccaccaatcacgaaccta

SEQ ID NO: 917 - DNA, 20 bp, synthetic construct
  misc_feature 1..20: note = Synthetic
  source 1..20: mol_type = other DNA
tccttaggttcgtgattggt

SEQ ID NO: 918 - DNA, 20 bp, synthetic construct
  misc_feature 1..20: note = Synthetic
  source 1..20: mol_type = other DNA
atccttaggttcgtgattgg

SEQ ID NO: 919 - DNA, 20 bp, synthetic construct
  misc_feature 1..20: note = Synthetic
  source 1..20: mol_type = other DNA
gaacctaaggatgaaagcaa

SEQ ID NO: 920 - DNA, 20 bp, synthetic construct
  misc_feature 1..20: note = Synthetic
  source 1..20: mol_type = other DNA
gagcctttgctttcatcctt

SEQ ID NO: 921 - DNA, 20 bp, synthetic construct
  misc_feature 1..20: note = Synthetic
  source 1..20: mol_type = other DNA
caaaggctcagcagttgacc

SEQ ID NO: 922 - DNA, 20 bp, synthetic construct
  misc_feature 1..20: note = Synthetic
  source 1..20: mol_type = other DNA
aaaggctcagcagttgacct

SEQ ID NO: 923 - DNA, 20 bp, synthetic construct
  misc_feature 1..20: note = Synthetic
  source 1..20: mol_type = other DNA
cacatttctgttaaggtccc

SEQ ID NO: 924 - DNA, 20 bp, synthetic construct
  misc_feature 1..20: note = Synthetic
  source 1..20: mol_type = other DNA
tatcggtcacatttctgtta

SEQ ID NO: 925 - DNA, 20 bp, synthetic construct
  misc_feature 1..20: note = Synthetic
  source 1..20: mol_type = other DNA
gtctttaacacactcgatat

SEQ ID NO: 926 - DNA, 20 bp, synthetic construct
  misc_feature 1..20: note = Synthetic
  source 1..20: mol_type = other DNA
agacgccgactattctatgc

SEQ ID NO: 927 - DNA, 20 bp, synthetic construct
  misc_feature 1..20: note = Synthetic
  source 1..20: mol_type = other DNA
atttaccggcatagaatagt

SEQ ID NO: 928 - DNA, 20 bp, synthetic construct
  misc_feature 1..20: note = Synthetic
  source 1..20: mol_type = other DNA
caatagagagtatgatttac

SEQ ID NO: 929 - DNA, 20 bp, synthetic construct
  misc_feature 1..20: note = Synthetic
  source 1..20: mol_type = other DNA
catagtcctatgtctctctt

SEQ ID NO: 930 - DNA, 20 bp, synthetic construct
  misc_feature 1..20: note = Synthetic
  source 1..20: mol_type = other DNA
tcactgcctaagagagacat

SEQ ID NO: 931 - DNA, 20 bp, synthetic construct
  misc_feature 1..20: note = Synthetic
  source 1..20: mol_type = other DNA
aacaatagctattgccagtt

SEQ ID NO: 932 - DNA, 20 bp, synthetic construct
  misc_feature 1..20: note = Synthetic
  source 1..20: mol_type = other DNA
ataaggaaattgctccaaac

SEQ ID NO: 933 - DNA, 20 bp, synthetic construct
  misc_feature 1..20: note = Synthetic
  source 1..20: mol_type = other DNA
gtagttggtcacttcacata

SEQ ID NO: 934 - DNA, 20 bp, synthetic construct
  misc_feature 1..20: note = Synthetic
  source 1..20: mol_type = other DNA
gaccaactacaccgtccgag

SEQ ID NO: 935 - DNA, 20 bp, synthetic construct
  misc_feature 1..20: note = Synthetic
  source 1..20: mol_type = other DNA
ggccactcggacggtgtagt

SEQ ID NO: 936 - DNA, 20 bp, synthetic construct
  misc_feature 1..20: note = Synthetic
  source 1..20: mol_type = other DNA
tggtgggttggccactcgga

SEQ ID NO: 937 - DNA, 20 bp, synthetic construct
  misc_feature 1..20: note = Synthetic
  source 1..20: mol_type = other DNA
agaatggtgggttggccact

SEQ ID NO: 938 - DNA, 20 bp, synthetic construct
  misc_feature 1..20: note = Synthetic
  source 1..20: mol_type = other DNA
ccaacccaccattctccacg

SEQ ID NO: 939 - DNA, 20 bp, synthetic construct
  misc_feature 1..20: note = Synthetic
  source 1..20: mol_type = other DNA
ccacgtggagaatggtgggt

SEQ ID NO: 940 - DNA, 20 bp, synthetic construct
  misc_feature 1..20: note = Synthetic
  source 1..20: mol_type = other DNA
ggatccacgtggagaatggt

SEQ ID NO: 941 - DNA, 20 bp, synthetic construct
  misc_feature 1..20: note = Synthetic
  source 1..20: mol_type = other DNA
aggatccacgtggagaatgg

SEQ ID NO: 942 - DNA, 20 bp, synthetic construct
  misc_feature 1..20: note = Synthetic
  source 1..20: mol_type = other DNA
aagaggatccacgtggagaa

SEQ ID NO: 943 - DNA, 20 bp, synthetic construct
  misc_feature 1..20: note = Synthetic
  source 1..20: mol_type = other DNA
ctcagggaagaggatccacg

SEQ ID NO: 944 - DNA, 20 bp, synthetic construct
  misc_feature 1..20: note = Synthetic
  source 1..20: mol_type = other DNA
tctcactgttctcagggaag

SEQ ID NO: 945 - DNA, 20 bp, synthetic construct
  misc_feature 1..20: note = Synthetic
  source 1..20: mol_type = other DNA
catttttctcactgttctca

SEQ ID NO: 946 - DNA, 20 bp, synthetic construct
  misc_feature 1..20: note = Synthetic
  source 1..20: mol_type = other DNA
acatttttctcactgttctc

SEQ ID NO: 947 - DNA, 20 bp, synthetic construct
  misc_feature 1..20: note = Synthetic
  source 1..20: mol_type = other DNA
tctttcatgtttgtgaaccc

SEQ ID NO: 948 - DNA, 20 bp, synthetic construct
  misc_feature 1..20: note = Synthetic
  source 1..20: mol_type = other DNA
ttcatgtttgtgaacccagg

SEQ ID NO: 949 - DNA, 20 bp, synthetic construct
  misc_feature 1..20: note = Synthetic
  source 1..20: mol_type = other DNA
tcatgtttgtgaacccaggt

SEQ ID NO: 950 - DNA, 20 bp, synthetic construct
  misc_feature 1..20: note = Synthetic
  source 1..20: mol_type = other DNA
tgaacccaggtgggaagcct

SEQ ID NO: 951 - DNA, 20 bp, synthetic construct
  misc_feature 1..20: note = Synthetic
  source 1..20: mol_type = other DNA
gaacccaggtgggaagcctt

SEQ ID NO: 952 - DNA, 20 bp, synthetic construct
  misc_feature 1..20: note = Synthetic
  source 1..20: mol_type = other DNA
ccaggtgggaagccttgggc

SEQ ID NO: 953 - DNA, 20 bp, synthetic construct
  misc_feature 1..20: note = Synthetic
  source 1..20: mol_type = other DNA
ctgcccaaggcttcccacct

SEQ ID NO: 954 - DNA, 20 bp, synthetic construct
  misc_feature 1..20: note = Synthetic
  source 1..20: mol_type = other DNA
cctgcccaaggcttcccacc

SEQ ID NO: 955 - DNA, 20 bp, synthetic construct
  misc_feature 1..20: note = Synthetic
  source 1..20: mol_type = other DNA
tgggaagccttgggcaggtg

SEQ ID NO: 956 - DNA, 20 bp, synthetic construct
  misc_feature 1..20: note = Synthetic
  source 1..20: mol_type = other DNA
agattctccgcacctgccca

SEQ ID NO: 957 - DNA, 20 bp, synthetic construct
  misc_feature 1..20: note = Synthetic
  source 1..20: mol_type = other DNA
gtgcggagaatctgacctgc

SEQ ID NO: 958 - DNA, 20 bp, synthetic construct
  misc_feature 1..20: note = Synthetic
  source 1..20: mol_type = other DNA
gacctgctggattcatgacg

SEQ ID NO: 959 - DNA, 20 bp, synthetic construct
  misc_feature 1..20: note = Synthetic
  source 1..20: mol_type = other DNA
tggatttcttgagctgcagc

SEQ ID NO: 960 - DNA, 20 bp, synthetic construct
  misc_feature 1..20: note = Synthetic
  source 1..20: mol_type = other DNA
ggatttcttgagctgcagct

SEQ ID NO: 961 - DNA, 20 bp, synthetic construct
  misc_feature 1..20: note = Synthetic
  source 1..20: mol_type = other DNA
ttgagctgcagctgggcggt

SEQ ID NO: 962 - DNA, 20 bp, synthetic construct
  misc_feature 1..20: note = Synthetic
  source 1..20: mol_type = other DNA
ctgcagctgggcggtaggcc

SEQ ID NO: 963 - DNA, 20 bp, synthetic construct
  misc_feature 1..20: note = Synthetic
  source 1..20: mol_type = other DNA
tgcagctgggcggtaggccc

SEQ ID NO: 964 - DNA, 20 bp, synthetic construct
  misc_feature 1..20: note = Synthetic
  source 1..20: mol_type = other DNA
gcagctgggcggtaggcccg

SEQ ID NO: 965 - DNA, 20 bp, synthetic construct
  misc_feature 1..20: note = Synthetic
  source 1..20: mol_type = other DNA
cagctgggcggtaggcccgg

SEQ ID NO: 966 - DNA, 20 bp, synthetic construct
  misc_feature 1..20: note = Synthetic
  source 1..20: mol_type = other DNA
ggtaggcccgggggcccccg

SEQ ID NO: 967 - DNA, 20 bp, synthetic construct
  misc_feature 1..20: note = Synthetic
  source 1..20: mol_type = other DNA
ggacgtccgcgggggccccc

SEQ ID NO: 968 - DNA, 20 bp, synthetic construct
  misc_feature 1..20: note = Synthetic
  source 1..20: mol_type = other DNA
tggacgtccgcgggggcccc

SEQ ID NO: 969 - DNA, 20 bp, synthetic construct
  misc_feature 1..20: note = Synthetic
  source 1..20: mol_type = other DNA
gtcgtactggacgtccgcgg

SEQ ID NO: 970 - DNA, 20 bp, synthetic construct
  misc_feature 1..20: note = Synthetic
  source 1..20: mol_type = other DNA
aggtcgtactggacgtccgc

SEQ ID NO: 971 - DNA, 20 bp, synthetic construct
  misc_feature 1..20: note = Synthetic
  source 1..20: mol_type = other DNA
cgttcaagtacaggtcgtac

SEQ ID NO: 972 - DNA, 20 bp, synthetic construct
  misc_feature 1..20: note = Synthetic
  source 1..20: mol_type = other DNA
gtacttgaacgttgccaagt

SEQ ID NO: 973 - DNA, 20 bp, synthetic construct
  misc_feature 1..20: note = Synthetic
  source 1..20: mol_type = other DNA
acttggcaacgttcaagtac

SEQ ID NO: 974 - DNA, 20 bp, synthetic construct
  misc_feature 1..20: note = Synthetic
  source 1..20: mol_type = other DNA
ttgccaagtaggtgtgcccg

SEQ ID NO: 975 - DNA, 20 bp, synthetic construct
  misc_feature 1..20: note = Synthetic
  source 1..20: mol_type = other DNA
tgccaagtaggtgtgcccgt

SEQ ID NO: 976 - DNA, 20 bp, synthetic construct
  misc_feature 1..20: note = Synthetic
  source 1..20: mol_type = other DNA
tgcccacgggcacacctact

SEQ ID NO: 977 - DNA, 20 bp, synthetic construct
  misc_feature 1..20: note = Synthetic
  source 1..20: mol_type = other DNA
accttaccgcttaccgcagc

SEQ ID NO: 978 - DNA, 20 bp, synthetic construct
  misc_feature 1..20: note = Synthetic
  source 1..20: mol_type = other DNA
gctgcggtaagcggtaaggt

SEQ ID NO: 979 - DNA, 20 bp, synthetic construct
  misc_feature 1..20: note = Synthetic
  source 1..20: mol_type = other DNA
gcctgctgcggtaagcggta

SEQ ID NO: 980 - DNA, 20 bp, synthetic construct
  misc_feature 1..20: note = Synthetic
  source 1..20: mol_type = other DNA
cgtactgttgacgcctgctg

SEQ ID NO: 981 - DNA, 20 bp, synthetic construct
  misc_feature 1..20: note = Synthetic
  source 1..20: mol_type = other DNA
tcactacaaaacggatgctc

SEQ ID NO: 982 - DNA, 20 bp, synthetic construct
  misc_feature 1..20: note = Synthetic
  source 1..20: mol_type = other DNA
gatgctcagggaacacgtat

SEQ ID NO: 983 - DNA, 20 bp, synthetic construct
  misc_feature 1..20: note = Synthetic
  source 1..20: mol_type = other DNA
gacatctctcgactctccag

SEQ ID NO: 984 - DNA, 20 bp, synthetic construct
  misc_feature 1..20: note = Synthetic
  source 1..20: mol_type = other DNA
gtgggaactttgagaaccgc

SEQ ID NO: 985 - DNA, 20 bp, synthetic construct
  misc_feature 1..20: note = Synthetic
  source 1..20: mol_type = other DNA
ttctcaaagttcccacatcc

SEQ ID NO: 986 - DNA, 20 bp, synthetic construct
  misc_feature 1..20: note = Synthetic
  source 1..20: mol_type = other DNA
aaagttcccacatcctggtg

SEQ ID NO: 987 - DNA, 20 bp, synthetic construct
  misc_feature 1..20: note = Synthetic
  source 1..20: mol_type = other DNA
aagttcccacatcctggtgc

SEQ ID NO: 988 - DNA, 20 bp, synthetic construct
  misc_feature 1..20: note = Synthetic
  source 1..20: mol_type = other DNA
cccacatcctggtgcggggc

SEQ ID NO: 989 - DNA, 20 bp, synthetic construct
  misc_feature 1..20: note = Synthetic
  source 1..20: mol_type = other DNA
cctgccccgcaccaggatgt

SEQ ID NO: 990 - DNA, 20 bp, synthetic construct
  misc_feature 1..20: note = Synthetic
  source 1..20: mol_type = other DNA
tcctgccccgcaccaggatg

SEQ ID NO: 991 - DNA, 20 bp, synthetic construct
  misc_feature 1..20: note = Synthetic
  source 1..20: mol_type = other DNA
ctgcgctcctgccccgcacc

SEQ ID NO: 992 - DNA, 20 bp, synthetic construct
  misc_feature 1..20: note = Synthetic
  source 1..20: mol_type = other DNA
cggggcaggagcgcagcctt

SEQ ID NO: 993 - DNA, 20 bp, synthetic construct
  misc_feature 1..20: note = Synthetic
  source 1..20: mol_type = other DNA
atctgtgcaggggataccga

SEQ ID NO: 994 - DNA, 20 bp, synthetic construct
  misc_feature 1..20: note = Synthetic
  source 1..20: mol_type = other DNA
cgacaaacttatctgtgcag

SEQ ID NO: 995 - DNA, 20 bp, synthetic construct
  misc_feature 1..20: note = Synthetic
  source 1..20: mol_type = other DNA
acgacaaacttatctgtgca

SEQ ID NO: 996 - DNA, 20 bp, synthetic construct
  misc_feature 1..20: note = Synthetic
  source 1..20: mol_type = other DNA
gacgacaaacttatctgtgc

SEQ ID NO: 997 - DNA, 20 bp, synthetic construct
  misc_feature 1..20: note = Synthetic
  source 1..20: mol_type = other DNA
tttgtcgtcttttcacagat

SEQ ID NO: 998 - DNA, 20 bp, synthetic construct
  misc_feature 1..20: note = Synthetic
  source 1..20: mol_type = other DNA
tcacagattggtgagtagcc

SEQ ID NO: 999 - DNA, 20 bp, synthetic construct
  misc_feature 1..20: note = Synthetic
  source 1..20: mol_type = other DNA
cacagattggtgagtagccc

SEQ ID NO: 1000 - DNA, 20 bp, synthetic construct
  misc_feature 1..20: note = Synthetic
  source 1..20: mol_type = other DNA
cactttgcagtcatgttggg

SEQ ID NO: 1001 - DNA, 20 bp, synthetic construct
  misc_feature 1..20: note = Synthetic
  source 1..20: mol_type = other DNA
ttacactttgcagtcatgtt

SEQ ID NO: 1002 - DNA, 20 bp, synthetic construct
  misc_feature 1..20: note = Synthetic
  source 1..20: mol_type = other DNA
attacactttgcagtcatgt

SEQ ID NO: 1003 - DNA, 20 bp, synthetic construct
  misc_feature 1..20: note = Synthetic
  source 1..20: mol_type = other DNA
agacacattcctttatgcac

SEQ ID NO: 1004 - DNA, 20 bp, synthetic construct
  misc_feature 1..20: note = Synthetic
  source 1..20: mol_type = other DNA
tctcattttccagtgcataa

SEQ ID NO: 1005 - DNA, 20 bp, synthetic construct
  misc_feature 1..20: note = Synthetic
  source 1..20: mol_type = other DNA
ctatgagcttcagatacaaa

SEQ ID NO: 1006 - DNA, 20 bp, synthetic construct
  misc_feature 1..20: note = Synthetic
  source 1..20: mol_type = other DNA
gcagcctgtaatcacagaac

SEQ ID NO: 1007 - DNA, 20 bp, synthetic construct
  misc_feature 1..20: note = Synthetic
  source 1..20: mol_type = other DNA
ctcacctgttctgtgattac

SEQ ID NO: 1008 - DNA, 20 bp, synthetic construct
  misc_feature 1..20: note = Synthetic
  source 1..20: mol_type = other DNA
tttattttctttcaaaccac

SEQ ID NO: 1009 - DNA, 20 bp, synthetic construct
  misc_feature 1..20: note = Synthetic
  source 1..20: mol_type = other DNA
gaggttctgtctctgacctg

SEQ ID NO: 1010 - DNA, 20 bp, synthetic construct
  misc_feature 1..20: note = Synthetic
  source 1..20: mol_type = other DNA
tccttccagctactcaatcc

SEQ ID NO: 1011 - DNA, 20 bp, synthetic construct
  misc_feature 1..20: note = Synthetic
  source 1..20: mol_type = other DNA
aggattgagtagctggaagg

SEQ ID NO: 1012 - DNA, 20 bp, synthetic construct
  misc_feature 1..20: note = Synthetic
  source 1..20: mol_type = other DNA
tccaggattgagtagctgga

SEQ ID NO: 1013 - DNA, 20 bp, synthetic construct
  misc_feature 1..20: note = Synthetic
  source 1..20: mol_type = other DNA
acgttccaggattgagtagc

SEQ ID NO: 1014 - DNA, 20 bp, synthetic construct
  misc_feature 1..20: note = Synthetic
  source 1..20: mol_type = other DNA
atttgtactgtgtacgttcc

SEQ ID NO: 1015 - DNA, 20 bp, synthetic construct
  misc_feature 1..20: note = Synthetic
  source 1..20: mol_type = other DNA
acacagtacaaataagagcc

SEQ ID NO: 1016 - DNA, 20 bp, synthetic construct
  misc_feature 1..20: note = Synthetic
  source 1..20: mol_type = other DNA
cacagtacaaataagagccc

SEQ ID NO: 1017 - DNA, 20 bp, synthetic construct
  misc_feature 1..20: note = Synthetic
  source 1..20: mol_type = other DNA
gaattcatacactctttccc

SEQ ID NO: 1018 - DNA, 20 bp, synthetic construct
  misc_feature 1..20: note = Synthetic
  source 1..20: mol_type = other DNA
agaattcatacactctttcc

SEQ ID NO: 1019 - DNA, 20 bp, synthetic construct
  misc_feature 1..20: note = Synthetic
  source 1..20: mol_type = other DNA
tgtatgaattcttgagcgcc

SEQ ID NO: 1020 - DNA, 20 bp, synthetic construct
  misc_feature 1..20: note = Synthetic
  source 1..20: mol_type = other DNA
tggagcaccccccagcgctt

SEQ ID NO: 1021 - DNA, 20 bp, synthetic construct
  misc_feature 1..20: note = Synthetic
  source 1..20: mol_type = other DNA
gaagcgctggggggtgctcc

SEQ ID NO: 1022 - DNA, 20 bp, synthetic construct
  misc_feature 1..20: note = Synthetic
  source 1..20: mol_type = other DNA
ccccccagcgcttcggtgag

SEQ ID NO: 1023 - DNA, 20 bp, synthetic construct
  misc_feature 1..20: note = Synthetic
  source 1..20: mol_type = other DNA
cccccagcgcttcggtgagt

SEQ ID NO: 1024 - DNA, 20 bp, synthetic construct
  misc_feature 1..20: note = Synthetic
  source 1..20: mol_type = other DNA
ccactcaccgaagcgctggg

SEQ ID NO: 1025 - DNA, 20 bp, synthetic construct
  misc_feature 1..20: note = Synthetic
  source 1..20: mol_type = other DNA
cccactcaccgaagcgctgg

SEQ ID NO: 1026 - DNA, 20 bp, synthetic construct
  misc_feature 1..20: note = Synthetic
  source 1..20: mol_type = other DNA
gcccactcaccgaagcgctg

SEQ ID NO: 1027 - DNA, 20 bp, synthetic construct
  misc_feature 1..20: note = Synthetic
  source 1..20: mol_type = other DNA
agcccactcaccgaagcgct

SEQ ID NO: 1028 - DNA, 20 bp, synthetic construct
  misc_feature 1..20: note = Synthetic
  source 1..20: mol_type = other DNA
cagcccactcaccgaagcgc

SEQ ID NO: 1029 - DNA, 20 bp, synthetic construct
  misc_feature 1..20: note = Synthetic
  source 1..20: mol_type = other DNA
gcttcggtgagtgggctgtg

SEQ ID NO: 1030 - DNA, 20 bp, synthetic construct
  misc_feature 1..20: note = Synthetic
  source 1..20: mol_type = other DNA
cttcggtgagtgggctgtgc

SEQ ID NO: 1031 - DNA, 20 bp, synthetic construct
  misc_feature 1..20: note = Synthetic
  source 1..20: mol_type = other DNA
ttcggtgagtgggctgtgcg

SEQ ID NO: 1032 - DNA, 20 bp, synthetic construct
  misc_feature 1..20: note = Synthetic
  source 1..20: mol_type = other DNA
tctaggggtaaagggtgaga

SEQ ID NO: 1033 - DNA, 20 bp, synthetic construct
  misc_feature 1..20: note = Synthetic
  source 1..20: mol_type = other DNA
ctctaggggtaaagggtgag

SEQ ID NO: 1034 - DNA, 20 bp, synthetic construct
  misc_feature 1..20: note = Synthetic
  source 1..20: mol_type = other DNA
tttacccctagagtgcgacc

SEQ ID NO: 1035 - DNA, 20 bp, synthetic construct
  misc_feature 1..20: note = Synthetic
  source 1..20: mol_type = other DNA
ggtcgcactctaggggtaaa

SEQ ID NO: 1036 - DNA, 20 bp, synthetic construct
  misc_feature 1..20: note = Synthetic
  source 1..20: mol_type = other DNA
tggtcgcactctaggggtaa

SEQ ID NO: 1037 - DNA, 20 bp, synthetic construct
  misc_feature 1..20: note = Synthetic
  source 1..20: mol_type = other DNA
acccctagagtgcgaccagg

SEQ ID NO: 1038 - DNA, 20 bp, synthetic construct
  misc_feature 1..20: note = Synthetic
  source 1..20: mol_type = other DNA
cctagagtgcgaccaggagg

SEQ ID NO: 1039 - DNA, 20 bp, synthetic construct
  misc_feature 1..20: note = Synthetic
  source 1..20: mol_type = other DNA
ctagagtgcgaccaggagga

SEQ ID NO: 1040 - DNA, 20 bp, synthetic construct
  misc_feature 1..20: note = Synthetic
  source 1..20: mol_type = other DNA
tcctcctggtcgcactctag

SEQ ID NO: 1041 - DNA, 20 bp, synthetic construct
  misc_feature 1..20: note = Synthetic
  source 1..20: mol_type = other DNA
ctcctcctggtcgcactcta

SEQ ID NO: 1042 - DNA, 20 bp, synthetic construct
  misc_feature 1..20: note = Synthetic
  source 1..20: mol_type = other DNA
cctcctcctggtcgcactct

SEQ ID NO: 1043 - DNA, 20 bp, synthetic construct
  misc_feature 1..20: note = Synthetic
  source 1..20: mol_type = other DNA
gtgtgtttgcgccctcctcc

SEQ ID NO: 1044 - DNA, 20 bp, synthetic construct
  misc_feature 1..20: note = Synthetic
  source 1..20: mol_type = other DNA
agggcgcaaacacacgtgcc

SEQ ID NO: 1045 - DNA, 20 bp, synthetic construct
  misc_feature 1..20: note = Synthetic
  source 1..20: mol_type = other DNA
gcgcaaacacacgtgcctgg

SEQ ID NO: 1046 - DNA, 20 bp, synthetic construct
  misc_feature 1..20: note = Synthetic
  source 1..20: mol_type = other DNA
gatcagcagcgacgtccgcc

SEQ ID NO: 1047 - DNA, 20 bp, synthetic construct
  misc_feature 1..20: note = Synthetic
  source 1..20: mol_type = other DNA
gacgtcgctgctgatcgcgc

SEQ ID NO: 1048 - DNA, 20 bp, synthetic construct
  misc_feature 1..20: note = Synthetic
  source 1..20: mol_type = other DNA
acgtcgctgctgatcgcgct

SEQ ID NO: 1049 - DNA, 20 bp, synthetic construct
  misc_feature 1..20: note = Synthetic
  source 1..20: mol_type = other DNA
cgtcgctgctgatcgcgctg

SEQ ID NO: 1050 - DNA, 20 bp, synthetic construct
  misc_feature 1..20: note = Synthetic
  source 1..20: mol_type = other DNA
gatcgcgctggggacgctgc

SEQ ID NO: 1051 - DNA, 20 bp, synthetic construct
  misc_feature 1..20: note = Synthetic
  source 1..20: mol_type = other DNA
gctggggacgctgctggccc

SEQ ID NO: 1052 - DNA, 20 bp, synthetic construct
  misc_feature 1..20: note = Synthetic
  source 1..20: mol_type = other DNA
gatcacgaagacacagacca

SEQ ID NO: 1053 - DNA, 20 bp, synthetic construct
  misc_feature 1..20: note = Synthetic
  source 1..20: mol_type = other DNA
agatcacgaagacacagacc

SEQ ID NO: 1054 - DNA, 20 bp, synthetic construct
  misc_feature 1..20: note = Synthetic
  source 1..20: mol_type = other DNA
gtgtcttcgtgatctgcaga

SEQ ID NO: 1055 - DNA, 20 bp, synthetic construct
  misc_feature 1..20: note = Synthetic
  source 1..20: mol_type = other DNA
ctgcagaaggtgagccctcg

SEQ ID NO: 1056 - DNA, 20 bp, synthetic construct
  misc_feature 1..20: note = Synthetic
  source 1..20: mol_type = other DNA
tgcagaaggtgagccctcga

SEQ ID NO: 1057 - DNA, 20 bp, synthetic construct
  misc_feature 1..20: note = Synthetic
  source 1..20: mol_type = other DNA
ggccatttctctttcctccg

SEQ ID NO: 1058 - DNA, 20 bp, synthetic construct
  misc_feature 1..20: note = Synthetic
  source 1..20: mol_type = other DNA
tacctcggaggaaagagaaa

SEQ ID NO: 1059 - DNA, 20 bp, synthetic construct
  misc_feature 1..20: note = Synthetic
  source 1..20: mol_type = other DNA
tctctttcctccgaggtatc

SEQ ID NO: 1060 - DNA, 20 bp, synthetic construct
  misc_feature 1..20: note = Synthetic
  source 1..20: mol_type = other DNA
tgcatcaccagatacctcgg

SEQ ID NO: 1061 - DNA, 20 bp, synthetic construct
  misc_feature 1..20: note = Synthetic
  source 1..20: mol_type = other DNA
ctctgcatcaccagatacct

SEQ ID NO: 1062 - DNA, 20 bp, synthetic construct
  misc_feature 1..20: note = Synthetic
  source 1..20: mol_type = other DNA
tctttcatgtgagggatgcg

SEQ ID NO: 1063 - DNA, 20 bp, synthetic construct
  misc_feature 1..20: note = Synthetic
  source 1..20: mol_type = other DNA
gtctttcatgtgagggatgc

SEQ ID NO: 1064 - DNA, 20 bp, synthetic construct
  misc_feature 1..20: note = Synthetic
  source 1..20: mol_type = other DNA
ggtctttcatgtgagggatg

SEQ ID NO: 1065 - DNA, 20 bp, synthetic construct
  misc_feature 1..20: note = Synthetic
  source 1..20: mol_type = other DNA
cctcacatgaaagaccccat

SEQ ID NO: 1066 - DNA, 20 bp, synthetic construct
  misc_feature 1..20: note = Synthetic
  source 1..20: mol_type = other DNA
cgatggggtctttcatgtga

SEQ ID NO: 1067 - DNA, 20 bp, synthetic construct
  misc_feature 1..20: note = Synthetic
  source 1..20: mol_type = other DNA
ccgatggggtctttcatgtg

SEQ ID NO: 1068 - DNA, 20 bp, synthetic construct
  misc_feature 1..20: note = Synthetic
  source 1..20: mol_type = other DNA
tttggaagctgtcaccgatg

SEQ ID NO: 1069 - DNA, 20 bp, synthetic construct
  misc_feature 1..20: note = Synthetic
  source 1..20: mol_type = other DNA
ttttggaagctgtcaccgat

SEQ ID NO: 1070 - DNA, 20 bp, synthetic construct
  misc_feature 1..20: note = Synthetic
  source 1..20: mol_type = other DNA
gttttggaagctgtcaccga

SEQ ID NO: 1071 - DNA, 20 bp, synthetic construct
  misc_feature 1..20: note = Synthetic
  source 1..20: mol_type = other DNA
cagcttccaaaacgacaagc

SEQ ID NO: 1072 - DNA, 20 bp, synthetic construct
  misc_feature 1..20: note = Synthetic
  source 1..20: mol_type = other DNA
aacataccagcttgtcgttt

SEQ ID NO: 1073 - DNA, 20 bp, synthetic construct
  misc_feature 1..20: note = Synthetic
  source 1..20: mol_type = other DNA
ctgcctcctctcgtctctgc

SEQ ID NO: 1074 - DNA, 20 bp, synthetic construct
  misc_feature 1..20: note = Synthetic
  source 1..20: mol_type = other DNA
cctcctctcgtctctgcagg

SEQ ID NO: 1075 - DNA, 20 bp, synthetic construct
  misc_feature 1..20: note = Synthetic
  source 1..20: mol_type = other DNA
ccacctgcagagacgagagg

SEQ ID NO: 1076 - DNA, 20 bp, synthetic construct
  misc_feature 1..20: note = Synthetic
  source 1..20: mol_type = other DNA
tctcgtctctgcaggtggtc

SEQ ID NO: 1077 - DNA, 20 bp, synthetic construct
  misc_feature 1..20: note = Synthetic
  source 1..20: mol_type = other DNA
ctcgtctctgcaggtggtct

SEQ ID NO: 1078 - DNA, 20 bp, synthetic construct
  misc_feature 1..20: note = Synthetic
  source 1..20: mol_type = other DNA
agaccacctgcagagacgag

SEQ ID NO: 1079 - DNA, 20 bp, synthetic construct
  misc_feature 1..20: note = Synthetic
  source 1..20: mol_type = other DNA
gtctctgcaggtggtctggg

SEQ ID NO: 1080 - DNA, 20 bp, synthetic construct
  misc_feature 1..20: note = Synthetic
  source 1..20: mol_type = other DNA
tctgcaggtggtctgggagg

SEQ ID NO: 1081 - DNA, 20 bp, synthetic construct
  misc_feature 1..20: note = Synthetic
  source 1..20: mol_type = other DNA
ctgcaggtggtctgggaggc

SEQ ID NO: 1082 - DNA, 20 bp, synthetic construct
  misc_feature 1..20: note = Synthetic
  source 1..20: mol_type = other DNA
gtctgggaggcgggcaaagc

SEQ ID NO: 1083 - DNA, 20 bp, synthetic construct
  misc_feature 1..20: note = Synthetic
  source 1..20: mol_type = other DNA
ggaggcgggcaaagccggcc

SEQ ID NO: 1084 - DNA, 20 bp, synthetic construct
  misc_feature 1..20: note = Synthetic
  source 1..20: mol_type = other DNA
ggcgggcaaagccggcctgg

SEQ ID NO: 1085 - DNA, 20 bp, synthetic construct
  misc_feature 1..20: note = Synthetic
  source 1..20: mol_type = other DNA
agccggcctggaggagtgtc

SEQ ID NO: 1086 - DNA, 20 bp, synthetic construct
  misc_feature 1..20: note = Synthetic
  source 1..20: mol_type = other DNA
caccagacactcctccaggc

SEQ ID NO: 1087 - DNA, 20 bp, synthetic construct
  misc_feature 1..20: note = Synthetic
  source 1..20: mol_type = other DNA
cagtcaccagacactcctcc

SEQ ID NO: 1088 - DNA, 20 bp, synthetic construct
  misc_feature 1..20: note = Synthetic
  source 1..20: mol_type = other DNA
gtgtctggtgactgaagtac

SEQ ID NO: 1089 - DNA, 20 bp, synthetic construct
  misc_feature 1..20: note = Synthetic
  source 1..20: mol_type = other DNA
tcgtgcagaaaacttgagac

SEQ ID NO: 1090 - DNA, 20 bp, synthetic construct
  misc_feature 1..20: note = Synthetic
  source 1..20: mol_type = other DNA
cgtgcagaaaacttgagact

SEQ ID NO: 1091 - DNA, 20 bp, synthetic construct
  misc_feature 1..20: note = Synthetic
  source 1..20: mol_type = other DNA
gtgcagaaaacttgagactg

SEQ ID NO: 1092 - DNA, 20 bp, synthetic construct
  misc_feature 1..20: note = Synthetic
  source 1..20: mol_type = other DNA
aaaacttgagactggggttc

SEQ ID NO: 1093 - DNA, 20 bp, synthetic construct
  misc_feature 1..20: note = Synthetic
  source 1..20: mol_type = other DNA
aaacttgagactggggttca

SEQ ID NO: 1094 - DNA, 20 bp, synthetic construct
  misc_feature 1..20: note = Synthetic
  source 1..20: mol_type = other DNA
agactggggttcagggcttg

SEQ ID NO: 1095 - DNA, 20 bp, synthetic construct
  misc_feature 1..20: note = Synthetic
  source 1..20: mol_type = other DNA
gacctgctggattcatgacg

SEQ ID NO: 1096 - DNA, 20 bp, synthetic construct
  misc_feature 1..20: note = Synthetic
  source 1..20: mol_type = other DNA
gtcgtactggacgtccgcgg

SEQ ID NO: 1097 - DNA, 20 bp, synthetic construct
  misc_feature 1..20: note = Synthetic
  source 1..20: mol_type = other DNA
ggtcgtactggacgtccgcg

SEQ ID NO: 1098-1099 - skipped (2 entries)

SEQ ID NO: 1100 - DNA, 20 bp, synthetic construct
  misc_feature 1..20: note = Synthetic
  source 1..20: mol_type = other DNA
actcacctatccacatgcag

SEQ ID NO: 1101 - DNA, 21 bp, synthetic construct
  misc_feature 1..21: note = Synthetic
  source 1..21: mol_type = other DNA
tcaagatttctttacatattc

SEQ ID NO: 1102 - DNA, 20 bp, synthetic construct
  misc_feature 1..20: note = Synthetic
  source 1..20: mol_type = other DNA
gacactcacctatccacatg

SEQ ID NO: 1103 - DNA, 20 bp, synthetic construct
  misc_feature 1..20: note = Synthetic
  source 1..20: mol_type = other DNA
ctcacctatccacatgcaga

SEQ ID NO: 1104 - DNA, 20 bp, synthetic construct
  misc_feature 1..20: note = Synthetic
  source 1..20: mol_type = other DNA
cttctgcatgtggataggtg

SEQ ID NO: 1105 - DNA, 20 bp, synthetic construct
  misc_feature 1..20: note = Synthetic
  source 1..20: mol_type = other DNA
tctgcatgtggataggtgag

SEQ ID NO: 1106 - DNA, 20 bp, synthetic construct
  misc_feature 1..20: note = Synthetic
  source 1..20: mol_type = other DNA
acactcacctatccacatgc

SEQ ID NO: 1107 - DNA, 20 bp, synthetic construct
  misc_feature 1..20: note = Synthetic
  source 1..20: mol_type = other DNA
aaaggtaagattttgagtta

SEQ ID NO: 1108 - DNA, 21 bp, synthetic construct
  misc_feature 1..21: note = Synthetic
  source 1..21: mol_type = other DNA
cacagcacctccagctccctc

SEQ ID NO: 1109 - DNA, 21 bp, synthetic construct
  misc_feature 1..21: note = Synthetic
  source 1..21: mol_type = other DNA
aatgtggagttcacgtaactt

SEQ ID NO: 1110 - DNA, 21 bp, synthetic construct
  misc_feature 1..21: note = Synthetic
  source 1..21: mol_type = other DNA
atgtggagttcacgtaacttt

SEQ ID NO: 1111 - DNA, 20 bp, synthetic construct
  misc_feature 1..20: note = Synthetic
  source 1..20: mol_type = other DNA
aaggtaatcttgtattctct

SEQ ID NO: 1112 - DNA, 21 bp, synthetic construct
  misc_feature 1..21: note = Synthetic
  source 1..21: mol_type = other DNA
ctgtaggaatttataaaatcc

SEQ ID NO: 1113 - DNA, 20 bp, synthetic construct
  misc_feature 1..20: note = Synthetic
  source 1..20: mol_type = other DNA
cttacctttttccccaaatt

SEQ ID NO: 1114 - DNA, 20 bp, synthetic construct
  misc_feature 1..20: note = Synthetic
  source 1..20: mol_type = other DNA
aaggtaagattttgagttat

SEQ ID NO: 1115 - DNA, 20 bp, synthetic construct
  misc_feature 1..20: note = Synthetic
  source 1..20: mol_type = other DNA
agtacatacacatgcttgcc

SEQ ID NO: 1116 - DNA, 21 bp, synthetic construct
  misc_feature 1..21: note = Synthetic
  source 1..21: mol_type = other DNA
tctttcagggttataagaaac

SEQ ID NO: 1117 - DNA, 20 bp, synthetic construct
  misc_feature 1..20: note = Synthetic
  source 1..20: mol_type = other DNA
gtggagttcacgtaactttg

SEQ ID NO: 1118 - DNA, 20 bp, synthetic construct
  misc_feature 1..20: note = Synthetic
  source 1..20: mol_type = other DNA
ttaccttgttctttgctata

SEQ ID NO: 1119 - DNA, 20 bp, synthetic construct
  misc_feature 1..20: note = Synthetic
  source 1..20: mol_type = other DNA
ggtaatcttgtattctcttg

SEQ ID NO: 1120 - DNA, 20 bp, synthetic construct
  misc_feature 1..20: note = Synthetic
  source 1..20: mol_type = other DNA
ttcttccagagcacaaatgt

SEQ ID NO: 1121 - DNA, 20 bp, synthetic construct
  misc_feature 1..20: note = Synthetic
  source 1..20: mol_type = other DNA
gtacatacacatgcttgcca

SEQ ID NO: 1122 - DNA, 20 bp, synthetic construct
  misc_feature 1..20: note = Synthetic
  source 1..20: mol_type = other DNA
atgtggagttcacgtaactt

SEQ ID NO: 1123 - DNA, 20 bp, synthetic construct
  misc_feature 1..20: note = Synthetic
  source 1..20: mol_type = other DNA
tggagttcacgtaactttga

SEQ ID NO: 1124 - DNA, 20 bp, synthetic construct
  misc_feature 1..20: note = Synthetic
  source 1..20: mol_type = other DNA
tgtaggaatttataaaatcc

SEQ ID NO: 1125 - DNA, 20 bp, synthetic construct
  misc_feature 1..20: note = Synthetic
  source 1..20: mol_type = other DNA
tggtaagtgtctattcttgt

SEQ ID NO: 1126 - DNA, 20 bp, synthetic construct
  misc_feature 1..20: note = Synthetic
  source 1..20: mol_type = other DNA
aggtaatcttgtattctctt

SEQ ID NO: 1127 - DNA, 20 bp, synthetic construct
  misc_feature 1..20: note = Synthetic
  source 1..20: mol_type = other DNA
tcttccagagcacaaatgta

SEQ ID NO: 1128 - DNA, 20 bp, synthetic construct
  misc_feature 1..20: note = Synthetic
  source 1..20: mol_type = other DNA
ctttaccaatgcatttttgt

SEQ ID NO: 1129 - DNA, 20 bp, synthetic construct
  misc_feature 1..20: note = Synthetic
  source 1..20: mol_type = other DNA
gggctttaccaatgcatttt

SEQ ID NO: 1130 - DNA, 20 bp, synthetic construct
  misc_feature 1..20: note = Synthetic
  source 1..20: mol_type = other DNA
gtaggaatttataaaatccc

SEQ ID NO: 1131 - DNA, 20 bp, synthetic construct
  misc_feature 1..20: note = Synthetic
  source 1..20: mol_type = other DNA
cacttaccaggcagaggagt

SEQ ID NO: 1132 - DNA, 20 bp, synthetic construct
  misc_feature 1..20: note = Synthetic
  source 1..20: mol_type = other DNA
gcctggtaagtgtctattct

SEQ ID NO: 1133 - DNA, 20 bp, synthetic construct
  misc_feature 1..20: note = Synthetic
  source 1..20: mol_type = other DNA
ggtaagtgtctattcttgtt

SEQ ID NO: 1134 - DNA, 20 bp, synthetic construct
  misc_feature 1..20: note = Synthetic
  source 1..20: mol_type = other DNA
tctttcagggttataagaaa

SEQ ID NO: 1135 - DNA, 20 bp, synthetic construct
  misc_feature 1..20: note = Synthetic
  source 1..20: mol_type = other DNA
ctgcatgtggataggtgagt

SEQ ID NO: 1136 - DNA, 21 bp, synthetic construct
  misc_feature 1..21: note = Synthetic
  source 1..21: mol_type = other DNA
gaatatgtaaagaaatcttga

SEQ ID NO: 1137 - DNA, 20 bp, synthetic construct
  misc_feature 1..20: note = Synthetic
  source 1..20: mol_type = other DNA
catgtggataggtgagtgtc

SEQ ID NO: 1138 - DNA, 20 bp, synthetic construct
  misc_feature 1..20: note = Synthetic
  source 1..20: mol_type = other DNA
tctgcatgtggataggtgag

SEQ ID NO: 1139 - DNA, 20 bp, synthetic construct
  misc_feature 1..20: note = Synthetic
  source 1..20: mol_type = other DNA
cacctatccacatgcagaag

SEQ ID NO: 1140 - DNA, 20 bp, synthetic construct
  misc_feature 1..20: note = Synthetic
  source 1..20: mol_type = other DNA
ctcacctatccacatgcaga

SEQ ID NO: 1141 - DNA, 20 bp, synthetic construct
  misc_feature 1..20: note = Synthetic
  source 1..20: mol_type = other DNA
gcatgtggataggtgagtgt

SEQ ID NO: 1142 - DNA, 20 bp, synthetic construct
  misc_feature 1..20: note = Synthetic
  source 1..20: mol_type = other DNA
taactcaaaatcttaccttt

SEQ ID NO: 1143 - DNA, 21 bp, synthetic construct
  misc_feature 1..21: note = Synthetic
  source 1..21: mol_type = other DNA
gagggagctggaggtgctgtg

SEQ ID NO: 1144 - DNA, 21 bp, synthetic construct
  misc_feature 1..21: note = Synthetic
  source 1..21: mol_type = other DNA
aagttacgtgaactccacatt

SEQ ID NO: 1145 - DNA, 21 bp, synthetic construct
  misc_feature 1..21: note = Synthetic
  source 1..21: mol_type = other DNA
aaagttacgtgaactccacat

SEQ ID NO: 1146 - DNA, 20 bp, synthetic construct
  misc_feature 1..20: note = Synthetic
  source 1..20: mol_type = other DNA
agagaatacaagattacctt

SEQ ID NO: 1147 - DNA, 21 bp, synthetic construct
  misc_feature 1..21: note = Synthetic
  source 1..21: mol_type = other DNA
ggattttataaattcctacag

SEQ ID NO: 1148 - DNA, 20 bp, synthetic construct
  misc_feature 1..20: note = Synthetic
  source 1..20: mol_type = other DNA
aatttggggaaaaaggtaag

SEQ ID NO: 1149 - DNA, 20 bp, synthetic construct
  misc_feature 1..20: note = Synthetic
  source 1..20: mol_type = other DNA
ataactcaaaatcttacctt

SEQ ID NO: 1150 - DNA, 20 bp, synthetic construct
  misc_feature 1..20: note = Synthetic
  source 1..20: mol_type = other DNA
ggcaagcatgtgtatgtact

SEQ ID NO: 1151 - DNA, 21 bp, synthetic construct
  misc_feature 1..21: note = Synthetic
  source 1..21: mol_type = other DNA
gtttcttataaccctgaaaga

SEQ ID NO: 1152 - DNA, 20 bp, synthetic construct
  misc_feature 1..20: note = Synthetic
  source 1..20: mol_type = other DNA
caaagttacgtgaactccac

SEQ ID NO: 1153 - DNA, 20 bp, synthetic construct
  misc_feature 1..20: note = Synthetic
  source 1..20: mol_type = other DNA
tatagcaaagaacaaggtaa

SEQ ID NO: 1154 - DNA, 20 bp, synthetic construct
  misc_feature 1..20: note = Synthetic
  source 1..20: mol_type = other DNA
caagagaatacaagattacc

SEQ ID NO: 1155 - DNA, 20 bp, synthetic construct
  misc_feature 1..20: note = Synthetic
  source 1..20: mol_type = other DNA
acatttgtgctctggaagaa

SEQ ID NO: 1156 - DNA, 20 bp, synthetic construct
  misc_feature 1..20: note = Synthetic
  source 1..20: mol_type = other DNA
tggcaagcatgtgtatgtac

SEQ ID NO: 1157 - DNA, 20 bp, synthetic construct
  misc_feature 1..20: note = Synthetic
  source 1..20: mol_type = other DNA
aagttacgtgaactccacat

SEQ ID NO: 1158 - DNA, 20 bp, synthetic construct
  misc_feature 1..20: note = Synthetic
  source 1..20: mol_type = other DNA
tcaaagttacgtgaactcca

SEQ ID NO: 1159 - DNA, 20 bp, synthetic construct
  misc_feature 1..20: note = Synthetic
  source 1..20: mol_type = other DNA
ggattttataaattcctaca

SEQ ID NO: 1160 - DNA, 20 bp, synthetic construct
  misc_feature 1..20: note = Synthetic
  source 1..20: mol_type = other DNA
acaagaatagacacttacca

SEQ ID NO: 1161 - DNA, 20 bp, synthetic construct
  misc_feature 1..20: note = Synthetic
  source 1..20: mol_type = other DNA
aagagaatacaagattacct

SEQ ID NO: 1162 - DNA, 20 bp, synthetic construct
  misc_feature 1..20: note = Synthetic
  source 1..20: mol_type = other DNA
tacatttgtgctctggaaga

SEQ ID NO: 1163 - DNA, 20 bp, synthetic construct
  misc_feature 1..20: note = Synthetic
  source 1..20: mol_type = other DNA
acaaaaatgcattggtaaag

SEQ ID NO: 1164 - DNA, 20 bp, synthetic construct
  misc_feature 1..20: note = Synthetic
  source 1..20: mol_type = other DNA
aaaatgcattggtaaagccc

SEQ ID NO: 1165 - DNA, 20 bp, synthetic construct
  misc_feature 1..20: note = Synthetic
  source 1..20: mol_type = other DNA
gggattttataaattcctac

SEQ ID NO: 1166 - DNA, 20 bp, synthetic construct
  misc_feature 1..20: note = Synthetic
  source 1..20: mol_type = other DNA
actcctctgcctggtaagtg

SEQ ID NO: 1167 - DNA, 20 bp, synthetic construct
  misc_feature 1..20: note = Synthetic
  source 1..20: mol_type = other DNA
agaatagacacttaccaggc

SEQ ID NO: 1168 - DNA, 20 bp, synthetic construct
  misc_feature 1..20: note = Synthetic
  source 1..20: mol_type = other DNA
aacaagaatagacacttacc

SEQ ID NO: 1169 - DNA, 20 bp, synthetic construct
  misc_feature 1..20: note = Synthetic
  source 1..20: mol_type = other DNA
tttcttataaccctgaaaga

SEQ ID NO: 1170 - RNA, 20 nt, synthetic construct
  misc_feature 1..20: note = Synthetic
  source 1..20: mol_type = other RNA
actcacctatccacatgcag

SEQ ID NO: 1171 - RNA, 21 nt, synthetic construct
  misc_feature 1..21: note = Synthetic
  source 1..21: mol_type = other RNA
tcaagatttctttacatattc

SEQ ID NO: 1172 - RNA, 20 nt, synthetic construct
  misc_feature 1..20: note = Synthetic
  source 1..20: mol_type = other RNA
gacactcacctatccacatg

SEQ ID NO: 1173 - RNA, 20 nt, synthetic construct
  misc_feature 1..20: note = Synthetic
  source 1..20: mol_type = other RNA
ctcacctatccacatgcaga

SEQ ID NO: 1174 - RNA, 20 nt, synthetic construct
  misc_feature 1..20: note = Synthetic
  source 1..20: mol_type = other RNA
cttctgcatgtggataggtg

SEQ ID NO: 1175 - RNA, 20 nt, synthetic construct
  misc_feature 1..20: note = Synthetic
  source 1..20: mol_type = other RNA
tctgcatgtggataggtgag

SEQ ID NO: 1176 - RNA, 20 nt, synthetic construct
  misc_feature 1..20: note = Synthetic
  source 1..20: mol_type = other RNA
acactcacctatccacatgc

SEQ ID NO: 1177 - RNA, 20 nt, synthetic construct
  misc_feature 1..20: note = Synthetic
  source 1..20: mol_type = other RNA
aaaggtaagattttgagtta

SEQ ID NO: 1178 - RNA, 21 nt, synthetic construct
  misc_feature 1..21: note = Synthetic
  source 1..21: mol_type = other RNA
cacagcacctccagctccctc

SEQ ID NO: 1179 - RNA, 21 nt, synthetic construct
  misc_feature 1..21: note = Synthetic
  source 1..21: mol_type = other RNA
aatgtggagttcacgtaactt

SEQ ID NO: 1180 - RNA, 21 nt, synthetic construct
  misc_feature 1..21: note = Synthetic
  source 1..21: mol_type = other RNA
atgtggagttcacgtaacttt

SEQ ID NO: 1181 - RNA, 20 nt, synthetic construct
  misc_feature 1..20: note = Synthetic
  source 1..20: mol_type = other RNA
aaggtaatcttgtattctct

SEQ ID NO: 1182 - RNA, 21 nt, synthetic construct
  misc_feature 1..21: note = Synthetic
  source 1..21: mol_type = other RNA
ctgtaggaatttataaaatcc

SEQ ID NO: 1183 - RNA, 20 nt, synthetic construct
  misc_feature 1..20: note = Synthetic
  source 1..20: mol_type = other RNA
cttacctttttccccaaatt

SEQ ID NO: 1184 - RNA, 20 nt, synthetic construct
  misc_feature 1..20: note = Synthetic
  source 1..20: mol_type = other RNA
aaggtaagattttgagttat

SEQ ID NO: 1185 - RNA, 20 nt, synthetic construct
  misc_feature 1..20: note = Synthetic
  source 1..20: mol_type = other RNA
agtacatacacatgcttgcc

SEQ ID NO: 1186 - RNA, 21 nt, synthetic construct
  misc_feature 1..21: note = Synthetic
  source 1..21: mol_type = other RNA
tctttcagggttataagaaac

SEQ ID NO: 1187 - RNA, 20 nt, synthetic construct
  misc_feature 1..20: note = Synthetic
  source 1..20: mol_type = other RNA
gtggagttcacgtaactttg

SEQ ID NO: 1188 - RNA, 20 nt, synthetic construct
  misc_feature 1..20: note = Synthetic
  source 1..20: mol_type = other RNA
ttaccttgttctttgctata

SEQ ID NO: 1189 - RNA, 20 nt, synthetic construct
  misc_feature 1..20: note = Synthetic
  source 1..20: mol_type = other RNA
ggtaatcttgtattctcttg

SEQ ID NO: 1190 - RNA, 20 nt, synthetic construct
  misc_feature 1..20: note = Synthetic
  source 1..20: mol_type = other RNA
ttcttccagagcacaaatgt

SEQ ID NO: 1191 - RNA, 20 nt, synthetic construct
  misc_feature 1..20: note = Synthetic
  source 1..20: mol_type = other RNA
gtacatacacatgcttgcca

SEQ ID NO: 1192 - RNA, 20 nt, synthetic construct
  misc_feature 1..20: note = Synthetic
  source 1..20: mol_type = other RNA
atgtggagttcacgtaactt

SEQ ID NO: 1193 - RNA, 20 nt, synthetic construct
  misc_feature 1..20: note = Synthetic
  source 1..20: mol_type = other RNA
tggagttcacgtaactttga

SEQ ID NO: 1194 - RNA, 20 nt, synthetic construct
  misc_feature 1..20: note = Synthetic
  source 1..20: mol_type = other RNA
tgtaggaatttataaaatcc

SEQ ID NO: 1195 - RNA, 20 nt, synthetic construct
  misc_feature 1..20: note = Synthetic
  source 1..20: mol_type = other RNA
tggtaagtgtctattcttgt

SEQ ID NO: 1196 - RNA, 20 nt, synthetic construct
  misc_feature 1..20: note = Synthetic
  source 1..20: mol_type = other RNA
aggtaatcttgtattctctt

SEQ ID NO: 1197 - RNA, 20 nt, synthetic construct
  misc_feature 1..20: note = Synthetic
  source 1..20: mol_type = other RNA
tcttccagagcacaaatgta

SEQ ID NO: 1198 - RNA, 20 nt, synthetic construct
  misc_feature 1..20: note = Synthetic
  source 1..20: mol_type = other RNA
ctttaccaatgcatttttgt

SEQ ID NO: 1199 - RNA, 20 nt, synthetic construct
  misc_feature 1..20: note = Synthetic
  source 1..20: mol_type = other RNA
gggctttaccaatgcatttt

SEQ ID NO: 1200 - RNA, 20 nt, synthetic construct
  misc_feature 1..20: note = Synthetic
  source 1..20: mol_type = other RNA
gtaggaatttataaaatccc

SEQ ID NO: 1201 - RNA, 20 nt, synthetic construct
  misc_feature 1..20: note = Synthetic
  source 1..20: mol_type = other RNA
cacttaccaggcagaggagt

SEQ ID NO: 1202 - RNA, 20 nt, synthetic construct
  misc_feature 1..20: note = Synthetic
  source 1..20: mol_type = other RNA
gcctggtaagtgtctattct

SEQ ID NO: 1203 - RNA, 20 nt, synthetic construct
  misc_feature 1..20: note = Synthetic
  source 1..20: mol_type = other RNA
ggtaagtgtctattcttgtt

SEQ ID NO: 1204 - RNA, 20 nt, synthetic construct
  misc_feature 1..20: note = Synthetic
  source 1..20: mol_type = other RNA
tctttcagggttataagaaa

SEQ ID NO: 1205-1999 - skipped (795 entries)

SEQ ID NO: 2000 - DNA, 20 bp, synthetic construct
  misc_feature 1..20: note = Synthetic
  source 1..20: mol_type = other DNA
atgctcacatgaagaagatg

SEQ ID NO: 2001 - DNA, 20 bp, synthetic construct
  misc_feature 1..20: note = Synthetic
  source 1..20: mol_type = other DNA
cactcacctgcccacagcag

SEQ ID NO: 2002 - DNA, 20 bp, synthetic construct
  misc_feature 1..20: note = Synthetic
  source 1..20: mol_type = other DNA
catcttcttcatgtgagcat

SEQ ID NO: 2003 - DNA, 20 bp, synthetic construct
  misc_feature 1..20: note = Synthetic
  source 1..20: mol_type = other DNA
ctgctgtgggcaggtgagtg

SEQ ID NO: 2004 - DNA, 54 bp, synthetic construct
  misc_feature 1..54: note = Synthetic
  source 1..54: mol_type = other DNA
tgtctctgcctcatcttcttcatgtgagcattttctctgggtcaggcatgggcc

SEQ ID NO: 2005 - DNA, 20 bp, synthetic construct
  misc_feature 1..20: note = Synthetic
  source 1..20: mol_type = other DNA
cctctacctgtgcagagctt

SEQ ID NO: 2006 - DNA, 20 bp, synthetic construct
  misc_feature 1..20: note = Synthetic
  source 1..20: mol_type = other DNA
ctctacctgtgcagagcttc

SEQ ID NO: 2007 - DNA, 20 bp, synthetic construct
  misc_feature 1..20: note = Synthetic
  source 1..20: mol_type = other DNA
ccttacctggatggtgttat

SEQ ID NO: 2008 - DNA, 20 bp, synthetic construct
  misc_feature 1..20: note = Synthetic
  source 1..20: mol_type = other DNA
tgtaccgtcacaagtctcca

SEQ ID NO: 2009 - DNA, 20 bp, synthetic construct
  misc_feature 1..20: note = Synthetic
  source 1..20: mol_type = other DNA
agcactcacacggtgggaga

SEQ ID NO: 2010 - DNA, 20 bp, synthetic construct
  misc_feature 1..20: note = Synthetic
  source 1..20: mol_type = other DNA
atcttaccttgtgttccgga

SEQ ID NO: 2011 - DNA, 20 bp, synthetic construct
  misc_feature 1..20: note = Synthetic
  source 1..20: mol_type = other DNA
gtggtacctgctggctgagg

SEQ ID NO: 2012 - DNA, 20 bp, synthetic construct
  misc_feature 1..20: note = Synthetic
  source 1..20: mol_type = other DNA
aagctctgcacaggtagagg

SEQ ID NO: 2013 - DNA, 20 bp, synthetic construct
  misc_feature 1..20: note = Synthetic
  source 1..20: mol_type = other DNA
gaagctctgcacaggtagag

SEQ ID NO: 2014 - DNA, 20 bp, synthetic construct
  misc_feature 1..20: note = Synthetic
  source 1..20: mol_type = other DNA
ataacaccatccaggtaagg

SEQ ID NO: 2015 - DNA, 20 bp, synthetic construct
  misc_feature 1..20: note = Synthetic
  source 1..20: mol_type = other DNA
tggagacttgtgacggtaca

SEQ ID NO: 2016 - DNA, 20 bp, synthetic construct
  misc_feature 1..20: note = Synthetic
  source 1..20: mol_type = other DNA
tctcccaccgtgtgagtgct

SEQ ID NO: 2017 - DNA, 20 bp, synthetic construct
  misc_feature 1..20: note = Synthetic
  source 1..20: mol_type = other DNA
tccggaacacaaggtaagat

SEQ ID NO: 2018 - DNA, 20 bp, synthetic construct
  misc_feature 1..20: note = Synthetic
  source 1..20: mol_type = other DNA
cctcagccagcaggtaccac

SEQ ID NO: 2019 - protein, 823 aa, Homo sapiens
MGGRVFLVFLAFCVWLTLPGAETQDSRGCARWCPQDSSCVNATACRCNPGFSSFSEIITTPMETCDDINECATLSKVSCGKFSDCWNTEGSYDCVCSPGYEPVSGAKTFKNESENTCQDVDECQQNPRLCKSYGTCVNTLGSYTCQCLPGFKLKPEDPKLCTDVNECTSGQNPCHSSTHCLNNVGSYQCRCRPGWQPIPGSPNGPNNTVCEDVDECSSGQHQCDSSTVCFNTVGSYSCRCRPGWKPRHGIPNNQKDTVCEDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQEEDPVLTVITYMGLSVSLLCLLLAALTFLLCKAIQNTSTSLHLQLSLCLFLAHLLFLVAIDQTGHKVLCSIIAGTLHYLYLATLTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

SEQ ID NO: 2020 - DNA, 6791 bp, Homo sapiens
  source 1..6791: mol_type = genomic DNA
agaaagaccagaaagatccggggggtctgctctgtgcggtgaagcttctcttcttggcacctgcctggcatcggaagagggccccttctccctccctgggcttttatgtggacactgtaatgcctcagttttctttctttctttttgtttttgacacagggtctcactctatcacccaagctggagtgcagtggcaccatcttagctcaccgcagcctcaaactccccagctcgggtgattcttcctgcctcagcctcctgagtagtagcagctgggaccacagacgtctgccaccaagcccagctgcaccgccagttccggggagggccctgggccagcggctgtccgccccccctcctttataaagtcctggcctcgggacagcccgcacagctgcccagcctgcggagacgggacagccctgtcccactcactctttcccctgctgctcctgccggcagctcagctggaaccatgggaggccgcgtctttctcgtctttctcgcattctgtgtctggctgactctgccgggagctgaaacccaggactccaggggctgtgcccggtggtgccctcaggactcctcgtgtgtcaatgccaccgcctgtcgctgcaatccagggttcagctctttttctgagatcatcaccacccccatggagacttgtgacgacatcaacgagtgtgcaacactgtcgaaagtgtcatgcggaaaattctcggactgctggaacacagaggggagctacgactgcgtgtgcagcccaggatatgagcctgtttctggggcaaaaacattcaagaatgagagcgagaacacgtgtcaagatgtggacgaatgtcagcagaacccaaggctctgtaaaagctacggcacctgcgtcaacaccctcggcagctacacgtgccagtgcctgcctggcttcaagctcaaacctgaggacccgaagctctgcacagatgtgaatgaatgcacctccggacaaaacccatgccacagctccacccactgcctcaacaacgtgggcagctatcagtgccgctgccgcccgggctggcaaccgattccggggtcccccaatggcccaaacaataccgtctgtgaagatgtggacgagtgcagctccgggcagcatcagtgtgacagctccaccgtctgcttcaacaccgtgggttcatacagctgccgctgccgcccaggctggaagcccagacacggaatcccgaataaccaaaaggacactgtctgtgaagatatgactttctccacctggaccccgccccctggagtccacagccagacgctttcccgattcttcgacaaagtccaggacctgggcagagactacaagccaggcttggccaataacaccatccagagcatcttacaggcgctggatgagctgctggaggcccctggggacctggagaccctgccccgcttacagcagcactgtgtggccagtcacctgctggatggcctagaggatgtcctcagaggcctgagcaagaacctttccaatgggctgttgaacttcagttatcctgcaggcacagaattgtccctggaggtgcagaagcaagtagacaggagtgtcaccttgagacagaatcaggcagtgatgcagctcgactggaatcaggcacagaaatctggtgacccaggcccttctgtggtgggccttgtctccattccagggatgggcaagttgctggctgaggcccctctggtcctggaacctgagaagcagatgcttctgcatgagacacaccagggcttgctgcaggacggctcccccatcctgctctcagatgtgatctctgcctttctgagcaacaacgacacccaaaacctcagctccccagttaccttcaccttctcccaccgttcagtgatcccgagacagaaggtgctctgtgtcttctgggagcatggccagaatggatgtggtcactgggccaccacaggctgcagcacaataggcaccagagacaccagcaccatctgccgttgcacccacctgagcagctttgccgtcctcatggcccactacgatgtgcaggaggaggatcccgtgctgactgtcatcacctacatggggctgagcgtctctctgctgtgcctcctcctggcggccctcacttttctcctgtgtaaagccatccagaacaccagcacctcactgcatctgcagctctcgctctgcctcttcctggcccacctcctcttcctcgtggcaattgatcaaaccggacacaaggtgctgtgctccatcatcgccggtaccttgcactatctctacctggccaccttgacctggatgctgctggaggccctgtacctcttcctcactgcacggaacctgacggtggtcaactactcaagcatcaacagattcatgaagaagctcatgttccctgtgggctacggagtcccagctgtgacagtggccatttctgcagcctccaggcctcacctttatggaacaccttcccgctgctggctccaaccagaaaagggatttatatggggcttccttggacctgtctgcgccatcttctctgtgaatttagttctctttctggtgactctctggattttgaaaaacagactctcctccctcaatagtgaagtgtccaccctccggaacacaaggatgctggcatttaaagcgacagctcagctgttcatcctgggctgcacgtggtgtctgggcatcttgcaggtgggtccggctgcccgggtcatggcctacctcttcaccatcatcaacagcctgcagggtgtcttcatcttcctggtgtactgcctcctcagccagcaggtccgggagcaatatgggaaatggtccaaagggatcaggaaattgaaaactgagtctgagatgcacacactctccagcagtgctaaggctgacacctccaaacccagcacggttaactagaaaaatcttctgaataagatcttccctctttgcccgtggaaaatctgaacaatctttgagccatctagaggggaaagaaaagactttgttctgtgtgtttcaagaaattcaccatgtcagcaatatgaaggatgttatggaaggcgtgctaggcattcaattcctgcagaaaccggaaatcttccatgccctgcaatgtgctcatcaaactctcagcatatggacggccagctgtggcccatatcttggtcactctgaagcacaatatttatgaagctatagaacgttaagacctctttcacagcctctccttcctacaaagactcctccaaatcttaaaatgaagcaggaaaacgagcctaagaggactttcataccgacaacatctgaaaggactagaatgttcacaccacgatctggatttcttaattttttgtttttgtttttgttgttctctagttctacgggtttgattatttagtcatgtgaaaaatattgattactcacacatagatcaagagagacacggctcctgccttcatggagcttttaggggaaaatgaagtggctcttgcagctagagttgactcagaagccgaaattcctagaaatcaggtttctactgctaggcaattgaagtataaactattttataaacactgtcttctttcgtcttcacaccaacatgcagaaaagtttctaatctcagatcggggatgtgcaacaaattccatttcaaaggaatgacctgcaaaactcctaaatattccaagcaaatgcccttaaccctgtctgttatctgctttccttgaacagaaattctacatgaccataaaacctcgaagatgggtatggcacagttcatgccctgtaatcctagcactttgggagggtgaggcaggaggatggctcaagcccaggagtttgagaccagtgtgggcaacagagtgagaaccatctctacccaaaaaaaaaattaaaaattagccaagcatggtgatgatataggagttaaggagaaatcatttaggcaaatagcaagggtaggaagtcctcagtaaggttttccatttaatgaaaagcagcccccaaaatcattttcttttctaacaaagaacagcctgtaaaatcgagctgcagacatagacaagcaagctggaagcttccacgggtgaatgccggcagctgtgccaataggaaaaagctacctagactaggcatgtccaaaatggcggctccaagttcccttctctttgccagccatgtgtacagtaaaaagcaggcaacatagtgtcagccaaagctcatttgcataataagattagggtggggtggccagctcacataggggtaggccctaggtaaatcagacaccgccttctcaagcctgtctataaaatctggtacactatgacgagggtcagatttcccattcagacgcccctctcccatgcaagagaaagagctgttctcctttctctttcttttgcctattaaacctctgctcctggccaggcacagtggctcacgcctataatcccagcactttgggaggctgaggtggtcagatcacctaaggtcaggagttcaagaccagcctggtcaacatggtgaaatcttgtctctagtaaaaatacaaaaatatatgaaatctcacatagatgataatattaagttccaaaagcaactcaacctggtagattctaattttttttgaggcagggtcttgctttgtcacccatgctggagtacaatggcacaaacactgctcactgcagcctcgacctcccaaggcctaagcaatcctcctgcctcagtccccctccaggtatttgaaactacaggtgtgtaccaccacaccgggctaatttttgtattttttgtagagacgtgggtctcactatgctgcccaggctcaggtcttaatctcctgagctcaggcaatccgcaggcctcagcctccctaagtgcggggattacaggcttgagccactgcacctagcctctatttgttttacaaaagagaaattgagatcctgaatgttaagtgacttgcctgaggccatcccactaacaggagccagggttaggattcaaaccccatccaactggtcccagagctggagcttcttgcactgccctacactacctaccatctccatcctctgggcacctttttataagaaccaaaacattacagagcattgctttgtcaactcagctgggaacatttcccagtgcaactcacatttttcactgctctgtgcctgtccgtataagctcaatgagtattgatttaggggctttggagaactttgaatgctaccccccaagtaaccattgttggcaacctggtacctctacttttagccatttctccttctctataaatagtgcagaagtaacccacttggtaacaggcatccttgccaagcctccaccactaggtcagtgtaagaattaaagaaagaggaaagaaacacaaaaagtggcttgatggttaagacaggtttattttagagaaaacacacctgagaggggctgctggctgaattaggttagagtcttttctacagactaagagtgtttaaggatttagggtgggagagtttcttagaggcttggactgcttctgtgttttttttgttgtgcttatatgggagggagagtggtgtgtttgcttttatacatttttctgcagctgtaggcataccccccaagtctgcttttagcttccctattttagtgcacctggagggaaaggaatgtgcttattaaggcccactgttttactggggcccattgtatgagggtgaagtttggcagttacccaagagacttttcctccaccttcctctgtgcccgagctgttttatctgcattttactgtctgctttttttggctgcttatagtttttaaaaaagtaatttccttaaatccagaaggctaaaaatgaagctgaaacttaaagtggcggtgtttgtccaaaataacggggctcctgctctgccagtcagtaccctcaagtcactcctgatcctcaacctccatgcctaaggctggttcaagagaccacataatatctgccttttattacatacatgatgggtgcatgggattctgcgtgccctttgcttgatatagactgctaaggtgagatggggaatatcagagtcagctgctgcttgaggaagcagaacacacagctggaggcttggaacatgtgggtccctatgagtgtagagcccatatccccatagagtctacctagagcaggggtcgccaaatgttttcttaaagagcctgatagtgtatatgttaggctttgtgagccaggtatttacagcaactcaattctaccactgtggtatgaaaacagctatagacaatcataaatgaatgatcatggctatgttttaataaaactttacagacactgaacttgaacttccattgtgatatgaaaacagctatagacaatcataaatgaatgatcatggctatgttttaataaaactttatggacactgagcttgaacttcatatatcagtcatgtgacacaaaatatcattcttctttttatttgttttcacccatttgaaaaatgtaaaaactattcttagctgtacagaaacagatggtgggtcagatttgactcactgcccatagtttccagaccatgatgttcaggttcatggaagcacttcactctacactaatttattcatccattcatcacatatgtgatgagcacatacagacacacctcagagatattgcaggctccactccagatcaccacaataatgtgaatattgcaataaagcaagtcatacacattt

SEQ ID NO: 2021 - DNA, 32767 bp, Homo sapiens
  source 1..32767: mol_type = genomic DNA
agaaagaccagaaagatccggggggtctgctctgtgcggtgaagcttctcttcttggcacctgcctggcatcggaagagggccccttctccctccctgggcttttatgtggacactgtaatgcctcagttttctttctttctttttgtttttgacacagggtctcactctatcacccaagctggagtgcagtggcaccatcttagctcaccgcagcctcaaactccccagctcgggtgattcttcctgcctcagcctcctgagtagtagcagctgggaccacagacgtctgccaccaagcccaggtaattttttttctttttgaagaaacagagctttgccatgttgcccaggctggtctcaaactcctgagctcaagcgatcctcctgcctcggcctcccagagtgctgggatcacaggtgtggagaaataggaacgcttttacactgttggtgggaatgtaaattagttcaaccattgtggaagatagtgtggtgatttctcaaggatccagaactagaaataccatttgactcagcgatcccattgctgggtatatacccaaaggattataaatcatgctactataaagacatatgcacatgtatgtttattgcggtactattcacaatagcaaagacctggaatcaacccaaatgtccatcaatgatagactggattaagaaagtgtggcacatatacaccagggaatactatgcagctataaaaaaggatgagttcatgtcctttgcagggacacagatgaagctgaaaaccatcattctcagcaaactaacacagaaacagaaaaccaaacaccacatgttctcactcataggtgggaactgagcaatgagaacacctggacacagggcgggaaacatcacacactggggcctgtcggggggtgggggcctgggggagggattgcattaggagaaatacctaatgtaaatgacgagttgataggtgcagcaagccaacatggcacatgtatgcctatgtatcaaacctgctcgttgtgcacatgtaccctagaacttaaagtctaaaaaatttaattaattaattaattaattaatttaaaaagaaagaaagaaaccgagcttcgccatgttgcccaggctggtctcaaactcctgagctcaagcgatcctcctgcctcgccctcccagagtgctgggatcacaggcgtgagcctctgtacctggcctcagtttcctcatctggaaaacagcaggggaagatactgccagctgggacgcaggctctgccaactctggcatgccaaagctctgggcacagggtgagccctaccaaagtgggagcccagagctatttctgacaaggcccctgcgtgcctgcccagggaagagccaagtggccccaggcgagaccgccccgcccggctgccacaccttcccctgcacagcagccagcctggaacacacagaggccagagggttggtcagagccacatggtggaaactccagcagagggatttttaaagcaggttgcacttcattcttgctgagcgagtacacgtgtgtgtgtgtgtgtgtgtgtgtgtgtgtgtgtgtgtgcagcgcccctggttctgtgtttttattgtgctttcctgctggcttccagctgcaccgccagttccggggagggccctgggccagcggctgtccgccccccctcctttataaagtcctggcctcgggacagcccgcacagctgcccagcctgcggagacgggacagccctgtcccactcactctttcccctgctgctcctgccggcagctcagctggaaccatgggaggccgcgtctttctcgtctttctcggtaagtactttggggccccgctgggggtggtagcgaggaagctccagcgggaccccttggatgcgtctgagaaacgggaggcgccgctggtgtctgtggggccggcgatggagaaagccacatggtcaagcaggcactcacgggcacacggcgagaggggctggtggtgcttgggcacccccacaccccggcaggaactccagggtgctctcggagcctgctaccctgcccggagggatcctcaagctttctactggagccagtttggcatctgctccataagaggggaaactgaggcacgggggaaggaaaggggcaaaaagtgagtgctaggaccccagagccactgctcttaatatagcgcatagaaagacatgggcggggggcggggaaaatagaggccaggacttctccactgcagggtacccccagcttctcccttctttctctctctctctctgtctttctccttctgtctctctttcagtctctctatctccctaatctccgttttctctctacctctgcctctttgtctctctctctggtgaacaacaaggagatccctaggtccccatctctaaagcccctggtgacctgaggagggtcccccaggacatgccttggaaaggggaacttcctgagtgctgggaaccgcagcgccacatgcaccccaacaggcggccacagcgttttcctgtgggaggggcgcccggggtgaaatatcaccactgggtttcaggatgggccgccagggctggcggcggttgtggtgtgtgtgcacctgtgtccctgaggaagcatgcggttccagggcatcttagagcctgggggatccagcgccatcatttttttttttttttttctgaggcggagccttgctctgtcgcccaggctggagtgcagtggcacgatctcagcttgctgcaacctccgcctcctgggttcaagcgattctcctgcctcaatctcccaagtagttgggtaacaggagtctgccaccacgcccagctaatgttttgtatttttagtagagacggggtttcaccatattgggcaggctggtctcgaactcccgacctcagatgatctgcccaccttggcctcccaaagtgctgggattacagtcccgagccaccgtgcctgaccatgccttcatttttatctaaagaaatgaagatggcagtaaatgctcaggcacaccagacagcccccaagtcagaagagcggcagctggagctgagacccccaccaggctcatggccctttcctactcctcagttccttaaaccccacccccaagccaagctagggaggctgaggcaggaggatcgcttgaggccaggagttcaagatcagcctgggcaacagagcaagactctgtctctaaaataactttttaaattatttttaatttttttggccaggcgtgatggctcatgcctgtaatcccagcactttgagaggccaaggcgggcagatcacctgaggttaggagttcaagaccagcctggccaacatggtgaaaccccatctctacttaagaaatacaaaaattagctgggcatggtgggtcatgcctgtagtcccagccactcaagaggctgaggcaggagaaccgcttgaacccaggaggtggagattgcagtgagccaagattgcaccactgcattccagcctgggcaacaaagggagactccatctcaaaaaaaaataaataataaaaataaataaatcattttaaaatttttgagctgggagccgtggctcacacctgtaatcctagcactttgggaggccaaggccggcagatcacctgaggtcaggagttcgagaccagcctggccaatatggtgaaaccccatctcactaaaaatacaaaaaattagccaggcaccatggtgggtgcctgtagtcccagctatttgggaggttgaggcaggagaattgcttgaacccaggaggtagagattgcaattagccgagatcgcaccattgcacttcagcctgggcgacagagcaagaccctgtctcaaaaagaaagaaagaaaatttaaaatatggcggtactcacctgtggtcccagctactcgggagggtgaggcagggggatcgcttgagcctaggaattggaggctgcagtgagctataatcgcatcactgcaccccagcctgggtgacccagagagaccctgtgtcaaacaaaacaaaacaaaacccacccccatgaggacagggtgacagactctctggctttgagcatccgagtgggacgtgacatctgctcactctgtttgaacgatgggcaggatctgagcctgagaggggtcagcatcctcctcccacaaagtgcatcacccttacgcctcctttcccaccctgggatcccctctgaccccctttcctttctctgttgcagcattctgtgtctggctgactctgccgggagctgaaacccaggactccaggggtgagtctgctgggaagcagaaagcacagtccacagccagagcctggggagggtcctggacccccgcccagcccccttcagcccagggaaagagagggctcgtgcacgggaaactcagcgctctgccccatctcccccagtgccccctttttgtgtattcccttacccctcaccttctgaccgtgctccctgctcttgcaggctgtgcccggtggtgccctcaggactcctcgtgtgtcaatgccaccgcctgtcgctgcaatccagggttcagctctttttctgagatcatcaccacccccatggagacttgtgacggtacagaggcttgagggcagcgcaggggacatctgcgattatgaggcatagcccagtgccagtgggggacagaggttgttgtgaggggccacagccttaccttccagactatcatcatggccagagaaaagagaaagagggcaggtgtggtggctcccagcactttgggaggcagaggcgggtggatcatttgaggccaggaattcaagaccagcctgggcaacatggtgagaccctgtctctacaaaaaatacacaaaatagccgcgcatggtggcacacatctggagtcccagctactcagaagactgaggcgggaggattgtttgagcccaggagtttgaggttgcagtgagctatgattgcaccattgcactctagcctgggcaacagagtgagatcctgtctcaggaaaaagaaagaaacaaagaaagaggccaggtgtggtggctcatgcctgtaatcccagcactttgggagggattgtctgggcaacaggagcaagaccccatctcaaaaaaaaaaaaaaaagacagaaagagagaaggagagaaagaaaagagaagaaaagaaaaagagagagagggagggagggaaagaaaaaggaaaggagggagggagggagggaggaaagaaggaaggaaggaagggaggaaaaaggaaaaaatatgaagggggagaagtaagaagtgaataggcatggcttcctggagagagagaagctgcgtgctcaggaatctggagtctgtgcctcagtttaccgttaagactgggaagggggtacattctggccataggtttttttttctcccttatttttcttttttctttttttttttgttttttttttttttttttgagatggagtctcactctgtcgctcaggctgaggagtgcagtggcgcgatctcggctcaccgcaacctccacctcccaggttcaagcgattcttctgcctcagcctcccgagtagctgggattacaggcatgcaccaccatgcccggctagtttttgtatttttagtagagatggggttttgccctgttggcaggctggtctcgaactcctgacctcaagtgatctgccggcctcggcctcccaaagtgctgggattacaggtatgagccactgcacccggctgccatggggtttaaatttctacaaaaagactggggatggggatatggcgcttcctgtccatcccccacctcagcactgtccactgtgtccaaatctaggatgccagccagagaactgagatcaagtgtggtcttcaggaatggcctgctccagctgcagcatagcctgtgcagtgcaacttgagatcctttccaaaagatactaagcagcaggccccatgttggggagtcaggacgggggtctctgccctgacggaactcacatctttgggagatgacctcctgtcctgttgtgttccagacatcaacgagtgtgcaacactgtcgaaagtgtcatgcggaaaattctcggactgctggaacacagaggggagctacgactgcgtgtgcagcccaggatatgagcctgtttctggggcaaaaacattcaagaatgagagcgagaacacgtgtcaaggtaagaaccaccccacatcctccatcaccactgtccatgaggtttggggtcaccagagctgtttctgcagcatccagggagcaggtacccaagtataggttcagttcctggagtctgagatgagacaggtgtgcatgtacctgtttcgccagcccagagaggcagcgtggtgtgaaggccaagggaaaactctgggaccagctacctgggcccaaatcctggctttgccacctgttacctaaatgatattaccaacatcactactgctcttagagccttggtttacccatctataaaatgaggatgataataatggcctactcctatgccttctaggaggaccaaggaaagaataggtgcttctaacagagcccagcacacagcaggtgctacaggcatcttcattctttaaacgttattatgcactgtaatcccagcactttgggaggccgaggcggatggatcacggggtcaggagatcgagactatcctggccaacatggtgaaaccctgtctctactaaaagtacgaaaattagccgggtgcagtggtgggtgcctgtaatcccagctactcaggaggctgaggcaggagaatcacttgaaccagggagtcggaggtttcagtgagcggagatcgtgccactgcactccagcctggtgacagtgcaagactccatctaaaataaataaataaataaataaataaataaataaataagcaatggatgccttttaggagttgttcagacaagctgggctcctgtggggccacactcctggctggcactcagaagaaatctatttcgacacagcctgcctcatcccttgccatggtcttattccacactgcagctccagcccatgagaagccaggcccccatgcatcactctgctttgccttctttccagtgggaactagttgggtgcgggagggagcttcagacatctgcaaggccagatgggaaccagtagcaggtggacagagaagggagattaacacagagtacacagagtgggaagggaaggtgagcaaaggcaggaaatgaagaagctgaggctgagccaagggtcccaggggacattaagggggttcagcagacctgccctgctgtaaagagaaaagtggcagtgctcctgttcccagccctgtccctcctgttctccctgcagtcagtagagcctccctggtgggaggaaagtgaggacagagcagaggtgtacgttctgagagcacgcagctgcatagagcccagcaggggggctggttcacaggggtcccctgtgaacagatgctacattgcatgcacatatcttgaccaaaggacagagccttcctgtgaattagaaaaaggactctccgggccaggcacagtggctcacgcctgtaatcccagcactttgggaggctgaggcgggtggatcacctgaggtcaggagttcaagaccagcctgacaaacatagtgaaaccccatctctactaaaaaatacaaaagttagccaggcatagtggtgagcacctgtaatcccagctactcaggaggctgaggcaggagaatcacctgaacccagaggcagaggttgcagtgagccaggatctcaatattgcactccagcctgggtgacagagcaagactttgtctcaaaaaaaaaaaaaaaaaaaaaaaaaaaagaaaagaaagaaacaaaaagaaaaagagaaacagaaaagcctctcctctccaggtagacacagccccatttggggaatggagaggttttcagcctctgctcaccgccttggctgggcatccatatgtagtcagcaacctacacaactgtacgtggtggtcatgggtccttgtcagtgagagcctctgttttagtctatgttgtgttgctataaaggaatacatgaggctggatcatgtataaagaaaagaagctgatttggctcatggttctgcaggccatgtaagacacatggctccagcatctgcttctggtgagggccttagggagcttccactcttggcagacgggaaaggggaagctggcatcccatggcaagaggaaggaagcaagagagaagggaggagataccaggctttttccaacaagcagttttcatgggaactaacaaggcgagaactcactcatgacccaaggacagcaccgagatgttcatgagggatctgcactcaggacccaaacacctcccacgaggctccatctccaacactggggatcacatttcaacgtgagatttggaggggacaaatatccaaactgtatcaccgcatgcacaggtggtgtttgtaattattgaggaggtgtgttgccatgttagaaacaggccagcactggccgggcgcagtggctcatgcctgtaatcctagcactttgggaggccgaggtgggcggatcacaaggtcaggagatcgagaccatcctggctaacatggtgaaaccccgtctctactacaaatacaaaaaaattagctgggcgtggtggtgggtgcctgtagtcccagctaatcagagactgaggcaggagaatggcatgaacctgggaggcggagcttgcagtaagccgagatggcaccactgcactccagcctggacgacagagcgagactccgtctccaaaaaaaagaaaagaaaaggagagaagagaagagagaaaagaaaagaggccaacactgagagagggggcttagttggtggataggcagagacacagatgagcaggaatgaagtgggagagggctctcatggaccccatccgcacagaacagcctgctagttcggagcaggacctgcctcttgaagcctcagctcagggaagggctccgtggggccctgtctgcatccccctcacccctcagcattcttgttttttttgtctacctggtgtcacaggcctcataggcaggagacctggggccactccctcagctccttgccccacttactccaggcatcacagggccgtcccaaacacctggtcacctgtcgctatccaaacctcaaggttcagaaccctccacagagggtcacttccaaaagctcagaacatccccctgcccatctggcatcttatttgtttgttttgttttgttttattagtaaagatggggggggggtgtctcaccatgttgtccaggctggtctcgaactcctggcctcaagtgatccgcccgtctcagcctcccaaagtgctgggattgcaggcgtgagccaccatgtccgacctggtatcttatttgtacaaaacaaaaagggggctggcactcaggatattcgatgagctcactgcgtggtgcattccagcgcagtatccattctctctgctatcatcagcgactgccaggatgagacagtctccacacagctctgctcacctggaactctctctgatcccccagctcatcatggacccaggcaccctgtccagaaatgaaggaggagggagatttgaaggcagggccactgtccccagtcgaacctgccactggccaacaaccccacctggagaagatgcctcgtggagccctgacctctttttctgccactcagtctctgtccctttcactgagcgagactgccctgacccttcctgatagttttggtccacctgagtgcactggaggatgttgtggctaacggtgggcacagtgacatggactccccatacttggagagttgagtgaatggaagaggcactagtgtggacccaatggcaagagaggttaacatcagggccattgctccaagcctgatcctctcccaggaggtcaacccagatcccatgggagtggaaccattcagaaataggtctgtgtccctggtggaatgccaagagatgagatgacagatacacctccatctttgcccatcagcatctgtgcccctctccagccactaaaccttcacccagggccagtctttctcagctatggattctcactacatcctgccatttccaagacaccttcctgaggatcaaaaaaaggcaccttcacagccctggggcaacccagctgtatgtcggtccccactgtccaccctggcctgggacccactgtccaggcagccctggcctatgcagcaccagactcatgctcccctggggactggctcaggggccagtcttggtccatgtgcctggagctgggctctcagagctagtgtggatgggagagcaccatcctgctagggccaccgggattctgataactcagccagtgagcagctgtcctggggaaatcaaggccaccagaagcagatgctggagccatgcatcttacacagcctgcagaaccatgagccaaataaacttcttttcttgcaacacaaaacggactaaaccagagggttctcactgagaaggacccatgaccaccatgtacagttgcataggctgctgactgcatatggatgcccagccaagagggtgagcagaagctgaaaacctgcctactctccattcctcaaatatgactgtgactacttgaagaggagatgcctttttccaattcacaggaaggctctgtcctttggcccaggactgccactgggaaccaagacaggtcctacctgggcaagcagcatgccccctgcacttggtcccaagaaaatcccaccctgacctttgtcctgaatcctcaggctgggacatccctgccccagctgggagctgcccagggaggcaggggagatggggaggactcagatgcttccagccatatctgaacaacagaaggcctgggccaccatgctcaccatgggacacctctgtgtggattagaaaagggaggagatggccaggcatggtggctcatgcctgcaatcccagcactttgggaggccgaggtgggctgatcacgaggtcaggagatcaagaccatcctggctaacacggtgaaaccccgtctaatactaaaaatacaaaaaattagccgggcatggtggcgggcgcctgtagtcccagctattcgggaggctgaggcaggagaatggcgtgaacccgggaggcggaggttgcagtgagttgagatcaggcaattgcactccagcctgggtgacagagcaagactccgtctcaaaaaaaaaaaaaagaaagaaagaaaagaaaagggagcagacagtggtgccttccagatgcttccgtgccaggaaacatggcggaccctcagccccgaaagctgctcacagctacgggcattcttcaccctctccttcctcttgcagatgtggacgaatgtcagcagaacccaaggctctgtaaaagctacggcacctgcgtcaacaccctcggcagctacacgtgccagtgcctgcctggcttcaagctcaaacctgaggacccgaagctctgcacaggtagaggccccaggaagacgctgtgaggctggacgggagctggggatggagctgagtcaggtcctccaaagcagccgaggaggagggagaagatccgcaggttcccacaaggtcaaggacctgctaagcccctgcctaaggattcacctcccaggaaggactcgccacacggcagggaggcggcagggccttcagggcttggagtgcccttgtgggcccccagacctcaccccttcctcatctgtcacgtgcgagtggaagaaggtgtcacttccagttctaagaaggggaacctccaccatagtgagggagggagcaggagagagattttagtcagaaagtccagctcccatgactcagtttcccttttggtttcacctcctataaactggggaccatggtccctgctgtgcccatctggtaggaagaccatcataagatgctttgaaggtgaagttgaaggtcaccaagtggtggcacgtgcaataatggtcttcagtctgaggcaatgaaaagataggggagtgggtgcagtgagtggagggcaggcccaggctggtcagggacagggtttgaccctctggctttgtcctcagatgtgaatgaatgcacctccggacaaaacccatgccacagctccacccactgcctcaacaacgtgggcagctatcagtgccgctgccgcccgggctggcaaccgattccggggtcccccaatggcccaaacaataccgtctgtgaaggtcgagagctcagatcccacgttcccagagacccacaaacatctgatcacatgttcaacggcgcccacacaaaccaagcagaatgagcgctggaggcgcccgactgtgtcaggcgttcattcttctgaggctagatgagaaaagagcaagggtcctgcggaaggagctggggtactgaggggggaggctcagggggaccccaggcaacagctgatgactcactgggaggaaggcgtttcaccatattcataacctgctcatctgcacggggcccacctgctgtgcccaggcctctccacgcttccataacccagcgtccacctctccaaggggggcactaatgccgggaggaacgagctgggggcacagacaggagacaggaccctctccaggctgggacaggacctgacccccttcttcctgtcctcagatgtggacgagtgcagctccgggcagcatcagtgtgacagctccaccgtctgcttcaacaccgtgggttcatacagctgccgctgccgcccaggctggaagcccagacacggaatcccgaataaccaaaaggacactgtctgtgaaggtatgacctggccctagaagctccccacccccagcacacacactgacacgctcccgcctaatgagccgcttgtcttgttccctacagatatgactttctccacctggaccccgccccctggagtccacagccaggtgagtggcccccacagggacgaggcggcgggaactccatccacacagcactgcatccgtctccttgttctaaacttcccacccgccgtccaggctctctgacccccacatctcctctctctgcagacgctttcccgattcttcgacaaagtccaggacctgggcagagactacaagccaggcttggccaataacaccatccaggtaaggacaggacccagggcaagggggcgaggcaggaaggtggctgcatcccacagaggcctggggcagtttgggtctgggaggggacaggacccaatgcagtgggtgctggtctgactcccagcatctttcagggctggtggaagctaatggacacccaagtgcacttaatatctttcctcttgctttccctggactttgggtttaggtcagcattgatattgcacagaagtgttggcctctgaggggccataaccagagtcaaggatcacctagggaatcccatccaatgtcattttttttttagacggagtctcactctgtcgccagtccagagtttagtggcccgatctcagctcactgcagtctctgcctcccgggttcaagcgattctcctccctcagccccctgagtagctaggattacaggcacacgccaccacgcccggctaatttttgtatttttagtagagacggggtttcaccatgttggccaggatggtctcgatctcctgaccttgtgacccaccctcatcggcctcccaaagtgctggaattagaggcgtgagccaccgcgcccagccgccaatgccatcttcatcccccagatagacagtctctaggatctgttccctggggctgagcggttggagtcttcatgcgggccctctggcccatggctcactaggtctgtgtccacatccctccagagcatcttacaggcgctggatgagctgctggaggcccctggggacctggagaccctgccccgcttacagcagcactgtgtggccagtcacctgctggatggcctagaggatgtcctcagaggcctgagcaagaacctttccaatgggctgttgaacttcagttatcctgcaggcacaggtaggtccctgggtctgccccagactccagctctgcatgttttctgtctccttcctttcccagtcccaccagagccaagtgaccacacctgtatatcagtgttactctcatcgacaaactaaaataagagattaaaaagaaatacataccagtctggacaagatggcaatactcaatctctacaaaaaaaaaaaaatcttttaaattagacagatgtgatggcacaactgtggccccagctagttgggaggctgaggtgagaggatcgcttgagcccaggaggttgagtctgcagtgaactatgattgcatcactgtactccagcctggttcacagggcaagaccccatctcaaaaaagagaagacgaagaggaggaggaggaagaagaagaagaagagaaggaggaggaggtgaagaggaggaggaggataaagaggaagaaaaaggaggaagagggcaggagggggaggacagaaggaggaggacaggaggagaggaggggaggaggaagaggaggaaagaggatgggaggaggaggacaagaggaagaggacgggaggaggaggacaggatggggaggagaggaggaggagagaaggaggaggagaggaggaagaggaggaatggagaaagagaggaggaggagaaaaggaggaggagaggaggaggaggagaggagaaggaggaggaatggaggaggagaggaggcagaggaagataggaggaggagaaggagaagaggaggagaagagaaggaggagaggaggaggagacgagaaagagcggaggagaggaggagaggaggaggagcacaggaggaggaggagaggagtagaggaagaaggaagaatagaaagaaggaaaggaaaggaaaaagaaatgcataaataacatgcattttcattgtaatatgaattatcagcagtaaataattatgatgaagaatttaaaataacaaaatagaaatcacttcaaaagagaacaatttatttaatttggaatactataaattaatatcatttgatctctaagtatgatttctttcttgtttgtttttgtttgttttgttttgttttgttttgtttttttgagacagagtctcactctgtcgcccaggctggagagcagtgacgtgatctcggctcactgcaagctccgcctcctgggttgacagcattctcctgtctcagcctcccgagtagctgggactacaggtgcccgccaccacgcccagctaattttttgtatttttagtagagacggggttgcaccgtgttagccaggatggtctctcgatctcctgaccttgtgcataatccacttgcctcggcttcccaaagtgctgggattacaggcgtgagccaccacgcccggcctgcataataatttttaaagcatgaaaatgcatatagcattaattttattaagataaaattcacataacatcaagttcaccactttaaagtgtctgtttcacgtggggcgtgcctgtaatgccaggcgctcacacctgtaatcctagcactttgggaggctgaaatgggtggatcacttgaggtcaggagttcaaatccagcctggccaacatagtgaaaccccatctctactgaaaatacaaaaattagacaggcgtggtggtgcacacctgtaatcctagctactcaagaggctgaggcaggagaatcacttgagcttgggaggcagaggttgcagtgagccaagtttgcaccactgcactccagactacgtgagacagagtgccactccatctcaaacaaaaaaattaaaattaaaaataaaaataagaaaaaattttaaaagcgtgtgtttcggtagaatctagtatattcagaaagttatacaaccgtcacctctatttctaaaatgcttgcatcagcccaaaagtgaacactgcacatggtaagcaaccactcctcattcctcccttcctccaacccatggcagccgctaatctgctttctgtttccatggattttcctaatcaggatatctcacataaatggaatcatacaatatgtgaccatctagtctagctcctttcacctagcataatggttttgaggttcatgcatgccatagcatttatcaatatttcattctttttcatggctgaatactattctattgtatgaatacatcactttttttttttttatccatccatccattgatggacatttgggttgtttccaccttttggctgttgacaaaaagagtcaaactctgtaaaatatttgaagagatttattgtgagctaaatatgaatgtatgactgaccatggcccgtgacacagccctcaggagcccctgagaacatattcccaagtgaagtggtgttgttttctggggtaatacctgaggttcattgcctcatgccaagaaaattaaggacacagacacacaccaggagtgagtttatgagtggaggtttaataggcaaaagaaagagaaaggagaacaactctctctcttgtgagagaaaggaatgcccaaatgggacttcccacccatgccagagggcaccggattttatagacaggcttgaggaggcagtgtctaatttacatagggcccaaagattggttggaacaggtgtgacgtttacataaggtgcagggaagctggctgccccaccctaatcttattatgcaaatggagtcttcgccagctccttactgtatacgtggttggcaaagagatgggaagatggagccgccattttcaacatgcctagtcccaggtggccttttcctattggcacagctgccagcattcacctgtgcaggcttccagcttgcttgcctatgtctgcagctcgattttacaggctgctctttgttagaaaaaaaaaaaatgatataggggctgattttcattaaaaggaaagacttacccaggacttccttacccacactctctgcctaaataattcctttttaacttctatatcacaaggtagtcgtggtacagcttgttgtgattgttgttgttgttgtcttctgttggagacagagtcttgctctgtcaaccaggctggagtgcagtggtgcgatcctggctcactgtagactccacctcccgggttcaagcgattctcctgcctcagcctcccaagtagctgggactacaggcgcccgccaccacgcccggctaattattttgtgtgttttcagtaaagacagggtttcgccatgttaaccaggatggtcttgatgtcctgacctcgtgatccgcctgcctcggcctcccaaagtgctgggattacaggcgtgagacaccacgcctgcccccgcaacttggttttatacattttagggggacatgagacatcaatcaaatatgtttaagctatacattggttcagtccagaaatgcaggacaatttgaagtggaggtgtggggcgggtgcttccagattataggtagatttaagcttttctgattggcagttggttgaaggagttattatccatagaaaagaatgtctggaatacgataaggggctgtaaaggctgaagttgtatcatccagatgaactcttcaggtatcaggcttcagagagaatagatggtaaatgttccttattaagcttcaggtctgtgttatgttaaatgctggtcagcttttcctcaactccaaaagggaggagggcatgacacatgtcagaccccccacttcccaccatagcctgaactagtctttccagttaaatttagagtgccctggccaaaaaggaagtccattcagatggttttgaggggggcgggagaggacttcgaattttttttttttgtttagatggctggtgtgaatagtgctcttatgaatattagtggacacattttgggttgaataactgtcttcagttctttggggtatatactaaggagtggaattgctgggtgatatggtaattctatgttcagcttttggaggaaccaccaaactttttccaaagcagctgcatcattctaattccccaccagaaacatacacgggttatcctctccacaccttcagcaaaacttgttacttccgttttcttttgttctgtttttaattgcttttattgatacataaatcttacatatttatgggtacatgtgatactttgttgcttgcctagaatgtgtaacgatcaagtcagggtatctcaggtatccaccactttgagtacttatcatttgtatgtgttgggaacaattgaagtcctcttttgtaactactttgaaatacatcatacagtcttgttaattatagtcactctgctctgctgtcaaacaatagaacttgggccgggcacggtggctcacgcctgtaatcccagcactttgggaggccgaggtgggaggatcacgaggtcaggagatcgacaccatcctggctaacagggtgaaaccccatctctactaaaaatacaaaaaattagccaggcgtggtggcgggtgcctgtagtcccagctactcgggagactgaggcaggagaatggcgtgaacctgggaggcgaagcttgcagtgagccaagatggtgccactgcactccagcctgggtgacagagtgagactccgtctcaaaaaaaaaaaaaaaaaaaaatctgcttaataagcggagaggtggctggcttggtggctcacacccgtaatcctagcactttgggaggtcaaggcaggcagatcacctgacatcaggagtttgagcccagcctggccaatatggtgaaaccctggctattaaaaatacaaaaattagctgggcttagtgacacgtgcctgtaatcccagctacctgggaagctgaggcaggagattcgctggaacccaggaggcagaggctgcagtgagctgagatcgtgccactgcactccagcctgggtgacagagtgagactccgtctaaaaaaaaaaaatatatatatatatatgtataacttatactttttgtctaactgtatgtctctacctgttaactcacctctcttcatcccagtgatacaggagtgaaaaagaaattatttaggcagttagcaagggtcagagagtcctcagtaaggtttcccttttaataaaaaccagcccccaaatcatttcttttctaataacgagcagcctgaaaattcgagctgcagacatagaaaagcaagctggaagcttgcacgggtgaatgccggcagctgtgccaataggaaaaggttacctgggggcccgacatgttcaacgtgggggctccctcttcctttatctttgtcaaccacgtgtacagtacagaagcaggcaacgtggagccagccaggtagagaatccatttgcataataaaagattaggctggggcagccagtttcttcatgctgtatgtaaatggctcacctgttcagaccaatctttaagccttatgtaaatcggacaccgcctacccaagctcatctataaaacttcgtgcatttcaccacgcaaccggaagacccacttgggagcccctgtctctctgcaggagagggagttattctctttttcctttttttttttttgagacggagtcgtgctgtgtcccgcagactgcagtgcaggggcgcgatctcggctcactgcaagctccgcctcccggggtcacgccattctcctgcctcagcctccggagtagctgggactacaggcgcccaccaccatgcccagctaatttttttgtatttttagtagagacggggtttcaccgtgttatccaggttggtctcgatctcctgacctcgtgatcctcccgcctcagcctcccaaagtgctgggattacaggcgtgagccactgcgcccagccagagctattctttttttctttcgcctattaaatctccactcttaaactaacttcttatgtgtccacatcctcaatttccctggtgtgaaacagtgaaacttgggtatttacaccacttcactagtgagatgatacctcattgtggttttgatttgcccttccctgataactaatgatgtttaacatcttttcatgtgtttgctcaccatttgcatgtcatttttggagaaatgtctattcaaatcctttgcctatttttaattgtgctgtttgtcttttggttgtccagttgtaagcattctttaaacattctgatagtagatcatctgagatgaatgatttgcaaaatattttctcccgttctgtagattgtcttttcattttcttgtcttttcactttaatgaatagaagttttaaatcttgaagacatttattttatctaatttttcttttgttgcatgtgcaattggtgtcatatttaagaattcattgacagggggtggtggctcacgcctgtaatcccagcactttggaaggccaatgtgggcggatcacctgaggtcaggagtttgagaccagcctggccaacaaggcgaaaccctgtccttactaaaaataaaaataaaaataattaaccagatgtgttggcacatgactgtaatcctagctactcaggaggctgaggcaggagaattgcttgaacctgggacacggaggttgcagtgagctgagattgtgccactacactccagcctggatgacagagcaagactctgtcccaaaaataaaaataaaaaaagaattcattaccgcatccaagaccaggaagatttacccctgtttcctcctaagtgttctagttttagcttctaagcttaggtcactggcttattttgagttaatttttgtttgttgtgtgagggtagggttaaaacttccttattttcatgttactatccagtaatcccagcactatttgttgaagagattattctttcccccattgtatagtcttggcaccccttgttgagaatcaactgaccataagatggatgggtttattcctggactctcaattctattctgttgtgtccttatgccagtattactgtcagtttgtagtaagttttggaatcaggtagtgtgaattctccaaccttcttgttcttcttcaagattgtttttgggtatttggattccttacaacaccatatgaattttaagatgaacttttcttattctgcaaagtaataataggctaaaatattgatagggattgcattgagtctctagaccactttgggggagtattgttatcttaacaatagaaagtcttccaatccatgaacataggctgtctttccatttagagagggccttttatttctttcaaacacttttgtaggccagaagcagtggctcacgcccaatattgtcagaggccaccaaggtgggagaattgcttgagcccaggagttcaagcccagccttggaaatatactgaaacatgatctccacaaaaaatttaaaaattagctggctgtggtagtgagcacctgtagtgccagctacttgggaggctgaggtggaaggattggttgagcccaggagactgcagtgagctatgatcatgccactgcactctagcctgaccaacaggtgagaatttgtctcaaaaaaaaaaaaatactcaaaaaactttcctataagtcttgagtctccttcgtaaaatgtattcctaacatttttgctgaaaatttttgcatctatgtccataagaaatattggtctataattttattttcttgtgatgtcttcatctggttttgttatcagagcaatattagactcataaaattacttaaatagtgtttcctcctcttccatatttttaaaaaggtaaaagattcatgttcattctttcttaagctattggtagaactcaccagtgaagtcatctggtctgggctgttagttttgagaggttttgattactgatttaatctgttataggtctgttcagatcttctatttcttcttgagttgatttcagtggtttgtgtgtcactaggaatgtgtccatttcatctaggttacctactttgtagatatacagttgctcacactattctcttccgatattttgtaacttcagactatatatgtatatagtatatatattaagtcatagtatatcaaccactgcaggcacgattcctaacctaaccccccttatgtaattcccacaactcatagaattgtccctggaggtgcagaagcaagtagacaggagtgtcaccttgagacagaatcaggcagtgatgcagctcgactggaatcaggcacagaaatctggtgacccaggtaatggctgagatggggaggtggtcagtgaagcttcatggtgggaaaagatgcccaagaatgtgaaaaataagaaatttaaagattaaagggaagtgtgggatgtttggagtggggctgtaagaggagagtccagctgaggaggaacggaccttgttggggaccctgagatattaaccgtggccttgtcctgcaggatgtgatacacatagaactaaagattgaaggaaagtgtgggaggggtgaagccaaaggaaggtagcccatctgagtagaaagttctgtgcagggcagaggaactctggtgtggacccagagattcaacctactccaagattcaaccttggcctcgccctgcaggcccttctgtggtgggccttgtctccattccagggatgggcaagttgctggctgaggcccctctggtcctggaacctgagaagcagatgcttctgcatgagacacaccagggcttgctgcaggacggctcccccatcctgctctcagatgtgatctctgcctttctgagcaacaacgacacccaaaacctcagctccccagttaccttcaccttctcccaccgtgtgagtgctggtggagttggttggtgggtgaatagtctgagtccgggcatagccttgctgctcagctcagccctggggctcaggggtctctgttatgggtacattatctccccaggaaagtcagtccttcccaagccggctttgggtaagcatttctgagcatccgacccaccagctcacacctgtgttcttttttttattgagacagagtctcgctgtgtcagccaggctggagtgcagtggcacgatctcagctcactgcagtctccgcctcccaggttcgagcgattctcctgcctcagcctccctaatagctgggactacaggtgtgtgctaccacgcccagctaatttttgtattttttttttttttttttttttttagtagagatggggtttcaccatgttggccaggctggtctcaaactcctgacctcaagtgatccacctgcttcagcctcccaaagtgctgggattacaggcgtgagccaccatgcccagcccacacctgtgttctgttcctgcagtcagtgatcccgagacagaaggtgctctgtgtcttctgggagcatggccagaatggatgtggtcactgggccaccacaggctgcagcacaataggcaccagagacaccagcaccatctgccgttgcacccacctgagcagctttgccgtcctcatggcccactacgatgtgcaggtgagacccttaggaggggatgcactctgcatttattgccgtgtaacaaatccccagagacgtagcagccttttaaaaaaatatatgcattatctcacagctctgggtcagcaagctggcatagcaagatggcttttctgctcagggtcttacaagactgaaatcatcatgtcaccccacccccagggctgccatttcatctgagacttgaggtccttctccaagctccctggttgctggcagaattcagtttcttgtgattgtaggactgaagtctcactttcttagctgtcaggaaggcatcacattcagttcctagaggccactcatatcccttctcacatgaccctctggcaaacttctagcattcctatctggaggacaaggaaaaaaatttcctcacgcagaatccctcttaagctttgtattagtcgaggttctctagagggacagaactaatggaatagatagatagatagatagatagatagatagatagatagatagatagataatttctgcctgcttatattctagccacgctggcagctgattagatggtgcccacccagattaagagtggatctgcctttcccaacccactgactcaaatatatacatatatagagatatatagagagagatctatatatatagatagatatagatatatataaaggggagtttattaggtattaactcacatgatcacaaggtcccacgataggccgtctgcaggctgaggagcaaggagagccagtccgagtcccagaactgaagaacttggagtctgatgttcaagggcaggaagcatccagcacaggagaaagatgtaggctgggaggctaggccagtctctcttttcacatttttctgcctgcttatattctagccacgctggcagctgattagacagtgcccacccagattaagagtggatctgcctttcccagtccactgactcaaatgttaatttcctttggcaacaccctgatagacacacccaggatcaatactttgtatccttcaatccaatcaagttgacacccagtattaaccgtcacaagttttgaatctgattctccaggaagagcccagagcttataaagactcatctgattaggtctggcccacccagggtaaactctgccttttttttttttttagacagggtctctgtcacccaggctggagtgcagtggcacagtcatagctcactgcagccttgaccttctgggttcaagggatcctcctgcctcagcctcctgagtagctgggactacaggcatgcaccaccatgcccggctaatttttgtatttttactagagatggggtttcaccaagttggccaggctggtctcgaactccttacctcaagtaatccacccgcctcggcctcccaaagtgctggtattacaggcaggaaccaccacacctcgcccacctgactttcttatctcatcacttagagtagatccaagggacatcatcatcatatcctgcgggagaaagggccaaaccacccttttgttttccatgacaggaacatcttatcaatatcctcccaggcagcaagccataccgcccagcccctcccgcccagacctgtaattaccccagcctgtaagcggcagtgggttctggcacgaagctagctcccccctccacaagtctcctgctggacataaacctgcattgctgtagagctgccaactctctgtctttctttaactctcactttcccttcaaaacctaacgggcccacacctggaatctctgtgctttgggaggctgaaacgggaggatcacttgagcccaggaattcgagaccagcctgtgcaacacagaaagaccctgtctctacaaaaataaaaaaaaaattaggacgggtgtggtggctcacgactgtaatcccagcactttgggaggccgaggcgggtggatcatgaggtcaggagttcaagaccagcctggccaacatggtgtgaccctgtctctactaaaagtataaaaattagcctggcatggaggcacgtgcctgtaatcccaactacttgggaggctgagacacgagaatcgcttgaaccgaggagacagaggctgcagtgagcagagatcgcaccactgcactccagcctgggcgacagagcaagactgtcaaaaaaaaaaattagcagggcatggtgccatacatgactataatgcacacccgttgtcacctacatgactacgcctgtattcctagccacccatgaggctcgggtgacaccttcgcaaattaaattaaattggtgacatcttcacaaatttacctggctccctcattcttgaatgtgctatgccaaagtaaaaattttaaatggtggtggggtcattaccccagagcaggaaagcatcactgtgaactcttcttttccgggtcccaggaggaggatcccgtgctgactgtcatcacctacatggggctgagcgtctctctgctgtgcctcctcctggcggccctcacttttctcctgtgtaaagccatccagaacaccagcacctcactgcatctgcagctctcgctctgcctcttcctggcccacctcctcttcctcgtggcaattgatcaaaccggacacaaggtattgacagcggtgttccagagggctcccttccttgacgcaggacacgctggctcatggaatgatgcggcatttgattccttaatataatagcccggctgggcgtggtggctcatgcctgtaatcccagcactttgggagactgaggtgggcggatcacctcaggtcgggagtttgagaccagcctgaccaacatggagaaaccccgtctctactaaaaatacaaaattagccaggcatggtggcgcatacctgtaatcccagttactcaggaggctgaggcaagagaatctcttgaacccaggaggtggaggttgtggtaagccgagatcgcaccattgcatgccagcctgggcaacaagagtgaaattccgtctcaaaaaaaaaaaaaaaaaaatatatatatatatatatatgtgtgtgtgtgtgtgtgtgtatacgtatatatacacgtatatatatataggcccttgagtcctggggaatattagaagagaacaccaccaatttcccattgcctgactcaactacacagcaaatattctctataatgccatcaaatatctcagaattctccccaacaacatggtacaggagaagccatgggccacactcaaatctgatcgccgctctgggcgtcttttacttctccgccaggtgctgtgctccatcatcgccggtaccttgcactatctctacctggccaccttgacctggatgctgctggaggccctgtacctcttcctcactgcacggaacctgacggtggtcaactactcaagcatcaacagattcatgaagaagctcatgttccctgtgggctacggagtcccagctgtgacagtggccatttctgcagcctccaggcctcacctttatggaacaccttcccggttagtgcaaattctcacaatccttatcttctccggcataaccatggccattgctagaacctagatagcatttttatgacacctgcttttaattagatcagtagggattttaggctctattggttggattacactgcagctccttattcttagctattgagtttatttcaattacatttcaagctaggcattgtagaagagggttttcggaaattttctgaatgaaataaagggcaagcctttggaaagcacgaataggacaaagacactatgtcataatccctagggcatctttctgctagaagattataataattaaggagggtcaggctcagtggctcgcacctggaatccctgtgctttgggaggctgacacaggaggatcacttgagcccaggatcttgagaccagcctgggcaacacagcaagaccccttctttacaaaatttaaaaaattagcagggcctactggcacacacctgtagtcctagctactcaggaggctgaggtgggaggatcgcttgagcccaggagttcgaagttgcagtgagctatgattgcaccactgcactccagcctgagcaagagagaaagaccttgtctctaaaaaataataataatagttagggataatgcatttaaatctgtctcactgaaatagtaagaatgaaatcaaagtgctcaagacagcattttttaaaaatctaacctactttatctaaatcttttaggatactccaatttttcatttgaaaaagtagacctgatttgaataactaaaataattgatattgtcatagttaagatttatgagatgcttactttgtgccaggtacttttctagctagtttgtttgtttgtttgtttgccaattgcactggttgaaacctccaggacactgttgactagaacagcagacaattttgtcttgctcctgtacttagggagaaagcattgactctgtcctcattaatgtatgatgttagctgaggatttttcatagataccctttttcaggttaaagaaattcttttctattcctcatgtgttcggtattttgatgaagaaagagtgtttgattttgtcaaatgcttttccgaagcatataatttttaattatatggggtaaaaattatatgatcacataattagataatcatataattatataattatattagattattttatatgtaatttttatataaatatatatctatgtaatttatatattaatattatataactataaataattatataacatagctataaataattatgtaactatgaataattacataacatagctataaataattatgtaactatgaataattacataacatagctataaataattatgtaactatgaataattacataacatagctataaataattatgtaactatgaataattacataacatagctataaataattatgtaactatgaataattacataacatagctataaataattatgtaactatgaataattacataacatagctataaataattatgtaactatgaataattacataacatagctataaataattatgtaactatgaatacataacatagctataaataattatgtaactatgaataattacataacacagctataaataattatgtaactatgaataattacataacatagctataaataattatgtaactatgaataattacataacatagctataaataattatgtaactataaataattacataacatagctataaataattatataactataaataattacataacatagctataaataattatgtaactataaataattatataacatagctataaataattatataactataaataattacataacataactataaataattatataaccataaataattatattacataactataaataattatattacataactaaataattatattacataactaattatattacataactataaataattatattacataactataaataattatataactataaataattatattatataactataaataatatgatcacattaaattgtataatcatatattatcatatcattataattatattatatatgtatatatcatatataattacatataattatataagcataattatatgcttccttaattctttattttattaatacacatattaatagaataaaggattaaaattatatgatcacattataattatatggttatataattatatggttatatatgattaattatttacctttgagtcctggggaatattatatggttatatatggctaattatatggttatataattataatgtgatcttataattatatgattacataattatataactataatgtgatcatataactttaatcctttattctattaatatgtgtattacattgatggatgattagatgttaaactagccttacattcctgggataattaccacctggtcagggtatataatccttttaatatgttgcaggacttggtcggctagtatttttttgaggatttttgcattcattttcataagggattttggtctgcagttttcttttcttgtgatgtctatttctgggtttggtgtcagagtaacactggcctcgtagaagatttggaaagtgcttcctctttttccgtttttggttttgtgaactattaacactaatcctccaaaagttaaccatagagctaccatatgacccaggtatatacccaaaacaaatgaaaacgatgtccacaaaaaaacttgtacatgatgcagccataaagaataaggagatcggccgggtgcggtggctcacacctggaatcccagcactttgggaggccgaggcgagcagatcacaaggtcaagagattgagaccatcctggccaacatggtgaaaccccgtctctactaaaaatacaaaagttagctgggcgtggtcgtgggtgcctgtaatcccagctacttgggaggctgaggcacaagaatcgcttgaacctgggaggtggagtttgcagtgagccaagatggctccattgcacttcagcctggccacagagcgagactccatctcaaaaaaataaaataacaagatcatgtcctttgcagcaatatggatggagctggaggccattattctaagtgaattaatgcaggaacagaaaatcaaataccgcatgttctcacttataagtaggagctaaacattgggtacccatggacacaaacaaggaaacttcagacaccgaagcctgcttgagggtggagagtgggaggagggcgaggataaataaacgacctatagagtactgtgctgatgaccagggtgacgaaataatctgtacaccaaaccgccgcaacatgcattttacctacataacaaacctacaaaatgtacctctgaacctaaaataaacgtttagaaaaatgttgtacatgaatgctcatagaagcagtatttagaatagtcaaaaattggaaacaatgcctatcactgatgaatgaataaacaaaacgtaatatatccacaatgaaatgttattcagccataaaaaggaacgttaagagaccattcagtcattatttatttatttagaaacactctgtcacccaggctggagtgcagtggtgccatcttggctcactgcaacctctgctctctgcttcctgggttcaagcaattctcatgcctcagcctcccgagtagctgagattacaggcatgcgccaccacacgcagctcattttttgtgtgtttttagtagagaaaggatttcaccacactgggcaagctggtctcaaactcctgatatcaaatgatccacccgcctcggctcccaaagtgctgggattacaggtgtgaaccactgcacctggcctcattcagccattttaaaatgctgcaatataggtcttgctctgttgcccaggctggagtgcagtagtgcaatcacagctcactgcagtctcgacctcctggactcaagcgatcctcccacctcagtctcccaagtaactgggaatacaggtgtgagccaccacacccggctatttttttattttttgtagaaatggggtcttgctatgttgctcaggctggtctcaagctcttggtctcaagcaatcctcccacctcggcctcccaaagtcctggtgtgagccactgtgcctggcacaaccttccatttttgtttcattgtttgaatgtgtctgattgtgtgtatcttccttgagttgaaattttggggatctaagaccgtgttcattttctccaccactgtatcttcagagccaaccacagaccctatcactaaagaaacacttaatattgttgacgtaggaaggaagtgaggaagtggtgaggagggatggaatagatttacataactgttccatatctccaaaaactgatgcattttttttaatctctcttttttcagctgctggctccaaccagaaaagggatttatatggggcttccttggacctgtctgcgccatcttctctgtgagtgacatcatctgagcatcctcgctgccccgctgagggtcattagaaaaacaagataataaagatggtgacaagagcaggaatgtcccctgggttgtcatgggttgggttaggtttcctaagaccactcttgaatcttcaaactcaacactgttactcaatatttaagaaaaagacttacgtgtataattcatattagacaggctaggatgaagagataatgggcttcaggcccggtgtggtggctcacgcctgtaatcccagcactttgggaggccgaggcaggcagatcacttgaggtcaggagttcaagaccagcctggccaacatggtgaaaccctgtctctactaaaaatacaaaaattagccaggcgtggtggtgggtgcctgtactcgggaggctgagacaggagaatcgctcgaacccgggaggcagaggtggcagtgagctgagattgcaccgccacactccagcctgggcaacagagcaagaccctgtctgaaaaaaaaaaaaaaagagataacaggtttcaaaatctattcaacaaatcttctgtggttttctgaaggtgaatttagttctctttctggtgactctctggattttgaaaaacagactctcctccctcaatagtgaagtgtccaccctccggaacacaaggtaagatggagaagggggtgataaccacacagacatgtatggttcccaagagccaagccatgtgcctcagcagccactgaggtcccttctcagggtcttttgaagggacccagaaaaaacagacgcagccatccttctatggcagccatcacagattcagtggtatcctgttgagaccccatgcaacatgttttgtccatgtctaatctcagtttgtgaatcccagagcaacaggaagaatcttcattgactggtaaatggtatatatacaaaataggatacgattcgatcacaaaacagaatgaaaccatgtcatttgcagcaacatggatgaaactggaggtcactctcttaagtgaaataagccaggcacaaaaagacaaataccacaggttgtcacttacatagagaggagccgaagaagctgatcttgtggag

SEQ ID NO: 2022 - DNA, 56 bp, synthetic construct
  misc_feature 1..56: note = Synthetic
  source 1..56: mol_type = other DNA
tgctactgctgcccctgctgtgggcaggtgagtggctgtggggagaggggttgtcg

SEQ ID NO: 2023 - RNA, 100 nt, synthetic construct
  misc_feature 1..100: note = Synthetic
  source 1..100: mol_type = other RNA
acaggcccaggacacagagcgttttagagctagaaatagcaagttaaaataaggctagtccgttatcaacttgaaaaagtggcaccgagtcggtgctttt

SEQ ID NO: 2024 - RNA, 100 nt, synthetic construct
  misc_feature 1..100: note = Synthetic
  source 1..100: mol_type = other RNA
cactcacctgcccacagcaggttttagagctagaaatagcaagttaaaataaggctagtccgttatcaacttgaaaaagtggcaccgagtcggtgctttt

SEQ ID NO: 2025 - RNA, 99 nt, synthetic construct
  misc_feature 1..99: note = Synthetic
  source 1..99: mol_type = other RNA
gacaaccaggagaagatcgggttttagagctagaaatagcaagttaaaataaggctagtccgttatcaacttgaaaaagtggcaccgagtcggtgcttt

SEQ ID NO: 2026 - RNA, 100 nt, synthetic construct
  misc_feature 1..100: note = Synthetic
  source 1..100: mol_type = other RNA
caaatccatctccttggacagttttagagctagaaatagcaagttaaaataaggctagtccgttatcaacttgaaaaagtggcaccgagtcggtgctttt

SEQ ID NO: 2027-2031 - skipped (5 entries)

SEQ ID NO: 2032 - protein, 113 aa, synthetic construct
  REGION 1..113: note = Synthetic
MADYKDIVMTQSHKFMSTSVGDRVNITCKASQNVDSAVAWYQQKPGQSPKALIYSASYRYSGVPDRFTGRGSGTDFTLTISSVQAEDLAVYYCQQYYSTPWTFGGGTKLEIKR

SEQ ID NO: 2033 - protein, 127 aa, synthetic construct
  REGION 1..127: note = Synthetic
EVKLVESGGGLVQPGGSLSLSCAASGFTFTDYYMSWVRQPPGKALEWLALIRSKADGYTTEYSASVKGRFTLSRDDSQSILYLQMNALRPEDSATYYCARDAAYYSYYSPEGAMDYWGQGTSVTVSS

SEQ ID NO: 2034-3031 - skipped (998 entries)

SEQ ID NO: 3032 - protein, 123 aa, synthetic construct
  REGION 1..123: note = Synthetic
DIQLQESGPGLVKPSQSLSLTCSVTGYSITSAYYWNWIRQFPGNKLEWMGYISYDGRNNYNPSLKNRISITRDTSKNQFFLKLNSVTTEDTATYYCAKEGDYDVGNYYAMDYWGQGTSVTVSS

SEQ ID NO: 3033 - protein, 107 aa, synthetic construct
  REGION 1..107: note = Synthetic
ENVLTQSPAIMSASPGEKVTMTCRASSNVISSYVHWYQQRSGASPKLWIYSTSNLASGVPARFSGSGSGTSYSLTISSVEAEDAATYYCQQYSGYPLTFGAGTKLEL